SEQUENCE LISTING (113 sequences)

SEQ ID NO: 1 - DNA, 25 bp, synthetic construct
  source 1..25: mol_type = other DNA
gtaagagagctcgttgcgatattat

SEQ ID NO: 2 - DNA, 33 bp, synthetic construct
  source 1..33: mol_type = other DNA
tactaactggtacctcttcttttttttctgcag

SEQ ID NO: 3 - DNA, 23 bp, synthetic construct
  source 1..23: mol_type = other DNA
tggtacctcttcttttttttctg

SEQ ID NO: 4 - DNA, 20 bp, synthetic construct
  source 1..20: mol_type = other DNA
agatctcgttgcgatattat

SEQ ID NO: 5 - DNA, 27 bp, synthetic construct
  source 1..27: mol_type = other DNA
gagaacattattatagcgttgctcgag

SEQ ID NO: 6 - RNA, 135313 nt, Homo sapiens
  source 1..135313: mol_type = other RNA
agtccccagtctttgcttaggcccctacgtacacaaactgaacctagtgacccagcatggcctctaatttctcaacacttctgtacttctgtaatgattaacccatgcttctcacagatccatgccccaaatttctgtgaataggccctgactggcccagctaagatcatgtgactgcacatgaccagtccactttggcattaacaagcctactgcagactcttcccttggtgttggagtcactcctagaaaagagcaaatctttgtgagccaggcagtcaacctgctggcagcttccactcagccttggagttttttctatgtgtaactttcataaactgagccttatttatttattttttgcactatcatctcatgaaatattattgcgtaagctgaggaaacatgttattcatgatgactggagtttcaagttttaattgtacaatgatttagttttgagtttggtagaaataaaatcaaatttaaaaatcagatatttttcatcttacattatgatgtcccaaaactgcctttatgcttgtgacatagattcataatgtcttctcattccacctgtaatcactgtttgaaataaacatatgtctaatgatatatttggggacattctattttcttcagcttgttgcaagtgaattgatggtgatcttttggtattggtttcattatcaaatttatctccactccaaaattacagtaatttcaaagtaatttagtctatatatttttccatagcttttcttccaaatagaaactgtaaaaagttataaattacttctctccactactgaatttttgtttgcagaataactgatgtaagtagcagaatgcctcttcctagttcaaccctcaggaatagaagtgagaagatctttaaaacttcaccattttccttgacttgtttataattctgaatgtaaatgtgaattgatatggtctatcgcttaacaccacaactcttaatctatgtgcagggtcgtagctcaaaactactgccaggaccacatcaatttcatattcaccctgatcaatgtgtattaatggtgataactatgagaatgaaatgtacagttatcagtatcatttttgactcactaggtatatcctcagaaatatatgaaaaaactaaacacagcttttagtttgacataatttttaaacaactggagttaccttgggagaaaaatcctaccaaatatctataatattgaaagagtaaaaaagagttaaatgtccttaacatcattaatcattagggacatgcaaatcaaaaccacagtgaaataccatctcacaccctttaggatggtggtgataagaagaaaaacagagcataacaagtgttggccaggatgtggaaaagctggaaccattgtgcactgctgattggaaagtacaatggtgcagctgctaagggaaatagtatggtagttcttcaaaaaataaacagttataccatttgattcagcagtttcactcctaggtatataccccaaagaattgaaagcagaatctcaaatatttgtacacctatgttcatagcagcattactcacaatagccaaaaggtggaaacaacccgaatgaccctggatggacgaatggataaacaaaatgaggtctatactgacaataggatattaattgaccttaaaaaggaaagaaattctggccgggcacggtggctcacacctgtaataccagcactttgggaggccaaggcaggcagatcacctgaggttgggagtttgagaccagcctgaccaacatggagaaacctcatatctacttaaaatacaaaaaaaaaaattagccaagcatggtggcgcctgcctgtaatcccaggtactcagtaggctgaggcaggagaatcgcttgaacaggaagcagaggttgcaatgagctgagattgcaccatcacactccagcctgggcaacaagagtgaaactgcatctcaaaaaaaaaaaaaacaaaaaaaacaaaaaaagaaattctgacacatctgctatggtctaaattatgtgttcctctaaaattcataaattgaaatcctaacccccaaggtgatggtattaggaggtgaggctttgtggaggtgattaggtcatgagggtacaaccctcgtgaatgggactagtgccctcataaaaagaagcccaagagagaccccttttcccttccactggatgaggtcacaccaagaagttaccatctacatgtcaggaaataggccctcaccagacaccaaatctattggcaccttgatcttggacttcccagcctgcaaagctgtgagaaataagttcctgttgtttataaaccacccagtttatggtattttgttatagcagctcaaacagattaagatggcttgctacaacatagatgaactttaaagattatgcttttctatgattccacttagatggggtaccaagagtagtcaagttcattgagacagaaaatagagtggttggcaggggctagggggagggaactctggggagttagtgtttaatggatacagagtttcagttttgcaaaatgaaaaagttctgaagatggatggtgctgatggctgcacaatatgaatgtatcaacactactcaactgtacacttaaaataaggttcaaatgatacattttatttcatgtgtgtgtcaatctcaacaaacagatttgttcaggcaaggaaactggttagatgcgaataatactattagagcatcatcaattgaatattaacaaagtgctcatagtttaactttctagctcaaggaagaatggaccattttgaaactatgacagaacattacttatatagctgatgtctttgggaattggaaggaggcatattccttcaccagctgtggctccccttcagcaacctcatatactctccaagcttctctttcctgggtcacctgtttaatcactcccgggacttaatcttccacctatatgttgaccactcacaaatctatgtctccatctcacaagcttattcttgactccagacccaagtattcaactgcctgctgaatacgtgtggtcagatgtcatagaacttcagcttcagtatatcaaatgcaaacccctgttccccccaactgcctcctactccccactggccttcctctggcattccctcctcagttatgagcaccaccgtctcactagccagccagtcaagccccaaactccatctagctgacttctgcctcttcctcaccaccctcttccagtaactcatcaggcactgctgtgtctcattccttcctatccctccagtccctccccttctctccatcatggctgtcactgcatggttcaggctctctggctccccccaaaccacccccacattgctgccgaggtgaactgactactcttggcagccactggattaaaatctttcatcatcttcagcatgataaaacccatatcctttagcatgtaacaaggtcttaatgattctgccagagcttgcttgggggtagcctgcacttgtgggccactccagtcacttcacaggtgctcagtaaatctcagttgaatcagtcatcatcatcatcatcatcatcatcatcatcatcatcatcaatttttcagtctggttcctgtctccttttccagcatcctccattcatagcctcatagccttcactccagccatgtttcacttgtggttttcctgggcaagataagctattcctccctgtctttgcagagtttaaatgactcacttgttcaagtacccaccgttgccatgtgggaccgtgagcaaagtacttaatctcactaagcttcacgttcctcatctgtaaaacagcaaatatggacctcacaaaattgtagtgaggctaaaatgaaataacatatgcaaaagcagtttataaataataaacttactataaaatattattttgtaattctgcaagcttgtcttaaatgccatcacctccaaggagcctttttgccatcataagcagaaactatctctctcttcttggaagctccaccatgcacagcctatgggccctcatcacactccttgagttattcgagttcaagtcccgtgtttacaaccagaccgcaaactctatgaagtcagcatccattcctctctgtggttctccctccgccccatccaggtctcaagggtctagagtctttcaaagagaacacattctgagatttgaggaggcagagacaaaaagttccactgcgaagtgccagggaggcttctgtttggggtgtcccttgggatcacagatcccccacctggtgatgagtcaacccagcaccaccccattgcagggctggaatgacagtaatgggcccacctgctgcctctcctcatacccgcaccccagtcagacattgcaagtcagtcacggctctgtcctgctgggcctggagtgttccagtgccttttccatcacagcaccaagcagccactactagtcgatcaatttcagcacaagagataaacatcattaccctctgctaagctcagagataacccaactagctgaccataatgacttcagtcattacggagcaagataaaagactaaaagagggagggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgtttatgtcctttgctgtctgaggggcctcagctctgaccaatctggtcttcgtgtggtcattagcatgggcttcgtgagacagatacagcttttgctctggaagaactggaccctgcggaaaaggcaaaaggtaacagttactgtctgtggtttaaaaatgaggtgtggagcaaataaacaggttggaagtgtggggtggggtggtggggtagggtggtggggcagggtggggggttgtgagcagtcagtgggcttgtcgccgattagcactgaagcagtgtttagctggacggcctttctgtgggcccctctgacagtgcccttcccaggaagatgtgtttctctgtcctcagccacatgaaaatcttttgcctaccgtgcctgtcaatccattgcctgcccgcccctcccccaccccccgttttacacctgcctgtccagtctaccgctctctagggcatccacgctgagcagtgggaagaactttaagccctgaagagcaggccaaaggcaagcaagaaccccctcgaacagcttcccagcttagtgaggccttatttcattgattctctgaggcacattgttttttcacatgttagcatttctgaaattgggatgcagctcacgatcaagtcacagtttaactggacacattatttttctttcttagtggtgcagaaaagtaacagtgtgtcttacaattgactgcgtcctagattctgtgagatgcaatacgttattaaccatcacgcacatttcctgaactctttcaatgagcagacaccagcctgggttagactggagccctaaaagcacgacacagattccaccctggactggcttctgttctgcctgggaaaacccaaagtacgtttggagaccaagagcaacataaagtagcataggtggaatagtccatgagaagtgcgagcaaaaggtgccggagatcagagaacaccaagactgtacttgtaaatgacaactggctttgtgcaattttttctgggaaaggataaggagtgactatagaactgtaaagaaagaatgcactttgctacagccttgcagagttgtgcaaatgccgatgactaaaggagctgaaagaggaaggaggggataagggatgggggctgggtaggggtgagattaggaccctgggagctgcaagccactggagagatcaggaggaaagggagggagacctgctttaggcgagaagagaacagtatttgttccaaatctcggttcagaataagttcatgtaggtgatggggccaactggaacaggtgaaggcctatgaatgagtgtctcagttagggtctccttagagtttaatatgaaaaggtgttagctaagtacagagctggtacctgagagagtaaaaggaaactctaaggtatcatggaggtagcaattgcaggacacagctcccacccctagggctgagagaaccaagggaagagacaggaattattaagacttggagcatagatgagaggtctgtggagctgacattaggacttgggaggaaggcgtgcatggaggctgctgctggatctctgaacctgacctcgggtctggacccctgaggagaaagccctggcaggttggtgcatgtggggccgagggacaatagcttaacaaccagcataaaagagagcagcatgggacacgcttcaaccatgcgcatggatggctccaaaacctgtgtgtggctggcccaggacgcagggaggctgcagggggaagagacaagttaaacctgacttgtctgggaagcaccattgtcctcaggtcactttcctctgtcaagcctggtgctgaagttatctgttgtctccaggggccaagtattaagagtaatcagaaactcagtcctttcttctaggagcttcccttcttgcatgaaaatcctgataaaactggaaaaaaaaacctcatgattaaattttttcatgtattcattctttccttctatcaaaaaataatctccaggcaccgtgctaggttcattggtatacaatggcaacaagacctcccagcccctgcctatgtgaggcatctgtggactgcggaggaaaatccaatatgccattgttctctctttcccataagaaattacaattctcagttcattttattctcactgtgctctttgtgaccctcaaagggggtcacatgataacaggactgtagctgctggcctaaaatgagcccattcctgtggcgctcatgtcgctgtgacagagaataaccctgttttcagaatgctctggtgccctccctctcaatctggcctttcgctggcatgggtgggcgactcctgctcagggactctgccttctccacagtgtgctcccagggagatggagccactcgggctgagggccttggccagggcacctcccagggctgggcctggtctgggctggcgttcactggatgccatcctgatggcctggaaattgagatttctgtctggcacgcctcccgatggctccccacctgctaccacattccaggagcttccaggatgtctgggtaagacagaggcacccccaacagattcagtagctctgagagggatctgtggctccttcctaagcttgcggttcttctggaaacttctgcctctagaagatggtccctctaagaaaagtacaaccacccagcccataattcagctcccaggttttccctcaaacctccatgtctcctgtaagcagagcaagagtaaaatcagataccaaatttcctcattcctcagctcccaatccctaagggcataagatgaaaatcttcagatctctgctttcctccctctttttttcttcctctgttaacatttgtcaagtgttactaagtgtctggcactgtactaagtgcatcacctccctgaactctccgaacagttccacgagagaggcctctctgtgatccccccggtactgatgaggtcactgaggctccagagaaggattagtaactggtggggttggacctgggattcacacccatgctgcgtgacccaggacaggcaggcatggccgttacaccacactgacccccgtggatcgagatctatccaatagtctggtcactgatatcactaagatagagtggccatataatttatcatccaatcagggcagttttgcaagtgaaagggagcactattaataattgcactgggacaataaatgtaaaccaacactggacctggaaaactgggacgtgtgtttgccctataccaaggtaagctagacacagccactgccttcatggagttcagaaccaggcaggggcggctcccacgtataattactgtgcagcacaacgtggagaccgtggagtagaaggaaacacggatgggaggtgaggaggaggtctgtgagctcagaggaggcaccggggctggagagggtgagagaagacttcccaaggagttcatcctgataacgtgcattcccaatgacgagcgctctctccactgcacaagacaagtatacatctgcccgtgttggctgtggacctggcgctgtgtcagggagggtttatgaagatcactaggtgggtctcttggtgtcatcccttcatcccagcttctgggttaggatggatatctgtgggggggcctgaggactcatgaaagtggggcgctaatcatgttttggacaccacaccctggagcacctgggacagctgtggcctttgtcctgggttcagcatcaagccgaggatgtggcaagtaaagagaggctgggcaccaactccagtgtacccaggctccgggtcatgtttgtccaggctaagaattctgtcctggttctcagtgcagaaggaagaatcatggggctcattttaggccttggctgccttctgttaaattgaaaacagagcaggaaggaagaaaatttaacaggctcagttctaaaacaacaagcacaactgtgcccttgccagaaacccctcctccccatgttgattgaatggtaaagagaggaggggaggtgagagggagagagagagagaggaagagagagagaaaggaaagaaaggaaagaagaagaaagaaagaaaaggaaagaaagaaagaaagaaagaaagaaagaaagaaagaaagaaagaaagaaagaaagagaaagaaagaaaggagggagggagggaaggggaaaagaaaagaaaagaaaaagaaaaaaagaaggaaataccagtttgggaaaaaagaattttccaccagcccttctgagccttggctgggcttaattaaagttacagacatgtgtaaagggcagggtagggggagtctgagctgctgagaaaacatgtttttaattatactgtggaatttctccctggggtatgcctgtacgcagttaagcgtcaaggacagggatgccgctctggggaggggaagctgagcatgattttggaagccggcagaagaggctattgtgaaaaccagacctgtcaggctaggaaaagaatggctggtggtctttgaccagggagtgacgcgtgaaatgcagcaaccgcccccgccccccgccaaaaacaaacacactctcacagagttagaacaacagtgacctctcaacaaatatttttcaaagattaccaaccaaccattacctagagcagcggttctcaaccttggctgcacggtggaactacctgagacgtgttaaaaagaagaaccctgatgtcccatgccccaagattctgatgtagttgatctggggtatgatctgagaccccggcatgttttcagcctgcagccacatgagaagtgctgacctaatcaacaggggtgatgatttgaggggcggggactataggcaaaaaaaaacagcctaattcaaggatgagaagagggcacaggtgaggtgggaacagtcctagggccagacaaagaaggaagggagaaaggaggtgctgatccctcccctactcctgagaggaggcctttaagtcaccgtgccttgtggagaccagattcttcaaaaatacaagaatgagtgagtgagggagtgggtggatgccaggagagtgcgtgacaagccttgcaagggaggatgacaatgcactagcttggtttggaaattttacccctggaacaggcaggccaagctggctggtcccctccctgatacacagccctccctctttatatatggagcaggggacggtgtgtggctggtttcttagcaagcaccatggttccaagttggcaactggggagttctgaatccaaaaaggagggagatgaacgtaagtggagggcaggcctacaaggttgcagataagcttaattctgtctccttactcttctgcctttgcaacaaccctgtgatcttgcgacaaccctgtaaggcaataacaaatggctcatgtttattgagtgttacctcatgccatattgtgctttcgtgtttaacacaattgtctcatttcaccctcacgactgctctgggaggtaggtcctggtatcacatccatttcacagatgagaccatttggcacggaagagttgagtgggctgcccaaggtcacatagctaagatggaacaggctggataggaaccccagtaacttgacctcagagtaaccttctcttaaccctgagtgtacactgtaggaaaaatgagcagtcccatttcagagaggacaaaactgagactcagaggttaagcaagccccaaagtggttgttaacccagatcttcccactaactcccaaatcagcatcagtgtttaacgtaccagacctctcccagatagatgttgccgcatggaagacagccgatctacgtgatagaaagccaatattgcaagcagtcgtctaaaggagtcaaatgtgttggatttgaactggatgtctcatttctttggtgaagacactggaaacaacttccaggtttcatcaattgctcctatcactcaacgttgctatcttactgaacttgttccccagccttacccactgatggaatgatccagaatggaagacaagacaccaatgtacatgaccctgggggaggctgtttcttaaatctacagactgttggtgacctgagccccatgtcaccaaaggctttcctggagaagcctcctagaccagtcttgacaaaggctcactcattccgtggatatttattgggcacctattatgagttctgccccatgtggggtgctggaatcacagtagtgacaacgacagatgaggttcctgtcctcaggaagcttactgcccttgagggcttcacttacttggaggagtgatgaacctgaagtgcggtgtgtgttaagaagcggaagtccagggccaggcgcggtggctcacgcctgtaatcccagcactttgggaggctgaggcaggcggatcaccaggtcaggagatcgagaccatcctggctaacatggtgaaaccccgtctctactaaaaatacaaaaaaattagccgggcatggtggtgggcacctgcagtcccagctactcaggaggctgaggcaggagagtggcgtgaacctgggaggcagagcttgcagtgagccaagatcgtgccactgcactccagcctgggcaacagagtgagactccgtctcaaaaagaaaaaaaaaagtgcctcacggagagtctattcttttcttcccatattgtgtgtgtgtgtgcgcgcttcctccaacacatcctccctatatatattttgagtaaaacatcttgtaaaaagttacagctacataatcaccacctgtccctaaatagtttttgctttttctttcttcaatgcacgatcattttcccccatcaatttattttttagtttcttataatcttgttgccagtaggctgttttttaaaaagcagaacatggtttgttcttactagcaggaaaggagcatttattgagcctctgctatggtgtcttttattttgctgagagcctatttacatttctttgagaggaaaacaacaaagggttacatgaaagaccatgtgaatagcccctagctgatctattaaacttgctattccccggccagctgcttcagatctccttcagatcttatgtgtttccttcctaaggtccctggagtaagggttgcatagacctattctactctccaactcacatgtccctctccctcttcctctccataattccacatctccaacccccacccctatgtgcaatgccacagggtgtggactgccacagccactggatctgcttttggaatcaagagtccttaagctccaaatggaaccgaaatttaaataccaactttcaaccatatgttaacatcagcagcctcttccaatgtaaaaacccatggcagtgtgccctgctttgtttctttaagcaatagaaacttgaaggaagcatgttggtaggccagatttttgttggctttgcaatggatcacagtcatttattcactcattcattcactgattcattaaatgaccacatttgcaagggcaaggtaatggggagggccagaaaggacactggccccagaaacaggaggctggattttggttctgatgctgccactgctgatgtgacactgcacaggtcacctgcctcctctgagcctctttccttaactgcagagtgagtggctacagagaaatctttactacctgttagatcagcattacctgggagcttgttagaaatgcaagctctggtggggccatactgaacccaaatctgcattcatgtgcatagtgacagctaaaatgcactgaagcagatgatcttgatgatcctttatgaaagtctcatgctaatgcagttttctaaaatagaggcagagtggaacccagatggacacaaaatctggttgatataataaaacaaggtagagggtgtatggtggggagggggtaaaggaaggaaactgtttaggtaaagataccacaaccaaagtcctactgcacacatgggatctgaggagggctgtgtctgctctggttacgttttctataatctcttagcaccactgaactttctctctttttgttttgtttttccagattcgctttgtggtggaactcgtgtggcctttatctttatttctggtcttgatctggttaaggaatgccaacccgctctacagccatcatgaatgtaagcatagcagggtagcttgggcaagccctgaagagactttggtctgggccttttgtctagaaagatcttggggtgggagtgtggggatcagatctgcttatcatcatttcatgtctatgatgcatgtaacagatttatcaatgttacacaaattataatttttaaaaagtctttagagacagggtctcactctgttgccgaggctggagtacagtgttaggaccatggcacactgcagcttctatctcttgggctcaagtgatcctcctgcctgggcttccaaagtgctggaattataggcatgagccactgctcccagctaatttttttgttttttgtggagacagagtcactacattgcccgggctggtcttgaactcctggcctcaagtgatcctcccacctcagcgttctaaagcactgggattacaagcatgagccaccttgtccagcccaaattttcatgttttaatcctacacattctaagcaaatacttgtgtgtagttactaagggactgtgcacttatttttgtttgctttgttgttgctagtttttatttttttatacctaaactctctcgttttaaagagaacagatttgtagatgagttctcgaaaatatttcaggaatcaatatagagaatatgttatacatggtgccagagaaaaatgaggacaagagatgctatacaatcgtactgaagaaaaattttatttcttggacccctgaggtgtctgcagacctgaaaggaacctagtgagagcctcttttacactctgcccctgtgggaaagccttcacctggtttccggccctctatgtggtgaatgtggaagcctcaagcgttatgcaaatctgcccagtcctctattcttgatcttcaccttctcgttcatgagtttcaggccccagttctgaatcagcctcctgtccatcagactcttctttacctctccccgaggagcccataacctgcagccctactgcatgcttggggtaggtgctcagttcaccgtggttgaaggaatagacgagcgtctgctcaagcagcagcagcaactgcgtggagtcttcttgaactaacactcctatgcccctctcggcacaaaatgacgtgtccccccttgcttccccttcacatttccacccatgcctattacaacatccgtctgtctccccactacaccgggagcttgagagaagaggccatgtctctagcacccagcacagggactggcacacatgagatgctcctgcttcttaaatgctgagaatgaaggaggacatcagaggggcccgggccccttcccaaaaaggccaactcctaggtctgcatcctgcttggtctccatgactaatcccgtcttgtcctcattttctgttttaaaggccatttccccaacaaggcgatgccctcagcaggaatgctgccgtggctccaggggatcttctgcaatgtgaacaatccctgttttcaaagccccaccccaggagaatctcctggaattgtgtcaaactataacaactccatgtaagtgttgagatccctaccatgcaggggaggaagttgcacaccccttcacgtgctgaaatgcacacgtgcgtgcacggagcatggagcactgagtgttcttgtggctttgctgagcccctaacctcttaggagcagagcaggtttcctctctggaacattctgttaactgtcagggcacttggggagaaatctccaagctaaggccacgtgcacaaaatttcttggtccttatatccccagaatgtgacctggagtctgatggcagcccgctgcagagatgtgtccactgccttctggtcattgacctgcttgggtggagtgaatcattgtaggagaaaaactcagttccctcaccctgatcaacctggacagatctctcttcctttaaaagctttcttggacatctaagggctaggaaaaatgtcagggagcattgggaaggtaaatgaagtcaggtttacaaagtcaagtttacttcttgggagaaaaatacaatttccaaatcctctgttataattgccatcggccccctggagtggtgagatctcggaatatggctcgggtgcagtggctcttcactgtgggcctgcaggctattctgaaaagctgatgaaaaccaatgacccctcttccaagaaaaatggccacataccaaacattacactgtacatctgatttcagggaattgtagatgccaggttagtagcctcaggtctagggtcaaaattcaagtcgaatcccacaggaagagggtctgccttcggaattccctttcagagcattgggagaacatcatgggagcatattctagagacagaggcttagggtgtggacagggccatccctcacccactgtgctgaccttaagcagcaccttgtgcagcccatacctgaaggccaccagcaaaggcctgttggggagcaggctttacccgacctgtataaacaccaggctaggtgaaaactgagatacctggttactttagttttttccttgggggagctcagtatgattcttccaggagaagcctgcttttagactaaaaagaaaaaaagtttgataggtcaacctaatgattggaggtggccttccccactgtgaacaaactatggctgcatgtgccctacaatggcagagttgagtagttgtgatagagactgtatgatctgtaagcctgtaatttttatgtttgctgacccctggattaccagatgatagaagaggaaacatctgtcttcctagcaaagtcaaggaagtggcatttagcaggactcatattgctgcaagcactgccttgcagttttagtttacaactgcactttcagcttaagaaacacctgcccatccagagagatcgtgtggggtcacatggtgggatcagggaggcctgaagacagctcagtggaggctgcatggagctttggtgggaacggccctggcagtgtctatagatgttattgcggaaaactgaggggtgggagttggagaagggggctccagactctagctgtacttggcatttgaacccggaaagttgggtttcatgttttgcactcacattatgagtgaaatattggcttattcaaggttcttttgcttgcaaggcacggaaacccattcaagcaatcttaaaccccagaaggaaatctatgatttggatactagacattctcacagagccaagggcagcaaggcggggctcaggagaggcaggccaagacctggagagctgtcaggagctgcttcctcaactctcttccatctgggcctgccagccctggcctctgtatctactccattcacctctctccatggaccagtctcccctgctcctcaatgcctgggctgccattgttcatgcaattcacaatacctcggcctgggcaatcagaagctcatctctgaacaccatccaaattcctgggaacaaatcgggttgacccagctttattctccctgtcccatcagccttggcagaggcgtgcatgtgcatgcgtgccaatgtgtgtgtgcagggaggtccttgtggatgaagcatggctgtcagagcctacctgcgtgaatgggtggaagggcaggtctcagagaattgggtaaaaactggataaaccctccagtgatatccaccaatgtcaccctgtttaaggcttctctgggcaagagacacacagagcatgggaccgagaggcgagcagaccctgccaaaactgggagactgaatagatcgctcaccatccttgtcagttagcctatatgtacaaggaagtaaaattatctctttctcctgccttggcagtattgtaaggatactcaatgtagtagctaggccagacacatagtatctttaaatatagcatgagatggccaagcacggtggctcatgcctgtaatcccagcactttgggaggctgaggcgggtggatcacgaggtcaggagatcgagaccatcctggctaacacgatgaagccccgtctctactaaaaatataaaaaattagctgggtgtggtggcgggcgcctgtagtcccagctactcgggaggctgaggcaggagaatagcgtgaacccggggggcagagcttgcagtgagccgagatcacgccactgcactccagcctgggtgacagagcgagataaaaaaaaaaaatagcatgagatattattactgttataaaaataacagctatttccttattaatgaggctttgtccttacagcttggcaagggtatatcgagattttcaagaactcctcatgaatgcaccagagagccagcaccttggccgtatttggacagagctacacatcttgtcccaattcatggacaccctccggactcacccggagagaattgcaggtaagcatgactgcagtgctctcaagcatcatttccctcacctatggagagactgaagatataggaaagaacagggagagttggtgaaaaatatactagcggaggcaggaagggatggggtctggaggcggcttgaacatcaccttggtgaagatgcctcttcctccacagaagcctggaaggtaggaagttgggaaggaaggcaggaaaggtctcatccacgttaagtctagagacagaaagaatgctaagagagatggcactatgggaagtatgaggctaggtcaagggctagaagcaggggagacgagtttacagagtttcgtaaagatatagagcaactctcacagagttctagagcgagagctaaccaggaacatgaagcagcaaggccaactatcattaaggagccagggaggtcagagatcatgtattatcatgacataaatatgcataattgtactatttctcccagtaatatttagcacccaggccccgaggcagagcaagtggagagtgggtgatgcagggctgggggtgtgtatggaggcaccacagaaggtcaacaggcagcgggctgaaggcagggactggactacatgcatcaagtccaggctgcacgaggaaggatgagaaggcagatgagcacggaaatggactgggggaaatgaagaggcaagggaatagaagtctcagtgggtgccatgaccctgtttaagtgattgagaaaatgaacaagatgaaaaggttaatggctgtggtcagaaagtgaaatatgtgaattcaggatttcgaaggtagggtgggtgatgactggcccccagatgcggccatggtgaagtggggcaaaggtgcaggtgcatggtgaggggaaggaggaaatgggaggtgatgatgttggccccacacggacaccacggttgtgcaggaagatggcaggagctgggcaccagggtgggagccacctggagtcaggaagagtgaagagaaaggatgaagaggctccctctcctgtgtctctcctccccaggagaagaacaagaaacaatccgaaagtaataacaccaatgtgcctttacaaagtgtgagtgggtgttgtgtgctgtcacgtgtgtagtaggctcctctgtggatggctagagggactggacatggccactggatcccacttgcaagagcagaggaaaagagtggtcgtgaggaagtaaagccccccaaaatccaggggttgctgcagctttgggtgtggagcgtgccctctgaggaaaggctgctctgggggagattgcccaggaaacggggctcagaggccacgaaagcagctgttaggggcttctgggagatgtgtgctcctaggattagggagttgactctaaggatgaccttagaggttaacagggatgagaaaggggtcaccaaggggtctaccaggggaatgggagaggctgtattgatagaacagcttctgctgcaggttccaaacaagaaatgtgggagaatggttgaaatcagccccgggggcaccttcccgtgcatgcgtgcagctccttcaacattcagtcgaccttcagtgcctcctgtgagccaggcactgggctagtctctgggggtggagagatgagtcaggcaaatgccagccctcagagggctcacagggcagaaggtgagagatgagtgagcagaaaatgaccacagcgcgtgtggggcccagtggagggaaggaggggattcaggagcacaggagagtcaacaggggaaacttctccgaggagaatctgatcctcctcccatctggccaccttctgaagccctctctccccatccaagtgagaaaggacaggcgtatgaccagattggtgtatgaagatgctgaattacgttctcattgtttcaaactagtaaaccatagattttatgtagtaacttctacaaactgcattacaaacactccattctttgttgccctgggtagaagtttattttagtgagcccaagtttgaggaaccttatatggtatgagtacaattaccattttaatagtaagaaatcccccttcccctgtgtaccaaccagaaggtgtttttttcctaatttaaacaaacagatgcagacgtgggctgtccagctcctggcgggatgacatacctcatgcatccagtgggtttgatgatgaggcagacatttcacttaagtgcctgatcatcagattgagtcctgctgggaggaagtgtgaaggaagtaatttcaaaccacagtttctctgtggcttttacaatgtggatatgagaaccaaaatcactacttcttaaccccagagcaggactgattttgaattggtatgcaggcggttccttctgcaggcttcgggctgtgagaagtccctaacagagcaaatctggggacaagggctcaggaaaggttggccacggccccctaggaatgggggctctgcaagatccctggccttagaggctgtgagagggaacaggggtccatccccaagtaagggacacggtctttgaggaaatcccaggccagggcctgaagggcactgtcaggaacacaggctgtttcagtctgttgagattcaccggggcgctgctcactgtgagcacggactcctcaggccaatgtggcagaagagcccacctttgaaagcgagcgggtgggggtggcggggctggtgctggtgcgtgcttctgcacagccacctgggaaggtatgccgctggttgacccaggcagaggttttctttcatggcaaacctgcagtactgcattctcagcagggaggattaatggtaaaagaccaggcatggagcccccttccctctccctcgaagcaagctctgtggtctctcaatcatctttaaaacaccttcttcccgggagcctcctacattctcctggcttccctcccacccccaccctcagctcctggggcctcagcagccccacccccaagcctctaatcttcccagggaagggaacaagaagaaccacattttaaacgaaatttatttttctttcctcaggctcccagttcacatttctccctcaggagtctagggaagcttctgtctggtatcggcctcctcttcacctgggcccccgccctcctcaggtgtaccagaagccagcacactcccccttcccccccagagccacagcagccctgtctcctgggtggtcttgtgtgccaagcctgggcaacatcactcccagcttttcttgttttgccccttctccccagcaagatatttgtatgtaaggtcaggtgagtgagttaaagaataacgaagagataaacagtcaaatggagtcctgactgtcaggtcaagacaacagttatttactgaatgcctcatgtcattcaacagacatttattgagactctgattggatgtcagtctttaatgctgggtgtcagagagaggtgacttcaagggcttgcatctgtgcacccagcattgctaggtacaatgaggagtataataaaagcaggagccatagcccccaactctcaagagatctcccatgtgtgtatgtctgcatatgcgtgcgtgtgcatgtgtgcgcatgtgtgcatgtgtgtgtgcatgtgtgtgcatgcgtgtgtgtgtgcgtgtgttggggatggtgttggtggagtgagagtgtacaaggctgtgtatgaaggggtaattgggaaaagaacaatggagctggcacccagggacaggaggaaaagcaggagggctgggtttggaagacagccggatttatgtttttgaagagggaagactagaatataagggagcagcccttctcagagccctcctcctcccttcgggccctgtgtccagctttccccaaagtccttggatctttcctatgcaaaggggagtgacagtgggcaccactctcagggaacccattactgtgagagaagccactgtgccactgtgtggtcgaacttcaagaccggcttcccctgccccagctgcatggacaggcctgtggggttggcgcaagacccttccagaggaaactagctgcaacataaatccggatatggtgctgttcagggaaaggcacaacctggggatgagaagggtggctgtccagcacacaggggcaggcctcttggccactgggggaggggagaatttggagaggaagaggatgggatgccgtggaattgggaccaggaaagaatggggacatgtgatggttaaagctagttagagaagaactgggagataaacagtcacccatgcccctgaagcactcggggtgaagagattggcattttcacgcaccccagtgctttccctttgtgttgaagtcccttcgtagacatccaggcccataaggctcttctctggccagagcctcatgaactatagcactagcagggttgaggccaagcattggccctggaagccagccgaggaggagggtgcttgtgtgaatctcccaggaggggtaagaattatattaattcgatcataataagcatttattgagtgctgttttgaggcctgggagctaagcacttcacattccttaccccgcatcaacaatcctatgaggtagatgtggaaaatgcagacacggggacaggctcaatcacttgccccaaggtcaccttaactgttaggtgttctttatgcctccttataaagaaaccctgcttcccacaggtgttgagaggagctggagggagcttgactagggctcatcaggcaagccccggcatgtgcctggctctcctctttctacctggagcttttcctgcccttaatggccccaactcatttctcttagtccatgtcagtgccctgagcatctcagcccaagctgagatgatagaaacacccagaggggtcctctaccctgtgacagctgcggtgtgggaagagcacgtgtctcctccaatcctagaccagagtttctcagcctcagcatcactgacacttggggctagataatcctttgtgtgggggagggaggagtgtcttgggccttgcaggatgtttagcagcatctctggcctctacccaccagcacctccccagttgtgacacccagaaatgtctttagatcttgccaaatatttccaggaggatgaaattcccctgtttcagttccccagccccacctcaatgagaagcactgtcctagaccaaccccacaaagcatctgacacccccatccagccctggctaactttttccaccttcttactaaattgggcccagctgcttcagcagtcaatgtgttgggggcagcccactggcaagagcctcacctctaggggctcccagagaccccaagaacagaaccttcctctgagagttgagttacaagtgtttccaatcgactctggctgttttcctttttttgacccatttccccttcaacaccctgttctttctcttattcatatgtaggaagaggaatacgaataagggatatcttgaaagatgaagaaacactgacactatttctcattaaaaacatcggcctgtctgactcagtggtctaccttctgatcaactctcaagtccgtccagagcaggtagggggatgtcactggccagtggtccctggaggggagggaagcacccagcctgagaaaggcaagaaatatattggcttttttcttctttcttccttgtgttcacattcagaatccatcacttaatgccttgtatttagaaaaaaaccgggggatcacttgagatcgtgatcattttcaacataggattcgaagctgtacacatcctggtgaccttaaaacatctcaggtttttataactggaaggaaccttagagatcatggggcacaaccttctctttatagatgaggaaacagaaatctattcatttattactcaaatatttagggacagttgtaggtactagaacacagtgtgaaccagacaggcaaaaccccaggccagggagcttccattccagtggggccacaggcgatgctcaggtaagcagagactccgctgtgtgacttctggctgtgatgggtgctgcaaggaaaatccggtagagtcgagggttagagagggacggaggggcaggtttaagggggatgctcaggaaggccttcctgaggaggtggtatttgagcagagttgtctgtcagccacacagtaagtgagaggggagttccgggcttggaagctgccagcacagtgctggcaagtgctggggtggcgtcccgaggctacagaacctgagatgctgcagaagagcccacttctgctttcctggaccacttccttctcagcaccaggcaaactccttcttctatcccctggcacatttctgacctgtgtatacgcccccaatttatctaacccctttaaataatctcctctatttatgcagagcattcttaccactaactcacgacttgcacatcccttagctcccttactcctcacaacaatcctgagatgggtcagagaaggaggcttgcgcgtctggtgatggggtgatttgtgcacagttacagggctagaaattgtcagagccagatggaatccaggtcctctcaatcctaatccagtgtttcttacttcagtcctgtggctctcaaagcccagagaccagcagcatcagcgatgcctgggagcttgttaggaatgcaaattatcagggcccactccaggtgaactgggtccaaagccctgggataaggcctagcaatctgtgcttcacaagccctccaggtgattccgcaggctcaggtgtgagagctgcagctgtcctctgggccttctgggctccccgcccagcttcttcagtgtgatgaacacagcgagaatgctagatctgcagcagctgatatcccagacaccctcccgactccctcctggctgggtctgatcctcctccagactccaggagagaacgagacataaacagaacttcagagcctgtgttaaccctgagatcaaggtctgcacagggtgctgtctgagtccagaggagtgagggaccccaccccacctggtcagcaccagctcctggaagcaggttctcacactggttccctgcacaatgaaggagctcatacctgcttttctggcttctcagaccctgaggttttcaccgaaactagacaaggggaacctagggtcagcctggaggcagggtgagcttggcgcctgcagtgcccaggccctgggtggtgcggctccggccaggccctgtttagcttcctctcccacccccacagagggggtgctgtcggcaccgattgctcattttcccctttgctttctcttcagctcgtaaaactcaagtcctgacaatgccttgatgacttccagttggtaataaaagggagatgaagataaggacaggaatttcggggaaatttctttccagttccttactaatgtgacatttagatctctagtactgtgcttctggcatcagtgccaaggcctttcatgttggagaatggaggccggggtcaccaggttgtgcctttatttcatgttgctggctctgatgagctgatgctctgctgattagcaaacgctgagccatctgcgcttcgcagaggcacgttccagccaacccggccctccctgcccacttcccaggatgctttgccttgtgggctcacctgtcttctagctcctgatctgtatctccacctccatccagttccggggctccttatcagcactgttcccagaactgtccatcacgatggcaacgttctctctgggcgctgtccaacatgggagctcgcctctgtgttgtcactcatgctcattgaacatggatttgtgtcctttaccatcaggactggatacccctcctggtcctttctgcctggggtcttagcacagctcagaaggaacctcaccattccctctctccatctagggaattagaagatgacaggggcacagttctctggctcacccccagcccagtaaactcctggacatgcttcaaggcccagctcagatgttgcctcctcagtgaaataatttataaacccacccttctttgtcctgccttctccctcttccctactcactggagagttaacaggtgatggttaagctctgggttcaaatctcacaaggccacacacttagctatgtgacttcaggcaagttaattaaccactctgtgcctctcgtttcctcatttgtaaaatggaaatagtaaaagtgcctaccagcatggcagttgaagttaaaagaaataatatatgtgaacacttggaagggcgcctgacacatagtaaactctcagtaaatactagctgcttttagtggctattcttaacacaccctcttcagtgctctggtttcactatgttttatgggtccctgagatcgaaagtgtccacaccgactcatggtcagctgtaacctgtgcctcgtgtggggaccaggctgccatgtgtagtctggacagtgtaggaggtggcagagctcaggcctgttctgccctccagcccagagagccacgtcgttagatgtcatgggagactgtggtgccccgggaatctcacgaatttgcccacggtactcagtgtctgtccaatgctatgggagtccaggactctaggagccagttaaggtgctgggtggccacaggtccctggccaaggtccaggcctctcccctgccacctgatcctcgagaggccatcacgagggttgtacttcaagaaccactatccttgagctacctaggagctgcagaatgtgcactctgcagggcttagggcctgcagacaagatagatgcagggtgtctagttaaattcgaacttcagataaacaacaaataattttttcaaataattgtgttctattcggtccctatttgggacatatttgtactaaaaagtattcatttatctgaaattcagattcgactgggcatctggtgcttttgtttgctaaatccaagagcaaatttgttctagctacttctcaaccccaccttcagagaggaagccttgatggtactgtaacatcatgctgtaagaaggggatcccttgaattgtaaatggcactctgataagatgaggtatggggattgtattggtttcctgttgctgctgtcataaattaccacaaacttagtggcttcaaacaacacagatgcattatcttacagttctggaggtcacaagtctgaaagttagggcatcagcaggactgcattccttactgcggagttctagagaaaaatccattttcctgcctccttcagcctccagagacacgccacattctttggctagtggtctgcttccatctccaaggccagtgggggcttatcaagtctttctcacatcacatgactctgtttcttctgcctccctcttctacatttaagggacccttgtgattacacaggggcccacctagaaaagccaaaataatctccttattttaaaatcagctaatcagtggctttaatcccatctgcgatcttaattcctgtcgccatgtaacacaaggtattcccaggttctgtgggttaggacgtgggtgtctttcctaccacagggcagtttctagtgttgcctcttctccctgcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtggtaagggaggggtttggctgctcgccaattgcaaggtgattcctggggtagcagagcctcacgaattgaccttggggagggcgtgagcctggtgttctggacaatccttgcaaaagctccaggctcccagggctcaaaaaatcacaactgatagtatttctagaacagtggcccagggacccagaagtcactatgaggttcaccattaggtatgtggctgtggcatgtttgtgtccactctaaatgtggggataatcccctttacctcctctaacagagtggtaaaggaaggaggaggcctggtttgactccctgacctgctatttcctagccaggtgatcatggtaagatattgaaccttttctggtcccagtactcatctataaaacaaatataatactttacagagtggtaggaattatacaagaaaagtatacgcaaaacatttcataaattttaataaatgatggccccatgcttcttcctctggaaatggtctcaacctcaatggttggtgtttctagagagaaaaaacgacagagaaagtttcatagtctcaaaaatttggaaagccctgatctagctcaaccctttgttctagaactgcatcccagacagactgcttgggacctgaaaatatctcctcctttgctagaaggataagatgagaaggaattagataaaggaggtgtagagcagaggttttcacactgcaaagtgcataaaaaccatcagagggccgggcgcagtggctcacgcctgtaatcccagcactttgggaggccgaggcgggcggatcatgaggtcaggagatagagaccatcctggctaacacggtgaaaccccgtctctactaaaaaacacacacacacaaaaattagccaggtgtggtggcgggcgcctgtaatcccagctactgaggaggctgaggccggagaatggcgtgaacccgggaggcggagcttgcagtgagccgagattgcgccactgcactccagcctgggtgacagagcaagactccgtctcaataaaaacaaacaaacaaacaaaccaaaaaaacccatcagagaagttggtaaaagatgcaagtgctaaatccccacccccaatcactgtgattcagaagaaccaggccaggcccagaatctatcctgttaccttaggcgattctgatgaagaccattgtaggccacactttcagaaacactcaaaattagaatccttcagagaaggtggcatatataatatttctagcatggaattatgttttttttcttttgcctacattttaatttctagaactgtgttgtagggaatgtcagtcactaagaacttgattgaggaactgtgttttgtctgtttcatgactgctctctcaagtcccaggaaactcactttcagcttgtcttaaaaagcaagctgaaggcttttaaaaatgaagcaacatgaaataagacaccgcagtttctggcacggtccacgcttaatccccttcaatgtgtgactttccgtggaaagttactctacgattttcccagctcgtcagggtggggccccagagtgagtatgaagggtcagagcctagggatgccaccatcagtgagagcccaggaccccagaaaaggtctcttggctcaccacactgtaggaaaaataaaaagcaatgtagtccaaatgtctctatccaaagtttcaaaaagaacttgattttagacacgctccttgacttgttttcagaatcagacagaagagtgaggcaacaaaggtcccttattccaggcagctgaataccagcacagccaggagtccagtgctggtgtttgcagagccaccagaggctccctctcaggtgtccagggcccgcatgctttgtagaatgggcagaatgagcaatgtctgtgcacctgggctttgcaggcagggcctgggtacccaggttcgtgcaatcctctcgtcaccatgaagggagcagcatcattcttcccttcttgaagcaccttggccaccagtataggtaaatttacctcccaggacatgaccattgattctgggatgtcaatgccagagatagtagggtaaatcggcacctgggtaaaactttccattggagactagaaccaaaactcaggacactggcttccaaatgtttctttatcagacaagaaagaccaagtctttccttacgtcttcacatgctgccttggcaaatgctagcattcacaaaccctgggctaccttgacctgtcacccttgcagacctcagacgggtcctgggggcttgctttctcggtttctgtatgcaggcactcaaacctgcatcaggcacctgtgaagggccgggcactgtgctgaggccaaggctccaaatgtgaaccttccaccctcactgaactcacagccagaccagagacaagcaaacaggacatttcacagcagtgcagcctagaaagggccaacaccagcagcatttgtccccccgagcggtagcttttagaagcttccccagtgattcaatgtgtcctacaaatgcctggcccccactcccagagattctgagtcagctggcctagggtgcagccttgacttcactgtgttaaaaagcttcccagataagtccaatgtccggccaagattgagaatcactgacctagagtttaatttaccacctcagtctctatagaccacgcataataatagtaccccacacacctctgagggtccaaagaactttcatttgatcacccatgagaccaccgtggtgtggagatgctttctctctcctgttctcttaacaaagctggtgagcgacagagcctgcagtggaccgggagatggcccagaggagaaagctctgccgtagtcggcctcagttaaccacggagcaccacccctacctgctctcctctcactcctgcttccgtctcggtggagaaagatccaaccgaagcaggacacatctagtcttctggtgcctttaaaatgtacttttccatttgacaaatggattacactaaaaacaaaaatttacaaaaaaaaaaaaaaaacctgaaagaaattgcaggcattaaaatgggactttgcctttattgctcctgggcccatcctatttgggtttttagaaaaacaagcctgaggcaggcccagaaaggctcagggcagaccctccgatcctctgaaaggagcatcaggcaggcaggggttgctccggggccagggaaggggccccgctgggacgcggctgttattgcagctggttggcgcgcagccatgcttagctgcagtgcgggaatgctgggccttctgttctgggctgtttctcatacgcacgtaggccagtgtataaataaggttttattaaatgccaaatgagttctcattaacaaagaaagagggaaaatctcagtaaaccaccgtgacggcatctacccactttgagtcaggagctgggggtgtgagtgcaacctccgagacaagggaacctgtggagcccagagaatcggaggggggcgctggggttagcaccgactgagaccagctgtgttttctctcggttccttggagatcagaagtgagtgttgtcatcttcaaacaatccaaaggcagtacccatggccttactacatccctcccacaccatcccacccatccccgcgcgtacactcacacgctcatttgcacactatcgcacacgctcacttgcgtgcgcacacacagattggtgacctaggtggactgggagagaaataagagccaaatgactggattttctccaaggaaatttattaatagcccctcttggtttcacctgaaggagcttgtcttcacctgcggcctttgcaggcttaacgcccccagcttgaaacccagaagctcagacttgggcccaaggtattattagtgccaacactacctgaaatgtttcgcacctcataaaaatggtgtgtcagtttcgggtgagaggttgggacgcttcccatctgatttggcccaaggcatgcatgcccctccttctccttcccctcctcctccccctcttccccctaccatccttcctgttttctctccaactctggtgcacagctttgaaatcttgctgagaagcaaatctgtcccttctgctttgaatgtttatttgtggaagttcggcaggggaaccgaggcgggtgccaagacctgccatgctgctgggaagtctgagtctccctcccttccccctcctaaatgcttgttgatagagaaaagtcagcctcctcggcatttgggctcacggttttcctttgaaaatgcttccagtgtggcatgattcagctttcttttctgtcccccaaccactgctctgttgtcatttttacttttctgattgcattttatccgtgtctctttgactacggggtggctggacgttgagttccaggaagaaaagggcccaatcttggggttctgactacatgcgcccatcaatgtcctgtttcattcttggctctggctccctgaattcctgagtcactggggagaagcgtgggtggaccgccccctacccagtgagagttgccacagttgctgctctcctgggtcattggttgcagattgttaaacttcacctatgcatttcaactttcgggtggatattgctacgtcaagtgtctgggaaagcccccacagctacaggattttacagtgaggtcccactaatgacttgatgtcatgacttcctcattctttccaatttctcccacttctccataagggttttgggaaggggagaagagaaaggagtgattcctgagtgccagtaccagggaacagcagggctgttgggaggaaacaaaactaaatcaggaaggtttttgttgttgtttttggggggttttatgaaaatattcaagccacagcaaatatatttgatttatagcattagtattttttctgcctgcatctacaaaaatctttacctattaccatcaaaatatcctctgggtgaatggatttcaacaaagaagaaataaaaatgaaatagaagagaggccccttcgtgcacattgagcctactggctggattgtcacttgcctgccttgatgtcttttcagctccaggcaggcagtaggccagggcttattttcatgacagatcagatgttcttttatggatttacaaagaaagaaatactgagaagtcaaaactgaagtcacttaagacaagagcaggcccctgggaaggctgccattgaggataatgagtcctggggtcctggcctttgttcagtaaatacgcactaggcgcctacaatgtgtgcaccaatgtgtgaggcgtcaggttctctccagggtcagttggttttaagaaaggttttggcttctgatatgttttatctctacagaacagtagctcttaacctttcttatgggttaggattaccttcgagaatctgactacagctctagacctgttccctaaagaaaactaagttcacagggacacacaggatggggctcatggagcagctgaagccagaccccaggttaatagcctttacattaaaatgtttttctacctaccactaatatgcattctttagtaagcggtctcaatatacaccgattcttccttaactctgtttatgaagtattcagcatcctccctgcccccttcagcatcctccctgcccctgagcacaggatccaatggcgtgaggaccacaggcctgggcagctgctggggcatacaggcatctcttagtggctgagagactgggccctggctctatgttggctcctaacttgctgccatttaaaggaaatcttagcctcccatccgtaaaatcgagaaaataagacttgtcctacacagctcatgaaatagtaatgaaattcacattagagaagagatggaaaaacactttgaacaaaaagcattttgctcttataaaagcacagcctcttttgagaggccctttgctccccatttctccttcttcagacccccccagactaggagaaggtctgtctcatggagtgaccttttggctgcctctagattccaagctcagttttgctttcattaaccacagatactgggacggacagaaaaagacctagtttctgttgagccaaagagtctcataacttgtctgttcacatacccaagagcccaccctctagttgagacactcagttccctctcattctgggagactgcatgtctctgtgacctcctggtagagaccgtttgacatgtcccccaaccccccagtgattgagtctgaattctccactgatgacgcatttcctagcactcagggtgtcccctcctggttgccccctcaccactgaagcccgcttcctcccttttcatttgatgcttaacaactgtcagtttgcaagaaacatgcttcaaatccacattctcccagttgcctagcaacaacttccctcccggataaatgtgggtttcctgtagctcagcccaggactgaacacagcagcacacacttctgtccactgcttcaactgcttttcacctctggtctgcatgccttcaagactgcagctcatccctcccttcagaaccttccatagcctgcagaggccatgtctgccccaaaaagacacattgaacctgaggctacttatttacccttgtgttaggtatatcctcaacttagaaattaatactgtttccagattgtcttctttgaatcacagaaagtaaaacaacaaaacattcaatgcttaagacatttcatgtgcggttgggtgacatctgtttgatgaacacatttgatccaaagcatcagaaatactatgccaacaagactttttaggaggtgataaacatgtctgttctaccttaagaaaaaaatattacacagtcccaagggagagacatggttttgatcccagacaacccaagcagagacctcttagggccggaatcatcttggctgctgcctaggaccttatatcaatttcttaagcacaggatcaaggcctaaaggccccttagactgacctcagttagtagaggcagatcccttcacagccttatcttccttagaggtctagtctgaccttgaacttcggctggcagtgctgtcagttgtgatgtgtgacatggaagagttatttgttacttggaaaattaagagaacttatttggcataggaaattgtgtgtgtgtgtgtgtgtgtgtgtgtgtgtgtgtgtgtgtgtgtgtgagatgatgtttgccattttgatctgtgacttttttttccagaaatagtttctcagttccattccaactaaacttacagtctcttccggttctttgacagaaacaattcatgtgaatttgaacagataatagggaagggggaaccaaaagaagaggagagccctgggaaagttattttataatttatggcaacctcagtcaggcaactgtgaacaggtacatatggagggctccctcgggactaggcagtattcagagatgtaaggtgtgaggaccggaccctcatcatttaccattcccactaaaaagagctgggaaggaaattgtagctgtagcaccaggcacgtaactggagcttagtaactatttggtgaaggaatattattaaattattaacaagatggaaaaaagggtattaaccacacaaaaatacatctcaagctattgtttctctgttccctttcccccaaattcctagtcttgctcttatctggctgtctctctagtcactctttcttgctgactctcttcacgttcctttctccacctggaattcctgggccctccccttttactgacagacactgtcctcactctcacagtcatcagtttgtctctttacaaacctcagctcaagtgtcacttccccgtccccaggtgaaactgactgctccctccctgtaagtcaccatgatgactgctatatatagccctcatggaacctaaaacctcaacagacacagtctctttcctactctgttatagtttatttactcattaattaccacaacacgtattattgagcacctactgtgtaccatgcccagaagataaaagacaaacaaaataaaacctattcctatgcttaatgagtttacagtctagtggagagatagatacattaaaaaataacagcaaaccaaaataaaagtggtaaataaatgcactgagaaagacaggaatagctaggaggggcacctaatccctagggaaggaaagctggaagagcatggtgatgggggaagaaggctttctggagaaggtgaggtagtttgaaatgagttgactctggccagtaggggtagagtgagaatggggtgagacagggtgggttggtcattttgatccattagtcctcaaagtgataggactagtggctaaggactgcaggctttacagaagcctacaaaactatttgagatttgaagtttttttttttttttaattggctccaaaagaaaatgaaaaaactttagaattataatgaatgaatattaaatgaatatttaaggaaggtaattttattcaacttcattgttaaatttagttaaaacaagcccttgagtttcattcaacactgttttatcataccgttgatgagagaaaacaaaactgattcctggccagggccactgtcagcgtggggtttgcacatctttcccatgtctgcttgggttagctccaggtactcctgtttcccccacatccccaagatgtgcccattagtggaaacggtgtgtctgcatgattccaacgtgagtgagtgtgggtgtgggagtgagtgcccctgccatgggagggcatcctgtccaggttagattcctaccttgtgccctgagctgctgggatggaatccagccacccatgactctgaactgaaataattgggtgaataattatcttactttttaattaatctttgaaaatgtatgtatagttcacatgtatttcaatatttaatattagaagtattttagtctttattttgaagtttggtgatttattgtaaccagaaacaagctatagaaacttaattttgggccaagtgcagtggctcacacctataatcccagcattttgggaggccgaggcagacgcatcacttgaggtccggagttcaagatcagcctggccaacatggtaaaaccctgtctctactaaaaaatacaaaaattagccagatgtggtgggcacctgtagtcccagctacttgggtggctgaagcaggagaatcacttgaacccgggaggcggagcagtgagcagagatcgtgccactgcactcccacctaggcgacagtgtgacactccatctcaaaaaaaaaaaaaaatagaaaagaaagaaacttaattctggtttatatcaattagcctgtggtaaaattggtttcattatagccatttcacttagttgaagtttccaataacctgtggatgaattaagtgaggatttactatattcataaaatcttaaattccaaagcctgtttgcagttcaggtttttccactttacaaacacttctaagtattcacaatgattgcttaaaattcataccagataaatcattaaataagttgttcaaagtcaaataatttcataagtaaaaattaggagcttttagaaaactatacctacatagacctagacctatagatagacagagatctgaatagatatggacacagatgctttccaaagtgttcatgtgatgtgtggtggagtttcaagaccagagtgtgcctggggcctgcagaagtaaaggagaggggatggagagaagattgtccacatggccatgggcaatctcccacccacactcaagtgaggaagacaggaaacaaattcagaaagaagagaaaataatcaaaactgatgggagcttgtgactgatttacttatgcgcagcctccctggagacatgagtgtggctgttccttaggttgtgcctctgggctcctaccccctcttagatgccttcctattatctaggacctggttgctttttgtctgcatagcttctttggattccagtctttgatgccagcttcctcctaaagtagcctttcagatgtcccttggttaccctctgctatctaagggctcatcctaccccacactcattcccagcaccaatttctggatctccaggctggagatttagacaatgggatgggaagaacccatgatgggtcccagacagaaagtggtgccagccacagaaagggcacacaggcacagaagttggtttggggtaagacgatgtggtcagttcagaacacgctggatctaggcagatgcccagcagacagttggatatgtaagtctgaagctctggggagaggtctaggttggaggtacagatttagaagtcatcaacaaaaaggtagcagattaaatgataaaggaaatgagactatccggggagtgtgcagagtgagaggagcaagggaggcccttgggaacctcagcacttcaggggaaggtagaggtacagttgctggtgggaaaggcagagaagtagcaaagcaaaccaggcaaaagcagtgtcacagacgaccagggaggaaaaggacatgatcaaaatgttgagaaaagcagagaggtttgaaaatacaagaagcaaaaatgtccactagacttaaaaaccaggagaaaactggggggttcttgataaagcatcttagtaggatggtgagggtagaagccagggaagtgttggtgaggaagtgaagtcactgattacggactatgcttaaaagaatgtgggaatgaagggtggaagagagaaattagactgtagctagggagacataagcgatcagaggtagattctttctctcctgtgggagaatcttgcacgtatacacagcatgacgacagtgatggaagggctggcgaagcctcagggagactcttggaggtaaaccccatgaagggaggactttgtttcattcactgccgtgtccccagcacctggcacaatagcagacactcaatacatatttgtcaaatgtgggattttatcatttagaaactgcacctggctgtgagtaacaaaagtcagagaaaccgtgggtttcatttttctccccaggcagagtctggagctgggtcctccaagaggggtttggagcaccacaggtttcctcaagacccccaggctgccctgtgtttccctccttcatccccagcatatgcctgtcatctggtgacctccaaacacctgtgctgcctcctccagcacatccatgttgcaggcagggaccaggcaaagggcagaggggcctacttcaaaagaccatttccagaaaccccatcctatgacttctcctggtgtcttggttaccattgtgccataggctcaccctgtatgcatgggaggctgggccaggcattatgacttttagcaatattgcatagataagcatcaatctttgtcactgtgacgaagcctagtcactcagtgctaggcaaggttaatggaatgggttggtgtgtgcattattcttgaggtctttcttatgcttcatgttatacatttattaggacgtttaggcaacagggggataaaaatgaagaggagatgcatgctatgatctgaatgtttgcatcctccccaaaattcatatgttgaaatcttcatccccaagatgatggcattaggaggtggggcctttcggaggcaattaggtcatgactgggattagtgcccttgtaaaaccccagaaagccagcttgccgcttccaccatatgaagacacagagagaagatgccatctacgaatcaggaaatgagcccgcaccatgcaataaacctgctggagccttgatcttagacttcccagctgccagatctgtgggaaatagatttctgttgtttacccagcttatggtattttgttgtagcagccagagtgaactaagacagtgctgatctcgtattcttggagggaacccttagtctttagggaaagcaaagccaccatttggggcagggtgttctccaagtgctgccacatatgctgatgtggttaaactgcaaactatggtaaaaatgtggaggtctgtggaattgtcaatcaggaaaaagatataaaaagaagttaaagtcttcgtgcttctggaaggatatgtgccaaattgttaacattgattatccttgggtagagatgtggggaagtttgcagagacagttttgccttgtactttatataagtaaacagctactacttcgttgtcttaaaaaaaaaaaacagcctatgtgctcttcatgtgactcagaactacctaggcaatacgattaattgaattagtaaaattgagtgattatgaattttcaggaagtcattaatttaccacttctttattacatccacttctaacaggacttcaatataggggaatttgacttcaagataaaaagaccaaatttatttacccttttaaaaaaagacaacttaaaagcagacttgtcttacagaaccttccttagttggacatcgatgagtgtacagaaaatgcaatggataaaaagcttggtgatacaaagataaaaagtggggtcctgtccttaatgaacataccatttcatggagtatcaggtgtataaacaattataatcaatctgcttgttattctgataagatcatttactcacacatcaaatactgagtgcccaccacatgcccagcatacctagaagtcatccagtatgatttctgtctacatggagcatagagtcttacaggggagatagatgacaagtaaacaccagaataattaccaatggtgaagagcacaaggaaggaaacagaactcctaaagagagcgtggctgggcaggggtgagcaagaggcatagaaaaaggggcatctaaatctacttgggaggaagctgtttctcacataggtcatcatgttaggaatgagacttgagggatgagtagaagtttgccaggcaaagaaggaatgggggggaatagagagcagagctaggggcaggagacagctgacgtgtgagcagacataaaaagaagtccactgtggcagcagagaagcaggagagaaggcaagtgagggagccaggcaccagctcacagaggtcatgtgtgtcaaaacgtagtaatggccttctcttctggagacagtagggagccatggaagatgtttgagcagggaaagcgacatgactggattggcctgttgggtaactcagaccacaatgcattggaagggagggggctagaggcaaggggactggcaagaaggccagtcctttttctatgcctattttgatgaaatattctagaagggaagtgaacaaaggtagtcctagagaggaagaacaaaacagataggatacttccttagtatttgctcattcgacaatttatttttgcatatacactaaaaccttttttattattaaaacgttttattgtaggaaaaaagtatgaaagtagagtgaataataaaatgagctcccatggatctatcacccagcttcaactattatcaatatttggctgttcttgttttaactgttctccacctttttttcctgaagtttttttgaagcaaatcacagacaacatatcatttcaccatatgtacttccctctgtatctctaacatgtaagaacttgttttaacaaaatcaccatgctatgatcatacccaacaaaatttatcataatgtcttaataatacctaatacccatttcatgtccactttcccccaattgctacagctggtttgttcagatcagaatcaaaatccacctgtggccattttactgctatgtctctcaggtctcttttcatctctaataatctcaggggagacaggagggaggacgggcaggacttggggctaacttgcttatcgacacacagttttgcctacttgcttcctcccttcacacccactcttcttctcagccccacccttgtatggaaaaaacagaaattaaagtgctttgcccagcacccactgaagctatttcgaaggagtttgaagagtactcccggcaagacaaatgcctcggtccagtgctcaggtcaaagaggggagacgcttctcagtgatgtggtgtcaatagcagcttagttgttctttcctctggaaaattctacccatctgctttgtaactcccatacctaacaaggccttttatttcacaattagaaaataagcctgaaatatgaatgctgcctgagtgtacctacatttattctagagtttcagggtcaaaaagaatacaaggacctctgcatctacagccaagaggagaggggcaaagacacacagctacaaatgagaacctggctggtcaaagcctaactccacctgtttgtcagcactgatgcaagttaggtcagcccaatgatcatttaggagaactgtgctggcaaataaaaagcagaggcttttggtccccagatacttggatgagaattacaagtccagctggttaaaaggcacatgcccagtgctcacttcacacctactcaggaagcacacttgagttggaaaaccactgtctttacacttagaactcagtcctacatgactcctctaggatcagtgattccatcagttttgaaacatgaagcatgaagtcaaacaggacatgaccttggtttccagaaaaccagatgttcacatcagtctctggagcttggaggcagcacacctggggacttccacatcccctgccgaggtggcaaaagcaggagcagtggtgagttcacatgggctggggtttcctgaacactgctggcaattggagaatctgcaagggaacttctccgactcctaccagcagctgctttaaaataaaggtgatgtagctggtcaaatcctccatgagagagcagtgttgaatggaggaagagacacaacctgtctgaaaatggcacaaaggaagaaagatgtaaacaatgacgagaagactgcagtgtctacaaagctccgaggtgaacagatgggcaccccaggcccgcagcacttccttcagtctctgccagctgcactctgttttccttcctccaggaatcttgtttggtgtcactaaaacagcaattagaatcactttgaaatagtgatagtatttaatataactatgaaactatctgtgattgacaagtgcagcaaggagtcttggaatgagagcctttattttttcaattaaataaaagagttttttgtttctaaaagtaatcttgcagaaaagatcctgcgatcagaaagaaggagggggggagttttcaaacatataggagatcagactgtgcctatgtgtgtatatacctacaaacatatatatatttaaaaaattgttttactgtcaattacagcttcccacactcctagacagccgttctcaaggtatcaatctgagatcttggggaggaatattatctgatatgtcaccaagaattcaagaggtgagtagcctgatggtagtaattataatttcattatgtctttccaccatttaccccacttatgtcaaataatttaattgtatttcaaacctgttcaaggaaaagtacatttgatctttccatctagcaatttcaaagcacctgttcacatcccaaattatctgtgctcttaagtaagaggcagaaagaaaggaaccacccttctgatttcacatcaaaaaagaaatgccactggcaataagcaacttgcctggtgtggcataaatcatcagaagacttacagttgaatctaagtcttttcagtactgaggtggttcattattctgttacagtcttaaaattcacataaatatatactgccaataataatagcatacacctttatagcttacaggcactcttcttctaagtgttttacctatgttggcttatttcatcataaagaaaacaatggacttttgtgttgttttgtaaaaagatgcgcacattttaattaacatctgattgcacaagtctcctcccatatagaaatggattcttccacgcaatagataagaggtgctggggatatgatgatgaacacacagatttggtcatgaccctgtgggaaagagagatgggaaaaaaacaattctcttcaagtgtgatgagtgttacgaaagggagggaaaagttgaaacaggtttttttccaaacttttctccctccattattcgcagctgacttgggctccaccaacctggaaaactgcatggttggaatctgtctttataaaacgcatctcaacctgggccgagtatgcacactgatgtgggaaagttagagaagagcccattgtactaatgctcacctgctacagtgggagtctctgttaaacagtcttttcttcatagcattaaaaaaatttatatcactacaataaggttgaaattgatagagaatgtacaaacaatccccaaagtatatcaacactcttagttctgagtagaagttccagaaggcttcttgactgtctagatagcaagtctaatcatttgtgaactaagttaaagcagaaggcccagtttatatgaattggtattacaccatttgacctgagaacagccccttcatctctgagtgctttgactaaatgagcaacataataatagtaataaccccttacaagatgtcataagactcactgttgttgaagcaatttgagattttgactttattgaagcatagatggtgattataggcatgactcactgtgtggattctccctgggctcatcagtttcagagggcaagtgttggcatgtggacaaagagagggatgacacgtaaacatggcttattgcaatggggaaatattttcagtctcactgattgaatcctaatggttttataaattccccagtaccactgaaagcaaagcaagtaatcaggtgtgttttaggaataaaagcagcattattttaatttcgtattttcccctaaagcaaagccaaatggcattatgggagccaagctactggcagctccaccagccttctcctgagttctcggcattacagatctaccctcaaaggatgaggccagcaagcaccacagggtgcccacatggagaagagaaggccaccaacctcctcttagctggcacagaattgaaaaagtgtttttccaggaatggatacttcatctgttctgtatttgctagaattttaaaacgcacacacagacacacacaggcgtgcacacacacacgcacacacacacgagaaaaccacaaaccacacatttcaaggaaatggaagaattcattggtaaaattaagctaataagattattttccaaatataagaaactaaattttagactatttagccaaagaaatttgctctgatcttgcttttctacaacagaatcattccccaatcattttatttccctctttttctccccagtatccccatcttggtgggacaacagaacccaagaactggcttaacagtaaaatattttctgcatttgcccaaggacacattcccaacgaattcaaataaaggagactagaagaagagaggctatactacagtgctctaggggtcactctgtgatttgttgttgttgttgttgttgttttgagacggagtattgctcagtcgcccaggctggagtgcagtggcacgatgtctactcactgtaagctctgccccccaggttcacgccattctcctgcctcagcctcccgaatagctgggagtacaggggcccgccaccatgtccggctaatttttttgtatttttaatagagacggggtttcaccatgttcgccaggatggtctcgatctcctgacctcgtgatccgcccgcctcggcctcccaaagtgctaggattacaggcatgagccactgcgcccggccactctgtgattttctttaaggctcatcctagtattctcctagtccctaagtagatggcagtaggttttgttttttgtttttcgcagctggattaaggattgctgagaatatatggatgttttcttttaaatgtggaagtcaaaccaaacgttggagcattggcctcacagcagattatgactctagctgccttaaaataacctgaagactttgccttgccctagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaagtaagttttctgagtcctgcttataaattggcctctcatgttggttaagttgatggtttaacacttctaggtgaaaccaaacctggggttgcatctgtcttgtcttgctgagtggccttaggtaaagagacttctcccagaaagtccacttccccttgcagaaagggggcattgcttataagcaattctggacatgaaccacagaaagaactgaggcccacttggaaagggaacagaggggccatttcccactgatgtaattgaactagggctaagttcaagaggaagagaatgatccgcaaggaagcaacccagagttccaggtgaagctcaggtcagaagggccctggcaagtaaacacggctgtgggatgcttttacaaacacaatatcgtgaaaatctatgtgtgtagtactgaattacattccaaatggcaaattcctggcaaatcatcttccccacctttcactatttttttttttttggtcttctatggggtaaaggaggatggggtggggaagaaatgtaactggctgcccctctagttaaaaactgaaaagaggcagcaagggacatgccaaaagtagttggactctaagatagctacacacaacaaagcagctaagcagctaattgaagggaaattactgaggctcaagctgagattccaagcgggggccttgtttggcctctcagtccctttcatctgagaaaggcctcagttcctagcagtaatcagaggcaggcttctcagcctccttctcctaaagcagaataaaccacagggcaagtcgcatcctttgtttctctgatgaggccattactgagagtcactgtggcattttgctactaatgatgagcttgttattggtggggtacagcctattaatttaggttattcatcaaatcctccagcatggagttgaatgagacatgtgatgtggatacactaatgactatattgagttacaagcaatggggagtttctgtaaaatctgtcccttgtctcctggcagcatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgtaagatcccagctgggcttgccttgtgtaccctggacctcccagaagtgtgtgtgtgtgtgtgtgtgtgtgagagagatgtgccttcctggtagcacatctcatgtttgttttttgctaagtggactcttgcgtttcctcccccatccacagtcatcactggaatgctttgcttcagtgcccctgcctgggccctcccctctctactgcagcctacaatgaggttttctttcccattgcttgaattatatccctaatggaagggttcacaattctctgaatcctggctactcagataaagacagggaggaagggaggaagggtattttctcccagggggtccaaatctagctttaacgagggaggttctgagaaaataatatcatcaatattacatggacttctgagatactaagaaattagattctgtcagcccaggaagttgggagatggtgaattgttctgggaaatagcaatagactgagaaaataaaaacacttccttgaaaagcctttccctaacactaagtgataggggcagaaaagacacaaccaaaagttctctctcacttttctctctgttcgtgtctctgtcttgatctctgtctggttttaggccaactcaacttttgaagaactggaacacgttaggaagttggtcaaagcctgggaagaagtagggccccagatctggtacttctttgacaacagcacacagatgaacatgatcagagtaaggggggttggaggatggggaggggaggggaggaggaagcggtgggggcaagaaagttccacttgtttccttttcccaggaaagagttaatcgctattggagttagatcaaaatacaacaagcaggccccaaaggccttcattccaagcagtcaccaagtggggtcactgactttggatgagaaatatgtttcttgaattctgggagaagtctaaaagctgccacaagaccagtggcttcctggagtttcctacttttatgaattcactcaagggcctcaaattcaaagaggcatctccccaaggggccagctctgtaactccaaagatggtggaatgtgtttgtctggtctcattttcagctttgcaaaatgaagacaagagttctatatatcagggacactcaaaagaaaacaaaaatatccataagcaaaagaaagctttttatacaccatattcaatgacccccatctggcccctcctttgcccctacacatcttccctctattctagagacccatggacttggggaaatgggatatagataggtatgtttcatagtggaacaagctcaccagctcttcagggagccttagcatctctatcctcaatcactaaaaattagaaatggctgaagaacaagaccaaagatcctatggaatttctaagcagagcagtgactgtatttcttcttcccaaggataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggaggtaaggggctgcaagccccacagtgggccccttgaagatagccccatgagtggggccagagctcccttagcaagtcaagtggtcttgaatttaagctttcattttccccactgaagaaacaagaatccctacatcccctgtacagttctcattctctaacagcttatccatacttaaaacttatctatgctgaaaacggtttcctcttcacatctcctacttctcatgctgggcacctcctcctgtagccccctttaagcatctgtgtctgtcctcaaccctcttctgtctgacattgcttgagtggccatctatggccagtgtcccctcaaccccacagtccattgcttgctggacactcctgccctcaagttctacaagcacatcagcctcaacatgtcccctccaaaaactgtatgttctccttgcccatagaacatatccttctcctatatttcctatcctaattaacgtcctcagcatttgcccgaattctcaagtgagggatttcagggtcatccctaattttccttcttcaccctccacacagtagctgtcacttactgagtgttactttatgccaagtactgtgccaactgcttttacacacatatgcttcatttaattctcacagctccatgaggcttgcaccattatcattgccaatttgcagatgagaagccagggcttaaagaggttaaataagatcccacgcatgaccattaagaggagcgaacaggatccagctctgggggtgcctgagttcagagcctgcctttctgatttctcttaccaagctttgtctcctctccctcctaaatatctctcaactctgcctcttgcattccaggctctctgaggactagaggccttgtcatctctgcgccagcccattccaagggcttccttcctggaatccagggtccagcctctgttggcccaggcatttctctacactggcaccagagttacattccgcacacctgcttacgttgctctctcacttaaaatcttaatgactcgacccccaaataacacaggtcccttccaaatctgtcctaccccaccttcccagcccttgctcaactctgctacctggccctttcacgcctacaggcattcccattccatgacctcttgggattctaccctttgcaaatgctgttttcattgcccatttattagagcgcttttggtcacaagctttttgcttaaccaaaaagaaagcatttattggtggacataaataatgaagttcaggaggatccaagagttggaagccaccatgagacccctgtgtccttccacctcacttttctactcgcctctgctcagcttcatctctggccaggccctctcctctgctgatgccctagctgcttacagcccttagcagtcatctatacaccaaaaatccctttcccatagcagaagcaatgctcctagagagtttccctgttggtctggctcctgtacccacccctgtgtactctgattgggaggcctgggtcagctgcccaccatggggccatttctatgagcaggattactgtgaagtggaggaagatgtttccccaaaagaagaaacacaaggtagaaaagtgtatgtccaccaatgcctgaaatgactgtccctttcctcatctgctgagcttctactcattcattctttgagactcagcactcagctcttaaatgtcacttctgctttgatggaggtttagtcattcactcctctgtgcttcctggccctctcttcacacctctctcagacccctctcccagatagattagagttggctgttgacatgtccatctctggctgggcagctaaactggagttatttagaatcagggagcacatgtcagtcattttcaaattctcaacctcatactcccagtaaatgactccatctaagggtggaccactcttgcccatgggccaggtctgggtctgtgtcatctagaactgttggaaggtaggggcttctgtgagcagtaggagagggaataaactcgagggccctcgggagcatgccctcttgtctcagacttgtgagtcctgaggataacaaactagtgaagaaaagcctcgttctatctgtcacctggtgctcttgaggactttctgttgccctggtgccaccacaattttccagagtgtgtgaccctcgctctccaaactctggaagtggcagccgaggctccccagtggcctttcagaaggtgccagtcatgacagcagcaccaaactgcaggcaactactaagcgatcaccaacttgtctgaagataagaatgaccttgaatgcattttataaaacaggattttttttttaatttttagattttctttctttattttaccttaagttctgggatacaagtgcagaatgtgtaggtttgttacataggtatatgtgtgccatggtggtttgctgcacttgtcaacccatcatctaggttttaagccccacatgcattagctatttgtcctaatgctctccctcgcctcgcccctaccccaccccaacaggctccggtgtgtgatgttcccctccctgtgtccatgtgttctcattgttcagcttccacttacaagtgagaacatgtggtgtttagttttctgttcctgtgttagtttgctgaggatgatggcttccagcttcttccatgtccctgcaaaggacatgatctcattcctttttatggctgcatagtattctatggtgtatatgtaccatattttccttatccagcctatcactgatgggcatttggattggttccatgtctttgcaattgtaaacatacatgtgcatgtatttttatagtagaatgatttatattcctttggttatatacccagtaatgggattgcctggtcaaattgtatttctggttctagatccttgaggaatcacactatcttccacaatggttgaactaatttacattcccaccaacagtgtaaaagccttcctatttctcaacagcctcaccagcatctattgtttcttgacattttaataatcaccattctgactggcatgagatgatagatacccatttgtcagatgggtagattacaaaaattttctctcattctgcaggttgcctgttcacgctaatgatagtttcttttgctgtgcagaagctctttagcctaattagatccatttttcaattttggcttttgttgcaattgcttttggtgttttagtcatgaagtctttgcccatgcgtatgtcctgagtggtattgcctaggctttcttctagttttcatgattttagattttacatttaagtctttaatccagcttgagttaatttttgtataaggtgtaaggaagggatccagtttaagttttctacatatggctagccagttttcccaacaccatttattaaatagggaatcctttccccattgcttgtgtgtgtcaggtttggcaaagatcaggtggttgtagatgtgtggtgctatttctgaagcctctgttctgttccattggtctatgtgtctgtttacaaaacagattcttaagcatcaacccagatcgactggctcagaatttccagggaagaggcctggttatctgcatgtttacagacctattagatttgtgggacctgcagttcccttgtacagttagttactcaattaacatctccctcctctcatggtgcctctacctgctaagcccttattcccagccaggcccaccaccatccacccactgctgttataacataagcaggacctgtgcgagggggtgtggacggaggagagaggctctgttgcttcatttgtgcagcatggagttcagtggttctcacaatgtttttgcaaagtatataaagaatactccttgtctacttgacattcgtatcgtgacataaatgtcttgttttccagaaggattattttttccaagcagcttgttcctaatgcagccccaggcaccaaacagatacttaaaatatattaattgcttaaatggttaagaattcagtctctggacccacactgcctgggttcaaattcctattatctgtgcccagtttccaagtctataaaatagggatattaatagcacttacctaataggctcgttatgagaattaaatgagctaattcatgcaaagcactgacatatagtaagcacttaataaatattagctttttaacaaaatacaagccaaaaaacactgcttaggagaggaaatgatgttagtgcctcctgtaaataggcccagcctccaagctggtgctcctctaggaatcacaacgctgcaaatcacatcctccggggccgccaggacttcacgagggcctctgagcagaggggtatgatgggagcagaagcccagcagctgtgatgatgtggtttctgatcttcctgcccttggggtgggggaggaggaaagcaaggggcaatgaacagaaaggagaagatagcggggaggaaatgtgtgaggaagaaacacatcactgtggcttgtcctggatttttctgcttctgttctcgtgttttgggaagtctggaggagacttgaaaatcattcatgtccccaccctgaggatggcttagtagcagagaggccatgaaaactctttgctgatggctctgaaagcaaggatgttgcttcactgggctgctgaaggcctgcctgggggttctgagcagagagtacaggcccctcccaggagggcggcctaaccaccatgctggcatttctgtggaccatggtctgctgtctcagaccccctccacaatagggtctgcaatctcattcaccccataaatacattctgtctttcctctgatcccctcccattagcagggggaaataaatggaagtcagacggcccagttagaaggcaggcagtggagtaggaaaatagatgatggtggtttggggagcctcacatcactcatggggagacattcattcccatgggccttccaatcacccttttctccaaatctaaggacacaggacaaatgggtcctcatacaggcaaatatcttaaactggtatgtgtattcatttatagttctaatttatatgtgtctttattcacatatattttgcttctggagaaaagctcaattagaaaaattaatacattattcttcttattgcccttcagctaaaacaagcatacacacccctcccctttggattttttgtttagcaaaaggttaggcctggcacagatgaaatactattcagagttcacagtgtattttcatttcataatatatttgattttcaggtcttgaatttcacatcaggaagctgatataggaagctgaattcagccagattttaatacgaaaatacctctgatcaaggcataaaattgtactttaaccagtaaccactgtatttctctaagctgtgaaaaaacatgcattcattaactgctttttcctctgctgtcaacacagtcaatacatgtgcataactccttattgtctacatggtgattatcttgctgatgaattctcaaaggccagagatttggactattttttctctgtaaccttgcatgttcctggccacatgccaccaccacccaaacagaatgtacgcagggaatgtatttttcaggataacctaagaaaaaataggattaagaagataaagctgctgatcatgtaatgtactttagactcagatatataaatatttgtgaattatctgtcctatttctttcttctattaattcattgactctagatgtgcattggaaggctagggagaaatcaggggatcgtgagaaagagcacagaagtctgcatcacacaaacaatattatttcaagagccatgaactagatcctaagcaactcataggcaatgacctcatttcatacctctagtctctaagaaacatataactggcctgaggaaggaaaatgtgggcaaggggtagaccggggtcatgggtggaggtccaaatagtaatcaatggagctcatagggtggactgatattgaagctgctatgagccagccacatgctgggcactgttacatgtcatctcatgcaatactcccaattacctgcctagtaagcataattgtcattttatagaattaaaaacagactcaaagaggttgacagtctaatgtaacacaacagctaaatgggggatctggaattataatccagagctgcctggctctgatgagaaagctctttctgctgtcatatgcagcccacattaatagggggctcagaaagtattctctggataaattatataatgaatccaatgaaggaagacattattttataatatgcagcataataggcactattatgattggattttcctgcttgaaagtagctagattagagtaggaaaccaaaaagatgtgaattcattcagtcattcatgcatttgcatggattgagctacctacatttgaataaatgctgttaatccctgattccttggaagctcacattggagagataagcatgtcattaaataatgccataatagtggtatctcagaggactagcagaacataattcaatctgacagagtagaaacagattgtacaaatccaattcaaaacatcataaatcctctaagcactgtcaattcttcctccaaattatctctgaaattcctccttctttcccatttatggcctccatttacagaagcgtgtactgtctctcttagctgtttgccaggccgccagtctcttgctgttcagctctcaactgcttccagcaagatctttctaaaatcccaggcttgccaagacttagcgcccacagctccacagtgactcctcattgctgttaaggtaaaggccttcccagtctagcccttcatgcttcttccatgttctatgggactgccccaggcttcccacctggtaccactgagcctttccatccttcccccactcgactgccaggtcaacacccacacccacgcttcaggactcaggtcctatgtttcgggccttcttctgtgcaccattcccttccctgtagcccttgatcatgatttgtttatacgcctccgcaccttcatggccctgaacccctcaagggccgaaactgccttacttttctttttgacttcccaacttaccttagtggagctgtagtcacatagaatagacgctcataaatgcttctctgggctgtaaaggttgaattttccagctaagcaaggaagaaagacaatttcaggcaggaggaagggcataagcaaagtgcagagatgtgaagctcaagagaaatggatgggctgggcagaggtgtggctgcagcatcaggggagaagaagtagtgcctggagtcagcaggcacggcttgcaaaagcttcacctataggtgaaaggacaccatctcttgcaccaataggctctgtgattggaggcaactttgctgttttactgccagaaaactgaggatgataacccaaactgcagttcaagtggcattcactggtgtggctgaaatgggtgtttgtggccagaatgtggtctgattggtcagtgcccagctctgttgattagcagatgttttgaatatagtagcatccatgtgcccaagttgttgggatgattcaacaagaaactttaagagctcaagtgccctgcagttgtcagccaggtgattctcttcctttggacccagttagacgcaggcattacctcgtggctttgccccagtgtgaatctttgtcctccaacttgatctttttatttgtttcattattgtatttaagttgtttattttagagacagacattttttaacagctgtgcatttcctgtccctttgttttccagtcgtcatgtgtttccttactctctgtgggtgaacgtttcagatgtctgtttgcggtgcccagcgtgcaagataaaatttattgcagtgccttcggcctctaactcaccattccaaccaattcagatagcccaaggctgttttatccagtggatttttccatgtagtgggaaataaatcttgaatgttactgtttagattagccaggaaactcattctgggatgtttgcccacatccattggcatttctcaaaaggaaccccaggtgtctaccttgacaccagcagggccacttgagccctccgctggcattcatcgcccgctttgttctcagcctgagtttaggagttacagatgtgagaggcgggattatacagccaacatctctaagcgggcagtggctcccttaccctcgaagacctcactcctagcacgtcctggatgtattcgtcaaaatatgtcctcttatgccacgtcagcacagggttgctccccactttgatcatcaagtttaaacaaaaggaaagattttctttctttctctgcctctactggacatcatttcccacctaacagataatttaatgtatctgttactgaatgtgtttgaattacagacagagaggtcacagttaaagaaggaagcctgctgctactgcagcttgtcctcccaaggaggtgtttgatttagctgtgtaaacaaatgactgcattctccagaggtcctgaacacagctgcctgcgctggagagggctcaaacctcttccgccagggtgaactctgcttcctggtgagtgccagcaaaacaaccaacaaagagctgtaggacttgtgtggacttcaaatggtggtggtcctgccacttgggctcagccacagcagttaggaaactaaaggggaggaggaaagccctttccttgctttattgtcattggctgtcatagggcattacaatggttctctttgagattctgagctccggctataacatttgcccagaatctgcctctgaggccttaagacactgtgtttttattcagcaaagatgccctttgactccttttcccactagtggtgctaggtttgagcaccttacactggccccttacaatagccagttcttgtctacctacattcttccctaacattcatgattgcatagttactcttagtgtagaagcagacagcttttacacatagactccatggccgtagcctcatagaacctactatattctaacttgcaagctaatcagaccaaatatatcaaaatcaaaaacctctgctgagagtttattcattcatctctgtctcccaaacgtacttatgtacatacgtgcactaatatacatgtccattagccaagattttgatttcagggatcaaagcaagtaccaatagggaatgaggtcacttgctgcatggcaggtggcttccccatgagaatgcaaggccacctcatgactcatacttcagagggtgacccaggaacttctgattcatgtccaaagcagcttctacaattgctctaccttgatctagggaagatgtggggaggatgacattcgggattagctttataaggccttcctgtgggcagagttgtctgactttcacctagtgatcaacaagcagctagcaagcatcagtgtgtgaggccccacgccctctcagctcccctactgcccacctgggacatgggctttggcatctgtccatagcattgttctaaccaaatgaggtgttatggatcagctcaggatgggatatgttcccagacatattatttaaagaaaatagctcccttcctcccctgataaacagctgccatggctaaaaggtaacctggctggggcttaaaagtctgttgactttcaagatattttgcaaaaacagtcataaaaatggtatttatcagatcctaactatttgtgagacggtttggtataccatagtggttaaaaacacaggctctttccagaggaggtttactttgcttagtcgtgtctcctaagtgaacttggacctcataaggttgttgtgagaatgaaatgggtgaatatgagtaaagtccttggaccagttttggccgtatagtaagccttcagcaagcatctgcttttattcctacagggaggcaattgtaagcccttcacaaacagcgtctaatgtgatccttagaacaaacctatgagatagggcatatctcaattttgtaggtagggaaacagaagccacacaattaggaaatggcaacagatctgttagactcttaaacactatgctacaccaatttgcaaggcaaggaagacaaagcacctttgaaaatgggtcagatgttttagggtaaatgaacgtttgagaatcttttaagttttttttcccccagagattatcaaggtatcattgtagggggatgcatcaggaaacatgactatgaatcagctgcctgataaaccagccaggatggagcccacgtcatcacagcagtcagcaatgccactgaaaaacatcagctgcttattcccgtatagatttccccttaagacatgaaaagggagttcaaagagaatgggccagatatctctgagagtcatattactaaaatatatttatttttactagcttttttgttttaagaggtatactgtcattagcactgtagcaaaaattcacgttttattaatttctcctagtttatcatgtgattctagggtaggatgcagagttatattcaaaatacacaaatcaactcaactcagtaaacatatatcgaggccctatcatgacaaaatgctattctagagaccacggcgaacaagccacggccccagcctcaaagaatgtactatctttggaactgtgctggccaatacagtaaccagcagccacgcagggctatttaaatttaaattaattaaaagtaaaaacacaatgcctcagatgcattagccacattttaagtgttcaatagatatttgtggctcctgcctgccatattggacagggcagatatagaacaattccatcactgcagaaagttctactgaacaatgctgctctggagcagaagatcttcttgttcagggatgttacacccccgcttgtggctagagtgtggcttatcctcagagcaaggataggggaaccatggcactctgcaggctcagcactgaagacacggatgcaggctctgcttctgacctagattgaccttgggcaaggccctttgctcctctgatcccaatttcttcaccagccaagtaagaacatcagaccacaagccctctagggctctgtccaaatgccccatgactgagtgaactggtagaacattctatgtgtgtgtcacaacatgaagagcaaagactttcatctccccaaataattttgtttttcgttttaggaattaaatttcagattcactctaattgccaatactaaaattctctatatgcagttctaaacttgacaaaccaataaaaaaagattatttgactacttatctttgtacaacattgaggtctccctaaagcaaatttaaatgcatattttaaaaatgtattctagcagttcagttcagaagccccctggcccaagcatcacactgtcaatcctttgtcctcaagcagcatggttgggtgggttaagtactgacaaacactgggtgtcaggcccatggtcagggactgtgctaacagtctacatattagatgccacctacccccaccctcaacagacccaaactatttatccaatagcaaaccttgcattatttctgtccagaagaaacaaacatttattgacaacttttggtgtgtgacctgtttaagtcctacatctcatttaaggactggtcaatgttaggctaggcaatgcctgtttgtgagagaatcactgcctaaagaaaattctccatttcccttagctctatggtgggtgactacacatactggtatttcttaaagaaataccaattccatttccttttaacataattattaatatctcattagcatggtgtcactgaagcctgggcccaaagaaataccaattccatatcattttaagatcattattaatatctcatcagcgtggtgtcacttaagcctgggccctttagaatttttcatgtacctgtgttcctctgcccatatcagctggaacactaatagttttcttcctttttatctagaagactgagaacattacatgggacctgcccccagggcatggaggctgaggtgggacagtttagttcaggaggcccaagaagtgttgggtgtgcagccccttgttcaaacacagcctctgaatcgccagaggcttccggtgcatactctgaggcgcaggtgggactcgggagtgagaggtttcggcgaatgaattgggattgcctacttcttcccagtgcagtggagcttggttctgtggtcaggtccttacgccctgtctgcctttctcgtttctttatttctcgggtagtagttgtggaatcaaatgacctggggtttgatacctactctaccacgcctctgggggagtcactcagactcgttgaacctaagttccggggctgccaagtgaggataagtagtaattgctgatccacctacttgacaagatagtagtgagggccctgagcgccaggctgtggatccagcctttcccacggttcctggtgtggcaggaagaactctaggcctgaaggtgaaattggggagggagtcccagctctgccactgtctctctgggtgacctcaggcaggtctcctcaaaaaaataagatactttataaagctcagtttcctcttcagtaaaatgaggattccaggtaactcacagatagtttgtggggatgaatctgttccttaaagcctgcagtacatcaataacccagtcttcctgcttgctttcccccctctccactaccagtgatcatagtctgatcccataggtgatatcccagctcaaaaccctacattagcttctgtggctgtttaaggcctgcccagaactcccctggtcttagcactgaaagcacgtgtccggggaagccctgcattggtcgttcatactactgagtcccgcagggcaaaccgtccggtcccaccctcctttctagtgctgctgtcacactcacctcccttcaccctacactcccttctgtgccttgcaattacctagggagttttttacaagatatggatgccctggccctgccactagagattctgatttaattgcttggggtagggcctggcataggtatcttttaaagctccgcagtggttctaaagcacagccacagatgggaaccactgatctattcttgtaggtccccagatacctcatgtgctgttccctgtgcctgagctgacctttcccccactttcctctcctcggctaattcctgcttatcctcctactcaggaggctcttcctccaggcagccttccctgatccctccaggaagacttagctgcgtccctccgctgggcttccccaatacactgggcttgctttcattagaacctgatccttccacattatggttgttggtttgctccaatcctctccctcattagctctcaactttctttcaggaagagatgtttatctttccttcttgtattcctagagtcgaccaggctctggcacattgcagattctcagtatgcattcagggaacaacttaatcaagacaagaccatctgacttcttgtgagttacatgctaagaaagaaatgtcgacaccaatagccctcacaatgataggaacaggaggttaaagaaaaggaaatagatgcaaatagcaatataagtgctttaacaaatctatacaggaggacaaccatcatattcaaattttcaaacattcttagttctgctcttttgtgggtaatggtttttttttttcctcttccaggagaagaaaagaggcatattatagaaattcctcctcccccagcattacttgtcacagaattgtaattggaagtgatttccctgactaagttattttggctgtctgttattttctctcttcctccttgctcttccctcagctggccatcctgtgtgtttggagagagccagaaaggttcaaggctaggaatgtttctctctctctttaaagctctttaatcgtcaggctttctgatcttcaaagcaggctgtagccagtgtgaccccactccctcgcctccccatgctggagagtaaaagcctggagtatttttgtcattttgaagacttgcatatttggacagccttggacatctggaaagtgtggtcctcactagctctgcagggataagagcacgtcagcacttccaagctctctggcgcccctacatctggacacgttgaaaaattaacaccagactctggagttaagcaaacattaagtttataggcctccttgcatttgaccatttcctgggacagcagcccttatcctgtgactttctgtgtgtagagttgagtctttgcagttggtcctcctcacactctctcaactttgtgactctctgcagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtgatgtttcaggaagggctcgctgcatttctccaaagtcagtgggaaattacatttggtagagagaaagggattgagactggactcataaatcaataaaattaagttaaataagaaaaaataagatattttataaagctcaacaaagagtccttgaatgaaagcaattacagagtcacattgtggctaatattcaaaactgagatttaaactgaggactaggaaatagaattggatccttttgaagcgtttaggagaaagattttaagagaatgagttccgagtcaccctgtggtcgggaggtgtgagtgagctatccaagcccgttcccatcctttgtccctctgtgtcttctcaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattcgtgagtctgaagttcgcgatcctcctccatgacacgctaatgggggtgctggagtgggctggggtgggctgggggtgccctcaaggcttccatgtctttagagagagccccagggaccagagccaaattggagagcatggagctctgactgaggaacctgcttctcccaagctccaggcaggcacagatgagtcagtgcagtggtgggaaagggaaaagagttgatgttgtagctggaaaagggaaggggaaaattaaagcaaggaaagtgaggctgggggaggggacaaattccccactatgtagtatgtttggtatgtggaagggttctggtcagaatgtttgcccaatgattgccacatcagcattcattttggactctgtatggccagtaggtctggttcctgggagccctggaataatgcagccccttccctaactaacatttccatgatgtatgctcaatgacaaggcagaggaatgtgttggatgagctcaggacctgcctccctggacactcccatcccaggcctgtatatctgttgaccaggaataagccaagcaagcagcctactgtttgactgaatatggatttggggggtggtagagaaagggccggggtggagggttgggaggctcatttgtcattatagatggggtcagacacactaccaaaacagcagcagagatctacaattgagttcacctaaaactcagtgtggacacaggaaaccctcttttaataactgtccaatgggttttccagcctcagctctacagaaaacttgagataacagtggccagtctgcagttagtttgggttcggacaataggcagagctgggaaatggagccaggggcgaaagcccaggtccactttaggatcaggacgggagtggctggtggggaagtgaggtgggtgtggggaggcaatagggagctgggtcatttggtatgggagagtcctctggtggctagtcccagaagtgcatgctttacgaacatatgcttctctccctagggccaccttgagtgaaaccctcccatgctggaattgggccctttcagtgacaacacacaacagttttcaatagataataatcccaagggctttactagcacatgaaacacagggaaaacgtgtaaagttcacaagaaagtcgttccagtgtatcaaatctatcctgtttgccaggtggatataccagggtctcctccacctgtgcatggctggtggtgggtccagtggctgttggataactgatgtattgatggatcattcgccttctgaaagtgccaaactgattagttattttgtgtgtctttttgtgtaactagggtttgaccttccagggcagactgtgctggggcggctgaccccttggggagccaagttattgctcttaccaccaccacttgcccttgtcagtcctccaccctcttgggtttcagtgtcagcatgtagctgtctactcagatcccatccacatcatcaagtctgcagttttttccttgcaaggccttacagggaagatctttgacatagaggatataattttattgacacattttacttgcagagcattcacccgggctaaccagaaagccagcactctgctataaacaaaaaataatgcttcagggctaacatggaatgtgttaaaagattccagcccattaaatgtccaggggaggttttcctgttttcctttccctccatctgggctttgttctcaacacattcattcaacaaacatttattctgcctctaccaggtacagagcactctactattctgcttctctccttttgctttagtttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatggtaagccaaatggagaaggcccagaaaatcttgaatactttggttcctttcccctttcctcctgttcatgtgcctggattagtcatgtggccaccaaggagagcgtgacatctagcttcccagcccttccttttagccaacgtgggagacactcaaagagacgaaatctcctgaaggagccactgtatcacagcatcctcccatctcccacttcctgcccaggggtccatggtccacacagacttcccagtcccattccgtgaccatctggagaagctgctattagcagagccctgcacagggtgatagtgtaattaaagtggtcttctctttccaaacacagaaaaaatcagttcagggagtgttttcctgggcttacaattttaactactggctagagttgaaatggggaaagccttttgccttttcagtagcagtaggggaggagatctggattatttacttatcatcatcatggtcacctcctacatggcttcaccaaaaaacattctgctgcctgaaaaagctccaacacctctctctcttttaaaggatggaatttggagtccatccttcctcagtgataaggagtttttatagccacaggcagcatctattggtctgtcctctgcaaacttgcaactcctctgagagctagacttggaaatgaaacattattttgcaatgcgctgctatccttcatttttagctcctccaccgtagatgatagtttgtacttgttaaatgataaggatataaatttaggtcattttttatattttattgggtggaatttggtataatttttagacttcaggctttacaggctcctgagatggactgattgagcttgttctacttcttccccatcatgataggaagtgctgtaccacactaggcagtgtgtgtagtgaccacagactggctgagtgtctcccatcccatgctggcccatatctggtacccacctgatccacaaatgttccatcagatcctgttcaaacaacacatctccagttaagccaaatcttgccctttctccttacggtaaaatgtactaaatctgaaggttttgtctttttaatgttgctccatgatccagtgatctgtggccttggttatgctctgtgctagagtcctaacaagacaaatgctaaggtagaggtcattctgctcaaacaacctgaccccacctggatgtgggcttacatttgcaaagggcaccaaagttctaagagatgaggggaggagctgagccccttgtccttatctaggtttcccttgttctttcccatccctcagtctgcttcttttcccagtaccaacatgtttgtgtcctcagaattaaaggagtaaaaatgtgtaaacatctgactagcaacagccatgagattttgcctggcttgttgataagcagcattgagatctgccctcctaagaatgggccattaggtcttcaaagcttttacgatgtgaggtaaagaatgttcaccaggagtttcatgcacaaaagggtttctctttgtgggaactagaacattgttccagtgatgacggaaacagggctttccataccaaaacagggttttcctttgaatgactctcccacctttcccttgtctcttcctccccacctcaacaacacaggaaagaagctggaagcagggacaatgggaaggtccctttgttactcgagctattagaaacaaaaagaaaagtggccatctgaggaagccacagctggtgaaactgtagggtcacagagtgaattacacctctggcttaagtcagtgaaaagtcctagaagtttgtggtcctagaagtcctaaaagtttatgggactttgttttgagcaaggataagaaattgatttcaggctgggcgtggtggctcacgcctgtaaccctaatactttgggagacagaggcaggtggatcacttcaggtcaggagttccagagcagtctggccaacatggcgaaaccctgcctctcctaaaaatacaaaaattagccaggtgcggtggcacatgcctgtagtcccggctactcaggagactgagcaaggagaatcccttgaacccaggaggtggaggtctcagtgagctgatatcatatcactgcactctagcctgggcaacagagcaagactctgtctaaaaaaataaataaataaaaaagaaattgatttcattcttctgagaactgcaacaactaccttaaagtgattccatccaaaacccacatgttcagccatggacttgcttttatggagctgcgtgtgggtgacacacaaaatcaggagctctgagtcctaatttagacttttatttagatttcctcaaatttgggttccagttaagcgtgggtctcttctgtgccccgctcccctttgccatttgttttatctgttcttcagtctgttctgtcagtacccacaggcaggagagcagaaaggagaaatggcagccacagcagacaaatggcacattcgttccactcagctctcgcatgcccatcacagatacagctcattggtctcttttctatgagaggaagccagagctccagggaactactgccaactgatcagaactcatttaggacatggacctatttgttcctttatgttcctgggaagagcacaggatgaattctatgtactcatttacgtgttcagagagtaaagtgcctcataggatgcctccagcaaaagataaccaagaaggtctaatacctttgacaatctcagtttatcctatagtgtaattggatagcagttcccctagcaaaagttgctagtttggtcctattttctacatagccaaagtgattgattcattggttaatgtgaaagttactgagtactgccagcaggttctaggaaatatatttgtgtgatattcatggatggggaggatcaatccacttccaagtgatttggattaattactggtattttcacctgtgtgggtagcaaacctcagaaaatcaagtatagatgacggcataggacaggccaggccccaggcaaaatgttgaagctcctctggagttccctcccatctccctcttttgttttccatatacctggtttatccagggccctggagatgctccaagaccccctacccaggtcttcctcccttgtcccagctatatttctccatattaccactcttctcaccgaggatttgcttacttaacacataataaatactattaaaagagaaacttaggcacattaaaatgttagagttgattccagcaaacagtgattcacaggaggctccagatcacaagtggttcagggccccactgaggggtagggaagcaagacaaagaaaaacaaagcaaatatttgattggttcaagtggaaagtccctgattacaggttagtgggcagtttgtgattagttaagtttctctaagttgggttttggtttgctgatgtaggaacacagaatgctggggccgtttcaacctaatggtctcccaattaatttttttaacattactgatgactgttaggagtctaatgtgctactcctcccagggaaaatggcattcctaggattaaaggaactcagcacatggagtgtgcgtagaaatttagacactaactgcaggctggtgggagagagccctttagggcagaatgagaaggcgtccggccaagggcaggagttactgacgcatggcctcttggtttcagcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtggtgaggcccttgggctggcccctgtcctacaacacgtttccttggaagggtccgtagcagtcctggaggcccagcctgccctctgagggggtccactttgcctttgacctaaggttaaaaagttcacgtgaggctaaaatgtacaggggcaaaagtgggagcagtcctcaccccgagcgatgcaacagtgactcctcaccacgcctgcttgattcatctgccctggaaagtcattaaaaaaccagttcaactcatgggtccctttatttactcacaagagagagccagcagcccatttcactagttttcctttcctactctttgagaagaatcagaagggagggagcttgccactttactatctgtctaaagagatgtttccattaattaaaggtttttgttttgcttcaaaaaaacttgaattggagtatttccacaagtatctttaacatgctctaccaatgtttgcagaaagaagtgcagaaatgagactgtccacagagtcaggctcgctggccaggagaggactcccgaagctgacttctgatggcctgagaaacttcctagttcacaattcccagacccagacaaagagcactgtcttttctctaattgttttcaaatgggccatttccaccctctaatcagcctctggccctggagggtgcagttccccttgtcctccggagtctccctgtctctgtgctgtagagtcaagaagggacaaccacctgccctcactgggaaaagacagaaagtctgacttgttctcacgactcacacttattaggctccagaggtgtcagggcatctgcctttcatttcttaggttaaataagaaatcaattgctgccatttgtagtacccaattttctaaaatgatcacaatggataagtggcaagaaatccttatgactcatctgtgggcagagttgggctattttggtaatccttgagtaggcagatggaatttgaggccatcttcttgggtacatagatcactaggaagctataggtctagcaactgtggattagggctgggctgagaattgtttcatgttttttgtgactgtatagctagagactctcttgtttgcagagagacactctgaactccccctggccgtcaagggaaagactgccttcaccctcctgagctgaccttacactgagagacaatggggaccctcttttggccctcccctctacctcgagggcatctgggtgctgttgcattggataaaaggcactgctctttttctgtgccctctccgcctcactgcagagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggtaagcatcctcctctatagggtaaaggtaattgagttcttcagatccccagccctctccattcatctagtttaaatttcatttcttccaagctctttgtcagaaccagcatttgaagtttaaatctagaagttaaaaatccaccagcaaatcctactggctctacttgagaaacaaatccagaatctgatctcttgtcaccacctccaccacaaccttcccaatgccagtctcttccttccactaccacctcccatcagtccattctgcacactgtattcagggagatcctttcagaatcaaggtcatgtggtgtcagccctctctgtcaaatgcttgcactggcttttcctctctttcagagtaaaacccagtgtctcaaccctggcctccaagctgcttcattatccggcctccaactctcttcttcatcttacgattttccctactcctccatgttcctctgctccagccacgtcggcctccttactgactgtttaatacaccgagcgcatttcctcttcagggcctttccacctgctgttctcatgccagaagcacatttctctccccacaacctgcaacccgcccctcatatctgcaggcttgcttccttactttgttaaggtctctgttcaaatgtcccattatcacagggatctttccagactgaagagatctacataactatggctctgtaaacaacattcctccagggttcctgtccccttaccctactttattttggggaacattcttcaccatctgatacaatgatgtatcttatgcatgtatttactgactctctgcccttagtagaatatgagcccagagagcatgcatgtggtctattttgttaactgtgacagtcccagtgcccagaatagtgcctgacctttggtgggcactgaataaatatctaagtaatctgtagcatggaaaatcagcttctgaaaattggctgtttgcacggtcgtgtatttgcttggtagaaaatcaaattttccttcaaattagcattttctggtaactagagctgccccatcttcctctgagtggtctccaagtcagccaatagccttgtgctgtggcagccatgcctggctcttgatgctgtagccaaaagcaggcaggggatggtgaggctggtccagtccatggggagggacaaactcacagctctcagatcatctcagggcagcctttgttggcagaaataggtaggcagccaccctgaataggaggaaggcttctagactgggtcaggaggcctgggtttgcatcctagtggcaagcgtgcattcatttactagggctgccataacaaaataccactaactgggcagcttagacaacagccatttatatctcacagctctgaaggctggaagtccaaaatcaaggtgttggcagggccatgctccctctgaaacctgtaggtgcttgggcactccttgacttgtagatgcttcctgctgatccttcgtctgcacatggcattctgcctgtcttacatggccatcttataaggataccaactggattggattaggtgcctaccttgctcccatgtgacctcatctcaactaatcacatctgcaatgaccctgttcctaaacaaggccacattatgaggtacctggggttagcactctggtatcttttttcttgacagcacttctgacaccaaatgtgtgttttggttttttgttgttgttgttttggcaccaaccaattctcctatattaatgggttgtccaagaattcaattgaattctgacactatccagaattcacacagactccacgggttcagtcccacaaggcttccccgtcttcagatgccagctggaaatgtggtgcccaggctacccacacttttgccaaaatcctgtacttacaatcacagctttaaaatgaaggatgcagctcaggaactgccacatggaagagaagcacagtatggggtcgggggaagagtttctatgctctctctagacgcaccactctcccagcacctcaaagtgttcagcaacccaaaagctctccaaatcttgttgttcgagagtttttataaccctatctccagctccatactcccccattggaggttgagggttgggactgaaagttccattcttcacatgtgtggtgtttctggtgaccagtccccagaaactgcagctatcttggggctctaccctgagtcacatcattagcataaactcagatgtggtagaggaaggggcttattatgaataaaaaaagacactcctttctgccaggaaattccaagggttttaggagatctgtgccctgcacaggagctggggacaaagaccaagtatattttgtgttatgccacagaccccaacatgtctttttggagggagaccaaattcaacccatgacagtgactttgaacaagacatttgaacttagtctgttttttctatcctactagattgttggaaacagatataatagatgaaaattagttgattaaaattgaaatttgtgcataattcaaaagttttattttagccaagctaaagctttcatttattcaacagctatttactgagcagcacctgtgcatgaggctcagcagggccaggttctgggaacagagcggtggagataaagatccagacctgccccgaggaatagacagtccagtggcagcaaaggccatgaaacatacggcaactcttaaaaaaagccgagaccatgattttacaaaatcaacattttgtagggagcagaactttcaaagagaactggactagaaatttgggagtctttttcttggaaccctggtagatccagtagaatgagggatgggggtgtagggttaaaaacactgacattagaactggattacctgtgttggaattcctacatttctgtttcactatctgtgacggggggcagatggctgaatctcagtgtgcctctgtttcctttctcacaagaataatattactacctatctcctggggttgttttgaggtttagattatttaacacatggaaagcactcacagcaatgcctgccacagaaagaatatccagtacatcttagtgatgatcaccattattattatctgactcctggaaaaggacttgatttaattctctcatgaaacgttttcttggaaaactgatgtcaaccaagattattggtcttgctgttgcttataacaccccaaaaacatgactgtgtggataaaaatatgttggaaggggtagtctttctgggagcctgagaatagccatgtaataataactgcaaatatctatagttacaatttgaggttcaggtaaataaactctagatcttatagaactgcggtaaggtaggatagggagactccttcgactttctctgtttatttgtctctatttttaggagactatggaaccccacttccttggtactttcttctacaagagtcgtattggcttggcggtgaaggtgagtcctttaaaacacaaatcttaatgtttgaaatcaactccttgggctctgtgcaagatgtatatggatcacagaggtggccctctatgtaaacggtgtgattcctgatgagtcagctgcctcctggggctctgccccttgatgggcattgcagcgtctgggggaccacctttcacaagttgctgggccctgtgtgatcatgaatggctgatcatggatgaagccctgggtcctgtacaccttgtccagtagactaaattgccctatttaaaaaaggccaagccacttcagggttcaaagaacttttgcagcttttcagtataaagcagaaatccagggaatcatgaaggaacctttgcattcatctcccattgccttccttgtgcctttttattcttctctgccttttcaaaatataaattagtttattctcccaagatgaagactcctcctggggctgaggcagagctgttatcttcagggcaatacctcagattctcctggtgttgatctttcttaggggtggggaaaaaggctgaaagggcatttgcccacaacacatcttaggtaaaaggcacctttactactgaaccaaacaggaggcctagctagagaaagttctagaagcagggaaaagcacagactcttttgtgaggtctgagaaagcaaagaaattccagggtgaaagcgggggactcccctagagctgaagtactctcccatctgtttgttgctcacctacctattctttactttgtattattgggcctgggccaggacttatcctgcaagcactgagatggatgtttgttttctctgggggattagtctttttttttctttttttcttttgttttttgcttttgttttcactgggtcaaacaaacaacactttaacagctcaggattttttcattgtattgacttgtctacctgtaaacttgttaattttttactataataaaattatcatataataaatgaaaaatttcaacacagggcttgtgggcattttatttttctctacaatcccaacagatactctgcctcttaagaaaaaaagaaatcataaggaaaatatgctccttcaaaagtgaatcacaaatatgtttgccaacggaaggcaaatatttttcacctgtctcataggctggactgaaatggatttctaaaactctctaaaaccagaaaagagctgagtgtctccacccaacctccctcctttcacagattaaaaaataaaaaatggagcccaggagacatccagtatcttcccctattggtcacctgggacaaaatctggaacatgcacatgcattgcctggcaggaactcattccagtgattaaactcttcaggaggatgtttcctcttgctatttcattacctatttgtgcagtttgatagctagtaaagtgatcaaaggaactgtggggcatagattcaaaagtccttcaggaagcagaaatagaagaacagtactagaggcagcaggtccctgaccagcaggcccactacctgctgctccagcacacatcctgcacattttcagagggtgggggacagaggggccctgggtggctgttgcattgagaaatctcgccctgctcctgtatgtgcacttgaggccgagagcccttggatgcctggtgacagtggtttcctcctgcccctgccttcctctctggcagactgactggcccttctgctcctcttccccttccaggatgtcctgatatctttttaaaccaaatgccaagtttgccaaaaagtgtctgtttgtgtgtgtgtgtgtgtgtgtgttcaatgcgtgtgtttataccacacttcacaatttgtccaggcttgtattaataccatcaccaggctcaaccctggtgttaattccaagatacttaaatgcccatctaggtgaatttctcaggtaaaccatatattcaagctgtagtttaagctggctgcccgtcatagcactttgaatagactttgtttttgtttttgttttttgagacagagtctcactctgtcggccaggctggagtgcagtggcactatctcggctcactgcaacctccgcctcccgggttcaagcgattctcctgcctcagcctcctaagtagctgggattacaggtgagcgccaccccacccggctaatttttgtatttttagtagatacggggtttcaccatgttggtcagactggtctcgaactcctgacctcatgatacgcctacattggcctcccaaagtgctgggattacaggcgtgagccaccacatccggcccctgaatagacttttactcaaggttcaccatgactttcacatgttttgtattggagtaaaatgtgccagtggtgggctaaagaaaattaactcatttcaaattcaaacctggttttcttaatttttttaaaatcacagtttctgaaactgtgggctcctcatggcacattgagaggaggaggtgaaactctccaagtctgaagctcctgttataaatcttcctctggcaaagattgtgtgatcaggcttgagtacctcacagtcctagagcaggtcaaaggctggctaggaaactcatttgctccctgtacctctcccctcctttcctgcctttgctcgttctcagctcccggtggtagagtaacactggcttctgattggtgcagggtgttcaaccagagaagaaagagccctggaaaagaccgagcccctaacagaggaaacggaggatccagagcacccagaaggaatacacggtaaaaccccgataaagaatacacagcagaggcgaggaaaaggctctaagcactgcagagggccagagcaaaacatctcatggcaagggtggaaagaagcctaggaaactgactctctctgtggacaagtgttaaaccagatcccttctcagaggtccatctgcatgtgtgtggaatgaatggttcagcccagacattagcgcatatttcctggagaaagcaaataccaactatgtagtgtgcctgtgcccttgttaggcaaatcccaagtgagttgcacaaatgtgctgacttccgaggatttagcaagaacaataactttggtcactgggacttaaagcggatatgagctataaggaaagacaaaaataaatgcttctgtgtccagggggaaagagactccaggggagctgactacacttcacttacggcttacaaatctagaaggccattcattgaaaccatcagaagcctttcctgacagtggaagttacctaataatccctaaactgacgacccagatttacaagttttgttttcctggcttttgctgccctcatcttctctcttaaactagttctgtatttctcccaaggcttttcattccctaagcatacgcatttctctgtggccaaaatgctctgggtttagacaggcagcacagcccctgggctctgcctgacagggcaggagagggtctggcctttatccctccagcccaccccaggggccatttcataaaactaaagccagagacctgcagcccctcccagagttagactgcagtacaccatgcctctggcaagatcctcctcccacagtggaaagtctaagccaaatcaggaggctggggactggttccacctcagttgcaggcaaggccaggaggcacggatagaagaaacagtggactttttccccctagggaaagaaatgcttagagctacagtattaagatgacaaattaagctgtgccatatagggtgaaatgaagcagggatagatgggaggtcagggagaagtgagagcactcggtgagggtctgcactggagggggcatgggaggaagaaggaggggagtggggtttgagggatggtgatgaggaagcgtggactgccctacccacctattggaaaacctgggagttctgaggagcaagaagccttagtcaaagtcaactcaaagattcaagccaaggtgactaagagaatggcggtccagaaaaggtcatgggagaatctgaaggcagatgttgttttgggaagatgaagaacctaagccgcttccagaaattcatgaggaaatgccccgtggactgttggcaatgagggcctaggaccaaggttgagcttggggccaactctccctatagacagtgagtgcattctgacaagcatgggctctgggttcaaatcccaactctgccactcatgcctatgtgtccttaataggacgcttgatgtctctgtgtctaaggtttcctggactatggaaatgagcctaataaatgtctaccccttaggaccattgtaagagtacattgaggtaatttgtgtaaagcagtcgaagcagtgcctggcatataggaggtgctgtataaacgtttgatgctagtattactattattattctggagtcttccttgcaacggtgatagccgaagccacaggggcaggtgacgttataggcagaatacaagggcctggagacagagccctggggccatgtaattaggcattatgtttacatcatgttcattttttttcctccaagactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtgagtcttccagcagagaagctggctgccatgctagcctgtcatttcctggcttagtctttccctatcagcggctgtctactctttcccacaaattttagtgacaaatatttgcggccccaaaaatgtgtaaaagctttctgcagtattcaaagatcactaatatgtattctcttgatggggaggtagaatacgtttattgccccttttgtgtgccggggaagtggacattcattcagagagttgaagtgactttcctgaagccaccaagttgtcatggctcagcgggggcaaaagccaggcaccacagttgcctcttgtttctcacaccttgagtctttccccccatctcaacagtccatggtggtgatcaagtcatggccactgtcatcatgtgcatggaagctatagagtcctcctatttcctttctcttttcttttctttttttttttttttttttttgagatagtaaccattacccatgctggagggcagtggtgcgatcttggctcactgcaacctccgcctcccaggatcaagcgattctcccacctcagcctcccaagtaggtgggactacaggtgcataccaccatgcccagctaatttttgtattttttttttttttttttttttttagtacagacagggtttcaccatgttggccaggctggtctcgaactcctgacctcaggtgatctgcccgcctcagcttcccaaagtgctgggattacaggcgtgagcgaccgcaccaggccgagtcctgctattttcaaggaacattccttttcctaccaatcattaggcaggcttcaacatcagctgatgagggttagtggtcgttctggagaaagtgaaaaaagaatcagtctctagaggggcttgtggagtaaccgcctggtaacagaaggtcagggcagggaaggcaaaggggctctgcgcggatctctcagctccgcaggcgccccactctcctccaagggacccgagcgccatctgctgagaggagaacacggcccgccatggtttcccaaggagcagcagacacggacctcgcagggggcagcgaacccacgtgacacagtcttcaagtcctttggagagccccaggaaggaacaacagcgtgtacaccctgtgatggaatgttctctagggcggttcagtgtgaatggaatgtggggccggtgccattctaattggttctgtttccctctagtggttgatcgcggagatttcggcttctccatcaggacaagttcagatagcctgagatggtatcagaactcagggacagagctgggtgtggcggccctgcatccatctgctttctctccatgctaactgatatggtcagagagctggaagcaaattccaggaccccagggctccgcaaaggcaaacacattacttcatcggctgctgacatgcaacttcccccaggggttaaaacaatgtttaatactaacagtaataatatttttgagttttactttatgctggcgctgttctaatgttgtaagtgtattaactcatttaagccttacaacaacctaaggacatgggagtcatagttcccatttaaaaaaaaaaaaaaaaaaagcccaccattgctctgaggctttttatgttttggatccaaagctaatattggtggtggtaattcccatgcctggcttcgatcaattaatcagcaaatgcctaggactgcttagggttctggccttcatcaagaccttacccgggctttatgatgatgacacctggcttttcaatagccatgactgctcacccaggaggcaacgcctcgagtcatgcaccgaacaccttttattgatcctctccaacaccaggctccgtgatggctgagctggggacacctgtgactgcacgtgaacattttgaggctgggaatcccaaaggccctcggcgttggcctgggagcaccatgaaacaagtagaagcagagaaggatggcagaggtggccctctgcattagggcctggatgtatacactggtgctaagggggccccacagctaataggggtttgagtttgactgacagccccaggcaggaatctgtgagagttctcactgaacctggtgtgggggtggccctcctaaggcatgttgctaaaggccatctcttctgccactgacgcctgtgttctgcaggtccatcctgacgggtctgttgccaccaacctctgggactgtgctcgttgggggaagggacattgaaaccagcctggatgcagtccggcagagccttggcatgtgtccacagcacaacatcctgttccaccagtaagcgacacaggaactgagaccgccccatcccctctcctcacctctgcccccagcacacttctctagagcccagctcaggggtgccaggcctgggcacaggcagagatacagactcttatttggtttcccctatgtttaaagtcctttgtcctacttgcagtgagaattgtccctgagaatatgggactctgcctctgctgctcagagctgagggctcctccctcagaagggtgaggctgccttcgctctgacagagcagctgatcgatccccgagccccttgtgcagccctgaagtacttcctctctgggaccaaagacaggagaaccattgttcctttttcctgttgaagccacggcctgaaaggcaaacttttcagggggcttttcagttactttttttccccaataagatatcttttatttcttatctaagaagctacgcatagtcattgtgaaagaaaaaaaaggaagggaggaaggaagggaggaaggaaggaaggaaggaaggaaggaaagaagggagggagggaggggagaaggaagcgagggagggagggaggggagaaggaagggaacaggagggaggaaaagggaaggggaaggaggaaggaaagggaaggagggaggaagtaaatataggtaaacaaaaaattgaaaataaaagtcacctgtaatttcactactcagagataaccgctgagttataacattggtatataattttttagaactttctcctatacatgtatagatagataaacacatatacttcaaaatgataaagaatagtaaaactatgcatacaattttataacctgacttttttttcaaaaaaaaggattgcttttttaaacataagatatcaggaacatctttcatgtcattacatattcttctataaaataatatttaatgtttacagattattccattgtatgcatgaactatgtaagccatcctcttattagatatttaagcagggtctgctatttttgtattgtatcataaacaccaccacagtgagcatcttgattgccaaatcaagaatacttgtcctcaattatttctgtaagatcagctgctggaagtggaagtgctaagccactgcttttctcgttgtcccatcctcctagcctcacggtggctgagcacatgctgttctatgcccagctgaaaggaaagtcccaggaggaggcccagctggagatggaagccatgttggaggacacaggcctccaccacaagcggaatgaagaggctcaggacctatcaggtgctcagagctggatggagacagggccacagatggcaaatccatggctccccagtgcacccaggaggcaggggaggcttggagcaggagagcttctaagggtgggaacacctctgtgaagttacaccaaaaatctaagagcagcccccagatcattttccctgcagagcactgtctcacagcagcctgggttttatttgtcctgagattgatgtgcttgaacagtcttcaaagggtctgatccgaggaggtgagggttgccctttctgcatttacaaagcctgaacagtattagggctttgaacgctataaacatctaagaggcagcaccaaaccactgctgggttaaggtacccccacaatgccacttgccctgggcctttctcttcctcaccctccacagccccttaactctcccgtccttcttgtgcctccaggtggcatgcagagaaagctgtcggttgccattgcctttgtgggagatgccaaggtggtgattctggacgaacccacctctggggtggacccttactcgagacgctcaatctgggatctgctcctgaagtatcgctcaggtaacagctgctgctcagtctcctgggctgggctctcactgcagccctagctgtggtccccactctctcacctgccattttgtagctgagtacaggaaccacaatgactacactcagaagggggtttatcagtgacttggtgaatctaagttccagctaaagcctcctgaggtttttacaaatataaacagagaatcactgatgatgcaacctacttcccaaaatattttagaaaattctcttgacctgcagcccttctgtctggaataatggatgctactctaggtgaatgtcttctctgaccatggggacccaggtcacctgcaaacatacctagaagctccatagctgtcagatgaccactcaggaccagtgtgagggtgacctgctgggcattcagtgctccagagggtggccacagatggaagtggctcctctgtcatggcacctctcagacaaggggctcagatcagaagagacagcaagcagagctgagtgcccatagaggtaacagcacggttcaaccccgtggtcaagccagagctttcccccttgctctactcacacagcgttgccccgtgcctttctctgagggtttgtcatcctgaaatcctcattgctattttctttctttcttttcttttttttttttttttttttttgagacagaatctcgctctgtcgcgcaggctggagtgcagtggcgcaatctccactcactgcaagctccgcctcctgggttcgagccattctcctgcctcagcctcctgagtagctgggactacaggtgcccgccaccacgcctagctaattgtttttgtatttttagtagagacggggtttcaccgtgttagccaggatggtctcgatctcccgacctcaggtgatcctcccgccttgtcctcccaaagtgctgggattacaggcatgagccaccgtgcccggcctgctgttttctgttaatgacatctccagttagtgagagtatgcacgtgtgtgttctttatgaagagtataaatccagagcttaatgatccagaaaatgtacatatgaaactccctagatgctgaccataatacatgagcccctaatatagagatttatttgaatcagatcctatgctggatacagagacactgtgtgtggcaatgctttacagtatgtaggaagctatgaaatgttagttattattgtcctaatatgctggaatttgctgctgaattagttcccttgggtttttttttttagttaactcctgatttttgcaactatatagccaggaaattgctgtacaccctttaccaacaatgcccaacccagggcaggcctggtgattgccctggcccctaccttgcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggtaggtgtctgcccagggaaggaccctggcctgggtgagaaggagcacacagcacggggctgccactccagacatggctactcacacaggctctcgccaccagaatcagtgtctttgttctgggaccatttgcagaagatttcgatgaacacattctgaagcctcctcctacagagatgctttagccaaaatgaaacaactagctttaaatggtctgcaagtattacatgccagattacacaccagtttggtgcggtttggtgcaacatagaagtgagtgtcttattctgtaaggttaggctgttttaagagcaattggttgagcttcatttcaacattaatattccctaattaaacctgaatttcagtggtaagtgaaaactaagaagaggcctccttgggtgctataacataaaaatgatgaaggcaaaaagtaccaaccagcagagaccacttcagcacatcaggagacccagttttatgtctgtgctgcgaagtgaacaaactgtgtcatcctaggcaaattatttaattcctccttttttttagtatttttttcttcttcacatggaacatgaagctaatgacctctgcttctatttcttagggatgtgaagataagtgagataaagtattataaatgtgctctgggcttcttaagaacaggcattgctcacattcaaatggtcatgattatgatatggcagcattatttatgcctctggtttaagtgtctggctgccgctggggtttcctatgtccatccacggggagggaggcacagaatgtctcccacaggcagaacctacagctgccacataattgatgacaagccaaagggacccttggaggttctgctcctctctgtgtgtgactcacacactctctaggataaaatcaagcgactacaccctcaaaatgctcagatgaattaacagattaaacagtgaagaaaaaaatgtgttgactacacttggcagtgagaaataaataaagcgggcggtgacagcagctggcatcagggagaggctgtcatggaagggatgtgcatcttgtcagtcatcccatccatctgttgcaggggacctgcagctgctcgtctaagggtttctccaccacgtgtccagcccacgtcgatgacctaactccagaacaagtcctggatggtaaggactggacgggccatacttgggttccgtctggcagccatctcccagtattgctgggtgtgtcctgttgtgatgcattttaatgggagcaaagagaacactgggtacttctgcaggtcacacagttgttcttttgctttgagcttctttctcctcttccttcttccttcattcccaaagggattttaaaagtcatgcacctaaaggccctctccctttaatgaggaatacactctgtgctcttacccttagtaagccatcattcctggggtccccctgccctggctccaggccacattccttagtgtctggggagagcttcttctacatgtgtgccgtggcgccctctagtggaagcatggtgatgcacggctcttccagtgaattcgtggagtcagagattgcacatgtggatggcaagtctggaaatagcatacacccctgttatactcctgattctcccctcagcttcccaatttcccagtgattctccctttaattaggatgcactgaagctctcaggggtgcccccatctccaaggagctgcagtggagaggctatcccctctctatgtgagagaatgtgtgagaagcgtattcccacacaggagcaaaactaaacttacgtactgatgcaggttaatgaatggggaaagtatctgcttatcaaagaaaaggcatatttttctatttagcacaaactttttcaaatgttaagaatttactaactgaaatctggtgaagcaagagaaccgggcaatatttgcgttgtctgatcattacaactggagggaacatgctcagagaggcatcatcactgttcatgcacctgccctctctttacactgagagaccctgtgatgaacagaaaacatctttttaggatgacatctctgggtctttctcctagcctgccttgctgtgggtacctatctccctgctctctgaaccttggtcaagaagtttatatttgttttaaattgatactaatatgttaagttactgtgatttgccaaaatcagattggaaacagggcctgcatggctgaatgattcttttttttaaattactttatttctaaataaaggttttctttgtatagaatcgggatgctgtgaatggtgggaaatgcactaaatagttatgccccaaataagaaagggaaaatcatttgaatccccagttagctccttgaaagtcttttcacttaaacacacccacataccacacacacactcacagacctccctcccagatgcccaaagccctgctgacctacagagctacttctggaaaggctgacacatgcctaagacacaattcctgggaatccagcagctttgggttcaatttccttcctaaaagaacaatgaatatgacccctggagagctattagggcagagctgcttccttaacgtaaaggactctccagcctccgtatgaagtcatctcagagctaaagacaatcaagtccaacttgcagatttgacataaagcaagacttccaatccggctaggcagaaggattttggttgaaaaccatgaaatcccttcatatggatcattttttaaacaacaaaaaaagaaaagaacctactgggtgtccacaactctgagagctgctttctgaagagtcatgttttgagtcctggaatccctctccctttgacctgcctctcaagacaatgtgcgagagaactctctcttcaagtgcatgcaagtgaggttttcacagttagatttttaattttaaagtaatacacatttgtacataaaattcaattctgactgtatacatgtgtcagataaacagttgatacctgacacttgttcacagtctatgatacgcaccgcatatcctaccctctcccccagcctctctccatggcttctcaaccccccctctgcatttcctgtgacctgaggattcagttttgtttgtggaggcaggtgcaatcccaagagaaactgtgcaatcttctgagaagttagagtaggcatgtgtgtgtgatttagggaaggtacttctcactcagcttggtcaccggttccaggtttgtgtcttgggcaagtcccccatagctggtgacagaccagaaaaatgaaaacaactttgacttagccctcaagttttcagtgaatgagaatgaaaaacaaccatgagtaagagatttcttaccgagatgatgtaaaggataataatagcagccagcactcacctatgtgccaggtatttctctaactgctttgtgtagtttgactcatccagtcctcaaaaacaacaatgaagtggataccagtattttccccttttcacagatgaggaaagtctaatgtgacccacccaacataacatagtttgaggggacagagcatttcgttgaacagaggaggaactggcacaggaaagttgcatgacccccccaccaacctccgcccccaggttgcacagctagctagtcgggaggactttgcttccgtttccctctgcctctcaatgatgatctcagggccaactaagctaaaagcagacttgatggagcatcagtcctctgaaagagtcactgccgagatacaaaatacctcttcttcaaaggggaagtggagagaagtaggaaatctgggtaacctcacagtcttccagtttctggaaaacagagctggcatcagtcttttttcttgtcctaggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagaggtaaagtagagattccagctggtttctgtcaagtgccagaagtggcggttctttgaaaaagtctaacattagagcaaagttttgtaaaagcaaaaagccatcgttccccacccaagcatagcaactatctttatttttggcatagttcccccatctctgcatgcatacaaattttatgtacttgtggttactgtgtgcttacgtttttgtatttatagaagatgatgttctcagatagagtcgtaatggattttcttcccattatgaagcaatacccaacaaaacagagcttgggttagatttttctgagaataagaatgactaaacaaaattctctctttttttcttcttgacagatttttctgaaggtcacggaggattctgattcaggacctctgtttgcgggtatggtgctggagccagtggcttgttcccttccttgcctccctcccaagttccatctcgaaagtctaaggggctgggcacagtggctcatgcctgtaatcccagcaatttgggaggccaaggcagatggaccacctgagttcgagaccagcctggccaacatggtgaaaccccatctgtactaaaaatacaaaaattagctaggtgtggtggcgcgcacctgtaattccagctactcgggaggctgaggcaggagaatcacttgaacctgggaggcagaggttgcagtgagcagagattgtgccactgcactgcagcctgagcgacaagagcaaaatccatctcaaaaaaaaaaaaaagtctaaggaaaaagtcatgaaacaacaaagcaggcaaatactcctccatagtatctgactccccagtagtaggcattttgcatcctagatggctttgagtgacaaaggaataacagactgagttaggtctagatggggacactttggatgaatgaggattcttacggaggtcaggttggtagcttcatccctcagctcctcatgctgtatccccagtctctcggcctgccatgtcatcatcctcatctcctcctgtcatctccaccaggcctctgatccatctctgtctgcatgagtgacagctggcagagtccttaatgtttatcaaatacaactcagacgtcagtctcctggcccctttgagatcaacataaaatcattttgaacccttatttagtggtctatgggctttgaaaacatggggaccaaaattcctgtggattctagaagtctctcttctacatgtgtcagcctgggcaccaactagctccttccatgaacttttatcaaacccacagccacacaaagcatgtgtgagtgtagcagagtttacagcagagggtggagggtggggagatagatgtgtggaagggttacctgccacacaaacagaaaccacttctgatagaacacgaggtgtccacccacactgtaaaatcctctcctggtacaggcaaagctttgcagcgattctcctttgctgcccctgggctcctaacacctcctaaaccaccagttacctccttctttccagtgtggcatatttcagtgttttcctgttggagtgtttcctttctatgtggattctggaatcagctcttaagataacttggttttcatctttcttcataatgatcccaaacatctatctactatgcctagaactaccaatggacacatataccagcccagatatgcttcagcccatcccagtacatcgcatggtgaccaaaagatgtagtcgtcctggcacagtgggtgtggggcaggaagcagtcctctccaggggacagcagcaattcaccacagaacccaagtttctttcaagctctgctgacacagaaattgaataatctcagctcacccaatgtcaaagactcatattaaccaagaccagaatgaaaatatgctaatttatatcagaagctttgctggattcaagagttagggccttttacctgtgcagaatattccttcttgataaataggccctctcaggagaataaattacacatcagaggactgtttagtcagcataggcatagaacaggatgttccaaagatacagtcaaggggagtgggtaagagtgtagcctctggagtgaggccgaccaaatatcaaacctgagcttcataatttgcaaactaactggctttgggtaagtacatagcctctttgtacctgtttccccatctgcaaaatggagataataatagcatctacctgtagcattgttgagagaattaagtgagttaatgcttgccgacttataacacagtatacgatcactgattaagacttagcaactctaaactaaatgtttacaaaccatctcttacctcaaagcacttaacatccattgtcttatttgattatcactgtaatcttatgaagcaggcagggcaggggtctgccccatctggggggaactgagctcacagaggttggagggtttgcctaaagtcacccaggccactgggtctcactctctggtcttagctctgtaatctaggatgctcaatgccacactctcagccacttttcagatggctaagtacatttgttttgagttagctcagtctcagaggatgacattttctgatcttgtctccagtgtttaaatgaacctgtagctgtgcattggggtcacacaatgcgtggcatggagagggtctgtggctgactgccacggttactacgtgaaaccatcattacagcagttactactgttactgcctgagaacatcattacaagactgaacgaagggatcaacatggaaatgataacaaaaaaaccaaagtaactgttttaaggaaaggctagcatcgggaagaagaagagagaagaagagaagaagaaaagggctccctgcttctaatgagtaaaggcagctccctaagcttctgcagcccttcattatttattgggtaacaggaggaaggagcaggaggtaatgattgggtcagctgcttaaatgatcacgggttcatgttgttactgacagatttcaattatgcctaatcataagaaacatttgtgcagcctccaacaagggtcaatgccacttctgaaggggtgactcatagtcagtaactagaaagcagcagatagctagggacaaactggcgattctgaataggcctggaacccttagctctggccaggtcagtgggctccagtcaggatggagccttcagggagagatcaaagctcagaggtttgagatgatatcagccagcaaagaggaggggcagtagggatcctcccagagggagggccagccatagaagacatcaaatctgagcccggatcaggagaaggagcctgcagaactggggctctggcaccgagaacctgcagaacttcgcccctctgagtgcaggtgccagggctggggctgccacccagccttcgcatcccaggcctggcacgtcataggtaaatgtagttgaaaggatgactgagctgatccaattccctttacaactgtccttgtcctgggggacttgaggagggttaagaaagcagctggggaccaaccaacagtcctctaggctctccatgtccagcaatagttgttcagcaaatgagcattaatcagtgactataaactgtagcttcaacataaccgacaacttgcaatggtttctagagcatgctcccatgtgttatctcatttaaatttccaaaccaatcctgtgaaatgttctttttttttttcttttttttttttttgagatagagttttgctctgtcacccaggctggaatacagcggctcgatcatagctcactgcagccttgacctcctgggcccaaggggtcctcccacctcagcctcccaagtagctgggactacaggcacacgccaccgtgcctggctaatttcttttctagttgtttgtagagacagggtctccctatgttgtacaggctgatctgaaactcctggggtcaatcaatcctcctggcttggcctcccaaagtgctgggattacaggcatgagccaccatgccttcattttacagataagaagtctgagaaaactcagatttaggcagattgagtcacttccccaaatttatgtatcttgtaagaatccatattcaaacctcagtcccctaactcttagttcattactttttctaccacttctcagtatcctctaagaattcagaaagaaccacatcgactctgatttttcatttgtttaagtacacaggtaataggtgaatgtattttgttgtttaaaaattcatataatacacaaaaggctaaagtctcgcttcccacttcctctcccctttctacccaactctgcctccccagggagagcttctgctgacagtcggtggacattctttcagagttttacaattatgtgtgtgtgtgtacataagatgtcagtttttctttgtgtaggatacatgaacatgaattttaaacataaatgtgagtgtattacacatattgaccagcaccttagtttttttgtttgtttgtttggttttctttgtgctgtttgagaaggagtcttgctctgtcacccaggctggagtgcagtcttgcaatctcggcttacgcaacctccacctcctgggttcaagtgattctcctgcctcagcctcccgagtagttgggattacaggtgcctgccaccatgcctggctaatttttgtatttttgtagagagggggtttcactatgtaggtcaagctggtctcaaactgctgacctcaaatgatccatccacctcagcctcccaaagtgctgagatgacaggcgtgagcctccgtgcccagccagttttgtttttttattaaccaagttacgtattttaaacttctccatgtcaatgcttttagagctattttgttctctttaatgttaatagagaattttaaggcaatttcaggtgaatctatacaatttctctgtataagtaatttacactagaaatagatttttataaagatgattaagctaccagcctggtatttcattgctgacttaaatgaagaggaaaatcaatgctgtaagggaaaaaaaaaatggcattagagatccagaccttataggcattttccaaattattaattcaatctctcaaaacaggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcaggtactattgtcggtcggtgtttagctgagctcagtggctcctctcccagccttcccctcctctcctgagtgttccttcaggcatgggttataactcagcaaggagcaccctctttagattctgctggttttgtttcctgctttccaaacccttatcttgattcttggtaacatgaatcttctttgtaagttggacctcccctagcaaagaaaatagaataatagtgaaaatgttaatattgtttttatttttacagtgagggataaagtcatgttttcattcatttttgcagtgaccctacatatcaaaatcattgccctcttttttcttttaatgttgtttaatttagaaaaagaagctctggtttaaagaacagtgagtcacgtgacttgctctttgaaatgccctttgaagtctggctgaacactgggctgcattcagattcttcagtggccaccagaacattctgttttcttctgcacatcttacctttgcacaccctgcttattatgttcccccagaagcccaaccctctccaccaggggctgattaggaggctgcaggataaatgtttaaaagaatgaagatgtgtgtgcacgcgcacgtgtgacatctccatgccacagtcatgtttattccacgtctattctcccacagatcgtgctcccggctacctttgtgtttttggctctgatgctttctattgttatccctccttttggcgaataccccgctttgacccttcacccctggatatatgggcagcagtacaccttcttcaggtgcgcggactcggggtcaccattctcctctgtgggtttggggcacctgggtcacatgctgcttagaagggccctgaccttcccacttcactgggaccttcaccaatgagagaggggaggggtctttgggctgcctgcagaaaggaacttaatgtatctgccactgcttggaaaggcgatcctagtggacaggcaggactgcttgggaaggccgaatggggaaaggaatgcaaagcttaggtgaatgggttgaagcgccatctttttgaggcataggtgacatgccatcagaccactgcgagtgttcaggcagcctaccgcactcccaggagagctagcgccatcccaaggcagcattcggtgcctccaatacatacctggcacacagcagctatccagtaaaggctctgagttgcatgatgttggcacgcgcctgctctgtcccagtcacatgtctcactctgtctagcatggatgaaccaggcagtgagcagttcacggtacttgcagacgtcctcctgaataagccaggctttggcaaccgctgcctgaaggaagggtggcttccgtaagtgcctacgcgcccctgtcctaagaagactagctcccctgggaggacccaacggtgggttcaagatggcaggcgttggggaggccccactcaatcctgctctgctggtcacttccatgtctctgaccagcactcccccaacctctccttccacacttgtgtgcagggacattcactacctcctaggaagcccccacaccactggacagctctatatttctcagcatagaagttctatgttgagttgacagatgattccccataacttatttgaaaggcctctgagcagggagggagggaaatagggttatgctattgtgtgattgggccttgaatggcgtgagtgacacagtggccagtactttgtgatagttgtgagtctggagaagggagttagcgaaggccattgacatccaccaggaatcctaaaagttcaatataattttaacttttctccctcagtctttttcaaagctgtcaataaggaccaaaacagactaatttcaaattcctcttctggttgctgtgtctctcaacagctagagctgctaggaataaaaagggagacaaaacgatccacaagctagagatggttattccccagccccacacctagtcagtcacaaaaccctagttttgatattgcttgagcagaaaccagcctccaagagaataagaagaaagggcctgggtctaaagaggaggaggaaagggttgggcacaatttcttatgcctagggatttgtcagcaactttgaggctgattatggaatattttcttgtcttccatgagggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccaggtacctgacctccaaacaacggggccccaggtctgcctgccacagagggactaggggagtccctggtatctcctgagtctctcacaaactaacatttcaaactggcagttgagtaggggactaaaccaaactccctgcaccctctgggaggggctccccacagggcgctgtggctgccaactggaggaagccactcaccaaaagcttcattttccaccagatacttcctatttgatctagtagaaaaaatgtgtttaagcactaaaaaaaattaagtcatatgtgctcattatagaaaaattagaaaacacaggtaagtcagaaggaaaaaaaatcatcgcttggatataaacacagataatgtttggtttgcagccacccaaacagattatattccaaatattgtcttaaaatctgatttactgcataatttactaggaacatgcatccatgtcaataaatagacatctgcatcacttttaatatctgtatattatcccattgtttgaatttcttttttttttttttttttttttttgagacagagtctctctctgtcacccaggttggagtgcagcggtgtgatctcggctcactgcaacctctgcctcccaggttcaattcttgtgcctcagcccccccgagtagtggggattacaggcatgcaccatcatgcccgcctaatttttttggtagttttagtacagatggggttttaccatgttggccaggctggtgttgaactcctggcctcaagtgatctacccacttctgcctaccagagtgctaggattacaagcgtcagccactgctcctggcctaaagttactttaaattaactgatctcccattattcgccacttaggttttttagttttcaccattataagcaatgctatgatgtacattcaaatggaaatgtgtttacacacttattaacagtcttaattaagaagctctccatgtgctgtgtctctaacatctgcaggtatgtacacaaatacatgcacagccagcatccatcttttgcagggacattaatgatcttggctctgagcagcaccctgtcctgggagttctaaagtccagaacagattacagtgagcatctcctgggggatttagagacatcaaagaaggctgtgtccgtggttgataatgggcctcccagctgacttgccagggctgggccttagacagccctgtccaatgatttgtcaatgaataaactgttcccaaacaggctatgcagttcagtgggaaagcacaggtatgggacacggagagccccaggtggactacttgacctctctgagccttaattttatcacctgtgaattgggaataactgcttatttcataatattattatgaggatttaatgaaatcatgtgggcaaggaattatttagaattagattcaactcaagtgatgacaaccccaaactaacagcagataaaacaagacacaacttgtttctcactcatctaaaagtctacgtgggtggtgcacgatgttctattctctttctcctccacactaaacaggcctcagcctcatcagccaataaggcaggagctgccttccaggcagcggaatggaagaaggatgaagcaaaacagagggcagagtgtgcacatgtgctatgtttagggaaggttttctgaagttcccacatagtacttccacttacaaacccaacaaaaaaggctatggctaaggcagcagggaggagcaaataatgggagcaactagattttgccacagcacctatcacagtctggtttataaatggttctaggccaagaacacccgatccctgctcttttttatattctaaagcatgtatctttatatttctcaagcaatattttctctctttgaatcacagctcatctgctgcatcatagggatcccaaaagaaggacccaaggaacttgtctcagtcctctgtgccccaagaggaagctttgcttgtttgctttgctgtcaatgctgagggctcctgtggctgcctccactcaaaaccctccagcatcaggacgtcaaggctgtgatactgtaccctgagctcttggccagggcgagggaggggaggccaagcctacctacatggtgtttcatttcctaaacgaacccttacttccacgcggtctgtccagcttagaaacttattttcagtagtgttggtccttggtccctggacaaaatgtaacagccaaagtcctagaaaaaggcaagccagttcctgccattttctttcacttctgcatttcctcactattatacgtgccttccattggagcaaaactgaatgccacgcatatgcacaggagctgtgcgcgctctgtctctctcactcactctttttctctctctctctttctctctcaatctctctgtctctatctatctcttactctttatctctcactctctcactctttctcactctttctctcaatctctttctcattctctctctatctttctctctctctctctttctcacacacacacactcacaaacccacactcttattcacatctgctcaccctagccactcaaacacaatccctcattcagcctggaataagtccagagggcgtgggcctgattcagagacaatcagttgttctcatctgggaaatggggcaatgtggtcatctctagggaccctccctgctctaacattctttgaatgtggtgggtcctgaggtggaagcactctgtccctgacttctagtatatgtggagatagggttacacaaatattttattgggcagaacttttataaaacaatttatcataagctatcgcagccagcagcaatttttccaacctggattccaccaggggagcttggccggtgtctgagtgccactttcagcttgagaagcaggtgactcagtgaaaagagcaaggaggagacagaggcagattcagttcctaggccctgggccacccacctgcaagtttgcagcccagtcagtgcaagtcagctaactgttctgaacctcagtttctctgtctgtaaattaagctaaaaattcttctttcaaagagtgtcaggatgaagtgagatcgtgtatgtagggcatttaacatagtgcccgacacacagggagcattcggtaggtgccagctctcctcctggcaggagagagagaaacaaggtgaaaagagtgaattaaagaagaggaaagtcaaatgggaaaacagggggaggagatagaaagtgtatgaaaaggaaagaatggtgcgcaataacggcggtgtaatgccaccaaaatcccctcaactacttctgggcagcacccttgacagagtgaatgcttttatgagaatgtaagcggaatgtgttcccagatttgcagtaatattgccacctggtggacaaacccatgcacctttgaattttccaaaatatttcgatgaactagcttccagtcctagatgtattttgaaagtgatttgtaaattgtaaggaactattcaaattctttcattaatgtcacaaatcaactgtgtcatctgtatgccacccactattctgggtgctggggacacaacagctcacaaatcaggcaaagtccctgctctcaccaaaatgatatcctacgggggattacagatacaaatacgtaaacagatccatcgggaggaaactctcagatggaaatgagagctatgaagataacacaacagtacatgacaatacagagtgactggaaccaggaacatttctccgaggaataaaatttgaagcgagccatgagagggtctacaggtagagttcccaggcagagtgaacagccaagcacaaagctgcaccaggagagagaggtgctcgccgagagacagggaggggagtgtggcaggtgagctcagagaggggcagggccacacacatcggccacatgggccttggtagtgagtcgagatttgatcccagggtttattggagtggataagtaagcaaggtgactgaggtgctcgggtttacatttttatagttcaagctggctgctgggtggaaaacggaagttggcagaccaaggacagaatcaggcagacccatgtggaagtttctctagtggtctaggtggtggcttgggtagcgtggcagtattggagctggagaaacgcagatggattggagatttgttttggagtgacgccattctgtcttgtcaatggattggcgaaaaaagaggcatcaaagatgagttacacatcattgaagtgagaactagggagatgccagtactttatttagtattttctcagcagctcaatccataaataatttttggaagacaacaagcagtttcacaaactacttataagtcctcaagttccaaggtaattaacgtgggtgtctcattgcctcagagaacacagcgcagcacggaaattctacaagacctgacggacaggaacatctccgacttcttggtaaaaacgtatcctgctcttataagaagcaggtaagaagaaatccttttatgctttttatcctggctccctgtagaagatattaactagggacagaagataattttctctctcaatttatgtatgatcagggcagtagatttttttcttttttatctgatttgagggccccattcaacataaaaagcaattgaggcacatacaagtaaaatgtaacttaagattaattctttttttgttgtttgtttgtttgtttttacatttagggcaagcagtcttaaattttaacccacgtattattaaaagttatatcagaagaccatagaagttattcaaaaatgcagccacatattttaactagttaaaagagagagtaaaaatttggagggaggtggaggagtataggggaaaaggtagaagaaaaagagaaaataagtaagtggcaaaaaagagaaaggaaaaagatagggtgggaaagaggcagcgggacagtgtctgagtccagcacacgccagggcgagccaggtcaactgcagctgtcatattctaactgtgaattatcatctttgatcactgccctttgagatgccaatgaacttttcaagaaatatctagttctcttggctctccagctgttcttatcagccccatccaggatggaacagctttggcagcccgtatcagaacaagcagcttgacaggggcatgccatgccaggagagaggatcctaaggaagcgtggtccagtccgcacaggctctggggctttaagataaaacctcctgtctaactttagtaggactttctgttgcttcacctgccagagccctgaacgagggataaattgacttaattaactagaacacactgcaaatggtgaaagcatttagcaaaacaaagaatgccatccaagccccaaaataaaagcagaataaatagaatgcaataaacagcaaccatcccaaactgagttctcagcagcaaatctccagtatgaaattttggattttgtgcgtgtgtgcttaaaggtggatgacaatgacagttcatgggattgagctctggggtccagagttggcatctgttcatttcccattttgtcattttacccttgattgactgaatgtcagtgccttaactttgggctgtggagtgagtcggaactcccccgaggtgtgcaggtggttgttagagtctcatttttgcagggtggaagacaggagggctgcagccttcattccacactgacatggtcattgccgtgtgttctgggtccagatcaggcatattgacctgacatatgacctgacaacaggaccactcagaaagtccagcatgcgggatatgatttggagagccagtgggggaaatcataggtcctttctctgcatgtgtattcaggcaatgtcccagggctgggcggcttccgcattgcttggatatcggaaaatgcaaaaatgcccctgaagactgagacttcagtcttcaaaatgaatgtttgggaaagaaagttaacggcactgctgtacttgtggtattcattgcattattttattttggctttcagcttaaagagcaaattctgggtcaatgaacagaggtaagaaactatttttatcagaattaaaatctcagattgattcattgttgaaataattgcacacttttaaaaggcacacctcacagccatgaggaggggctgttctgtaggtgctcaggaagtcacaagacacgtcctgaagaatatgtggctagggacatcccagactcagaagacactcagtggtgcctcttcttggaggacataagtgggggtggcattccctgatgtggcgtttcagagcattctcacccaaaaaaagcttctaaaacctccaagtatataacagtttataatactccaacaagagggccttgtagcctaaacccgggacactccttggcccattccttttaagcttcagggagtgtgggccagccccagactcaccccattcctgaggcatcctggaggttgaaatatttccagaggtttagaacctcaccaagtgggactctaggagcctgctgcctcccagcctccctcaggaactgcacctccagaacaggtgcggggctgacatgtatgtgctttcctgggcagattctagaccgtacacatgaaatctggctttcaggattgctctccagagggacctgtggggcctcggctgagacagagagtaggagtgaggcagtgattcaaggccctgagaaagagctcctcctctgcttggtataaccagctaattcattctgttctgttgactttggcttctgccctgcctttgaagggtttgaggccagggagtgatgcactcagactggtgtttccacacagtcacttcagacttccagggcagtacaggagatagatcccagggccagtgaagaagcagagcacaagtccaggcaggagaggctaagggcctccctgaacaggtgtgaggcacagaagccccgagaggtagggatgacaggatgaagatgggtcctgtgctgctagaagtacctgcaaagcacagaggtggcacagaaaaggagtccttggctgggatgggaggagatgacatgtgacatgtgaaagaggacctggagttggctcgatgctcccaaaagggaaaggtgccgaggggagctagcagccatgcaaaggcagagacatgcaggcagtctgggccatgaggagctctggaagtgactcgatatgtccagaataggccactccagggaagggctgaggaaggatgaagttggagaggggcacagaccagatgcagaagggcctcagaggccaggatgagggtttggactccttcctggaggcagcagcagtgggaaaagagttaaaagctggtttgtaaagtggagccatgttgctcgctggtccaggcaattcccccgaaagttcatgtttccctacaaaacccgagagagctactagtaggcgtgaagttcgtggccctggtctgaggatttcctgtttccttgtcaggtatggaggaatttccattggaggaaagctcccagtcgtccccatcacgggggaagcacttgttgggtttttaagcgaccttggccggatcatgaatgtgagcggggtatgtaaacagactggagatttgagtaggatttttgacttgcttaactaccatgaatgagaaactctcatgagtgataacaggaaaaaaaaattaaaaccgtcttgtttgtttgtttacatggtttttagggccctatcactagagaggcctctaaagaaatacctgatttccttaaacatctagaaactgaagacaacattaaggtacttgacctatgtataatctgctctggagctaaaaatttacctgagctggttattttatttttactttcctaccttcattaaattccatccctcctcctgctgaaatctagcaaggaatgtcttccagctaccaaacccttcctgcttctcaaatttcctttccttcactgatttctgctttaactagctgttagtgcagcgtctcagatgtcctctccaccctctaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgtaagccaccacagccccagcctcaccactttcttgtcaccttctccactctttgaacatcctgagaggattctcaccaccgcgaagtgctgatttggatggtaatgctgtttagtcaggcacatatgaacatccgactttcaaataagtgcctcacacttcacataccagacctcttggtcattctttctccccaacatttatgtggcaagtaagtttacatttggttccattcccttttggcttttgatagcaagttgctcctggagcttatacaattattatctttgctatgtgcaaagcagctgccaggaactggcaaagttcagtaaacctttcagctccctcggagtaattatcttagattccaggaatttcctcagaagagcatactttggagatgtcgacagagctttgctaccctcaagctgaggctcttcttgcacagtttcagccagtggagacagtggccttgtgcgttttgtagtatgttcactctatttgaggcctacatggaggaggggttggtaggagcacctttgttagtgcaaacttcagcaacgttgtggggtcctgattttactatcctagcacacgctgagtgccagtgaacatgcccagggtcatccactaaaacctgggccttggctccttggtgtcttcctctggacaccctagggccctagactgtcctctgttaattctcactcagccacactttcgtgtgtctccttccagtcatttgttctaagcttactacgtgtatggatgatatgatctgtagttttatcaaggtagtgactaccacataggatacctttgtggaaattagtaaaaatgctcttttctgcaggtggacactgtcccatgccaggggttatggcttgtacataaagttcaggctggctttagccccaacttacccctcagccagatgccttctatttgtccgaggaaagaataaatagagccaagtccctgtacaacttgcctgccctcttttcacttaaatttacatcatgaacatttccttgtgttacgatgtacttcttgaaaatgtgatttaacaagatgattattaacaaaagataaatctcacagaccgtatgtctgtcaacatagaaaattcaagagactctatagacagattattagagctaatgagagcattgcagtacataagattaatataaacatctatttctatacaccataaaaataattagagaatataataaaaagaaaggttgtctagaaatattcacatgaaatagaaaggcaacccgcaaatacccatttaaccttggtccatatggattaagacagtttagtggagtgacagcttcaaggtagagaagaggaacctggaggccacacctgggcgggtgtaaggccttcccaaagcctgactttgtatcttctcctccttctgctcttccctcttcatcgccctctccctgtgtctctggccctgctgcaggctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcgtaagtgtcagtttacagcgcctccctcccctccgtgggcccaaggtggagcttgtgtgtgctctgaaggaccagaccaagaggggaggggttctcacggtgccagggctgctgaaaggcactgggccaagggccttgtgtatctgctgtcccttgacatcttctcagaaaggcacagaactaggagcccgaagctaggaaaggctgtggggtgcagcttaacaactggtgaacgggggctctctatgtcctgcactgaggggtcttctgacccatcaaataatcactgcaccgcaggcatgagtctggccttcctggcatcagtctggcgctgagaaggtaatatgaaggggtctttcaccccaagtccccttctcaaatcctgccccaccttcaaaagggtaaaggtaaaactttccctgtggtagggtcaccagataaatacaggacacccagttaaatttaatttcagatgatgaataatttttagtataagcatatgctacttcaaatattgcacaggacatatctacactaaaaaaaaaaaaaaaaaaaaaaaaaacctggttgtttatctgaaactcaaatttcactaggcatcctagatttttatttgccaaatctggcaaccccagccagtggccaaaataataagaccttcacttattagattaaccaccgctacagggaaaaatgaagaaaaaatatttattaaatcaatagcacactaccaccttcctgacaaccaaggttggtgggggtagggaggggtcaggatagcgtaccctattacaggctgcagggtcaaaggaattggtagtaaaggcctagttataatgtaacagggatcattatgacatcaaccccaatttattctaggtgtcttgagtagtaaaatctcaacattttaagaccaacatgagcctccatttcatgtgatgataagatataccaactgatggagaccaacacaaatgaccttctcatccatggttttttaaaatgatggtgaatattggaattcctgaagatatgatttctatcttactcagcttagtaagcagctatcacttaacaatacaaaaccagagattatcagtagcaactaaattatttcctctctcttctgtctacacgaggaaacactcataaatgcacggggaggaggtcagaacctgaaagcctttctttggataagagcatcaactgcaggtaccacattggccctgtgatgctaatataaaaggagctaggcccaccggtaccgaaaagttacttagaaaagtgcggaggcttttaattttactttttttaaaagataagaaatagaatttacacacttggggctggcccacgtgtttctgtgtgtgtgtatgtgtgcacgcacgcgcgtgtgcgcttacagggatctctgagcctatggagagagatgtagctaggatagagtggacatctgaggtgggaggtgatactagctggcagtccaatgaaggggtagaagatggtaggcatcatgttagcaggctttctgatgctccagaattttaaagctggcctggaatctcacctccgcgatccatcattttggaacttaggaccaccattagccagtggcaaaaaaaaagttgaatgaaggaacaaacaattattgcttatgtaattcacttagcacatatatgatgttttaaattcttatatgtgtcatctatttttctttactttaaaattttgcaacagttacagacttatggaaaagtcacaagtacagttgaaacctttttttcttagtcatttgaaagtaacttctcagcaagatgccccttctcatttatttctctcttcctgtctctctctctcacacccctcagcacgtccgatgtatacttcctacaaacgaggatacaccccatacaaccacaacacaaactgtcaacatgaggaaaccagcactgatgtgtcatcaccacctaatcctcacaccccactcctctttcgcccattgccccagtgatgtctttcagaaaaaaggatctagctcagaatcatgcatgacatttgattgtgctgtttctttagtctcgttcagcctggaagagttccacagtcttttgttaacactcatggtcttgacactttgaggactgcaggctggttattttgcagaatgtcccttggtctgagcttgtctgaggtttcctcttgcccaggttgagggtgtgcatcttggcagcagtatcagcaaacagatgctgtgttctcactgcatcctatcaggtggcttctgatttcaatttgctctgttactgatgatgttcaattcggtcacttaagaaggtgtctgctgagcttcttcactgtaaaattactcttttcccctttataataaatacaaatttcaggtagaggcacttcaaagatatataaatatcctattcattatacaattttccatttattcatccatttatttatctctgtatgcagtcatggttcatgtgttaatcaatggactatgatccaagactatcattatttattttgatattcacattatccccactgtggtcagtggggggccgttgaagctggcttctgtatcgtcttgacttgggtcctcatgcccctggacctcctccatgctcaatggcacagcaagatattccaggctcatccttccattatccccattcctaccctctccccaagaagccctggttcctgccagtgggaagtggccctcagaagccaaggtctgagtgctagatatgttcattgcctctggagcaccattggtcccaggccttctcagtgatagaactagggaagatatggatgtacacacacaggtatgcacacacctctatctatagttctctatctacctatacagtgaacactatgagctctccaaaaccaactccacagggctcattctagttttttttctttccacatctgtaactcccttctccaacagtgagacgctggcttctctcactcccaactcatttatctaccggacctatacacctgaacagtgcccaactctgccaccatcccctccccatgtggatgccgtcctctccctgctccagctgcctctgctgcatgcaggtcctcctcgttctgctctggctctgataccctgcaccagatcagcctcctgtaaggatatctttctcatcccgttgaggcctccacaccccacggcaggttgccccctgaggaagcccgtctctggttcttgccctgctcctgatcaccatggctcctcccctaaccccactgttgccgtcccctttctgtgcccagtatagtggctgtaggactaaattgtttaaaaagggtatcattatttatttgagctttgtgaagccaagaactaggctttaagtttttctgaattctgaagacatgcttagaaagaagaatcaacaaaactttatgaccaaatagaaagagtgagagaccaggcagaattttgtaattgatcctttcaaaagatacaaactaaaggttcccttggcagggaggtagggcatggggtggggtaggaggactagtgacagcttaacatatgtttgccaaccaagaactgtttaaaaagcaagtcgaatcagaatcccagaccctacgagctggaggagcctggccccacccctcattttgcagagctggcagcaggtctgagaggttaagtgacttgctctcctcttctctttccgagatgaattattccgtgagtgctgggctggtggtgggcatcttcatcgggtttcagaagaaagcctacacttctccagaaaaccttcctgcccttgtggcactgctcctgctgtatgggtaagccgtttgggccattagctaatgcctctgaagagaagcctggtggtgggggtgggggatcatctcctgacagaaaacctgggctgtcctgtggtggtagcacccacaagtttagcttccggccccaggtagggtctgaagctgataaccagggatctgtctggcttctgattctgactccactgacagaggtatctctgaggcctggtcctgtcagtgacaatgagagaagtcccacatgatctgaatctcctactcaaactgaggccttgaccaaagcctgggggcagccattccccaacccctcacccagctctgactctcactcatctgtggccaatctgtccacctcagtgtccccatgtgaactggccaagagttaccgcccacagtagaagactccggccaaaaagctcctcctgagtcagggacagaggatgacacaggggttacatcagcagagttacagggcccagcatgcaactttctttcccacgtgtgtaaatttgaatgagtaattcatccatctcggcctcagtttcctcatctgtaaaagaaaatagtgatcctggtccttcctctgtgggccagtagagccttgccaaagcattgttctccacatctttctcttggaaatagagaatttgggaaccaacctgactataagctgtgaagatgagctcactgggctcatctgagatgacctcagctgggctttgctgacccaggctagagtgggaggtgttgcaggctggagaaccctcctatgaattgtacagggctttgtagtttacagagtatatacacagctagcagcccatttgctcctcacaaaaccccatgaagtggtcaaggcaggcatcattatctccatttaaagttgaggcacagagaccaacaaatggagtatctctctggtcccctgggactctggccagttcacacacatcacctcaggtgtaaggggagtgcattatatccagacgtattgtaggtggaatggaatgtggaactccatcactctgagttgtctcatttcacacagatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccgggtgagcataactttcttggcttttttgtttgattagtaggatagtagagtatgtgttggtcgagcagagccaggggcaagcatcgtacatgtagcagctgtatgcggatgagtgccactttcttcctccctacccccgaccctgcctcctttccttccttccttcctcccatccttccttcctctttccttcttctcctccctcctccctccttcccccgtccctccttccttcctttttcattgcttccttccttccttcgtccctccttcccttcctctttccttctgccctctctccctttttcctttcatcctccctccatccctccctccatccttccttctttcttccttctttccttcctataagcacctttttcatttctgtgctctgaatgaaatggttttctgtgtttattctgcaagcaaaacttgattcttgcaataaactttaagctttgcttactctttcagaaaggttttctcagggactttgggtgttgggttttacacacacacacatcaatacatttgggtaatttcaaaatctaaaaggaacaaaaaggcatacaatgaaaaaatctccttcctacccctgtttcccactcatgcagttctcttctccagaggcaaactcttacttgagtttcctgtgtgctctggagacacatcagcagatccctatacggtctttctcccgctttcttatggaaattgtaacactctgacatatactattccttgggcaagttaatcttgatgaagagactgggtgttctccatgctgaatgcctcacttttatgagctgccaagcccagttgtcccttccacctgacctccccctgtccagagacagatggccaaactgaatcataaaaagagggggaaaaaaagaaggcagtcgctgcagggctgtctttactccacactccacactcccagtccccaccgctgtgtctgagtcctggctgtggctgtccttggaacatttgcctcaccacgtgcctgtgtccccaggcgcctcaacctttcctctcctcattagctcttcccagttcagagggtgggaccggccagcacatctgcactgctgccctgccacacccacctccacctgcctctgggccccactggggaacacaggacaaatctgtgcggaggccccaccatgaaccgcccagacccgtggacccctgagactgactctttccagatcttgttagggtttcgtggctgctaggcaagtaacgaagcctcatctgtcccatgaatgataagaaattcagcatgtcagagtcagactctggaaaggcggggggataagaacacagccccagcagatggccagagcacccaggtgactgaaagtgctgctttgcagagctgtgtttgccacaggctcacagcccactaagtcttaagacagttttccttcagaataattaaatagccagcttaaagcaactcagaacattttcccctctgaggctgcacccatttagccaacatttgctaagcacccgccttcaaaaacctggtattttcatgtaaattatccgatacacagctgctatggaaacccccagtatcccacaggaagctccccagctcccagcagctgccggcccgtgtgagatcaggaggtctttaccagctgaacaccacgtgccgggtgtgtgctgatataaacaagcgtggcccactcgtcctgccctccagaggctcccgttccagtcggaaaaggacctgcccacgaagtttgcaacgatataagccacagtgtatgatcctccataatacagcgtgtgacagagcagcagaggagcgaggcagataacatgctgcaggccagaggcagcgggaagagccaggctgcaggggctgggggagccgtggtggaggaagttcaatttcagcctgtagatttctattagcccatttaataaataatgaagtgcctactctgagctaatcattgtgcaggtatttaggaaggacaaaaaaataattaggactcagtgcccaccctccaggggcccactgactagtagagaaagtaggcagatttttaaaaaattaatcatgggaatgtgataagtgctgggagagaggaatggatactttctcatgggaatcttggaaggcttgtaagggaaggcactctctgagccagctgtctaaagaagaacaggaatctttaagaaagcagaagggaaaagagcattctttcctgcttggagcaataggtaacagcctgcacatgcccaggcctagaggccaaagagcacagtgattccagaaagagtggggagaaagggtaggcagggaaggatgaggtaatgtgggcgcaggtgtggaggctggagagggaggaggttgtgggactgggaggagccagatggaatggacagcagtggcccagccaggagctatgctggcctcgtacgcctcgatgtcccttctattttctcaggggaggctctgcccaacatgccaagtccgaccacttgaaaacaagtccctggcttaacacagaccccagagagagtctccaaccctcctctccctagacaatggtagttgccctgtgaggggctgaaaagcagagctggagatggctcagggcctggtgttaacaaatgccttgagggctcctgttgtttcaaagtgagtctgcagggagagctccctaagtggacagcaggagggctgcagcttctctgcacattcctgctgtcacccccagagtcacctaggggaggggtaaggacagtaatgcaggttcctcacagttagcctcggtgcccacatggtactgagcatagtaaatgtttagaagatgctgcctggctagacaaaggggaagctcccgcccactagaaacttgcagggagccccagtccttgattggtcatttaattgattagctccttggcctggccttgaggcactgcttgtaagtacttcatgacctccattgcaaacccatgatgctctgctggacaaatccctccagtggccagtctggctgcaaggactctctgtctgcaggccttgccctgtgctgtcctgtgagagcatctgggccccacctgctgaagagagggggggtggggtttgccccgtttccaacagtcctacttctctgtttcagacgctgctcaggttcaacgccgtgctgaggaagctgctcattgtcttcccccacttctgcctgggccggggcctcattgaccttgcactgagccaggctgtgacagatgtctatgcccggtttggtgggtggtagccgaggcccatggagcatgggccctgggtccaaagctgggagggttaccgggggggctcctgcatcagactgtggcaggggctggtgctaggaggggaccttgttgggctggaggtgtcctgccagctggagaggattagggtgcctctgtttccatggctggggagccacaggagggatggagggcagcccttatgaggcgggtgtttggctcttgctcagttcccacataaggcctggtctagtgggccctgtgctgtggccaggtctgtggggtgagctggggcggctgaagtggactcaattcctgttgatgcccaggtgaggagcactctgcaaatccgttccactgggacctgattgggaagaacctgtttgccatggtggtggaaggggtggtgtacttcctcctgaccctgctggtccagcgccacttcttcctctcccaatggtacgtccatgccacaccctgggccagtgggcagctcagggcatccagaactggaccttatacccacatggtcatttctttcctcaggagccccactccacaatgttttttctacattctcaaagcctggcttttctccaataatacaagtagaggatcgggttaaaataggcacattcaaatatgtgaagagcatccactttaaaatatttaaaatgcagtgctattaatttcaattgctgatatttaatccttctcatttaattaccaaatgtgtattttgattagatgatagtattgcaaataacaatggttacagggtatccaaagtactaggaaatagactaatgtatttatgagagaaaggacacagcaggcccctttgctaattagagatttgggagcatgggagtaatatgggagccatgtggaggggtgcgggcagtgatcacgaccccccactcctggaggaaggtgggtagctgccaaccctgacttttgaccagggcttctcaaatgccaggttagctggcaattgccattcttccgcaggctcttcctgaagctgggtgggcccctgcctcactcccctctgcaatccagtcctacctttattgtcctcacccaggggcctgaattgccaagcagcagcccttcctagcaagctttccccaatagtgttttgtttcttaacttttcctcctctcaggctgagtgtggtcacctgtaaatagattccaaggacttggttttatgttttgatccacagggaattgatttattggaaatgaatctgcctttctactcacaggactgtgagaggtgaatgagatcacaggtgtcaacacacgcctgatgaaacaggatacacaagcagttctagttatgggagacagtgtcaggaattgttgtccttggcaccctcagcccctgcagaccctttctgcagccttggccataccttttagaggcttttgtgtgggagagagcaggtcaggaggttgactacccaaattgactcattagcttcaaactctgatgtcaacacatttgaatgagtcctgcctgctttagggcctaaagaggaccagagaagtacaccatagtccctggcttccagaaggtcagggagggtttcaaagaagaggctgtgtctttaagaatggggaagattccatttggtggggcaggaggaggagaacattgagggactggaaacacatgcggaggctgggagacgggaatgaccaataggactgggaaccagggggagatgccaattgctgacagaggagttagtgcaagaggtaagtgagaagggtaggtggggctggattgcagggctgtaactacagctgcagagggagggcttcaacctacagctgatggggaacaacagaaggttttgaggcatgaggtggcctgatgacaactctgttttggaaaggtggagttggcagggcagactggaggaagtgggaggctcggaggttagtaactaccccttactgagtgcttgctgtagaggaagcattttagtcctgacggtgatcccaggccctgagtctttactctgtgccaggcactgtgctgagttcatcttcagcacaatcctatgagacaggtattgttaccctcctcctcatcacatggttgaagtaggcaaggttcagagaggtccaatgcccaagatcacacatgaggaggccaggactggaacccaaggctgactctggacatgagcacctgacctctctacctaatgcctaatgcctctcctgctgggagccctttttagaatttaagtcttaaaggatggaagcccagaaggaagcagaagcaaggaagtggaagagaggtcccatggaaaggacagtgccaaggacactgtacagccagcccaatcctgaccccttttcttcatctaggattgccgagcccactaaggagcccattgttgatgaagatgatgatgtggctgaagaaagacaaagaattattactggtggaaataaaactgacatcttaaggctacatgaactaaccaaggtaagggaatgggtatgagtttggaggtgctggttagatccacagttggcatgatgttgccattttccttctatagaacaattgatatgcttatgcaagcaatttggttcccagttttatgtagggtcatcatccctgtgttataactcgtcttccaagagcatctaattccaatgtgtgttccctgctattcatctcgggcactgacacagggcctcagtgagaatcactccagctgagcatcattcccttttctgtgttctgtttctgcagagcatgggtcagcctcgagatgtctcagtactcaccacacctctgtgcctgcccatgtcaatatgtaacctcctagtgctggtagttttctcctaaaccatcctttgctctttgttccctcttcccctccttgctctcaccctgtctcagttctcagtccggtttcttcgtatcttgcagatttatccaggcacctccagcccagcagtggacaggctgtgtgtcggagttcgccctggagaggtgggtactctgcagaccacgtgtgaaaggcttccgaacatcagctcttgtgcctgcctctcctccccataaggcagagctattcaataggaacataatgccataatgcaagtcacatatgtaattttaaatcttccactagccacatgagaaaagtaaaaagaaaataggtaaaattaatttcattagtattttttattttactcaatataaccaaaatattatttcaaaatgtaattaatagaaaaccttattaatgaaatatttgacaatttctcgttgtttttaagtctttgaatctttacactcagggcccgtgtcaactgggacttagatgtgtttcaagtgcttagtagccacatatggctcgtggcctctgatggcagcccaggtctaaaattcctcccccagctcacacacacacttaccctggggcctgacattttagaccttcttgatctctagggccaggctagctctgtgttttctcctagtgctttggcctcctgggagtgaatggtgccggcaaaacaaccacattcaagatgctcactggggacaccacagtgacctcaggggatgccaccgtagcaggcaagaggtgagtatcctgctcctcctgtctcagggagtctctcacaggtcctgtgagaagaataggaagggtgatcatcagaccctatagtagggtggctctgaggccctgaaagatctgtacagagaaggaggcctcccagagagcatggcccaaaaagcccaacacatagacccaatggaaaagtgaactgaattgtgatagttaagagattcctctgttgggatggattcttggaaagacctgggaagcactaagtgtgtggttcttaatctcttagaggtcacggaaccttttaagcatctgatgaatatttgtagcctattcctataaaaatgcaccattgcttcccattacctccctccacacatttttacaaaacgtttcagggagtttactgagccccaggtcacatttatgatcctgcaggagctcttgaatcccaggttaagaacccctgtgatgaatgaagaatccttcctctgggttgagtttctagataggggctcatgcatgggcctttggggtagcctaacctgcattggctatttgtaggctgatatttggctttgccagaccaaggagcatagagggaaaactggcgtgtgcccttggattctggagggtgactgctgctctctgtaataaaatgtgtttaaacagactggtcccctatgggcaggacagagaggatgagctctcactcatctgcctctttcctggctgcaggaaaagcttgaacagtaaaacttcagcacacacaatagaggtgcccagaggaagcctctgccctggtttataagtggagttaggtgctgctgacatctgtccagcatctgcttgactggggcctcttcctctctcctgaaagccatcctcagcatggcccaatgcccagtgggcaggacgagtcctgagcacgcttcactggctcagacaggatgaatttgattctttggcctccatagccagccctactgggtttacagaaaagggacaggcaggggtgaagccaggtcatggctgagtccatctcaacagatccagcttcacctgcaagtgaccacgcaggtgacttcctcatggtgacaaaaggagtcatggcagggtagagatatcataccatggcaggggaaagatatcatagaattttccatgagcacatttatgagacatcaagttacaactgtgtccaagtgaggcacagtctgacatccagaaggtaaaactgagctggacgctagaaagaaactataggcttaagacacagaattgggattatatggtagggtagctcccactaatttggaaacgtaccctacttgcttccctgagtagttttaattggcccagccatgcctttggtggcttttgtcattgtggggaactgtaatggtctctctgtaccatcctatatcatccatcctttattcatagaccctaagctataagaagaaaaggatgagattagactaaatgtctatgtatagtttattttccatcttggcaatatattttttagtgggggtgaatatattagccaaagggagttggtggaacccaactcactctacccctgctccctgcaggcctctcgctgtgggtagttatctgactggctcctctttcattgctatctttgccaataaatacagatagagaagtttacttccatcgggacacatgcatcttttctagttacttcccaaatgtctgaaaattattgataaatcatgaatcattttcttaaacctgatcttccctctgtttttaaactcacatgtgaggtgatctgatccaaaatgaaagctgacttttggcgtaacagggattcaattaatcctagacatggaaacatggaagaatctgacaggattcagtttctaaccgaagggcccctgttttgattcccaaatatcccatgcatttctgaagccaaataggagaagagaagaagcagcttccttttcccgttggcagaagcttctccagccctagctctatggtcatccctccactccttgaaggatactcagtaattgctttttttcttgcagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggtgaaaaatgttttgttgtggccacataggagtctggttaattacaagcctgtttcatgagagtgcattctcttggagatgagaaactgaagcgtgctattcattcattcattccaacaaatgtttactatgtgtctactgtgtgccaagtactgttctagaaaccaggagtatagcagtgaacaagacagacaaaaaaaaatccccactctcatatctaacaaaatgttgtatgcatttatcctctgactcagcaatcacacgtctaagagtttatcctgaagatgcatctcccacagtgcaaaatgaatatgtataaggtgatccattgcatttgtaattgcaaaatgctggaagttacctaaatgtttagtcattgtagattggctgaataatttatggtacagacacacaataaagtcttacgcaactataaaaaagaagaagaaaagtctcagtaaactgatatggagatatttccagtaaatactgttaaatgataaaaagcaaagtggaaaacagaacatagagaacgctactttgtatgtaagaaagaaggaaaaacaagaaagtaaacgtatgtctgcttacctttgcaaatagaacgtagaaaggataaaccagaaaacaatgaatttggtgatcaacaagaagaaaatgggaagaaagaaaaatgggaggaaacagtacttctggggatatatttttgtatagttttaatttttggaagcatgttaatgttccacatattcaaaaaaaatcagtaagaatgggaagtaggcaaaaatgaaaacaaaaagaaaacctaacactgacagcaaactaaataaagtaacccaattttatttcaaataaatatcataatcttgcaaaagggggatagagctaacacaaacaactgctgaacacagtgtttgactctatatcctcattcttgggcagggtggagcgggggagaagaactacaaataatttctgagttctttttagtttgttttttatagtggtataggcaaagtgattctgaaaattttagatgtgttacaggattaaataaattaataaatgttttgatgttattgggacccagaattctcaccgtggaagaagggacttacaaatatggaaaagggaaaagcaagaaagaactgtgaggtcatggataggaaccggaggtagcactgggaattcaggaatatttatatgcttgtgtttgtgggtgcatgcagatgtgttcatgtttcatgcacataggcatgtatatatagacatatatttgcatgtgtgtatctgtcttccgaaaggctcaagaagcaaaaacaccccagtagccatgagcacacttagcactcaggcttttgtcttaataacattccccactaaaagtaaccctgattcctccaataaatgataagttccagggctggaatggcataggtataaaatgaacctggaatatcttatgccagaaagtaaggaagtgcttttaaaaaaaaaataaggggctgggcatggtggctcacacctgtaatcgcagcactttgggaggccaaggtaggaagatcgcttgagcccaggagttccagattagcctgtgcaacatagggagaccctgtctctacaaaaaattagcaaacaaattagctgggcctggtggtgcacgcctatagtcccagctactcaggtggctgaggtgggaggaatgcttgagcccaggaggttgaggctgcagtgagctgtgatcaagccactgctctccagcctgggaaacagagcaagactctgtctcttaaaataataataatataattttaaagaaataaaagtaactctgtacagattgcttattggttacatgggagaaacataataattttacaatggagaaattagacagcaccttaactgggtgatcaaaattaaccataaggggcagatggacatctcatgccccgagatgtgataccctgtgaaggacacaatttcacttatgtagaatccagattggagatatgtaacctgaatcttatcatgaggaaacatctgacaagctccaaagaaggaatattccttaaaaaaaaaaaaaggagactgtattcttcaaaaacataagagtcataaaagacaaagaaagagctatggaaatatctctgatcgcaggaggctaaacaggcataatgactgaatagcagacaatagactacatcttgtgcagaagagaaaaaaaatgatagaaggatattattggaccaactgacaaaactgaactatgaacagtagattaggtaaatgtatcataacattaagtttactgacattgataatgtactgtggttatgtaagagaagatctctattcttaggaaatatgccctgaagtatttaggagtgaagggctgtgatgagtaatttaccctcaaatgggtcacaaaaaattgtgtgtgagagagagaagggttttattagttaataattctatgaactatttttattcctatatgtttgtgtgagtttgaaactatttccaaataaaaagttaaaaatggagattacattctagtgggagggatagacgatctgtagataaataggtaaaatatccagtacattagagagtgaaaagtcctcagggaaaagtaacgcagggaggaactgctggggcagggtttgcattttgaggtagggtggcccagggagagcctgcagaggagagaacctgaatgaagaactagaggtgagagaaggagccacgtgcacacctagggaggaacattccaggcacgggggactagtatagaaggcagaagcatggtgagcttgtctccagtggcttccctagatcccctcctgcgcatgtgcacacacacctggtgtctctgtcatcgttccctcacagcactgtcacgatctgccagtattctgtttattttgactgccacctccccgcagtctgaggatagcagcaatggctgtgttcacattgttctccagtgcctggttcagtgcctggcgtatggtcagtgctccataggtatgtgtcggatgcacaaggctttgggtgtaaccctcttgacgggtgggatcaacaggtctgggactcaccatcttctcaaacagagccttcctcctccactgctagccatggtccaggacgctgggcgagacccactgtcttgctctttgtaaggctgaagtccatttcccaggcggctacacccaacagatgctgagcaggctgggccaccctgggatccaagacacagagagaaagagcccctgtctggcgcctgaagcacatgccagaggacaggagccagcaggagcctgtttcagcctagctggggatttcattctggaggcgtgagatctgggagcccaaggctttgaactgggggaggtttggggtgtttgcttgtcttctccaaatggcatttctttctcttccctaggttgcaaactggagtattaagagcctgggcctgactgtctacgccgactgcctggctggcacgtacagtgggggcaacaagcggaaactctccacagccatcgcactcattggctgcccaccgctggtgctgctggtaactgcgggcttgggccgcaccaagggcttaaaccaagtgctgggtctcttgggttggggaaataggttctgggtcggcagatttagaaactgcagcagtttggctttagtctggactgtttcctgtgttgctcattttgagcgatcagcccagtgtttggttcacacagctccggagaaaaacaagtcacggcacagccttgacttgggactgcgcacatcctgcgttcccaggatgtctcctgtggggccatcggctcacagccgggaagttcagcccactctgcggcctgtcggtgtctggtccccatacaggagcactgagctgggtcaaaggctcctgagctgagccaggccaggcctgaggccatgcccacgcagcccaaggatcatgagggcacaggacatagcgggaaccaaggaagtgacctgagtgacctccctgccttctgacaaatgtatttgcaggattttctttttttgaggagaattctgtcattgccttaatccactttaatcccctcgtgggctgaaatgggcccaggatggacgccacgcttctttactcttggatccacctcctgccttccctaccctacaccagggtacccctgtcttgctcaagtgaggggagtgactgtgtgcgccttctgtcagctcatcctccacaggggagccagcccagggggaagcagtaatcagaagggccagctcccagcctgtgcccccaaccttctctccaccccccaggatgagcccaccacagggatggacccccaggcacgccgcatgctgtggaacgtcatcgtgagcatcatcagagaagggagggctgtggtcctcacatcccacaggcaagagattcccagggctggggaaggtgggtgggaatcctctcctgctcacctcctctctcctgccccacagcatggaagaatgtgaggcactgtgtacccggctggccatcatggtaaagggcgcctttcgatgtatgggcaccattcagcatctcaagtccaagtaagcagatggtggggcgtgccccttgttgccttctgtggatccacctggatcctgtgttctccattgacacttggaagagtcctgctgctccgtcatcccctggggcagaggcaggtggtggctgggcctcattctccagcagcagatggagaaggccatcatgctgataagaaactcctctatattggcctaatttcctgtggtcgaagactcgcccaagtctctggatggggcatctgatcaggatgcatgcagagcctggctgggatgagggagggctgctaccactgcctcaatatttcaccacttatctcaacagatccgggacctgtggcctatttactaagagtccactccaatgtaggaatggttaggagaccaactgacttgaggacccatctttgtttttagaatattgtatgcttttgagtttgaaaaaagaccatatgttatatgacaaaccaacaatggcagtaatcttgaataggattatccttatcctgtacccacacattgtaaactattgtagataattccttattattaagagtttgcatgccaaagctaacagtttaagattatcagcatattgccgtgctcattcacgttctgatatgctttataacctagaaaagagcagagttacaattactcatttatttaacaaacacttattaagagctcagaatataagtcactaagctggttggtgggaggaacagcacataacccaccttatctatgctgaggtgcataatcctgatgcacccacaggagggtgttacacagaagatgtcatcctttcatatgtgtcagagcagataaataattgagagaaaggtctaatagattagctgcttgtggcaagtggacgtttgacccatgatttattgagcaactacaacttggacactgcatagatatctatagaaatagcagcatgtcaggtcaccagacctgtgtcagcaacttcctgtgtccaactgctggagaaagggaagtctcctattcctttccctccagctccttaatatctccatgatagagggggtgagaggggagtgttccctgtgtggagggatggtgagttttctggagctgaaaggtaaacagcctttctcctctgcatcttactgcagaggagaacagccctagactgtggaggaagctttggagtcagttatgactgacacaggataccagggcatagggtactgacacccgctagccgtgcacacactctctggtggaccatcactcatccaagagagggtaaccagccatcctgctgaaggagaaagaaagcaccaatggcccaagccctagcagctccattgtttcaggaagcttcctcagggaagtgctgccttcccgagcctttgctcccacctggcccatcagcccttaccaccactcagtatgcactggtccacgtgtctttatgggcagtcttgggatccccacactgggctaaaactacctttgacggccaggtgcagtggcttacacctgtaatcctatcactttgggaagctgaggcaggtggatcacttgaggtcaggagttcgagaccagcctggccaacacggtgaaaccctgtctctactaaaaatacaaaaattagatgggcatggtggtatgcacctgtaatcccacctactcgggaaactgaggcacaagaattgcttgaactcagaaggcagaggttgcagtgaatcgagatcacaccactgcactccagcctgggtgaaacagcaagactctgtctcaaaaaataaaataggctgggcgtggtggctcatgcctgtaatcccagcactttgggaggccaaggcgggcggatcacttgaggtcaggagtttaagaccagcctggccaacatagtgaaaccctgtctctactaaaaatacaaaaaaaaaaaaaaaaattagccgagtgtggtggcaggtgcctgtagttccagcctctcaggagactgaggcaggagaattgcttgaacccaggaggcggaggttgcagtgagccaagatcatgccactgtactccagcctgggcaacggtgagactgtctcaaataaaataaaataaaataaaataaaataaaataaaataaaataaataaaataaaataaataaaactacctttgacttcagcaagtacgattatcccacattaccatgcagacatttgatctctaaaaactggtatcaaatgatttctccagggactaccatggtttttctctcctagttttcagtatgtacacaggtctatggtatgggcctttaatccccagtatttctttttttgttgttcttgtttgggtttgtttcttgtttttcggtttttttgagacagggtctcactctgtcacccaggctggagtgcagtggcatgatcatggctcactgtagccttgacctcctatgctcaagtgatcctcccgcctcagcctcccaagtagctgggaccacaggcatgtgccaccatgccctgctaattttcgtagagacagggtctttcttgttgcccaggcttatcttacattcctgagctcaagtgatcctcccacctctacctcccaaattgctgggatttcaggtgtgagccaccaagctgagcttaatccccaaaatttctgatgagtctactccttattttgggattaccttaggcccaaccactaacagaggcctgtcctgcactgtgtgcatcccctagatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggcaagttggccctggggcaccgagagctgagcaaagactggtccagaacacccagtgtgggttggaattgccataagagggaggcataacattcccgatttttaacaaactcttgccctctgtttattggggtaaaagctgatatatcagaaattgttttctaacaatattttttagtcatcaggaaacttcattgattcttttttttacattttccttccctgtgatgctatggtgtgttatttcattcttgctcgtttgtggtggtggtttttccttcaaatcagctttattgatgtgtaattaacatacgatgaaacacaggttctttgggaggccaaggcaggaggatcacttgagcccaggagtttaagacaggcccatgtaacaaagtgagactttgtctctacagaaaaaaaaaaaaaaaatcagaaaattagccaggcgtggtggtgcatgcctgtggtcccatctacatgggaggttgaggaaggaagattgctggagcccaggaggtcaaggctgcaatgagctgtgttcataccactgcactctagtctgggtgacagagcaagcccctgtctcaaaaaagcaaaacaaaacaaaaacacctattttaaatgtacagtttagtgagttttgataaacgtgcattccatgtgtggtttttaaaaatgtaatcacattttttattgcggtaaaatataataacataaaattgaccatgccaaccatgtttaagtgcacagtgcagtggcactaagtacatttacattgttgtgcaaccgttaccaccatccccgatagaactctttcatcttgcttcagtgaaaatctgtgcccattaaacactaactcaccacttactgcccccctcgcccttggcaactactgttctactttctgtctctaaggctctgactactatagatacctcatataagtggaatcatacagtgtttgtccttttgtgtctggcttattatgcgaggacttagcataatgtcctcaaggttcatccgtgttgtatcatgtgccagaatttccttcctttttcaggccgaataatattcctttgtacgtatatgtgctacattttgttcatccatctattcattcattgatagacatttgggttgtttctgggttttgtgtttttatatatgtttttttaaaaataaacatctttagagacagttcagtaaagcagtggaaacagggaagtctccatttaacccctgaggatctggctcacctgcaccttctcatcagcattaagcagagggaggcacgagcaggagccacctgcacactcaatgaggagctgaacagggatcaattacctttttttttagttattaggatgctgctagctgagaatctgccttgccttgattaccccaatgtctggtgcccaagtcccttgagtcctccagcaggaactcctgtggcatcactcaggagtctagtctaagaagctagctctgaccagggcagtggtggccaggcttctgtgagtgggccagcctcccccgggtaggacacaagccataccagcagggctgtatgtgaactgtggaaaatagagagcaaagtgggtaggtgggtgtagggtgctgttttcctggaaatatctacctaatctcgctcttctcttacctctaggtgtttgtaaattttgctaaacagcagactgaaagtcatgacctccctctgcaccctcgagctgctggagccagtcgacaagcccaggtacccctgctgcttatgcagtccacagcttgaggcagttccttggctcagagcccagctggttcactgggcttgagttgctccaaggctcagatatgcctcctacagagagccccacccacaccacggtccctaccaagtccccaccacatcctcatcacatccttgctaagtccctgccactgtgtgttctgtgctgaagaacttttcattcagtagttgtaggggttcctattgtaatcaggaaaccatctggatagcatgggagagcatttttgaaaagaactttcccatgtttttgcttacagcaaaaaagcttggatttggggaataaggagcagagaaggtaatagagaatattagaatgttttgggtgcttgacatctatgtctggacatgtgtttgagtttcaagggaagggacttaactggcacatcatttcagtgtcagacacatttggttagatcaaggaatagcatctgttgtaggaagagggctctttgttctttataaaaattacaagaagatggagaaagaagcaataggaggtatgtctcctggcttgtgataactcttggaataggtgcttgtaggttcctgccctggcacagtgccccatgtaaggagcacaccacccaagaaggagagagctagagcaagtactggaggaggcaccagcatcccaatgccttggcttaagcctgggattgtagagggatgaattagccactctcttctgacttacctggagagtaaatcaaatcaaatcaagaagcaaggatatgcaaaaaccttatttccccataaagtttttattctgcccagtttctggattgcaagaaaaaccaaatacagctaatgattgaaacactgctgtctaaagcagtgcttgtgatgaattttttcccttcctcttgaccagcagagacctaatggctacttggcaaaactgactttgtcttcccaccccttacctgccagagggcccagaaatgcctaaggctcctttagttacagaaagtttgcttttactgagatcttccagccactgattcccatttatagatctggtgattgctgttgacatcagttgaaaattatttttaaaaaccacttgcagttgcaaatcctttttataactctgtaactcagaatatagaattgggtagcaaaattgtttcccagaattaccaatggtctccccacccctgcctggcatgttccctcttaaaggactaatcccaccacatcacctctgggccaggcagaacatcaggggtgctgatgttctgtgatctacagcagttaattccaaacttttctcccttattggatgagatcatttttctattgtgttttttacatttttgttcacaaagattagaaaacctgcaacacacttattggcatatttttctgataattttcatccaaaacctaattctgactttacaacatactatctttacaaaggtttgcaaaaattctttcatatagcattgtatatgtctgtcatgaaataatagtaagtatattattgtttacattataccacttcaaaataatttcctttaaagtattcttcaaacaagaaaaaggcaatttctctcaagaagttttagagagaatttacaacttgctcctaagcaaatgtgagaacttcaggaggttcatctggccattggctttacaactccaaattgtgagccaggaccacacagatatttctctagaaatcagcgtttgcttaccaagaacatttttactctccaaaggactccatcctggaaaacatgttttgggataaggtcttatgcaatcttatactctgttattaaaaccagtgagggtcaaggtgttaatagattaagtagtgacagatgatcagacaacttagaaacatcctaaataggttaataattatgtgaccatcgcatgtgcattcccaaattaggaacaactcagatcaatttctaatccttattcttacactgttccagttcccccatataactcgtatctttgtgttagtttcagaagtttctgaagtaccctcagccttgatggggatcctcgcaccacctcaaatcctgttctcagccctaagaactgtgttagtcatcctcttaagaggatgtgtgattttaaatcagataatgggataaaccacatttcgtctagactggtcaggcctttgtccagtcccctcctcgcccacactaccccagctccacagcgggcattggttcaggaattcaacccacactttataactggagacagtatctctccagttaaaaaggtcaccttggtgtccgcttctcaaggaacatggacatctttattaatcaaagcccaagctttgatctggagcctaatatcctgcactccagctctcatctctcccctcccccagtcacactttcatgcttcccagagccacccctacaggaagtggtcaagggaattctatacctcagggctgacctaaattaggatttcttggcttttaagataatggtaactttcttaagctaaaaaagccccaaaagaccctgtaagagcccttggaaacagcaccatgggtgtagcttccccccaggatgtaagcatgtatgcacacatctcgtatgtgtgtctttgtaacaaatgcctggatcttagtaccagggagacctgattcatagatttcatagagaaggagagaaagatggcccataacctgggtgatctgacagaatcacagtgccctcagctgagtgcccttcagaaattgattgacaactgtttagcttttgaaatctaaaagtagtacagcatctcagaaaaccaagatgacgcgagtccatgtgatctccttccacaggactgatctttcacaccgctcgttcctgcagccagaaaggaactctgggcagctggaggcgcaggagcctgtgcccatatggtcatccaaatggactggccagcgtaaatgaccccactgcagcagaaaacaaacacacgaggagcatgcagcgaattcagaaagaggtctttcagaaggaaaccgaaactgacttgctcacctggaacacctgatggtgaaaccaaacaaatacaaaatccttctccagaccccagaactagaaaccccgggccatcccactagcagctttggcctccatattgctctcatttcaagcagatctgcttttctgcatgtttgtctgtgtgtctgcgttgtgtgtgattttcatggaaaaataaaatgcaaatgcactcatcacaaactatcctaattcacagtctccctggtgtgcaccacctagtatagttttagacattctttagatgggtgcatagctccttgtcagtcccatgcacttctgtgagtgttactgcctcaggactgctcgttctggcaagattctgcaacactaggttggaagtgaatggactagtcttaatgttccatgtcaagtctttgtagagtttgaagaaaacacccaactagtaatgcctgtaaacattatccattgtcagctgggtattactgactttaagtttcgtgtctgtttgcccagcttatttgagtgtttacctcacaagtgtaattaggacaggagacaaagaggcatgcacaggcgagagttggtccttggttggacgtgagacccgacaggactttgtaaccatttgaagaggtcaggacctcattctcatgctgctgtgccttttctgcagtgctaccatgtgcatcttctgcagtggtgttagaagggaatgaaggccgggcgcagtagctcacgcctgtaatcccagcactttgggagcctgaggtgggcagatcacaagatcagaagatcaagaccatcctgactaacacagtgagaccccgtgtctactaaaactatgaaaaattagctgagcatggtggcacatgcctgtagtcccagctactcaggaggctgaggcaggagaatcgcttgaacccaggaggcagaggttgcagtgagctgagattgtgccactgcacaccagcctggcgacagagcaagactccatctaaaaataaataaataaataaaaaggaatgaagatggacatctagttcacataaatgctcatcaagattcagaaaataataattttaaaccaaacatttccagagactgtggatgaggagaacaagtgggtttccttgtcagggcagtctcccctccatgacctagagatgggcttcgtcgtaaacatgctttcattatactgaggatagactccagagtcggggtgaggcaagaggaaaaggggagagatgctgaggcccaggaactgttgctgagaagaggatgagaaagaaagtgagcacaaggtgctaaaatttttgtttcagctgtgcttgtttagaggcagacagagggcaagggctacacaactttaagtcctgcacacctcctggcttgccactttgcaccttctagatgctaggcaaacaactgcccatagagactataaaactactttggtaaccccgcagcttcatctggtgttgactttttcttttaagttaccaaggaccaaaactgtaagaccattttagctagggctaggatagaggttgggaaagcccagcacactgcttgcatcacactgctgcaccctggctggtttcataatttaaattagcaactgcaatatcacaggaaagaagactacactcttcggggctgcttaggaaaagaaaactaaaaaaagactatgtaggggaggtggtttagcagccattctgtttggctgtgagggtttcggaaaggcatcatgaactgggaagagtcgatgcaggtaaaatctgcacacccccttaaggaaaaatctcagtttaccttttgtctccactacacctgaggtctgtgtcttttcatcctgttttttccagctcctggcacacagaaaatgttcaactaatacccaccaaactgaaaacccagcaaactatgaaaactcagcaagaaaaatagacagaaaagaagtgggtccaagaaaatgatgcctccaaaaagcagcaaagggcaagtggagcaggaggatgccgtgctttaaaaacagccacaggccgggtgtggtggctcacgtctaatcctagcactttgggaggccgaggcgggcggattgcctgagctcaggagttcgagaccagctggccaatgtggtgaaagcccgtctctattaaaatacaaaaaaaaaaaaaagaaagaaagaaaattagccaggcgtggtggtgggtgcctgt

SEQ ID NO: 7 - RNA, 7913 nt, Homo sapiens
  source 1..7913: mol_type = other RNA
gtaacagttactgtctgtggtttaaaaatgaggtgtggagcaaataaacaggttggaagtgtggggtggggtggtggggtagggtggtggggcagggtggggggttgtgagcagtcagtgggcttgtcgccgattagcactgaagcagtgtttagctggacggcctttctgtgggcccctctgacagtgcccttcccaggaagatgtgtttctctgtcctcagccacatgaaaatcttttgcctaccgtgcctgtcaatccattgcctgcccgcccctcccccaccccccgttttacacctgcctgtccagtctaccgctctctagggcatccacgctgagcagtgggaagaactttaagccctgaagagcaggccaaaggcaagcaagaaccccctcgaacagcttcccagcttagtgaggccttatttcattgattctctgaggcacattgttttttcacatgttagcatttctgaaattgggatgcagctcacgatcaagtcacagtttaactggacacattatttttctttcttagtggtgcagaaaagtaacagtgtgtcttacaattgactgcgtcctagattctgtgagatgcaatacgttattaaccatcacgcacatttcctgaactctttcaatgagcagacaccagcctgggttagactggagccctaaaagcacgacacagattccaccctggactggcttctgttctgcctgggaaaacccaaagtacgtttggagaccaagagcaacataaagtagcataggtggaatagtccatgagaagtgcgagcaaaaggtgccggagatcagagaacaccaagactgtacttgtaaatgacaactggctttgtgcaattttttctgggaaaggataaggagtgactatagaactgtaaagaaagaatgcactttgctacagccttgcagagttgtgcaaatgccgatgactaaaggagctgaaagaggaaggaggggataagggatgggggctgggtaggggtgagattaggaccctgggagctgcaagccactggagagatcaggaggaaagggagggagacctgctttaggcgagaagagaacagtatttgttccaaatctcggttcagaataagttcatgtaggtgatggggccaactggaacaggtgaaggcctatgaatgagtgtctcagttagggtctccttagagtttaatatgaaaaggtgttagctaagtacagagctggtacctgagagagtaaaaggaaactctaaggtatcatggaggtagcaattgcaggacacagctcccacccctagggctgagagaaccaagggaagagacaggaattattaagacttggagcatagatgagaggtctgtggagctgacattaggacttgggaggaaggcgtgcatggaggctgctgctggatctctgaacctgacctcgggtctggacccctgaggagaaagccctggcaggttggtgcatgtggggccgagggacaatagcttaacaaccagcataaaagagagcagcatgggacacgcttcaaccatgcgcatggatggctccaaaacctgtgtgtggctggcccaggacgcagggaggctgcagggggaagagacaagttaaacctgacttgtctgggaagcaccattgtcctcaggtcactttcctctgtcaagcctggtgctgaagttatctgttgtctccaggggccaagtattaagagtaatcagaaactcagtcctttcttctaggagcttcccttcttgcatgaaaatcctgataaaactggaaaaaaaaacctcatgattaaattttttcatgtattcattctttccttctatcaaaaaataatctccaggcaccgtgctaggttcattggtatacaatggcaacaagacctcccagcccctgcctatgtgaggcatctgtggactgcggaggaaaatccaatatgccattgttctctctttcccataagaaattacaattctcagttcattttattctcactgtgctctttgtgaccctcaaagggggtcacatgataacaggactgtagctgctggcctaaaatgagcccattcctgtggcgctcatgtcgctgtgacagagaataaccctgttttcagaatgctctggtgccctccctctcaatctggcctttcgctggcatgggtgggcgactcctgctcagggactctgccttctccacagtgtgctcccagggagatggagccactcgggctgagggccttggccagggcacctcccagggctgggcctggtctgggctggcgttcactggatgccatcctgatggcctggaaattgagatttctgtctggcacgcctcccgatggctccccacctgctaccacattccaggagcttccaggatgtctgggtaagacagaggcacccccaacagattcagtagctctgagagggatctgtggctccttcctaagcttgcggttcttctggaaacttctgcctctagaagatggtccctctaagaaaagtacaaccacccagcccataattcagctcccaggttttccctcaaacctccatgtctcctgtaagcagagcaagagtaaaatcagataccaaatttcctcattcctcagctcccaatccctaagggcataagatgaaaatcttcagatctctgctttcctccctctttttttcttcctctgttaacatttgtcaagtgttactaagtgtctggcactgtactaagtgcatcacctccctgaactctccgaacagttccacgagagaggcctctctgtgatccccccggtactgatgaggtcactgaggctccagagaaggattagtaactggtggggttggacctgggattcacacccatgctgcgtgacccaggacaggcaggcatggccgttacaccacactgacccccgtggatcgagatctatccaatagtctggtcactgatatcactaagatagagtggccatataatttatcatccaatcagggcagttttgcaagtgaaagggagcactattaataattgcactgggacaataaatgtaaaccaacactggacctggaaaactgggacgtgtgtttgccctataccaaggtaagctagacacagccactgccttcatggagttcagaaccaggcaggggcggctcccacgtataattactgtgcagcacaacgtggagaccgtggagtagaaggaaacacggatgggaggtgaggaggaggtctgtgagctcagaggaggcaccggggctggagagggtgagagaagacttcccaaggagttcatcctgataacgtgcattcccaatgacgagcgctctctccactgcacaagacaagtatacatctgcccgtgttggctgtggacctggcgctgtgtcagggagggtttatgaagatcactaggtgggtctcttggtgtcatcccttcatcccagcttctgggttaggatggatatctgtgggggggcctgaggactcatgaaagtggggcgctaatcatgttttggacaccacaccctggagcacctgggacagctgtggcctttgtcctgggttcagcatcaagccgaggatgtggcaagtaaagagaggctgggcaccaactccagtgtacccaggctccgggtcatgtttgtccaggctaagaattctgtcctggttctcagtgcagaaggaagaatcatggggctcattttaggccttggctgccttctgttaaattgaaaacagagcaggaaggaagaaaatttaacaggctcagttctaaaacaacaagcacaactgtgcccttgccagaaacccctcctccccatgttgattgaatggtaaagagaggaggggaggtgagagggagagagagagagaggaagagagagagaaaggaaagaaaggaaagaagaagaaagaaagaaaaggaaagaaagaaagaaagaaagaaagaaagaaagaaagaaagaaagaaagaaagaaagagaaagaaagaaaggagggagggagggaaggggaaaagaaaagaaaagaaaaagaaaaaaagaaggaaataccagtttgggaaaaaagaattttccaccagcccttctgagccttggctgggcttaattaaagttacagacatgtgtaaagggcagggtagggggagtctgagctgctgagaaaacatgtttttaattatactgtggaatttctccctggggtatgcctgtacgcagttaagcgtcaaggacagggatgccgctctggggaggggaagctgagcatgattttggaagccggcagaagaggctattgtgaaaaccagacctgtcaggctaggaaaagaatggctggtggtctttgaccagggagtgacgcgtgaaatgcagcaaccgcccccgccccccgccaaaaacaaacacactctcacagagttagaacaacagtgacctctcaacaaatatttttcaaagattaccaaccaaccattacctagagcagcggttctcaaccttggctgcacggtggaactacctgagacgtgttaaaaagaagaaccctgatgtcccatgccccaagattctgatgtagttgatctggggtatgatctgagaccccggcatgttttcagcctgcagccacatgagaagtgctgacctaatcaacaggggtgatgatttgaggggcggggactataggcaaaaaaaaacagcctaattcaaggatgagaagagggcacaggtgaggtgggaacagtcctagggccagacaaagaaggaagggagaaaggaggtgctgatccctcccctactcctgagaggaggcctttaagtcaccgtgccttgtggagaccagattcttcaaaaatacaagaatgagtgagtgagggagtgggtggatgccaggagagtgcgtgacaagccttgcaagggaggatgacaatgcactagcttggtttggaaattttacccctggaacaggcaggccaagctggctggtcccctccctgatacacagccctccctctttatatatggagcaggggacggtgtgtggctggtttcttagcaagcaccatggttccaagttggcaactggggagttctgaatccaaaaaggagggagatgaacgtaagtggagggcaggcctacaaggttgcagataagcttaattctgtctccttactcttctgcctttgcaacaaccctgtgatcttgcgacaaccctgtaaggcaataacaaatggctcatgtttattgagtgttacctcatgccatattgtgctttcgtgtttaacacaattgtctcatttcaccctcacgactgctctgggaggtaggtcctggtatcacatccatttcacagatgagaccatttggcacggaagagttgagtgggctgcccaaggtcacatagctaagatggaacaggctggataggaaccccagtaacttgacctcagagtaaccttctcttaaccctgagtgtacactgtaggaaaaatgagcagtcccatttcagagaggacaaaactgagactcagaggttaagcaagccccaaagtggttgttaacccagatcttcccactaactcccaaatcagcatcagtgtttaacgtaccagacctctcccagatagatgttgccgcatggaagacagccgatctacgtgatagaaagccaatattgcaagcagtcgtctaaaggagtcaaatgtgttggatttgaactggatgtctcatttctttggtgaagacactggaaacaacttccaggtttcatcaattgctcctatcactcaacgttgctatcttactgaacttgttccccagccttacccactgatggaatgatccagaatggaagacaagacaccaatgtacatgaccctgggggaggctgtttcttaaatctacagactgttggtgacctgagccccatgtcaccaaaggctttcctggagaagcctcctagaccagtcttgacaaaggctcactcattccgtggatatttattgggcacctattatgagttctgccccatgtggggtgctggaatcacagtagtgacaacgacagatgaggttcctgtcctcaggaagcttactgcccttgagggcttcacttacttggaggagtgatgaacctgaagtgcggtgtgtgttaagaagcggaagtccagggccaggcgcggtggctcacgcctgtaatcccagcactttgggaggctgaggcaggcggatcaccaggtcaggagatcgagaccatcctggctaacatggtgaaaccccgtctctactaaaaatacaaaaaaattagccgggcatggtggtgggcacctgcagtcccagctactcaggaggctgaggcaggagagtggcgtgaacctgggaggcagagcttgcagtgagccaagatcgtgccactgcactccagcctgggcaacagagtgagactccgtctcaaaaagaaaaaaaaaagtgcctcacggagagtctattcttttcttcccatattgtgtgtgtgtgtgcgcgcttcctccaacacatcctccctatatatattttgagtaaaacatcttgtaaaaagttacagctacataatcaccacctgtccctaaatagtttttgctttttctttcttcaatgcacgatcattttcccccatcaatttattttttagtttcttataatcttgttgccagtaggctgttttttaaaaagcagaacatggtttgttcttactagcaggaaaggagcatttattgagcctctgctatggtgtcttttattttgctgagagcctatttacatttctttgagaggaaaacaacaaagggttacatgaaagaccatgtgaatagcccctagctgatctattaaacttgctattccccggccagctgcttcagatctccttcagatcttatgtgtttccttcctaaggtccctggagtaagggttgcatagacctattctactctccaactcacatgtccctctccctcttcctctccataattccacatctccaacccccacccctatgtgcaatgccacagggtgtggactgccacagccactggatctgcttttggaatcaagagtccttaagctccaaatggaaccgaaatttaaataccaactttcaaccatatgttaacatcagcagcctcttccaatgtaaaaacccatggcagtgtgccctgctttgtttctttaagcaatagaaacttgaaggaagcatgttggtaggccagatttttgttggctttgcaatggatcacagtcatttattcactcattcattcactgattcattaaatgaccacatttgcaagggcaaggtaatggggagggccagaaaggacactggccccagaaacaggaggctggattttggttctgatgctgccactgctgatgtgacactgcacaggtcacctgcctcctctgagcctctttccttaactgcagagtgagtggctacagagaaatctttactacctgttagatcagcattacctgggagcttgttagaaatgcaagctctggtggggccatactgaacccaaatctgcattcatgtgcatagtgacagctaaaatgcactgaagcagatgatcttgatgatcctttatgaaagtctcatgctaatgcagttttctaaaatagaggcagagtggaacccagatggacacaaaatctggttgatataataaaacaaggtagagggtgtatggtggggagggggtaaaggaaggaaactgtttaggtaaagataccacaaccaaagtcctactgcacacatgggatctgaggagggctgtgtctgctctggttacgttttctataatctcttagcaccactgaactttctctctttttgttttgtttttccag

SEQ ID NO: 8 - RNA, 1393 nt, Homo sapiens
  source 1..1393: mol_type = other RNA
gtaagcatagcagggtagcttgggcaagccctgaagagactttggtctgggccttttgtctagaaagatcttggggtgggagtgtggggatcagatctgcttatcatcatttcatgtctatgatgcatgtaacagatttatcaatgttacacaaattataatttttaaaaagtctttagagacagggtctcactctgttgccgaggctggagtacagtgttaggaccatggcacactgcagcttctatctcttgggctcaagtgatcctcctgcctgggcttccaaagtgctggaattataggcatgagccactgctcccagctaatttttttgttttttgtggagacagagtcactacattgcccgggctggtcttgaactcctggcctcaagtgatcctcccacctcagcgttctaaagcactgggattacaagcatgagccaccttgtccagcccaaattttcatgttttaatcctacacattctaagcaaatacttgtgtgtagttactaagggactgtgcacttatttttgtttgctttgttgttgctagtttttatttttttatacctaaactctctcgttttaaagagaacagatttgtagatgagttctcgaaaatatttcaggaatcaatatagagaatatgttatacatggtgccagagaaaaatgaggacaagagatgctatacaatcgtactgaagaaaaattttatttcttggacccctgaggtgtctgcagacctgaaaggaacctagtgagagcctcttttacactctgcccctgtgggaaagccttcacctggtttccggccctctatgtggtgaatgtggaagcctcaagcgttatgcaaatctgcccagtcctctattcttgatcttcaccttctcgttcatgagtttcaggccccagttctgaatcagcctcctgtccatcagactcttctttacctctccccgaggagcccataacctgcagccctactgcatgcttggggtaggtgctcagttcaccgtggttgaaggaatagacgagcgtctgctcaagcagcagcagcaactgcgtggagtcttcttgaactaacactcctatgcccctctcggcacaaaatgacgtgtccccccttgcttccccttcacatttccacccatgcctattacaacatccgtctgtctccccactacaccgggagcttgagagaagaggccatgtctctagcacccagcacagggactggcacacatgagatgctcctgcttcttaaatgctgagaatgaaggaggacatcagaggggcccgggccccttcccaaaaaggccaactcctaggtctgcatcctgcttggtctccatgactaatcccgtcttgtcctcattttctgttttaaag

SEQ ID NO: 9 - RNA, 2721 nt, Homo sapiens
  source 1..2721: mol_type = other RNA
gtaagtgttgagatccctaccatgcaggggaggaagttgcacaccccttcacgtgctgaaatgcacacgtgcgtgcacggagcatggagcactgagtgttcttgtggctttgctgagcccctaacctcttaggagcagagcaggtttcctctctggaacattctgttaactgtcagggcacttggggagaaatctccaagctaaggccacgtgcacaaaatttcttggtccttatatccccagaatgtgacctggagtctgatggcagcccgctgcagagatgtgtccactgccttctggtcattgacctgcttgggtggagtgaatcattgtaggagaaaaactcagttccctcaccctgatcaacctggacagatctctcttcctttaaaagctttcttggacatctaagggctaggaaaaatgtcagggagcattgggaaggtaaatgaagtcaggtttacaaagtcaagtttacttcttgggagaaaaatacaatttccaaatcctctgttataattgccatcggccccctggagtggtgagatctcggaatatggctcgggtgcagtggctcttcactgtgggcctgcaggctattctgaaaagctgatgaaaaccaatgacccctcttccaagaaaaatggccacataccaaacattacactgtacatctgatttcagggaattgtagatgccaggttagtagcctcaggtctagggtcaaaattcaagtcgaatcccacaggaagagggtctgccttcggaattccctttcagagcattgggagaacatcatgggagcatattctagagacagaggcttagggtgtggacagggccatccctcacccactgtgctgaccttaagcagcaccttgtgcagcccatacctgaaggccaccagcaaaggcctgttggggagcaggctttacccgacctgtataaacaccaggctaggtgaaaactgagatacctggttactttagttttttccttgggggagctcagtatgattcttccaggagaagcctgcttttagactaaaaagaaaaaaagtttgataggtcaacctaatgattggaggtggccttccccactgtgaacaaactatggctgcatgtgccctacaatggcagagttgagtagttgtgatagagactgtatgatctgtaagcctgtaatttttatgtttgctgacccctggattaccagatgatagaagaggaaacatctgtcttcctagcaaagtcaaggaagtggcatttagcaggactcatattgctgcaagcactgccttgcagttttagtttacaactgcactttcagcttaagaaacacctgcccatccagagagatcgtgtggggtcacatggtgggatcagggaggcctgaagacagctcagtggaggctgcatggagctttggtgggaacggccctggcagtgtctatagatgttattgcggaaaactgaggggtgggagttggagaagggggctccagactctagctgtacttggcatttgaacccggaaagttgggtttcatgttttgcactcacattatgagtgaaatattggcttattcaaggttcttttgcttgcaaggcacggaaacccattcaagcaatcttaaaccccagaaggaaatctatgatttggatactagacattctcacagagccaagggcagcaaggcggggctcaggagaggcaggccaagacctggagagctgtcaggagctgcttcctcaactctcttccatctgggcctgccagccctggcctctgtatctactccattcacctctctccatggaccagtctcccctgctcctcaatgcctgggctgccattgttcatgcaattcacaatacctcggcctgggcaatcagaagctcatctctgaacaccatccaaattcctgggaacaaatcgggttgacccagctttattctccctgtcccatcagccttggcagaggcgtgcatgtgcatgcgtgccaatgtgtgtgtgcagggaggtccttgtggatgaagcatggctgtcagagcctacctgcgtgaatgggtggaagggcaggtctcagagaattgggtaaaaactggataaaccctccagtgatatccaccaatgtcaccctgtttaaggcttctctgggcaagagacacacagagcatgggaccgagaggcgagcagaccctgccaaaactgggagactgaatagatcgctcaccatccttgtcagttagcctatatgtacaaggaagtaaaattatctctttctcctgccttggcagtattgtaaggatactcaatgtagtagctaggccagacacatagtatctttaaatatagcatgagatggccaagcacggtggctcatgcctgtaatcccagcactttgggaggctgaggcgggtggatcacgaggtcaggagatcgagaccatcctggctaacacgatgaagccccgtctctactaaaaatataaaaaattagctgggtgtggtggcgggcgcctgtagtcccagctactcgggaggctgaggcaggagaatagcgtgaacccggggggcagagcttgcagtgagccgagatcacgccactgcactccagcctgggtgacagagcgagataaaaaaaaaaaatagcatgagatattattactgttataaaaataacagctatttccttattaatgaggctttgtccttacag

SEQ ID NO: 10 - RNA, 5434 nt, Homo sapiens
  source 1..5434: mol_type = other RNA
gtaagcatgactgcagtgctctcaagcatcatttccctcacctatggagagactgaagatataggaaagaacagggagagttggtgaaaaatatactagcggaggcaggaagggatggggtctggaggcggcttgaacatcaccttggtgaagatgcctcttcctccacagaagcctggaaggtaggaagttgggaaggaaggcaggaaaggtctcatccacgttaagtctagagacagaaagaatgctaagagagatggcactatgggaagtatgaggctaggtcaagggctagaagcaggggagacgagtttacagagtttcgtaaagatatagagcaactctcacagagttctagagcgagagctaaccaggaacatgaagcagcaaggccaactatcattaaggagccagggaggtcagagatcatgtattatcatgacataaatatgcataattgtactatttctcccagtaatatttagcacccaggccccgaggcagagcaagtggagagtgggtgatgcagggctgggggtgtgtatggaggcaccacagaaggtcaacaggcagcgggctgaaggcagggactggactacatgcatcaagtccaggctgcacgaggaaggatgagaaggcagatgagcacggaaatggactgggggaaatgaagaggcaagggaatagaagtctcagtgggtgccatgaccctgtttaagtgattgagaaaatgaacaagatgaaaaggttaatggctgtggtcagaaagtgaaatatgtgaattcaggatttcgaaggtagggtgggtgatgactggcccccagatgcggccatggtgaagtggggcaaaggtgcaggtgcatggtgaggggaaggaggaaatgggaggtgatgatgttggccccacacggacaccacggttgtgcaggaagatggcaggagctgggcaccagggtgggagccacctggagtcaggaagagtgaagagaaaggatgaagaggctccctctcctgtgtctctcctccccaggagaagaacaagaaacaatccgaaagtaataacaccaatgtgcctttacaaagtgtgagtgggtgttgtgtgctgtcacgtgtgtagtaggctcctctgtggatggctagagggactggacatggccactggatcccacttgcaagagcagaggaaaagagtggtcgtgaggaagtaaagccccccaaaatccaggggttgctgcagctttgggtgtggagcgtgccctctgaggaaaggctgctctgggggagattgcccaggaaacggggctcagaggccacgaaagcagctgttaggggcttctgggagatgtgtgctcctaggattagggagttgactctaaggatgaccttagaggttaacagggatgagaaaggggtcaccaaggggtctaccaggggaatgggagaggctgtattgatagaacagcttctgctgcaggttccaaacaagaaatgtgggagaatggttgaaatcagccccgggggcaccttcccgtgcatgcgtgcagctccttcaacattcagtcgaccttcagtgcctcctgtgagccaggcactgggctagtctctgggggtggagagatgagtcaggcaaatgccagccctcagagggctcacagggcagaaggtgagagatgagtgagcagaaaatgaccacagcgcgtgtggggcccagtggagggaaggaggggattcaggagcacaggagagtcaacaggggaaacttctccgaggagaatctgatcctcctcccatctggccaccttctgaagccctctctccccatccaagtgagaaaggacaggcgtatgaccagattggtgtatgaagatgctgaattacgttctcattgtttcaaactagtaaaccatagattttatgtagtaacttctacaaactgcattacaaacactccattctttgttgccctgggtagaagtttattttagtgagcccaagtttgaggaaccttatatggtatgagtacaattaccattttaatagtaagaaatcccccttcccctgtgtaccaaccagaaggtgtttttttcctaatttaaacaaacagatgcagacgtgggctgtccagctcctggcgggatgacatacctcatgcatccagtgggtttgatgatgaggcagacatttcacttaagtgcctgatcatcagattgagtcctgctgggaggaagtgtgaaggaagtaatttcaaaccacagtttctctgtggcttttacaatgtggatatgagaaccaaaatcactacttcttaaccccagagcaggactgattttgaattggtatgcaggcggttccttctgcaggcttcgggctgtgagaagtccctaacagagcaaatctggggacaagggctcaggaaaggttggccacggccccctaggaatgggggctctgcaagatccctggccttagaggctgtgagagggaacaggggtccatccccaagtaagggacacggtctttgaggaaatcccaggccagggcctgaagggcactgtcaggaacacaggctgtttcagtctgttgagattcaccggggcgctgctcactgtgagcacggactcctcaggccaatgtggcagaagagcccacctttgaaagcgagcgggtgggggtggcggggctggtgctggtgcgtgcttctgcacagccacctgggaaggtatgccgctggttgacccaggcagaggttttctttcatggcaaacctgcagtactgcattctcagcagggaggattaatggtaaaagaccaggcatggagcccccttccctctccctcgaagcaagctctgtggtctctcaatcatctttaaaacaccttcttcccgggagcctcctacattctcctggcttccctcccacccccaccctcagctcctggggcctcagcagccccacccccaagcctctaatcttcccagggaagggaacaagaagaaccacattttaaacgaaatttatttttctttcctcaggctcccagttcacatttctccctcaggagtctagggaagcttctgtctggtatcggcctcctcttcacctgggcccccgccctcctcaggtgtaccagaagccagcacactcccccttcccccccagagccacagcagccctgtctcctgggtggtcttgtgtgccaagcctgggcaacatcactcccagcttttcttgttttgccccttctccccagcaagatatttgtatgtaaggtcaggtgagtgagttaaagaataacgaagagataaacagtcaaatggagtcctgactgtcaggtcaagacaacagttatttactgaatgcctcatgtcattcaacagacatttattgagactctgattggatgtcagtctttaatgctgggtgtcagagagaggtgacttcaagggcttgcatctgtgcacccagcattgctaggtacaatgaggagtataataaaagcaggagccatagcccccaactctcaagagatctcccatgtgtgtatgtctgcatatgcgtgcgtgtgcatgtgtgcgcatgtgtgcatgtgtgtgtgcatgtgtgtgcatgcgtgtgtgtgtgcgtgtgttggggatggtgttggtggagtgagagtgtacaaggctgtgtatgaaggggtaattgggaaaagaacaatggagctggcacccagggacaggaggaaaagcaggagggctgggtttggaagacagccggatttatgtttttgaagagggaagactagaatataagggagcagcccttctcagagccctcctcctcccttcgggccctgtgtccagctttccccaaagtccttggatctttcctatgcaaaggggagtgacagtgggcaccactctcagggaacccattactgtgagagaagccactgtgccactgtgtggtcgaacttcaagaccggcttcccctgccccagctgcatggacaggcctgtggggttggcgcaagacccttccagaggaaactagctgcaacataaatccggatatggtgctgttcagggaaaggcacaacctggggatgagaagggtggctgtccagcacacaggggcaggcctcttggccactgggggaggggagaatttggagaggaagaggatgggatgccgtggaattgggaccaggaaagaatggggacatgtgatggttaaagctagttagagaagaactgggagataaacagtcacccatgcccctgaagcactcggggtgaagagattggcattttcacgcaccccagtgctttccctttgtgttgaagtcccttcgtagacatccaggcccataaggctcttctctggccagagcctcatgaactatagcactagcagggttgaggccaagcattggccctggaagccagccgaggaggagggtgcttgtgtgaatctcccaggaggggtaagaattatattaattcgatcataataagcatttattgagtgctgttttgaggcctgggagctaagcacttcacattccttaccccgcatcaacaatcctatgaggtagatgtggaaaatgcagacacggggacaggctcaatcacttgccccaaggtcaccttaactgttaggtgttctttatgcctccttataaagaaaccctgcttcccacaggtgttgagaggagctggagggagcttgactagggctcatcaggcaagccccggcatgtgcctggctctcctctttctacctggagcttttcctgcccttaatggccccaactcatttctcttagtccatgtcagtgccctgagcatctcagcccaagctgagatgatagaaacacccagaggggtcctctaccctgtgacagctgcggtgtgggaagagcacgtgtctcctccaatcctagaccagagtttctcagcctcagcatcactgacacttggggctagataatcctttgtgtgggggagggaggagtgtcttgggccttgcaggatgtttagcagcatctctggcctctacccaccagcacctccccagttgtgacacccagaaatgtctttagatcttgccaaatatttccaggaggatgaaattcccctgtttcagttccccagccccacctcaatgagaagcactgtcctagaccaaccccacaaagcatctgacacccccatccagccctggctaactttttccaccttcttactaaattgggcccagctgcttcagcagtcaatgtgttgggggcagcccactggcaagagcctcacctctaggggctcccagagaccccaagaacagaaccttcctctgagagttgagttacaagtgtttccaatcgactctggctgttttcctttttttgacccatttccccttcaacaccctgttctttctcttattcatatgtag

SEQ ID NO: 11 - RNA, 4023 nt, Homo sapiens
  source 1..4023: mol_type = other RNA
gtagggggatgtcactggccagtggtccctggaggggagggaagcacccagcctgagaaaggcaagaaatatattggcttttttcttctttcttccttgtgttcacattcagaatccatcacttaatgccttgtatttagaaaaaaaccgggggatcacttgagatcgtgatcattttcaacataggattcgaagctgtacacatcctggtgaccttaaaacatctcaggtttttataactggaaggaaccttagagatcatggggcacaaccttctctttatagatgaggaaacagaaatctattcatttattactcaaatatttagggacagttgtaggtactagaacacagtgtgaaccagacaggcaaaaccccaggccagggagcttccattccagtggggccacaggcgatgctcaggtaagcagagactccgctgtgtgacttctggctgtgatgggtgctgcaaggaaaatccggtagagtcgagggttagagagggacggaggggcaggtttaagggggatgctcaggaaggccttcctgaggaggtggtatttgagcagagttgtctgtcagccacacagtaagtgagaggggagttccgggcttggaagctgccagcacagtgctggcaagtgctggggtggcgtcccgaggctacagaacctgagatgctgcagaagagcccacttctgctttcctggaccacttccttctcagcaccaggcaaactccttcttctatcccctggcacatttctgacctgtgtatacgcccccaatttatctaacccctttaaataatctcctctatttatgcagagcattcttaccactaactcacgacttgcacatcccttagctcccttactcctcacaacaatcctgagatgggtcagagaaggaggcttgcgcgtctggtgatggggtgatttgtgcacagttacagggctagaaattgtcagagccagatggaatccaggtcctctcaatcctaatccagtgtttcttacttcagtcctgtggctctcaaagcccagagaccagcagcatcagcgatgcctgggagcttgttaggaatgcaaattatcagggcccactccaggtgaactgggtccaaagccctgggataaggcctagcaatctgtgcttcacaagccctccaggtgattccgcaggctcaggtgtgagagctgcagctgtcctctgggccttctgggctccccgcccagcttcttcagtgtgatgaacacagcgagaatgctagatctgcagcagctgatatcccagacaccctcccgactccctcctggctgggtctgatcctcctccagactccaggagagaacgagacataaacagaacttcagagcctgtgttaaccctgagatcaaggtctgcacagggtgctgtctgagtccagaggagtgagggaccccaccccacctggtcagcaccagctcctggaagcaggttctcacactggttccctgcacaatgaaggagctcatacctgcttttctggcttctcagaccctgaggttttcaccgaaactagacaaggggaacctagggtcagcctggaggcagggtgagcttggcgcctgcagtgcccaggccctgggtggtgcggctccggccaggccctgtttagcttcctctcccacccccacagagggggtgctgtcggcaccgattgctcattttcccctttgctttctcttcagctcgtaaaactcaagtcctgacaatgccttgatgacttccagttggtaataaaagggagatgaagataaggacaggaatttcggggaaatttctttccagttccttactaatgtgacatttagatctctagtactgtgcttctggcatcagtgccaaggcctttcatgttggagaatggaggccggggtcaccaggttgtgcctttatttcatgttgctggctctgatgagctgatgctctgctgattagcaaacgctgagccatctgcgcttcgcagaggcacgttccagccaacccggccctccctgcccacttcccaggatgctttgccttgtgggctcacctgtcttctagctcctgatctgtatctccacctccatccagttccggggctccttatcagcactgttcccagaactgtccatcacgatggcaacgttctctctgggcgctgtccaacatgggagctcgcctctgtgttgtcactcatgctcattgaacatggatttgtgtcctttaccatcaggactggatacccctcctggtcctttctgcctggggtcttagcacagctcagaaggaacctcaccattccctctctccatctagggaattagaagatgacaggggcacagttctctggctcacccccagcccagtaaactcctggacatgcttcaaggcccagctcagatgttgcctcctcagtgaaataatttataaacccacccttctttgtcctgccttctccctcttccctactcactggagagttaacaggtgatggttaagctctgggttcaaatctcacaaggccacacacttagctatgtgacttcaggcaagttaattaaccactctgtgcctctcgtttcctcatttgtaaaatggaaatagtaaaagtgcctaccagcatggcagttgaagttaaaagaaataatatatgtgaacacttggaagggcgcctgacacatagtaaactctcagtaaatactagctgcttttagtggctattcttaacacaccctcttcagtgctctggtttcactatgttttatgggtccctgagatcgaaagtgtccacaccgactcatggtcagctgtaacctgtgcctcgtgtggggaccaggctgccatgtgtagtctggacagtgtaggaggtggcagagctcaggcctgttctgccctccagcccagagagccacgtcgttagatgtcatgggagactgtggtgccccgggaatctcacgaatttgcccacggtactcagtgtctgtccaatgctatgggagtccaggactctaggagccagttaaggtgctgggtggccacaggtccctggccaaggtccaggcctctcccctgccacctgatcctcgagaggccatcacgagggttgtacttcaagaaccactatccttgagctacctaggagctgcagaatgtgcactctgcagggcttagggcctgcagacaagatagatgcagggtgtctagttaaattcgaacttcagataaacaacaaataattttttcaaataattgtgttctattcggtccctatttgggacatatttgtactaaaaagtattcatttatctgaaattcagattcgactgggcatctggtgcttttgtttgctaaatccaagagcaaatttgttctagctacttctcaaccccaccttcagagaggaagccttgatggtactgtaacatcatgctgtaagaaggggatcccttgaattgtaaatggcactctgataagatgaggtatggggattgtattggtttcctgttgctgctgtcataaattaccacaaacttagtggcttcaaacaacacagatgcattatcttacagttctggaggtcacaagtctgaaagttagggcatcagcaggactgcattccttactgcggagttctagagaaaaatccattttcctgcctccttcagcctccagagacacgccacattctttggctagtggtctgcttccatctccaaggccagtgggggcttatcaagtctttctcacatcacatgactctgtttcttctgcctccctcttctacatttaagggacccttgtgattacacaggggcccacctagaaaagccaaaataatctccttattttaaaatcagctaatcagtggctttaatcccatctgcgatcttaattcctgtcgccatgtaacacaaggtattcccaggttctgtgggttaggacgtgggtgtctttcctaccacagggcagtttctagtgttgcctcttctccctgcag

SEQ ID NO: 12 - RNA, 15352 nt, Homo sapiens
  source 1..15352: mol_type = other RNA
gtaagggaggggtttggctgctcgccaattgcaaggtgattcctggggtagcagagcctcacgaattgaccttggggagggcgtgagcctggtgttctggacaatccttgcaaaagctccaggctcccagggctcaaaaaatcacaactgatagtatttctagaacagtggcccagggacccagaagtcactatgaggttcaccattaggtatgtggctgtggcatgtttgtgtccactctaaatgtggggataatcccctttacctcctctaacagagtggtaaaggaaggaggaggcctggtttgactccctgacctgctatttcctagccaggtgatcatggtaagatattgaaccttttctggtcccagtactcatctataaaacaaatataatactttacagagtggtaggaattatacaagaaaagtatacgcaaaacatttcataaattttaataaatgatggccccatgcttcttcctctggaaatggtctcaacctcaatggttggtgtttctagagagaaaaaacgacagagaaagtttcatagtctcaaaaatttggaaagccctgatctagctcaaccctttgttctagaactgcatcccagacagactgcttgggacctgaaaatatctcctcctttgctagaaggataagatgagaaggaattagataaaggaggtgtagagcagaggttttcacactgcaaagtgcataaaaaccatcagagggccgggcgcagtggctcacgcctgtaatcccagcactttgggaggccgaggcgggcggatcatgaggtcaggagatagagaccatcctggctaacacggtgaaaccccgtctctactaaaaaacacacacacacaaaaattagccaggtgtggtggcgggcgcctgtaatcccagctactgaggaggctgaggccggagaatggcgtgaacccgggaggcggagcttgcagtgagccgagattgcgccactgcactccagcctgggtgacagagcaagactccgtctcaataaaaacaaacaaacaaacaaaccaaaaaaacccatcagagaagttggtaaaagatgcaagtgctaaatccccacccccaatcactgtgattcagaagaaccaggccaggcccagaatctatcctgttaccttaggcgattctgatgaagaccattgtaggccacactttcagaaacactcaaaattagaatccttcagagaaggtggcatatataatatttctagcatggaattatgttttttttcttttgcctacattttaatttctagaactgtgttgtagggaatgtcagtcactaagaacttgattgaggaactgtgttttgtctgtttcatgactgctctctcaagtcccaggaaactcactttcagcttgtcttaaaaagcaagctgaaggcttttaaaaatgaagcaacatgaaataagacaccgcagtttctggcacggtccacgcttaatccccttcaatgtgtgactttccgtggaaagttactctacgattttcccagctcgtcagggtggggccccagagtgagtatgaagggtcagagcctagggatgccaccatcagtgagagcccaggaccccagaaaaggtctcttggctcaccacactgtaggaaaaataaaaagcaatgtagtccaaatgtctctatccaaagtttcaaaaagaacttgattttagacacgctccttgacttgttttcagaatcagacagaagagtgaggcaacaaaggtcccttattccaggcagctgaataccagcacagccaggagtccagtgctggtgtttgcagagccaccagaggctccctctcaggtgtccagggcccgcatgctttgtagaatgggcagaatgagcaatgtctgtgcacctgggctttgcaggcagggcctgggtacccaggttcgtgcaatcctctcgtcaccatgaagggagcagcatcattcttcccttcttgaagcaccttggccaccagtataggtaaatttacctcccaggacatgaccattgattctgggatgtcaatgccagagatagtagggtaaatcggcacctgggtaaaactttccattggagactagaaccaaaactcaggacactggcttccaaatgtttctttatcagacaagaaagaccaagtctttccttacgtcttcacatgctgccttggcaaatgctagcattcacaaaccctgggctaccttgacctgtcacccttgcagacctcagacgggtcctgggggcttgctttctcggtttctgtatgcaggcactcaaacctgcatcaggcacctgtgaagggccgggcactgtgctgaggccaaggctccaaatgtgaaccttccaccctcactgaactcacagccagaccagagacaagcaaacaggacatttcacagcagtgcagcctagaaagggccaacaccagcagcatttgtccccccgagcggtagcttttagaagcttccccagtgattcaatgtgtcctacaaatgcctggcccccactcccagagattctgagtcagctggcctagggtgcagccttgacttcactgtgttaaaaagcttcccagataagtccaatgtccggccaagattgagaatcactgacctagagtttaatttaccacctcagtctctatagaccacgcataataatagtaccccacacacctctgagggtccaaagaactttcatttgatcacccatgagaccaccgtggtgtggagatgctttctctctcctgttctcttaacaaagctggtgagcgacagagcctgcagtggaccgggagatggcccagaggagaaagctctgccgtagtcggcctcagttaaccacggagcaccacccctacctgctctcctctcactcctgcttccgtctcggtggagaaagatccaaccgaagcaggacacatctagtcttctggtgcctttaaaatgtacttttccatttgacaaatggattacactaaaaacaaaaatttacaaaaaaaaaaaaaaaacctgaaagaaattgcaggcattaaaatgggactttgcctttattgctcctgggcccatcctatttgggtttttagaaaaacaagcctgaggcaggcccagaaaggctcagggcagaccctccgatcctctgaaaggagcatcaggcaggcaggggttgctccggggccagggaaggggccccgctgggacgcggctgttattgcagctggttggcgcgcagccatgcttagctgcagtgcgggaatgctgggccttctgttctgggctgtttctcatacgcacgtaggccagtgtataaataaggttttattaaatgccaaatgagttctcattaacaaagaaagagggaaaatctcagtaaaccaccgtgacggcatctacccactttgagtcaggagctgggggtgtgagtgcaacctccgagacaagggaacctgtggagcccagagaatcggaggggggcgctggggttagcaccgactgagaccagctgtgttttctctcggttccttggagatcagaagtgagtgttgtcatcttcaaacaatccaaaggcagtacccatggccttactacatccctcccacaccatcccacccatccccgcgcgtacactcacacgctcatttgcacactatcgcacacgctcacttgcgtgcgcacacacagattggtgacctaggtggactgggagagaaataagagccaaatgactggattttctccaaggaaatttattaatagcccctcttggtttcacctgaaggagcttgtcttcacctgcggcctttgcaggcttaacgcccccagcttgaaacccagaagctcagacttgggcccaaggtattattagtgccaacactacctgaaatgtttcgcacctcataaaaatggtgtgtcagtttcgggtgagaggttgggacgcttcccatctgatttggcccaaggcatgcatgcccctccttctccttcccctcctcctccccctcttccccctaccatccttcctgttttctctccaactctggtgcacagctttgaaatcttgctgagaagcaaatctgtcccttctgctttgaatgtttatttgtggaagttcggcaggggaaccgaggcgggtgccaagacctgccatgctgctgggaagtctgagtctccctcccttccccctcctaaatgcttgttgatagagaaaagtcagcctcctcggcatttgggctcacggttttcctttgaaaatgcttccagtgtggcatgattcagctttcttttctgtcccccaaccactgctctgttgtcatttttacttttctgattgcattttatccgtgtctctttgactacggggtggctggacgttgagttccaggaagaaaagggcccaatcttggggttctgactacatgcgcccatcaatgtcctgtttcattcttggctctggctccctgaattcctgagtcactggggagaagcgtgggtggaccgccccctacccagtgagagttgccacagttgctgctctcctgggtcattggttgcagattgttaaacttcacctatgcatttcaactttcgggtggatattgctacgtcaagtgtctgggaaagcccccacagctacaggattttacagtgaggtcccactaatgacttgatgtcatgacttcctcattctttccaatttctcccacttctccataagggttttgggaaggggagaagagaaaggagtgattcctgagtgccagtaccagggaacagcagggctgttgggaggaaacaaaactaaatcaggaaggtttttgttgttgtttttggggggttttatgaaaatattcaagccacagcaaatatatttgatttatagcattagtattttttctgcctgcatctacaaaaatctttacctattaccatcaaaatatcctctgggtgaatggatttcaacaaagaagaaataaaaatgaaatagaagagaggccccttcgtgcacattgagcctactggctggattgtcacttgcctgccttgatgtcttttcagctccaggcaggcagtaggccagggcttattttcatgacagatcagatgttcttttatggatttacaaagaaagaaatactgagaagtcaaaactgaagtcacttaagacaagagcaggcccctgggaaggctgccattgaggataatgagtcctggggtcctggcctttgttcagtaaatacgcactaggcgcctacaatgtgtgcaccaatgtgtgaggcgtcaggttctctccagggtcagttggttttaagaaaggttttggcttctgatatgttttatctctacagaacagtagctcttaacctttcttatgggttaggattaccttcgagaatctgactacagctctagacctgttccctaaagaaaactaagttcacagggacacacaggatggggctcatggagcagctgaagccagaccccaggttaatagcctttacattaaaatgtttttctacctaccactaatatgcattctttagtaagcggtctcaatatacaccgattcttccttaactctgtttatgaagtattcagcatcctccctgcccccttcagcatcctccctgcccctgagcacaggatccaatggcgtgaggaccacaggcctgggcagctgctggggcatacaggcatctcttagtggctgagagactgggccctggctctatgttggctcctaacttgctgccatttaaaggaaatcttagcctcccatccgtaaaatcgagaaaataagacttgtcctacacagctcatgaaatagtaatgaaattcacattagagaagagatggaaaaacactttgaacaaaaagcattttgctcttataaaagcacagcctcttttgagaggccctttgctccccatttctccttcttcagacccccccagactaggagaaggtctgtctcatggagtgaccttttggctgcctctagattccaagctcagttttgctttcattaaccacagatactgggacggacagaaaaagacctagtttctgttgagccaaagagtctcataacttgtctgttcacatacccaagagcccaccctctagttgagacactcagttccctctcattctgggagactgcatgtctctgtgacctcctggtagagaccgtttgacatgtcccccaaccccccagtgattgagtctgaattctccactgatgacgcatttcctagcactcagggtgtcccctcctggttgccccctcaccactgaagcccgcttcctcccttttcatttgatgcttaacaactgtcagtttgcaagaaacatgcttcaaatccacattctcccagttgcctagcaacaacttccctcccggataaatgtgggtttcctgtagctcagcccaggactgaacacagcagcacacacttctgtccactgcttcaactgcttttcacctctggtctgcatgccttcaagactgcagctcatccctcccttcagaaccttccatagcctgcagaggccatgtctgccccaaaaagacacattgaacctgaggctacttatttacccttgtgttaggtatatcctcaacttagaaattaatactgtttccagattgtcttctttgaatcacagaaagtaaaacaacaaaacattcaatgcttaagacatttcatgtgcggttgggtgacatctgtttgatgaacacatttgatccaaagcatcagaaatactatgccaacaagactttttaggaggtgataaacatgtctgttctaccttaagaaaaaaatattacacagtcccaagggagagacatggttttgatcccagacaacccaagcagagacctcttagggccggaatcatcttggctgctgcctaggaccttatatcaatttcttaagcacaggatcaaggcctaaaggccccttagactgacctcagttagtagaggcagatcccttcacagccttatcttccttagaggtctagtctgaccttgaacttcggctggcagtgctgtcagttgtgatgtgtgacatggaagagttatttgttacttggaaaattaagagaacttatttggcataggaaattgtgtgtgtgtgtgtgtgtgtgtgtgtgtgtgtgtgtgtgtgtgtgtgagatgatgtttgccattttgatctgtgacttttttttccagaaatagtttctcagttccattccaactaaacttacagtctcttccggttctttgacagaaacaattcatgtgaatttgaacagataatagggaagggggaaccaaaagaagaggagagccctgggaaagttattttataatttatggcaacctcagtcaggcaactgtgaacaggtacatatggagggctccctcgggactaggcagtattcagagatgtaaggtgtgaggaccggaccctcatcatttaccattcccactaaaaagagctgggaaggaaattgtagctgtagcaccaggcacgtaactggagcttagtaactatttggtgaaggaatattattaaattattaacaagatggaaaaaagggtattaaccacacaaaaatacatctcaagctattgtttctctgttccctttcccccaaattcctagtcttgctcttatctggctgtctctctagtcactctttcttgctgactctcttcacgttcctttctccacctggaattcctgggccctccccttttactgacagacactgtcctcactctcacagtcatcagtttgtctctttacaaacctcagctcaagtgtcacttccccgtccccaggtgaaactgactgctccctccctgtaagtcaccatgatgactgctatatatagccctcatggaacctaaaacctcaacagacacagtctctttcctactctgttatagtttatttactcattaattaccacaacacgtattattgagcacctactgtgtaccatgcccagaagataaaagacaaacaaaataaaacctattcctatgcttaatgagtttacagtctagtggagagatagatacattaaaaaataacagcaaaccaaaataaaagtggtaaataaatgcactgagaaagacaggaatagctaggaggggcacctaatccctagggaaggaaagctggaagagcatggtgatgggggaagaaggctttctggagaaggtgaggtagtttgaaatgagttgactctggccagtaggggtagagtgagaatggggtgagacagggtgggttggtcattttgatccattagtcctcaaagtgataggactagtggctaaggactgcaggctttacagaagcctacaaaactatttgagatttgaagtttttttttttttttaattggctccaaaagaaaatgaaaaaactttagaattataatgaatgaatattaaatgaatatttaaggaaggtaattttattcaacttcattgttaaatttagttaaaacaagcccttgagtttcattcaacactgttttatcataccgttgatgagagaaaacaaaactgattcctggccagggccactgtcagcgtggggtttgcacatctttcccatgtctgcttgggttagctccaggtactcctgtttcccccacatccccaagatgtgcccattagtggaaacggtgtgtctgcatgattccaacgtgagtgagtgtgggtgtgggagtgagtgcccctgccatgggagggcatcctgtccaggttagattcctaccttgtgccctgagctgctgggatggaatccagccacccatgactctgaactgaaataattgggtgaataattatcttactttttaattaatctttgaaaatgtatgtatagttcacatgtatttcaatatttaatattagaagtattttagtctttattttgaagtttggtgatttattgtaaccagaaacaagctatagaaacttaattttgggccaagtgcagtggctcacacctataatcccagcattttgggaggccgaggcagacgcatcacttgaggtccggagttcaagatcagcctggccaacatggtaaaaccctgtctctactaaaaaatacaaaaattagccagatgtggtgggcacctgtagtcccagctacttgggtggctgaagcaggagaatcacttgaacccgggaggcggagcagtgagcagagatcgtgccactgcactcccacctaggcgacagtgtgacactccatctcaaaaaaaaaaaaaaatagaaaagaaagaaacttaattctggtttatatcaattagcctgtggtaaaattggtttcattatagccatttcacttagttgaagtttccaataacctgtggatgaattaagtgaggatttactatattcataaaatcttaaattccaaagcctgtttgcagttcaggtttttccactttacaaacacttctaagtattcacaatgattgcttaaaattcataccagataaatcattaaataagttgttcaaagtcaaataatttcataagtaaaaattaggagcttttagaaaactatacctacatagacctagacctatagatagacagagatctgaatagatatggacacagatgctttccaaagtgttcatgtgatgtgtggtggagtttcaagaccagagtgtgcctggggcctgcagaagtaaaggagaggggatggagagaagattgtccacatggccatgggcaatctcccacccacactcaagtgaggaagacaggaaacaaattcagaaagaagagaaaataatcaaaactgatgggagcttgtgactgatttacttatgcgcagcctccctggagacatgagtgtggctgttccttaggttgtgcctctgggctcctaccccctcttagatgccttcctattatctaggacctggttgctttttgtctgcatagcttctttggattccagtctttgatgccagcttcctcctaaagtagcctttcagatgtcccttggttaccctctgctatctaagggctcatcctaccccacactcattcccagcaccaatttctggatctccaggctggagatttagacaatgggatgggaagaacccatgatgggtcccagacagaaagtggtgccagccacagaaagggcacacaggcacagaagttggtttggggtaagacgatgtggtcagttcagaacacgctggatctaggcagatgcccagcagacagttggatatgtaagtctgaagctctggggagaggtctaggttggaggtacagatttagaagtcatcaacaaaaaggtagcagattaaatgataaaggaaatgagactatccggggagtgtgcagagtgagaggagcaagggaggcccttgggaacctcagcacttcaggggaaggtagaggtacagttgctggtgggaaaggcagagaagtagcaaagcaaaccaggcaaaagcagtgtcacagacgaccagggaggaaaaggacatgatcaaaatgttgagaaaagcagagaggtttgaaaatacaagaagcaaaaatgtccactagacttaaaaaccaggagaaaactggggggttcttgataaagcatcttagtaggatggtgagggtagaagccagggaagtgttggtgaggaagtgaagtcactgattacggactatgcttaaaagaatgtgggaatgaagggtggaagagagaaattagactgtagctagggagacataagcgatcagaggtagattctttctctcctgtgggagaatcttgcacgtatacacagcatgacgacagtgatggaagggctggcgaagcctcagggagactcttggaggtaaaccccatgaagggaggactttgtttcattcactgccgtgtccccagcacctggcacaatagcagacactcaatacatatttgtcaaatgtgggattttatcatttagaaactgcacctggctgtgagtaacaaaagtcagagaaaccgtgggtttcatttttctccccaggcagagtctggagctgggtcctccaagaggggtttggagcaccacaggtttcctcaagacccccaggctgccctgtgtttccctccttcatccccagcatatgcctgtcatctggtgacctccaaacacctgtgctgcctcctccagcacatccatgttgcaggcagggaccaggcaaagggcagaggggcctacttcaaaagaccatttccagaaaccccatcctatgacttctcctggtgtcttggttaccattgtgccataggctcaccctgtatgcatgggaggctgggccaggcattatgacttttagcaatattgcatagataagcatcaatctttgtcactgtgacgaagcctagtcactcagtgctaggcaaggttaatggaatgggttggtgtgtgcattattcttgaggtctttcttatgcttcatgttatacatttattaggacgtttaggcaacagggggataaaaatgaagaggagatgcatgctatgatctgaatgtttgcatcctccccaaaattcatatgttgaaatcttcatccccaagatgatggcattaggaggtggggcctttcggaggcaattaggtcatgactgggattagtgcccttgtaaaaccccagaaagccagcttgccgcttccaccatatgaagacacagagagaagatgccatctacgaatcaggaaatgagcccgcaccatgcaataaacctgctggagccttgatcttagacttcccagctgccagatctgtgggaaatagatttctgttgtttacccagcttatggtattttgttgtagcagccagagtgaactaagacagtgctgatctcgtattcttggagggaacccttagtctttagggaaagcaaagccaccatttggggcagggtgttctccaagtgctgccacatatgctgatgtggttaaactgcaaactatggtaaaaatgtggaggtctgtggaattgtcaatcaggaaaaagatataaaaagaagttaaagtcttcgtgcttctggaaggatatgtgccaaattgttaacattgattatccttgggtagagatgtggggaagtttgcagagacagttttgccttgtactttatataagtaaacagctactacttcgttgtcttaaaaaaaaaaaacagcctatgtgctcttcatgtgactcagaactacctaggcaatacgattaattgaattagtaaaattgagtgattatgaattttcaggaagtcattaatttaccacttctttattacatccacttctaacaggacttcaatataggggaatttgacttcaagataaaaagaccaaatttatttacccttttaaaaaaagacaacttaaaagcagacttgtcttacagaaccttccttagttggacatcgatgagtgtacagaaaatgcaatggataaaaagcttggtgatacaaagataaaaagtggggtcctgtccttaatgaacataccatttcatggagtatcaggtgtataaacaattataatcaatctgcttgttattctgataagatcatttactcacacatcaaatactgagtgcccaccacatgcccagcatacctagaagtcatccagtatgatttctgtctacatggagcatagagtcttacaggggagatagatgacaagtaaacaccagaataattaccaatggtgaagagcacaaggaaggaaacagaactcctaaagagagcgtggctgggcaggggtgagcaagaggcatagaaaaaggggcatctaaatctacttgggaggaagctgtttctcacataggtcatcatgttaggaatgagacttgagggatgagtagaagtttgccaggcaaagaaggaatgggggggaatagagagcagagctaggggcaggagacagctgacgtgtgagcagacataaaaagaagtccactgtggcagcagagaagcaggagagaaggcaagtgagggagccaggcaccagctcacagaggtcatgtgtgtcaaaacgtagtaatggccttctcttctggagacagtagggagccatggaagatgtttgagcagggaaagcgacatgactggattggcctgttgggtaactcagaccacaatgcattggaagggagggggctagaggcaaggggactggcaagaaggccagtcctttttctatgcctattttgatgaaatattctagaagggaagtgaacaaaggtagtcctagagaggaagaacaaaacagataggatacttccttagtatttgctcattcgacaatttatttttgcatatacactaaaaccttttttattattaaaacgttttattgtaggaaaaaagtatgaaagtagagtgaataataaaatgagctcccatggatctatcacccagcttcaactattatcaatatttggctgttcttgttttaactgttctccacctttttttcctgaagtttttttgaagcaaatcacagacaacatatcatttcaccatatgtacttccctctgtatctctaacatgtaagaacttgttttaacaaaatcaccatgctatgatcatacccaacaaaatttatcataatgtcttaataatacctaatacccatttcatgtccactttcccccaattgctacagctggtttgttcagatcagaatcaaaatccacctgtggccattttactgctatgtctctcaggtctcttttcatctctaataatctcaggggagacaggagggaggacgggcaggacttggggctaacttgcttatcgacacacagttttgcctacttgcttcctcccttcacacccactcttcttctcagccccacccttgtatggaaaaaacagaaattaaagtgctttgcccagcacccactgaagctatttcgaaggagtttgaagagtactcccggcaagacaaatgcctcggtccagtgctcaggtcaaagaggggagacgcttctcagtgatgtggtgtcaatagcagcttagttgttctttcctctggaaaattctacccatctgctttgtaactcccatacctaacaaggccttttatttcacaattagaaaataagcctgaaatatgaatgctgcctgagtgtacctacatttattctagagtttcagggtcaaaaagaatacaaggacctctgcatctacagccaagaggagaggggcaaagacacacagctacaaatgagaacctggctggtcaaagcctaactccacctgtttgtcagcactgatgcaagttaggtcagcccaatgatcatttaggagaactgtgctggcaaataaaaagcagaggcttttggtccccagatacttggatgagaattacaagtccagctggttaaaaggcacatgcccagtgctcacttcacacctactcaggaagcacacttgagttggaaaaccactgtctttacacttagaactcagtcctacatgactcctctaggatcagtgattccatcagttttgaaacatgaagcatgaagtcaaacaggacatgaccttggtttccagaaaaccagatgttcacatcagtctctggagcttggaggcagcacacctggggacttccacatcccctgccgaggtggcaaaagcaggagcagtggtgagttcacatgggctggggtttcctgaacactgctggcaattggagaatctgcaagggaacttctccgactcctaccagcagctgctttaaaataaaggtgatgtagctggtcaaatcctccatgagagagcagtgttgaatggaggaagagacacaacctgtctgaaaatggcacaaaggaagaaagatgtaaacaatgacgagaagactgcagtgtctacaaagctccgaggtgaacagatgggcaccccaggcccgcagcacttccttcagtctctgccagctgcactctgttttccttcctccaggaatcttgtttggtgtcactaaaacagcaattagaatcactttgaaatagtgatagtatttaatataactatgaaactatctgtgattgacaagtgcagcaaggagtcttggaatgagagcctttattttttcaattaaataaaagagttttttgtttctaaaagtaatcttgcagaaaagatcctgcgatcagaaagaaggagggggggagttttcaaacatataggagatcagactgtgcctatgtgtgtatatacctacaaacatatatatatttaaaaaattgttttactgtcaattacag

SEQ ID NO: 13 - RNA, 2633 nt, Homo sapiens
  source 1..2633: mol_type = other RNA
gtgagtagcctgatggtagtaattataatttcattatgtctttccaccatttaccccacttatgtcaaataatttaattgtatttcaaacctgttcaaggaaaagtacatttgatctttccatctagcaatttcaaagcacctgttcacatcccaaattatctgtgctcttaagtaagaggcagaaagaaaggaaccacccttctgatttcacatcaaaaaagaaatgccactggcaataagcaacttgcctggtgtggcataaatcatcagaagacttacagttgaatctaagtcttttcagtactgaggtggttcattattctgttacagtcttaaaattcacataaatatatactgccaataataatagcatacacctttatagcttacaggcactcttcttctaagtgttttacctatgttggcttatttcatcataaagaaaacaatggacttttgtgttgttttgtaaaaagatgcgcacattttaattaacatctgattgcacaagtctcctcccatatagaaatggattcttccacgcaatagataagaggtgctggggatatgatgatgaacacacagatttggtcatgaccctgtgggaaagagagatgggaaaaaaacaattctcttcaagtgtgatgagtgttacgaaagggagggaaaagttgaaacaggtttttttccaaacttttctccctccattattcgcagctgacttgggctccaccaacctggaaaactgcatggttggaatctgtctttataaaacgcatctcaacctgggccgagtatgcacactgatgtgggaaagttagagaagagcccattgtactaatgctcacctgctacagtgggagtctctgttaaacagtcttttcttcatagcattaaaaaaatttatatcactacaataaggttgaaattgatagagaatgtacaaacaatccccaaagtatatcaacactcttagttctgagtagaagttccagaaggcttcttgactgtctagatagcaagtctaatcatttgtgaactaagttaaagcagaaggcccagtttatatgaattggtattacaccatttgacctgagaacagccccttcatctctgagtgctttgactaaatgagcaacataataatagtaataaccccttacaagatgtcataagactcactgttgttgaagcaatttgagattttgactttattgaagcatagatggtgattataggcatgactcactgtgtggattctccctgggctcatcagtttcagagggcaagtgttggcatgtggacaaagagagggatgacacgtaaacatggcttattgcaatggggaaatattttcagtctcactgattgaatcctaatggttttataaattccccagtaccactgaaagcaaagcaagtaatcaggtgtgttttaggaataaaagcagcattattttaatttcgtattttcccctaaagcaaagccaaatggcattatgggagccaagctactggcagctccaccagccttctcctgagttctcggcattacagatctaccctcaaaggatgaggccagcaagcaccacagggtgcccacatggagaagagaaggccaccaacctcctcttagctggcacagaattgaaaaagtgtttttccaggaatggatacttcatctgttctgtatttgctagaattttaaaacgcacacacagacacacacaggcgtgcacacacacacgcacacacacacgagaaaaccacaaaccacacatttcaaggaaatggaagaattcattggtaaaattaagctaataagattattttccaaatataagaaactaaattttagactatttagccaaagaaatttgctctgatcttgcttttctacaacagaatcattccccaatcattttatttccctctttttctccccagtatccccatcttggtgggacaacagaacccaagaactggcttaacagtaaaatattttctgcatttgcccaaggacacattcccaacgaattcaaataaaggagactagaagaagagaggctatactacagtgctctaggggtcactctgtgatttgttgttgttgttgttgttgttttgagacggagtattgctcagtcgcccaggctggagtgcagtggcacgatgtctactcactgtaagctctgccccccaggttcacgccattctcctgcctcagcctcccgaatagctgggagtacaggggcccgccaccatgtccggctaatttttttgtatttttaatagagacggggtttcaccatgttcgccaggatggtctcgatctcctgacctcgtgatccgcccgcctcggcctcccaaagtgctaggattacaggcatgagccactgcgcccggccactctgtgattttctttaaggctcatcctagtattctcctagtccctaagtagatggcagtaggttttgttttttgtttttcgcagctggattaaggattgctgagaatatatggatgttttcttttaaatgtggaagtcaaaccaaacgttggagcattggcctcacagcagattatgactctagctgccttaaaataacctgaagactttgccttgccctag

SEQ ID NO: 14 - RNA, 1016 nt, Homo sapiens
  source 1..1016: mol_type = other RNA
gtaagttttctgagtcctgcttataaattggcctctcatgttggttaagttgatggtttaacacttctaggtgaaaccaaacctggggttgcatctgtcttgtcttgctgagtggccttaggtaaagagacttctcccagaaagtccacttccccttgcagaaagggggcattgcttataagcaattctggacatgaaccacagaaagaactgaggcccacttggaaagggaacagaggggccatttcccactgatgtaattgaactagggctaagttcaagaggaagagaatgatccgcaaggaagcaacccagagttccaggtgaagctcaggtcagaagggccctggcaagtaaacacggctgtgggatgcttttacaaacacaatatcgtgaaaatctatgtgtgtagtactgaattacattccaaatggcaaattcctggcaaatcatcttccccacctttcactatttttttttttttggtcttctatggggtaaaggaggatggggtggggaagaaatgtaactggctgcccctctagttaaaaactgaaaagaggcagcaagggacatgccaaaagtagttggactctaagatagctacacacaacaaagcagctaagcagctaattgaagggaaattactgaggctcaagctgagattccaagcgggggccttgtttggcctctcagtccctttcatctgagaaaggcctcagttcctagcagtaatcagaggcaggcttctcagcctccttctcctaaagcagaataaaccacagggcaagtcgcatcctttgtttctctgatgaggccattactgagagtcactgtggcattttgctactaatgatgagcttgttattggtggggtacagcctattaatttaggttattcatcaaatcctccagcatggagttgaatgagacatgtgatgtggatacactaatgactatattgagttacaagcaatggggagtttctgtaaaatctgtcccttgtctcctggcag

SEQ ID NO: 15 - RNA, 615 nt, Homo sapiens
  source 1..615: mol_type = other RNA
gtaagatcccagctgggcttgccttgtgtaccctggacctcccagaagtgtgtgtgtgtgtgtgtgtgtgtgagagagatgtgccttcctggtagcacatctcatgtttgttttttgctaagtggactcttgcgtttcctcccccatccacagtcatcactggaatgctttgcttcagtgcccctgcctgggccctcccctctctactgcagcctacaatgaggttttctttcccattgcttgaattatatccctaatggaagggttcacaattctctgaatcctggctactcagataaagacagggaggaagggaggaagggtattttctcccagggggtccaaatctagctttaacgagggaggttctgagaaaataatatcatcaatattacatggacttctgagatactaagaaattagattctgtcagcccaggaagttgggagatggtgaattgttctgggaaatagcaatagactgagaaaataaaaacacttccttgaaaagcctttccctaacactaagtgataggggcagaaaagacacaaccaaaagttctctctcacttttctctctgttcgtgtctctgtcttgatctctgtctggttttag

SEQ ID NO: 16 - RNA, 702 nt, Homo sapiens
  source 1..702: mol_type = other RNA
gtaaggggggttggaggatggggaggggaggggaggaggaagcggtgggggcaagaaagttccacttgtttccttttcccaggaaagagttaatcgctattggagttagatcaaaatacaacaagcaggccccaaaggccttcattccaagcagtcaccaagtggggtcactgactttggatgagaaatatgtttcttgaattctgggagaagtctaaaagctgccacaagaccagtggcttcctggagtttcctacttttatgaattcactcaagggcctcaaattcaaagaggcatctccccaaggggccagctctgtaactccaaagatggtggaatgtgtttgtctggtctcattttcagctttgcaaaatgaagacaagagttctatatatcagggacactcaaaagaaaacaaaaatatccataagcaaaagaaagctttttatacaccatattcaatgacccccatctggcccctcctttgcccctacacatcttccctctattctagagacccatggacttggggaaatgggatatagataggtatgtttcatagtggaacaagctcaccagctcttcagggagccttagcatctctatcctcaatcactaaaaattagaaatggctgaagaacaagaccaaagatcctatggaatttctaagcagagcagtgactgtatttcttcttcccaag

SEQ ID NO: 17 - RNA, 14372 nt, Homo sapiens
  source 1..14372: mol_type = other RNA
gtaaggggctgcaagccccacagtgggccccttgaagatagccccatgagtggggccagagctcccttagcaagtcaagtggtcttgaatttaagctttcattttccccactgaagaaacaagaatccctacatcccctgtacagttctcattctctaacagcttatccatacttaaaacttatctatgctgaaaacggtttcctcttcacatctcctacttctcatgctgggcacctcctcctgtagccccctttaagcatctgtgtctgtcctcaaccctcttctgtctgacattgcttgagtggccatctatggccagtgtcccctcaaccccacagtccattgcttgctggacactcctgccctcaagttctacaagcacatcagcctcaacatgtcccctccaaaaactgtatgttctccttgcccatagaacatatccttctcctatatttcctatcctaattaacgtcctcagcatttgcccgaattctcaagtgagggatttcagggtcatccctaattttccttcttcaccctccacacagtagctgtcacttactgagtgttactttatgccaagtactgtgccaactgcttttacacacatatgcttcatttaattctcacagctccatgaggcttgcaccattatcattgccaatttgcagatgagaagccagggcttaaagaggttaaataagatcccacgcatgaccattaagaggagcgaacaggatccagctctgggggtgcctgagttcagagcctgcctttctgatttctcttaccaagctttgtctcctctccctcctaaatatctctcaactctgcctcttgcattccaggctctctgaggactagaggccttgtcatctctgcgccagcccattccaagggcttccttcctggaatccagggtccagcctctgttggcccaggcatttctctacactggcaccagagttacattccgcacacctgcttacgttgctctctcacttaaaatcttaatgactcgacccccaaataacacaggtcccttccaaatctgtcctaccccaccttcccagcccttgctcaactctgctacctggccctttcacgcctacaggcattcccattccatgacctcttgggattctaccctttgcaaatgctgttttcattgcccatttattagagcgcttttggtcacaagctttttgcttaaccaaaaagaaagcatttattggtggacataaataatgaagttcaggaggatccaagagttggaagccaccatgagacccctgtgtccttccacctcacttttctactcgcctctgctcagcttcatctctggccaggccctctcctctgctgatgccctagctgcttacagcccttagcagtcatctatacaccaaaaatccctttcccatagcagaagcaatgctcctagagagtttccctgttggtctggctcctgtacccacccctgtgtactctgattgggaggcctgggtcagctgcccaccatggggccatttctatgagcaggattactgtgaagtggaggaagatgtttccccaaaagaagaaacacaaggtagaaaagtgtatgtccaccaatgcctgaaatgactgtccctttcctcatctgctgagcttctactcattcattctttgagactcagcactcagctcttaaatgtcacttctgctttgatggaggtttagtcattcactcctctgtgcttcctggccctctcttcacacctctctcagacccctctcccagatagattagagttggctgttgacatgtccatctctggctgggcagctaaactggagttatttagaatcagggagcacatgtcagtcattttcaaattctcaacctcatactcccagtaaatgactccatctaagggtggaccactcttgcccatgggccaggtctgggtctgtgtcatctagaactgttggaaggtaggggcttctgtgagcagtaggagagggaataaactcgagggccctcgggagcatgccctcttgtctcagacttgtgagtcctgaggataacaaactagtgaagaaaagcctcgttctatctgtcacctggtgctcttgaggactttctgttgccctggtgccaccacaattttccagagtgtgtgaccctcgctctccaaactctggaagtggcagccgaggctccccagtggcctttcagaaggtgccagtcatgacagcagcaccaaactgcaggcaactactaagcgatcaccaacttgtctgaagataagaatgaccttgaatgcattttataaaacaggattttttttttaatttttagattttctttctttattttaccttaagttctgggatacaagtgcagaatgtgtaggtttgttacataggtatatgtgtgccatggtggtttgctgcacttgtcaacccatcatctaggttttaagccccacatgcattagctatttgtcctaatgctctccctcgcctcgcccctaccccaccccaacaggctccggtgtgtgatgttcccctccctgtgtccatgtgttctcattgttcagcttccacttacaagtgagaacatgtggtgtttagttttctgttcctgtgttagtttgctgaggatgatggcttccagcttcttccatgtccctgcaaaggacatgatctcattcctttttatggctgcatagtattctatggtgtatatgtaccatattttccttatccagcctatcactgatgggcatttggattggttccatgtctttgcaattgtaaacatacatgtgcatgtatttttatagtagaatgatttatattcctttggttatatacccagtaatgggattgcctggtcaaattgtatttctggttctagatccttgaggaatcacactatcttccacaatggttgaactaatttacattcccaccaacagtgtaaaagccttcctatttctcaacagcctcaccagcatctattgtttcttgacattttaataatcaccattctgactggcatgagatgatagatacccatttgtcagatgggtagattacaaaaattttctctcattctgcaggttgcctgttcacgctaatgatagtttcttttgctgtgcagaagctctttagcctaattagatccatttttcaattttggcttttgttgcaattgcttttggtgttttagtcatgaagtctttgcccatgcgtatgtcctgagtggtattgcctaggctttcttctagttttcatgattttagattttacatttaagtctttaatccagcttgagttaatttttgtataaggtgtaaggaagggatccagtttaagttttctacatatggctagccagttttcccaacaccatttattaaatagggaatcctttccccattgcttgtgtgtgtcaggtttggcaaagatcaggtggttgtagatgtgtggtgctatttctgaagcctctgttctgttccattggtctatgtgtctgtttacaaaacagattcttaagcatcaacccagatcgactggctcagaatttccagggaagaggcctggttatctgcatgtttacagacctattagatttgtgggacctgcagttcccttgtacagttagttactcaattaacatctccctcctctcatggtgcctctacctgctaagcccttattcccagccaggcccaccaccatccacccactgctgttataacataagcaggacctgtgcgagggggtgtggacggaggagagaggctctgttgcttcatttgtgcagcatggagttcagtggttctcacaatgtttttgcaaagtatataaagaatactccttgtctacttgacattcgtatcgtgacataaatgtcttgttttccagaaggattattttttccaagcagcttgttcctaatgcagccccaggcaccaaacagatacttaaaatatattaattgcttaaatggttaagaattcagtctctggacccacactgcctgggttcaaattcctattatctgtgcccagtttccaagtctataaaatagggatattaatagcacttacctaataggctcgttatgagaattaaatgagctaattcatgcaaagcactgacatatagtaagcacttaataaatattagctttttaacaaaatacaagccaaaaaacactgcttaggagaggaaatgatgttagtgcctcctgtaaataggcccagcctccaagctggtgctcctctaggaatcacaacgctgcaaatcacatcctccggggccgccaggacttcacgagggcctctgagcagaggggtatgatgggagcagaagcccagcagctgtgatgatgtggtttctgatcttcctgcccttggggtgggggaggaggaaagcaaggggcaatgaacagaaaggagaagatagcggggaggaaatgtgtgaggaagaaacacatcactgtggcttgtcctggatttttctgcttctgttctcgtgttttgggaagtctggaggagacttgaaaatcattcatgtccccaccctgaggatggcttagtagcagagaggccatgaaaactctttgctgatggctctgaaagcaaggatgttgcttcactgggctgctgaaggcctgcctgggggttctgagcagagagtacaggcccctcccaggagggcggcctaaccaccatgctggcatttctgtggaccatggtctgctgtctcagaccccctccacaatagggtctgcaatctcattcaccccataaatacattctgtctttcctctgatcccctcccattagcagggggaaataaatggaagtcagacggcccagttagaaggcaggcagtggagtaggaaaatagatgatggtggtttggggagcctcacatcactcatggggagacattcattcccatgggccttccaatcacccttttctccaaatctaaggacacaggacaaatgggtcctcatacaggcaaatatcttaaactggtatgtgtattcatttatagttctaatttatatgtgtctttattcacatatattttgcttctggagaaaagctcaattagaaaaattaatacattattcttcttattgcccttcagctaaaacaagcatacacacccctcccctttggattttttgtttagcaaaaggttaggcctggcacagatgaaatactattcagagttcacagtgtattttcatttcataatatatttgattttcaggtcttgaatttcacatcaggaagctgatataggaagctgaattcagccagattttaatacgaaaatacctctgatcaaggcataaaattgtactttaaccagtaaccactgtatttctctaagctgtgaaaaaacatgcattcattaactgctttttcctctgctgtcaacacagtcaatacatgtgcataactccttattgtctacatggtgattatcttgctgatgaattctcaaaggccagagatttggactattttttctctgtaaccttgcatgttcctggccacatgccaccaccacccaaacagaatgtacgcagggaatgtatttttcaggataacctaagaaaaaataggattaagaagataaagctgctgatcatgtaatgtactttagactcagatatataaatatttgtgaattatctgtcctatttctttcttctattaattcattgactctagatgtgcattggaaggctagggagaaatcaggggatcgtgagaaagagcacagaagtctgcatcacacaaacaatattatttcaagagccatgaactagatcctaagcaactcataggcaatgacctcatttcatacctctagtctctaagaaacatataactggcctgaggaaggaaaatgtgggcaaggggtagaccggggtcatgggtggaggtccaaatagtaatcaatggagctcatagggtggactgatattgaagctgctatgagccagccacatgctgggcactgttacatgtcatctcatgcaatactcccaattacctgcctagtaagcataattgtcattttatagaattaaaaacagactcaaagaggttgacagtctaatgtaacacaacagctaaatgggggatctggaattataatccagagctgcctggctctgatgagaaagctctttctgctgtcatatgcagcccacattaatagggggctcagaaagtattctctggataaattatataatgaatccaatgaaggaagacattattttataatatgcagcataataggcactattatgattggattttcctgcttgaaagtagctagattagagtaggaaaccaaaaagatgtgaattcattcagtcattcatgcatttgcatggattgagctacctacatttgaataaatgctgttaatccctgattccttggaagctcacattggagagataagcatgtcattaaataatgccataatagtggtatctcagaggactagcagaacataattcaatctgacagagtagaaacagattgtacaaatccaattcaaaacatcataaatcctctaagcactgtcaattcttcctccaaattatctctgaaattcctccttctttcccatttatggcctccatttacagaagcgtgtactgtctctcttagctgtttgccaggccgccagtctcttgctgttcagctctcaactgcttccagcaagatctttctaaaatcccaggcttgccaagacttagcgcccacagctccacagtgactcctcattgctgttaaggtaaaggccttcccagtctagcccttcatgcttcttccatgttctatgggactgccccaggcttcccacctggtaccactgagcctttccatccttcccccactcgactgccaggtcaacacccacacccacgcttcaggactcaggtcctatgtttcgggccttcttctgtgcaccattcccttccctgtagcccttgatcatgatttgtttatacgcctccgcaccttcatggccctgaacccctcaagggccgaaactgccttacttttctttttgacttcccaacttaccttagtggagctgtagtcacatagaatagacgctcataaatgcttctctgggctgtaaaggttgaattttccagctaagcaaggaagaaagacaatttcaggcaggaggaagggcataagcaaagtgcagagatgtgaagctcaagagaaatggatgggctgggcagaggtgtggctgcagcatcaggggagaagaagtagtgcctggagtcagcaggcacggcttgcaaaagcttcacctataggtgaaaggacaccatctcttgcaccaataggctctgtgattggaggcaactttgctgttttactgccagaaaactgaggatgataacccaaactgcagttcaagtggcattcactggtgtggctgaaatgggtgtttgtggccagaatgtggtctgattggtcagtgcccagctctgttgattagcagatgttttgaatatagtagcatccatgtgcccaagttgttgggatgattcaacaagaaactttaagagctcaagtgccctgcagttgtcagccaggtgattctcttcctttggacccagttagacgcaggcattacctcgtggctttgccccagtgtgaatctttgtcctccaacttgatctttttatttgtttcattattgtatttaagttgtttattttagagacagacattttttaacagctgtgcatttcctgtccctttgttttccagtcgtcatgtgtttccttactctctgtgggtgaacgtttcagatgtctgtttgcggtgcccagcgtgcaagataaaatttattgcagtgccttcggcctctaactcaccattccaaccaattcagatagcccaaggctgttttatccagtggatttttccatgtagtgggaaataaatcttgaatgttactgtttagattagccaggaaactcattctgggatgtttgcccacatccattggcatttctcaaaaggaaccccaggtgtctaccttgacaccagcagggccacttgagccctccgctggcattcatcgcccgctttgttctcagcctgagtttaggagttacagatgtgagaggcgggattatacagccaacatctctaagcgggcagtggctcccttaccctcgaagacctcactcctagcacgtcctggatgtattcgtcaaaatatgtcctcttatgccacgtcagcacagggttgctccccactttgatcatcaagtttaaacaaaaggaaagattttctttctttctctgcctctactggacatcatttcccacctaacagataatttaatgtatctgttactgaatgtgtttgaattacagacagagaggtcacagttaaagaaggaagcctgctgctactgcagcttgtcctcccaaggaggtgtttgatttagctgtgtaaacaaatgactgcattctccagaggtcctgaacacagctgcctgcgctggagagggctcaaacctcttccgccagggtgaactctgcttcctggtgagtgccagcaaaacaaccaacaaagagctgtaggacttgtgtggacttcaaatggtggtggtcctgccacttgggctcagccacagcagttaggaaactaaaggggaggaggaaagccctttccttgctttattgtcattggctgtcatagggcattacaatggttctctttgagattctgagctccggctataacatttgcccagaatctgcctctgaggccttaagacactgtgtttttattcagcaaagatgccctttgactccttttcccactagtggtgctaggtttgagcaccttacactggccccttacaatagccagttcttgtctacctacattcttccctaacattcatgattgcatagttactcttagtgtagaagcagacagcttttacacatagactccatggccgtagcctcatagaacctactatattctaacttgcaagctaatcagaccaaatatatcaaaatcaaaaacctctgctgagagtttattcattcatctctgtctcccaaacgtacttatgtacatacgtgcactaatatacatgtccattagccaagattttgatttcagggatcaaagcaagtaccaatagggaatgaggtcacttgctgcatggcaggtggcttccccatgagaatgcaaggccacctcatgactcatacttcagagggtgacccaggaacttctgattcatgtccaaagcagcttctacaattgctctaccttgatctagggaagatgtggggaggatgacattcgggattagctttataaggccttcctgtgggcagagttgtctgactttcacctagtgatcaacaagcagctagcaagcatcagtgtgtgaggccccacgccctctcagctcccctactgcccacctgggacatgggctttggcatctgtccatagcattgttctaaccaaatgaggtgttatggatcagctcaggatgggatatgttcccagacatattatttaaagaaaatagctcccttcctcccctgataaacagctgccatggctaaaaggtaacctggctggggcttaaaagtctgttgactttcaagatattttgcaaaaacagtcataaaaatggtatttatcagatcctaactatttgtgagacggtttggtataccatagtggttaaaaacacaggctctttccagaggaggtttactttgcttagtcgtgtctcctaagtgaacttggacctcataaggttgttgtgagaatgaaatgggtgaatatgagtaaagtccttggaccagttttggccgtatagtaagccttcagcaagcatctgcttttattcctacagggaggcaattgtaagcccttcacaaacagcgtctaatgtgatccttagaacaaacctatgagatagggcatatctcaattttgtaggtagggaaacagaagccacacaattaggaaatggcaacagatctgttagactcttaaacactatgctacaccaatttgcaaggcaaggaagacaaagcacctttgaaaatgggtcagatgttttagggtaaatgaacgtttgagaatcttttaagttttttttcccccagagattatcaaggtatcattgtagggggatgcatcaggaaacatgactatgaatcagctgcctgataaaccagccaggatggagcccacgtcatcacagcagtcagcaatgccactgaaaaacatcagctgcttattcccgtatagatttccccttaagacatgaaaagggagttcaaagagaatgggccagatatctctgagagtcatattactaaaatatatttatttttactagcttttttgttttaagaggtatactgtcattagcactgtagcaaaaattcacgttttattaatttctcctagtttatcatgtgattctagggtaggatgcagagttatattcaaaatacacaaatcaactcaactcagtaaacatatatcgaggccctatcatgacaaaatgctattctagagaccacggcgaacaagccacggccccagcctcaaagaatgtactatctttggaactgtgctggccaatacagtaaccagcagccacgcagggctatttaaatttaaattaattaaaagtaaaaacacaatgcctcagatgcattagccacattttaagtgttcaatagatatttgtggctcctgcctgccatattggacagggcagatatagaacaattccatcactgcagaaagttctactgaacaatgctgctctggagcagaagatcttcttgttcagggatgttacacccccgcttgtggctagagtgtggcttatcctcagagcaaggataggggaaccatggcactctgcaggctcagcactgaagacacggatgcaggctctgcttctgacctagattgaccttgggcaaggccctttgctcctctgatcccaatttcttcaccagccaagtaagaacatcagaccacaagccctctagggctctgtccaaatgccccatgactgagtgaactggtagaacattctatgtgtgtgtcacaacatgaagagcaaagactttcatctccccaaataattttgtttttcgttttaggaattaaatttcagattcactctaattgccaatactaaaattctctatatgcagttctaaacttgacaaaccaataaaaaaagattatttgactacttatctttgtacaacattgaggtctccctaaagcaaatttaaatgcatattttaaaaatgtattctagcagttcagttcagaagccccctggcccaagcatcacactgtcaatcctttgtcctcaagcagcatggttgggtgggttaagtactgacaaacactgggtgtcaggcccatggtcagggactgtgctaacagtctacatattagatgccacctacccccaccctcaacagacccaaactatttatccaatagcaaaccttgcattatttctgtccagaagaaacaaacatttattgacaacttttggtgtgtgacctgtttaagtcctacatctcatttaaggactggtcaatgttaggctaggcaatgcctgtttgtgagagaatcactgcctaaagaaaattctccatttcccttagctctatggtgggtgactacacatactggtatttcttaaagaaataccaattccatttccttttaacataattattaatatctcattagcatggtgtcactgaagcctgggcccaaagaaataccaattccatatcattttaagatcattattaatatctcatcagcgtggtgtcacttaagcctgggccctttagaatttttcatgtacctgtgttcctctgcccatatcagctggaacactaatagttttcttcctttttatctagaagactgagaacattacatgggacctgcccccagggcatggaggctgaggtgggacagtttagttcaggaggcccaagaagtgttgggtgtgcagccccttgttcaaacacagcctctgaatcgccagaggcttccggtgcatactctgaggcgcaggtgggactcgggagtgagaggtttcggcgaatgaattgggattgcctacttcttcccagtgcagtggagcttggttctgtggtcaggtccttacgccctgtctgcctttctcgtttctttatttctcgggtagtagttgtggaatcaaatgacctggggtttgatacctactctaccacgcctctgggggagtcactcagactcgttgaacctaagttccggggctgccaagtgaggataagtagtaattgctgatccacctacttgacaagatagtagtgagggccctgagcgccaggctgtggatccagcctttcccacggttcctggtgtggcaggaagaactctaggcctgaaggtgaaattggggagggagtcccagctctgccactgtctctctgggtgacctcaggcaggtctcctcaaaaaaataagatactttataaagctcagtttcctcttcagtaaaatgaggattccaggtaactcacagatagtttgtggggatgaatctgttccttaaagcctgcagtacatcaataacccagtcttcctgcttgctttcccccctctccactaccagtgatcatagtctgatcccataggtgatatcccagctcaaaaccctacattagcttctgtggctgtttaaggcctgcccagaactcccctggtcttagcactgaaagcacgtgtccggggaagccctgcattggtcgttcatactactgagtcccgcagggcaaaccgtccggtcccaccctcctttctagtgctgctgtcacactcacctcccttcaccctacactcccttctgtgccttgcaattacctagggagttttttacaagatatggatgccctggccctgccactagagattctgatttaattgcttggggtagggcctggcataggtatcttttaaagctccgcagtggttctaaagcacagccacagatgggaaccactgatctattcttgtaggtccccagatacctcatgtgctgttccctgtgcctgagctgacctttcccccactttcctctcctcggctaattcctgcttatcctcctactcaggaggctcttcctccaggcagccttccctgatccctccaggaagacttagctgcgtccctccgctgggcttccccaatacactgggcttgctttcattagaacctgatccttccacattatggttgttggtttgctccaatcctctccctcattagctctcaactttctttcaggaagagatgtttatctttccttcttgtattcctagagtcgaccaggctctggcacattgcagattctcagtatgcattcagggaacaacttaatcaagacaagaccatctgacttcttgtgagttacatgctaagaaagaaatgtcgacaccaatagccctcacaatgataggaacaggaggttaaagaaaaggaaatagatgcaaatagcaatataagtgctttaacaaatctatacaggaggacaaccatcatattcaaattttcaaacattcttagttctgctcttttgtgggtaatggtttttttttttcctcttccaggagaagaaaagaggcatattatagaaattcctcctcccccagcattacttgtcacagaattgtaattggaagtgatttccctgactaagttattttggctgtctgttattttctctcttcctccttgctcttccctcagctggccatcctgtgtgtttggagagagccagaaaggttcaaggctaggaatgtttctctctctctttaaagctctttaatcgtcaggctttctgatcttcaaagcaggctgtagccagtgtgaccccactccctcgcctccccatgctggagagtaaaagcctggagtatttttgtcattttgaagacttgcatatttggacagccttggacatctggaaagtgtggtcctcactagctctgcagggataagagcacgtcagcacttccaagctctctggcgcccctacatctggacacgttgaaaaattaacaccagactctggagttaagcaaacattaagtttataggcctccttgcatttgaccatttcctgggacagcagcccttatcctgtgactttctgtgtgtagagttgagtctttgcagttggtcctcctcacactctctcaactttgtgactctctgcag

SEQ ID NO: 18 - RNA, 358 nt, Homo sapiens
  source 1..358: mol_type = other RNA
gtgatgtttcaggaagggctcgctgcatttctccaaagtcagtgggaaattacatttggtagagagaaagggattgagactggactcataaatcaataaaattaagttaaataagaaaaaataagatattttataaagctcaacaaagagtccttgaatgaaagcaattacagagtcacattgtggctaatattcaaaactgagatttaaactgaggactaggaaatagaattggatccttttgaagcgtttaggagaaagattttaagagaatgagttccgagtcaccctgtggtcgggaggtgtgagtgagctatccaagcccgttcccatcctttgtccctctgtgtcttctcag

SEQ ID NO: 19 - RNA, 1817 nt, Homo sapiens
  source 1..1817: mol_type = other RNA
gtgagtctgaagttcgcgatcctcctccatgacacgctaatgggggtgctggagtgggctggggtgggctgggggtgccctcaaggcttccatgtctttagagagagccccagggaccagagccaaattggagagcatggagctctgactgaggaacctgcttctcccaagctccaggcaggcacagatgagtcagtgcagtggtgggaaagggaaaagagttgatgttgtagctggaaaagggaaggggaaaattaaagcaaggaaagtgaggctgggggaggggacaaattccccactatgtagtatgtttggtatgtggaagggttctggtcagaatgtttgcccaatgattgccacatcagcattcattttggactctgtatggccagtaggtctggttcctgggagccctggaataatgcagccccttccctaactaacatttccatgatgtatgctcaatgacaaggcagaggaatgtgttggatgagctcaggacctgcctccctggacactcccatcccaggcctgtatatctgttgaccaggaataagccaagcaagcagcctactgtttgactgaatatggatttggggggtggtagagaaagggccggggtggagggttgggaggctcatttgtcattatagatggggtcagacacactaccaaaacagcagcagagatctacaattgagttcacctaaaactcagtgtggacacaggaaaccctcttttaataactgtccaatgggttttccagcctcagctctacagaaaacttgagataacagtggccagtctgcagttagtttgggttcggacaataggcagagctgggaaatggagccaggggcgaaagcccaggtccactttaggatcaggacgggagtggctggtggggaagtgaggtgggtgtggggaggcaatagggagctgggtcatttggtatgggagagtcctctggtggctagtcccagaagtgcatgctttacgaacatatgcttctctccctagggccaccttgagtgaaaccctcccatgctggaattgggccctttcagtgacaacacacaacagttttcaatagataataatcccaagggctttactagcacatgaaacacagggaaaacgtgtaaagttcacaagaaagtcgttccagtgtatcaaatctatcctgtttgccaggtggatataccagggtctcctccacctgtgcatggctggtggtgggtccagtggctgttggataactgatgtattgatggatcattcgccttctgaaagtgccaaactgattagttattttgtgtgtctttttgtgtaactagggtttgaccttccagggcagactgtgctggggcggctgaccccttggggagccaagttattgctcttaccaccaccacttgcccttgtcagtcctccaccctcttgggtttcagtgtcagcatgtagctgtctactcagatcccatccacatcatcaagtctgcagttttttccttgcaaggccttacagggaagatctttgacatagaggatataattttattgacacattttacttgcagagcattcacccgggctaaccagaaagccagcactctgctataaacaaaaaataatgcttcagggctaacatggaatgtgttaaaagattccagcccattaaatgtccaggggaggttttcctgttttcctttccctccatctgggctttgttctcaacacattcattcaacaaacatttattctgcctctaccaggtacagagcactctactattctgcttctctccttttgctttag

SEQ ID NO: 20 - RNA, 3714 nt, Homo sapiens
  source 1..3714: mol_type = other RNA
gtaagccaaatggagaaggcccagaaaatcttgaatactttggttcctttcccctttcctcctgttcatgtgcctggattagtcatgtggccaccaaggagagcgtgacatctagcttcccagcccttccttttagccaacgtgggagacactcaaagagacgaaatctcctgaaggagccactgtatcacagcatcctcccatctcccacttcctgcccaggggtccatggtccacacagacttcccagtcccattccgtgaccatctggagaagctgctattagcagagccctgcacagggtgatagtgtaattaaagtggtcttctctttccaaacacagaaaaaatcagttcagggagtgttttcctgggcttacaattttaactactggctagagttgaaatggggaaagccttttgccttttcagtagcagtaggggaggagatctggattatttacttatcatcatcatggtcacctcctacatggcttcaccaaaaaacattctgctgcctgaaaaagctccaacacctctctctcttttaaaggatggaatttggagtccatccttcctcagtgataaggagtttttatagccacaggcagcatctattggtctgtcctctgcaaacttgcaactcctctgagagctagacttggaaatgaaacattattttgcaatgcgctgctatccttcatttttagctcctccaccgtagatgatagtttgtacttgttaaatgataaggatataaatttaggtcattttttatattttattgggtggaatttggtataatttttagacttcaggctttacaggctcctgagatggactgattgagcttgttctacttcttccccatcatgataggaagtgctgtaccacactaggcagtgtgtgtagtgaccacagactggctgagtgtctcccatcccatgctggcccatatctggtacccacctgatccacaaatgttccatcagatcctgttcaaacaacacatctccagttaagccaaatcttgccctttctccttacggtaaaatgtactaaatctgaaggttttgtctttttaatgttgctccatgatccagtgatctgtggccttggttatgctctgtgctagagtcctaacaagacaaatgctaaggtagaggtcattctgctcaaacaacctgaccccacctggatgtgggcttacatttgcaaagggcaccaaagttctaagagatgaggggaggagctgagccccttgtccttatctaggtttcccttgttctttcccatccctcagtctgcttcttttcccagtaccaacatgtttgtgtcctcagaattaaaggagtaaaaatgtgtaaacatctgactagcaacagccatgagattttgcctggcttgttgataagcagcattgagatctgccctcctaagaatgggccattaggtcttcaaagcttttacgatgtgaggtaaagaatgttcaccaggagtttcatgcacaaaagggtttctctttgtgggaactagaacattgttccagtgatgacggaaacagggctttccataccaaaacagggttttcctttgaatgactctcccacctttcccttgtctcttcctccccacctcaacaacacaggaaagaagctggaagcagggacaatgggaaggtccctttgttactcgagctattagaaacaaaaagaaaagtggccatctgaggaagccacagctggtgaaactgtagggtcacagagtgaattacacctctggcttaagtcagtgaaaagtcctagaagtttgtggtcctagaagtcctaaaagtttatgggactttgttttgagcaaggataagaaattgatttcaggctgggcgtggtggctcacgcctgtaaccctaatactttgggagacagaggcaggtggatcacttcaggtcaggagttccagagcagtctggccaacatggcgaaaccctgcctctcctaaaaatacaaaaattagccaggtgcggtggcacatgcctgtagtcccggctactcaggagactgagcaaggagaatcccttgaacccaggaggtggaggtctcagtgagctgatatcatatcactgcactctagcctgggcaacagagcaagactctgtctaaaaaaataaataaataaaaaagaaattgatttcattcttctgagaactgcaacaactaccttaaagtgattccatccaaaacccacatgttcagccatggacttgcttttatggagctgcgtgtgggtgacacacaaaatcaggagctctgagtcctaatttagacttttatttagatttcctcaaatttgggttccagttaagcgtgggtctcttctgtgccccgctcccctttgccatttgttttatctgttcttcagtctgttctgtcagtacccacaggcaggagagcagaaaggagaaatggcagccacagcagacaaatggcacattcgttccactcagctctcgcatgcccatcacagatacagctcattggtctcttttctatgagaggaagccagagctccagggaactactgccaactgatcagaactcatttaggacatggacctatttgttcctttatgttcctgggaagagcacaggatgaattctatgtactcatttacgtgttcagagagtaaagtgcctcataggatgcctccagcaaaagataaccaagaaggtctaatacctttgacaatctcagtttatcctatagtgtaattggatagcagttcccctagcaaaagttgctagtttggtcctattttctacatagccaaagtgattgattcattggttaatgtgaaagttactgagtactgccagcaggttctaggaaatatatttgtgtgatattcatggatggggaggatcaatccacttccaagtgatttggattaattactggtattttcacctgtgtgggtagcaaacctcagaaaatcaagtatagatgacggcataggacaggccaggccccaggcaaaatgttgaagctcctctggagttccctcccatctccctcttttgttttccatatacctggtttatccagggccctggagatgctccaagaccccctacccaggtcttcctcccttgtcccagctatatttctccatattaccactcttctcaccgaggatttgcttacttaacacataataaatactattaaaagagaaacttaggcacattaaaatgttagagttgattccagcaaacagtgattcacaggaggctccagatcacaagtggttcagggccccactgaggggtagggaagcaagacaaagaaaaacaaagcaaatatttgattggttcaagtggaaagtccctgattacaggttagtgggcagtttgtgattagttaagtttctctaagttgggttttggtttgctgatgtaggaacacagaatgctggggccgtttcaacctaatggtctcccaattaatttttttaacattactgatgactgttaggagtctaatgtgctactcctcccagggaaaatggcattcctaggattaaaggaactcagcacatggagtgtgcgtagaaatttagacactaactgcaggctggtgggagagagccctttagggcagaatgagaaggcgtccggccaagggcaggagttactgacgcatggcctcttggtttcag

SEQ ID NO: 21 - RNA, 1285 nt, Homo sapiens
  source 1..1285: mol_type = other RNA
gtgaggcccttgggctggcccctgtcctacaacacgtttccttggaagggtccgtagcagtcctggaggcccagcctgccctctgagggggtccactttgcctttgacctaaggttaaaaagttcacgtgaggctaaaatgtacaggggcaaaagtgggagcagtcctcaccccgagcgatgcaacagtgactcctcaccacgcctgcttgattcatctgccctggaaagtcattaaaaaaccagttcaactcatgggtccctttatttactcacaagagagagccagcagcccatttcactagttttcctttcctactctttgagaagaatcagaagggagggagcttgccactttactatctgtctaaagagatgtttccattaattaaaggtttttgttttgcttcaaaaaaacttgaattggagtatttccacaagtatctttaacatgctctaccaatgtttgcagaaagaagtgcagaaatgagactgtccacagagtcaggctcgctggccaggagaggactcccgaagctgacttctgatggcctgagaaacttcctagttcacaattcccagacccagacaaagagcactgtcttttctctaattgttttcaaatgggccatttccaccctctaatcagcctctggccctggagggtgcagttccccttgtcctccggagtctccctgtctctgtgctgtagagtcaagaagggacaaccacctgccctcactgggaaaagacagaaagtctgacttgttctcacgactcacacttattaggctccagaggtgtcagggcatctgcctttcatttcttaggttaaataagaaatcaattgctgccatttgtagtacccaattttctaaaatgatcacaatggataagtggcaagaaatccttatgactcatctgtgggcagagttgggctattttggtaatccttgagtaggcagatggaatttgaggccatcttcttgggtacatagatcactaggaagctataggtctagcaactgtggattagggctgggctgagaattgtttcatgttttttgtgactgtatagctagagactctcttgtttgcagagagacactctgaactccccctggccgtcaagggaaagactgccttcaccctcctgagctgaccttacactgagagacaatggggaccctcttttggccctcccctctacctcgagggcatctgggtgctgttgcattggataaaaggcactgctctttttctgtgccctctccgcctcactgcag

SEQ ID NO: 22 - RNA, 3412 nt, Homo sapiens
  source 1..3412: mol_type = other RNA
gtaagcatcctcctctatagggtaaaggtaattgagttcttcagatccccagccctctccattcatctagtttaaatttcatttcttccaagctctttgtcagaaccagcatttgaagtttaaatctagaagttaaaaatccaccagcaaatcctactggctctacttgagaaacaaatccagaatctgatctcttgtcaccacctccaccacaaccttcccaatgccagtctcttccttccactaccacctcccatcagtccattctgcacactgtattcagggagatcctttcagaatcaaggtcatgtggtgtcagccctctctgtcaaatgcttgcactggcttttcctctctttcagagtaaaacccagtgtctcaaccctggcctccaagctgcttcattatccggcctccaactctcttcttcatcttacgattttccctactcctccatgttcctctgctccagccacgtcggcctccttactgactgtttaatacaccgagcgcatttcctcttcagggcctttccacctgctgttctcatgccagaagcacatttctctccccacaacctgcaacccgcccctcatatctgcaggcttgcttccttactttgttaaggtctctgttcaaatgtcccattatcacagggatctttccagactgaagagatctacataactatggctctgtaaacaacattcctccagggttcctgtccccttaccctactttattttggggaacattcttcaccatctgatacaatgatgtatcttatgcatgtatttactgactctctgcccttagtagaatatgagcccagagagcatgcatgtggtctattttgttaactgtgacagtcccagtgcccagaatagtgcctgacctttggtgggcactgaataaatatctaagtaatctgtagcatggaaaatcagcttctgaaaattggctgtttgcacggtcgtgtatttgcttggtagaaaatcaaattttccttcaaattagcattttctggtaactagagctgccccatcttcctctgagtggtctccaagtcagccaatagccttgtgctgtggcagccatgcctggctcttgatgctgtagccaaaagcaggcaggggatggtgaggctggtccagtccatggggagggacaaactcacagctctcagatcatctcagggcagcctttgttggcagaaataggtaggcagccaccctgaataggaggaaggcttctagactgggtcaggaggcctgggtttgcatcctagtggcaagcgtgcattcatttactagggctgccataacaaaataccactaactgggcagcttagacaacagccatttatatctcacagctctgaaggctggaagtccaaaatcaaggtgttggcagggccatgctccctctgaaacctgtaggtgcttgggcactccttgacttgtagatgcttcctgctgatccttcgtctgcacatggcattctgcctgtcttacatggccatcttataaggataccaactggattggattaggtgcctaccttgctcccatgtgacctcatctcaactaatcacatctgcaatgaccctgttcctaaacaaggccacattatgaggtacctggggttagcactctggtatcttttttcttgacagcacttctgacaccaaatgtgtgttttggttttttgttgttgttgttttggcaccaaccaattctcctatattaatgggttgtccaagaattcaattgaattctgacactatccagaattcacacagactccacgggttcagtcccacaaggcttccccgtcttcagatgccagctggaaatgtggtgcccaggctacccacacttttgccaaaatcctgtacttacaatcacagctttaaaatgaaggatgcagctcaggaactgccacatggaagagaagcacagtatggggtcgggggaagagtttctatgctctctctagacgcaccactctcccagcacctcaaagtgttcagcaacccaaaagctctccaaatcttgttgttcgagagtttttataaccctatctccagctccatactcccccattggaggttgagggttgggactgaaagttccattcttcacatgtgtggtgtttctggtgaccagtccccagaaactgcagctatcttggggctctaccctgagtcacatcattagcataaactcagatgtggtagaggaaggggcttattatgaataaaaaaagacactcctttctgccaggaaattccaagggttttaggagatctgtgccctgcacaggagctggggacaaagaccaagtatattttgtgttatgccacagaccccaacatgtctttttggagggagaccaaattcaacccatgacagtgactttgaacaagacatttgaacttagtctgttttttctatcctactagattgttggaaacagatataatagatgaaaattagttgattaaaattgaaatttgtgcataattcaaaagttttattttagccaagctaaagctttcatttattcaacagctatttactgagcagcacctgtgcatgaggctcagcagggccaggttctgggaacagagcggtggagataaagatccagacctgccccgaggaatagacagtccagtggcagcaaaggccatgaaacatacggcaactcttaaaaaaagccgagaccatgattttacaaaatcaacattttgtagggagcagaactttcaaagagaactggactagaaatttgggagtctttttcttggaaccctggtagatccagtagaatgagggatgggggtgtagggttaaaaacactgacattagaactggattacctgtgttggaattcctacatttctgtttcactatctgtgacggggggcagatggctgaatctcagtgtgcctctgtttcctttctcacaagaataatattactacctatctcctggggttgttttgaggtttagattatttaacacatggaaagcactcacagcaatgcctgccacagaaagaatatccagtacatcttagtgatgatcaccattattattatctgactcctggaaaaggacttgatttaattctctcatgaaacgttttcttggaaaactgatgtcaaccaagattattggtcttgctgttgcttataacaccccaaaaacatgactgtgtggataaaaatatgttggaaggggtagtctttctgggagcctgagaatagccatgtaataataactgcaaatatctatagttacaatttgaggttcaggtaaataaactctagatcttatagaactgcggtaaggtaggatagggagactccttcgactttctctgtttatttgtctctatttttag

SEQ ID NO: 23 - RNA, 2675 nt, Homo sapiens
  source 1..2675: mol_type = other RNA
gtgagtcctttaaaacacaaatcttaatgtttgaaatcaactccttgggctctgtgcaagatgtatatggatcacagaggtggccctctatgtaaacggtgtgattcctgatgagtcagctgcctcctggggctctgccccttgatgggcattgcagcgtctgggggaccacctttcacaagttgctgggccctgtgtgatcatgaatggctgatcatggatgaagccctgggtcctgtacaccttgtccagtagactaaattgccctatttaaaaaaggccaagccacttcagggttcaaagaacttttgcagcttttcagtataaagcagaaatccagggaatcatgaaggaacctttgcattcatctcccattgccttccttgtgcctttttattcttctctgccttttcaaaatataaattagtttattctcccaagatgaagactcctcctggggctgaggcagagctgttatcttcagggcaatacctcagattctcctggtgttgatctttcttaggggtggggaaaaaggctgaaagggcatttgcccacaacacatcttaggtaaaaggcacctttactactgaaccaaacaggaggcctagctagagaaagttctagaagcagggaaaagcacagactcttttgtgaggtctgagaaagcaaagaaattccagggtgaaagcgggggactcccctagagctgaagtactctcccatctgtttgttgctcacctacctattctttactttgtattattgggcctgggccaggacttatcctgcaagcactgagatggatgtttgttttctctgggggattagtctttttttttctttttttcttttgttttttgcttttgttttcactgggtcaaacaaacaacactttaacagctcaggattttttcattgtattgacttgtctacctgtaaacttgttaattttttactataataaaattatcatataataaatgaaaaatttcaacacagggcttgtgggcattttatttttctctacaatcccaacagatactctgcctcttaagaaaaaaagaaatcataaggaaaatatgctccttcaaaagtgaatcacaaatatgtttgccaacggaaggcaaatatttttcacctgtctcataggctggactgaaatggatttctaaaactctctaaaaccagaaaagagctgagtgtctccacccaacctccctcctttcacagattaaaaaataaaaaatggagcccaggagacatccagtatcttcccctattggtcacctgggacaaaatctggaacatgcacatgcattgcctggcaggaactcattccagtgattaaactcttcaggaggatgtttcctcttgctatttcattacctatttgtgcagtttgatagctagtaaagtgatcaaaggaactgtggggcatagattcaaaagtccttcaggaagcagaaatagaagaacagtactagaggcagcaggtccctgaccagcaggcccactacctgctgctccagcacacatcctgcacattttcagagggtgggggacagaggggccctgggtggctgttgcattgagaaatctcgccctgctcctgtatgtgcacttgaggccgagagcccttggatgcctggtgacagtggtttcctcctgcccctgccttcctctctggcagactgactggcccttctgctcctcttccccttccaggatgtcctgatatctttttaaaccaaatgccaagtttgccaaaaagtgtctgtttgtgtgtgtgtgtgtgtgtgtgttcaatgcgtgtgtttataccacacttcacaatttgtccaggcttgtattaataccatcaccaggctcaaccctggtgttaattccaagatacttaaatgcccatctaggtgaatttctcaggtaaaccatatattcaagctgtagtttaagctggctgcccgtcatagcactttgaatagactttgtttttgtttttgttttttgagacagagtctcactctgtcggccaggctggagtgcagtggcactatctcggctcactgcaacctccgcctcccgggttcaagcgattctcctgcctcagcctcctaagtagctgggattacaggtgagcgccaccccacccggctaatttttgtatttttagtagatacggggtttcaccatgttggtcagactggtctcgaactcctgacctcatgatacgcctacattggcctcccaaagtgctgggattacaggcgtgagccaccacatccggcccctgaatagacttttactcaaggttcaccatgactttcacatgttttgtattggagtaaaatgtgccagtggtgggctaaagaaaattaactcatttcaaattcaaacctggttttcttaatttttttaaaatcacagtttctgaaactgtgggctcctcatggcacattgagaggaggaggtgaaactctccaagtctgaagctcctgttataaatcttcctctggcaaagattgtgtgatcaggcttgagtacctcacagtcctagagcaggtcaaaggctggctaggaaactcatttgctccctgtacctctcccctcctttcctgcctttgctcgttctcagctcccggtggtagagtaacactggcttctgattggtgcag

SEQ ID NO: 24 - RNA, 1774 nt, Homo sapiens
  source 1..1774: mol_type = other RNA
gtaaaaccccgataaagaatacacagcagaggcgaggaaaaggctctaagcactgcagagggccagagcaaaacatctcatggcaagggtggaaagaagcctaggaaactgactctctctgtggacaagtgttaaaccagatcccttctcagaggtccatctgcatgtgtgtggaatgaatggttcagcccagacattagcgcatatttcctggagaaagcaaataccaactatgtagtgtgcctgtgcccttgttaggcaaatcccaagtgagttgcacaaatgtgctgacttccgaggatttagcaagaacaataactttggtcactgggacttaaagcggatatgagctataaggaaagacaaaaataaatgcttctgtgtccagggggaaagagactccaggggagctgactacacttcacttacggcttacaaatctagaaggccattcattgaaaccatcagaagcctttcctgacagtggaagttacctaataatccctaaactgacgacccagatttacaagttttgttttcctggcttttgctgccctcatcttctctcttaaactagttctgtatttctcccaaggcttttcattccctaagcatacgcatttctctgtggccaaaatgctctgggtttagacaggcagcacagcccctgggctctgcctgacagggcaggagagggtctggcctttatccctccagcccaccccaggggccatttcataaaactaaagccagagacctgcagcccctcccagagttagactgcagtacaccatgcctctggcaagatcctcctcccacagtggaaagtctaagccaaatcaggaggctggggactggttccacctcagttgcaggcaaggccaggaggcacggatagaagaaacagtggactttttccccctagggaaagaaatgcttagagctacagtattaagatgacaaattaagctgtgccatatagggtgaaatgaagcagggatagatgggaggtcagggagaagtgagagcactcggtgagggtctgcactggagggggcatgggaggaagaaggaggggagtggggtttgagggatggtgatgaggaagcgtggactgccctacccacctattggaaaacctgggagttctgaggagcaagaagccttagtcaaagtcaactcaaagattcaagccaaggtgactaagagaatggcggtccagaaaaggtcatgggagaatctgaaggcagatgttgttttgggaagatgaagaacctaagccgcttccagaaattcatgaggaaatgccccgtggactgttggcaatgagggcctaggaccaaggttgagcttggggccaactctccctatagacagtgagtgcattctgacaagcatgggctctgggttcaaatcccaactctgccactcatgcctatgtgtccttaataggacgcttgatgtctctgtgtctaaggtttcctggactatggaaatgagcctaataaatgtctaccccttaggaccattgtaagagtacattgaggtaatttgtgtaaagcagtcgaagcagtgcctggcatataggaggtgctgtataaacgtttgatgctagtattactattattattctggagtcttccttgcaacggtgatagccgaagccacaggggcaggtgacgttataggcagaatacaagggcctggagacagagccctggggccatgtaattaggcattatgtttacatcatgttcattttttttcctccaag

SEQ ID NO: 25 - RNA, 2174 nt, Homo sapiens
  source 1..2174: mol_type = other RNA
gtgagtcttccagcagagaagctggctgccatgctagcctgtcatttcctggcttagtctttccctatcagcggctgtctactctttcccacaaattttagtgacaaatatttgcggccccaaaaatgtgtaaaagctttctgcagtattcaaagatcactaatatgtattctcttgatggggaggtagaatacgtttattgccccttttgtgtgccggggaagtggacattcattcagagagttgaagtgactttcctgaagccaccaagttgtcatggctcagcgggggcaaaagccaggcaccacagttgcctcttgtttctcacaccttgagtctttccccccatctcaacagtccatggtggtgatcaagtcatggccactgtcatcatgtgcatggaagctatagagtcctcctatttcctttctcttttcttttctttttttttttttttttttttgagatagtaaccattacccatgctggagggcagtggtgcgatcttggctcactgcaacctccgcctcccaggatcaagcgattctcccacctcagcctcccaagtaggtgggactacaggtgcataccaccatgcccagctaatttttgtattttttttttttttttttttttttagtacagacagggtttcaccatgttggccaggctggtctcgaactcctgacctcaggtgatctgcccgcctcagcttcccaaagtgctgggattacaggcgtgagcgaccgcaccaggccgagtcctgctattttcaaggaacattccttttcctaccaatcattaggcaggcttcaacatcagctgatgagggttagtggtcgttctggagaaagtgaaaaaagaatcagtctctagaggggcttgtggagtaaccgcctggtaacagaaggtcagggcagggaaggcaaaggggctctgcgcggatctctcagctccgcaggcgccccactctcctccaagggacccgagcgccatctgctgagaggagaacacggcccgccatggtttcccaaggagcagcagacacggacctcgcagggggcagcgaacccacgtgacacagtcttcaagtcctttggagagccccaggaaggaacaacagcgtgtacaccctgtgatggaatgttctctagggcggttcagtgtgaatggaatgtggggccggtgccattctaattggttctgtttccctctagtggttgatcgcggagatttcggcttctccatcaggacaagttcagatagcctgagatggtatcagaactcagggacagagctgggtgtggcggccctgcatccatctgctttctctccatgctaactgatatggtcagagagctggaagcaaattccaggaccccagggctccgcaaaggcaaacacattacttcatcggctgctgacatgcaacttcccccaggggttaaaacaatgtttaatactaacagtaataatatttttgagttttactttatgctggcgctgttctaatgttgtaagtgtattaactcatttaagccttacaacaacctaaggacatgggagtcatagttcccatttaaaaaaaaaaaaaaaaaaagcccaccattgctctgaggctttttatgttttggatccaaagctaatattggtggtggtaattcccatgcctggcttcgatcaattaatcagcaaatgcctaggactgcttagggttctggccttcatcaagaccttacccgggctttatgatgatgacacctggcttttcaatagccatgactgctcacccaggaggcaacgcctcgagtcatgcaccgaacaccttttattgatcctctccaacaccaggctccgtgatggctgagctggggacacctgtgactgcacgtgaacattttgaggctgggaatcccaaaggccctcggcgttggcctgggagcaccatgaaacaagtagaagcagagaaggatggcagaggtggccctctgcattagggcctggatgtatacactggtgctaagggggccccacagctaataggggtttgagtttgactgacagccccaggcaggaatctgtgagagttctcactgaacctggtgtgggggtggccctcctaaggcatgttgctaaaggccatctcttctgccactgacgcctgtgttctgcag

SEQ ID NO: 26 - RNA, 1137 nt, Homo sapiens
  source 1..1137: mol_type = other RNA
gtaagcgacacaggaactgagaccgccccatcccctctcctcacctctgcccccagcacacttctctagagcccagctcaggggtgccaggcctgggcacaggcagagatacagactcttatttggtttcccctatgtttaaagtcctttgtcctacttgcagtgagaattgtccctgagaatatgggactctgcctctgctgctcagagctgagggctcctccctcagaagggtgaggctgccttcgctctgacagagcagctgatcgatccccgagccccttgtgcagccctgaagtacttcctctctgggaccaaagacaggagaaccattgttcctttttcctgttgaagccacggcctgaaaggcaaacttttcagggggcttttcagttactttttttccccaataagatatcttttatttcttatctaagaagctacgcatagtcattgtgaaagaaaaaaaaggaagggaggaaggaagggaggaaggaaggaaggaaggaaggaaggaaagaagggagggagggaggggagaaggaagcgagggagggagggaggggagaaggaagggaacaggagggaggaaaagggaaggggaaggaggaaggaaagggaaggagggaggaagtaaatataggtaaacaaaaaattgaaaataaaagtcacctgtaatttcactactcagagataaccgctgagttataacattggtatataattttttagaactttctcctatacatgtatagatagataaacacatatacttcaaaatgataaagaatagtaaaactatgcatacaattttataacctgacttttttttcaaaaaaaaggattgcttttttaaacataagatatcaggaacatctttcatgtcattacatattcttctataaaataatatttaatgtttacagattattccattgtatgcatgaactatgtaagccatcctcttattagatatttaagcagggtctgctatttttgtattgtatcataaacaccaccacagtgagcatcttgattgccaaatcaagaatacttgtcctcaattatttctgtaagatcagctgctggaagtggaagtgctaagccactgcttttctcgttgtcccatcctcctag

SEQ ID NO: 27 - RNA, 437 nt, Homo sapiens
  source 1..437: mol_type = other RNA
gtgctcagagctggatggagacagggccacagatggcaaatccatggctccccagtgcacccaggaggcaggggaggcttggagcaggagagcttctaagggtgggaacacctctgtgaagttacaccaaaaatctaagagcagcccccagatcattttccctgcagagcactgtctcacagcagcctgggttttatttgtcctgagattgatgtgcttgaacagtcttcaaagggtctgatccgaggaggtgagggttgccctttctgcatttacaaagcctgaacagtattagggctttgaacgctataaacatctaagaggcagcaccaaaccactgctgggttaaggtacccccacaatgccacttgccctgggcctttctcttcctcaccctccacagccccttaactctcccgtccttcttgtgcctccag

SEQ ID NO: 28 - RNA, 1358 nt, Homo sapiens
  source 1..1358: mol_type = other RNA
gtaacagctgctgctcagtctcctgggctgggctctcactgcagccctagctgtggtccccactctctcacctgccattttgtagctgagtacaggaaccacaatgactacactcagaagggggtttatcagtgacttggtgaatctaagttccagctaaagcctcctgaggtttttacaaatataaacagagaatcactgatgatgcaacctacttcccaaaatattttagaaaattctcttgacctgcagcccttctgtctggaataatggatgctactctaggtgaatgtcttctctgaccatggggacccaggtcacctgcaaacatacctagaagctccatagctgtcagatgaccactcaggaccagtgtgagggtgacctgctgggcattcagtgctccagagggtggccacagatggaagtggctcctctgtcatggcacctctcagacaaggggctcagatcagaagagacagcaagcagagctgagtgcccatagaggtaacagcacggttcaaccccgtggtcaagccagagctttcccccttgctctactcacacagcgttgccccgtgcctttctctgagggtttgtcatcctgaaatcctcattgctattttctttctttcttttcttttttttttttttttttttttgagacagaatctcgctctgtcgcgcaggctggagtgcagtggcgcaatctccactcactgcaagctccgcctcctgggttcgagccattctcctgcctcagcctcctgagtagctgggactacaggtgcccgccaccacgcctagctaattgtttttgtatttttagtagagacggggtttcaccgtgttagccaggatggtctcgatctcccgacctcaggtgatcctcccgccttgtcctcccaaagtgctgggattacaggcatgagccaccgtgcccggcctgctgttttctgttaatgacatctccagttagtgagagtatgcacgtgtgtgttctttatgaagagtataaatccagagcttaatgatccagaaaatgtacatatgaaactccctagatgctgaccataatacatgagcccctaatatagagatttatttgaatcagatcctatgctggatacagagacactgtgtgtggcaatgctttacagtatgtaggaagctatgaaatgttagttattattgtcctaatatgctggaatttgctgctgaattagttcccttgggtttttttttttagttaactcctgatttttgcaactatatagccaggaaattgctgtacaccctttaccaacaatgcccaacccagggcaggcctggtgattgccctggcccctaccttgcag

SEQ ID NO: 29 - RNA, 1081 nt, Homo sapiens
  source 1..1081: mol_type = other RNA
gtaggtgtctgcccagggaaggaccctggcctgggtgagaaggagcacacagcacggggctgccactccagacatggctactcacacaggctctcgccaccagaatcagtgtctttgttctgggaccatttgcagaagatttcgatgaacacattctgaagcctcctcctacagagatgctttagccaaaatgaaacaactagctttaaatggtctgcaagtattacatgccagattacacaccagtttggtgcggtttggtgcaacatagaagtgagtgtcttattctgtaaggttaggctgttttaagagcaattggttgagcttcatttcaacattaatattccctaattaaacctgaatttcagtggtaagtgaaaactaagaagaggcctccttgggtgctataacataaaaatgatgaaggcaaaaagtaccaaccagcagagaccacttcagcacatcaggagacccagttttatgtctgtgctgcgaagtgaacaaactgtgtcatcctaggcaaattatttaattcctccttttttttagtatttttttcttcttcacatggaacatgaagctaatgacctctgcttctatttcttagggatgtgaagataagtgagataaagtattataaatgtgctctgggcttcttaagaacaggcattgctcacattcaaatggtcatgattatgatatggcagcattatttatgcctctggtttaagtgtctggctgccgctggggtttcctatgtccatccacggggagggaggcacagaatgtctcccacaggcagaacctacagctgccacataattgatgacaagccaaagggacccttggaggttctgctcctctctgtgtgtgactcacacactctctaggataaaatcaagcgactacaccctcaaaatgctcagatgaattaacagattaaacagtgaagaaaaaaatgtgttgactacacttggcagtgagaaataaataaagcgggcggtgacagcagctggcatcagggagaggctgtcatggaagggatgtgcatcttgtcagtcatcccatccatctgttgcag

SEQ ID NO: 30 - RNA, 2692 nt, Homo sapiens
  source 1..2692: mol_type = other RNA
gtaaggactggacgggccatacttgggttccgtctggcagccatctcccagtattgctgggtgtgtcctgttgtgatgcattttaatgggagcaaagagaacactgggtacttctgcaggtcacacagttgttcttttgctttgagcttctttctcctcttccttcttccttcattcccaaagggattttaaaagtcatgcacctaaaggccctctccctttaatgaggaatacactctgtgctcttacccttagtaagccatcattcctggggtccccctgccctggctccaggccacattccttagtgtctggggagagcttcttctacatgtgtgccgtggcgccctctagtggaagcatggtgatgcacggctcttccagtgaattcgtggagtcagagattgcacatgtggatggcaagtctggaaatagcatacacccctgttatactcctgattctcccctcagcttcccaatttcccagtgattctccctttaattaggatgcactgaagctctcaggggtgcccccatctccaaggagctgcagtggagaggctatcccctctctatgtgagagaatgtgtgagaagcgtattcccacacaggagcaaaactaaacttacgtactgatgcaggttaatgaatggggaaagtatctgcttatcaaagaaaaggcatatttttctatttagcacaaactttttcaaatgttaagaatttactaactgaaatctggtgaagcaagagaaccgggcaatatttgcgttgtctgatcattacaactggagggaacatgctcagagaggcatcatcactgttcatgcacctgccctctctttacactgagagaccctgtgatgaacagaaaacatctttttaggatgacatctctgggtctttctcctagcctgccttgctgtgggtacctatctccctgctctctgaaccttggtcaagaagtttatatttgttttaaattgatactaatatgttaagttactgtgatttgccaaaatcagattggaaacagggcctgcatggctgaatgattcttttttttaaattactttatttctaaataaaggttttctttgtatagaatcgggatgctgtgaatggtgggaaatgcactaaatagttatgccccaaataagaaagggaaaatcatttgaatccccagttagctccttgaaagtcttttcacttaaacacacccacataccacacacacactcacagacctccctcccagatgcccaaagccctgctgacctacagagctacttctggaaaggctgacacatgcctaagacacaattcctgggaatccagcagctttgggttcaatttccttcctaaaagaacaatgaatatgacccctggagagctattagggcagagctgcttccttaacgtaaaggactctccagcctccgtatgaagtcatctcagagctaaagacaatcaagtccaacttgcagatttgacataaagcaagacttccaatccggctaggcagaaggattttggttgaaaaccatgaaatcccttcatatggatcattttttaaacaacaaaaaaagaaaagaacctactgggtgtccacaactctgagagctgctttctgaagagtcatgttttgagtcctggaatccctctccctttgacctgcctctcaagacaatgtgcgagagaactctctcttcaagtgcatgcaagtgaggttttcacagttagatttttaattttaaagtaatacacatttgtacataaaattcaattctgactgtatacatgtgtcagataaacagttgatacctgacacttgttcacagtctatgatacgcaccgcatatcctaccctctcccccagcctctctccatggcttctcaaccccccctctgcatttcctgtgacctgaggattcagttttgtttgtggaggcaggtgcaatcccaagagaaactgtgcaatcttctgagaagttagagtaggcatgtgtgtgtgatttagggaaggtacttctcactcagcttggtcaccggttccaggtttgtgtcttgggcaagtcccccatagctggtgacagaccagaaaaatgaaaacaactttgacttagccctcaagttttcagtgaatgagaatgaaaaacaaccatgagtaagagatttcttaccgagatgatgtaaaggataataatagcagccagcactcacctatgtgccaggtatttctctaactgctttgtgtagtttgactcatccagtcctcaaaaacaacaatgaagtggataccagtattttccccttttcacagatgaggaaagtctaatgtgacccacccaacataacatagtttgaggggacagagcatttcgttgaacagaggaggaactggcacaggaaagttgcatgacccccccaccaacctccgcccccaggttgcacagctagctagtcgggaggactttgcttccgtttccctctgcctctcaatgatgatctcagggccaactaagctaaaagcagacttgatggagcatcagtcctctgaaagagtcactgccgagatacaaaatacctcttcttcaaaggggaagtggagagaagtaggaaatctgggtaacctcacagtcttccagtttctggaaaacagagctggcatcagtcttttttcttgtcctag

SEQ ID NO: 31 - RNA, 356 nt, Homo sapiens
  source 1..356: mol_type = other RNA
gtaaagtagagattccagctggtttctgtcaagtgccagaagtggcggttctttgaaaaagtctaacattagagcaaagttttgtaaaagcaaaaagccatcgttccccacccaagcatagcaactatctttatttttggcatagttcccccatctctgcatgcatacaaattttatgtacttgtggttactgtgtgcttacgtttttgtatttatagaagatgatgttctcagatagagtcgtaatggattttcttcccattatgaagcaatacccaacaaaacagagcttgggttagatttttctgagaataagaatgactaaacaaaattctctctttttttcttcttgacag

SEQ ID NO: 32 - RNA, 4696 nt, Homo sapiens
  source 1..4696: mol_type = other RNA
gtatggtgctggagccagtggcttgttcccttccttgcctccctcccaagttccatctcgaaagtctaaggggctgggcacagtggctcatgcctgtaatcccagcaatttgggaggccaaggcagatggaccacctgagttcgagaccagcctggccaacatggtgaaaccccatctgtactaaaaatacaaaaattagctaggtgtggtggcgcgcacctgtaattccagctactcgggaggctgaggcaggagaatcacttgaacctgggaggcagaggttgcagtgagcagagattgtgccactgcactgcagcctgagcgacaagagcaaaatccatctcaaaaaaaaaaaaaagtctaaggaaaaagtcatgaaacaacaaagcaggcaaatactcctccatagtatctgactccccagtagtaggcattttgcatcctagatggctttgagtgacaaaggaataacagactgagttaggtctagatggggacactttggatgaatgaggattcttacggaggtcaggttggtagcttcatccctcagctcctcatgctgtatccccagtctctcggcctgccatgtcatcatcctcatctcctcctgtcatctccaccaggcctctgatccatctctgtctgcatgagtgacagctggcagagtccttaatgtttatcaaatacaactcagacgtcagtctcctggcccctttgagatcaacataaaatcattttgaacccttatttagtggtctatgggctttgaaaacatggggaccaaaattcctgtggattctagaagtctctcttctacatgtgtcagcctgggcaccaactagctccttccatgaacttttatcaaacccacagccacacaaagcatgtgtgagtgtagcagagtttacagcagagggtggagggtggggagatagatgtgtggaagggttacctgccacacaaacagaaaccacttctgatagaacacgaggtgtccacccacactgtaaaatcctctcctggtacaggcaaagctttgcagcgattctcctttgctgcccctgggctcctaacacctcctaaaccaccagttacctccttctttccagtgtggcatatttcagtgttttcctgttggagtgtttcctttctatgtggattctggaatcagctcttaagataacttggttttcatctttcttcataatgatcccaaacatctatctactatgcctagaactaccaatggacacatataccagcccagatatgcttcagcccatcccagtacatcgcatggtgaccaaaagatgtagtcgtcctggcacagtgggtgtggggcaggaagcagtcctctccaggggacagcagcaattcaccacagaacccaagtttctttcaagctctgctgacacagaaattgaataatctcagctcacccaatgtcaaagactcatattaaccaagaccagaatgaaaatatgctaatttatatcagaagctttgctggattcaagagttagggccttttacctgtgcagaatattccttcttgataaataggccctctcaggagaataaattacacatcagaggactgtttagtcagcataggcatagaacaggatgttccaaagatacagtcaaggggagtgggtaagagtgtagcctctggagtgaggccgaccaaatatcaaacctgagcttcataatttgcaaactaactggctttgggtaagtacatagcctctttgtacctgtttccccatctgcaaaatggagataataatagcatctacctgtagcattgttgagagaattaagtgagttaatgcttgccgacttataacacagtatacgatcactgattaagacttagcaactctaaactaaatgtttacaaaccatctcttacctcaaagcacttaacatccattgtcttatttgattatcactgtaatcttatgaagcaggcagggcaggggtctgccccatctggggggaactgagctcacagaggttggagggtttgcctaaagtcacccaggccactgggtctcactctctggtcttagctctgtaatctaggatgctcaatgccacactctcagccacttttcagatggctaagtacatttgttttgagttagctcagtctcagaggatgacattttctgatcttgtctccagtgtttaaatgaacctgtagctgtgcattggggtcacacaatgcgtggcatggagagggtctgtggctgactgccacggttactacgtgaaaccatcattacagcagttactactgttactgcctgagaacatcattacaagactgaacgaagggatcaacatggaaatgataacaaaaaaaccaaagtaactgttttaaggaaaggctagcatcgggaagaagaagagagaagaagagaagaagaaaagggctccctgcttctaatgagtaaaggcagctccctaagcttctgcagcccttcattatttattgggtaacaggaggaaggagcaggaggtaatgattgggtcagctgcttaaatgatcacgggttcatgttgttactgacagatttcaattatgcctaatcataagaaacatttgtgcagcctccaacaagggtcaatgccacttctgaaggggtgactcatagtcagtaactagaaagcagcagatagctagggacaaactggcgattctgaataggcctggaacccttagctctggccaggtcagtgggctccagtcaggatggagccttcagggagagatcaaagctcagaggtttgagatgatatcagccagcaaagaggaggggcagtagggatcctcccagagggagggccagccatagaagacatcaaatctgagcccggatcaggagaaggagcctgcagaactggggctctggcaccgagaacctgcagaacttcgcccctctgagtgcaggtgccagggctggggctgccacccagccttcgcatcccaggcctggcacgtcataggtaaatgtagttgaaaggatgactgagctgatccaattccctttacaactgtccttgtcctgggggacttgaggagggttaagaaagcagctggggaccaaccaacagtcctctaggctctccatgtccagcaatagttgttcagcaaatgagcattaatcagtgactataaactgtagcttcaacataaccgacaacttgcaatggtttctagagcatgctcccatgtgttatctcatttaaatttccaaaccaatcctgtgaaatgttctttttttttttcttttttttttttttgagatagagttttgctctgtcacccaggctggaatacagcggctcgatcatagctcactgcagccttgacctcctgggcccaaggggtcctcccacctcagcctcccaagtagctgggactacaggcacacgccaccgtgcctggctaatttcttttctagttgtttgtagagacagggtctccctatgttgtacaggctgatctgaaactcctggggtcaatcaatcctcctggcttggcctcccaaagtgctgggattacaggcatgagccaccatgccttcattttacagataagaagtctgagaaaactcagatttaggcagattgagtcacttccccaaatttatgtatcttgtaagaatccatattcaaacctcagtcccctaactcttagttcattactttttctaccacttctcagtatcctctaagaattcagaaagaaccacatcgactctgatttttcatttgtttaagtacacaggtaataggtgaatgtattttgttgtttaaaaattcatataatacacaaaaggctaaagtctcgcttcccacttcctctcccctttctacccaactctgcctccccagggagagcttctgctgacagtcggtggacattctttcagagttttacaattatgtgtgtgtgtgtacataagatgtcagtttttctttgtgtaggatacatgaacatgaattttaaacataaatgtgagtgtattacacatattgaccagcaccttagtttttttgtttgtttgtttggttttctttgtgctgtttgagaaggagtcttgctctgtcacccaggctggagtgcagtcttgcaatctcggcttacgcaacctccacctcctgggttcaagtgattctcctgcctcagcctcccgagtagttgggattacaggtgcctgccaccatgcctggctaatttttgtatttttgtagagagggggtttcactatgtaggtcaagctggtctcaaactgctgacctcaaatgatccatccacctcagcctcccaaagtgctgagatgacaggcgtgagcctccgtgcccagccagttttgtttttttattaaccaagttacgtattttaaacttctccatgtcaatgcttttagagctattttgttctctttaatgttaatagagaattttaaggcaatttcaggtgaatctatacaatttctctgtataagtaatttacactagaaatagatttttataaagatgattaagctaccagcctggtatttcattgctgacttaaatgaagaggaaaatcaatgctgtaagggaaaaaaaaaatggcattagagatccagaccttataggcattttccaaattattaattcaatctctcaaaacag

SEQ ID NO: 33 - RNA, 657 nt, Homo sapiens
  source 1..657: mol_type = other RNA
gtactattgtcggtcggtgtttagctgagctcagtggctcctctcccagccttcccctcctctcctgagtgttccttcaggcatgggttataactcagcaaggagcaccctctttagattctgctggttttgtttcctgctttccaaacccttatcttgattcttggtaacatgaatcttctttgtaagttggacctcccctagcaaagaaaatagaataatagtgaaaatgttaatattgtttttatttttacagtgagggataaagtcatgttttcattcatttttgcagtgaccctacatatcaaaatcattgccctcttttttcttttaatgttgtttaatttagaaaaagaagctctggtttaaagaacagtgagtcacgtgacttgctctttgaaatgccctttgaagtctggctgaacactgggctgcattcagattcttcagtggccaccagaacattctgttttcttctgcacatcttacctttgcacaccctgcttattatgttcccccagaagcccaaccctctccaccaggggctgattaggaggctgcaggataaatgtttaaaagaatgaagatgtgtgtgcacgcgcacgtgtgacatctccatgccacagtcatgtttattccacgtctattctcccacag

SEQ ID NO: 34 - RNA, 469 nt, Homo sapiens
  source 1..469: mol_type = other RNA
gtgcgcggactcggggtcaccattctcctctgtgggtttggggcacctgggtcacatgctgcttagaagggccctgaccttcccacttcactgggaccttcaccaatgagagaggggaggggtctttgggctgcctgcagaaaggaacttaatgtatctgccactgcttggaaaggcgatcctagtggacaggcaggactgcttgggaaggccgaatggggaaaggaatgcaaagcttaggtgaatgggttgaagcgccatctttttgaggcataggtgacatgccatcagaccactgcgagtgttcaggcagcctaccgcactcccaggagagctagcgccatcccaaggcagcattcggtgcctccaatacatacctggcacacagcagctatccagtaaaggctctgagttgcatgatgttggcacgcgcctgctctgtcccagtcacatgtctcactctgtctag

SEQ ID NO: 35 - RNA, 796 nt, Homo sapiens
  source 1..796: mol_type = other RNA
gtaagtgcctacgcgcccctgtcctaagaagactagctcccctgggaggacccaacggtgggttcaagatggcaggcgttggggaggccccactcaatcctgctctgctggtcacttccatgtctctgaccagcactcccccaacctctccttccacacttgtgtgcagggacattcactacctcctaggaagcccccacaccactggacagctctatatttctcagcatagaagttctatgttgagttgacagatgattccccataacttatttgaaaggcctctgagcagggagggagggaaatagggttatgctattgtgtgattgggccttgaatggcgtgagtgacacagtggccagtactttgtgatagttgtgagtctggagaagggagttagcgaaggccattgacatccaccaggaatcctaaaagttcaatataattttaacttttctccctcagtctttttcaaagctgtcaataaggaccaaaacagactaatttcaaattcctcttctggttgctgtgtctctcaacagctagagctgctaggaataaaaagggagacaaaacgatccacaagctagagatggttattccccagccccacacctagtcagtcacaaaaccctagttttgatattgcttgagcagaaaccagcctccaagagaataagaagaaagggcctgggtctaaagaggaggaggaaagggttgggcacaatttcttatgcctagggatttgtcagcaactttgaggctgattatggaatattttcttgtcttccatgag

SEQ ID NO: 36 - RNA, 4396 nt, Homo sapiens
  source 1..4396: mol_type = other RNA
gtacctgacctccaaacaacggggccccaggtctgcctgccacagagggactaggggagtccctggtatctcctgagtctctcacaaactaacatttcaaactggcagttgagtaggggactaaaccaaactccctgcaccctctgggaggggctccccacagggcgctgtggctgccaactggaggaagccactcaccaaaagcttcattttccaccagatacttcctatttgatctagtagaaaaaatgtgtttaagcactaaaaaaaattaagtcatatgtgctcattatagaaaaattagaaaacacaggtaagtcagaaggaaaaaaaatcatcgcttggatataaacacagataatgtttggtttgcagccacccaaacagattatattccaaatattgtcttaaaatctgatttactgcataatttactaggaacatgcatccatgtcaataaatagacatctgcatcacttttaatatctgtatattatcccattgtttgaatttcttttttttttttttttttttttttgagacagagtctctctctgtcacccaggttggagtgcagcggtgtgatctcggctcactgcaacctctgcctcccaggttcaattcttgtgcctcagcccccccgagtagtggggattacaggcatgcaccatcatgcccgcctaatttttttggtagttttagtacagatggggttttaccatgttggccaggctggtgttgaactcctggcctcaagtgatctacccacttctgcctaccagagtgctaggattacaagcgtcagccactgctcctggcctaaagttactttaaattaactgatctcccattattcgccacttaggttttttagttttcaccattataagcaatgctatgatgtacattcaaatggaaatgtgtttacacacttattaacagtcttaattaagaagctctccatgtgctgtgtctctaacatctgcaggtatgtacacaaatacatgcacagccagcatccatcttttgcagggacattaatgatcttggctctgagcagcaccctgtcctgggagttctaaagtccagaacagattacagtgagcatctcctgggggatttagagacatcaaagaaggctgtgtccgtggttgataatgggcctcccagctgacttgccagggctgggccttagacagccctgtccaatgatttgtcaatgaataaactgttcccaaacaggctatgcagttcagtgggaaagcacaggtatgggacacggagagccccaggtggactacttgacctctctgagccttaattttatcacctgtgaattgggaataactgcttatttcataatattattatgaggatttaatgaaatcatgtgggcaaggaattatttagaattagattcaactcaagtgatgacaaccccaaactaacagcagataaaacaagacacaacttgtttctcactcatctaaaagtctacgtgggtggtgcacgatgttctattctctttctcctccacactaaacaggcctcagcctcatcagccaataaggcaggagctgccttccaggcagcggaatggaagaaggatgaagcaaaacagagggcagagtgtgcacatgtgctatgtttagggaaggttttctgaagttcccacatagtacttccacttacaaacccaacaaaaaaggctatggctaaggcagcagggaggagcaaataatgggagcaactagattttgccacagcacctatcacagtctggtttataaatggttctaggccaagaacacccgatccctgctcttttttatattctaaagcatgtatctttatatttctcaagcaatattttctctctttgaatcacagctcatctgctgcatcatagggatcccaaaagaaggacccaaggaacttgtctcagtcctctgtgccccaagaggaagctttgcttgtttgctttgctgtcaatgctgagggctcctgtggctgcctccactcaaaaccctccagcatcaggacgtcaaggctgtgatactgtaccctgagctcttggccagggcgagggaggggaggccaagcctacctacatggtgtttcatttcctaaacgaacccttacttccacgcggtctgtccagcttagaaacttattttcagtagtgttggtccttggtccctggacaaaatgtaacagccaaagtcctagaaaaaggcaagccagttcctgccattttctttcacttctgcatttcctcactattatacgtgccttccattggagcaaaactgaatgccacgcatatgcacaggagctgtgcgcgctctgtctctctcactcactctttttctctctctctctttctctctcaatctctctgtctctatctatctcttactctttatctctcactctctcactctttctcactctttctctcaatctctttctcattctctctctatctttctctctctctctctttctcacacacacacactcacaaacccacactcttattcacatctgctcaccctagccactcaaacacaatccctcattcagcctggaataagtccagagggcgtgggcctgattcagagacaatcagttgttctcatctgggaaatggggcaatgtggtcatctctagggaccctccctgctctaacattctttgaatgtggtgggtcctgaggtggaagcactctgtccctgacttctagtatatgtggagatagggttacacaaatattttattgggcagaacttttataaaacaatttatcataagctatcgcagccagcagcaatttttccaacctggattccaccaggggagcttggccggtgtctgagtgccactttcagcttgagaagcaggtgactcagtgaaaagagcaaggaggagacagaggcagattcagttcctaggccctgggccacccacctgcaagtttgcagcccagtcagtgcaagtcagctaactgttctgaacctcagtttctctgtctgtaaattaagctaaaaattcttctttcaaagagtgtcaggatgaagtgagatcgtgtatgtagggcatttaacatagtgcccgacacacagggagcattcggtaggtgccagctctcctcctggcaggagagagagaaacaaggtgaaaagagtgaattaaagaagaggaaagtcaaatgggaaaacagggggaggagatagaaagtgtatgaaaaggaaagaatggtgcgcaataacggcggtgtaatgccaccaaaatcccctcaactacttctgggcagcacccttgacagagtgaatgcttttatgagaatgtaagcggaatgtgttcccagatttgcagtaatattgccacctggtggacaaacccatgcacctttgaattttccaaaatatttcgatgaactagcttccagtcctagatgtattttgaaagtgatttgtaaattgtaaggaactattcaaattctttcattaatgtcacaaatcaactgtgtcatctgtatgccacccactattctgggtgctggggacacaacagctcacaaatcaggcaaagtccctgctctcaccaaaatgatatcctacgggggattacagatacaaatacgtaaacagatccatcgggaggaaactctcagatggaaatgagagctatgaagataacacaacagtacatgacaatacagagtgactggaaccaggaacatttctccgaggaataaaatttgaagcgagccatgagagggtctacaggtagagttcccaggcagagtgaacagccaagcacaaagctgcaccaggagagagaggtgctcgccgagagacagggaggggagtgtggcaggtgagctcagagaggggcagggccacacacatcggccacatgggccttggtagtgagtcgagatttgatcccagggtttattggagtggataagtaagcaaggtgactgaggtgctcgggtttacatttttatagttcaagctggctgctgggtggaaaacggaagttggcagaccaaggacagaatcaggcagacccatgtggaagtttctctagtggtctaggtggtggcttgggtagcgtggcagtattggagctggagaaacgcagatggattggagatttgttttggagtgacgccattctgtcttgtcaatggattggcgaaaaaagaggcatcaaagatgagttacacatcattgaagtgagaactagggagatgccagtactttatttagtattttctcagcagctcaatccataaataatttttggaagacaacaagcagtttcacaaactacttataagtcctcaagttccaaggtaattaacgtgggtgtctcattgcctcag

SEQ ID NO: 37 - RNA, 1535 nt, Homo sapiens
  source 1..1535: mol_type = other RNA
gtaagaagaaatccttttatgctttttatcctggctccctgtagaagatattaactagggacagaagataattttctctctcaatttatgtatgatcagggcagtagatttttttcttttttatctgatttgagggccccattcaacataaaaagcaattgaggcacatacaagtaaaatgtaacttaagattaattctttttttgttgtttgtttgtttgtttttacatttagggcaagcagtcttaaattttaacccacgtattattaaaagttatatcagaagaccatagaagttattcaaaaatgcagccacatattttaactagttaaaagagagagtaaaaatttggagggaggtggaggagtataggggaaaaggtagaagaaaaagagaaaataagtaagtggcaaaaaagagaaaggaaaaagatagggtgggaaagaggcagcgggacagtgtctgagtccagcacacgccagggcgagccaggtcaactgcagctgtcatattctaactgtgaattatcatctttgatcactgccctttgagatgccaatgaacttttcaagaaatatctagttctcttggctctccagctgttcttatcagccccatccaggatggaacagctttggcagcccgtatcagaacaagcagcttgacaggggcatgccatgccaggagagaggatcctaaggaagcgtggtccagtccgcacaggctctggggctttaagataaaacctcctgtctaactttagtaggactttctgttgcttcacctgccagagccctgaacgagggataaattgacttaattaactagaacacactgcaaatggtgaaagcatttagcaaaacaaagaatgccatccaagccccaaaataaaagcagaataaatagaatgcaataaacagcaaccatcccaaactgagttctcagcagcaaatctccagtatgaaattttggattttgtgcgtgtgtgcttaaaggtggatgacaatgacagttcatgggattgagctctggggtccagagttggcatctgttcatttcccattttgtcattttacccttgattgactgaatgtcagtgccttaactttgggctgtggagtgagtcggaactcccccgaggtgtgcaggtggttgttagagtctcatttttgcagggtggaagacaggagggctgcagccttcattccacactgacatggtcattgccgtgtgttctgggtccagatcaggcatattgacctgacatatgacctgacaacaggaccactcagaaagtccagcatgcgggatatgatttggagagccagtgggggaaatcataggtcctttctctgcatgtgtattcaggcaatgtcccagggctgggcggcttccgcattgcttggatatcggaaaatgcaaaaatgcccctgaagactgagacttcagtcttcaaaatgaatgtttgggaaagaaagttaacggcactgctgtacttgtggtattcattgcattattttattttggctttcag

SEQ ID NO: 38 - RNA, 1434 nt, Homo sapiens
  source 1..1434: mol_type = other RNA
gtaagaaactatttttatcagaattaaaatctcagattgattcattgttgaaataattgcacacttttaaaaggcacacctcacagccatgaggaggggctgttctgtaggtgctcaggaagtcacaagacacgtcctgaagaatatgtggctagggacatcccagactcagaagacactcagtggtgcctcttcttggaggacataagtgggggtggcattccctgatgtggcgtttcagagcattctcacccaaaaaaagcttctaaaacctccaagtatataacagtttataatactccaacaagagggccttgtagcctaaacccgggacactccttggcccattccttttaagcttcagggagtgtgggccagccccagactcaccccattcctgaggcatcctggaggttgaaatatttccagaggtttagaacctcaccaagtgggactctaggagcctgctgcctcccagcctccctcaggaactgcacctccagaacaggtgcggggctgacatgtatgtgctttcctgggcagattctagaccgtacacatgaaatctggctttcaggattgctctccagagggacctgtggggcctcggctgagacagagagtaggagtgaggcagtgattcaaggccctgagaaagagctcctcctctgcttggtataaccagctaattcattctgttctgttgactttggcttctgccctgcctttgaagggtttgaggccagggagtgatgcactcagactggtgtttccacacagtcacttcagacttccagggcagtacaggagatagatcccagggccagtgaagaagcagagcacaagtccaggcaggagaggctaagggcctccctgaacaggtgtgaggcacagaagccccgagaggtagggatgacaggatgaagatgggtcctgtgctgctagaagtacctgcaaagcacagaggtggcacagaaaaggagtccttggctgggatgggaggagatgacatgtgacatgtgaaagaggacctggagttggctcgatgctcccaaaagggaaaggtgccgaggggagctagcagccatgcaaaggcagagacatgcaggcagtctgggccatgaggagctctggaagtgactcgatatgtccagaataggccactccagggaagggctgaggaaggatgaagttggagaggggcacagaccagatgcagaagggcctcagaggccaggatgagggtttggactccttcctggaggcagcagcagtgggaaaagagttaaaagctggtttgtaaagtggagccatgttgctcgctggtccaggcaattcccccgaaagttcatgtttccctacaaaacccgagagagctactagtaggcgtgaagttcgtggccctggtctgaggatttcctgtttccttgtcag

SEQ ID NO: 39 - RNA, 131 nt, Homo sapiens
  source 1..131: mol_type = other RNA
gtatgtaaacagactggagatttgagtaggatttttgacttgcttaactaccatgaatgagaaactctcatgagtgataacaggaaaaaaaaattaaaaccgtcttgtttgtttgtttacatggtttttag

SEQ ID NO: 40 - RNA, 230 nt, Homo sapiens
  source 1..230: mol_type = other RNA
gtacttgacctatgtataatctgctctggagctaaaaatttacctgagctggttattttatttttactttcctaccttcattaaattccatccctcctcctgctgaaatctagcaaggaatgtcttccagctaccaaacccttcctgcttctcaaatttcctttccttcactgatttctgctttaactagctgttagtgcagcgtctcagatgtcctctccaccctctag

SEQ ID NO: 41 - RNA, 1480 nt, Homo sapiens
  source 1..1480: mol_type = other RNA
gtaagccaccacagccccagcctcaccactttcttgtcaccttctccactctttgaacatcctgagaggattctcaccaccgcgaagtgctgatttggatggtaatgctgtttagtcaggcacatatgaacatccgactttcaaataagtgcctcacacttcacataccagacctcttggtcattctttctccccaacatttatgtggcaagtaagtttacatttggttccattcccttttggcttttgatagcaagttgctcctggagcttatacaattattatctttgctatgtgcaaagcagctgccaggaactggcaaagttcagtaaacctttcagctccctcggagtaattatcttagattccaggaatttcctcagaagagcatactttggagatgtcgacagagctttgctaccctcaagctgaggctcttcttgcacagtttcagccagtggagacagtggccttgtgcgttttgtagtatgttcactctatttgaggcctacatggaggaggggttggtaggagcacctttgttagtgcaaacttcagcaacgttgtggggtcctgattttactatcctagcacacgctgagtgccagtgaacatgcccagggtcatccactaaaacctgggccttggctccttggtgtcttcctctggacaccctagggccctagactgtcctctgttaattctcactcagccacactttcgtgtgtctccttccagtcatttgttctaagcttactacgtgtatggatgatatgatctgtagttttatcaaggtagtgactaccacataggatacctttgtggaaattagtaaaaatgctcttttctgcaggtggacactgtcccatgccaggggttatggcttgtacataaagttcaggctggctttagccccaacttacccctcagccagatgccttctatttgtccgaggaaagaataaatagagccaagtccctgtacaacttgcctgccctcttttcacttaaatttacatcatgaacatttccttgtgttacgatgtacttcttgaaaatgtgatttaacaagatgattattaacaaaagataaatctcacagaccgtatgtctgtcaacatagaaaattcaagagactctatagacagattattagagctaatgagagcattgcagtacataagattaatataaacatctatttctatacaccataaaaataattagagaatataataaaaagaaaggttgtctagaaatattcacatgaaatagaaaggcaacccgcaaatacccatttaaccttggtccatatggattaagacagtttagtggagtgacagcttcaaggtagagaagaggaacctggaggccacacctgggcgggtgtaaggccttcccaaagcctgactttgtatcttctcctccttctgctcttccctcttcatcgccctctccctgtgtctctggccctgctgcag

SEQ ID NO: 42 - RNA, 3727 nt, Homo sapiens
  source 1..3727: mol_type = other RNA
gtaagtgtcagtttacagcgcctccctcccctccgtgggcccaaggtggagcttgtgtgtgctctgaaggaccagaccaagaggggaggggttctcacggtgccagggctgctgaaaggcactgggccaagggccttgtgtatctgctgtcccttgacatcttctcagaaaggcacagaactaggagcccgaagctaggaaaggctgtggggtgcagcttaacaactggtgaacgggggctctctatgtcctgcactgaggggtcttctgacccatcaaataatcactgcaccgcaggcatgagtctggccttcctggcatcagtctggcgctgagaaggtaatatgaaggggtctttcaccccaagtccccttctcaaatcctgccccaccttcaaaagggtaaaggtaaaactttccctgtggtagggtcaccagataaatacaggacacccagttaaatttaatttcagatgatgaataatttttagtataagcatatgctacttcaaatattgcacaggacatatctacactaaaaaaaaaaaaaaaaaaaaaaaaaacctggttgtttatctgaaactcaaatttcactaggcatcctagatttttatttgccaaatctggcaaccccagccagtggccaaaataataagaccttcacttattagattaaccaccgctacagggaaaaatgaagaaaaaatatttattaaatcaatagcacactaccaccttcctgacaaccaaggttggtgggggtagggaggggtcaggatagcgtaccctattacaggctgcagggtcaaaggaattggtagtaaaggcctagttataatgtaacagggatcattatgacatcaaccccaatttattctaggtgtcttgagtagtaaaatctcaacattttaagaccaacatgagcctccatttcatgtgatgataagatataccaactgatggagaccaacacaaatgaccttctcatccatggttttttaaaatgatggtgaatattggaattcctgaagatatgatttctatcttactcagcttagtaagcagctatcacttaacaatacaaaaccagagattatcagtagcaactaaattatttcctctctcttctgtctacacgaggaaacactcataaatgcacggggaggaggtcagaacctgaaagcctttctttggataagagcatcaactgcaggtaccacattggccctgtgatgctaatataaaaggagctaggcccaccggtaccgaaaagttacttagaaaagtgcggaggcttttaattttactttttttaaaagataagaaatagaatttacacacttggggctggcccacgtgtttctgtgtgtgtgtatgtgtgcacgcacgcgcgtgtgcgcttacagggatctctgagcctatggagagagatgtagctaggatagagtggacatctgaggtgggaggtgatactagctggcagtccaatgaaggggtagaagatggtaggcatcatgttagcaggctttctgatgctccagaattttaaagctggcctggaatctcacctccgcgatccatcattttggaacttaggaccaccattagccagtggcaaaaaaaaagttgaatgaaggaacaaacaattattgcttatgtaattcacttagcacatatatgatgttttaaattcttatatgtgtcatctatttttctttactttaaaattttgcaacagttacagacttatggaaaagtcacaagtacagttgaaacctttttttcttagtcatttgaaagtaacttctcagcaagatgccccttctcatttatttctctcttcctgtctctctctctcacacccctcagcacgtccgatgtatacttcctacaaacgaggatacaccccatacaaccacaacacaaactgtcaacatgaggaaaccagcactgatgtgtcatcaccacctaatcctcacaccccactcctctttcgcccattgccccagtgatgtctttcagaaaaaaggatctagctcagaatcatgcatgacatttgattgtgctgtttctttagtctcgttcagcctggaagagttccacagtcttttgttaacactcatggtcttgacactttgaggactgcaggctggttattttgcagaatgtcccttggtctgagcttgtctgaggtttcctcttgcccaggttgagggtgtgcatcttggcagcagtatcagcaaacagatgctgtgttctcactgcatcctatcaggtggcttctgatttcaatttgctctgttactgatgatgttcaattcggtcacttaagaaggtgtctgctgagcttcttcactgtaaaattactcttttcccctttataataaatacaaatttcaggtagaggcacttcaaagatatataaatatcctattcattatacaattttccatttattcatccatttatttatctctgtatgcagtcatggttcatgtgttaatcaatggactatgatccaagactatcattatttattttgatattcacattatccccactgtggtcagtggggggccgttgaagctggcttctgtatcgtcttgacttgggtcctcatgcccctggacctcctccatgctcaatggcacagcaagatattccaggctcatccttccattatccccattcctaccctctccccaagaagccctggttcctgccagtgggaagtggccctcagaagccaaggtctgagtgctagatatgttcattgcctctggagcaccattggtcccaggccttctcagtgatagaactagggaagatatggatgtacacacacaggtatgcacacacctctatctatagttctctatctacctatacagtgaacactatgagctctccaaaaccaactccacagggctcattctagttttttttctttccacatctgtaactcccttctccaacagtgagacgctggcttctctcactcccaactcatttatctaccggacctatacacctgaacagtgcccaactctgccaccatcccctccccatgtggatgccgtcctctccctgctccagctgcctctgctgcatgcaggtcctcctcgttctgctctggctctgataccctgcaccagatcagcctcctgtaaggatatctttctcatcccgttgaggcctccacaccccacggcaggttgccccctgaggaagcccgtctctggttcttgccctgctcctgatcaccatggctcctcccctaaccccactgttgccgtcccctttctgtgcccagtatagtggctgtaggactaaattgtttaaaaagggtatcattatttatttgagctttgtgaagccaagaactaggctttaagtttttctgaattctgaagacatgcttagaaagaagaatcaacaaaactttatgaccaaatagaaagagtgagagaccaggcagaattttgtaattgatcctttcaaaagatacaaactaaaggttcccttggcagggaggtagggcatggggtggggtaggaggactagtgacagcttaacatatgtttgccaaccaagaactgtttaaaaagcaagtcgaatcagaatcccagaccctacgagctggaggagcctggccccacccctcattttgcagagctggcagcaggtctgagaggttaagtgacttgctctcctcttctctttccgag

SEQ ID NO: 43 - RNA, 1048 nt, Homo sapiens
  source 1..1048: mol_type = other RNA
gtaagccgtttgggccattagctaatgcctctgaagagaagcctggtggtgggggtgggggatcatctcctgacagaaaacctgggctgtcctgtggtggtagcacccacaagtttagcttccggccccaggtagggtctgaagctgataaccagggatctgtctggcttctgattctgactccactgacagaggtatctctgaggcctggtcctgtcagtgacaatgagagaagtcccacatgatctgaatctcctactcaaactgaggccttgaccaaagcctgggggcagccattccccaacccctcacccagctctgactctcactcatctgtggccaatctgtccacctcagtgtccccatgtgaactggccaagagttaccgcccacagtagaagactccggccaaaaagctcctcctgagtcagggacagaggatgacacaggggttacatcagcagagttacagggcccagcatgcaactttctttcccacgtgtgtaaatttgaatgagtaattcatccatctcggcctcagtttcctcatctgtaaaagaaaatagtgatcctggtccttcctctgtgggccagtagagccttgccaaagcattgttctccacatctttctcttggaaatagagaatttgggaaccaacctgactataagctgtgaagatgagctcactgggctcatctgagatgacctcagctgggctttgctgacccaggctagagtgggaggtgttgcaggctggagaaccctcctatgaattgtacagggctttgtagtttacagagtatatacacagctagcagcccatttgctcctcacaaaaccccatgaagtggtcaaggcaggcatcattatctccatttaaagttgaggcacagagaccaacaaatggagtatctctctggtcccctgggactctggccagttcacacacatcacctcaggtgtaaggggagtgcattatatccagacgtattgtaggtggaatggaatgtggaactccatcactctgagttgtctcatttcacacag

SEQ ID NO: 44 - RNA, 3157 nt, Homo sapiens
  source 1..3157: mol_type = other RNA
gtgagcataactttcttggcttttttgtttgattagtaggatagtagagtatgtgttggtcgagcagagccaggggcaagcatcgtacatgtagcagctgtatgcggatgagtgccactttcttcctccctacccccgaccctgcctcctttccttccttccttcctcccatccttccttcctctttccttcttctcctccctcctccctccttcccccgtccctccttccttcctttttcattgcttccttccttccttcgtccctccttcccttcctctttccttctgccctctctccctttttcctttcatcctccctccatccctccctccatccttccttctttcttccttctttccttcctataagcacctttttcatttctgtgctctgaatgaaatggttttctgtgtttattctgcaagcaaaacttgattcttgcaataaactttaagctttgcttactctttcagaaaggttttctcagggactttgggtgttgggttttacacacacacacatcaatacatttgggtaatttcaaaatctaaaaggaacaaaaaggcatacaatgaaaaaatctccttcctacccctgtttcccactcatgcagttctcttctccagaggcaaactcttacttgagtttcctgtgtgctctggagacacatcagcagatccctatacggtctttctcccgctttcttatggaaattgtaacactctgacatatactattccttgggcaagttaatcttgatgaagagactgggtgttctccatgctgaatgcctcacttttatgagctgccaagcccagttgtcccttccacctgacctccccctgtccagagacagatggccaaactgaatcataaaaagagggggaaaaaaagaaggcagtcgctgcagggctgtctttactccacactccacactcccagtccccaccgctgtgtctgagtcctggctgtggctgtccttggaacatttgcctcaccacgtgcctgtgtccccaggcgcctcaacctttcctctcctcattagctcttcccagttcagagggtgggaccggccagcacatctgcactgctgccctgccacacccacctccacctgcctctgggccccactggggaacacaggacaaatctgtgcggaggccccaccatgaaccgcccagacccgtggacccctgagactgactctttccagatcttgttagggtttcgtggctgctaggcaagtaacgaagcctcatctgtcccatgaatgataagaaattcagcatgtcagagtcagactctggaaaggcggggggataagaacacagccccagcagatggccagagcacccaggtgactgaaagtgctgctttgcagagctgtgtttgccacaggctcacagcccactaagtcttaagacagttttccttcagaataattaaatagccagcttaaagcaactcagaacattttcccctctgaggctgcacccatttagccaacatttgctaagcacccgccttcaaaaacctggtattttcatgtaaattatccgatacacagctgctatggaaacccccagtatcccacaggaagctccccagctcccagcagctgccggcccgtgtgagatcaggaggtctttaccagctgaacaccacgtgccgggtgtgtgctgatataaacaagcgtggcccactcgtcctgccctccagaggctcccgttccagtcggaaaaggacctgcccacgaagtttgcaacgatataagccacagtgtatgatcctccataatacagcgtgtgacagagcagcagaggagcgaggcagataacatgctgcaggccagaggcagcgggaagagccaggctgcaggggctgggggagccgtggtggaggaagttcaatttcagcctgtagatttctattagcccatttaataaataatgaagtgcctactctgagctaatcattgtgcaggtatttaggaaggacaaaaaaataattaggactcagtgcccaccctccaggggcccactgactagtagagaaagtaggcagatttttaaaaaattaatcatgggaatgtgataagtgctgggagagaggaatggatactttctcatgggaatcttggaaggcttgtaagggaaggcactctctgagccagctgtctaaagaagaacaggaatctttaagaaagcagaagggaaaagagcattctttcctgcttggagcaataggtaacagcctgcacatgcccaggcctagaggccaaagagcacagtgattccagaaagagtggggagaaagggtaggcagggaaggatgaggtaatgtgggcgcaggtgtggaggctggagagggaggaggttgtgggactgggaggagccagatggaatggacagcagtggcccagccaggagctatgctggcctcgtacgcctcgatgtcccttctattttctcaggggaggctctgcccaacatgccaagtccgaccacttgaaaacaagtccctggcttaacacagaccccagagagagtctccaaccctcctctccctagacaatggtagttgccctgtgaggggctgaaaagcagagctggagatggctcagggcctggtgttaacaaatgccttgagggctcctgttgtttcaaagtgagtctgcagggagagctccctaagtggacagcaggagggctgcagcttctctgcacattcctgctgtcacccccagagtcacctaggggaggggtaaggacagtaatgcaggttcctcacagttagcctcggtgcccacatggtactgagcatagtaaatgtttagaagatgctgcctggctagacaaaggggaagctcccgcccactagaaacttgcagggagccccagtccttgattggtcatttaattgattagctccttggcctggccttgaggcactgcttgtaagtacttcatgacctccattgcaaacccatgatgctctgctggacaaatccctccagtggccagtctggctgcaaggactctctgtctgcaggccttgccctgtgctgtcctgtgagagcatctgggccccacctgctgaagagagggggggtggggtttgccccgtttccaacagtcctacttctctgtttcag

SEQ ID NO: 45 - RNA, 332 nt, Homo sapiens
  source 1..332: mol_type = other RNA
gtgggtggtagccgaggcccatggagcatgggccctgggtccaaagctgggagggttaccgggggggctcctgcatcagactgtggcaggggctggtgctaggaggggaccttgttgggctggaggtgtcctgccagctggagaggattagggtgcctctgtttccatggctggggagccacaggagggatggagggcagcccttatgaggcgggtgtttggctcttgctcagttcccacataaggcctggtctagtgggccctgtgctgtggccaggtctgtggggtgagctggggcggctgaagtggactcaattcctgttgatgcccag

SEQ ID NO: 46 - RNA, 1928 nt, Homo sapiens
  source 1..1928: mol_type = other RNA
gtacgtccatgccacaccctgggccagtgggcagctcagggcatccagaactggaccttatacccacatggtcatttctttcctcaggagccccactccacaatgttttttctacattctcaaagcctggcttttctccaataatacaagtagaggatcgggttaaaataggcacattcaaatatgtgaagagcatccactttaaaatatttaaaatgcagtgctattaatttcaattgctgatatttaatccttctcatttaattaccaaatgtgtattttgattagatgatagtattgcaaataacaatggttacagggtatccaaagtactaggaaatagactaatgtatttatgagagaaaggacacagcaggcccctttgctaattagagatttgggagcatgggagtaatatgggagccatgtggaggggtgcgggcagtgatcacgaccccccactcctggaggaaggtgggtagctgccaaccctgacttttgaccagggcttctcaaatgccaggttagctggcaattgccattcttccgcaggctcttcctgaagctgggtgggcccctgcctcactcccctctgcaatccagtcctacctttattgtcctcacccaggggcctgaattgccaagcagcagcccttcctagcaagctttccccaatagtgttttgtttcttaacttttcctcctctcaggctgagtgtggtcacctgtaaatagattccaaggacttggttttatgttttgatccacagggaattgatttattggaaatgaatctgcctttctactcacaggactgtgagaggtgaatgagatcacaggtgtcaacacacgcctgatgaaacaggatacacaagcagttctagttatgggagacagtgtcaggaattgttgtccttggcaccctcagcccctgcagaccctttctgcagccttggccataccttttagaggcttttgtgtgggagagagcaggtcaggaggttgactacccaaattgactcattagcttcaaactctgatgtcaacacatttgaatgagtcctgcctgctttagggcctaaagaggaccagagaagtacaccatagtccctggcttccagaaggtcagggagggtttcaaagaagaggctgtgtctttaagaatggggaagattccatttggtggggcaggaggaggagaacattgagggactggaaacacatgcggaggctgggagacgggaatgaccaataggactgggaaccagggggagatgccaattgctgacagaggagttagtgcaagaggtaagtgagaagggtaggtggggctggattgcagggctgtaactacagctgcagagggagggcttcaacctacagctgatggggaacaacagaaggttttgaggcatgaggtggcctgatgacaactctgttttggaaaggtggagttggcagggcagactggaggaagtgggaggctcggaggttagtaactaccccttactgagtgcttgctgtagaggaagcattttagtcctgacggtgatcccaggccctgagtctttactctgtgccaggcactgtgctgagttcatcttcagcacaatcctatgagacaggtattgttaccctcctcctcatcacatggttgaagtaggcaaggttcagagaggtccaatgcccaagatcacacatgaggaggccaggactggaacccaaggctgactctggacatgagcacctgacctctctacctaatgcctaatgcctctcctgctgggagccctttttagaatttaagtcttaaaggatggaagcccagaaggaagcagaagcaaggaagtggaagagaggtcccatggaaaggacagtgccaaggacactgtacagccagcccaatcctgaccccttttcttcatctag

SEQ ID NO: 47 - RNA, 453 nt, Homo sapiens
  source 1..453: mol_type = other RNA
gtaagggaatgggtatgagtttggaggtgctggttagatccacagttggcatgatgttgccattttccttctatagaacaattgatatgcttatgcaagcaatttggttcccagttttatgtagggtcatcatccctgtgttataactcgtcttccaagagcatctaattccaatgtgtgttccctgctattcatctcgggcactgacacagggcctcagtgagaatcactccagctgagcatcattcccttttctgtgttctgtttctgcagagcatgggtcagcctcgagatgtctcagtactcaccacacctctgtgcctgcccatgtcaatatgtaacctcctagtgctggtagttttctcctaaaccatcctttgctctttgttccctcttcccctccttgctctcaccctgtctcagttctcagtccggtttcttcgtatcttgcag

SEQ ID NO: 48 - RNA, 494 nt, Homo sapiens
  source 1..494: mol_type = other RNA
gtgggtactctgcagaccacgtgtgaaaggcttccgaacatcagctcttgtgcctgcctctcctccccataaggcagagctattcaataggaacataatgccataatgcaagtcacatatgtaattttaaatcttccactagccacatgagaaaagtaaaaagaaaataggtaaaattaatttcattagtattttttattttactcaatataaccaaaatattatttcaaaatgtaattaatagaaaaccttattaatgaaatatttgacaatttctcgttgtttttaagtctttgaatctttacactcagggcccgtgtcaactgggacttagatgtgtttcaagtgcttagtagccacatatggctcgtggcctctgatggcagcccaggtctaaaattcctcccccagctcacacacacacttaccctggggcctgacattttagaccttcttgatctctagggccaggctagctctgtgttttctcctag

SEQ ID NO: 49 - RNA, 2051 nt, Homo sapiens
  source 1..2051: mol_type = other RNA
gtgagtatcctgctcctcctgtctcagggagtctctcacaggtcctgtgagaagaataggaagggtgatcatcagaccctatagtagggtggctctgaggccctgaaagatctgtacagagaaggaggcctcccagagagcatggcccaaaaagcccaacacatagacccaatggaaaagtgaactgaattgtgatagttaagagattcctctgttgggatggattcttggaaagacctgggaagcactaagtgtgtggttcttaatctcttagaggtcacggaaccttttaagcatctgatgaatatttgtagcctattcctataaaaatgcaccattgcttcccattacctccctccacacatttttacaaaacgtttcagggagtttactgagccccaggtcacatttatgatcctgcaggagctcttgaatcccaggttaagaacccctgtgatgaatgaagaatccttcctctgggttgagtttctagataggggctcatgcatgggcctttggggtagcctaacctgcattggctatttgtaggctgatatttggctttgccagaccaaggagcatagagggaaaactggcgtgtgcccttggattctggagggtgactgctgctctctgtaataaaatgtgtttaaacagactggtcccctatgggcaggacagagaggatgagctctcactcatctgcctctttcctggctgcaggaaaagcttgaacagtaaaacttcagcacacacaatagaggtgcccagaggaagcctctgccctggtttataagtggagttaggtgctgctgacatctgtccagcatctgcttgactggggcctcttcctctctcctgaaagccatcctcagcatggcccaatgcccagtgggcaggacgagtcctgagcacgcttcactggctcagacaggatgaatttgattctttggcctccatagccagccctactgggtttacagaaaagggacaggcaggggtgaagccaggtcatggctgagtccatctcaacagatccagcttcacctgcaagtgaccacgcaggtgacttcctcatggtgacaaaaggagtcatggcagggtagagatatcataccatggcaggggaaagatatcatagaattttccatgagcacatttatgagacatcaagttacaactgtgtccaagtgaggcacagtctgacatccagaaggtaaaactgagctggacgctagaaagaaactataggcttaagacacagaattgggattatatggtagggtagctcccactaatttggaaacgtaccctacttgcttccctgagtagttttaattggcccagccatgcctttggtggcttttgtcattgtggggaactgtaatggtctctctgtaccatcctatatcatccatcctttattcatagaccctaagctataagaagaaaaggatgagattagactaaatgtctatgtatagtttattttccatcttggcaatatattttttagtgggggtgaatatattagccaaagggagttggtggaacccaactcactctacccctgctccctgcaggcctctcgctgtgggtagttatctgactggctcctctttcattgctatctttgccaataaatacagatagagaagtttacttccatcgggacacatgcatcttttctagttacttcccaaatgtctgaaaattattgataaatcatgaatcattttcttaaacctgatcttccctctgtttttaaactcacatgtgaggtgatctgatccaaaatgaaagctgacttttggcgtaacagggattcaattaatcctagacatggaaacatggaagaatctgacaggattcagtttctaaccgaagggcccctgttttgattcccaaatatcccatgcatttctgaagccaaataggagaagagaagaagcagcttccttttcccgttggcagaagcttctccagccctagctctatggtcatccctccactccttgaaggatactcagtaattgctttttttcttgcag

SEQ ID NO: 50 - RNA, 3448 nt, Homo sapiens
  source 1..3448: mol_type = other RNA
gtgaaaaatgttttgttgtggccacataggagtctggttaattacaagcctgtttcatgagagtgcattctcttggagatgagaaactgaagcgtgctattcattcattcattccaacaaatgtttactatgtgtctactgtgtgccaagtactgttctagaaaccaggagtatagcagtgaacaagacagacaaaaaaaaatccccactctcatatctaacaaaatgttgtatgcatttatcctctgactcagcaatcacacgtctaagagtttatcctgaagatgcatctcccacagtgcaaaatgaatatgtataaggtgatccattgcatttgtaattgcaaaatgctggaagttacctaaatgtttagtcattgtagattggctgaataatttatggtacagacacacaataaagtcttacgcaactataaaaaagaagaagaaaagtctcagtaaactgatatggagatatttccagtaaatactgttaaatgataaaaagcaaagtggaaaacagaacatagagaacgctactttgtatgtaagaaagaaggaaaaacaagaaagtaaacgtatgtctgcttacctttgcaaatagaacgtagaaaggataaaccagaaaacaatgaatttggtgatcaacaagaagaaaatgggaagaaagaaaaatgggaggaaacagtacttctggggatatatttttgtatagttttaatttttggaagcatgttaatgttccacatattcaaaaaaaatcagtaagaatgggaagtaggcaaaaatgaaaacaaaaagaaaacctaacactgacagcaaactaaataaagtaacccaattttatttcaaataaatatcataatcttgcaaaagggggatagagctaacacaaacaactgctgaacacagtgtttgactctatatcctcattcttgggcagggtggagcgggggagaagaactacaaataatttctgagttctttttagtttgttttttatagtggtataggcaaagtgattctgaaaattttagatgtgttacaggattaaataaattaataaatgttttgatgttattgggacccagaattctcaccgtggaagaagggacttacaaatatggaaaagggaaaagcaagaaagaactgtgaggtcatggataggaaccggaggtagcactgggaattcaggaatatttatatgcttgtgtttgtgggtgcatgcagatgtgttcatgtttcatgcacataggcatgtatatatagacatatatttgcatgtgtgtatctgtcttccgaaaggctcaagaagcaaaaacaccccagtagccatgagcacacttagcactcaggcttttgtcttaataacattccccactaaaagtaaccctgattcctccaataaatgataagttccagggctggaatggcataggtataaaatgaacctggaatatcttatgccagaaagtaaggaagtgcttttaaaaaaaaaataaggggctgggcatggtggctcacacctgtaatcgcagcactttgggaggccaaggtaggaagatcgcttgagcccaggagttccagattagcctgtgcaacatagggagaccctgtctctacaaaaaattagcaaacaaattagctgggcctggtggtgcacgcctatagtcccagctactcaggtggctgaggtgggaggaatgcttgagcccaggaggttgaggctgcagtgagctgtgatcaagccactgctctccagcctgggaaacagagcaagactctgtctcttaaaataataataatataattttaaagaaataaaagtaactctgtacagattgcttattggttacatgggagaaacataataattttacaatggagaaattagacagcaccttaactgggtgatcaaaattaaccataaggggcagatggacatctcatgccccgagatgtgataccctgtgaaggacacaatttcacttatgtagaatccagattggagatatgtaacctgaatcttatcatgaggaaacatctgacaagctccaaagaaggaatattccttaaaaaaaaaaaaaggagactgtattcttcaaaaacataagagtcataaaagacaaagaaagagctatggaaatatctctgatcgcaggaggctaaacaggcataatgactgaatagcagacaatagactacatcttgtgcagaagagaaaaaaaatgatagaaggatattattggaccaactgacaaaactgaactatgaacagtagattaggtaaatgtatcataacattaagtttactgacattgataatgtactgtggttatgtaagagaagatctctattcttaggaaatatgccctgaagtatttaggagtgaagggctgtgatgagtaatttaccctcaaatgggtcacaaaaaattgtgtgtgagagagagaagggttttattagttaataattctatgaactatttttattcctatatgtttgtgtgagtttgaaactatttccaaataaaaagttaaaaatggagattacattctagtgggagggatagacgatctgtagataaataggtaaaatatccagtacattagagagtgaaaagtcctcagggaaaagtaacgcagggaggaactgctggggcagggtttgcattttgaggtagggtggcccagggagagcctgcagaggagagaacctgaatgaagaactagaggtgagagaaggagccacgtgcacacctagggaggaacattccaggcacgggggactagtatagaaggcagaagcatggtgagcttgtctccagtggcttccctagatcccctcctgcgcatgtgcacacacacctggtgtctctgtcatcgttccctcacagcactgtcacgatctgccagtattctgtttattttgactgccacctccccgcagtctgaggatagcagcaatggctgtgttcacattgttctccagtgcctggttcagtgcctggcgtatggtcagtgctccataggtatgtgtcggatgcacaaggctttgggtgtaaccctcttgacgggtgggatcaacaggtctgggactcaccatcttctcaaacagagccttcctcctccactgctagccatggtccaggacgctgggcgagacccactgtcttgctctttgtaaggctgaagtccatttcccaggcggctacacccaacagatgctgagcaggctgggccaccctgggatccaagacacagagagaaagagcccctgtctggcgcctgaagcacatgccagaggacaggagccagcaggagcctgtttcagcctagctggggatttcattctggaggcgtgagatctgggagcccaaggctttgaactgggggaggtttggggtgtttgcttgtcttctccaaatggcatttctttctcttccctag

SEQ ID NO: 51 - RNA, 752 nt, Homo sapiens
  source 1..752: mol_type = other RNA
gtaactgcgggcttgggccgcaccaagggcttaaaccaagtgctgggtctcttgggttggggaaataggttctgggtcggcagatttagaaactgcagcagtttggctttagtctggactgtttcctgtgttgctcattttgagcgatcagcccagtgtttggttcacacagctccggagaaaaacaagtcacggcacagccttgacttgggactgcgcacatcctgcgttcccaggatgtctcctgtggggccatcggctcacagccgggaagttcagcccactctgcggcctgtcggtgtctggtccccatacaggagcactgagctgggtcaaaggctcctgagctgagccaggccaggcctgaggccatgcccacgcagcccaaggatcatgagggcacaggacatagcgggaaccaaggaagtgacctgagtgacctccctgccttctgacaaatgtatttgcaggattttctttttttgaggagaattctgtcattgccttaatccactttaatcccctcgtgggctgaaatgggcccaggatggacgccacgcttctttactcttggatccacctcctgccttccctaccctacaccagggtacccctgtcttgctcaagtgaggggagtgactgtgtgcgccttctgtcagctcatcctccacaggggagccagcccagggggaagcagtaatcagaagggccagctcccagcctgtgcccccaaccttctctccaccccccag

SEQ ID NO: 52 - RNA, 73 nt, Homo sapiens
  source 1..73: mol_type = other RNA
gcaagagattcccagggctggggaaggtgggtgggaatcctctcctgctcacctcctctctcctgccccacag

SEQ ID NO: 53 - RNA, 2725 nt, Homo sapiens
  source 1..2725: mol_type = other RNA
gtaagcagatggtggggcgtgccccttgttgccttctgtggatccacctggatcctgtgttctccattgacacttggaagagtcctgctgctccgtcatcccctggggcagaggcaggtggtggctgggcctcattctccagcagcagatggagaaggccatcatgctgataagaaactcctctatattggcctaatttcctgtggtcgaagactcgcccaagtctctggatggggcatctgatcaggatgcatgcagagcctggctgggatgagggagggctgctaccactgcctcaatatttcaccacttatctcaacagatccgggacctgtggcctatttactaagagtccactccaatgtaggaatggttaggagaccaactgacttgaggacccatctttgtttttagaatattgtatgcttttgagtttgaaaaaagaccatatgttatatgacaaaccaacaatggcagtaatcttgaataggattatccttatcctgtacccacacattgtaaactattgtagataattccttattattaagagtttgcatgccaaagctaacagtttaagattatcagcatattgccgtgctcattcacgttctgatatgctttataacctagaaaagagcagagttacaattactcatttatttaacaaacacttattaagagctcagaatataagtcactaagctggttggtgggaggaacagcacataacccaccttatctatgctgaggtgcataatcctgatgcacccacaggagggtgttacacagaagatgtcatcctttcatatgtgtcagagcagataaataattgagagaaaggtctaatagattagctgcttgtggcaagtggacgtttgacccatgatttattgagcaactacaacttggacactgcatagatatctatagaaatagcagcatgtcaggtcaccagacctgtgtcagcaacttcctgtgtccaactgctggagaaagggaagtctcctattcctttccctccagctccttaatatctccatgatagagggggtgagaggggagtgttccctgtgtggagggatggtgagttttctggagctgaaaggtaaacagcctttctcctctgcatcttactgcagaggagaacagccctagactgtggaggaagctttggagtcagttatgactgacacaggataccagggcatagggtactgacacccgctagccgtgcacacactctctggtggaccatcactcatccaagagagggtaaccagccatcctgctgaaggagaaagaaagcaccaatggcccaagccctagcagctccattgtttcaggaagcttcctcagggaagtgctgccttcccgagcctttgctcccacctggcccatcagcccttaccaccactcagtatgcactggtccacgtgtctttatgggcagtcttgggatccccacactgggctaaaactacctttgacggccaggtgcagtggcttacacctgtaatcctatcactttgggaagctgaggcaggtggatcacttgaggtcaggagttcgagaccagcctggccaacacggtgaaaccctgtctctactaaaaatacaaaaattagatgggcatggtggtatgcacctgtaatcccacctactcgggaaactgaggcacaagaattgcttgaactcagaaggcagaggttgcagtgaatcgagatcacaccactgcactccagcctgggtgaaacagcaagactctgtctcaaaaaataaaataggctgggcgtggtggctcatgcctgtaatcccagcactttgggaggccaaggcgggcggatcacttgaggtcaggagtttaagaccagcctggccaacatagtgaaaccctgtctctactaaaaatacaaaaaaaaaaaaaaaaattagccgagtgtggtggcaggtgcctgtagttccagcctctcaggagactgaggcaggagaattgcttgaacccaggaggcggaggttgcagtgagccaagatcatgccactgtactccagcctgggcaacggtgagactgtctcaaataaaataaaataaaataaaataaaataaaataaaataaaataaataaaataaaataaataaaactacctttgacttcagcaagtacgattatcccacattaccatgcagacatttgatctctaaaaactggtatcaaatgatttctccagggactaccatggtttttctctcctagttttcagtatgtacacaggtctatggtatgggcctttaatccccagtatttctttttttgttgttcttgtttgggtttgtttcttgtttttcggtttttttgagacagggtctcactctgtcacccaggctggagtgcagtggcatgatcatggctcactgtagccttgacctcctatgctcaagtgatcctcccgcctcagcctcccaagtagctgggaccacaggcatgtgccaccatgccctgctaattttcgtagagacagggtctttcttgttgcccaggcttatcttacattcctgagctcaagtgatcctcccacctctacctcccaaattgctgggatttcaggtgtgagccaccaagctgagcttaatccccaaaatttctgatgagtctactccttattttgggattaccttaggcccaaccactaacagaggcctgtcctgcactgtgtgcatcccctag

SEQ ID NO: 54 - RNA, 1665 nt, Homo sapiens
  source 1..1665: mol_type = other RNA
gcaagttggccctggggcaccgagagctgagcaaagactggtccagaacacccagtgtgggttggaattgccataagagggaggcataacattcccgatttttaacaaactcttgccctctgtttattggggtaaaagctgatatatcagaaattgttttctaacaatattttttagtcatcaggaaacttcattgattcttttttttacattttccttccctgtgatgctatggtgtgttatttcattcttgctcgtttgtggtggtggtttttccttcaaatcagctttattgatgtgtaattaacatacgatgaaacacaggttctttgggaggccaaggcaggaggatcacttgagcccaggagtttaagacaggcccatgtaacaaagtgagactttgtctctacagaaaaaaaaaaaaaaaatcagaaaattagccaggcgtggtggtgcatgcctgtggtcccatctacatgggaggttgaggaaggaagattgctggagcccaggaggtcaaggctgcaatgagctgtgttcataccactgcactctagtctgggtgacagagcaagcccctgtctcaaaaaagcaaaacaaaacaaaaacacctattttaaatgtacagtttagtgagttttgataaacgtgcattccatgtgtggtttttaaaaatgtaatcacattttttattgcggtaaaatataataacataaaattgaccatgccaaccatgtttaagtgcacagtgcagtggcactaagtacatttacattgttgtgcaaccgttaccaccatccccgatagaactctttcatcttgcttcagtgaaaatctgtgcccattaaacactaactcaccacttactgcccccctcgcccttggcaactactgttctactttctgtctctaaggctctgactactatagatacctcatataagtggaatcatacagtgtttgtccttttgtgtctggcttattatgcgaggacttagcataatgtcctcaaggttcatccgtgttgtatcatgtgccagaatttccttcctttttcaggccgaataatattcctttgtacgtatatgtgctacattttgttcatccatctattcattcattgatagacatttgggttgtttctgggttttgtgtttttatatatgtttttttaaaaataaacatctttagagacagttcagtaaagcagtggaaacagggaagtctccatttaacccctgaggatctggctcacctgcaccttctcatcagcattaagcagagggaggcacgagcaggagccacctgcacactcaatgaggagctgaacagggatcaattacctttttttttagttattaggatgctgctagctgagaatctgccttgccttgattaccccaatgtctggtgcccaagtcccttgagtcctccagcaggaactcctgtggcatcactcaggagtctagtctaagaagctagctctgaccagggcagtggtggccaggcttctgtgagtgggccagcctcccccgggtaggacacaagccataccagcagggctgtatgtgaactgtggaaaatagagagcaaagtgggtaggtgggtgtagggtgctgttttcctggaaatatctacctaatctcgctcttctcttacctctag

SEQ ID NO: 55 - RNA, 2866 nt, Homo sapiens
  source 1..2866: mol_type = other RNA
gtacccctgctgcttatgcagtccacagcttgaggcagttccttggctcagagcccagctggttcactgggcttgagttgctccaaggctcagatatgcctcctacagagagccccacccacaccacggtccctaccaagtccccaccacatcctcatcacatccttgctaagtccctgccactgtgtgttctgtgctgaagaacttttcattcagtagttgtaggggttcctattgtaatcaggaaaccatctggatagcatgggagagcatttttgaaaagaactttcccatgtttttgcttacagcaaaaaagcttggatttggggaataaggagcagagaaggtaatagagaatattagaatgttttgggtgcttgacatctatgtctggacatgtgtttgagtttcaagggaagggacttaactggcacatcatttcagtgtcagacacatttggttagatcaaggaatagcatctgttgtaggaagagggctctttgttctttataaaaattacaagaagatggagaaagaagcaataggaggtatgtctcctggcttgtgataactcttggaataggtgcttgtaggttcctgccctggcacagtgccccatgtaaggagcacaccacccaagaaggagagagctagagcaagtactggaggaggcaccagcatcccaatgccttggcttaagcctgggattgtagagggatgaattagccactctcttctgacttacctggagagtaaatcaaatcaaatcaagaagcaaggatatgcaaaaaccttatttccccataaagtttttattctgcccagtttctggattgcaagaaaaaccaaatacagctaatgattgaaacactgctgtctaaagcagtgcttgtgatgaattttttcccttcctcttgaccagcagagacctaatggctacttggcaaaactgactttgtcttcccaccccttacctgccagagggcccagaaatgcctaaggctcctttagttacagaaagtttgcttttactgagatcttccagccactgattcccatttatagatctggtgattgctgttgacatcagttgaaaattatttttaaaaaccacttgcagttgcaaatcctttttataactctgtaactcagaatatagaattgggtagcaaaattgtttcccagaattaccaatggtctccccacccctgcctggcatgttccctcttaaaggactaatcccaccacatcacctctgggccaggcagaacatcaggggtgctgatgttctgtgatctacagcagttaattccaaacttttctcccttattggatgagatcatttttctattgtgttttttacatttttgttcacaaagattagaaaacctgcaacacacttattggcatatttttctgataattttcatccaaaacctaattctgactttacaacatactatctttacaaaggtttgcaaaaattctttcatatagcattgtatatgtctgtcatgaaataatagtaagtatattattgtttacattataccacttcaaaataatttcctttaaagtattcttcaaacaagaaaaaggcaatttctctcaagaagttttagagagaatttacaacttgctcctaagcaaatgtgagaacttcaggaggttcatctggccattggctttacaactccaaattgtgagccaggaccacacagatatttctctagaaatcagcgtttgcttaccaagaacatttttactctccaaaggactccatcctggaaaacatgttttgggataaggtcttatgcaatcttatactctgttattaaaaccagtgagggtcaaggtgttaatagattaagtagtgacagatgatcagacaacttagaaacatcctaaataggttaataattatgtgaccatcgcatgtgcattcccaaattaggaacaactcagatcaatttctaatccttattcttacactgttccagttcccccatataactcgtatctttgtgttagtttcagaagtttctgaagtaccctcagccttgatggggatcctcgcaccacctcaaatcctgttctcagccctaagaactgtgttagtcatcctcttaagaggatgtgtgattttaaatcagataatgggataaaccacatttcgtctagactggtcaggcctttgtccagtcccctcctcgcccacactaccccagctccacagcgggcattggttcaggaattcaacccacactttataactggagacagtatctctccagttaaaaaggtcaccttggtgtccgcttctcaaggaacatggacatctttattaatcaaagcccaagctttgatctggagcctaatatcctgcactccagctctcatctctcccctcccccagtcacactttcatgcttcccagagccacccctacaggaagtggtcaagggaattctatacctcagggctgacctaaattaggatttcttggcttttaagataatggtaactttcttaagctaaaaaagccccaaaagaccctgtaagagcccttggaaacagcaccatgggtgtagcttccccccaggatgtaagcatgtatgcacacatctcgtatgtgtgtctttgtaacaaatgcctggatcttagtaccagggagacctgattcatagatttcatagagaaggagagaaagatggcccataacctgggtgatctgacagaatcacagtgccctcagctgagtgcccttcagaaattgattgacaactgtttagcttttgaaatctaaaagtagtacagcatctcagaaaaccaagatgacgcgagtccatgtgatctccttccacag

SEQ ID NO: 56 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 57 - DNA, 4393 bp, synthetic construct
  source 1..4393: mol_type = other DNA
cagagagggagtggccaactccatcactaggggtagcgcgaagcgcctcccacgctgccgcgtcagcgctgacgtaaattacgtcataggggagtggtcctgtattagctgtcacgtgagtgcttttgcggcattttgcgacaccacgtggccatttgaggtatatatggccgagtgagcgagcaggatctccattttgaccgcgaaatttgaacgagcagcagccatgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccaatggcgccgcgtgagtaaggccccggaggccctcttctttgttcagttcgagaagggcgagagctactttcacctgcacgttctggtcgagaccacgggggtcaagtccatggtgctaggccgcttcctgagtcagattcgggaaaagcttggtccagaccatctacccgcggggtcgagccccaccttgcccaactggttcgcggtgaccaaagacgcggtaatggcgccggcgggggggaacaaggtggtggacgagtgctacatccccaactacctcctgcccaagactcagcccgagctgcagtgggcgtggactaacatggaggagtatataagcgcgtgcttgaacctggccgagcgcaaacggctcgtggcgcagcacctgacccacgtcagccagacgcaggagcagaacaaggagaatctgaaccccaattctgacgcgcccgtgatcaggtcaaaaacctccgcgcgctatatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattacccagaaccgcatctaccgcatcctcgctctcaacggctacgaccctgcctacgccggctccgtctttctcggctgggctcagaaaaagttcgggaaacgcaacaccatctggctgtttggacccgccaccaccggcaagaccaacattgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagcctctccaggaccggatgtttaagttcgaactcacccgccgtctggagcacgactttggcaaggtgacaaagcaggaagtcaaagagttcttccgctgggccagtgatcacgtgaccgaggtggcgcatgagttttacgtcagaaagggcggagccagcaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacgtgcgagagaatgaatcagaatttcaacatttgcttcacacacggggtcagagactgctcagagtgtttccccggcgtgtcagaatctcaaccggtcgtcagaaagaggacgtatcggaaactctgtgcgattcatcatctgctggggcgggctcccgagattgcttgctcggcctgcgatctggtcaacgtggacctggatgactgtgtttctgagcaataaatgacttaaaccaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcg

SEQ ID NO: 58 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLPERTAMSLPGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 59 - DNA, 2214 bp, synthetic construct
  source 1..2214: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcctgagcggacggcgatgagtcttccgggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 60 - RNA, 565 nt, Homo sapiens
  source 1..565: mol_type = other RNA
gtgggtggatccttgtaagcaggattagcgagtcactccacgctcaggttctttagcctgagggcccgtgtgccacagcatagctaccccgcccttccagcctcgggtccctaatactgccttgcttcggttccagtttccgccgcacaacttcactcattccaaatgttaatttctgcgttttttttcagccccaattctgtttctccaaatcagggatgattgtcggccttccacagaccctcgcgcttgccaggattagggtgttcgcgcgcattgtgggtaggggtgtggaggaagggatccagaaatcttaagtattaacttagattagtgttagcaaggaagccgtcacattttatttagccgggacactctgacagtttgtgccgactgctatttttgatcaaggctattttgcccacttgtctattttgtggcccaattgtctgttttgctaacatcagaaagttataatgaaataatctgcaaaaaatgtaaggtgctagaaaaccaataatactgtgtaccttgaaaatgctaatatacacctgttttgttacag

SEQ ID NO: 61 - RNA, 172 nt, Homo sapiens
  source 1..172: mol_type = other RNA
gtgcttaattggtcaataataatagatatatacattaacttatgattaatttattaataaaatatgaatttatttttttcagggacaactataattgtcacaatctggaagtgttcttatattttgcttgaaggttataaaatataaaacagttgcttttctgtttacttag

SEQ ID NO: 62 - RNA, 1391 nt, Homo sapiens
  source 1..1391: mol_type = other RNA
gtttgtatgtagtaggttttaactataggtttggctattagtggaactataaaaatctgttcttatataaggtaatctttgtgaaaatacctggtaatatctacatcaccactaaaaaatgcaatatatttaaatgtgaattaagtattttagtgtataaaacattgctagtttctacttaaagtttctaaaagggtgtgtaggggaaatagaatgagtatgttgaaaagtaacataaggaaatatatcttgaggtccaaatgacaaatgcagacaatgactgctatagggatttgttaagaggggaaatgatttaagagatgtcagaagacttcacaaaggatcaatactgaggagtagtgttagataagtggaaggcaatgcagtggtaagatagtaagggaattctagagctgttggttaccataaataaatactgagaacaggaaatatgtttattctttatatttgaggaaacaaggtgcagcaagtttgtagcagactgtagagaaaacaaatcttgggtaagtactttgagataggttgttgagggccttaaaggtgtattttatgctatcagcaattgagaaggcagtaaaggttttcgaaacacaattgataggtacaaaaatacaccttaagaaggcaaaactgagtatattatgtaggacaaactgaaggaaattggagctttgtagacatcacattatagcggagtttaaacctgaaattatggattagaataatagcaattggaacagaaaaaaagtagtggaaagacattacaaagggagatgttgcattactggatataagacttgaggacttgaggtaaaaaggagaatcaaaaatgtttcatgctattaaaaatctagaaattgtagtcttaagtaagaaaattgcctggcatggtggctcacgtctgtaatcccagcactttgggaggccaaggcaggaggattgcttgagcctgggagttcaagactagcctggataatatagtgagtccttgcctgtacgaaaaaatttgccgagcatgatggcacaccaagcatgatggcacgccaagcatgatggcatgcacctgtagtcccagctactcaggagactgagatgggaagattgcttgagcccaggaggcaggaggttgcagtgagctgagattgtgccactgcactccagcctgggtgacaaagtgaggccctatctcaaaagcaaaaaaaacaaaaacaaaaaccaaaaactatttattcagcaaatatttactgaacgtctccatgtgccagccattgctggcactaaggatcataacaaataaaacagaatttttattttcagtgcttacattccagtataaaggcatattgaaataacctttttttaatgtttag

SEQ ID NO: 63 - RNA, 303 nt, Homo sapiens
  source 1..303: mol_type = other RNA
gtaagcaccttggaaaaagtttattatggtattaaataatgaattccatttgttcattaaactgtagaaaattaaattatattctataaaatatatatattcagtttatttttaatatataacatttaataataaatatttctagactcctattttatggatctgccatataatactttttgttaccttataatcatgatggactcttttaaaagaattaattttgttattgaaatttatttaaaagtttgttttgtggtaactaatcaattaaaacgtttttctttttttttaaaaaaatag

SEQ ID NO: 64 - RNA, 2358 nt, Homo sapiens
  source 1..2358: mol_type = other RNA
gtatgtctttttgtattccctaggatgtaattgtcattaattttattttgaattgttttcaaattttaaaattattgttggctggaaaaattataaggatgattgtaatcatggttatttgtttattctgtatatgttctacatgcctattatgtgccttatatagtactaaggactgagcatatggttgtgaacaaaataagaagttaactgctggatggagcttatagtcttgggaaatatacagaaagattactagtaactgaggtggagggtgggtggggatttgaggaatagtgacgaaagggtgttatagaagtaatttttgacaaagctgaaggctaaaatatgaatgtattgttgaagaacaaaatacattgagattcctgagaaggtaggaatgtgatacaaatggatcagcctttgaaaggaggaatacccttttcctttgtgttaggagaggaggatgagtggatgagcgtgggaagagtggatgtgtatagaggcttttatgtttgtaggcataatgcttggaagttgaggggttggtgatgacatcttctgttaaaaagagtgggaaatggtgtggtcacattttaaggaaattaggtaaaatttgaaatatattggagacaggactggagagttggggatctggagtcagacagatttgagttctagtcctgattcttctactcgttaactctctgaacttggatgacctattgtttttgattgtatatccagctcctgggaaaatgccaagcactttcaataaatactaaatgaattatggagttggatcagttctgtgttagtgtttagctaggtagctgctgtagaatagaagggtagcacagttgaagatattggtaggaaagtggttgaagtgatgattatgaagtcttaactgaatagataaaatcaagattggggttgggtgggcagaagggtagggatatggagggagaagatgaggggttagagtgtcctgtgaggtcgaaggacaggcatagtgggaataattgaaagaatgttctggttggacaaggatctgatgtgggtgtgggagtgagagactatagtgaattcaagaaaaaaatagactagaacaaaagttatgtggagattgcttagtgggcatttgatagacatctgtgggccacatgcttaaattcccagtgcattttgcggagttactggaaggttggtggcttgtttctaccatgagtaggtaaagatggagagcaggatattttgtgagaaagcagctgaagtttctataggatgatggaggaatgataggaatgatcacctgaagttgcagggtggggtaaacctagaagcaccaacaccttcttctgaccctcatgtatttggaatctgaaagaatgagcaccttccaattgaaagagttccaagggcattagtatactaaaggatccaaattgcagctaagccaaggagatggaaaggaggattcagtaaagaatctgaggatgtgaaatattaatttatcttggaagagaattttagagagcacaatggaatgctttttggaggagagaaagagtaagaacaatttggttaaggtagaggaataacagaactataaggtgaagaaatgaatgtgagacacattagatgaccaaatgatttgatgttcttggccatgacctgaattaacaagactgtgaggtaaaatggatttaatcggctacaaatcttaagataaccaaaacctgagctgtttaatatggtagcactagcactaaccacttgtagctatttatatttacattggttaaaattaaaatgaaaaatttagttcttcagttgcactagccacacttcaaatgcccgaacatagctacatgtagcgagtggctattgaactggacagcactgacagcatgtccattatgctagaaagtcctatgggacagcactggtctaaacagtgcatggtatgagagaaagggcaggttaaggcactcagcttcactgactggggtggagattctgatggtttgtactcaggttccagatccctgaggctcaggaacctttgcagtttagtctggttacctgtggcccagtggttacaacagaatgattaacagtcaattctttgcatctctgggtggctcaggaaaaatttaaggagttattagctgtgaactaaccttaagtaagttaaattaaaaaaaaaaaagttcttaagctaatatgattttaaatatctgcactgaagtataatgcaaatttaaattcagcataattatttgcttgttgttgactcatttgaacctcaaaatataatgggattaatttatactttgggtttattactttaag

SEQ ID NO: 65 - RNA, 5424 nt, Homo sapiens
  source 1..5424: mol_type = other RNA
gtaaagcactttttttttccatgaatcttcactgttcaagttacctggctttttattattattggtaacaatatcaatttttatattgtatgttatatttgaaaaatgatgtacacttatctctaaggttttatatcactgttcattttgtcatcaccaattttaaaatataatggtacttctagtgaatatgacttgaagattaattctttatatttggaagtacatttttctcaggacatcaaacttgttacctaaaattaatgcttttgtctggaagattggtatcaagtaactaatagattttcataaagaagtgatctttctagtgccatagtttattttgggtaaaagttatatttgttcatttcaatgtatttatatgattagtagattcgcaaatgaatctttcgatatattcaataatggttaattaaatatcttgtttttggttgtaccttattttatgtgagatatatatatatatgtatagtttttgaaaagttgtgttcatgtcagcagtttataaatcacatatttaaaataacatttttaatgcatagtttttattacctcgttattccttgttataaactaataattcttgcagtgttcacttgaatttagttttaggaaaaaagttttttgcagatcaacttgtatttcctggaagaaaatttcctattttacctcagcttcctatttaatgtattatttatttatttacttaacatttatttgttttttatttcacctgaactgttagtaaacttagtaaaatttggtgcctacatgtggtaactgtcctgtcccttatactcagaaacgttttccacctttgtgtcctttaggtcattgttgtgttatattccatttattttattttgtccattgttctctcagaaattgagggtcatacattttaagaaaacaatgatatgctatttaagagaatgtatcataaattgatttgtaaggaaaagtatccccattcttcatgtatgtattttactctaaaatgttgaagaatcatatagaagttagctatgaaaacaatgtggtagagaaagtatggatcgatgccacttaaatgttaggaagaagctcttagagcattatctgtttagctaactgcaaaacatagcagacatgtggattttttaatagtcatcaaggatctaacttataatatacactggtagaattgcttagggggatgtctgtggttttctggacttttgttcttctatatagacctgtatcagttgacttatcattcataccacacacccttagctaatcagaactaccttgtccatttatatcttagactattgtcttttttcatagtcacacacagagaaaacttgaatatatggcctgtgttcctttttggctgctcaattccttgagatgaaatatgggtatgggttgctttggcaattacttctttgccgttaaccagtcattcagttttattgagtctttacagcataccagaggctgctagttactagtgatatagtgggcaactatgttctggttctcaagaatattcatagtcaataataagcataacatagtgataatatgatacttagggagatacataaggtcatattctggcatactctggagagagataccgtaatcagccttgaggtgcaggatgtgatctgtaaactgagacctgaagtatagttagactggtaagaggaatgaggatatatatggtggttaataaaagaacattctgggtagaagatatagcatttgctaagacctagaggtaagagatgttatggagtatttaggaaactacagttattcattttgactgaaatataagtgaaaatagctttcatagagtccttactatgtgccaggcacttcatatgcattaattcattattgcttatttgatacttgtcatatgagatagttgtcatttctgccatgatacagatgaagaaatggagacacagaaagagtaattgcccatggttgcacagcttataaatggtaaaggtaggatttgaaaacagtcttactcaagagtctgtgctatcttgccttcccagttttattttttatgatcctctggagagataagcaagggccagttcctaatgaatttggttcttttcctgaaaggagccagtgaagagttttgagcacaggatatcatgatcagatctatactttaaaagtttactgtactttgtagagagtggattgaaaagggccaagactagtaaggaaacatttgtgttaattcagggaagtgctaatgatggcatttgcctgagaaagacaagtgtgagagaagtagatgtaattggatgtggtgaatgtaattggttgttggaggagagggaggatggagagtctgcctaattttgtgggttgggccactaaataggtagatagtgccattcattaaggaggaacacaagaggaatttggaaagcttgagattatttcagttttgtagatgttgagtttgaggttcttctgggcatattcaaaaagggtatctgtggatatggaattcacaagagaccctgtacagatgatgaggatttatgaatcatcaatgtagacattattgaagccagagaagtgattgtaaggcacgtctctgagaaatgtctaataaagcaatgaaataggaagagtgcttcaaggaaaagctcaagaaaggagaaacagagtgtgatgtttgagaagacaagggaaaaaaacattaatagcattaaatgctttagcattaagttcttggcttctcttcttgtaaaaatttcccaattcagaacacagtgggattattaactttcaattgataataataatgataggcaaacttctaaaatttgtattgtagtttgcattttattataaactttctttaaatttttattttgaaaaatgtcatatcttcataaagattgtaagaaacacactgttggtgttaatgtaaattagttcaaccattgtgggagacagtgtggcaattcctcgaagatctagaagcagaaataccacttgacccagcaatcccattactgggtatatacccaaaagaatataaatcattttcttataaagatacttgcacacatatgttcattgcagcactattcacaatagcaaagacatggaatcaacccaaatgctcatcaatgatagactggataatgaaaatgtggaacatatacatcatagaatactatgcagccatcaaaagagaatgagaggtcaagcgtggtgactcatgcctacagtcccagcactttgggaggccgaggcaggcagatcacttgaggtcaggagttcaagaccagcctggccagtatggtgaaaccccatctctacaaaaacaaaacaaaacaaacaaaaattaactggtcatggtactgtatgcctgcagtcccagctacttgggaggctgaggcaggagaatgacttgaacccagaaggcagaggttgcagtgagctgagatcgcaccactggactctagccttagcaacaaaactagagtttgtctcaaaaaaaaaaaaaaaaaaaaaccggaacaagatcatgtcctttgcagggacatgggatggaggtggaagccattatcctcagcaaactcacacaggaacagaaaaccaaacactgcatgttctcacttataagtgggagctgaacaatgagaacacatggacacatggtggggaacaacacacactgggacccgtcaaggggtcggggtgggagaacatcaggaagaatagctaatggatgctgggcttaatatctaggttatgggttgatctgtgcagcaagccaccattgtacacatttacctaagtaacaaacctgcacatcttacacatgtaccccagaacttaaaagttgatgggaaaaagaaaaacaataaccacccacatacccttcatatagattcaccagttcttaatgttgtgccaactttgctttatctttttgtcagtatttttacacacacatgtatttctctgtctcttgtttgttcaatcacattttttgctgagtcatttaagagctaattgcagatatgatactttgcacttaaatatttcagcttgtctgtttgaaaaagaaagatgttctcctacaatgaacacaatataattgtcatgctcaggaattttaatattgattcaacaccattatctagtccataatgagatttcttctaatggcccaataatatccttcagtctccccacctccaatatccaaagttctgtcaaggatcacatactacatttggttctttattatagactttttaaatatcgttgtataccattgtgattctatcgtctcctttaataaagaggagaaccagaaaaatgaaaggtcataagaggaatgaggtttggagaataggtgaaaaaaggcatcataatgtttataataatgtttgcctgttcagagaaacaagaatcacagataaagtcacttatatgtagataagagaatgctgtattactttttgctattctattcactgatcatttttctaagaactctgtatgcttcttgtttaactcttatgtcagcatgtatgagaaaactgagttaaagagatgttaagtaactcattcatgctttactagaaattggttgatgagggacataaacctaggccggtgtgattttagattgcttcttttaaccattgtgttgtattgccttatatttctaagtaatttatgttcactgagagcaaataatagtctagctatgacttagaaaagtaaaataaagatgttgggcagaaaaccattttattaggggtttttttggaggagcagattaatttgtttctgtattctttggttagtttgtgtgtgtgttctttttaattctttaaaatgaaactgtttaatccttaaatccttaagttttgaaaattttggcctattatttatgtgttaggttgatattaaatccttaatagctttaacattttctactttgttagagaggatttaaaatttaagtagataagctgaatatctggctttatattaaattactgctgatggccaggcacagtggctcatgtctgaaatcctagcactttgggaggttgaggcagatggatcacttgaggccaggagttcaagaccagcctggctaacacagtgaaaccccgtctctactaaaaatacaaaaattagccagttatggtaatgcatgccagtaattccagctactcggtaggctgaggtgggagaattgcttgaaccgggaggcagaggttgcagtgagccgagatcgcaccactgtactccagcctaggcgacaaagactttgtctcaaaaaaaaaaaaaattactgctgaattttatcttcttcttatttattttttttttttactattttag

SEQ ID NO: 66 - RNA, 599 nt, Homo sapiens
  source 1..599: mol_type = other RNA
gtaaaaaattttagtagttgtggtggttcaacaaaggtacttattaaaataagtacctaagtttacataaatttatattttaaccaggactggagtcttctaagtaactgatgttttcagactgattttatggtatgactttgtctcagggaaatagaaaacaaagcaaaatgtgaggccattaagtattacattcatctcaggtctatgcgggtaaatctttttttgttgttttataagccattctttgctagttttctaattgaatagatgactggatttctattcttatttctcttacccagaatcctttaaaattttttgttacttgtggaatcttataaattctgattatcatttggttctactgagccaaataatgtttgtacattgtttattctgatagaagttcttaagtttctaacataattgaaatattatttgttttggtagataattagtattctttctttggttattcaagataatatgcatcattttcccaaaatttttttgttttctttagtttctgattattatttttaattatgtattacctttctcatttctaattaccgttttcctgtccttttctgtag

SEQ ID NO: 67 - RNA, 124 nt, Homo sapiens
  source 1..124: mol_type = other RNA
gtgaggctttaagtgtggtgaaatcttgggaatttaaaatatgttgtgagagcactatttagaggatatgattttgttattctgaatagttttgtaattgaatgttgtgtttggttaccttcag

SEQ ID NO: 68 - RNA, 391 nt, Homo sapiens
  source 1..391: mol_type = other RNA
gtaaaatggctagaagtcaattcagagcaatggttcctaaaaactttaatttcattacaatgtaaatataatatttagccctacatgtaaattccctggtataaatctgtcactatgtacttgtaaaatgtgaaataaattacatctttgaagttgcaactttttagccatttttatatttgcctgtcttggtcattaagaacaattgaggtccttatgtactattttcttgattcaatttgatttaattggtcaatgccaattagtaaaggtctataaagaattctctttttttctagaggacacttatggctgcgtttaattttaatttggtttaaatttcagtttttttaaaattactttttaattatagtgtctttaacttttttag

SEQ ID NO: 69 - RNA, 658 nt, Homo sapiens
  source 1..658: mol_type = other RNA
gtaagaattacttttagaataacttatttattcagacttcatattatctcattactatttatttgacactagaaagtactttttctaggatgtgaatttttgtctgtctttttaatagtgtaatatcttgtcatgttggtatatttgtccatatgtgtttctccaatcacctcacaaacactaatttttgcaatttaggatatataaatgatacttgaatgaatgtgtagatagcagtcattatggggttttctataaaagactactgaaaatcctgtggatcataacatttcattttatcttaaaataaatacattataaatgtattagaaaccaatacattgttcagtatttatgtggattaaatttgtttaaaaggtagaataatgtttaaaaataaaattttctagtaatgaaagataattatgcaattataagatgcagaaactattaaatgtcacctataattccaggatgacttcaatgataaatacacatatgtaatgtaatgtatccgtatgtatgtgtatataagtatgaatacgtatgtgtgtgtatgtagatatatttatatatataatgtatatgtaaatatgcacaggtgtaaatatatgttacatcagtttgcaacaactcttgaaataactttgtcttttag

SEQ ID NO: 70 - RNA, 2507 nt, Homo sapiens
  source 1..2507: mol_type = other RNA
gtattttttttcaattgacataataactttttctttttgtattttagatttaaattttagtcttatttttctttaaatgtcttatactggtttataacacgtttattagggtttttaaacataagtttattttatttattggttagaaaagctctagaactgtcctttttgatctctagctaatttgttattgaatgacctctttcacatcaatgagtttaactttaaactttttgatagaagtctaactccaaaatatatttggcatctaaaatatataattcgaaatataatttaaatttttttacttaactcatagttaccttatatacattagttaaatagttgcaggtttaattttagtttttctaactaaatgtcaggttcatcagtgggaatgggaataagcaaagggatcagaataacttgggaagccttttcaaaatacacttttcttcctcaccaccactctccaaccttaaccaaattgtcaggccttaccatattagaagctgggattatgatggttgtatacttgaaaaacatcagagattattctgaatgaataattctaattttaaaaactatcacttctagagtcattgctttctagtatggttcacataaatcttgtgggcagtttggaactggttagcatctagggagctcagataacctatattttaaacaaaagcattagcaatggaaataaggcctatagaatcagtcatgtctccataaactttatataaagggccagacagtgaatattttagaccacctggtctctgctataactaaactctgcttatagcatgaaagcagccattgacaatacgtaaatgagtgagcaaggtggttttccggtaaaattttatttacaaaagcagatgggaggccagatttgacctttgggccatagtctaccaacccctggaaaaaacagttgtctttaccagattgaatgttggcagggtaaatggtgacatgttatatgtattctgtactttgttttgacttaataccatttcataattattttatatcagtacgtatagtattgctgttctttttaaaggctatgtaatttttctttttatacaggtgttaatttgataatttgtgaagtttatgaagtttccaattttggggttgtaaactgttttaatgaatatccttatatatgttattttgcaaatgtacaagtatatctgtggaataaattgctgcaagtgttgtaattgtcatgtatgttgcaaatacattctaacagtttgtcactttttttgctttatggcattttttgctgtgaaatatttctttttatgcttagttaaatttattattttttaatgacttttgacatttgttataatgagaaaggcttctgagtataaacttgttttctcatcttttctcctaatatcttgttttgtttttgtttttgtttttgtttttgagacagagtctcactcagttgcttaggctggagtgcaatggtacaatctcagctcactgcaaatgccacctcctgggttcaggtggttcttgtgcctcagcctcctgagtagctgggattacaggcatgtgccgccatgcgcagctaatttttgtagttttagtagacatggggtcacactgtgttggccaggctggtcttgaacccctggcctcaagtgatcctcctgcctgggcctcccaaagtgctggaattacaggtgtgactctgcctggcctttttttacatttaaatcttcgaaacatataattcattttgatgtaaggagtatcatgtggattcaacagagctactctgttgtccaaacatcttttattgattatttcatcttttattgaattgattgatctattttctagcagtgtatacttgttttaatttgtgtatgttttaatatctaaaaacgttattatttttctgcttttagacttctttatgaatatttttaatgtgaattatagaactggcttgtccagttcttaaaaaatatcttgtggatttttattgggtatgtgttaaagttataaattgttttatagattgatttaggataaacctttttatgttatttggtccttctagctaaagaacacaagataccttttctttcattcattcaagatattttatgcctcttggttgcattttaatgcatacttcataaagatcaattgtataaaacttttcacagttgtatggaagtacttcttgtttataaatgagttttgaaaggttgaaatatttttaaagattgaattataaaaaaagaaaattcggtatatattttaaaatcattttctatttgaatttcaggttgtatatacaaaaggaacagagattatgccagtagttgctcatactttctcatttcaaataatttttattttctgtatcataaatctactaacggtgtttattatttatgataatgaagaatgttttattaactttccttttgcataacagattctattgtgtttatttctag

SEQ ID NO: 71 - RNA, 946 nt, Homo sapiens
  source 1..946: mol_type = other RNA
gtaaaatcttaacagaattttgtttatcaaccagttttattacagttggaactctgaacgatgtcttttatttattatatcatcagtgcctagtgtagcggctggtactaccaagtgtataataatgtcttttgaaatttcttctaccacctggtcccaataaaaaattagaattaagtttagatcacggattagacttagaactagagttactgtgtttatttttctatgtttatgtggatagtacacacattgttttggttagaaattatttaacaagaaatgattaaaaacttttagaaatttaaaataattttatactcttttaaggtttattttactgtatcttagtcctaacataccctatacaatgtgaaataagctaaaagcatggttataatttgactgtgctacctattttatttttagtgaaaataacccaaataaaaggaagtaatacttttattatttgtgctgtagttatagtccacaagtaagaagatgatttgaaaagtgtatgctgaataagaacaattacaggggacaacattttttaataaagtacgaaaggggaaaaagctaagttgaataaaagagaaagcacagagcaaaacagaaacatacaaaatggtaaaaaggtggaattgaatggaggatgaggaaagtaacatataaggaagtatagaagccataaacattagggagttctggaaatcctattttccagagtgttagccattatatccatctttcagtattggagtaacagcagtgtacctatcattgtgtattacagttgaagtgtacaaaatggtaaaaggcatacttgtacccacaagaaaatatgttctacagtcttgttgaaaaaaatcagacgtacttttttccttacctttttaggttaatattcatgaagggatatatattgttttaaaatattttatag

SEQ ID NO: 72 - RNA, 4079 nt, Homo sapiens
  source 1..4079: mol_type = other RNA
gtatttttataaatatatagttattttatatacaattatgtttttaacgactttatttttattaaaataaaatgtcaagtcaatattgagttttctccatttgaattttatattttcaaaaaattgtacaagatatttattattatacttatattactagtgcttacatttgtaaatgatggatgcattttctattatttttctcctctggtgaaaattacattaacgtttattaccaggtcactggtatgaaagaaatgaaaaattgtgatacaattatttttatttaactttttataattaacaaagaatggaagataataaaattttgaccagtgtaacagcattgcagatagttttcagaggtaatttcacattaatcttacccaaattaatgtttcatcatattctccttaccctgagccatattaccttttttaacacatcaaattctatgaatataagttcttacaatatctgtgttgttatatttccatagcactacatactatagttatgccagggcacactagtgcgaactgttcatgggaaattcatggacatgtttattataattggtgactatgtatatatgtatacactacatttatacacacgcgcatggaatcactatttcttcttcatgtcatatatatatacatatatacacatatatatacatgtcatatgtgtgtgtgtatatatatatatttgtatatatgacatgaagaagaaatagtgattccgtgcacatatgtgtgtgtaagtgtagtgatgtgtttgcaggtacggttgtaatttcaaaaatgaagcaaaagccttgctcaggagataattgaaccaatacttaaaggaagtaaaggagtgaaacatgcagatggctctaagcagtgggaataagttcaaaggcagtaaagcaggagtgtaccaatcatgtctgagaacaacaaagaagtctttttggctggagtagagtcagcaagtgaggcagtgataagaccagagaggtaaacagaggccatatcatatggggccttatagttcattgtgcagacttggcttttaagtgagaagggacaccggggaaagtttctgaagatagaaatgatataatttgacttaggctgtgtttgcagtagactgtaggagtggtaaataagaatcagggagacctgttagaagactattgcaataatctggagaaaagtgatggtggtttggggcatggtggtagcagtggagttactggatgcagcagttctggatgtattttgaaagtgataaaaatggaatttgctaacagatcagatgtaggatgtgagagagagagaactcttggtctgaaccaaaagttttggtcatggtggggttgtgggaagagcaggttgagagataatcaggtacttaattttagacatgttaggtttgagatgcttattagacattcaagtgaaggtgttaagtaggcacttgtatataaaagtttaaggtttaggacaacaatctaggctaaagatatgtttggtaactgtctctgtaaaagtaattgaaataatgaggctggctaagatcaccaagggagtaaatgtaggttaagaagaaaaatctaaagagcttctactttagcagctggggagataaaaaggagctaccaaaggagactgaaaaggaaagcccagagagctaggaggaaaagcaggagtatggagagccctgaaaaccacatgaggaatgtaaccaaggaagaagaaacaactgctttcagagctgtgttcattgctgctgataggtcaagatgatcactaaaagttgactattggacttagcaatggtcatttttggttcaagagaaaatgggtagagaggaaatgtaataaagaaatataggaacccttttccaggactgtttctataaagagaaggagaaaacaaggtggtagcttgaggggaaagagggattaagaaaacatttttctctttaagatggaagaaataactcatgattttaggttaataggagagctccattaaagaagaaacattaatgaatcaatgaagtggagagagagaacttctggaacaataatatttttaagaatgcaatgggatgggatcctagtgtgccagtgaagaggttggccttaactaggaacacagagttcatccataattgtagaaaagaaggtagagtgtatagatatcgatgtaggtggcttggtagacatcctggtaatgggaatttgtggaagttctaaactggttgctgcttttttctcagtgaacaagggagcaaggttcttagctgaaggtgaggataggagaagatgtttcataagtttgaggagaaagaagagaagtgaaagtataaaatggtcatctgaaagattgaagacgtggagaatgtggtatgactgttgagtaacttcaagagcccacgatatatatatgtatttctatttatgtgtttattatatttgtatcagaacactttgaaagtagtttaaactgctttaaaaggatgactaatagtatggattgtgcgtattctaattactaggagaaaaagtggcaattgatctctgctgtcaaataaggaaaaggacttatctgataacactttagtcagtccgtagttatataatccctaaagctcacagaaggtgtgtgtactagactgtactctacatcttgaacttaacttgtaaaacgtaatggctaatggtattcttccttcataagattaggattaggtttagttatcaggaacagagagctgaagaataatggcaaaatcaagatagacatttatttctcatctatgtaatggcctagaattaagcattccagggtgttgccttcatctgccccatccaaaatggatggaatgcagctttatctcatgtctgtgtcccaaacagcaagacagaggaagaggggcaagagttaaaagcatgtgctgaaggataggcaggtaaatatagtgtttattgtgtagggccatgtggaagaatgataggagaatagatatgtggatggaagggagaatagatactgggggacaactcagcctgtgtcatgttccacagcttagatgttagctccagacagctgtgctcatttcttaaaaacttttgtgatctcaaacgtactagttttatgcctaagtccaatattaaatatataacctatatattagtaaatgcttataatgaatgagtgtgagaatgatctgtcaatcaattttggaatgatagcaatattatgttttggtcttttaacaatttagtaagatattacaagtaggcatttaggaagtttttagcttagtttggattaaatttagctgcaagtgacagaaaaatcaagcataatacaataatttaaacaagatagaaatttatttctctataatatagacaaagttgaagcaactagggcaggatttgtgtgacagatgctcaaatatcccctatcaggaaccctgtctcttgttgctgtgcctatctcaacatgtggtttctaactcatgtgaagttgccaccctcatatccatgtggatttcagctagcaggaaggaggaaagagaagagagattactcctttattttaaaaacatttttttttttttttttgaaattcacatatgaactttgcgtttatattccattactgacatgaccacacatagctgcttgtgtgtaagtggaaatttagttctttatttcaaatggccacgtgtcaagctaaaaatccatagttttagtacagtggacaaaagggaggttaaatattaggaacagctagcagtctgtatcacaatgatcattttttgtaaagcagtattttgcaaccttttaaaatccataccccttcagctaagaaggttttactgaacttcagttttttagtaaattgtattagtaaaaccaaaacaaaactttcatcttacaaatataaaatgacaactttaaaggatttttttttaatggcataccacttttcttgccaccatgttgggatcactgatttgaaggaataagtagtcaattcaattcatgatttttgtttttactctgtag

SEQ ID NO: 73 - RNA, 720 nt, Homo sapiens
  source 1..720: mol_type = other RNA
gtatgtatttttatcttgtcattcaaggagcttagaattattcttgccattcacagactattctgtgctatttactgcataccatttaaaaaacattccataagtatcttttgataaagattatcctcattaatttatactaaactattgaaacctttgagcatttactttttgccagaattgttttcaaacttttgatcacagtgatttgtccaaataatcagttttggtgaagcagcaggattacttttttttattatctgtgttcattgggccaccatgtagatgtgacaccactggccaatttgacagaatttatgacaggaacatactgtgtcaatacaacctgctctccactttttatactttttcattggttacaactaattcaagcaactaatgacttacttattctactggtattgctgatttgcttttactaattcttttagtattttggtaagtgttttttatatgtaatgcatattcagagtcactttgcctttaggatattatactggaaagttttaactgttgcatattacatcattattattactggatttggtttataaaagcacaataaaaaaccagtgtaatgatataaattataggcatatgtacattttcctttagacttagtaaaaaaaaaatcatgaacttgataaatttattcaagtaaaccatgttatattttaaattaaattggatatttttcag

SEQ ID NO: 74 - RNA, 1370 nt, Homo sapiens
  source 1..1370: mol_type = other RNA
gtaagccatgttttaagttacatagtttgcgcaacctgatttacaagtctttttttttaatttaaattttgtttattattatttattaagtagtttaatgcttttttcaaatgcttttataaaacatttaatacaaataaaagtggagctaacctgattgaagtggaatcagattttatggggttggagtggtgggtgggcagggctggaacattgctttatttggtctagcatctcctcagtaatagctgcttgtttaaaaagatgaaagtttattaataccacatatcagagattaacctttttttttcccaacaaaagtagggtctgtattacccatgtttgtttgcaaaatgctcttgtaacagatgagatatttaaacttcttgctctgtgttgtgattctcctgcctctgcctcctgagtagctgggattacaggtgtgcaccactatgcccggctaatttttgtatttttggtagagatgggatttcaccatgttggctaggctggtctccaactcctgaccttaagtgatccacccgccttggcctcccaaagtgctgggataataggcatgagccaccgcgcctggcctgttaaaatcttttaaagatttttaagtacttgatttttataatttagactacttacgttttactttgttcgagtattttaaggagtaattagtaatatagcttgagagtttatatatttattttaataaatagcctattagttaatattactaatttgagtgttatgatagtgcagactaagttgctgctttaaaatgaaaataaatatctaaatatcaatttcattattgctaaatttcatttaatgctttcttagttaaaaatgatcatttgtaaaaactattatctaaagaaaagacaaatagacaaataagtattttatacagatatatatgtgtgaaaagtatctaacttggatccgtagttgtgctaggaccccaaattagacttctgatcaacttggactatcagatcacagccttctgatcaacttggactatcagatcacagccaagaatctggaagttcctaaagatgacttctggcccgtctaggtagctgtcatagacatcatattttctgtgcttaaaaagctccaaatcttggtttataatttcatttaggtttttgttaggatttccattaataattgtgataaaattttaacttgggttacagtttaaatatctggaaaattctttcacagaaagttacctcattcttcagtgatactggctaagtgaattataaccagttgcttgatggtatatgacatttttgcagcttatttgaatgtttttaagtttttaattatattgctttctattgtag

SEQ ID NO: 75 - RNA, 72 nt, Homo sapiens
  source 1..72: mol_type = other RNA
gcaagtgtggtagtcagttgattattttcttggctgaactatagagaaatactaataatttatactttgcag

SEQ ID NO: 76 - RNA, 1337 nt, Homo sapiens
  source 1..1337: mol_type = other RNA
gtatactcagttattctaaacctttaaaaagaattattgataagtgagttgtctggatatgaaattatttgtgtcttagctgtttttgctgttctattgtggatctgctacaaatttaataaatgacaataataacctgaaggagataagtgagtgtcagtgggttcagtcctgaatctgaaatagacaaaaacaaaacaaaacaaaataacaaaaaccaagcaaacaaaaaagaaaaaaaccttagaattatggaatttttgaaaagttttatagtatagtattttaatttctagacagcaccaatatgttgttattaataataataaaacttagtagtttttatgttaatatatgttactcaacattttccctttccttaaggactatgcattgaaaagcttttcttgtaagttattattattattattattattaatatttgagatggagtctgtcttgttctattgcccaggctggagtgcactggtgcgatcttgctcattgcaacctccgcctcccgggttctagtgattcttgtgcttcagcctcctgagtagttgagactacaggcgtgagccaccacgcctgacttatttttgtatttttagtagaaacagggtttcaccatgttggcccaggctggtcttgaactcctgacctcaagtgatccatccactttggctccccaaagtgctggaattataggcgtgagccaccatgcctggccttaaattattcttttctaagtgaaagtaatgttttattgaatataaattaacatctttcttgggtttattttacttgagctaaagagaacagttggttaagttttataatagccattgcagtgcttttttgtaagaagaccacacagaaggactgtctttttcacttgccccaaatccccaagcacgtatatgagtaatagcagagtggttctttttagcattatgatttctataatacatccaaaactttctcaagaaaaaacttcatgatttattagtacaataatcagtttactcattactcatcatttatatttactttatatgtcttttaactggtgcttattaagtagcactttaatatagaataggcaaagaatggtagagaagatgaaattcaaaaattaggttctcacattattaatagttcattaaaagtgagctaaatgagaagcttgtattggctatgtagaattttggagggattttggaaacaattattctacctttgcattaaaacttgattgtaggttttaagaattaaagtgttggaatagtaggagggttattttaatgtttttagtttgttaattctcttatatatag

SEQ ID NO: 77 - RNA, 1850 nt, Homo sapiens
  source 1..1850: mol_type = other RNA
gtaatagtaaagtattgcaaagagagtaaaggaaaatatttttttttttttttttttttgagacggagtctcgctctgtctcccaggctggagtgcagtggcgcgatctcggctcactgcaagctccgcctcccgggttcatgccattctcctgcctcagcctcccaagtagctgggactacaggcgcccgccaccacgcccggctaattttttgtatttttagtagagacggggtttcaccgttttagccgggatggtctcgatcttctgacctcgtgatccgcccgcctcggcctcccaaagtgctgggattacaggcgtgagccaccgcgcccggccaggaaaatatttttattgtgttttcatttcttccccctttatctcattcttgaacatctaatcttattattgttgttaaataagtagagggaaatatttgcttatttaacctgttgattcaaagattgattaatgagacattatttactctgaatacagattaggagttcagataaagcagagctgctgcataggagatcatcattcaataccccacagtcagatcagaatgagacagaagagaatatgaccataggatcattatcaagaatgttatctgaaattcaccatagtgtagaaagtggaatgcatccttttgtccctttaactagactttcttcatccatgcaagttaaagagaattcaactccagaaactattacaataagagagatttttaaagcaccatgtctgcagtcttcaagaaatctagaatcgttagtcagcacctttagtagggaaagccatgaagaaataaatgacatatgccttttttctgatgactgtatgaagaaggtgtcaagaagccatcaagcactagagaagactagttttgtacaaaaaagcaattcatcttttcatggcttatcaacagcttcagacataatgcagaagttatcacttaggcaaaaatctgcaatattttgtcaacaaattcatgaaaatagagctgacatggataaatcacaagtagcaacattagaagaagaacaggttcattcccaagtaaagtatgctgatatcaatttgaaagaagatataataaaaagtgaagtacccttacagacagagatattgaaaaataagcttaaggttaatcttccagaccctgtgtctattactgcacaatcaaaattatctcagataaattctcttgaaaatcttatagaacagttacggagagagctagtatttcttagatctcaggtgagtttttctccaaattatatttctgtggttgttcttttatgacgtctctaacaaagttctgtaacaattatagttagaatatttttgtttgcactttaacatcagttatacacattgtactttttaaaatctaaaatgcagtacattgatatgaactcattgacttgtctaatttattaaatttttctttagaatgaaatcatagcacaggaattcttgatcaaagaagcagagtgtagaaatgcagatatagagcttgaacatcacagaagccaggcagaacaggtagtgtaaaggcagaacattaaaagagatgattgtggtactaaagacaaaaaccgttatatctttttgcctcttaccatggatgttgggagagggagaaagtgggattaagatcaccatctgctttactgtttagattttagtttatttttatgattgctgctatgtcttcatagctcgttttttttgttttgttttgttatacttaattgatcaaacttttcttaacttgaaaattatagacttgtgatattttgttgaaaaaaatcaattttattctctctgcttttttcag

SEQ ID NO: 78 - RNA, 535 nt, Homo sapiens
  source 1..535: mol_type = other RNA
gtaagttacaattatcttttacttttctgttcttatttttcctatacttaaaatcatgggcctaaaagggcgttaacacattctctgttttctaatctgctttactcctaattacctctgtactgtatatacttcagtctgtcactatccagttgatttgccttgctgttttcattgtgagagaatgttactaatatgaattttttgtgagaatatataactcctttttcttgtgtgttcttcaatcaaaatgaagttagaacaccaaatttaaaatactttaatataaagcatagtttaagttaaggcagaagtatgccttatatacgtgtgtatatgcacgtgatataaataggtctgtcatttaactcaactattcacgttggatttatagttgaatttttttgtatgtttatttacatttggatttttccaatgatgtctttggtatatgtgaaatatttgtcatctgtatagcatagtgtaaattgtgaaaaagatctgatcatccaatgagaaaactgtgtaattacag

SEQ ID NO: 79 - RNA, 2561 nt, Homo sapiens
  source 1..2561: mol_type = other RNA
gtaagttatttttttcatgttaatgtttttcccctatcactttagagagattttctgctgtgtacagatctccatagtttctgatgagatatttttagtcatttgaatcattgtttccctgtatgtaaagtgtagtttttcttgagctgctttcaatacttttcttctaccaattggataattgttattaatctgtcttcaagttcactgacattttcctctttatctgtgttcttttggttcaagggtcagcttgagaccttgaggagttttttacaccgactttggagctcgtttttgctgactcttttcttattgggattttcctttcacttatcccatggctttgggctgtatcctgtggttttctagatgagaaagatgatagatctctgcaattgcaccctgccctatgactaaatctttaaaaatggcaaagtcaatctttgctggtcctgtcttccgtatttgaggggttttttcccaaaatctgcttgcttttgttcattttctagaacatctaggtagttttttttcattcattttttatttatgggagtgtagatctcttaggaacttatgccatcagaagtattatgaaatggctttattctaaatgtttaaagatttactcattgctacaagaaagatttagccatcactaatattctatatatatttaccatatagggacttgagaatttcacaggattcagtatctgtatataaacttgaataatatacacattttagattgttaatatttaagtatatgtcatttatgttatctgaacatatttagcgtacattgtcatattatttcccaaatttgtgcttgatttcaaatgggaaaaaaattcttattatttattgaattgtttttttaaaaaaatcatgattaatcagtaattggatactttttaaaataacactataattgttaacagagaatgagagtgatactggtatgttaaaaacttcctgaggcaagaaaataatttgattcccattatatctttctcatactgactttccttctctgattggtgattttgttttgcctctgccactttgaatgtctaaaatgattctttatgctttttttatgtgaacatcttttgtccgtgatgatgcccactactgatactgtgtcccagatcaaacttaattttccaagggcagctctacttagtgaccaaatgaaaacacagtgaatagcccaagaaatcctaacttctatttatgttgacaatctctggaccttcctgaagccactgtttgcatagacttcatttacttttatccgggattgtcattgttttttcagattcataggccctatctgaaattcacaaatcacctagcaatacttctctaagaaatcttcagaatccatgacaatttagaccagacaatgctggattatgcacttcagttcactttttgttactacaaggtatttttcagtgcccccaacagctatcttaactcattctcattttaccaaagtccatgtagacacggcactattcctcaatgagacaactaactagaccaccttgttgtcagtcagagtaccttcctctacctacttttatcttccttatatcctctttgagttagtataagttattactctgcatgacctgctctaatctccttcaggggaaggcttttacaaatctactacctagagttaaaccccagatcaccttcctgagtaggagattgcatttggttctattcattttaccttatttggcttctaccttcactttttaagacttactttgcctttaacagttttttccatacagttcatctaaagtccaaatatatttattagatgtgtgcattgtgtgtatatacttagatatgccactgttggagatttcgggccagtgatgccactctgataatattttaatatttgacatattatttttgcttactcattattcttagataatatcatgttatgataccttgcctttatttttatttatgcttcaactatgtggagaggaagcactgaaaaattcacttaattgaatgttgtattgatcaattgttcaatattgtattccattcctttgcgcatgctttgaatgcaggtgctatataatttcagagaaaaatacctcattttgactgtacaaaaaccccatgtagggagcagagctcacattgttttccccttttagagacaagaaaactaagatacagagaatttaagtcacttgcccagctgttaagtgactgattaaaatttgaaccctggtcatcttattcccgtctggttgtttttctagtctaccagtctattaagattagctaggtgttttttaattgttttaatgaagtaattactatgcttggtaatgtaaatgaaagttttatagattcataaataagaatttgaattggcatactttattatcatgcttggcaatgaaaataggaaaatgcttaaatgtccattttatttaaagacagactgttttttactatgattttactgtttttctccacatttctaatatataatataaatttgctag

SEQ ID NO: 80 - RNA, 342 nt, Homo sapiens
  source 1..342: mol_type = other RNA
gtgagaattttattaaataaaagaaaatgctaaacataagaatgtagatttaataggaaatttttaattttttaaaaagaatgctttatgagaaaatgccccttgaattaattctttcaatattaagaaactggatttctcttataaaattataagtggaaaataagtgccttataagattgaaaagaatacaaaaattctaaatctcatacctaggcatttctaagcagaaactgaagtatggttgaggtaaaattcctggcagggcattcacatatctgtcaatttgtctttctttgggtgtaagagttgtgattctcattgctggatttttttttccag

SEQ ID NO: 81 - RNA, 2020 nt, Homo sapiens
  source 1..2020: mol_type = other RNA
gtattgaaaattttgttacaggtattgaaaattttacatgtgaataacaaaaatcattggtagtatgtttctttatgtttttatttttattttactttattttaatttttccatcaccaaagcatgcagatagtacttttctcaatatttagtcttcatgtattcctgagttctcaaaatagtaacagtgaaatatattttttatggattttgatgttagatggattataaataaaagcaatttataccattcattccattcatctgcatgagcagcatgttcatacatcttgttcgcacacctgtcattcatgtgaaatatatggttcacaagcagaacaacaagcagctattataaagcagtgttaagtaaatgagcacttttatttcttgctgggtggaaaacaaaagaataaagtctgtcaaggctttttagtgtcatgatagaattgttcccctttttgcattcacaagtaaaaactactttttttttgagacagagcctcactctgtcactcaggctggagtgcagttgcgctatcttggctcactgcaacttccacctcctgagtttaagtgattctcatgcctcagcctcctgagtagctgggactacaggcatgcatccctggctaatttttgtatttttttttagtagagatggtgtgtcgtcatattggccaggctggtctcaaactcctggtctcaagtgattcgcctgccttggcctcccaaggtgctagggttacagacgtgagccactgcacacagccataagcaaaaacttctaaaccaaattattcttcatctttgtcttccctttacgcaataaaatgttaatctaccaccaaagaggaaagggtactctactatactacctgccctgggtttctcagttttgctgtctatataatggtcgttatgaatgtcctaatgacagatccttttcattattttatttgaaatttgactatctataacatcacatacattataaatataattacaaatatatgttcagaatcaatgaaaatatatttttgattatatgggccactatttctctctgctaggtgatccatttgtgagtatacttgagttataattattaagtactcatttttattttggaaattacagtaattcatctttttctcaatattgggatttttattattattttatgttgtctaaggacagccttaactacttattagaatattgctttgtatgtgatattattatttttaaatgtataattttaacattattatttctcttatttacctgaggtataggaacactatcagcaaatattggtagtatggcattgtcgtattttttgagataaaattcatgatttttaatctttgtataagaaatatatcagaagtttgtagtagattagagagtaccaactgggagtctgaaaagctgtccaaagtggcaaaacaggtacttagactctcaatcctaaggctgtatagagctataaacgtggcaagacctttggagtcagacagacccaaactcaaatgttggatccatgtatatggaaagcacctgacaacaagcctagcatatgtacttggtaaaaatgattgccaagtgtagtgttaatgagtttttggatattgagtaagttatttaaatttcaatttcatctttaaaatgaaataattggaaaggataatttgagtgagggtatgaaattatgtgttcataagagagggtatgtggccgagtgactagaggcgagtttataactattctatctaataaaactttgtaatctggtaatttgtgtgctaaaaataactttacctgttgtatagtactctttttttatgccttaaactaaagtgttcaaaatatcatggaaaaatgatctgtgttgcttacagatttggtgacttttaactttcctataatgttgtcagaatatgaatttatactttcaaattcagcatttattctattgtgtttttttttgcattcttatttctaaaccacttttcag

SEQ ID NO: 82 - RNA, 1967 nt, Homo sapiens
  source 1..1967: mol_type = other RNA
gtataggtattagcaaaactataaatataattgcagtatattcttgttaattgtgaaagtaacgtaagaataatttatgttttgttcttcccttcttcttcttcctttgcaattgtatttttttttactctggtaactactgttaggaacttatttatggagacagtgtagcttaatgattacattaagcctgggattatcctgcctgggtttgagtcatttaacgtttgctttttgtaagagcttgagcaagtcatcttacctatctgtgtctcagtttccttatctgtaagttactttgtaagtaatacccttttcataggattattgtaaaacgtaaatgaattattagatgaaaatgctcggactagtgtgtggcacatatgaacagtttgtaaatgttagctgttgttagcatcattcatcatcatcacaatcatcattgttcatatatgtttatagggaactaacatatttctccttatttctgtcatctcatctaaatcaatagaatgatttccttaataggaattagaatacctaatcaaaggtgatttaaacactaagaataattattatctgacctaaccagaaccacaaagctagttgtagggcaggtcatatttgaaggttgttgttatcgcctatgatggttgtaaaatagctgcatgaattcaagaaagatgatgtgcccattgaagaagaggagcatttttttctacatagcttttatttttaaataaacatttttttctggtgatacctggcagacattgactccgatctcatttgctagaattggatcacatgtccaagtctgaaccattcagttgcaaagagaatgataccgctatactgggtttatgccaagaacattacacatgtttgtggaatgctcatgtgtagacaacagtgtcttacacaacttcaaaaaaataatttatatataaatatgttttaaattactttttaaattcacaagaatttatggtatacaacatggtgttctatatatgtatatactatgctatacaacatggtgttctatatatgtatatactatgctatacaacatggtgttctatatatgtatatactgtggaatggctaaatcaagctacttaacatatgtattacctcgcatacttttttttttttttccttgagacagagtcttgctctgtcacccaggctggagtgcagtggcgctatcttggctcactgcaacctctgcctcctgggtccaagttattttcttgcctcagcctcccaagtagctgagattacaggcatgtgccaccacgcctggctaatttttgtatttttggtaaagacggagttttgccatattgtccacgctagtctcaaaattcctagcctcaagcaatctgcccaccttggcctcccaaagtgctgggattacagcatacttcttcttatttttttttttttttgcactaagaacacttaaaatttactctcttagcaattttaaagtatataatatactgttattaactttggtcactattttaattagacttaagatgtgtttgtattcaaattattttgtaagcatttaacacccaaatttgagagtggggtcagaatgttggaatttgatttctagaattagtatagggtattattttcctactttttttctgtgttcaataaaatgtttataagattcagcttcaattatattataacccatttagtggtgaatcagggaagaatgaaaataatttgataactttgttgccttgcatttatttaaaaaatttttaattctaggctaaaccctttttaaatgaaagtttaacttcttgtgttttcagatactgaatagctatgatacctcttgtgttgagaaaactttaaatttgcataatctgaagttatcttttcttataaacattttattaggtttacagtattgtctttttgttttgttttgtttttag

SEQ ID NO: 83 - RNA, 90 nt, Homo sapiens
  source 1..90: mol_type = other RNA
gtaagtaaaacatttttaacattagtatgcaatattgtacaaagtaggatagctagattcaacaagtaatatggatgtgtctttgtgcag

SEQ ID NO: 84 - RNA, 3663 nt, Homo sapiens
  source 1..3663: mol_type = other RNA
gtacatctgattcttattttgctttttctgactatgaaaaatttcaaatatgcagaagataggatggtatcaataatgctcatcacctgaattaatagttaacatttattaacattttgtcataattgcttcttctgatttttgtgggatgtttgaattgcagacattcctcccctaaatatttaatgtacccttttgaaaaaggcttttttctttaactaaccatagtaactttattatacctaacaaaatgacagtaattttctaatatcgcctaataccctgattatagtcacattttttacattttttgatcaaagaataagcatttggatgttacatctcataaatctttttaatatagaatccccttggttttctttttctccaaaaaatgtttgaagatgtatctaacttttgtgtgtgtgtcattttacttgttcctgtgtcccttgtattactaaaagttaggtcagaaccctaagttacattcaggtttaaacatttttggcaagaatacttcataagtagtgttctatactttatattgcatcacttcaagagtatctggttgttccatgttttgtaattgattactctgttaaggaaaagacaagcagaccaagtatggtggctcatgcctataattccaacattttggaaggcccaggcaggaaaatttcctgagcccagaccagcctaggcaatatagtgagactccgtctctacaaaaaatgtttttttttttgtttgtttgtttttaattagcttggtgtagtggcacatgcttgtaatcccagctacctgggacattgaggtgggaggatcgcttgagcccaggaagttgggggctgcagtgagctgtgatcatgtgccactgatctccagcctatgtgcctgtataacagagcgagtctctctcttaaaagaaaaaaagaagaagaagaagaagaaaagataaccatatacctccattattaagcaatttagctaactggtgatattttggtaccatacaaataacaaattatttgtcagtcctaatgattttagcatctgctgatgattgttgcctaacccaattattaaaagttgcaaacatcataattttctagttatattatgcacttacatttattaacagacatgcttttgtaaaataaatagcgtttcctcattagcccaggctatttgtttatcttgaagtttagctcctactacaaaggcaagataaatgcttttctctttaattaccagttttcagaatacacacttggtgtactctgcactacctgctttttttgtcccctccgctttctcttttttaagtatcagattagactcacagatttttaaatattccatgtgttttagttggagtcatattcttttgtctcaactttagccaaagagagtcctttaaagttgactcttatattgtcttgacaaaaattcattagtcttttgaacgaagcctcaaagcttgacttgttttctagcataagatgtcttagacttacctacatacttcatgcccatacttggaataaaccatttctttaaagagcccaggttccttttagtggggaaggcatttagataccaaaaactggccactgggcatcattgctctcagagtatcattgccactagtctctcagtagacaagttagaaaaatatgtatatatttaaaccatgagttcatattgttatttccagtttaattataacattatggggtaagtaaatagtatcggatttttactaagcttctttgattttgcacttgtatttttttcttacatagaaaacctttattattaacattaaaatatttgttttatcctacaatatacatacaataatttgaaaaataatacttgaattgatattaatagtaacaacaacagcactgctgccaaacatagtttaaagttttatttcaggtcttattttcttcagaatatatcttgctgagaatgtataggcaaagtattctacacttacttgaaataattgtcttcatgcggttatgttatacatttgatatatagttaggctcatttgtttttcattttttttattttagggatttttttcctttattgaattttaatatatacaatatttatatatgcaaaatatttaatcagagaaatcttaattctggtcttacgcctttcatattattctgctccaccctctgtaggtaacttattatctttctcatgtttcctttttggaaacataaacaaagacaagacaggttacatgacatgtatacccttctgcacctagttttataccttaccttgtagtttatttttaagcatgtaaatgttcaatgttcatgactaaatttggacaggatcataggaacacagaattcaaagtgaaattaaaatgggcttgggttctttactttccactttaaaggttgtaatgggtgatgtcaggctaataaacctattttcagcttgatctaaagcttaatactgagcatcaagaaattctttaataaatataagtgatatttattcagacatgtaataaggaaatgttcatgtcttatttttgtgttagatttttttagaatctacttttgttagagttttataaatacagttagtgtttgagatagaaagagaaaagaattagttttcttcctcttctacctgctcatgaacttgatttttttctcccaacaattgaagagccaagaaaaagggagattcttaagagatgggaaatagaatctcatctacccctgtttcccccagaacagtgaaactgaatcttaagggtaagatagaatagtgtgtacttaacttagatggagaagaaaggctgccaaaatgagatctgaagcgctattacaaatatttccatcgttactgtacttcagaatgaattacaaccgtaagtttttttacttcctcattcataaatttgattattccttataccacttctcagctttcatcattctttattgtacttttctatgtaatgtttgcctattatacagcaacttaagagaactgtaagtttggacatttcattttggtgttgataatagaatatctttgaatagttctatagttgatgagtagaaccatgaaccaagtaacttaaagtccttgatgttatttattacagagaactataatagaagctctcccgctaatgtttccatcatgtgtacaaaaagttttcttgttattaaagctagtccgtttaacttacaataagcataaatagctaagctgtgaaagttacctgtgataatgctaattttcccatttattaaaaggcaagttgttttccgatcataagaaatttagaaaagccatccaaagataaattccgagtgatatattcctgctgtttgttatgttttctcaaattaattgagttttattttacaatgacaggagttattaaagtattttatttttattatgattaagattttcaaagtaacatttcttatatgaaagaaattatgttaatgcatgtttttcttacatgggaaatcatatattttaaaaatgattttaaaattcgttttactttaagttgtattatctttctcaaaagtggctagtgcttgaccagaaaaaaagacaccagcataactcagtgtatctttatttacatag

SEQ ID NO: 85 - RNA, 5838 nt, Homo sapiens
  source 1..5838: mol_type = other RNA
gtaagtttgtgtgattcttgaaccttgtgaaattagccatttttcttcaatatttttgtgtttggggggatttggcagattttaattaaagtttgcctgcatttatataaatttaacagagatataattatccatattattcattcagtttagttataaatattttgttcccacataacacacacacacacacacaatatattatctatttatagtggctgaatgacttctgaatgattatctagatcattctccttaggtcacttgcatgatttagctgaatcaaacctcttttaaccagacatctaagagaaaaaggagcatgaaacaggtagaatattgtaatcaaaggagggaagcactcattaagtgcccatccctttctcttacccctgtacccagaacaaactattctcccatggtccctggcttttgttccttggaatggatgtagccaacagtagctgaaatattaagggctcttcctggaccatggatgcactctgtaaattctcatcattttttattgtagaataaatgtagaattttaatgtagaataaatttatttaatgtagaataaaaaataaaaaaactagagtagaatatcataagttacaatctgtgaatatggaccagaccctttgtagttatcttacagccacttgaactctataccttttactgaggacagaacaagctcctgatttgttcatcttcctcatcagaaatagaggcttatggattttggattattcttatctaagatcctttcacaggagtagaataagatctaattctattagctcaaaagcttttgctggctcatagagacacattcagtaaatgaaaacgttgttctgagtagctttcaggattcctactaaattatgagtcatgtttatcaatattatttagaagtaatcataatcagtttgctttctgctgcttttgccaaagagaggtgattatgttactttttatagaaaattatgcctatttagtgtggtgataatttatttttttccattctccatgtcctctgtcctatcctctccagcattagaaagtcctaggcaagagacatcttgtggataatgtatcaatgagtgatgtttaacgttatcattttcccaaagagtatttttcatctttcctaaagattttttttttttttttttgagatggagtttcattctgtcacccaggctgagtgcagtggcacgatctcggcttaacgcttactgcatcctctgcctcccagattcaagcagttctcctgcctcagcctctgagtagctgggattacaggtgtgcaccaccacaccagctaatttttttttttttttttttttttttgaggcagagtctcgctctgtcacccaggctggagtgcagtggcgccatcttggctcactgcaagctccacctcccgggttcaggccgttctcctgcctcagcctcctgagtagctggtaccacaggcacccaccatcatgcccggctaattttttgtatttttagtagagatggggtttcaccttgttagccaggatggtgtcgatctcctgaactcgtgatccacccgcctcggcctcctaaagtgctgggattacagatgtgagccaccgcacctggccccagttgtaattgtgaatatctcatacctatccctattggcagtgtcttagttttattttttattatctttattgtggcagccattattcctgtctctatctccagtcttacatcctccttactgccacaagaatgatcattctaaacatgaatcctaccctgtgactcccatgtgactccccgccttaaaaactgtcaaaagctaccggttacctgaagggtaaaagtcaagtcccctacttacctcatgtcatctagagcaagagatgaactagctgagttttctgaccacagtgttctttcttatgtatgttcttttgtacgtgctcttttctatatatagggaaccatttctctcttccagttgttttgctcagtgaatttctattcctgtttcaaaacttgttcaggcattaccttttttttcttaagcatactttttttaatggaacaaagtcactcctgtctacactagttctgcatcttatacataggttttgtacatagtacatatttatatcacatcaaattatatgtgtttacatatctgtcttccttaatggaatataagtcttttgatataaggaactatttaatttgtttctgtgtgttgagtatctcctgtttggcacagagttcaagctaatacatgagagtgattagtggtggagagccacagtgcatgtggtgtcaaatatggtgcttaggaaattattgttgctttttgagaggtaaaggttcatgagactagaggtcacgaaaatcagatttcatgtgtgaagaatggaatagataataaggaaatacaaaaactggatgggtaataaagcaaaagaaaaacttgaaatttgatagtagaagaaaaaagaaatagatgtagattgaggtagaatcaagaagaggattctttttttgttgtttttttttttgaaacagagtctcactgtgttgcccaggctggagtgcagtggagtgatcttggcttactgcaacctctgcctcccaggttcaagcgattcttctgcttcagtctcccgagtagctggaattacaggtgcccaccagcacggccggctaatttagtagagacagggttttgccatgttggccgggctggtctcaaactttggatctcaggtaatccgccagcctcaacttcccaaagtgctgggattacaggcatgagccactgtgcccagcctgtttttttttttttaaaggagaccagtgaagtttcaggaggagggaaagaaaatttagagttactagggagagagtgatgaagataagagatgaaagtggtaataagggaaatagcaaaatatcagggtaggtgggagaaaaagagatttgtaacaaacaataggattatcctgtgaaaaaggatgaaaggaagaaaaaaatggatagaaagatatttaaaacaccctcagcctcctgttttccctcctgtgtattcatagtatataaaactataattatgtactttacttaaaaaatatattattattaccttatcgtgcttatttaatcatagcatgtcctctttttagtctcattaccctgtttgtattattcttcataacacttaatacctgacattgtattatatattggcttattttccaggtactccactcaaatataagttctaggatataatttatttatcactgaaatccattgcttagagtacctggcatgtagtaaataggcattctgttttttcaaataaaaaataaaggaacttaagatatatatttatgttatatcgccagcctttttcctcacagctctattctgttgtacagaattacctactttacaattcctgtgtttcaaggggatctcaaatttaacgtgtccacaatgaactcctgatttctgtttctctcctagtcattcttatttcaatatatgttcagttacctaaccagctagtcaaggcagatactttagagttattctgtagtcattctttttccctaccatttttgttttccaaatgtaatttatgtgtgtcttcttcatcctcgcagctctaacccttgtccaaaccagcatcatcactcatctggagttccacaatgtctttctggctagtttccctgatttctctattgacccctttattctccacagtgcagccagaatgattgtttaaaacttcctccttaaaatctttaaattgttttcttttatacgttaagttaaattccagttccttgtcttggcatgccatgccctgcctggtgtggcccctgatggtctctccaacttcatgttttactactattgactcttatttttgcttactctgcttgggtgctccagtcctccaaatcatttcctgctccaatcatttcaatcattttttcctctcagatcttatagtattccaaatgctttcttcctttggagcatctgggtttactaataaatacttcgtacctcacagttcagcttaaatatcaattatttggtggttaagacatccttcaaccgctctatctaaatgttcctttctattattcactggctcagtactctgtttttattttctttctaaatgtcaacttttttttttttgagtcagggtctcactgttgcccaggctcgagtgcagttgcacaatcatagctcattgcagccttgccctcctgggatcaagtaattctcccacctcagcctccaaaatagctgggattacaggtatgcatcaccatgctcagctaattttttgtgtttttttgtagagatgaggtctcactttgttgcccaggctggtctcaaactcctggactcaagtgattctcccacctcagcctcccaaagtgctggggttacaggtgtgagccactgcacctggtcgatactgacttttttttttttttgagatggagttttgctctgttgcccaggctagagcgcagtggtgtgatctcagctcactgcaacctccacctcccaggttaaagggattcttctgcctcagtctcctgagtagctgggattacaggcaagtgccatcatgactggctaatttttgtatttttagcactatgtttagtactgtgttggccaggcttgtctcgaactcctgacctcaagtgatccacccacctcagcctcccaaagtgctgggattacaggtgtgagccaccgtaatcggccaacattgacatttttagtagactttttgtttgtttacttgcttattatctgctgccttccacactctggcgaaatcctgccacccacccacacacacataggcactgaatgggcagaactctgaaggccagaattttatatttcttttcactgtaaacatcatcatctgtcactgatggcacactaggatgctcagcaactgtgtgcatgaaggaagtaagcactagtttgtgaaggctgcaaaactcttgagtattctaagagttttggccaaaatgaatgtacagctttagtggcagaagctaatactcagaaattgaggccgtatattggataacacaggatttggatgattattttaaaataatattttacattgtatatatgtgtgtgtgtgtgtgtgtgtgtgtgtgtatgtgtgtgtgtgtgtatatatatatgtatgtatgtgtattagtccgttctcatgctgctatgaagaaatacctgagactgggtaatttataaaggaaagaggtttaattgactcacagttccacagagctggggaggcctcagaaaacttaacagttatggcagaaggggaagcaaacacatttttcttcacatggtggccggaattagaagaatgtgagccgagcaaaggggaaagccccttataaaaccatcagacatcgtgagaacttactattatgagaatagcgtgggggaaaccacccccacgattcaattacctcccaccaaatccctcccatgacatatgaggattatgggaactatgattcaagatgagatttgggtagggacacagccaaaccatatcagtatgtatatgtatacaagtattatatatatatgtatgtgtttgtatgcatacatgtattatatatggaggaaattctaattttgtaaaaaactggattgtgagttttaaggagatgttatataaagttaagacaatgtcattttgtggtattggtctgaattacaatgtagtttcttagtgatatttttcctttattcag

SEQ ID NO: 86 - RNA, 2912 nt, Homo sapiens
  source 1..2912: mol_type = other RNA
gtaagttttatgactctgataatataaaatgattaacatctaataatgaatatttcttatttaaagttccttttttatgctagattaaaaggaagtattttgactaaaaaaagaaagaactttctgcctaataatttaacttaggcagatgaataatcctgtacttaaccccaccaaagtttagttttcagtccttaagttagatttgtttctaatgaaatcatatatgttaaaaatttatgactaagtattagctactttgaaccgtttaacaattaaaactgatgatattttattaatggtattatgagttctttcactgagtgcaagttatattagttatatatcacttgatatttttaaattaaaagataccaggaaacagcaaagaaaatgtgaaaagaagttgtatttctcatagttttactactatattactgtatatttttgctcctatatgcttacatattttatatattttaaattattataaacatggttttatactgtatttagatagtaatatcaaaaatatttttatggccggcgcagtggctcacacctgtaattccagcacttgggaggctgaggagagcagatcccctggggtcaggagttcgagaccagcctggccaacatggcaaaaccccatctctactaaaagtacaaaaattagccaggcgtggtggcagttgcctgtaattccatctactcaggaggctgaggcaggagaattgcttgaacctaggagtcagaggttgcagtgagccaagatcataccacagcactccagcctaggcgataagagtgagactccgtctcaaaaaaaaaaaaaaatttgttttattcatcatacttataaatacttatacaatagcctaatgtgtttgagtgattaaatcactagctttttatatttttgctattgcttatagtgccacagtgaacattttcatgtatatctaacagagatattactgtctcagaaggtattgaaatctttgttgctctcattagagttttccatattaatttttcaaacagttatatagtttataagattttcataattttatctcatatattgtgcttcataattttcaaataaatttgctgctttcgataatgtattttcatgtatttgtttcctagacgttagagctattcaaggtttttattactaaatagagctgttctcttaaattggtaatgagatacttggtttagagaagcctaacactgggaaatcttacataagctacttttagaaatgtaatttttagctcaataagagattaaatatgaattgacttttgtgtagtatttgcatggaagaaggtaccatttaaatgaagacatgagagtattacgtacaattttagtaggttctttttattttatcatctttatttttaataaatgctgaattccctacagaaattctttaatttttacatatcttgatctctttcatatatggatttatatcaccgaagttttaagagtgtttccctattccctgttgcccttatatctttgtttaaaaatgtcacatcattagctttttttcatctaggaatttgttagtgttgggctgttgtgctctaccctctctttaagaaaactccaaacccaaaaacatacaagatggctagtctgcttcagcctttgtgatgtgcttttctcttctaatcagagtttagcacaatacagaatggagaaggactcctttatatattggtatttattgcagtatttttctacatggtgcctaaggttacttgaatgagtctttattccataatgaactgatttactaatgcttttagcacctgttagtgatccattattgttagttacttgattactgcttgccacagctattctaaaataatacattttaaagataaatacagaacataatgaagtactttttaaaactgagatagagaccaatttttttttcaggaaatgtatattactttgagaaaactcagttataaaacttgaacttatgaagctggaaaaacaggagggggcattattggtattgtaaaaggctgtttacaaagtgagttgctgcttagttcctttaagtaattggctaccctaaacacatcagttttaagttgctgaaaagcaaaacactctaccaaattttgttttttttctagaccatgtttacaaagcaaaagtatgttttcttccccccccctcaaaaaatgactaatgacactcctatgcgatgcctttttatggtaaattgaggcttttagttctctttccatttagccacagacttttgtgtccaaagacaagctgcgtaactgcatatataaggttaaggcataactactaataaaagaatgtaaaatatttgatattaggtctgtacaaagaccaaataatactcatgattagacaagattatatttggtagaatctatccatcatatggcttcagattttacttttcagcttggctttgtgagactttaaaaatcaagtcattgcacttatattcacaaagtcacattgctttactgcattgcttctcatacagtttatctcctttcagtaaaatgtttacttgccatttttaaaatttcttatatgtgacacttctacactaagtcctttatgttgttagttccacaattctgtgaggaataggtttttttttttaatcatttgattgatgaagaacattaagttccacagagattaaatggtacaggcatcacacaggcaggaagtaacagagctaagattagagtccaggtctgatggaattcagaaagctaatgtgctttccatggaactataatgctttctaatatacagcatctaaaatatctgaggtaattttaatataaacagcatgagattgacttaaatattattgcatgtag

SEQ ID NO: 87 - RNA, 937 nt, Homo sapiens
  source 1..937: mol_type = other RNA
gtttgatattataagttttatcatacaattatagaataaagaattagttttggtagacattgtattattgttaagtggtttgtctggatctctgaaatatcttattaatatagtgcctatgttttgtgtaataaataaataaaagatttaaatctgaattgtttaaaaggaaagcagatatttctgtaagtttttctcaccaatgttatattattagatttaatttatgaaatgttatttactaaacaatggaattgcctttcaccaccatcccttcatttaacaaatatttattcattgcctattacatgtcagaccctgtgttgggactggcagtatagcaagaaacaaaatagacaataatctctactttcagggactttacattctaattggtggttttatatatttttgatgtggtcagaatcattaaactgtgtggcagtaaatatagtttgcaagtatttaacaatttatgattaaacacaactcttacagtgtttgcttaccttgagatttaatatattttcaaagcatttatatcatttttgttttaactatgtcactaaatctatatgagtaagattttattaactcatttggatttatttatagatgatacaattgaagtaaaatataatgagcagattgcattctaagcaaagtaagaatattgcaagttcagatattattagataatgagttgcctaataaaaatgacttttggtggattggaatataaccagagtttccatagtttgtttctgattctttcatattttttaccctccttcagtctgttcttaacacttcacacttaatataatatgtgaactaaggccaagtaaagaggattgcagtactttaaaagctaaattacaaagaaaacctcaccaaaaattgatgtatctgaacattttttgttacatttccttag

SEQ ID NO: 88 - RNA, 1841 nt, Homo sapiens
  source 1..1841: mol_type = other RNA
gtaagtgcatataagcattttagccatttgactagatgtatcttctttaatttgtctttaagaaacccaattacaggtatacaattcttagtagtaattgatactgatttctttttataagaacaggattaagtaatattaagatcggttttaacagggttaaataataatattgacgagaataatattgttaaagaggaagtgacctctcaagatttgcattttttagagttcaggaatattattgcagaaaggtccagttcctccacatattgattttttggggaaggggtgatggaggaggaatggttgtttattgtatttaaacttaagtttcttcattttaataagggagtaatagtacctcttctacctgtttcataaggttgctgtaagaatataataaaaaattcagattttgatttagtttacatttatcgggcatctactatgtactagtcacggtgcaaggtattaaacatatattgacttgtacaattatacttaaccttgaggttatatttttgttttcattttacatgaagaaatatgcccagctagtttagaacacaaaatatatataaggagtaaatactgcgtgctggctgggcgtggtgacatgtgcctgtagccccagctactcgggaggcagaggcaggagaatcgcttgatcccgggaggtggaggttgcagtgagccgagatcgcgccactgcactcctgcctggtgacagagcgagactctgtcaaacaaacaaacaaacaaagaaaaacaaaacaaaaaaaccgtgtgccagctatatgctgtattttcattctcttttgtaattaggtgatatttcagtagaaaagtataaggagcacttagttaatctgtcaagcataaatagtaaaaatattttatggcctactcataaaaatataaccattcctttggagccttgatagttctcttgggaatatcagtttttgacatctttttcactatgaaagacccttttttttaaaaaaattgatcctttcttctcatggacctcttttgatataaactaacttataatagttcattttaatcatattttgttaatcatgcaactggcaatgagagcctctcatcagtatgaggaaacctgccttatctataatactgaactaaaattattctaacccaaagcaaagaaactttacattttgctttgcctgtattagcttatcacagtattcatgagggaatttgaaggacttattaccattaggctatctctttttttttttttttgtaattttattaaatgcatgttttgtttcttttcacattactgataacttgtagattaaaacaaatcaaaacatgcattaatccatctaaggatcctagaaattttacatttctgtgttcttaactgtgtgatggtcttagataaatgtactaaataccttatcctagcatattccaaattatgacaataaatgttttatggaaaaaagtatgggaacagaagttctttggctatatacatttggaaaatactatatagtaagtatgatttgagataattatatatgatagaacctctgggagcactgaatatatgttaggaatattcaagagggaggagggatgttgagaatgaagttttttttatatagcaaacatgataacctctgatggaattatgtttcatgaaacagtttaggaaatcctgttttaatatttcatacaaagaagagatagatgctgaaaacgaatggctttttgaaaaagggtctagaaattttgaattttggcatttacttagaaagtgtacttaattgttcctgaaataccttatcatttcctag

SEQ ID NO: 89 - RNA, 1240 nt, Homo sapiens
  source 1..1240: mol_type = other RNA
gtatgtgcagtattggctcttctacatagaatccacttttttccctaaatttacattagatgttgggagtgggatatgttatactttttgtttgtttcgagatagggtctcattctgttgcccagggtggagtgcagtggtacattcaaggctcattgcagccttcaccacctgggttcaggtgatcctcccacctcagcctcttagacagctgggactacaggcacgtgccaccacacctaatttttttgcattttttgtagagacagggtttcaccatgttgcctaggctggtcccaaactcctgggttaaaatgatctgcccaccttgacttcccagaatgctgggattacaggtatgagccaccatgctgggccattgttacatttttaatcaaaagatataccaaccagaggctgttattcttgttagttggaacctgattagaaagctctttaatttgaaatattgttcagtaatccagtacagcatttaaatgcctatagatgaattatgctgctgatcaaaattaggacactgagaattgtagttagtaaatctttaataacaatattttctcttgtatttatatgtaactttttacatattcttacgttatatatgttgggaattataaaaacatacacattgtcctgatcagtattatgttacttgcaatggaggttaaaaaaaaactgtaacagtcaggcatggtggctcacgcctgtaatcccagcactctgggaggccgaggcaggcggatcacgaggtcaggagttcgagaccagcctgaccaatatggtgaaaccccgtccctactaaaaatacaaaagttagccaggcgtggtggcatgtgcctgtaatcccagctacccaggaggctgaggcaggagaattgcttgaacccgggaggtggaggttgcagtgagccaaaatcacgccattgcactccagcttgggtgacagagtgaaactctgtctcaaaaaaaaaaaaaaaaaacaccagtaacatacccactgttattcagttacatttggattttaagtttgtttgattctaggttttttcttttacagttctttggtaattatttgtattaaagcaaagttacatttttgtagatctcatgtgccactgtgttaaaactttgcttagtaaattgtgaattttaaatctgtgataactttcactggaaaaatttgaaacttactacaaatatatattttttttaatatcag

SEQ ID NO: 90 - RNA, 1087 nt, Homo sapiens
  source 1..1087: mol_type = other RNA
gtaaacatttaaacttgatttttttttttaagagacagtatcttgatctgtttcccaggctggagttcagtggtgcaaacatagctggaactcctgggctcaagggactctctagcctcagcctcctgagtagttgtagctggcagtacaggtgcacaccaccatacctacctaattttttaaaatttttaaatttttttgtagagacaaggtctcactttgtcacccaggctggccttgaactcctggcttcaagtaatcctcctgctttggtctctcaaaagtgctgagattacaggcatgagccactgtgcccagccaattttaaattcattatcttcaaaagagttacatgataatttcttaatatatgcctatatgaaaaatgcttaagatacaaattccaattatgattcattaatttagattttataacttagcagtgttggctatttgaatgtctattatacgtaaaaataaaattaggcttttctaaccaaagattttagtgggaatgttcagattgtataatagcaaagaattttaattactataggaaaatttatattaattaaacactaattattatatttaaacattgtagtagttatcagttgatttctactgttcataattatctttgatctacaagtagtgggcccacatttacttttaatatggtttaatcttcatttagaaagaattaaatgaaaaataattatcttgcaactacatcctgttctctaggctagaaacatttaggatttctgtttttgaaagtaataccaaagttccaatgacctgcttatagtcagtgttcaataaacgtataacaaatgaaagtgaatattagtgatgtccattccaacataatttgaagatttttattgtaaaatcccacatatttgtagaaaagtctatggaaatcctaaataagattttgtcatgtagtttgacaaaagataacattgtgtcttattttattttagaatggccattactttcaattaaaatcattatcatcaatggaggaatgttatttgttaatatagcatttatatttgtgtatataaattgtaaatcttag

SEQ ID NO: 91 - RNA, 1281 nt, Homo sapiens
  source 1..1281: mol_type = other RNA
gttttattttatatttatttcatttttttccctaagttttttttttttttttttttttttgagatggagtctcactctgtcgcccagactggagtgcagtggcgtgatctcggctcactgcaagctctgcctcccgggttcatgccattctcctgcctcagcctcccaagtagctgggactacaggcacccgccaccgtgcctggctaattttttgtatttttagtagagacggggtttcaccatattagccaggatggtcttgatctcctgacctcatgatccgcccgcctcggcctcccaaagtgctgggattacaggcgtgagcccctaagattttaaacaagaatattgcacaaatgactatgttatccttctaattaagtgcaccttccattactaattgattatataataatttgttttttattttctaaactattctaaaaattcatatttatttagcttttataacagtagtcttaatcttaaaaacggcaatacataagcaacctcatttggtaagttaatttttattttgatattggttatttgacttttcacagttccacgtttctactggctctcactgatagagtaagaagtcagcttcttatagaataaagtatatacttcagagacagatgaaattcgtcaaacatatgactgtctcagagattgttccccctgcttaaattgttcttaccctagatacctttggtatttacactgtcagtgcctgcaggtcttagctcaaatgtcttaccttatcagtgtatccttcaccagccacctaatatacaacagtaaatcctactatccagattcctaaatagagattaattaacttaatttttctccaaagtgcttgtaaccttctgacgtattacatacttactggtttattattgactgtctttccttcgccagaatgcaagttccgtggtgacacggacttggttttgtttactgccatgtttgtatttcctagaatgatgcttggcacataatatatgtcatcaaatatctttcgtatagctgaacggatggatggatggatggatggatggatggatagactgaaatccttacttcacatctgcctttgtgatcttacacaagttacttcacctctctgagtttgtatttttttccataaaaggaaaataattacagtttcttcaatgtgttgtgaggattagataagaaaatatatataaaatgcctgttatgtgcctgatgtcttcgtgtatgtgtctgacacaaattgtcctttttttag

SEQ ID NO: 92 - RNA, 217 nt, Homo sapiens
  source 1..217: mol_type = other RNA
gtatgaatgattaatcttgtttgttactctgtagcatagtctagagtgttaactcacagaaatatttcctgtatcagatgtaattttaattgatgttatattgtatatttaaaatataagaggggtttaatctatgttttatcatacagctgtaaaaattaatagttactctcaatgctgcaactgcttttttaaaaaacatactatttcttaatag

SEQ ID NO: 93 - RNA, 1186 nt, Homo sapiens
  source 1..1186: mol_type = other RNA
gtaattagaagaatttgcattttgattagtgtattatttggtatgtttggggggctttctaaataatatttctttatgagggcaatgcatagaatgatgaatctattgctaatttcactatttttctattctcctataatgtttctaatagccaataatgaacagcagatatagttaatttgaattcactatttaattattagttggtacctttcggtacactgaatatgaaaggaaataaaaagcatttaattgtagttctatgagcaatatattctcttatatgatctctttattcttacttttttggttttattttgaagtgcatgttacataatctatgaatcaattttcagttcattgcctttaatgcatggttaaagggttgaaggtaaattagaaattactttctgttttaacctagatcttgaatttgattagtaggtgatcaaatctgtcatcttcattaaattattcagaaaataatgtaaactgaatgtgttttcattttagttttcatctaaataaactgcaaatacatttaaaatatacataaagaagtttttcaagtaaaactgtacatttttaatcatttcaggaaacgtagattttcttcagtaattttaagatttgtcatttatgtgaattgccattgaattacttaatttaaaatactcaccttaatcctcttgaagagtaaaaatttttctgtttttttctctttgttttaataagctgcggattttatattcgtaatttattgagttgggcctctaaaattccagttttgtacttaactgacttatagattagtctcctaatgctctgctagtcaatggaccaaaataaaagaaataatttattacatattcttcctaaatctagtaccaccatacatgtataattctaaactgtaatatctcaataaagtaccttaattaaattttatgttcatcataacaatgaagtttctagcatatgtaatagtcttataaataagcatgcaaataactgctgtcaattagaattagtcagtttaaccttattaagtatcaaatggctattgtacatatgatgtgaaaaataaagtgaattttttttggctaataactaatctaaaattcagatgaagcattttaaagggaaaaagatactttaatgatttattataatttaatcattgcag

SEQ ID NO: 94 - RNA, 631 nt, Homo sapiens
  source 1..631: mol_type = other RNA
gtattttattatattatgagttatgctgttatccattagtttttttaagcaaatgctaaatattattttaccctaaagtggtatttcttttcttgctttcaaatgattctatttaagaattgttacttgcatgtgattggattacacctctgtcagtaaaactggaagtttgtgtacatgtatctttctattatacactgactaaaccacgagtagctatcatggtgaaatcatatgattttgaaaaatattttaattgagtttataggtgaggattgaggcaatagggtggaatgaaatatatcacaccggtaatcagtagaaatcagatttgttagaacttcgtgggggaaagctaacatttaattttttctagaagtaagttaaaagatgatagatacatgtcattctaatgttaagaataaattatgaactgaggctgggcttgtcaacttgaacattgtctgaggggacatgcataccagtctagatacatacatatatggagatactgtttcttcctcatctcaaaggaattttagaagattgaagagaaaatatataaggtcttcaaaatgtgaatttgttttaatcacaatttaagatatagtttcgattttctgtaaaacag

SEQ ID NO: 95 - RNA, 616 nt, Homo sapiens
  source 1..616: mol_type = other RNA
gtaagattctaagaactttgttccattctttattgatttttgtgaccatgtaaattaaaattcagctctcttcttttttggaatggaagttaccctttttgttgccaaaataatcttctgaaaacatagctctgatcattcttcctcctgtagctcaccgctgttcacaaaattatatttataattcttagccatgtactcaatctgctatgaacctacctgcctttcttttcaaattctactcactgtgagtttagctatatctaacttccagaattcagctcatatttgcctcttttgaccattctgttccatatgtatgaaatgacatgtctttcatcttttaatgtgtaaccttagcatatttgagcattacctcgttaattcggtcaacacttattgatctcctgctacgtgcagacattttgctagctattgtaaatacaaataataaagtctgcatttcctgtcttctttaagccttcattgcctattaaatcattacattttagattagatattatattttgatcatttgaggaaccaaattaaaaatatggaataagtatggcattgaattatacatgcctattgctaatatattcatattttatag

SEQ ID NO: 96 - RNA, 2635 nt, Homo sapiens
  source 1..2635: mol_type = other RNA
gtaagtcttcgaaatgtattgtaaaaataggcaaatgataagtgatataatgaagataaacataagtgtttgctatgccaggcactgttctaagacttttaagtatattgtctcatttttatcctcaggactgctggttacatatgttatcattttccccattttaaagagaggatatggcctcaggaatgcttaatagcatgtctgggggtagatgggaaagccataatttgaaactagtcagtctgactcaaaagccaatacaaattcttttccagaatctcatttttaccttctttgagcctcagtttcatcttatttatttatttttatttttgagacaaggtctggctctatttcctaggctggagtgcagtgacataatctcagctcactgcaaccttgaccttccaggctcaaaccatcttcccacctcagcctgcagagtagctggcactacaggcaggtgccaccacacctgggtagtttttttgtatttttgtagagacaaggtttctccatgttgcccaggctggtcttgaactcgtgagctcaagtgatccgcccacttcggcctcccaaagtgctgggattacaggcctgagccattgcacccagcctcatcatctttaaaatggaaataataatacttaccctggccctttcagggtggttatatgaaggtcaaattataccgtgtatgaaagtaatttgaaaactgtaaaataacatacagatagaaaacttttgattacacacttataagagtgtctgtcatataatagagattctaaacattgttcaaccactttatcagaacgtagattttaaactcaaaataggtttatagttaggtagtttctaatcattataatattatctctatgggcctaaattttattatctgaaaaaacatgagaaaattgaactgcttgacttataattccatttcagctctcaagcccctgctagagtctttgattctttactcacttattcaaatgcctctgacagaattaacactatttttgctttgctaaggagctgccactgttaagaaattactctctaaaagaaagaaaattggcaacagcatatgtgtattttcagtctcttttcctcactctattaaattttgtacaagagatgttatttttggtctagtaaatttctgtcatgttttggagtataaaattacttgtgcttttgcatctaatttgtgggtgtagaaaatcataatcttttgaaataccttatataatacatttttttgccacaggaaatacttgaagttattgttgtgtaccttacgtcattttagtccaaaattatacttgtgttctctgtgtgcatattttgatatgtattaggagattatggatctgtgtgatttcttaagtaaatcctgatattttcacaatttgatgatgactctttaaagttagacttaagttttgccaaaagcaagaagcctcaaagagtaacatttgttcatgtcttaacactatctccctcttattggtcagaatctcagtatggatgcagtgtccatatgcacaacaatatattaattcagtttaacagacttaatgctgaataagcaataagattaattgaattaactaaatcttttgatagtatccacttccatatatatagttatagatataatgctagtgaatttgaaccataaacaaattaataatacatgtgatttctgtgaaaatttatattagtcttttcaatatgtcaatatagggcagtatttctcaaatatagaggatcagtttttcaccattgtccctcttggggacatttggcgatgtctggagacatttttgattgtcatggctcgggggtgctactggtatccagtgggtagaatcaaaagatgctgctaaacatcctatcatgcacaaggcagccccaccaccaacaaagaattatccagtcaaaaatgttactagtagtatggttaggaaactatcatatagaggaagcaatcacattttacaagagccataatatttaaaatgcctttttgttcattctctgtatatttgactagagtcacaaaataacttgataagattgttgccaaaaatattagaaactagaagaaaaatgtgttgttaagtctaagagtagttaaatgaaataaagaattattcttctttggatttggatgcctgcatcaagatttagattgtaaggatacttaggactgaacatttgctctatatgaaatttgtattaatcaaggtatgaattgcagcaaccactctattaattacatatgtttggccaggtgtggtggctcacacctgtaatcccagcaatttgggatgccaaagcgggcttatcacctgaggtcatgcgttcaaactggcctggccaacatggtgaaaccccatctctactaaaaatacaaaaattagctgggcctgatggtgcacgcccgtagtcccagctactcaggaagttgaggcaaaaaaatcacttgaatctgggaggcagaggttgcagtcagccgagattgcgctgctgcactccagcctgggtgacagagtgagactgggtctcaaaaaaattaaaaattaaaaaacacacacacacatatgtttatttacatcag

SEQ ID NO: 97 - RNA, 952 nt, Homo sapiens
  source 1..952: mol_type = other RNA
gtaagtaacaacagaaaattatcaacatttaggaaaaatatgtggtagattgcttttagagaagatttgtaaatttataaaagatggtagtataaatctccgtgttgtaataaaaagtatgagctttatcttatgctgttaaacaaggtattttagacaatgctgtttttgtgggcagatatagtccaatttatctttttatgttttcgtcaatctgatttgtgaattatctatatgaagttaggaaaaatcttaatgtacattacaaaaatataatatatattacattgtattttctttttttctactggaattttatgctactgaggctatttttaacaaatgaacaattttgaacaatttgagggattgagggaagtatgataatgacaaaaagggatgaaaaaagggggtcatagagatgtttttgtgagaaggagttggtcagtgtattctgatttattagggttttttttagtttatctcagatttgatctatttaaattgttttagaagatgctggtgtttttctgtgctagctatgaaatttatgggtaaactttaagcctttcctagtccttttgttgtctacctaaattcaattaatttcatatggaaggatgtagtaagtgagtaatataaatatctaaaattggatgtttgaaaacaaaacatacctgttttttgtaatagcttgatttaatgctgagttctcaaaatcattattaagattttgaactttcacattcaatgtggaaagaattgagtgtaattacaaaagatttatttgaaaaagttgagttgttaatttgtgaaatatgttccattaaactcataatattttagaaaaatagtaggaagtaataaagcttgtttattttttatatcatatattcatataaaatgtcagttttcctttaaaaattacattttttttttggttaatttttag

SEQ ID NO: 98 - RNA, 1173 nt, Homo sapiens
  source 1..1173: mol_type = other RNA
gtgaacatcaacacgtgttaatgtaacaaaatttctgataattcctattggaagagaattcactatgatatatagtaattttgttgatgaatagggaatttataatgcactgttggtggctagacatagacacacacatgcatttttcaacaataagtctctttatgatactcatttactgattatcatcttggggattaggaaaggataggccattatgaactactgtttctaatgaaattaaatttaagaaatattttacttaggattttttttaagactttattatttttttagagcaattttaggttcacagcaaaattgagaggaaggtacagagatttcctgtatatctcctaccctgaaagtggtacatttgttaaaattgatgaacctatattgatacatcataatcacccaaagtccaagtttacctctattttagctcttggtattttacactctgtgtgtttagacaaatgtataatgatatgtatccatcattatagtattatacagggtattttcactgccctaaaaatcttctgtgcctctcttcttcattccttcctctgcacctcaccaaacccctggcaaccagtgatctttttactgtctccatagtttcaccttttccagaatatgttatagatggaaacatacagtgtgtccccatcattctcaccataggacagctaggaactcctttctagtggcatacatattgtctagtattgtaagttacccttttatatcttatctttgtaaactaggttagaaattacttcaagtcagagatttgttctgtactactcttatgcttcatagtgtttaaaacgttgtcatatatattgttatatacttgtttgtttaattaattcagccaaaatgaaacgtgcatatttgataaaattttgtttgtgggtgtttgttgaagatgaattgctttacactagttttttttttttttctcaaagtcgacttttttcctcaaggtagacttgacatgaatatggaaaaatatatgtagtttgtggttattttttttctcttgtgtacttaaaaattcagactgaatttttcttataatggtatattttctgttttatgttccttttatcattgatacttcttgaagagtcatgaataatacctttctttttctcttattag

SEQ ID NO: 99 - RNA, 352 nt, Homo sapiens
  source 1..352: mol_type = other RNA
gtaattttatatttaactctgataatgtctgatttacaatatagaggtagtagtttatttctactttatcattttatctatggtatttgttaaaactgactttcaaatcactttgattaatgtaattaatttcttttgtgacttctattgtgtttatagttctagagtagcatattagtatgttgtattaaaatgcagaagcagctaccagattatcttatgtattaagtgtcatttagaaagtatggtcagtgatagcttcagaaagttgctattatataattgaaatatttactgtctattttgttttacatttatttgtaaaaatataaagttacattttattttttag

SEQ ID NO: 100 - RNA, 5295 nt, Homo sapiens
  source 1..5295: mol_type = other RNA
gtatgtgaagaaacatactgacttatatgcttaaggtagtgacagagtaagttaaatacatagctgattaacagttaatatactgccttaatttgatgacctggctgtattaattctgtattaattttgaggactataagcagtattgaataacgtagaaaagtctaagtttctgttctgtaggaatttagagtctacttgaggagatacctataatgtaactcttatttggaaattactacatcaatttcattcatctttctgacattagagtacctctgaagttccttcacaccttaacatattcaactgtgtatcatttctctccaaagtaatcatttacacaggttggtgcttttgacttttgggacagaaagatagacattttaagataccccactttgacccaaataggtcctttttaatccttcaggagactaggctgttatttcagatagcaaagttatttggaatatcttcagtatttgcagtaataatcagtaaccaatctgctcatagattaattctgtgggagaaattgcttaaaattttatagttcatagtaaactgttttgtaataaaaattactgattgaaataaccccaaaaaaaactaaaattggctaaaatgcgtgtaattaaatttgttatggacaataaattggagataacttgttggtaacattcaaaatatcgaaagtgaactgggaaatgttgatgttagcagtaatatttgccattgaagaaaatcagtatggaggagctatggttaggaaaatttttattataaaatttacccagaaaatatttaatgtctataaaataatttcaatcacatgaaaatggaaaagaaaattctgtctttaaaggcattgaatagaaaataggtaatggaattcaaatttcttaatagagtatgctcccaaaattattttctatgaaaattcattaatgtcagtgtaatttattgacactatttgcgtggagtcacaacatgcttgctgtcagaagctttgctggtgaaaactgtaagatcaaagtgtccttaatcttttggatttccatctttctaactccctaattggggataggcctgatcttatccctaaatggggataggttagaaactggtatgtttgttcctaactggtgtgtttctataccagtttctaacctgattcctatcagaatgttttaagagccttgtggctttgcctggactcttctatgctacagtttatttagtttatttattcagtttattcctccttaaagtgggaataatactatctgtattgccagtttctcaggattattttacataaaatgatatgatatgcggaagtcttttgtaagccatcacatccatagcagtataagatattactactaactagaaagagaaaacaggggtctatgcccagtattaaaattggcattcaggaatctagtgagaatattttttcaggttcattgcttgggcatttctaatttatactcaagaaatgctttcatattgtttggaaattttagtacccttttctctgtaaacagaatttgtagtctacctatgtaacaaaacccacccctgtgccttgcatttcattctccttagcatttattactatcttaacatactagacatgtacttgtcttttgttcatcttttttttttctttttttattagaccataaactttgatggcaggaactttgcctattttatttattattgtattcccagcacctagaacaatcgctggcacatagtagatgctcagtatttgttgaatgaatataaatttttaaatgttataataatattattctgaaatctatgcatacgaagcttttggtacagaaaacatgaaaagagaactactgccttatcatccagtcttcttccctcttctcattcagtctagaacataacctgttttggaaaaagttctcaaaccatatgtttatcttgccctcaaaccataacaacaatcaatgcaaaagacttctgtgacccccagaatatgtggggatttctccacatcagcaagcaagcagttggttttgtagcagacaccaactgggtgtcgtccaattcaattcatcatctacctggagatagtgtcagatcccacagatatcttacttcgatcaaatcacaagtccaggcctccgtgacttccgaagttcccacatccccagcccccagctttgggtttgattaatttcctggagtggctcacagaactcagggaaacatttacttacatttaccagtttataataaaggttattacaaaggatacaggttaagagatgtgtaagaagagatatgggggaaggggtgtggaccttccatgcctttctggggtgccaccttcctctagaaacctccacatgttcagttctccagaacctctctgaacccagtcctcttggtttttagggaagcttcatgacatcagtatttcttctcctagggtatggggcaggaccccctcgtattagggttttaagacccacagtcagaaaggcaggggaagattacagtcctgccttagggcaggtgaaaggaggatgggagaaggtcagagagactcttttctgaggtgtgctcggaaggcctaacacactcaatattataactaaagatgaggacaagggctatgagagttataagccaggaaccatggaaaaaagcctatatgtaataacaccacaatacccatggtaccattcacgtttgttgtttttctgtttttcaattgttctttcagtcttggttcccttaatcttaatttagcaagtaatgccaggtgggataaaattgcccaaacccaacaaagtactgtgtgctgcaggattatttaatgacataccttatgtcccccactagtatttacatttctgggagtacagaaaaattcttgtacatatttcagaaaaaatgaaattaataactatcaaccacttagtgaagtttttacttttttttttgagatggagttttattcttgtcacccaggctggagtgcaatggcgcaatctcagctcactgcaacctccgcctcctgggttcaagtgattctcctgcatcaacctcccaagtagctgggattacaggtgcctggcaccacgactggctaatttttgaatttttagtaaagatggggtttcaccatgttggccaggctagtctcaaactcctgacctcaggtgatctgcccgccttggccccccaaagtgctggattacaggtatgagccaccacacccagactgaagtttttacattttttaaagggcacttattagctgaattaaataaggtaaaaaattgactagtattagagacaagaattggagaatatagttctctagtattcgagaaagtcgttttgataggacaactaatcttagtgagaatttggctttatttcatatttttttaattttttgagatgacgtcttactatgttgccctggctggtctttgaactctgggctcaaacaatcttcctgcctcggcctcccaaagtgctgagattataagcatgagccatctccccaggaatttgactttaaaccatggttctcaaccctttcagattcaacattccctttaataaaaaatataatgtttcataatttcccctttactattataattgaaatgcatagttaacataaactctacctacttacataatttcaaaaatgtcattatgaatgtcctaaatgaaatatatagggggaacataaaaggaatattcatatttcaacatgtaaatgctttggcatgactccattggaaaatataatgaactagtcatgtgcttgcaccttcattaatgtgagttcaaagctacgattgcagactgacacaaatgtgttctattggcaactgatgggtcatgatggtattgccatttgtaatttgatttccaaaatggtaaacaaattgttggtgcagttctcagcaaaacaatgtctataatcttaccttttataagactgttgtattcctagaaaacttagtgtatagtaaaaccattaaaaaattacttagtgtgaatatgttagttggagataaattcttagctcagaccagtgtaagcagaattttttactgtattaatatccagtagaacatttgaaagttgttcagtgcatgagactattctgcattggataggctttctttggctcctttatcatagttataataaaccatgacacctacccctgaaatgccctaattcccttccgtttctttttcttttttctttttagcacttaaaactagctaacttactacaaaatagatttagatttatttcttgttttgttatctgtatcgtttgctcccttctccccaatctatctaaccaactagtataaactagatagtaagattcatgaagatacacttttttatctgattttattcatttgttctattcctattgcctctagagtagtacttggcacatggttagcactaaataagtacctgtcaaatgagtgaagtaatgtgcattgaagacttgaaggggctctgatgctaggaaattgtcatgggataatagatgaggttggtcgtttgtacagaggattcttgttagaagcttactctagtcatgattgtattagaatcttcatttaaaggctcctgaagggtgttggcattagtcagaactgtctcccagaattttatttgtcttgtgatagaataaagcatagttagcctaaagagcagttttcctaatagctcggcatgcccaaagattctaggagttatacaggttgaacatctaatccaaaaatctgaaatgctccaagatacaaaattttttgagcaccaatatgatgccacaagtggaaaattctgatgtgacctcatatgatgagtcacagtcaaaacacagtcaaaactttgtttcatgtacaaaattattaaaaaatattgtataatactacctccaagctatgtgtagaaggtgtatgtgaaacataagtgaattttgtgtttggacttgggacccatccctaagatatctcattatgtatatgcaaatattccaaaaatattttttaaaaaaatccaaattctaaaacacggctggttccaagcgtttcgtaagggatactcaacctgtatagcaaaatgaacatatttacatattctctaggaaatattagtttacaatttttctaggcaaattataattgataaatcataaagaaaatttaaaataacactggtaattttcctacctccttcgttattgttacag

SEQ ID NO: 101 - RNA, 331 nt, Homo sapiens
  source 1..331: mol_type = other RNA
gtgagtttaaatatcattataaaactaattatgtgtaaaatcctttagtgacctggaaattatatagctttatcatagttgataatatgagaaatggtctagtttaaatgatcatttattatctatgatttacttactttttattttctttaaaatctgttttaaatatattgtaacaattatagatggattttcctgtgatctcgttgtaaattagcttatgacaaatatagggtgttacaattattgtaatttggtttggtaatgagtatgcaattgaaaagccaaacactgaatggtatatttcatgattctatattaaattccacag

SEQ ID NO: 102 - RNA, 2648 nt, Homo sapiens
  source 1..2648: mol_type = other RNA
gtaagctattatgtggaaatgtgccacccattgtaatgaaaaactggttgacccctagaaattgaaataataaatgtgtgttgtcttaagcttgggttatgttttcttttcccatgtgaattgagatattcctggttcttcatatgccacataattttggtgtatttttgatcttttgaatattatattgtgagactctggttcttgtttaaattctatgggaaaatgtagatacttttgttttagcatgcaatcggtctaattaggttcaggccacaagttccaacctcatttcttgggctgtggttccatttttcaaagccttttcaatactcttcagatctgtcctgcctgtgtacctcacaataggtgatctggtatgtgagctatgtaccattagttcagttcttagaaactttggtattctgattaggatcgatccatacatttgcagctcaagagtgagcccagaagttcataaacaactttatagggtccctttcttgagctcctccctctttgccatctctctgatactttgtttccctagggatttccatttggggctttagttacccagtgatgccatgtacttcaggaattgcacacttctgcagccaagcaagcaagaggagagtagaaagaggaagaaaaaaacgacttttaccttaccctcttagtatcatagctctaccaattggagatttccctcccaaaaaatattagcttctgtgagttcccattgcagcctctattaccactgctatgggatggcttaagggttggggcatgaaagaacagatagaagaaaaaaaaagtgaggtgttttcatattgtctcttgagtgttaaaagattccctttctctttactcgagctagaattagaaggtttacctggagctctctctgtcagtgcagacacccatcttcaggtttcaaataatgttgtcttcagggcaggcagtaacagaataaaagaaaaggtaaattcatcacctgtttgctgctactttaagtcctggtattctattgtaatctgccttctactcctttgcaaagtcctcaaatggttgctccatgcatttaggagagagaagattgaatgtatttactccattgtacctggaaccagatgcccttgccctgcatcaccccatgtcatttcttagcagagcctttgagatttttgtgtgtgtgtgctttacaatctctttccaagttatatcttctgatacagtcatggtcgtgaaaagcaaaataaaatcatgtgttaacatttaaaactttttaattttattctgacaacagctaaaactatttaatcttctgtttcgctcatttcttccaaggtaaacttcagttggttttacgtgatttgctatttcttcttctttgcatttacaaatgatctgtgatcatattactgatctttgtaaagggctaatatctacctgcaacatttggatatgacagtatttaccctttgtaaatacacattttctatttatcttcaaaaattaccattcattagtctgtgttaatgtctgtttactattgtgtcattatgaatgtgatgtgaacatacgaagttgaacttatttaaacgaacactctcatgagcttctaatccacattccttccttttccttctaagttaccatttcttaaaaatcttttagaagtttccttgatagggaaaacacaaattattgaggaatttttctttctcttgacatctgtttatagttactctcttgttccagcagtggatatttcccctccatgtttttctttgtctaaacatatgttcaaaacaaaacacttttattcttctttgcaggttttacaaggatcaacttttagttttgaaacctgctattacttttagaggccattttttttttctctaataatgtgagttcatgcgggctgaagtaattggaatactttatagaaaagattgaatttgtcttctctctgaactctagtttgaatttctaaattttatgaatcatctagatattaaagaggaggggcatatcaaagaggagaaccctagcagagataagaggcaagagtaaatgtttcatgtatgggtaagagtggatttgtatttacctaagtaaaggtagaccctggacaataaggttggatagatgtggaggtggcaaaccatggagggtcttgtaggtcaagtggatgtttttagacttgaagtgttaaattattatctgaaatcattaagagtctttttagatccttgagcttcttgagaagaccatggatattatgcagttattatataatgttttaaaatagtaagtattttagtttaactgtcttatgtaattccatataaatggatgcatgttctttaaaaatgttaatgtatttcagtaaatcaaaatatactttttgactcatcatttaaaggaggccttcagtgaatgctctgtagaggattattttataatactaattttgatatcctaatttatttgttataaagtttagaaggtttgaagaatttaaaatatagtgttaataaacacactgaacttttctttttttatcttgtatttttatatagtacaacagaaaaaagatgaaatgtgaatagtaaagagtctgtgattgttgttcatag

SEQ ID NO: 103 - RNA, 4406 nt, Homo sapiens
  source 1..4406: mol_type = other RNA
gtaagtgtatatcttttattatttttttcttttttccatgttaaaatgcatgaaagtgaaatcaacttctttcttaatctggccaaaagcattacatctttctcattaatagtaatacagtaaattcaacttttatttttaacaggtagtgatgtgtaataatttatttaatcctttttaacataataacagtaaacttaagattcttaagcttttcataaagctcataaatgatttctagaaattttaaatatgtagttatcattatgtattttgctgtagcagcagtatacagttaaataaaataggaaaacatgttccaagactgttttcattcaaatatttatgctatatttttagcttataaaaactcattaatcattaatgtaaaattatttgttggattttttaaatatttagtgtattatttttgtttcttttttctttccatgtttcttcattcttccaccttaagcagaatcaggtgtgtgacacaactatgttttctatccttgttaccattattaataaatacaagggcatgatatttttcacaaaagaaacactttgttcagaaccaaaaaagatcatggcaacagtcagaattaaaaatggtaaaagactaggtgccaaagatgacttacataattgggtacctagaaatattctatggtattacagtaatgatgaaaaatacaaattagaacacattttagatcctattgagttaaataaatcagagtcaagaccaaacaataaataaagtcaatttacgtcaacaaatggtaagttggcagattttaactccctttttgaaaatgaaccatgatcctaaggttggtaaaattaatcaagaatgttgtcaaaatgataaagataaaaatgaggaagagaataagataggcaagagtgagaaaggaaagagacacatagctgaaaatgtgagtcacaacaactacatagatccgtagaatctgctatggaggactgtgattatgtgacagttgctgatgccgtggcttagtgagctgagggtgatgcacaggcaggcgatgtaactgatgcgtcagtccagccaagaaaggacgcgtccctggtttggctacgtggccgtcctttatttctttgttaactgaattttcttatagtaagtagcttacgtacatatatagtgcaaatgggaaagtgtgtaagatttagaaaaagcattaactattagtaaactttatcttaagctctaacttttgattagttcctacaaaaattagtgaatatgcattttctaatttagtgctttttttttttttacaattggtgttcacttaatgttatattagataaatgaatagcaaaaataaggtactttagagttgattgttttgccttacaaacttctaatccatccagctgtatttagaagtaagatctcactacagcgaattatatcagtaaaattttgttacagtgttgtgcagtgtcctaagatgtatactaagttccttcagtggctttttttgccatgttttataacagataattttgttataatgagaaaaggaaacttggatgtgttgctgtctatattgtgttaggctcaggcaggatgctgtggcttactcatttaatcactttgggaggcaggggcaggaagattgcttgaggccaagagtttgagatcagcttgggcagcatagccagaccctgtctctacaaaaaatttagacagatgtggtggaacacatttgtagtcctagctattagggaggctgtggtgggaggatcatttgagcccaggagtttgatgttacattgccctattgcactccagactgggcaacagagtgagacctgtctctaaaataataataatgataatgataaatggtgttaggctctgtgcctaagtatatttttcacataggctgggtaaagtggctcatgcctgcaatcccagcactttgggaggccaaggcagcaggagcatttgaggccaggagtcaaagaccagccttgagagaccccatctctaccagaaaaaaaaaaaaaaagaaacaattagctgggtgtgattgtgcacacctgtagtcctagctactcgggaggcagaggtgggcagatcacttgagcccaggagtttgaggttatagtgagctaagattgtgccactgcactccagactgggcaacagagcaagactgtctcaaacaaaaacaaacaaacaaaaagcactttgcagaatatcagtctaactctacagtttatggactttttatgtacgtactacttttggctagcttacattgagatacagaataaaagtttgttcatagcatttatcgtttttttctttatactgtccacctgagatattccagtcacctaagtcatggaaacatcaactaaaattaaatatctatgttaagagaaaatggctgaaagtgatttaattcataacactttttttcacatgctaataaataagagtttgagacttccactaggcattatctctaactcctatccactaagaatttgattttaagtagttgatggcttttaaccggattattcttctgtaagagtttggaagtctcgtgaagttcgttatacaagaattctgtttacaagagagcattacattagaatttgtttttcagaaatttggactatctcaacgaatacctttagttttattatttcaaaatgcaagggaaaaaatgagccataatcactaatagtaactgcatcatattttagtgagaaatgtgttaaaaatatcctcatgtgagatcttccttagatagaattaccctctactctaatatttaatatattttatatctaccaatcagtgatattaataggtgtttatcatttgctgaatcaaataggtacaacagaagacaggaagtttgggagatagaagagctcagggacaggaaatcacagatgtccatatctgaaataaccttaaaagttatcctgtctaatgccttcacttataaactgtagtggtagaatttgcctagtattaacctaatagtggtagatttgaatgtatacttgggctttcttattaagtggaaatgtattcctgtgatttacatatatcaacaaaaatgtttgtcttcttttttttgctacgacatatgtgcatgtgcacacacatctcctcaaacaaaaatcagatggacacatgcagtcattggatctaaaagatgttataaagttgtgtataataggtattttataataatatattttaagacccataatgtcggtggagtaactgactttacagcccatcaagccaatagagagagaaaggagaaaaaaatgaaagttgtgctgaataattaaaaaaaattatttcctatgatgcttataacagtcctatgaggtaggtggtattctaatttatagaaaaaatgcatagaaaaatataattaagcacagttaaaaaaaataaagtttagaatgagaagtaacaacataaataatgacccaatgtagattcaggtcaaaagaaatgaaaatataatattaatggttttcaaagagggaaccattactttagctcaaagaatgaaggagggctttccgaaggagtaaagaattatggcagttcttttgtagcctagtgtattcatttgctaaggtggctgtaacagactactacagatttggtggcttaaacaatagaaatttatggtcttagttctggagacctagaagtccaaaatcaagacatcagcagggttgatttcctctgcacaatcagagggaaagatctttcccaatcctctctccttggcttataaatgtccatgttttccctgtttctttttatcatcttccttctgtacatgtctctgtgtctaaatccccaaattttctcttttcataaggataccagtcacagtcgaatagggtttaccctgaaatctcattttaacttgaatacctctgtaaagacccagtctccaaataaagtcacattctgaggtactggaaattatgactttaatatataaatgtggagggtaaggggaacacagttcaacccataacggttagataacaatcgtgctttattttggactagtaaaaccaccatagatcagtttaaccattatgaaattatacatgaaggcattatatgtatggacattattaagtcatacttgctttgcttccattgtaattaaaacaaaccatactacctttgttctgcaagttttgtattctaacttatttatttttggctttcaccagaacactccgattttctcatattcctttgaggaaaaaaagttaccttttgacagtattttcttatccagtatgtcttttatggcttttatttattaaactttaaaaatattcctaatttcatttccctgaag

SEQ ID NO: 104 - RNA, 1202 nt, Homo sapiens
  source 1..1202: mol_type = other RNA
gtgaatttaatgttttttattaggaaatctaatgcctaaaactccttccttagttgttatgtttacttttattagcttattaagaagtcaaaaatgcatattcctaatatatcatggtgatggtatactttatacatttgctctttagcatttatttgttgaaggcctactttatattaaacactcctccagatgctgggaaacagcagtcaaaaaattccttatactcataggacttacgttctagtggagaagactgacaataaacaagtcactaaatagtatgtcatctgatgttagtgctaaggagagaaataaagcatgattggtgtaaagagtatggggagagagaaggggtgtaactgaaaatagagtagtaagggaggtcttccttaataagatgatatatgaacagagagctaaggaggggtaaaggaagtgagtcatacagatactagaaaaataattacagacaacagaaatagcaagttcagatgtcctaaggtgggaggatgcgtggtatatttcattaaaaattatcacactgtaaaatataagaataatttgtttcttttagaaattttactttattctgatattaataatgattttttaatctttggttttccaagtcttaccctatttatgggaatcttttttttcttttggctagctaattgcttcagttttgttttctaatctagaatgttagcaatctgttaattccactggtaatgatatagttaagctatgtcttgcttctcacactttatttatttatttactcagggcactaatctgccattttttcgcactttttttccttttttttttttttggtactgcttcttattctggtttttacattgatagaaccaatgttagacgttcatttgccttttgctgtgtatatttgggtaaggatctatatgtgcaatatatgggacagttaaaatcagaattctaaatttgtattattgcatcaggcaataatgtgggaaataccttgacatttcatatacacaatattcttgtattaatttaacgtcttagttcaaaatcttccttgttaatatagagaccctattatttggtttggcaatacagttgaagagattgatggttcttatgaattgtttgccttttcttttcaatggctgtagctatgttaaattattacatgtttgcttgttatctttcag

SEQ ID NO: 105 - RNA, 1697 nt, Homo sapiens
  source 1..1697: mol_type = other RNA
gtatgtgagaatttaccatacatttgttttggtttcagcagtgataagccagaaatgaaaagtttagatatgttgtaaaagtactgatatgcctctacaagtgccctgtagtttcagtgtttattctgcatctgtaatataaaacagtaagcatttctatgtgtctcaaagtattttatcatctgttataccttacatactttcatctctctttttattgaatatgcctccataccttgaaaacatttaacttccaggaatccttttgtttatggaggtaactgctaactggtccttggtccaatgctgccattttgtaaccatttgttatgatatcttcccagcttggtataatgttttataattacattgttcctccccctctttttttgtgttcttgtaattttctccctatgttattttgtattcattttatataatgaataaatgttgcttatgaggtcaaggccaaagacttaagctcctgttgatttcatgttgctgagtgtcataaatggaagcaatcataatgcagagtcattctggtagtaatattaaatatatgatggattcagtgaaaatattatgtgttattagaaaaatattcagaacaggccgggggcagtggctcacacctgtaatcccagcaatttgggaggccgaggcgggcagatcactggaagtcaggagttcaagaccagcctggccgacatggtgaaaccccatctctactaaaaatatgaaaattagctgggcatggtggctcatgcctgtaatcctagctactcaggaggttgaggcaggagaattgcttgaacctggcaggcggaggttacagtgagccatggtcacacaactgtactccagcctgggcgacagagcgagactccatcttttaaaacaaaaaaaaaaaaggaaaaatattcagaacagtatcttgctggcagcaacatttgtttcatcaatgaaaatatgtgttaatttgaccttttctatctaagttaattatgaaagtgcatactaaaatgatgtaaaagtttatatttcaggattattcttattcatggatgattaactaaaatgcaaaaagaaattaagcatactgtttggctaaactgttaaaaattatttttattttaaatgataagcagttaaacttattaagtgatgactcatctctgctgatatatttatgcaaggttttttatttcagataactcttctatttatattaaacagaaactgtatttctaagcaatagcatttcttagagaaaattgcctctattatgttgcaattaaaatttaattactcatgagctctttaaagacacaatttctcttgtgtggttttatttcatataaagaaaaactctgatatactggagagaacattagctaaatagactatttagacttaatcattttgatcagacatcaaggctagactatttaagctgttacttattagctgcatgattttaggaatgtcaaatttcctaagtcttggttttcttgtatttaaaatggaaattataattcctatctcatagaattgttttaaggatgaattgaattaatacagttttgacttcaaatattaggaattattgagtataataagcctgttgtattgttggtacttcgtattatacttactaaaatatttgattaaagatttaacatattctttcgtag

SEQ ID NO: 106 - RNA, 809 nt, Homo sapiens
  source 1..809: mol_type = other RNA
gtaattttttttaatgtgatcatttttaggggaatattttacgttttgttactatttaggaaaatttcaaatatgctcattactatataaaatggctttaatgaatacaatacatattttataaatatagaaaaaaacttatgagaggcaaggctaagggttatagagtaggtctacctgatctttcttgttatttcaagaccaatacttttcacttttctctctgacagcatagattaattacctgtgtctctcttttttttttcttttgagatggagtactgctttgtcacccaggctggaatgcagtggtgcaatcttgactcactgcaagctctgcctcccgggttcatgccattctcctgcctcagcctcccccagtagctgggactacaggtgcccaccaccacgcctggctaacttttcgtatttttagtagagatggggtttcaccatgttaaccaggactgtctcgatctcctgacctcgtgatccgcccactgcggcctctgtgtctctttgtgaaaatacagatgcccaagctcccatccctgaaattgatttaattattttagggtgggtcctgacacagatatgtatgttgttgttattttaagtcatcaatttattctaatatgtagccaacgttgggaacttcgttctcactaatattcaaatgaagactttaattctaatcatatcaaatatggtttctaaaactactttgaagatttatgagtttataagattatcttttatttccttgttttgataatgtatactttttattttgtttgtttttttactag

SEQ ID NO: 107 - RNA, 877 nt, Homo sapiens
  source 1..877: mol_type = other RNA
gtatgacttttatgactgattataacttttgatttttattttacttaatacctcttggaaaaactggaagtagatccttgatgagagtgtctgtaaaggtagatattaagagattgaggaattgtgtttctatgcctgctgtcatcacattccaccatgaaaaacattgataataaaagttaatacatttaggctgggcacggtggctcacgcctgtaatcccagcactttgggaggccaaggcgggtggatcacgaggtcaggagatcgagaccatcctggctaacacggtgaaaccccgtctctactaaaaatacaaaaaattagccgggcgtggtggcgggcgcctgtagtcccagctactcgggaagctgaggcaggagaatcgcttgaacccgggaggcagaggttgcagtgagccgagatcgcaccactacactccagcctgggcaacagagcgagactccatctcaaacaaacaaaaaaaagaaatgatctacgttgcttacacataccttatgcttatagctaggtctcgtaagcattaggaagtcaaaacaaagaatcttttacatgtgtaaaggtataaactatcccatttttctaaaaatatagaggaacaaagtgtcaaatttaaagtaatcactagtaactaaatatattcctctgacctcattttcgtgatctgttgttctaattattattggccatattgctgctttaaaggagagatgttgaatttgttgaaattttaatcagcatttagagccccaggttatttttgttttccaatttgtaatgataattttgaatacactgaatctatgagaacagtattatgttttctcataaaatactaattagcatttaatgatag

SEQ ID NO: 108 - RNA, 3130 nt, Homo sapiens
  source 1..3130: mol_type = other RNA
gtaggaacagagttttaaacttgtacaaagtttaatcatttcaaattttggcattgttttaaaagacaacactattctggataacctggtttcttcctgatgaacagtttgtttggttgttgttttaacataatactttttttctgttgtagtattgttggagactttttcttccttgaaatgtttaacttgtttaaccttgtttgggtggcagggcatggaacagtgtagagctggggctgggcgaaggagttggagctgtgtgtgcgtcatgaagctgtcatcagctatgagcctgggctgaggctgctcagcttctcctgggtgctatttttctccaactgcagcttcagcttcttgattgtataatttgcttcctcaagtatgagccaggaataattgagctgtcttgtcacaatgtgtggcatactggatctaggctgtgctgcaatgcttttagagttatatcctgggcaactttctcttcagatagccccaagagatgaattcagcaccagctttgatgttttactagcttctgctttctggtacttgattttctcccaccccgaacacatgggattccaacctgtgaaactaatttttgtggctatgaaagaggtagtggtagtttatgagtaaacattcagtctgttgccactatcatcatgtgtggttcatcatgactgtgatgagtaggtaaaaggctctttgtgtcattctcatttccaattttaagcagctgcttcaaggagtctggaagtcattgaccagtgggatcctgcctgtgtcttttcccattaaagccatcctgtatgaagtggtatcctttaccatctagcacatctgccgcccccatttcaaaaggcatactcatctttatctcaacattctcatacagttccttatgtccatgcacctccaatgtcccctttgatgtctttgaggttttcatcttccatgtctgctatttggaatggtcttgatgggaggcaagatagtgatcactacaactaggatgggagtcttagtaccgtgaggctacagcaagtcccacagagggcctgctgcactgtacttgcctctgtcaaccaagtctaaggagaaagattaagcaggcatattaaaggacagcccagatggacatgaagtcctggaggaggccttggttcctgtcctaatactaaacctagagtacccagaatccacacttctccactctagctctcacttttcccatctacacactgggaaaaattattctgtcagaaagccagtgtcaaggtgagaacaaataacaaatgtgatgatatggagtgggagaaggggtctcttctactgtcttattggaccctagcagtggctctgagccagcagtcctgtcagttgatttcttggtcgttcctttgttttcttctataatcacatgtggactcagaatgaattttgagttactctgaaatctatttattcaacagatatttacttagtacctcctattgccagactctgctttatgttggatattattttttaaaagcccaccttgcctagatttcctcaaaggaccaggtggcttccctggttttgaaagaccctaattcttactatgatcttaagtaaattatatcctttctgtgggctcaagttctttctaagagggctctttggggctacaaaagaaattgttagtgcaaaaagagtttataaggtttataaatggttagtagaggtgatgatgatatttaaccataattgaagatgactttgcattttagatcatatacgtgtttttcgtctgagaacgatacaggtcactgagcataccataagccttcagtaaatcatttgcagaagacattgcagaagacataagtctaagtagaaatctcttgacagagagaaggctcgttttgatccttgacctcaaatttaggttccctaaatccattaaaaaagagaaagaaaaagaaaaaaagttactaaagtttaaatctgggaggattatatacccttctcaataaagcagtttagagagatctcttttgggacccatgacacaggtcttgctcatgctgacatctttatagttgctttattatttattcaacaaacttagtaacacgtattctatgtcaggccttttcctgactactgggacaaaccagggtgatgtgggggctgttttagatagggtgatcagaggaggcctctctgtttgggtggcttttgaatagaaaattagatgaagtgaaggagtaagcttctgatatttcactgtttacttgtggtagatctgtgataatctctgtcaggttaaaaacattcccttctaatctaagtttctaagatctatcaaaagctgtttgaatatatttagacaatcataattttcctttcttgtattatcctagcagattttgttgccaaagctatactggccattttaacttagaatgcagtctttctattcatttctctggaaaagtttggatattgtaagcattatttttctttaggtatgatgaacctgcagaactgtttggttcaattatgaattttttttttctggagtctgtatttttttgaactattaatcatttctttaatgattataaatctattcagatttttacaagctttatccctctcccatcatacactatttttcttacccatgcttttgcacaattttttcctctcccttagtgttttcctacctagatacctcctatgtgtgtctgtgtatgtgagaaaagctttttatttgccatctttatatttctaagaatatctagtaatacagaattttatattctgaagaattttactttgcattttcttattttgtgattgaaaaaaggtattaattttaaaatggtcaaatcaggctccatccttggaaaatacccaaatcctttattttgattgggccatctgttaattagggataccttatctcttgccaccactttttaatgctaaataaatatgtagctaaaactttgactagaagaaacagtaaaataagatattcttgcttatttttagtacagttatttgaactgacttttaaatcagtgacataaattatttgccatgtctatactttttttccttatacttttag

SEQ ID NO: 109 - RNA, 1162 nt, Homo sapiens
  source 1..1162: mol_type = other RNA
gtaagtaacgtaatttttctttacatgataaaataatgcataatatcgcaagatgttccttgcattgtcttatatagataaaaatggactctattaagaagacccatctaactgaagggcaccccattcacccatttgcttaagccagaaactttggatcatcaacgacttcattcttttcattctccacattttctatcattaaatcatgtcagctctattttcaaactatatcctaaatatgaccacttcttggtatcttgagacatcactaccagtcttgtccaagctattgttttatacctgaataactgcaataatttccaagctggtatctcagcttccactcttggattatttcaccctatttctatttctgggctgtctccacacagttgccaggtaacccttttaaaacataaagcacatcacaaagcacaaagtcctatcctcagaatcttccagtggttctccatcaccctaaaataaaacttaaaagttcttttcatatcccaaaacaacatatgaggtctggcacccagttttcttcccaatctcatcttctactacttttcccttcatttcattcacaatgttttaaccacagtaaccttctttcagtactttaaacaatccaaactcgtttaagcgtcaagtccttatacttgtttcctttgtttagaatactgttcacccaaatattctcatagcttgctcccagacttcatgtctctgctgaaatagaggctccttagagagaccttccctaaccctaaccctaaccctatactacttgccatcactctttatcctcttaccctggattattttttcttgatagctcttcctaccatctggcactatattacatcatatcatattaaacacacattctttgtgcttccccactaaacaaggaccatgcaagatggaacattgccattttgttcactgctgttagcctctgtgcctaggacaatgccagttatgcagtagttactcaatacttgttgaatgaatggtgaatagaacatagaaatttgcctatgcgtgcttttgaaaaccatattttaatattacgctttgttaaaaatgtgtatctttataaatcctcatatttccatggcaaaccttatcttctaacttttcattgtcctcaaag

SEQ ID NO: 110 - RNA, 593 nt, Homo sapiens
  source 1..593: mol_type = other RNA
gtacatcatgtattcatatgactactttgtttttttctttaaaaaaaaaattattagtttttatatactccgaattgctacaactagagacaagcatttttcgactttactgcctaacaggcttattaggtccttatttcttccctctaatgctaatcactctttttcataatacacactagaaaaaaaggataaacccaactctaagtttccagtttgtaatttagtttaaacttttctaagagcatagaatgagttaaaccttagcttcccagaggaaaatactaatgaaagagaacaagtaatttttttactttcaggggtctctgtagcctgctttcattaagctcctcttataacgaaaccacacttgcaaatgccatcaggtcagatattaagaaaaacgtgaaggcttttgtattccaggctttttgtttgagaatggtgacattgtagcattgagagtaaatgtttacttcgataaaggctagcttgttctgattactgtacatcactagttcataagaaatgcccatatattttatgaagcaatatctgctttatttttttaacacattatcattgtgttctag

SEQ ID NO: 111 - RNA, 3218 nt, Homo sapiens
  source 1..3218: mol_type = other RNA
gtaatgtattttaaaaaacatgttagctacccccaagtttttgaatttgggtttgccttttttttttttttttggctcagatttctgatcattgtctccctgtaaaatcgaattcctgataagctttgggtcttttgtctctctgtgctattaatataaaaatattcccatttttctctttgtgttgtttatactatagagtagcaagtacccaagtgttcttctctttgttctccatctgggtgttacagatttaatcacaatacagtgctaagcaatgaatactaaatctgttgcttccagtttctaagtataggctctttcaagtcctctgaacatttttaaaaactgcaaataagtaaatactgcctatatttttttccgtttacaaagtaaaaagaaaatctttctgctcccttccattcccattcaaaagtgattactaatcattcctcattcctgcatatacatacacacatattttgtatacatatatatcacacatatgcatacatgtgtttgtatgttcatatgtacaatgtacatatcctcattatttgtggattctgtattttctaaatcacctcctcactaaagtgtgtatgtaatcccaaatcaacactcgcagcacatttgcaaacatccacagagccttggaaagtttgaataatccaacctacatgtccccagcagaagtccaacaaggcagtgctcagtatcctcatttcagttttcatagagaaatgagcagaggatggagacagtagagggcagcacagcatagtgcaagaagctgtggctctggggcctggtggaagggatttgaatcccaattctgaggcttgttactgctctagccttaggagagtcatgtaacacttctgaatcttgttttcttatgtaaataaatagaatttaccaggatgagttatctttaggatttaagattatcatctgtgtgagatatgtaggtgtatgtatatatatgcgtgtatgtatatatatgcgtgtatgtatatatatgcatgtctgtacatatttcccgtagcagcagtggtttgatattcactaattgggctaactttatagaccaaaactactatggatagagaatactttgtttgcatttacgtatatatattttcttggcaagtaacataaaattgaactaatactatacacatttctagcatatttgcctttaacagtttatcatggacatcttttgaggtctgttcataaattatctcatccatttaataattccatagtgtattattgcatgtataagcacatcgaaccatttatgttttgatggatatttagtttgcttccaagtttctgcttctataaaatatgattaatctattgacctaattatgccattgtgataggatgatagagatgccattctctccaaaggattataccaatttatatctgaactatctttgactatctcttgtagctttttcagtatgctatgtagtcctattactaatttgtaataaaagccatcatgtgtgagttgtactagacactatgctaattgccttacaagcattctatatttacaaccatatatgataggtattactgtctccattttatgtgataaacaaattcaaagtggttaagtaaccattccctaagccagctaggaaatagaggcaggattaaaatctaaatgtatgaaactccacagctccttggcattcctagtccttaacccgctatgctatgctacgtcttggtaactaaaagtacatattaaatactctcaaaatatgtctcatagcagccagcttggtatgtacactagacacagtattaatgctgttgatgtgaggaaaattttataattttccttccatccatatactaaccaggcccaacagtgcttagcttctgagatcagagatcaggtgcatgtgcattaagggtcatatggccatagatagttctctaatctttccattcctcagtttcttaagggaatttctgaaccctcaaaattccttatttcctaagtagacagattacctgtcatttttcaaagattaaggcttaagatcaaaccagaactgttttggaaattctaaatcactgtctatataaatggcaagataacttttaagatatttataccaagcccagtacagtagcacaccacacctgtaatcccagcactttgggaggctgaagtgggtggatcacatgaggtcaggagttcgagaccactctggccaacatggtgaaaccctgtctctactaaaaatataaaaattagccaggcatggtggcacttgcctgttatcccagctacaagggaggctaaggcaggagaatcgctttaacctgggaggcagtggttgttgcagtgagccaagattgcaccactgcactctagcctgggcgacagagtgagactgtctcaaaaaaaaaaaaaaaaaaaaagatacttgtcccagccatgaaaatgtttgctgccccttactttcgcaaacttttagtattttattatttttcaatggctgtaaaatatgacttattaaatgtagtataatataaagaaaagagatatctagcaaagatagcattaaagcaaaaatcctatttgcctgctgataaagttagaggtgttaacttggagggtgaatccaataaattagaacttttgtgctatatttggagacttttgttttcctaccaaagtatcagggctatgtcttacttatctttgtattacacagcctgcatgacacgttttgcacatagtaattgcacagtaaatgtgtaataacctacatggaatagccagtgttgtgttggatagcgggagcatttggctagcttatggttatagtcccttacccaacagtctgcttttcttctgttgtacttttagtacctaacaagtttccctggctttaggattttttccatgtaaaatttctatcatgtgaagaaaaaataacttggcctacacttctaatacctagcacatacctctttctgcctgctatgaaattataatacttgatggagggaggcagcattaagtgtttacatcctgaagtatttcagccataacatccagtgttttccaggttctaggtttcataaaatgtatctctgttctctagaacaaatccattaccttgaactcattcgtagtgggaaaaagctgagtctaatttgtatgactttttcaacag

SEQ ID NO: 112 - RNA, 939 nt, Homo sapiens
  source 1..939: mol_type = other RNA
gtaatatttaattatattttagtatcgttttgtgaaaacagctgttgaaaactattttcattaccatctttaactacgtatcctaaaaaattcagtaataacatcttatatttgacctttatattgcaaagttaattatgttcatctgactattcctaacatattagagttaacaaaaaattcagactcaacataggattaagtagtaaatttattttttaattgtaacaaatatatgccattagtatgttcttaagttttgggtcacattggcaacagtgtctttattttttttttgaaattcttttcaggaatcctaaggttatagttcccttaaaaaaatatttgctgttttacctcttttaagactgtaaacaggacaaaaaggcatggatatgagaattagctagtgatcactggctattctaaatagtcactaaggcttgaattgtctcttcaccagatgcctgtcagaagtcccaaaggtttccctgatcatattaataactttataaaaaattgatcattattcattaaatattagatattagtaaggaaaatataaatgaagtctaaaccaaaactcttaaccagactaacttcaatgttatgaatcacaaaatctttttgattgattgctctattgacaagctcttatatgcttttagagaaagattaagtcccattataagagatgataaattttagtcaaagactagaacacaacttacagaatacataactggacttgacagttaacaacttagttatttacactgtacaatggaacaaagaaaaatcttaattcttctgcctttattgctgtatttgaccattcaggaatactttggctttcatatttacaattaaatctccttgttcaaacgtaaaatatgtatatttcctatatgcaacttttaaagataatgtttccattag

SEQ ID NO: 113 - RNA, 100204 nt, Homo sapiens
  source 1..100204: mol_type = other RNA
atacttgggaacaacttatgtatctaatttttagagtacttgcaatgtttaagagaatctaacatcctggagacttgatacattagctgttaaattattgttaaattaaataatttaaaataaattaaataatttaaaaatatataaaaatatacaataatatgcataataaacaatattaaattatgcatctgattttttagagtaactgcaacctttaagagaatctgacatcctggagactttgatatattagctgcataactataattttaaaatatacaataatacgtataataaacatattaaacataataaacatattaaactactgactatgtagtttctggattcacaactatagttgaaaagtataataattagctaaatacacaaatagtattggaatttttataagaacttgggatttgctatagtttcaagttttatttcctaaatttaatcagttatttaagtactgagaaagttttctttgttaagttaggcatctgaagtacttaaaaatgaatacattcaaaataattcaattcttcaatatatctttattttacatatatatacatatatatgtatatatatatatacacacacacacacacacacaaatatataggtatgtatatggttcttctgttagttgtgcctggggtcactcatgtgcttatagttatccagtggcttgattagggctggatggcttttaaagggtagatttctcagctttgaaaactagtctctgctacttattaacacatcctctaataggttatcttaggcttcttaaatgatgatctcaggacaacaggagggcaaggttcaatgcacaagctctttataaggctctatttagtgcgtattttaaaaattattattcaagcaaggtacatcctgaatcaatggggaagaagttataaaagggccaagataacagaatttttaagattccccaagggagcatagtatgagagaaagggagagagagcatgttacgagctcagttgtggagtcatcttaggttacttccaagagccacctgcaaagtagaggttgcagggaagggacagatttacacaaaggcataggcagacggggacctgactcactgggggccattattgcaaacatgtaacacactattcttgttagttgtgagaacttctaatttttcctaggcacagaagcagacttacagatgcagtttaagtggatgggatgactgatttctgtttgcttacaaaaatggggtgttactatatatgtgcacaaaatccaagtgcaattggaaatgtgaaaggaaaataaaacttgggatctcaattcattatgtctataagaaaaaattagggtgaaagctgagtcatgcaagaaactgctctccttttgttcttagcagacagctacacatcaaatgttaaatatctccacaggtagctactttaggacattcatcttatctcacgtaaagtgctgatttaccaccccacgtgaatacataatatctatttccctctctgctctttttctcttgcaacacatagattaccatatctaccctttttctcttccaatggatttttcccctttaaatactgaagccctcaaaatcatcttagagaaaggaagagactacagactgtttctgtgattcagtgtttatttcttttcggcatgtccctaaccttggcaaaataaacttctaaattgattgagacccgtcttagacactttttggttcacaaagacttctcatgagctacattatagatatacaaaactaaatacaatcccttcaataaaactgtaaattgtattaaggcaaattgttttgccttaatttcatggacaagaatgagtgtgcagcaagcagatacttgtcttccttcaacatgaggaaactggaaagatcagtagggagtcagtgtgaattaagccattaaccatggcttaatttgccatgaagataaggcaaaacaaaatgtttaggacacacaatggatacaaaatggccatgctttgttttagagatcggagaagtaagctactatgacttttcttggtaagtcagcaaaaaggaactagaagtaacatccaaggaatataaattcattgctacagagaacaaaggtaagcaataataaaaggtgaggccttcttgtgaagtgattttggtcagataaaactgttcacaaaagcctagggcagaaagaaggcaggtccagacaagtgttcttataacaaggccacttacaacattgtcacataagtcccatttcttgatgaaaactgacaggtgcagaaaatctgacgaaaaataatagttttgccaattttcctcaaaaatatacagtgagttattgaactagaacctgagttaaatttcctgactcacacttaggtacagacgacaatgcataaaaacttagttgacttgttcaagccagagctgacttgttcactgctgtatccccagtgcctagcacaggaaacaggataaaaaatactgttagaatgagttaaagaataaaatttaggtaagattttgcttttagttacaattgtcactgagatctaggttacaatcttagcttcatcaattattacttaacttctatgaacttgttctcacttctgaaaaattagaataaaatttaccattagagaccaggcaccttgagtttatgaatgtgagcaagagggtggtggcagaagggtagaaaagagtaatacagacaaacgaaacttgtaagtgaatacataaaaataagttataacgaatgaaaaaaaaaacagaagaaaaggtgttagagaaaacagacttctattatgaaaacaaacacgcagtagtcagcatagtgtatgacataacatgtgctcagtaactattaaatttcattcctaccattgtcagaggtatttgaatcacaacaactccatcttgaataggggctgggtaaaataagggtgagacctactgggctgcattctcaggatgtctgtcagtcattctaagtcacaggatgagataggaggtgggcacaagatactgatcataaagactttgctgataaaacagcatgcagtaaagaagccagccaaatcccaccaaatccaagatggcgagaaaggtgggacgtctgctcgttctcactgctcattatacactaattataatgcattagcatcctaaaagacactcccaccagtgctatgacagtttacagatgccatggcaatgtgaagaagttactctatatagtctaaaaaggggaggaacactcagatctaggaatggcctaccctcttcccagaaaactcatgaataacccaccccttgtttagcatataatcaagaaataactataacaatccttagtctaccacctccaatgggggtgtatgcacctccaattctttcttggcgagatccaggagccctctcctggggtccagatcagaacacctttccagaaacaccatcccatgttaaattatgaattaagaacactgaacatgaagaatgtttaattcaaattattgagtaaacacttacaaaattaaaatataacatttaataaaaatagcaagattgaggaacaagcacttcacaaatattggtttcccagaatgtctgaggaggtgtatagaagttaagaatatgtcctccagccagagagatctgtgttcaatcacactatttaagttacttagcttctccaagcctcagtttcttaactcagatggaaaatgaggacaatgtaagcacctatttcactggattgtaaggaagaataaacaagataatgaatagtatttggcatataatctttcagaaaagtagtaaaattgtcattcctataaatctaactaatgtctttaaaattcatctcacaaaatcagtatctttttattttgtactgagctacttattttacccaaattgggggtttaaaaagaaactcattacttattttcgaaaggaaaatttactgtcaagtacagtaattttttttttaaagcagcgtttaaaaaaaaaaaaagacaaagcactggtccagatagttggaaatgtcatttagatttaagtctacagcttcttaggaaagttaggtagagatgtatcatactccagcatgattgtgagcagacgagcaggatgagcaaaacttgcacgagtaagagtggtaaacaccagagaaggcgctttcatggaagggcaccctaggtaaccaagcaagtggcaacaatagtacaggtgagtccttggatcccttctctctccacccccggcagtggtccggacaagtactaacctctcctctctagagccttgactttatactagctccacaaccatcaagccccacgatccgcgaaaggaatggtgctacaggtaaggcgggcggggtcgggatgccgagacctcttgtcaattcttcgctcgcccccaaacgaggatcacggctgcgcggctctcactctaactatcaagacagattctgatactaaacaaggctaaaactttttgttttttccttcttacagaaggcactaccctggacgccgcttgcaaacgcctgaagagaaggcagggtgttcccactttttcagaatgggaccaggccctacggccccaatagcagaagcttctctaacccaggactgctcccctcaattcctcccaatatgctgaccgacagctcgacctcacctgggggcggttccgccaggggatggcttaggaaaacctcaggaagagcagcccagcgagtgacgccactgaaaccacagtcatggtctacctcgttcaggccaccagtttgcaggcgcctgagccgctgctcctcccgcctctcggtccttccccgccgccccggtaccgtcgcagtcttacgcgttgcatgccgggagcgccttacctcctcttctcagggacttcagttcccagcatgcagaagcctcaggcagaactgcactctgggatttgaagtcctcgttccacgccttctcatcatcctgaacaccgagctctgggactccggcggagaatctaaacgtaaagcatcacccacggtcgtgaactgtaggctctcctggcatccgggatcttattctggccttggcggagttggggatggtgtcgcctagcagccgctgccgctttggcttgctcgggaccatttggctggacccagagtccgcgtggaaccgcgatagggatctgtcagggcccgcggccgggtccagcttggtggttgcggtagtgagaggcctccgctggttgccaggcttggtctaggtgggtggatccttgtaagcaggattagcgagtcactccacgctcaggttctttagcctgagggcccgtgtgccacagcatagctaccccgcccttccagcctcgggtccctaatactgccttgcttcggttccagtttccgccgcacaacttcactcattccaaatgttaatttctgcgttttttttcagccccaattctgtttctccaaatcagggatgattgtcggccttccacagaccctcgcgcttgccaggattagggtgttcgcgcgcattgtgggtaggggtgtggaggaagggatccagaaatcttaagtattaacttagattagtgttagcaaggaagccgtcacattttatttagccgggacactctgacagtttgtgccgactgctatttttgatcaaggctattttgcccacttgtctattttgtggcccaattgtctgttttgctaacatcagaaagttataatgaaataatctgcaaaaaatgtaaggtgctagaaaaccaataatactgtgtaccttgaaaatgctaatatacacctgttttgttacagaggtggagcacagtgaaagaattcaagatgccacctaatataaactggaaagaaataatgaaagttgacccagatgacctgccccgtcaagaagaactggcagataatttattgatttccttatccaaggtgcttaattggtcaataataatagatatatacattaacttatgattaatttattaataaaatatgaatttatttttttcagggacaactataattgtcacaatctggaagtgttcttatattttgcttgaaggttataaaatataaaacagttgcttttctgtttacttaggtggaagtaaatgagctaaaaagtgaaaagcaagaaaatgtgatacaccttttcagaattactcagtcactaatgaaggtttgtatgtagtaggttttaactataggtttggctattagtggaactataaaaatctgttcttatataaggtaatctttgtgaaaatacctggtaatatctacatcaccactaaaaaatgcaatatatttaaatgtgaattaagtattttagtgtataaaacattgctagtttctacttaaagtttctaaaagggtgtgtaggggaaatagaatgagtatgttgaaaagtaacataaggaaatatatcttgaggtccaaatgacaaatgcagacaatgactgctatagggatttgttaagaggggaaatgatttaagagatgtcagaagacttcacaaaggatcaatactgaggagtagtgttagataagtggaaggcaatgcagtggtaagatagtaagggaattctagagctgttggttaccataaataaatactgagaacaggaaatatgtttattctttatatttgaggaaacaaggtgcagcaagtttgtagcagactgtagagaaaacaaatcttgggtaagtactttgagataggttgttgagggccttaaaggtgtattttatgctatcagcaattgagaaggcagtaaaggttttcgaaacacaattgataggtacaaaaatacaccttaagaaggcaaaactgagtatattatgtaggacaaactgaaggaaattggagctttgtagacatcacattatagcggagtttaaacctgaaattatggattagaataatagcaattggaacagaaaaaaagtagtggaaagacattacaaagggagatgttgcattactggatataagacttgaggacttgaggtaaaaaggagaatcaaaaatgtttcatgctattaaaaatctagaaattgtagtcttaagtaagaaaattgcctggcatggtggctcacgtctgtaatcccagcactttgggaggccaaggcaggaggattgcttgagcctgggagttcaagactagcctggataatatagtgagtccttgcctgtacgaaaaaatttgccgagcatgatggcacaccaagcatgatggcacgccaagcatgatggcatgcacctgtagtcccagctactcaggagactgagatgggaagattgcttgagcccaggaggcaggaggttgcagtgagctgagattgtgccactgcactccagcctgggtgacaaagtgaggccctatctcaaaagcaaaaaaaacaaaaacaaaaaccaaaaactatttattcagcaaatatttactgaacgtctccatgtgccagccattgctggcactaaggatcataacaaataaaacagaatttttattttcagtgcttacattccagtataaaggcatattgaaataacctttttttaatgtttagatgaaagctcaagaagtggagctggctttggaagaagtagaaaaagctggagaagaacaagcaaaatttggtaagcaccttggaaaaagtttattatggtattaaataatgaattccatttgttcattaaactgtagaaaattaaattatattctataaaatatatatattcagtttatttttaatatataacatttaataataaatatttctagactcctattttatggatctgccatataatactttttgttaccttataatcatgatggactcttttaaaagaattaattttgttattgaaatttatttaaaagtttgttttgtggtaactaatcaattaaaacgtttttctttttttttaaaaaaatagaaaatcaattaaaaactaaagtaatgaaactggaaaatgaactggaggtatgtctttttgtattccctaggatgtaattgtcattaattttattttgaattgttttcaaattttaaaattattgttggctggaaaaattataaggatgattgtaatcatggttatttgtttattctgtatatgttctacatgcctattatgtgccttatatagtactaaggactgagcatatggttgtgaacaaaataagaagttaactgctggatggagcttatagtcttgggaaatatacagaaagattactagtaactgaggtggagggtgggtggggatttgaggaatagtgacgaaagggtgttatagaagtaatttttgacaaagctgaaggctaaaatatgaatgtattgttgaagaacaaaatacattgagattcctgagaaggtaggaatgtgatacaaatggatcagcctttgaaaggaggaatacccttttcctttgtgttaggagaggaggatgagtggatgagcgtgggaagagtggatgtgtatagaggcttttatgtttgtaggcataatgcttggaagttgaggggttggtgatgacatcttctgttaaaaagagtgggaaatggtgtggtcacattttaaggaaattaggtaaaatttgaaatatattggagacaggactggagagttggggatctggagtcagacagatttgagttctagtcctgattcttctactcgttaactctctgaacttggatgacctattgtttttgattgtatatccagctcctgggaaaatgccaagcactttcaataaatactaaatgaattatggagttggatcagttctgtgttagtgtttagctaggtagctgctgtagaatagaagggtagcacagttgaagatattggtaggaaagtggttgaagtgatgattatgaagtcttaactgaatagataaaatcaagattggggttgggtgggcagaagggtagggatatggagggagaagatgaggggttagagtgtcctgtgaggtcgaaggacaggcatagtgggaataattgaaagaatgttctggttggacaaggatctgatgtgggtgtgggagtgagagactatagtgaattcaagaaaaaaatagactagaacaaaagttatgtggagattgcttagtgggcatttgatagacatctgtgggccacatgcttaaattcccagtgcattttgcggagttactggaaggttggtggcttgtttctaccatgagtaggtaaagatggagagcaggatattttgtgagaaagcagctgaagtttctataggatgatggaggaatgataggaatgatcacctgaagttgcagggtggggtaaacctagaagcaccaacaccttcttctgaccctcatgtatttggaatctgaaagaatgagcaccttccaattgaaagagttccaagggcattagtatactaaaggatccaaattgcagctaagccaaggagatggaaaggaggattcagtaaagaatctgaggatgtgaaatattaatttatcttggaagagaattttagagagcacaatggaatgctttttggaggagagaaagagtaagaacaatttggttaaggtagaggaataacagaactataaggtgaagaaatgaatgtgagacacattagatgaccaaatgatttgatgttcttggccatgacctgaattaacaagactgtgaggtaaaatggatttaatcggctacaaatcttaagataaccaaaacctgagctgtttaatatggtagcactagcactaaccacttgtagctatttatatttacattggttaaaattaaaatgaaaaatttagttcttcagttgcactagccacacttcaaatgcccgaacatagctacatgtagcgagtggctattgaactggacagcactgacagcatgtccattatgctagaaagtcctatgggacagcactggtctaaacagtgcatggtatgagagaaagggcaggttaaggcactcagcttcactgactggggtggagattctgatggtttgtactcaggttccagatccctgaggctcaggaacctttgcagtttagtctggttacctgtggcccagtggttacaacagaatgattaacagtcaattctttgcatctctgggtggctcaggaaaaatttaaggagttattagctgtgaactaaccttaagtaagttaaattaaaaaaaaaaaagttcttaagctaatatgattttaaatatctgcactgaagtataatgcaaatttaaattcagcataattatttgcttgttgttgactcatttgaacctcaaaatataatgggattaatttatactttgggtttattactttaagatggctcagcagtctgcaggtggacgagatactcggtttttacgtaatgaaatttgccaacttgaaaaacaattagaacaaaaagatagagaattggaggacatggaaaaggagttggagaaagagaagaaagttaatgagcaagtaaagcactttttttttccatgaatcttcactgttcaagttacctggctttttattattattggtaacaatatcaatttttatattgtatgttatatttgaaaaatgatgtacacttatctctaaggttttatatcactgttcattttgtcatcaccaattttaaaatataatggtacttctagtgaatatgacttgaagattaattctttatatttggaagtacatttttctcaggacatcaaacttgttacctaaaattaatgcttttgtctggaagattggtatcaagtaactaatagattttcataaagaagtgatctttctagtgccatagtttattttgggtaaaagttatatttgttcatttcaatgtatttatatgattagtagattcgcaaatgaatctttcgatatattcaataatggttaattaaatatcttgtttttggttgtaccttattttatgtgagatatatatatatatgtatagtttttgaaaagttgtgttcatgtcagcagtttataaatcacatatttaaaataacatttttaatgcatagtttttattacctcgttattccttgttataaactaataattcttgcagtgttcacttgaatttagttttaggaaaaaagttttttgcagatcaacttgtatttcctggaagaaaatttcctattttacctcagcttcctatttaatgtattatttatttatttacttaacatttatttgttttttatttcacctgaactgttagtaaacttagtaaaatttggtgcctacatgtggtaactgtcctgtcccttatactcagaaacgttttccacctttgtgtcctttaggtcattgttgtgttatattccatttattttattttgtccattgttctctcagaaattgagggtcatacattttaagaaaacaatgatatgctatttaagagaatgtatcataaattgatttgtaaggaaaagtatccccattcttcatgtatgtattttactctaaaatgttgaagaatcatatagaagttagctatgaaaacaatgtggtagagaaagtatggatcgatgccacttaaatgttaggaagaagctcttagagcattatctgtttagctaactgcaaaacatagcagacatgtggattttttaatagtcatcaaggatctaacttataatatacactggtagaattgcttagggggatgtctgtggttttctggacttttgttcttctatatagacctgtatcagttgacttatcattcataccacacacccttagctaatcagaactaccttgtccatttatatcttagactattgtcttttttcatagtcacacacagagaaaacttgaatatatggcctgtgttcctttttggctgctcaattccttgagatgaaatatgggtatgggttgctttggcaattacttctttgccgttaaccagtcattcagttttattgagtctttacagcataccagaggctgctagttactagtgatatagtgggcaactatgttctggttctcaagaatattcatagtcaataataagcataacatagtgataatatgatacttagggagatacataaggtcatattctggcatactctggagagagataccgtaatcagccttgaggtgcaggatgtgatctgtaaactgagacctgaagtatagttagactggtaagaggaatgaggatatatatggtggttaataaaagaacattctgggtagaagatatagcatttgctaagacctagaggtaagagatgttatggagtatttaggaaactacagttattcattttgactgaaatataagtgaaaatagctttcatagagtccttactatgtgccaggcacttcatatgcattaattcattattgcttatttgatacttgtcatatgagatagttgtcatttctgccatgatacagatgaagaaatggagacacagaaagagtaattgcccatggttgcacagcttataaatggtaaaggtaggatttgaaaacagtcttactcaagagtctgtgctatcttgccttcccagttttattttttatgatcctctggagagataagcaagggccagttcctaatgaatttggttcttttcctgaaaggagccagtgaagagttttgagcacaggatatcatgatcagatctatactttaaaagtttactgtactttgtagagagtggattgaaaagggccaagactagtaaggaaacatttgtgttaattcagggaagtgctaatgatggcatttgcctgagaaagacaagtgtgagagaagtagatgtaattggatgtggtgaatgtaattggttgttggaggagagggaggatggagagtctgcctaattttgtgggttgggccactaaataggtagatagtgccattcattaaggaggaacacaagaggaatttggaaagcttgagattatttcagttttgtagatgttgagtttgaggttcttctgggcatattcaaaaagggtatctgtggatatggaattcacaagagaccctgtacagatgatgaggatttatgaatcatcaatgtagacattattgaagccagagaagtgattgtaaggcacgtctctgagaaatgtctaataaagcaatgaaataggaagagtgcttcaaggaaaagctcaagaaaggagaaacagagtgtgatgtttgagaagacaagggaaaaaaacattaatagcattaaatgctttagcattaagttcttggcttctcttcttgtaaaaatttcccaattcagaacacagtgggattattaactttcaattgataataataatgataggcaaacttctaaaatttgtattgtagtttgcattttattataaactttctttaaatttttattttgaaaaatgtcatatcttcataaagattgtaagaaacacactgttggtgttaatgtaaattagttcaaccattgtgggagacagtgtggcaattcctcgaagatctagaagcagaaataccacttgacccagcaatcccattactgggtatatacccaaaagaatataaatcattttcttataaagatacttgcacacatatgttcattgcagcactattcacaatagcaaagacatggaatcaacccaaatgctcatcaatgatagactggataatgaaaatgtggaacatatacatcatagaatactatgcagccatcaaaagagaatgagaggtcaagcgtggtgactcatgcctacagtcccagcactttgggaggccgaggcaggcagatcacttgaggtcaggagttcaagaccagcctggccagtatggtgaaaccccatctctacaaaaacaaaacaaaacaaacaaaaattaactggtcatggtactgtatgcctgcagtcccagctacttgggaggctgaggcaggagaatgacttgaacccagaaggcagaggttgcagtgagctgagatcgcaccactggactctagccttagcaacaaaactagagtttgtctcaaaaaaaaaaaaaaaaaaaaaccggaacaagatcatgtcctttgcagggacatgggatggaggtggaagccattatcctcagcaaactcacacaggaacagaaaaccaaacactgcatgttctcacttataagtgggagctgaacaatgagaacacatggacacatggtggggaacaacacacactgggacccgtcaaggggtcggggtgggagaacatcaggaagaatagctaatggatgctgggcttaatatctaggttatgggttgatctgtgcagcaagccaccattgtacacatttacctaagtaacaaacctgcacatcttacacatgtaccccagaacttaaaagttgatgggaaaaagaaaaacaataaccacccacatacccttcatatagattcaccagttcttaatgttgtgccaactttgctttatctttttgtcagtatttttacacacacatgtatttctctgtctcttgtttgttcaatcacattttttgctgagtcatttaagagctaattgcagatatgatactttgcacttaaatatttcagcttgtctgtttgaaaaagaaagatgttctcctacaatgaacacaatataattgtcatgctcaggaattttaatattgattcaacaccattatctagtccataatgagatttcttctaatggcccaataatatccttcagtctccccacctccaatatccaaagttctgtcaaggatcacatactacatttggttctttattatagactttttaaatatcgttgtataccattgtgattctatcgtctcctttaataaagaggagaaccagaaaaatgaaaggtcataagaggaatgaggtttggagaataggtgaaaaaaggcatcataatgtttataataatgtttgcctgttcagagaaacaagaatcacagataaagtcacttatatgtagataagagaatgctgtattactttttgctattctattcactgatcatttttctaagaactctgtatgcttcttgtttaactcttatgtcagcatgtatgagaaaactgagttaaagagatgttaagtaactcattcatgctttactagaaattggttgatgagggacataaacctaggccggtgtgattttagattgcttcttttaaccattgtgttgtattgccttatatttctaagtaatttatgttcactgagagcaaataatagtctagctatgacttagaaaagtaaaataaagatgttgggcagaaaaccattttattaggggtttttttggaggagcagattaatttgtttctgtattctttggttagtttgtgtgtgtgttctttttaattctttaaaatgaaactgtttaatccttaaatccttaagttttgaaaattttggcctattatttatgtgttaggttgatattaaatccttaatagctttaacattttctactttgttagagaggatttaaaatttaagtagataagctgaatatctggctttatattaaattactgctgatggccaggcacagtggctcatgtctgaaatcctagcactttgggaggttgaggcagatggatcacttgaggccaggagttcaagaccagcctggctaacacagtgaaaccccgtctctactaaaaatacaaaaattagccagttatggtaatgcatgccagtaattccagctactcggtaggctgaggtgggagaattgcttgaaccgggaggcagaggttgcagtgagccgagatcgcaccactgtactccagcctaggcgacaaagactttgtctcaaaaaaaaaaaaaattactgctgaattttatcttcttcttatttattttttttttttactattttagttggctcttcgaaatgaggaggcagaaaatgaaaacagcaaattaagaagagaggtaaaaaattttagtagttgtggtggttcaacaaaggtacttattaaaataagtacctaagtttacataaatttatattttaaccaggactggagtcttctaagtaactgatgttttcagactgattttatggtatgactttgtctcagggaaatagaaaacaaagcaaaatgtgaggccattaagtattacattcatctcaggtctatgcgggtaaatctttttttgttgttttataagccattctttgctagttttctaattgaatagatgactggatttctattcttatttctcttacccagaatcctttaaaattttttgttacttgtggaatcttataaattctgattatcatttggttctactgagccaaataatgtttgtacattgtttattctgatagaagttcttaagtttctaacataattgaaatattatttgttttggtagataattagtattctttctttggttattcaagataatatgcatcattttcccaaaatttttttgttttctttagtttctgattattatttttaattatgtattacctttctcatttctaattaccgttttcctgtccttttctgtagaacaaacgtctaaagaaaaaggtgaggctttaagtgtggtgaaatcttgggaatttaaaatatgttgtgagagcactatttagaggatatgattttgttattctgaatagttttgtaattgaatgttgtgtttggttaccttcagaatgaacaactttgtcaggatattattgactaccagaaacaaatagattcacagaaagaaacacttttatcaagaagaggggaagacagtgactaccgatcacagttgtctaaaaaaaactatgagcttatccaatatcttgatgaaattcaggtaaaatggctagaagtcaattcagagcaatggttcctaaaaactttaatttcattacaatgtaaatataatatttagccctacatgtaaattccctggtataaatctgtcactatgtacttgtaaaatgtgaaataaattacatctttgaagttgcaactttttagccatttttatatttgcctgtcttggtcattaagaacaattgaggtccttatgtactattttcttgattcaatttgatttaattggtcaatgccaattagtaaaggtctataaagaattctctttttttctagaggacacttatggctgcgtttaattttaatttggtttaaatttcagtttttttaaaattactttttaattatagtgtctttaacttttttagactttaacagaagctaatgagaaaattgaagttcagaatcaagaaatgagaaaaaatttagaagagtctgtacaggaaatggagaagatgactgatgaatataatagaatgaaagctattgtgcatcagacagataatgtaatagatcagttaaaaaaagaaaacgatcattatcaacttcaagtaagaattacttttagaataacttatttattcagacttcatattatctcattactatttatttgacactagaaagtactttttctaggatgtgaatttttgtctgtctttttaatagtgtaatatcttgtcatgttggtatatttgtccatatgtgtttctccaatcacctcacaaacactaatttttgcaatttaggatatataaatgatacttgaatgaatgtgtagatagcagtcattatggggttttctataaaagactactgaaaatcctgtggatcataacatttcattttatcttaaaataaatacattataaatgtattagaaaccaatacattgttcagtatttatgtggattaaatttgtttaaaaggtagaataatgtttaaaaataaaattttctagtaatgaaagataattatgcaattataagatgcagaaactattaaatgtcacctataattccaggatgacttcaatgataaatacacatatgtaatgtaatgtatccgtatgtatgtgtatataagtatgaatacgtatgtgtgtgtatgtagatatatttatatatataatgtatatgtaaatatgcacaggtgtaaatatatgttacatcagtttgcaacaactcttgaaataactttgtcttttaggtgcaggagcttacagatcttctgaaatcaaaaaatgaagaagatgatccaattatggtagctgtcaatgcaaaagtagaagaatggaaggtattttttttcaattgacataataactttttctttttgtattttagatttaaattttagtcttatttttctttaaatgtcttatactggtttataacacgtttattagggtttttaaacataagtttattttatttattggttagaaaagctctagaactgtcctttttgatctctagctaatttgttattgaatgacctctttcacatcaatgagtttaactttaaactttttgatagaagtctaactccaaaatatatttggcatctaaaatatataattcgaaatataatttaaatttttttacttaactcatagttaccttatatacattagttaaatagttgcaggtttaattttagtttttctaactaaatgtcaggttcatcagtgggaatgggaataagcaaagggatcagaataacttgggaagccttttcaaaatacacttttcttcctcaccaccactctccaaccttaaccaaattgtcaggccttaccatattagaagctgggattatgatggttgtatacttgaaaaacatcagagattattctgaatgaataattctaattttaaaaactatcacttctagagtcattgctttctagtatggttcacataaatcttgtgggcagtttggaactggttagcatctagggagctcagataacctatattttaaacaaaagcattagcaatggaaataaggcctatagaatcagtcatgtctccataaactttatataaagggccagacagtgaatattttagaccacctggtctctgctataactaaactctgcttatagcatgaaagcagccattgacaatacgtaaatgagtgagcaaggtggttttccggtaaaattttatttacaaaagcagatgggaggccagatttgacctttgggccatagtctaccaacccctggaaaaaacagttgtctttaccagattgaatgttggcagggtaaatggtgacatgttatatgtattctgtactttgttttgacttaataccatttcataattattttatatcagtacgtatagtattgctgttctttttaaaggctatgtaatttttctttttatacaggtgttaatttgataatttgtgaagtttatgaagtttccaattttggggttgtaaactgttttaatgaatatccttatatatgttattttgcaaatgtacaagtatatctgtggaataaattgctgcaagtgttgtaattgtcatgtatgttgcaaatacattctaacagtttgtcactttttttgctttatggcattttttgctgtgaaatatttctttttatgcttagttaaatttattattttttaatgacttttgacatttgttataatgagaaaggcttctgagtataaacttgttttctcatcttttctcctaatatcttgttttgtttttgtttttgtttttgtttttgagacagagtctcactcagttgcttaggctggagtgcaatggtacaatctcagctcactgcaaatgccacctcctgggttcaggtggttcttgtgcctcagcctcctgagtagctgggattacaggcatgtgccgccatgcgcagctaatttttgtagttttagtagacatggggtcacactgtgttggccaggctggtcttgaacccctggcctcaagtgatcctcctgcctgggcctcccaaagtgctggaattacaggtgtgactctgcctggcctttttttacatttaaatcttcgaaacatataattcattttgatgtaaggagtatcatgtggattcaacagagctactctgttgtccaaacatcttttattgattatttcatcttttattgaattgattgatctattttctagcagtgtatacttgttttaatttgtgtatgttttaatatctaaaaacgttattatttttctgcttttagacttctttatgaatatttttaatgtgaattatagaactggcttgtccagttcttaaaaaatatcttgtggatttttattgggtatgtgttaaagttataaattgttttatagattgatttaggataaacctttttatgttatttggtccttctagctaaagaacacaagataccttttctttcattcattcaagatattttatgcctcttggttgcattttaatgcatacttcataaagatcaattgtataaaacttttcacagttgtatggaagtacttcttgtttataaatgagttttgaaaggttgaaatatttttaaagattgaattataaaaaaagaaaattcggtatatattttaaaatcattttctatttgaatttcaggttgtatatacaaaaggaacagagattatgccagtagttgctcatactttctcatttcaaataatttttattttctgtatcataaatctactaacggtgtttattatttatgataatgaagaatgttttattaactttccttttgcataacagattctattgtgtttatttctagctaattttgtcttctaaagatgatgaaattattgagtatcagcaaatgttacataacctaagggagaaacttaagaatgctcagcttgatgctgataaaagtaatgttatggctctacagcaggtaaaatcttaacagaattttgtttatcaaccagttttattacagttggaactctgaacgatgtcttttatttattatatcatcagtgcctagtgtagcggctggtactaccaagtgtataataatgtcttttgaaatttcttctaccacctggtcccaataaaaaattagaattaagtttagatcacggattagacttagaactagagttactgtgtttatttttctatgtttatgtggatagtacacacattgttttggttagaaattatttaacaagaaatgattaaaaacttttagaaatttaaaataattttatactcttttaaggtttattttactgtatcttagtcctaacataccctatacaatgtgaaataagctaaaagcatggttataatttgactgtgctacctattttatttttagtgaaaataacccaaataaaaggaagtaatacttttattatttgtgctgtagttatagtccacaagtaagaagatgatttgaaaagtgtatgctgaataagaacaattacaggggacaacattttttaataaagtacgaaaggggaaaaagctaagttgaataaaagagaaagcacagagcaaaacagaaacatacaaaatggtaaaaaggtggaattgaatggaggatgaggaaagtaacatataaggaagtatagaagccataaacattagggagttctggaaatcctattttccagagtgttagccattatatccatctttcagtattggagtaacagcagtgtacctatcattgtgtattacagttgaagtgtacaaaatggtaaaaggcatacttgtacccacaagaaaatatgttctacagtcttgttgaaaaaaatcagacgtacttttttccttacctttttaggttaatattcatgaagggatatatattgttttaaaatattttatagggtatacaggaacgagacagtcaaattaagatgctcaccgaacaagtagaacaatatacaaaagaaatggaaaagaatacttgtattattgaagatttgaaaaatgagctccaaagaaacaaaggtatttttataaatatatagttattttatatacaattatgtttttaacgactttatttttattaaaataaaatgtcaagtcaatattgagttttctccatttgaattttatattttcaaaaaattgtacaagatatttattattatacttatattactagtgcttacatttgtaaatgatggatgcattttctattatttttctcctctggtgaaaattacattaacgtttattaccaggtcactggtatgaaagaaatgaaaaattgtgatacaattatttttatttaactttttataattaacaaagaatggaagataataaaattttgaccagtgtaacagcattgcagatagttttcagaggtaatttcacattaatcttacccaaattaatgtttcatcatattctccttaccctgagccatattaccttttttaacacatcaaattctatgaatataagttcttacaatatctgtgttgttatatttccatagcactacatactatagttatgccagggcacactagtgcgaactgttcatgggaaattcatggacatgtttattataattggtgactatgtatatatgtatacactacatttatacacacgcgcatggaatcactatttcttcttcatgtcatatatatatacatatatacacatatatatacatgtcatatgtgtgtgtgtatatatatatatttgtatatatgacatgaagaagaaatagtgattccgtgcacatatgtgtgtgtaagtgtagtgatgtgtttgcaggtacggttgtaatttcaaaaatgaagcaaaagccttgctcaggagataattgaaccaatacttaaaggaagtaaaggagtgaaacatgcagatggctctaagcagtgggaataagttcaaaggcagtaaagcaggagtgtaccaatcatgtctgagaacaacaaagaagtctttttggctggagtagagtcagcaagtgaggcagtgataagaccagagaggtaaacagaggccatatcatatggggccttatagttcattgtgcagacttggcttttaagtgagaagggacaccggggaaagtttctgaagatagaaatgatataatttgacttaggctgtgtttgcagtagactgtaggagtggtaaataagaatcagggagacctgttagaagactattgcaataatctggagaaaagtgatggtggtttggggcatggtggtagcagtggagttactggatgcagcagttctggatgtattttgaaagtgataaaaatggaatttgctaacagatcagatgtaggatgtgagagagagagaactcttggtctgaaccaaaagttttggtcatggtggggttgtgggaagagcaggttgagagataatcaggtacttaattttagacatgttaggtttgagatgcttattagacattcaagtgaaggtgttaagtaggcacttgtatataaaagtttaaggtttaggacaacaatctaggctaaagatatgtttggtaactgtctctgtaaaagtaattgaaataatgaggctggctaagatcaccaagggagtaaatgtaggttaagaagaaaaatctaaagagcttctactttagcagctggggagataaaaaggagctaccaaaggagactgaaaaggaaagcccagagagctaggaggaaaagcaggagtatggagagccctgaaaaccacatgaggaatgtaaccaaggaagaagaaacaactgctttcagagctgtgttcattgctgctgataggtcaagatgatcactaaaagttgactattggacttagcaatggtcatttttggttcaagagaaaatgggtagagaggaaatgtaataaagaaatataggaacccttttccaggactgtttctataaagagaaggagaaaacaaggtggtagcttgaggggaaagagggattaagaaaacatttttctctttaagatggaagaaataactcatgattttaggttaataggagagctccattaaagaagaaacattaatgaatcaatgaagtggagagagagaacttctggaacaataatatttttaagaatgcaatgggatgggatcctagtgtgccagtgaagaggttggccttaactaggaacacagagttcatccataattgtagaaaagaaggtagagtgtatagatatcgatgtaggtggcttggtagacatcctggtaatgggaatttgtggaagttctaaactggttgctgcttttttctcagtgaacaagggagcaaggttcttagctgaaggtgaggataggagaagatgtttcataagtttgaggagaaagaagagaagtgaaagtataaaatggtcatctgaaagattgaagacgtggagaatgtggtatgactgttgagtaacttcaagagcccacgatatatatatgtatttctatttatgtgtttattatatttgtatcagaacactttgaaagtagtttaaactgctttaaaaggatgactaatagtatggattgtgcgtattctaattactaggagaaaaagtggcaattgatctctgctgtcaaataaggaaaaggacttatctgataacactttagtcagtccgtagttatataatccctaaagctcacagaaggtgtgtgtactagactgtactctacatcttgaacttaacttgtaaaacgtaatggctaatggtattcttccttcataagattaggattaggtttagttatcaggaacagagagctgaagaataatggcaaaatcaagatagacatttatttctcatctatgtaatggcctagaattaagcattccagggtgttgccttcatctgccccatccaaaatggatggaatgcagctttatctcatgtctgtgtcccaaacagcaagacagaggaagaggggcaagagttaaaagcatgtgctgaaggataggcaggtaaatatagtgtttattgtgtagggccatgtggaagaatgataggagaatagatatgtggatggaagggagaatagatactgggggacaactcagcctgtgtcatgttccacagcttagatgttagctccagacagctgtgctcatttcttaaaaacttttgtgatctcaaacgtactagttttatgcctaagtccaatattaaatatataacctatatattagtaaatgcttataatgaatgagtgtgagaatgatctgtcaatcaattttggaatgatagcaatattatgttttggtcttttaacaatttagtaagatattacaagtaggcatttaggaagtttttagcttagtttggattaaatttagctgcaagtgacagaaaaatcaagcataatacaataatttaaacaagatagaaatttatttctctataatatagacaaagttgaagcaactagggcaggatttgtgtgacagatgctcaaatatcccctatcaggaaccctgtctcttgttgctgtgcctatctcaacatgtggtttctaactcatgtgaagttgccaccctcatatccatgtggatttcagctagcaggaaggaggaaagagaagagagattactcctttattttaaaaacatttttttttttttttttgaaattcacatatgaactttgcgtttatattccattactgacatgaccacacatagctgcttgtgtgtaagtggaaatttagttctttatttcaaatggccacgtgtcaagctaaaaatccatagttttagtacagtggacaaaagggaggttaaatattaggaacagctagcagtctgtatcacaatgatcattttttgtaaagcagtattttgcaaccttttaaaatccataccccttcagctaagaaggttttactgaacttcagttttttagtaaattgtattagtaaaaccaaaacaaaactttcatcttacaaatataaaatgacaactttaaaggatttttttttaatggcataccacttttcttgccaccatgttgggatcactgatttgaaggaataagtagtcaattcaattcatgatttttgtttttactctgtaggtgcttcaaccctttctcaacagactcatatgaaaattcagtcaacgttagacattttaaaagagaaaactaaagaggctgagagaacagctgaactggctgaggctgatgctagggaaaaggataaagaattagttgaggctctgaagaggttaaaagattatgaatcggtatgtatttttatcttgtcattcaaggagcttagaattattcttgccattcacagactattctgtgctatttactgcataccatttaaaaaacattccataagtatcttttgataaagattatcctcattaatttatactaaactattgaaacctttgagcatttactttttgccagaattgttttcaaacttttgatcacagtgatttgtccaaataatcagttttggtgaagcagcaggattacttttttttattatctgtgttcattgggccaccatgtagatgtgacaccactggccaatttgacagaatttatgacaggaacatactgtgtcaatacaacctgctctccactttttatactttttcattggttacaactaattcaagcaactaatgacttacttattctactggtattgctgatttgcttttactaattcttttagtattttggtaagtgttttttatatgtaatgcatattcagagtcactttgcctttaggatattatactggaaagttttaactgttgcatattacatcattattattactggatttggtttataaaagcacaataaaaaaccagtgtaatgatataaattataggcatatgtacattttcctttagacttagtaaaaaaaaaatcatgaacttgataaatttattcaagtaaaccatgttatattttaaattaaattggatatttttcagggagtatatggtttagaagatgctgtcgttgaaataaagaattgtaaaaaccaaattaaaataagagatcgagagattgaaatattaacaaaggaaatcaataaacttgaattgaagatcagtgatttccttgatgaaaatgaggcacttagagagcgtgtgggtaagccatgttttaagttacatagtttgcgcaacctgatttacaagtctttttttttaatttaaattttgtttattattatttattaagtagtttaatgcttttttcaaatgcttttataaaacatttaatacaaataaaagtggagctaacctgattgaagtggaatcagattttatggggttggagtggtgggtgggcagggctggaacattgctttatttggtctagcatctcctcagtaatagctgcttgtttaaaaagatgaaagtttattaataccacatatcagagattaacctttttttttcccaacaaaagtagggtctgtattacccatgtttgtttgcaaaatgctcttgtaacagatgagatatttaaacttcttgctctgtgttgtgattctcctgcctctgcctcctgagtagctgggattacaggtgtgcaccactatgcccggctaatttttgtatttttggtagagatgggatttcaccatgttggctaggctggtctccaactcctgaccttaagtgatccacccgccttggcctcccaaagtgctgggataataggcatgagccaccgcgcctggcctgttaaaatcttttaaagatttttaagtacttgatttttataatttagactacttacgttttactttgttcgagtattttaaggagtaattagtaatatagcttgagagtttatatatttattttaataaatagcctattagttaatattactaatttgagtgttatgatagtgcagactaagttgctgctttaaaatgaaaataaatatctaaatatcaatttcattattgctaaatttcatttaatgctttcttagttaaaaatgatcatttgtaaaaactattatctaaagaaaagacaaatagacaaataagtattttatacagatatatatgtgtgaaaagtatctaacttggatccgtagttgtgctaggaccccaaattagacttctgatcaacttggactatcagatcacagccttctgatcaacttggactatcagatcacagccaagaatctggaagttcctaaagatgacttctggcccgtctaggtagctgtcatagacatcatattttctgtgcttaaaaagctccaaatcttggtttataatttcatttaggtttttgttaggatttccattaataattgtgataaaattttaacttgggttacagtttaaatatctggaaaattctttcacagaaagttacctcattcttcagtgatactggctaagtgaattataaccagttgcttgatggtatatgacatttttgcagcttatttgaatgtttttaagtttttaattatattgctttctattgtaggccttgaaccaaagacaatgattgatttaactgaatttagaaatagcaaacacttaaaacagcagcagtacagagctgaaaaccagattcttttgaaagaggcaagtgtggtagtcagttgattattttcttggctgaactatagagaaatactaataatttatactttgcagattgaaagtctagaggaagaacgacttgatctgaaaaaaaaaattcgtcaaatggctcaagaaagaggaaaaagaagtgcaacttcaggtatactcagttattctaaacctttaaaaagaattattgataagtgagttgtctggatatgaaattatttgtgtcttagctgtttttgctgttctattgtggatctgctacaaatttaataaatgacaataataacctgaaggagataagtgagtgtcagtgggttcagtcctgaatctgaaatagacaaaaacaaaacaaaacaaaataacaaaaaccaagcaaacaaaaaagaaaaaaaccttagaattatggaatttttgaaaagttttatagtatagtattttaatttctagacagcaccaatatgttgttattaataataataaaacttagtagtttttatgttaatatatgttactcaacattttccctttccttaaggactatgcattgaaaagcttttcttgtaagttattattattattattattattaatatttgagatggagtctgtcttgttctattgcccaggctggagtgcactggtgcgatcttgctcattgcaacctccgcctcccgggttctagtgattcttgtgcttcagcctcctgagtagttgagactacaggcgtgagccaccacgcctgacttatttttgtatttttagtagaaacagggtttcaccatgttggcccaggctggtcttgaactcctgacctcaagtgatccatccactttggctccccaaagtgctggaattataggcgtgagccaccatgcctggccttaaattattcttttctaagtgaaagtaatgttttattgaatataaattaacatctttcttgggtttattttacttgagctaaagagaacagttggttaagttttataatagccattgcagtgcttttttgtaagaagaccacacagaaggactgtctttttcacttgccccaaatccccaagcacgtatatgagtaatagcagagtggttctttttagcattatgatttctataatacatccaaaactttctcaagaaaaaacttcatgatttattagtacaataatcagtttactcattactcatcatttatatttactttatatgtcttttaactggtgcttattaagtagcactttaatatagaataggcaaagaatggtagagaagatgaaattcaaaaattaggttctcacattattaatagttcattaaaagtgagctaaatgagaagcttgtattggctatgtagaattttggagggattttggaaacaattattctacctttgcattaaaacttgattgtaggttttaagaattaaagtgttggaatagtaggagggttattttaatgtttttagtttgttaattctcttatatataggattaaccactgaggacctgaacctaactgaaaacatttctcaaggagatagaataagtgaaagaaaattggatttattgagcctcaaaaatatgagtgaagcacaatcaaaggtaatagtaaagtattgcaaagagagtaaaggaaaatatttttttttttttttttttttgagacggagtctcgctctgtctcccaggctggagtgcagtggcgcgatctcggctcactgcaagctccgcctcccgggttcatgccattctcctgcctcagcctcccaagtagctgggactacaggcgcccgccaccacgcccggctaattttttgtatttttagtagagacggggtttcaccgttttagccgggatggtctcgatcttctgacctcgtgatccgcccgcctcggcctcccaaagtgctgggattacaggcgtgagccaccgcgcccggccaggaaaatatttttattgtgttttcatttcttccccctttatctcattcttgaacatctaatcttattattgttgttaaataagtagagggaaatatttgcttatttaacctgttgattcaaagattgattaatgagacattatttactctgaatacagattaggagttcagataaagcagagctgctgcataggagatcatcattcaataccccacagtcagatcagaatgagacagaagagaatatgaccataggatcattatcaagaatgttatctgaaattcaccatagtgtagaaagtggaatgcatccttttgtccctttaactagactttcttcatccatgcaagttaaagagaattcaactccagaaactattacaataagagagatttttaaagcaccatgtctgcagtcttcaagaaatctagaatcgttagtcagcacctttagtagggaaagccatgaagaaataaatgacatatgccttttttctgatgactgtatgaagaaggtgtcaagaagccatcaagcactagagaagactagttttgtacaaaaaagcaattcatcttttcatggcttatcaacagcttcagacataatgcagaagttatcacttaggcaaaaatctgcaatattttgtcaacaaattcatgaaaatagagctgacatggataaatcacaagtagcaacattagaagaagaacaggttcattcccaagtaaagtatgctgatatcaatttgaaagaagatataataaaaagtgaagtacccttacagacagagatattgaaaaataagcttaaggttaatcttccagaccctgtgtctattactgcacaatcaaaattatctcagataaattctcttgaaaatcttatagaacagttacggagagagctagtatttcttagatctcaggtgagtttttctccaaattatatttctgtggttgttcttttatgacgtctctaacaaagttctgtaacaattatagttagaatatttttgtttgcactttaacatcagttatacacattgtactttttaaaatctaaaatgcagtacattgatatgaactcattgacttgtctaatttattaaatttttctttagaatgaaatcatagcacaggaattcttgatcaaagaagcagagtgtagaaatgcagatatagagcttgaacatcacagaagccaggcagaacaggtagtgtaaaggcagaacattaaaagagatgattgtggtactaaagacaaaaaccgttatatctttttgcctcttaccatggatgttgggagagggagaaagtgggattaagatcaccatctgctttactgtttagattttagtttatttttatgattgctgctatgtcttcatagctcgttttttttgttttgttttgttatacttaattgatcaaacttttcttaacttgaaaattatagacttgtgatattttgttgaaaaaaatcaattttattctctctgcttttttcagaatgaatttctttcaagagaactaattgaaaaagaaagagatttagaaaggagtaggacagtgatagccaaatttcagaataaatgtaagttacaattatcttttacttttctgttcttatttttcctatacttaaaatcatgggcctaaaagggcgttaacacattctctgttttctaatctgctttactcctaattacctctgtactgtatatacttcagtctgtcactatccagttgatttgccttgctgttttcattgtgagagaatgttactaatatgaattttttgtgagaatatataactcctttttcttgtgtgttcttcaatcaaaatgaagttagaacaccaaatttaaaatactttaatataaagcatagtttaagttaaggcagaagtatgccttatatacgtgtgtatatgcacgtgatataaataggtctgtcatttaactcaactattcacgttggatttatagttgaatttttttgtatgtttatttacatttggatttttccaatgatgtctttggtatatgtgaaatatttgtcatctgtatagcatagtgtaaattgtgaaaaagatctgatcatccaatgagaaaactgtgtaattacagtaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgtaagttatttttttcatgttaatgtttttcccctatcactttagagagattttctgctgtgtacagatctccatagtttctgatgagatatttttagtcatttgaatcattgtttccctgtatgtaaagtgtagtttttcttgagctgctttcaatacttttcttctaccaattggataattgttattaatctgtcttcaagttcactgacattttcctctttatctgtgttcttttggttcaagggtcagcttgagaccttgaggagttttttacaccgactttggagctcgtttttgctgactcttttcttattgggattttcctttcacttatcccatggctttgggctgtatcctgtggttttctagatgagaaagatgatagatctctgcaattgcaccctgccctatgactaaatctttaaaaatggcaaagtcaatctttgctggtcctgtcttccgtatttgaggggttttttcccaaaatctgcttgcttttgttcattttctagaacatctaggtagttttttttcattcattttttatttatgggagtgtagatctcttaggaacttatgccatcagaagtattatgaaatggctttattctaaatgtttaaagatttactcattgctacaagaaagatttagccatcactaatattctatatatatttaccatatagggacttgagaatttcacaggattcagtatctgtatataaacttgaataatatacacattttagattgttaatatttaagtatatgtcatttatgttatctgaacatatttagcgtacattgtcatattatttcccaaatttgtgcttgatttcaaatgggaaaaaaattcttattatttattgaattgtttttttaaaaaaatcatgattaatcagtaattggatactttttaaaataacactataattgttaacagagaatgagagtgatactggtatgttaaaaacttcctgaggcaagaaaataatttgattcccattatatctttctcatactgactttccttctctgattggtgattttgttttgcctctgccactttgaatgtctaaaatgattctttatgctttttttatgtgaacatcttttgtccgtgatgatgcccactactgatactgtgtcccagatcaaacttaattttccaagggcagctctacttagtgaccaaatgaaaacacagtgaatagcccaagaaatcctaacttctatttatgttgacaatctctggaccttcctgaagccactgtttgcatagacttcatttacttttatccgggattgtcattgttttttcagattcataggccctatctgaaattcacaaatcacctagcaatacttctctaagaaatcttcagaatccatgacaatttagaccagacaatgctggattatgcacttcagttcactttttgttactacaaggtatttttcagtgcccccaacagctatcttaactcattctcattttaccaaagtccatgtagacacggcactattcctcaatgagacaactaactagaccaccttgttgtcagtcagagtaccttcctctacctacttttatcttccttatatcctctttgagttagtataagttattactctgcatgacctgctctaatctccttcaggggaaggcttttacaaatctactacctagagttaaaccccagatcaccttcctgagtaggagattgcatttggttctattcattttaccttatttggcttctaccttcactttttaagacttactttgcctttaacagttttttccatacagttcatctaaagtccaaatatatttattagatgtgtgcattgtgtgtatatacttagatatgccactgttggagatttcgggccagtgatgccactctgataatattttaatatttgacatattatttttgcttactcattattcttagataatatcatgttatgataccttgcctttatttttatttatgcttcaactatgtggagaggaagcactgaaaaattcacttaattgaatgttgtattgatcaattgttcaatattgtattccattcctttgcgcatgctttgaatgcaggtgctatataatttcagagaaaaatacctcattttgactgtacaaaaaccccatgtagggagcagagctcacattgttttccccttttagagacaagaaaactaagatacagagaatttaagtcacttgcccagctgttaagtgactgattaaaatttgaaccctggtcatcttattcccgtctggttgtttttctagtctaccagtctattaagattagctaggtgttttttaattgttttaatgaagtaattactatgcttggtaatgtaaatgaaagttttatagattcataaataagaatttgaattggcatactttattatcatgcttggcaatgaaaataggaaaatgcttaaatgtccattttatttaaagacagactgttttttactatgattttactgtttttctccacatttctaatatataatataaatttgctaggctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaaggtgagaattttattaaataaaagaaaatgctaaacataagaatgtagatttaataggaaatttttaattttttaaaaagaatgctttatgagaaaatgccccttgaattaattctttcaatattaagaaactggatttctcttataaaattataagtggaaaataagtgccttataagattgaaaagaatacaaaaattctaaatctcatacctaggcatttctaagcagaaactgaagtatggttgaggtaaaattcctggcagggcattcacatatctgtcaatttgtctttctttgggtgtaagagttgtgattctcattgctggatttttttttccagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggtattgaaaattttgttacaggtattgaaaattttacatgtgaataacaaaaatcattggtagtatgtttctttatgtttttatttttattttactttattttaatttttccatcaccaaagcatgcagatagtacttttctcaatatttagtcttcatgtattcctgagttctcaaaatagtaacagtgaaatatattttttatggattttgatgttagatggattataaataaaagcaatttataccattcattccattcatctgcatgagcagcatgttcatacatcttgttcgcacacctgtcattcatgtgaaatatatggttcacaagcagaacaacaagcagctattataaagcagtgttaagtaaatgagcacttttatttcttgctgggtggaaaacaaaagaataaagtctgtcaaggctttttagtgtcatgatagaattgttcccctttttgcattcacaagtaaaaactactttttttttgagacagagcctcactctgtcactcaggctggagtgcagttgcgctatcttggctcactgcaacttccacctcctgagtttaagtgattctcatgcctcagcctcctgagtagctgggactacaggcatgcatccctggctaatttttgtatttttttttagtagagatggtgtgtcgtcatattggccaggctggtctcaaactcctggtctcaagtgattcgcctgccttggcctcccaaggtgctagggttacagacgtgagccactgcacacagccataagcaaaaacttctaaaccaaattattcttcatctttgtcttccctttacgcaataaaatgttaatctaccaccaaagaggaaagggtactctactatactacctgccctgggtttctcagttttgctgtctatataatggtcgttatgaatgtcctaatgacagatccttttcattattttatttgaaatttgactatctataacatcacatacattataaatataattacaaatatatgttcagaatcaatgaaaatatatttttgattatatgggccactatttctctctgctaggtgatccatttgtgagtatacttgagttataattattaagtactcatttttattttggaaattacagtaattcatctttttctcaatattgggatttttattattattttatgttgtctaaggacagccttaactacttattagaatattgctttgtatgtgatattattatttttaaatgtataattttaacattattatttctcttatttacctgaggtataggaacactatcagcaaatattggtagtatggcattgtcgtattttttgagataaaattcatgatttttaatctttgtataagaaatatatcagaagtttgtagtagattagagagtaccaactgggagtctgaaaagctgtccaaagtggcaaaacaggtacttagactctcaatcctaaggctgtatagagctataaacgtggcaagacctttggagtcagacagacccaaactcaaatgttggatccatgtatatggaaagcacctgacaacaagcctagcatatgtacttggtaaaaatgattgccaagtgtagtgttaatgagtttttggatattgagtaagttatttaaatttcaatttcatctttaaaatgaaataattggaaaggataatttgagtgagggtatgaaattatgtgttcataagagagggtatgtggccgagtgactagaggcgagtttataactattctatctaataaaactttgtaatctggtaatttgtgtgctaaaaataactttacctgttgtatagtactctttttttatgccttaaactaaagtgttcaaaatatcatggaaaaatgatctgtgttgcttacagatttggtgacttttaactttcctataatgttgtcagaatatgaatttatactttcaaattcagcatttattctattgtgtttttttttgcattcttatttctaaaccacttttcaggaactagaaaataaagaaaaaaagttaaagaatttagaagattctcttgaagattacaacagaaaatttgctgtaattcgtcatcaacaaagtttgttgtataaagaatacctaaggtataggtattagcaaaactataaatataattgcagtatattcttgttaattgtgaaagtaacgtaagaataatttatgttttgttcttcccttcttcttcttcctttgcaattgtatttttttttactctggtaactactgttaggaacttatttatggagacagtgtagcttaatgattacattaagcctgggattatcctgcctgggtttgagtcatttaacgtttgctttttgtaagagcttgagcaagtcatcttacctatctgtgtctcagtttccttatctgtaagttactttgtaagtaatacccttttcataggattattgtaaaacgtaaatgaattattagatgaaaatgctcggactagtgtgtggcacatatgaacagtttgtaaatgttagctgttgttagcatcattcatcatcatcacaatcatcattgttcatatatgtttatagggaactaacatatttctccttatttctgtcatctcatctaaatcaatagaatgatttccttaataggaattagaatacctaatcaaaggtgatttaaacactaagaataattattatctgacctaaccagaaccacaaagctagttgtagggcaggtcatatttgaaggttgttgttatcgcctatgatggttgtaaaatagctgcatgaattcaagaaagatgatgtgcccattgaagaagaggagcatttttttctacatagcttttatttttaaataaacatttttttctggtgatacctggcagacattgactccgatctcatttgctagaattggatcacatgtccaagtctgaaccattcagttgcaaagagaatgataccgctatactgggtttatgccaagaacattacacatgtttgtggaatgctcatgtgtagacaacagtgtcttacacaacttcaaaaaaataatttatatataaatatgttttaaattactttttaaattcacaagaatttatggtatacaacatggtgttctatatatgtatatactatgctatacaacatggtgttctatatatgtatatactatgctatacaacatggtgttctatatatgtatatactgtggaatggctaaatcaagctacttaacatatgtattacctcgcatacttttttttttttttccttgagacagagtcttgctctgtcacccaggctggagtgcagtggcgctatcttggctcactgcaacctctgcctcctgggtccaagttattttcttgcctcagcctcccaagtagctgagattacaggcatgtgccaccacgcctggctaatttttgtatttttggtaaagacggagttttgccatattgtccacgctagtctcaaaattcctagcctcaagcaatctgcccaccttggcctcccaaagtgctgggattacagcatacttcttcttatttttttttttttttgcactaagaacacttaaaatttactctcttagcaattttaaagtatataatatactgttattaactttggtcactattttaattagacttaagatgtgtttgtattcaaattattttgtaagcatttaacacccaaatttgagagtggggtcagaatgttggaatttgatttctagaattagtatagggtattattttcctactttttttctgtgttcaataaaatgtttataagattcagcttcaattatattataacccatttagtggtgaatcagggaagaatgaaaataatttgataactttgttgccttgcatttatttaaaaaatttttaattctaggctaaaccctttttaaatgaaagtttaacttcttgtgttttcagatactgaatagctatgatacctcttgtgttgagaaaactttaaatttgcataatctgaagttatcttttcttataaacattttattaggtttacagtattgtctttttgttttgttttgtttttagtgaaaaggagacctggaaaacagaatctaaaacaataaaagaggaaaagagaaaacttgaggatcaagtccaacaagatgctataaaagtaaaagaatataatgtaagtaaaacatttttaacattagtatgcaatattgtacaaagtaggatagctagattcaacaagtaatatggatgtgtctttgtgcagaatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggtacatctgattcttattttgctttttctgactatgaaaaatttcaaatatgcagaagataggatggtatcaataatgctcatcacctgaattaatagttaacatttattaacattttgtcataattgcttcttctgatttttgtgggatgtttgaattgcagacattcctcccctaaatatttaatgtacccttttgaaaaaggcttttttctttaactaaccatagtaactttattatacctaacaaaatgacagtaattttctaatatcgcctaataccctgattatagtcacattttttacattttttgatcaaagaataagcatttggatgttacatctcataaatctttttaatatagaatccccttggttttctttttctccaaaaaatgtttgaagatgtatctaacttttgtgtgtgtgtcattttacttgttcctgtgtcccttgtattactaaaagttaggtcagaaccctaagttacattcaggtttaaacatttttggcaagaatacttcataagtagtgttctatactttatattgcatcacttcaagagtatctggttgttccatgttttgtaattgattactctgttaaggaaaagacaagcagaccaagtatggtggctcatgcctataattccaacattttggaaggcccaggcaggaaaatttcctgagcccagaccagcctaggcaatatagtgagactccgtctctacaaaaaatgtttttttttttgtttgtttgtttttaattagcttggtgtagtggcacatgcttgtaatcccagctacctgggacattgaggtgggaggatcgcttgagcccaggaagttgggggctgcagtgagctgtgatcatgtgccactgatctccagcctatgtgcctgtataacagagcgagtctctctcttaaaagaaaaaaagaagaagaagaagaagaaaagataaccatatacctccattattaagcaatttagctaactggtgatattttggtaccatacaaataacaaattatttgtcagtcctaatgattttagcatctgctgatgattgttgcctaacccaattattaaaagttgcaaacatcataattttctagttatattatgcacttacatttattaacagacatgcttttgtaaaataaatagcgtttcctcattagcccaggctatttgtttatcttgaagtttagctcctactacaaaggcaagataaatgcttttctctttaattaccagttttcagaatacacacttggtgtactctgcactacctgctttttttgtcccctccgctttctcttttttaagtatcagattagactcacagatttttaaatattccatgtgttttagttggagtcatattcttttgtctcaactttagccaaagagagtcctttaaagttgactcttatattgtcttgacaaaaattcattagtcttttgaacgaagcctcaaagcttgacttgttttctagcataagatgtcttagacttacctacatacttcatgcccatacttggaataaaccatttctttaaagagcccaggttccttttagtggggaaggcatttagataccaaaaactggccactgggcatcattgctctcagagtatcattgccactagtctctcagtagacaagttagaaaaatatgtatatatttaaaccatgagttcatattgttatttccagtttaattataacattatggggtaagtaaatagtatcggatttttactaagcttctttgattttgcacttgtatttttttcttacatagaaaacctttattattaacattaaaatatttgttttatcctacaatatacatacaataatttgaaaaataatacttgaattgatattaatagtaacaacaacagcactgctgccaaacatagtttaaagttttatttcaggtcttattttcttcagaatatatcttgctgagaatgtataggcaaagtattctacacttacttgaaataattgtcttcatgcggttatgttatacatttgatatatagttaggctcatttgtttttcattttttttattttagggatttttttcctttattgaattttaatatatacaatatttatatatgcaaaatatttaatcagagaaatcttaattctggtcttacgcctttcatattattctgctccaccctctgtaggtaacttattatctttctcatgtttcctttttggaaacataaacaaagacaagacaggttacatgacatgtatacccttctgcacctagttttataccttaccttgtagtttatttttaagcatgtaaatgttcaatgttcatgactaaatttggacaggatcataggaacacagaattcaaagtgaaattaaaatgggcttgggttctttactttccactttaaaggttgtaatgggtgatgtcaggctaataaacctattttcagcttgatctaaagcttaatactgagcatcaagaaattctttaataaatataagtgatatttattcagacatgtaataaggaaatgttcatgtcttatttttgtgttagatttttttagaatctacttttgttagagttttataaatacagttagtgtttgagatagaaagagaaaagaattagttttcttcctcttctacctgctcatgaacttgatttttttctcccaacaattgaagagccaagaaaaagggagattcttaagagatgggaaatagaatctcatctacccctgtttcccccagaacagtgaaactgaatcttaagggtaagatagaatagtgtgtacttaacttagatggagaagaaaggctgccaaaatgagatctgaagcgctattacaaatatttccatcgttactgtacttcagaatgaattacaaccgtaagtttttttacttcctcattcataaatttgattattccttataccacttctcagctttcatcattctttattgtacttttctatgtaatgtttgcctattatacagcaacttaagagaactgtaagtttggacatttcattttggtgttgataatagaatatctttgaatagttctatagttgatgagtagaaccatgaaccaagtaacttaaagtccttgatgttatttattacagagaactataatagaagctctcccgctaatgtttccatcatgtgtacaaaaagttttcttgttattaaagctagtccgtttaacttacaataagcataaatagctaagctgtgaaagttacctgtgataatgctaattttcccatttattaaaaggcaagttgttttccgatcataagaaatttagaaaagccatccaaagataaattccgagtgatatattcctgctgtttgttatgttttctcaaattaattgagttttattttacaatgacaggagttattaaagtattttatttttattatgattaagattttcaaagtaacatttcttatatgaaagaaattatgttaatgcatgtttttcttacatgggaaatcatatattttaaaaatgattttaaaattcgttttactttaagttgtattatctttctcaaaagtggctagtgcttgaccagaaaaaaagacaccagcataactcagtgtatctttatttacataggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggaggtaagtttgtgtgattcttgaaccttgtgaaattagccatttttcttcaatatttttgtgtttggggggatttggcagattttaattaaagtttgcctgcatttatataaatttaacagagatataattatccatattattcattcagtttagttataaatattttgttcccacataacacacacacacacacacaatatattatctatttatagtggctgaatgacttctgaatgattatctagatcattctccttaggtcacttgcatgatttagctgaatcaaacctcttttaaccagacatctaagagaaaaaggagcatgaaacaggtagaatattgtaatcaaaggagggaagcactcattaagtgcccatccctttctcttacccctgtacccagaacaaactattctcccatggtccctggcttttgttccttggaatggatgtagccaacagtagctgaaatattaagggctcttcctggaccatggatgcactctgtaaattctcatcattttttattgtagaataaatgtagaattttaatgtagaataaatttatttaatgtagaataaaaaataaaaaaactagagtagaatatcataagttacaatctgtgaatatggaccagaccctttgtagttatcttacagccacttgaactctataccttttactgaggacagaacaagctcctgatttgttcatcttcctcatcagaaatagaggcttatggattttggattattcttatctaagatcctttcacaggagtagaataagatctaattctattagctcaaaagcttttgctggctcatagagacacattcagtaaatgaaaacgttgttctgagtagctttcaggattcctactaaattatgagtcatgtttatcaatattatttagaagtaatcataatcagtttgctttctgctgcttttgccaaagagaggtgattatgttactttttatagaaaattatgcctatttagtgtggtgataatttatttttttccattctccatgtcctctgtcctatcctctccagcattagaaagtcctaggcaagagacatcttgtggataatgtatcaatgagtgatgtttaacgttatcattttcccaaagagtatttttcatctttcctaaagattttttttttttttttttgagatggagtttcattctgtcacccaggctgagtgcagtggcacgatctcggcttaacgcttactgcatcctctgcctcccagattcaagcagttctcctgcctcagcctctgagtagctgggattacaggtgtgcaccaccacaccagctaatttttttttttttttttttttttttgaggcagagtctcgctctgtcacccaggctggagtgcagtggcgccatcttggctcactgcaagctccacctcccgggttcaggccgttctcctgcctcagcctcctgagtagctggtaccacaggcacccaccatcatgcccggctaattttttgtatttttagtagagatggggtttcaccttgttagccaggatggtgtcgatctcctgaactcgtgatccacccgcctcggcctcctaaagtgctgggattacagatgtgagccaccgcacctggccccagttgtaattgtgaatatctcatacctatccctattggcagtgtcttagttttattttttattatctttattgtggcagccattattcctgtctctatctccagtcttacatcctccttactgccacaagaatgatcattctaaacatgaatcctaccctgtgactcccatgtgactccccgccttaaaaactgtcaaaagctaccggttacctgaagggtaaaagtcaagtcccctacttacctcatgtcatctagagcaagagatgaactagctgagttttctgaccacagtgttctttcttatgtatgttcttttgtacgtgctcttttctatatatagggaaccatttctctcttccagttgttttgctcagtgaatttctattcctgtttcaaaacttgttcaggcattaccttttttttcttaagcatactttttttaatggaacaaagtcactcctgtctacactagttctgcatcttatacataggttttgtacatagtacatatttatatcacatcaaattatatgtgtttacatatctgtcttccttaatggaatataagtcttttgatataaggaactatttaatttgtttctgtgtgttgagtatctcctgtttggcacagagttcaagctaatacatgagagtgattagtggtggagagccacagtgcatgtggtgtcaaatatggtgcttaggaaattattgttgctttttgagaggtaaaggttcatgagactagaggtcacgaaaatcagatttcatgtgtgaagaatggaatagataataaggaaatacaaaaactggatgggtaataaagcaaaagaaaaacttgaaatttgatagtagaagaaaaaagaaatagatgtagattgaggtagaatcaagaagaggattctttttttgttgtttttttttttgaaacagagtctcactgtgttgcccaggctggagtgcagtggagtgatcttggcttactgcaacctctgcctcccaggttcaagcgattcttctgcttcagtctcccgagtagctggaattacaggtgcccaccagcacggccggctaatttagtagagacagggttttgccatgttggccgggctggtctcaaactttggatctcaggtaatccgccagcctcaacttcccaaagtgctgggattacaggcatgagccactgtgcccagcctgtttttttttttttaaaggagaccagtgaagtttcaggaggagggaaagaaaatttagagttactagggagagagtgatgaagataagagatgaaagtggtaataagggaaatagcaaaatatcagggtaggtgggagaaaaagagatttgtaacaaacaataggattatcctgtgaaaaaggatgaaaggaagaaaaaaatggatagaaagatatttaaaacaccctcagcctcctgttttccctcctgtgtattcatagtatataaaactataattatgtactttacttaaaaaatatattattattaccttatcgtgcttatttaatcatagcatgtcctctttttagtctcattaccctgtttgtattattcttcataacacttaatacctgacattgtattatatattggcttattttccaggtactccactcaaatataagttctaggatataatttatttatcactgaaatccattgcttagagtacctggcatgtagtaaataggcattctgttttttcaaataaaaaataaaggaacttaagatatatatttatgttatatcgccagcctttttcctcacagctctattctgttgtacagaattacctactttacaattcctgtgtttcaaggggatctcaaatttaacgtgtccacaatgaactcctgatttctgtttctctcctagtcattcttatttcaatatatgttcagttacctaaccagctagtcaaggcagatactttagagttattctgtagtcattctttttccctaccatttttgttttccaaatgtaatttatgtgtgtcttcttcatcctcgcagctctaacccttgtccaaaccagcatcatcactcatctggagttccacaatgtctttctggctagtttccctgatttctctattgacccctttattctccacagtgcagccagaatgattgtttaaaacttcctccttaaaatctttaaattgttttcttttatacgttaagttaaattccagttccttgtcttggcatgccatgccctgcctggtgtggcccctgatggtctctccaacttcatgttttactactattgactcttatttttgcttactctgcttgggtgctccagtcctccaaatcatttcctgctccaatcatttcaatcattttttcctctcagatcttatagtattccaaatgctttcttcctttggagcatctgggtttactaataaatacttcgtacctcacagttcagcttaaatatcaattatttggtggttaagacatccttcaaccgctctatctaaatgttcctttctattattcactggctcagtactctgtttttattttctttctaaatgtcaacttttttttttttgagtcagggtctcactgttgcccaggctcgagtgcagttgcacaatcatagctcattgcagccttgccctcctgggatcaagtaattctcccacctcagcctccaaaatagctgggattacaggtatgcatcaccatgctcagctaattttttgtgtttttttgtagagatgaggtctcactttgttgcccaggctggtctcaaactcctggactcaagtgattctcccacctcagcctcccaaagtgctggggttacaggtgtgagccactgcacctggtcgatactgacttttttttttttttgagatggagttttgctctgttgcccaggctagagcgcagtggtgtgatctcagctcactgcaacctccacctcccaggttaaagggattcttctgcctcagtctcctgagtagctgggattacaggcaagtgccatcatgactggctaatttttgtatttttagcactatgtttagtactgtgttggccaggcttgtctcgaactcctgacctcaagtgatccacccacctcagcctcccaaagtgctgggattacaggtgtgagccaccgtaatcggccaacattgacatttttagtagactttttgtttgtttacttgcttattatctgctgccttccacactctggcgaaatcctgccacccacccacacacacataggcactgaatgggcagaactctgaaggccagaattttatatttcttttcactgtaaacatcatcatctgtcactgatggcacactaggatgctcagcaactgtgtgcatgaaggaagtaagcactagtttgtgaaggctgcaaaactcttgagtattctaagagttttggccaaaatgaatgtacagctttagtggcagaagctaatactcagaaattgaggccgtatattggataacacaggatttggatgattattttaaaataatattttacattgtatatatgtgtgtgtgtgtgtgtgtgtgtgtgtgtatgtgtgtgtgtgtgtatatatatatgtatgtatgtgtattagtccgttctcatgctgctatgaagaaatacctgagactgggtaatttataaaggaaagaggtttaattgactcacagttccacagagctggggaggcctcagaaaacttaacagttatggcagaaggggaagcaaacacatttttcttcacatggtggccggaattagaagaatgtgagccgagcaaaggggaaagccccttataaaaccatcagacatcgtgagaacttactattatgagaatagcgtgggggaaaccacccccacgattcaattacctcccaccaaatccctcccatgacatatgaggattatgggaactatgattcaagatgagatttgggtagggacacagccaaaccatatcagtatgtatatgtatacaagtattatatatatatgtatgtgtttgtatgcatacatgtattatatatggaggaaattctaattttgtaaaaaactggattgtgagttttaaggagatgttatataaagttaagacaatgtcattttgtggtattggtctgaattacaatgtagtttcttagtgatatttttcctttattcagtgtgaaaacatctccttaaaagaacaagtggagtctataaataaagaactggagattaccaaggaaaaacttcacactattgaacaagcctgggaacaggaaactaaattaggtaagttttatgactctgataatataaaatgattaacatctaataatgaatatttcttatttaaagttccttttttatgctagattaaaaggaagtattttgactaaaaaaagaaagaactttctgcctaataatttaacttaggcagatgaataatcctgtacttaaccccaccaaagtttagttttcagtccttaagttagatttgtttctaatgaaatcatatatgttaaaaatttatgactaagtattagctactttgaaccgtttaacaattaaaactgatgatattttattaatggtattatgagttctttcactgagtgcaagttatattagttatatatcacttgatatttttaaattaaaagataccaggaaacagcaaagaaaatgtgaaaagaagttgtatttctcatagttttactactatattactgtatatttttgctcctatatgcttacatattttatatattttaaattattataaacatggttttatactgtatttagatagtaatatcaaaaatatttttatggccggcgcagtggctcacacctgtaattccagcacttgggaggctgaggagagcagatcccctggggtcaggagttcgagaccagcctggccaacatggcaaaaccccatctctactaaaagtacaaaaattagccaggcgtggtggcagttgcctgtaattccatctactcaggaggctgaggcaggagaattgcttgaacctaggagtcagaggttgcagtgagccaagatcataccacagcactccagcctaggcgataagagtgagactccgtctcaaaaaaaaaaaaaaatttgttttattcatcatacttataaatacttatacaatagcctaatgtgtttgagtgattaaatcactagctttttatatttttgctattgcttatagtgccacagtgaacattttcatgtatatctaacagagatattactgtctcagaaggtattgaaatctttgttgctctcattagagttttccatattaatttttcaaacagttatatagtttataagattttcataattttatctcatatattgtgcttcataattttcaaataaatttgctgctttcgataatgtattttcatgtatttgtttcctagacgttagagctattcaaggtttttattactaaatagagctgttctcttaaattggtaatgagatacttggtttagagaagcctaacactgggaaatcttacataagctacttttagaaatgtaatttttagctcaataagagattaaatatgaattgacttttgtgtagtatttgcatggaagaaggtaccatttaaatgaagacatgagagtattacgtacaattttagtaggttctttttattttatcatctttatttttaataaatgctgaattccctacagaaattctttaatttttacatatcttgatctctttcatatatggatttatatcaccgaagttttaagagtgtttccctattccctgttgcccttatatctttgtttaaaaatgtcacatcattagctttttttcatctaggaatttgttagtgttgggctgttgtgctctaccctctctttaagaaaactccaaacccaaaaacatacaagatggctagtctgcttcagcctttgtgatgtgcttttctcttctaatcagagtttagcacaatacagaatggagaaggactcctttatatattggtatttattgcagtatttttctacatggtgcctaaggttacttgaatgagtctttattccataatgaactgatttactaatgcttttagcacctgttagtgatccattattgttagttacttgattactgcttgccacagctattctaaaataatacattttaaagataaatacagaacataatgaagtactttttaaaactgagatagagaccaatttttttttcaggaaatgtatattactttgagaaaactcagttataaaacttgaacttatgaagctggaaaaacaggagggggcattattggtattgtaaaaggctgtttacaaagtgagttgctgcttagttcctttaagtaattggctaccctaaacacatcagttttaagttgctgaaaagcaaaacactctaccaaattttgttttttttctagaccatgtttacaaagcaaaagtatgttttcttccccccccctcaaaaaatgactaatgacactcctatgcgatgcctttttatggtaaattgaggcttttagttctctttccatttagccacagacttttgtgtccaaagacaagctgcgtaactgcatatataaggttaaggcataactactaataaaagaatgtaaaatatttgatattaggtctgtacaaagaccaaataatactcatgattagacaagattatatttggtagaatctatccatcatatggcttcagattttacttttcagcttggctttgtgagactttaaaaatcaagtcattgcacttatattcacaaagtcacattgctttactgcattgcttctcatacagtttatctcctttcagtaaaatgtttacttgccatttttaaaatttcttatatgtgacacttctacactaagtcctttatgttgttagttccacaattctgtgaggaataggtttttttttttaatcatttgattgatgaagaacattaagttccacagagattaaatggtacaggcatcacacaggcaggaagtaacagagctaagattagagtccaggtctgatggaattcagaaagctaatgtgctttccatggaactataatgctttctaatatacagcatctaaaatatctgaggtaattttaatataaacagcatgagattgacttaaatattattgcatgtaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgaggtttgatattataagttttatcatacaattatagaataaagaattagttttggtagacattgtattattgttaagtggtttgtctggatctctgaaatatcttattaatatagtgcctatgttttgtgtaataaataaataaaagatttaaatctgaattgtttaaaaggaaagcagatatttctgtaagtttttctcaccaatgttatattattagatttaatttatgaaatgttatttactaaacaatggaattgcctttcaccaccatcccttcatttaacaaatatttattcattgcctattacatgtcagaccctgtgttgggactggcagtatagcaagaaacaaaatagacaataatctctactttcagggactttacattctaattggtggttttatatatttttgatgtggtcagaatcattaaactgtgtggcagtaaatatagtttgcaagtatttaacaatttatgattaaacacaactcttacagtgtttgcttaccttgagatttaatatattttcaaagcatttatatcatttttgttttaactatgtcactaaatctatatgagtaagattttattaactcatttggatttatttatagatgatacaattgaagtaaaatataatgagcagattgcattctaagcaaagtaagaatattgcaagttcagatattattagataatgagttgcctaataaaaatgacttttggtggattggaatataaccagagtttccatagtttgtttctgattctttcatattttttaccctccttcagtctgttcttaacacttcacacttaatataatatgtgaactaaggccaagtaaagaggattgcagtactttaaaagctaaattacaaagaaaacctcaccaaaaattgatgtatctgaacattttttgttacatttccttagcttaccaaaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaagtaagtgcatataagcattttagccatttgactagatgtatcttctttaatttgtctttaagaaacccaattacaggtatacaattcttagtagtaattgatactgatttctttttataagaacaggattaagtaatattaagatcggttttaacagggttaaataataatattgacgagaataatattgttaaagaggaagtgacctctcaagatttgcattttttagagttcaggaatattattgcagaaaggtccagttcctccacatattgattttttggggaaggggtgatggaggaggaatggttgtttattgtatttaaacttaagtttcttcattttaataagggagtaatagtacctcttctacctgtttcataaggttgctgtaagaatataataaaaaattcagattttgatttagtttacatttatcgggcatctactatgtactagtcacggtgcaaggtattaaacatatattgacttgtacaattatacttaaccttgaggttatatttttgttttcattttacatgaagaaatatgcccagctagtttagaacacaaaatatatataaggagtaaatactgcgtgctggctgggcgtggtgacatgtgcctgtagccccagctactcgggaggcagaggcaggagaatcgcttgatcccgggaggtggaggttgcagtgagccgagatcgcgccactgcactcctgcctggtgacagagcgagactctgtcaaacaaacaaacaaacaaagaaaaacaaaacaaaaaaaccgtgtgccagctatatgctgtattttcattctcttttgtaattaggtgatatttcagtagaaaagtataaggagcacttagttaatctgtcaagcataaatagtaaaaatattttatggcctactcataaaaatataaccattcctttggagccttgatagttctcttgggaatatcagtttttgacatctttttcactatgaaagacccttttttttaaaaaaattgatcctttcttctcatggacctcttttgatataaactaacttataatagttcattttaatcatattttgttaatcatgcaactggcaatgagagcctctcatcagtatgaggaaacctgccttatctataatactgaactaaaattattctaacccaaagcaaagaaactttacattttgctttgcctgtattagcttatcacagtattcatgagggaatttgaaggacttattaccattaggctatctctttttttttttttttgtaattttattaaatgcatgttttgtttcttttcacattactgataacttgtagattaaaacaaatcaaaacatgcattaatccatctaaggatcctagaaattttacatttctgtgttcttaactgtgtgatggtcttagataaatgtactaaataccttatcctagcatattccaaattatgacaataaatgttttatggaaaaaagtatgggaacagaagttctttggctatatacatttggaaaatactatatagtaagtatgatttgagataattatatatgatagaacctctgggagcactgaatatatgttaggaatattcaagagggaggagggatgttgagaatgaagttttttttatatagcaaacatgataacctctgatggaattatgtttcatgaaacagtttaggaaatcctgttttaatatttcatacaaagaagagatagatgctgaaaacgaatggctttttgaaaaagggtctagaaattttgaattttggcatttacttagaaagtgtacttaattgttcctgaaataccttatcatttcctagactgagagagatttctgatattgccagaagacaagttgaaattttgaatgcacaacaacaatctagggacaaggaagtagagtccctcagaatgcaactgctagactatcaggtatgtgcagtattggctcttctacatagaatccacttttttccctaaatttacattagatgttgggagtgggatatgttatactttttgtttgtttcgagatagggtctcattctgttgcccagggtggagtgcagtggtacattcaaggctcattgcagccttcaccacctgggttcaggtgatcctcccacctcagcctcttagacagctgggactacaggcacgtgccaccacacctaatttttttgcattttttgtagagacagggtttcaccatgttgcctaggctggtcccaaactcctgggttaaaatgatctgcccaccttgacttcccagaatgctgggattacaggtatgagccaccatgctgggccattgttacatttttaatcaaaagatataccaaccagaggctgttattcttgttagttggaacctgattagaaagctctttaatttgaaatattgttcagtaatccagtacagcatttaaatgcctatagatgaattatgctgctgatcaaaattaggacactgagaattgtagttagtaaatctttaataacaatattttctcttgtatttatatgtaactttttacatattcttacgttatatatgttgggaattataaaaacatacacattgtcctgatcagtattatgttacttgcaatggaggttaaaaaaaaactgtaacagtcaggcatggtggctcacgcctgtaatcccagcactctgggaggccgaggcaggcggatcacgaggtcaggagttcgagaccagcctgaccaatatggtgaaaccccgtccctactaaaaatacaaaagttagccaggcgtggtggcatgtgcctgtaatcccagctacccaggaggctgaggcaggagaattgcttgaacccgggaggtggaggttgcagtgagccaaaatcacgccattgcactccagcttgggtgacagagtgaaactctgtctcaaaaaaaaaaaaaaaaaacaccagtaacatacccactgttattcagttacatttggattttaagtttgtttgattctaggttttttcttttacagttctttggtaattatttgtattaaagcaaagttacatttttgtagatctcatgtgccactgtgttaaaactttgcttagtaaattgtgaattttaaatctgtgataactttcactggaaaaatttgaaacttactacaaatatatattttttttaatatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaaacatttaaacttgatttttttttttaagagacagtatcttgatctgtttcccaggctggagttcagtggtgcaaacatagctggaactcctgggctcaagggactctctagcctcagcctcctgagtagttgtagctggcagtacaggtgcacaccaccatacctacctaattttttaaaatttttaaatttttttgtagagacaaggtctcactttgtcacccaggctggccttgaactcctggcttcaagtaatcctcctgctttggtctctcaaaagtgctgagattacaggcatgagccactgtgcccagccaattttaaattcattatcttcaaaagagttacatgataatttcttaatatatgcctatatgaaaaatgcttaagatacaaattccaattatgattcattaatttagattttataacttagcagtgttggctatttgaatgtctattatacgtaaaaataaaattaggcttttctaaccaaagattttagtgggaatgttcagattgtataatagcaaagaattttaattactataggaaaatttatattaattaaacactaattattatatttaaacattgtagtagttatcagttgatttctactgttcataattatctttgatctacaagtagtgggcccacatttacttttaatatggtttaatcttcatttagaaagaattaaatgaaaaataattatcttgcaactacatcctgttctctaggctagaaacatttaggatttctgtttttgaaagtaataccaaagttccaatgacctgcttatagtcagtgttcaataaacgtataacaaatgaaagtgaatattagtgatgtccattccaacataatttgaagatttttattgtaaaatcccacatatttgtagaaaagtctatggaaatcctaaataagattttgtcatgtagtttgacaaaagataacattgtgtcttattttattttagaatggccattactttcaattaaaatcattatcatcaatggaggaatgttatttgttaatatagcatttatatttgtgtatataaattgtaaatcttaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaaggttttattttatatttatttcatttttttccctaagttttttttttttttttttttttttgagatggagtctcactctgtcgcccagactggagtgcagtggcgtgatctcggctcactgcaagctctgcctcccgggttcatgccattctcctgcctcagcctcccaagtagctgggactacaggcacccgccaccgtgcctggctaattttttgtatttttagtagagacggggtttcaccatattagccaggatggtcttgatctcctgacctcatgatccgcccgcctcggcctcccaaagtgctgggattacaggcgtgagcccctaagattttaaacaagaatattgcacaaatgactatgttatccttctaattaagtgcaccttccattactaattgattatataataatttgttttttattttctaaactattctaaaaattcatatttatttagcttttataacagtagtcttaatcttaaaaacggcaatacataagcaacctcatttggtaagttaatttttattttgatattggttatttgacttttcacagttccacgtttctactggctctcactgatagagtaagaagtcagcttcttatagaataaagtatatacttcagagacagatgaaattcgtcaaacatatgactgtctcagagattgttccccctgcttaaattgttcttaccctagatacctttggtatttacactgtcagtgcctgcaggtcttagctcaaatgtcttaccttatcagtgtatccttcaccagccacctaatatacaacagtaaatcctactatccagattcctaaatagagattaattaacttaatttttctccaaagtgcttgtaaccttctgacgtattacatacttactggtttattattgactgtctttccttcgccagaatgcaagttccgtggtgacacggacttggttttgtttactgccatgtttgtatttcctagaatgatgcttggcacataatatatgtcatcaaatatctttcgtatagctgaacggatggatggatggatggatggatggatggatagactgaaatccttacttcacatctgcctttgtgatcttacacaagttacttcacctctctgagtttgtatttttttccataaaaggaaaataattacagtttcttcaatgtgttgtgaggattagataagaaaatatatataaaatgcctgttatgtgcctgatgtcttcgtgtatgtgtctgacacaaattgtcctttttttagtttcatgaagaaagacaaatggcctgggatcaaagagaagttgacctggaacgccaactagacatttttgaccgtcagcaaaatgaaatactaaatgcggcacaaaaggtatgaatgattaatcttgtttgttactctgtagcatagtctagagtgttaactcacagaaatatttcctgtatcagatgtaattttaattgatgttatattgtatatttaaaatataagaggggtttaatctatgttttatcatacagctgtaaaaattaatagttactctcaatgctgcaactgcttttttaaaaaacatactatttcttaatagtttgaagaagctacaggatcaatccctgaccctagtttgccccttccaaatcaacttgagatcgctctaaggaaaattaaggagaacattcgaataattctagaaacacgggcaacttgcaaatcactagaagaggtaattagaagaatttgcattttgattagtgtattatttggtatgtttggggggctttctaaataatatttctttatgagggcaatgcatagaatgatgaatctattgctaatttcactatttttctattctcctataatgtttctaatagccaataatgaacagcagatatagttaatttgaattcactatttaattattagttggtacctttcggtacactgaatatgaaaggaaataaaaagcatttaattgtagttctatgagcaatatattctcttatatgatctctttattcttacttttttggttttattttgaagtgcatgttacataatctatgaatcaattttcagttcattgcctttaatgcatggttaaagggttgaaggtaaattagaaattactttctgttttaacctagatcttgaatttgattagtaggtgatcaaatctgtcatcttcattaaattattcagaaaataatgtaaactgaatgtgttttcattttagttttcatctaaataaactgcaaatacatttaaaatatacataaagaagtttttcaagtaaaactgtacatttttaatcatttcaggaaacgtagattttcttcagtaattttaagatttgtcatttatgtgaattgccattgaattacttaatttaaaatactcaccttaatcctcttgaagagtaaaaatttttctgtttttttctctttgttttaataagctgcggattttatattcgtaatttattgagttgggcctctaaaattccagttttgtacttaactgacttatagattagtctcctaatgctctgctagtcaatggaccaaaataaaagaaataatttattacatattcttcctaaatctagtaccaccatacatgtataattctaaactgtaatatctcaataaagtaccttaattaaattttatgttcatcataacaatgaagtttctagcatatgtaatagtcttataaataagcatgcaaataactgctgtcaattagaattagtcagtttaaccttattaagtatcaaatggctattgtacatatgatgtgaaaaataaagtgaattttttttggctaataactaatctaaaattcagatgaagcattttaaagggaaaaagatactttaatgatttattataatttaatcattgcagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggtattttattatattatgagttatgctgttatccattagtttttttaagcaaatgctaaatattattttaccctaaagtggtatttcttttcttgctttcaaatgattctatttaagaattgttacttgcatgtgattggattacacctctgtcagtaaaactggaagtttgtgtacatgtatctttctattatacactgactaaaccacgagtagctatcatggtgaaatcatatgattttgaaaaatattttaattgagtttataggtgaggattgaggcaatagggtggaatgaaatatatcacaccggtaatcagtagaaatcagatttgttagaacttcgtgggggaaagctaacatttaattttttctagaagtaagttaaaagatgatagatacatgtcattctaatgttaagaataaattatgaactgaggctgggcttgtcaacttgaacattgtctgaggggacatgcataccagtctagatacatacatatatggagatactgtttcttcctcatctcaaaggaattttagaagattgaagagaaaatatataaggtcttcaaaatgtgaatttgttttaatcacaatttaagatatagtttcgattttctgtaaaacaggagcaaagagaaattgtgaagaaacatgaggaagaccttcatattcttcatcacagattagaactacaggctgatagttcactaaataaattcaaacaaacggcttgggtaagattctaagaactttgttccattctttattgatttttgtgaccatgtaaattaaaattcagctctcttcttttttggaatggaagttaccctttttgttgccaaaataatcttctgaaaacatagctctgatcattcttcctcctgtagctcaccgctgttcacaaaattatatttataattcttagccatgtactcaatctgctatgaacctacctgcctttcttttcaaattctactcactgtgagtttagctatatctaacttccagaattcagctcatatttgcctcttttgaccattctgttccatatgtatgaaatgacatgtctttcatcttttaatgtgtaaccttagcatatttgagcattacctcgttaattcggtcaacacttattgatctcctgctacgtgcagacattttgctagctattgtaaatacaaataataaagtctgcatttcctgtcttctttaagccttcattgcctattaaatcattacattttagattagatattatattttgatcatttgaggaaccaaattaaaaatatggaataagtatggcattgaattatacatgcctattgctaatatattcatattttataggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagtaagtcttcgaaatgtattgtaaaaataggcaaatgataagtgatataatgaagataaacataagtgtttgctatgccaggcactgttctaagacttttaagtatattgtctcatttttatcctcaggactgctggttacatatgttatcattttccccattttaaagagaggatatggcctcaggaatgcttaatagcatgtctgggggtagatgggaaagccataatttgaaactagtcagtctgactcaaaagccaatacaaattcttttccagaatctcatttttaccttctttgagcctcagtttcatcttatttatttatttttatttttgagacaaggtctggctctatttcctaggctggagtgcagtgacataatctcagctcactgcaaccttgaccttccaggctcaaaccatcttcccacctcagcctgcagagtagctggcactacaggcaggtgccaccacacctgggtagtttttttgtatttttgtagagacaaggtttctccatgttgcccaggctggtcttgaactcgtgagctcaagtgatccgcccacttcggcctcccaaagtgctgggattacaggcctgagccattgcacccagcctcatcatctttaaaatggaaataataatacttaccctggccctttcagggtggttatatgaaggtcaaattataccgtgtatgaaagtaatttgaaaactgtaaaataacatacagatagaaaacttttgattacacacttataagagtgtctgtcatataatagagattctaaacattgttcaaccactttatcagaacgtagattttaaactcaaaataggtttatagttaggtagtttctaatcattataatattatctctatgggcctaaattttattatctgaaaaaacatgagaaaattgaactgcttgacttataattccatttcagctctcaagcccctgctagagtctttgattctttactcacttattcaaatgcctctgacagaattaacactatttttgctttgctaaggagctgccactgttaagaaattactctctaaaagaaagaaaattggcaacagcatatgtgtattttcagtctcttttcctcactctattaaattttgtacaagagatgttatttttggtctagtaaatttctgtcatgttttggagtataaaattacttgtgcttttgcatctaatttgtgggtgtagaaaatcataatcttttgaaataccttatataatacatttttttgccacaggaaatacttgaagttattgttgtgtaccttacgtcattttagtccaaaattatacttgtgttctctgtgtgcatattttgatatgtattaggagattatggatctgtgtgatttcttaagtaaatcctgatattttcacaatttgatgatgactctttaaagttagacttaagttttgccaaaagcaagaagcctcaaagagtaacatttgttcatgtcttaacactatctccctcttattggtcagaatctcagtatggatgcagtgtccatatgcacaacaatatattaattcagtttaacagacttaatgctgaataagcaataagattaattgaattaactaaatcttttgatagtatccacttccatatatatagttatagatataatgctagtgaatttgaaccataaacaaattaataatacatgtgatttctgtgaaaatttatattagtcttttcaatatgtcaatatagggcagtatttctcaaatatagaggatcagtttttcaccattgtccctcttggggacatttggcgatgtctggagacatttttgattgtcatggctcgggggtgctactggtatccagtgggtagaatcaaaagatgctgctaaacatcctatcatgcacaaggcagccccaccaccaacaaagaattatccagtcaaaaatgttactagtagtatggttaggaaactatcatatagaggaagcaatcacattttacaagagccataatatttaaaatgcctttttgttcattctctgtatatttgactagagtcacaaaataacttgataagattgttgccaaaaatattagaaactagaagaaaaatgtgttgttaagtctaagagtagttaaatgaaataaagaattattcttctttggatttggatgcctgcatcaagatttagattgtaaggatacttaggactgaacatttgctctatatgaaatttgtattaatcaaggtatgaattgcagcaaccactctattaattacatatgtttggccaggtgtggtggctcacacctgtaatcccagcaatttgggatgccaaagcgggcttatcacctgaggtcatgcgttcaaactggcctggccaacatggtgaaaccccatctctactaaaaatacaaaaattagctgggcctgatggtgcacgcccgtagtcccagctactcaggaagttgaggcaaaaaaatcacttgaatctgggaggcagaggttgcagtcagccgagattgcgctgctgcactccagcctgggtgacagagtgagactgggtctcaaaaaaattaaaaattaaaaaacacacacacacatatgtttatttacatcaggcttcaagaaaaccatgaagatgaagtgaaaaaagtaaaagcggaagtagaggatttaaagtatcttctggaccagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagtaagtaacaacagaaaattatcaacatttaggaaaaatatgtggtagattgcttttagagaagatttgtaaatttataaaagatggtagtataaatctccgtgttgtaataaaaagtatgagctttatcttatgctgttaaacaaggtattttagacaatgctgtttttgtgggcagatatagtccaatttatctttttatgttttcgtcaatctgatttgtgaattatctatatgaagttaggaaaaatcttaatgtacattacaaaaatataatatatattacattgtattttctttttttctactggaattttatgctactgaggctatttttaacaaatgaacaattttgaacaatttgagggattgagggaagtatgataatgacaaaaagggatgaaaaaagggggtcatagagatgtttttgtgagaaggagttggtcagtgtattctgatttattagggttttttttagtttatctcagatttgatctatttaaattgttttagaagatgctggtgtttttctgtgctagctatgaaatttatgggtaaactttaagcctttcctagtccttttgttgtctacctaaattcaattaatttcatatggaaggatgtagtaagtgagtaatataaatatctaaaattggatgtttgaaaacaaaacatacctgttttttgtaatagcttgatttaatgctgagttctcaaaatcattattaagattttgaactttcacattcaatgtggaaagaattgagtgtaattacaaaagatttatttgaaaaagttgagttgttaatttgtgaaatatgttccattaaactcataatattttagaaaaatagtaggaagtaataaagcttgtttattttttatatcatatattcatataaaatgtcagttttcctttaaaaattacattttttttttggttaatttttaggcacttagtcgggcacttttagaactccgggcagaaatgacagcagctgctgaagaacgtattatttctgcaacttctcaaaaagaggcccatctcaatgttcaacaaatcgttgatcgacatactagagagctaaaggtgaacatcaacacgtgttaatgtaacaaaatttctgataattcctattggaagagaattcactatgatatatagtaattttgttgatgaatagggaatttataatgcactgttggtggctagacatagacacacacatgcatttttcaacaataagtctctttatgatactcatttactgattatcatcttggggattaggaaaggataggccattatgaactactgtttctaatgaaattaaatttaagaaatattttacttaggattttttttaagactttattatttttttagagcaattttaggttcacagcaaaattgagaggaaggtacagagatttcctgtatatctcctaccctgaaagtggtacatttgttaaaattgatgaacctatattgatacatcataatcacccaaagtccaagtttacctctattttagctcttggtattttacactctgtgtgtttagacaaatgtataatgatatgtatccatcattatagtattatacagggtattttcactgccctaaaaatcttctgtgcctctcttcttcattccttcctctgcacctcaccaaacccctggcaaccagtgatctttttactgtctccatagtttcaccttttccagaatatgttatagatggaaacatacagtgtgtccccatcattctcaccataggacagctaggaactcctttctagtggcatacatattgtctagtattgtaagttacccttttatatcttatctttgtaaactaggttagaaattacttcaagtcagagatttgttctgtactactcttatgcttcatagtgtttaaaacgttgtcatatatattgttatatacttgtttgtttaattaattcagccaaaatgaaacgtgcatatttgataaaattttgtttgtgggtgtttgttgaagatgaattgctttacactagttttttttttttttctcaaagtcgacttttttcctcaaggtagacttgacatgaatatggaaaaatatatgtagtttgtggttattttttttctcttgtgtacttaaaaattcagactgaatttttcttataatggtatattttctgttttatgttccttttatcattgatacttcttgaagagtcatgaataatacctttctttttctcttattagacacaagttgaagatttaaatgaaaatcttttaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacaggtaattttatatttaactctgataatgtctgatttacaatatagaggtagtagtttatttctactttatcattttatctatggtatttgttaaaactgactttcaaatcactttgattaatgtaattaatttcttttgtgacttctattgtgtttatagttctagagtagcatattagtatgttgtattaaaatgcagaagcagctaccagattatcttatgtattaagtgtcatttagaaagtatggtcagtgatagcttcagaaagttgctattatataattgaaatatttactgtctattttgttttacatttatttgtaaaaatataaagttacattttattttttagggcaaacccctgacagataataaacaaagtctaattgaagaactccaaaggaaagttaaaaaactagagaaccaattagagggaaaggtggaggaagtagacctaaaacctatgaaagaaaaggtatgtgaagaaacatactgacttatatgcttaaggtagtgacagagtaagttaaatacatagctgattaacagttaatatactgccttaatttgatgacctggctgtattaattctgtattaattttgaggactataagcagtattgaataacgtagaaaagtctaagtttctgttctgtaggaatttagagtctacttgaggagatacctataatgtaactcttatttggaaattactacatcaatttcattcatctttctgacattagagtacctctgaagttccttcacaccttaacatattcaactgtgtatcatttctctccaaagtaatcatttacacaggttggtgcttttgacttttgggacagaaagatagacattttaagataccccactttgacccaaataggtcctttttaatccttcaggagactaggctgttatttcagatagcaaagttatttggaatatcttcagtatttgcagtaataatcagtaaccaatctgctcatagattaattctgtgggagaaattgcttaaaattttatagttcatagtaaactgttttgtaataaaaattactgattgaaataaccccaaaaaaaactaaaattggctaaaatgcgtgtaattaaatttgttatggacaataaattggagataacttgttggtaacattcaaaatatcgaaagtgaactgggaaatgttgatgttagcagtaatatttgccattgaagaaaatcagtatggaggagctatggttaggaaaatttttattataaaatttacccagaaaatatttaatgtctataaaataatttcaatcacatgaaaatggaaaagaaaattctgtctttaaaggcattgaatagaaaataggtaatggaattcaaatttcttaatagagtatgctcccaaaattattttctatgaaaattcattaatgtcagtgtaatttattgacactatttgcgtggagtcacaacatgcttgctgtcagaagctttgctggtgaaaactgtaagatcaaagtgtccttaatcttttggatttccatctttctaactccctaattggggataggcctgatcttatccctaaatggggataggttagaaactggtatgtttgttcctaactggtgtgtttctataccagtttctaacctgattcctatcagaatgttttaagagccttgtggctttgcctggactcttctatgctacagtttatttagtttatttattcagtttattcctccttaaagtgggaataatactatctgtattgccagtttctcaggattattttacataaaatgatatgatatgcggaagtcttttgtaagccatcacatccatagcagtataagatattactactaactagaaagagaaaacaggggtctatgcccagtattaaaattggcattcaggaatctagtgagaatattttttcaggttcattgcttgggcatttctaatttatactcaagaaatgctttcatattgtttggaaattttagtacccttttctctgtaaacagaatttgtagtctacctatgtaacaaaacccacccctgtgccttgcatttcattctccttagcatttattactatcttaacatactagacatgtacttgtcttttgttcatcttttttttttctttttttattagaccataaactttgatggcaggaactttgcctattttatttattattgtattcccagcacctagaacaatcgctggcacatagtagatgctcagtatttgttgaatgaatataaatttttaaatgttataataatattattctgaaatctatgcatacgaagcttttggtacagaaaacatgaaaagagaactactgccttatcatccagtcttcttccctcttctcattcagtctagaacataacctgttttggaaaaagttctcaaaccatatgtttatcttgccctcaaaccataacaacaatcaatgcaaaagacttctgtgacccccagaatatgtggggatttctccacatcagcaagcaagcagttggttttgtagcagacaccaactgggtgtcgtccaattcaattcatcatctacctggagatagtgtcagatcccacagatatcttacttcgatcaaatcacaagtccaggcctccgtgacttccgaagttcccacatccccagcccccagctttgggtttgattaatttcctggagtggctcacagaactcagggaaacatttacttacatttaccagtttataataaaggttattacaaaggatacaggttaagagatgtgtaagaagagatatgggggaaggggtgtggaccttccatgcctttctggggtgccaccttcctctagaaacctccacatgttcagttctccagaacctctctgaacccagtcctcttggtttttagggaagcttcatgacatcagtatttcttctcctagggtatggggcaggaccccctcgtattagggttttaagacccacagtcagaaaggcaggggaagattacagtcctgccttagggcaggtgaaaggaggatgggagaaggtcagagagactcttttctgaggtgtgctcggaaggcctaacacactcaatattataactaaagatgaggacaagggctatgagagttataagccaggaaccatggaaaaaagcctatatgtaataacaccacaatacccatggtaccattcacgtttgttgtttttctgtttttcaattgttctttcagtcttggttcccttaatcttaatttagcaagtaatgccaggtgggataaaattgcccaaacccaacaaagtactgtgtgctgcaggattatttaatgacataccttatgtcccccactagtatttacatttctgggagtacagaaaaattcttgtacatatttcagaaaaaatgaaattaataactatcaaccacttagtgaagtttttacttttttttttgagatggagttttattcttgtcacccaggctggagtgcaatggcgcaatctcagctcactgcaacctccgcctcctgggttcaagtgattctcctgcatcaacctcccaagtagctgggattacaggtgcctggcaccacgactggctaatttttgaatttttagtaaagatggggtttcaccatgttggccaggctagtctcaaactcctgacctcaggtgatctgcccgccttggccccccaaagtgctggattacaggtatgagccaccacacccagactgaagtttttacattttttaaagggcacttattagctgaattaaataaggtaaaaaattgactagtattagagacaagaattggagaatatagttctctagtattcgagaaagtcgttttgataggacaactaatcttagtgagaatttggctttatttcatatttttttaattttttgagatgacgtcttactatgttgccctggctggtctttgaactctgggctcaaacaatcttcctgcctcggcctcccaaagtgctgagattataagcatgagccatctccccaggaatttgactttaaaccatggttctcaaccctttcagattcaacattccctttaataaaaaatataatgtttcataatttcccctttactattataattgaaatgcatagttaacataaactctacctacttacataatttcaaaaatgtcattatgaatgtcctaaatgaaatatatagggggaacataaaaggaatattcatatttcaacatgtaaatgctttggcatgactccattggaaaatataatgaactagtcatgtgcttgcaccttcattaatgtgagttcaaagctacgattgcagactgacacaaatgtgttctattggcaactgatgggtcatgatggtattgccatttgtaatttgatttccaaaatggtaaacaaattgttggtgcagttctcagcaaaacaatgtctataatcttaccttttataagactgttgtattcctagaaaacttagtgtatagtaaaaccattaaaaaattacttagtgtgaatatgttagttggagataaattcttagctcagaccagtgtaagcagaattttttactgtattaatatccagtagaacatttgaaagttgttcagtgcatgagactattctgcattggataggctttctttggctcctttatcatagttataataaaccatgacacctacccctgaaatgccctaattcccttccgtttctttttcttttttctttttagcacttaaaactagctaacttactacaaaatagatttagatttatttcttgttttgttatctgtatcgtttgctcccttctccccaatctatctaaccaactagtataaactagatagtaagattcatgaagatacacttttttatctgattttattcatttgttctattcctattgcctctagagtagtacttggcacatggttagcactaaataagtacctgtcaaatgagtgaagtaatgtgcattgaagacttgaaggggctctgatgctaggaaattgtcatgggataatagatgaggttggtcgtttgtacagaggattcttgttagaagcttactctagtcatgattgtattagaatcttcatttaaaggctcctgaagggtgttggcattagtcagaactgtctcccagaattttatttgtcttgtgatagaataaagcatagttagcctaaagagcagttttcctaatagctcggcatgcccaaagattctaggagttatacaggttgaacatctaatccaaaaatctgaaatgctccaagatacaaaattttttgagcaccaatatgatgccacaagtggaaaattctgatgtgacctcatatgatgagtcacagtcaaaacacagtcaaaactttgtttcatgtacaaaattattaaaaaatattgtataatactacctccaagctatgtgtagaaggtgtatgtgaaacataagtgaattttgtgtttggacttgggacccatccctaagatatctcattatgtatatgcaaatattccaaaaatattttttaaaaaaatccaaattctaaaacacggctggttccaagcgtttcgtaagggatactcaacctgtatagcaaaatgaacatatttacatattctctaggaaatattagtttacaatttttctaggcaaattataattgataaatcataaagaaaatttaaaataacactggtaattttcctacctccttcgttattgttacagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaagtgagtttaaatatcattataaaactaattatgtgtaaaatcctttagtgacctggaaattatatagctttatcatagttgataatatgagaaatggtctagtttaaatgatcatttattatctatgatttacttactttttattttctttaaaatctgttttaaatatattgtaacaattatagatggattttcctgtgatctcgttgtaaattagcttatgacaaatatagggtgttacaattattgtaatttggtttggtaatgagtatgcaattgaaaagccaaacactgaatggtatatttcatgattctatattaaattccacagagccgataaagagaaacttactttgcagaggaaactaaaaacaactggcatgactgttgatcaggttttgggaatacgagctttggagtcagaaaaagaattggaagaattaaaaaagagaaatcttgacttagaaaatgatatattgtatatgaggtaagctattatgtggaaatgtgccacccattgtaatgaaaaactggttgacccctagaaattgaaataataaatgtgtgttgtcttaagcttgggttatgttttcttttcccatgtgaattgagatattcctggttcttcatatgccacataattttggtgtatttttgatcttttgaatattatattgtgagactctggttcttgtttaaattctatgggaaaatgtagatacttttgttttagcatgcaatcggtctaattaggttcaggccacaagttccaacctcatttcttgggctgtggttccatttttcaaagccttttcaatactcttcagatctgtcctgcctgtgtacctcacaataggtgatctggtatgtgagctatgtaccattagttcagttcttagaaactttggtattctgattaggatcgatccatacatttgcagctcaagagtgagcccagaagttcataaacaactttatagggtccctttcttgagctcctccctctttgccatctctctgatactttgtttccctagggatttccatttggggctttagttacccagtgatgccatgtacttcaggaattgcacacttctgcagccaagcaagcaagaggagagtagaaagaggaagaaaaaaacgacttttaccttaccctcttagtatcatagctctaccaattggagatttccctcccaaaaaatattagcttctgtgagttcccattgcagcctctattaccactgctatgggatggcttaagggttggggcatgaaagaacagatagaagaaaaaaaaagtgaggtgttttcatattgtctcttgagtgttaaaagattccctttctctttactcgagctagaattagaaggtttacctggagctctctctgtcagtgcagacacccatcttcaggtttcaaataatgttgtcttcagggcaggcagtaacagaataaaagaaaaggtaaattcatcacctgtttgctgctactttaagtcctggtattctattgtaatctgccttctactcctttgcaaagtcctcaaatggttgctccatgcatttaggagagagaagattgaatgtatttactccattgtacctggaaccagatgcccttgccctgcatcaccccatgtcatttcttagcagagcctttgagatttttgtgtgtgtgtgctttacaatctctttccaagttatatcttctgatacagtcatggtcgtgaaaagcaaaataaaatcatgtgttaacatttaaaactttttaattttattctgacaacagctaaaactatttaatcttctgtttcgctcatttcttccaaggtaaacttcagttggttttacgtgatttgctatttcttcttctttgcatttacaaatgatctgtgatcatattactgatctttgtaaagggctaatatctacctgcaacatttggatatgacagtatttaccctttgtaaatacacattttctatttatcttcaaaaattaccattcattagtctgtgttaatgtctgtttactattgtgtcattatgaatgtgatgtgaacatacgaagttgaacttatttaaacgaacactctcatgagcttctaatccacattccttccttttccttctaagttaccatttcttaaaaatcttttagaagtttccttgatagggaaaacacaaattattgaggaatttttctttctcttgacatctgtttatagttactctcttgttccagcagtggatatttcccctccatgtttttctttgtctaaacatatgttcaaaacaaaacacttttattcttctttgcaggttttacaaggatcaacttttagttttgaaacctgctattacttttagaggccattttttttttctctaataatgtgagttcatgcgggctgaagtaattggaatactttatagaaaagattgaatttgtcttctctctgaactctagtttgaatttctaaattttatgaatcatctagatattaaagaggaggggcatatcaaagaggagaaccctagcagagataagaggcaagagtaaatgtttcatgtatgggtaagagtggatttgtatttacctaagtaaaggtagaccctggacaataaggttggatagatgtggaggtggcaaaccatggagggtcttgtaggtcaagtggatgtttttagacttgaagtgttaaattattatctgaaatcattaagagtctttttagatccttgagcttcttgagaagaccatggatattatgcagttattatataatgttttaaaatagtaagtattttagtttaactgtcttatgtaattccatataaatggatgcatgttctttaaaaatgttaatgtatttcagtaaatcaaaatatactttttgactcatcatttaaaggaggccttcagtgaatgctctgtagaggattattttataatactaattttgatatcctaatttatttgttataaagtttagaaggtttgaagaatttaaaatatagtgttaataaacacactgaacttttctttttttatcttgtatttttatatagtacaacagaaaaaagatgaaatgtgaatagtaaagagtctgtgattgttgttcatagggcccaccaagctcttcctcgagattctgttgtagaagatttacatttacaaaatagatacctccaagaaaaacttcatgctttagaaaaacagttttcaaaggatacatattctaagccttcagtaagtgtatatcttttattatttttttcttttttccatgttaaaatgcatgaaagtgaaatcaacttctttcttaatctggccaaaagcattacatctttctcattaatagtaatacagtaaattcaacttttatttttaacaggtagtgatgtgtaataatttatttaatcctttttaacataataacagtaaacttaagattcttaagcttttcataaagctcataaatgatttctagaaattttaaatatgtagttatcattatgtattttgctgtagcagcagtatacagttaaataaaataggaaaacatgttccaagactgttttcattcaaatatttatgctatatttttagcttataaaaactcattaatcattaatgtaaaattatttgttggattttttaaatatttagtgtattatttttgtttcttttttctttccatgtttcttcattcttccaccttaagcagaatcaggtgtgtgacacaactatgttttctatccttgttaccattattaataaatacaagggcatgatatttttcacaaaagaaacactttgttcagaaccaaaaaagatcatggcaacagtcagaattaaaaatggtaaaagactaggtgccaaagatgacttacataattgggtacctagaaatattctatggtattacagtaatgatgaaaaatacaaattagaacacattttagatcctattgagttaaataaatcagagtcaagaccaaacaataaataaagtcaatttacgtcaacaaatggtaagttggcagattttaactccctttttgaaaatgaaccatgatcctaaggttggtaaaattaatcaagaatgttgtcaaaatgataaagataaaaatgaggaagagaataagataggcaagagtgagaaaggaaagagacacatagctgaaaatgtgagtcacaacaactacatagatccgtagaatctgctatggaggactgtgattatgtgacagttgctgatgccgtggcttagtgagctgagggtgatgcacaggcaggcgatgtaactgatgcgtcagtccagccaagaaaggacgcgtccctggtttggctacgtggccgtcctttatttctttgttaactgaattttcttatagtaagtagcttacgtacatatatagtgcaaatgggaaagtgtgtaagatttagaaaaagcattaactattagtaaactttatcttaagctctaacttttgattagttcctacaaaaattagtgaatatgcattttctaatttagtgctttttttttttttacaattggtgttcacttaatgttatattagataaatgaatagcaaaaataaggtactttagagttgattgttttgccttacaaacttctaatccatccagctgtatttagaagtaagatctcactacagcgaattatatcagtaaaattttgttacagtgttgtgcagtgtcctaagatgtatactaagttccttcagtggctttttttgccatgttttataacagataattttgttataatgagaaaaggaaacttggatgtgttgctgtctatattgtgttaggctcaggcaggatgctgtggcttactcatttaatcactttgggaggcaggggcaggaagattgcttgaggccaagagtttgagatcagcttgggcagcatagccagaccctgtctctacaaaaaatttagacagatgtggtggaacacatttgtagtcctagctattagggaggctgtggtgggaggatcatttgagcccaggagtttgatgttacattgccctattgcactccagactgggcaacagagtgagacctgtctctaaaataataataatgataatgataaatggtgttaggctctgtgcctaagtatatttttcacataggctgggtaaagtggctcatgcctgcaatcccagcactttgggaggccaaggcagcaggagcatttgaggccaggagtcaaagaccagccttgagagaccccatctctaccagaaaaaaaaaaaaaaagaaacaattagctgggtgtgattgtgcacacctgtagtcctagctactcgggaggcagaggtgggcagatcacttgagcccaggagtttgaggttatagtgagctaagattgtgccactgcactccagactgggcaacagagcaagactgtctcaaacaaaaacaaacaaacaaaaagcactttgcagaatatcagtctaactctacagtttatggactttttatgtacgtactacttttggctagcttacattgagatacagaataaaagtttgttcatagcatttatcgtttttttctttatactgtccacctgagatattccagtcacctaagtcatggaaacatcaactaaaattaaatatctatgttaagagaaaatggctgaaagtgatttaattcataacactttttttcacatgctaataaataagagtttgagacttccactaggcattatctctaactcctatccactaagaatttgattttaagtagttgatggcttttaaccggattattcttctgtaagagtttggaagtctcgtgaagttcgttatacaagaattctgtttacaagagagcattacattagaatttgtttttcagaaatttggactatctcaacgaatacctttagttttattatttcaaaatgcaagggaaaaaatgagccataatcactaatagtaactgcatcatattttagtgagaaatgtgttaaaaatatcctcatgtgagatcttccttagatagaattaccctctactctaatatttaatatattttatatctaccaatcagtgatattaataggtgtttatcatttgctgaatcaaataggtacaacagaagacaggaagtttgggagatagaagagctcagggacaggaaatcacagatgtccatatctgaaataaccttaaaagttatcctgtctaatgccttcacttataaactgtagtggtagaatttgcctagtattaacctaatagtggtagatttgaatgtatacttgggctttcttattaagtggaaatgtattcctgtgatttacatatatcaacaaaaatgtttgtcttcttttttttgctacgacatatgtgcatgtgcacacacatctcctcaaacaaaaatcagatggacacatgcagtcattggatctaaaagatgttataaagttgtgtataataggtattttataataatatattttaagacccataatgtcggtggagtaactgactttacagcccatcaagccaatagagagagaaaggagaaaaaaatgaaagttgtgctgaataattaaaaaaaattatttcctatgatgcttataacagtcctatgaggtaggtggtattctaatttatagaaaaaatgcatagaaaaatataattaagcacagttaaaaaaaataaagtttagaatgagaagtaacaacataaataatgacccaatgtagattcaggtcaaaagaaatgaaaatataatattaatggttttcaaagagggaaccattactttagctcaaagaatgaaggagggctttccgaaggagtaaagaattatggcagttcttttgtagcctagtgtattcatttgctaaggtggctgtaacagactactacagatttggtggcttaaacaatagaaatttatggtcttagttctggagacctagaagtccaaaatcaagacatcagcagggttgatttcctctgcacaatcagagggaaagatctttcccaatcctctctccttggcttataaatgtccatgttttccctgtttctttttatcatcttccttctgtacatgtctctgtgtctaaatccccaaattttctcttttcataaggataccagtcacagtcgaatagggtttaccctgaaatctcattttaacttgaatacctctgtaaagacccagtctccaaataaagtcacattctgaggtactggaaattatgactttaatatataaatgtggagggtaaggggaacacagttcaacccataacggttagataacaatcgtgctttattttggactagtaaaaccaccatagatcagtttaaccattatgaaattatacatgaaggcattatatgtatggacattattaagtcatacttgctttgcttccattgtaattaaaacaaaccatactacctttgttctgcaagttttgtattctaacttatttatttttggctttcaccagaacactccgattttctcatattcctttgaggaaaaaaagttaccttttgacagtattttcttatccagtatgtcttttatggcttttatttattaaactttaaaaatattcctaatttcatttccctgaagatttcaggaatagagtcagatgatcattgtcagagagaacaggagcttcagaaggaaaacttgaagttgtcatctgaaaatattgaactgaaatttcagcttgaacaagcaaataaagatttgccaagattaaaggtgaatttaatgttttttattaggaaatctaatgcctaaaactccttccttagttgttatgtttacttttattagcttattaagaagtcaaaaatgcatattcctaatatatcatggtgatggtatactttatacatttgctctttagcatttatttgttgaaggcctactttatattaaacactcctccagatgctgggaaacagcagtcaaaaaattccttatactcataggacttacgttctagtggagaagactgacaataaacaagtcactaaatagtatgtcatctgatgttagtgctaaggagagaaataaagcatgattggtgtaaagagtatggggagagagaaggggtgtaactgaaaatagagtagtaagggaggtcttccttaataagatgatatatgaacagagagctaaggaggggtaaaggaagtgagtcatacagatactagaaaaataattacagacaacagaaatagcaagttcagatgtcctaaggtgggaggatgcgtggtatatttcattaaaaattatcacactgtaaaatataagaataatttgtttcttttagaaattttactttattctgatattaataatgattttttaatctttggttttccaagtcttaccctatttatgggaatcttttttttcttttggctagctaattgcttcagttttgttttctaatctagaatgttagcaatctgttaattccactggtaatgatatagttaagctatgtcttgcttctcacactttatttatttatttactcagggcactaatctgccattttttcgcactttttttccttttttttttttttggtactgcttcttattctggtttttacattgatagaaccaatgttagacgttcatttgccttttgctgtgtatatttgggtaaggatctatatgtgcaatatatgggacagttaaaatcagaattctaaatttgtattattgcatcaggcaataatgtgggaaataccttgacatttcatatacacaatattcttgtattaatttaacgtcttagttcaaaatcttccttgttaatatagagaccctattatttggtttggcaatacagttgaagagattgatggttcttatgaattgtttgccttttcttttcaatggctgtagctatgttaaattattacatgtttgcttgttatctttcagaatcaagtcagagatttgaaggaaatgtgtgaatttcttaagaaagaaaaagcagaagttcagcggaaacttggccatgttagaggggtatgtgagaatttaccatacatttgttttggtttcagcagtgataagccagaaatgaaaagtttagatatgttgtaaaagtactgatatgcctctacaagtgccctgtagtttcagtgtttattctgcatctgtaatataaaacagtaagcatttctatgtgtctcaaagtattttatcatctgttataccttacatactttcatctctctttttattgaatatgcctccataccttgaaaacatttaacttccaggaatccttttgtttatggaggtaactgctaactggtccttggtccaatgctgccattttgtaaccatttgttatgatatcttcccagcttggtataatgttttataattacattgttcctccccctctttttttgtgttcttgtaattttctccctatgttattttgtattcattttatataatgaataaatgttgcttatgaggtcaaggccaaagacttaagctcctgttgatttcatgttgctgagtgtcataaatggaagcaatcataatgcagagtcattctggtagtaatattaaatatatgatggattcagtgaaaatattatgtgttattagaaaaatattcagaacaggccgggggcagtggctcacacctgtaatcccagcaatttgggaggccgaggcgggcagatcactggaagtcaggagttcaagaccagcctggccgacatggtgaaaccccatctctactaaaaatatgaaaattagctgggcatggtggctcatgcctgtaatcctagctactcaggaggttgaggcaggagaattgcttgaacctggcaggcggaggttacagtgagccatggtcacacaactgtactccagcctgggcgacagagcgagactccatcttttaaaacaaaaaaaaaaaaggaaaaatattcagaacagtatcttgctggcagcaacatttgtttcatcaatgaaaatatgtgttaatttgaccttttctatctaagttaattatgaaagtgcatactaaaatgatgtaaaagtttatatttcaggattattcttattcatggatgattaactaaaatgcaaaaagaaattaagcatactgtttggctaaactgttaaaaattatttttattttaaatgataagcagttaaacttattaagtgatgactcatctctgctgatatatttatgcaaggttttttatttcagataactcttctatttatattaaacagaaactgtatttctaagcaatagcatttcttagagaaaattgcctctattatgttgcaattaaaatttaattactcatgagctctttaaagacacaatttctcttgtgtggttttatttcatataaagaaaaactctgatatactggagagaacattagctaaatagactatttagacttaatcattttgatcagacatcaaggctagactatttaagctgttacttattagctgcatgattttaggaatgtcaaatttcctaagtcttggttttcttgtatttaaaatggaaattataattcctatctcatagaattgttttaaggatgaattgaattaatacagttttgacttcaaatattaggaattattgagtataataagcctgttgtattgttggtacttcgtattatacttactaaaatatttgattaaagatttaacatattctttcgtagtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggtaattttttttaatgtgatcatttttaggggaatattttacgttttgttactatttaggaaaatttcaaatatgctcattactatataaaatggctttaatgaatacaatacatattttataaatatagaaaaaaacttatgagaggcaaggctaagggttatagagtaggtctacctgatctttcttgttatttcaagaccaatacttttcacttttctctctgacagcatagattaattacctgtgtctctcttttttttttcttttgagatggagtactgctttgtcacccaggctggaatgcagtggtgcaatcttgactcactgcaagctctgcctcccgggttcatgccattctcctgcctcagcctcccccagtagctgggactacaggtgcccaccaccacgcctggctaacttttcgtatttttagtagagatggggtttcaccatgttaaccaggactgtctcgatctcctgacctcgtgatccgcccactgcggcctctgtgtctctttgtgaaaatacagatgcccaagctcccatccctgaaattgatttaattattttagggtgggtcctgacacagatatgtatgttgttgttattttaagtcatcaatttattctaatatgtagccaacgttgggaacttcgttctcactaatattcaaatgaagactttaattctaatcatatcaaatatggtttctaaaactactttgaagatttatgagtttataagattatcttttatttccttgttttgataatgtatactttttattttgtttgtttttttactaggctgaattagaaaaacttaaagctcatcttgggcatcagttgagcatgcactatgaatccaagaccaaaggcacagaaaaaattattgctgaaaatgaaaggcttcgtaaagaacttaaaaaagtatgacttttatgactgattataacttttgatttttattttacttaatacctcttggaaaaactggaagtagatccttgatgagagtgtctgtaaaggtagatattaagagattgaggaattgtgtttctatgcctgctgtcatcacattccaccatgaaaaacattgataataaaagttaatacatttaggctgggcacggtggctcacgcctgtaatcccagcactttgggaggccaaggcgggtggatcacgaggtcaggagatcgagaccatcctggctaacacggtgaaaccccgtctctactaaaaatacaaaaaattagccgggcgtggtggcgggcgcctgtagtcccagctactcgggaagctgaggcaggagaatcgcttgaacccgggaggcagaggttgcagtgagccgagatcgcaccactacactccagcctgggcaacagagcgagactccatctcaaacaaacaaaaaaaagaaatgatctacgttgcttacacataccttatgcttatagctaggtctcgtaagcattaggaagtcaaaacaaagaatcttttacatgtgtaaaggtataaactatcccatttttctaaaaatatagaggaacaaagtgtcaaatttaaagtaatcactagtaactaaatatattcctctgacctcattttcgtgatctgttgttctaattattattggccatattgctgctttaaaggagagatgttgaatttgttgaaattttaatcagcatttagagccccaggttatttttgttttccaatttgtaatgataattttgaatacactgaatctatgagaacagtattatgttttctcataaaatactaattagcatttaatgataggaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaaggtaggaacagagttttaaacttgtacaaagtttaatcatttcaaattttggcattgttttaaaagacaacactattctggataacctggtttcttcctgatgaacagtttgtttggttgttgttttaacataatactttttttctgttgtagtattgttggagactttttcttccttgaaatgtttaacttgtttaaccttgtttgggtggcagggcatggaacagtgtagagctggggctgggcgaaggagttggagctgtgtgtgcgtcatgaagctgtcatcagctatgagcctgggctgaggctgctcagcttctcctgggtgctatttttctccaactgcagcttcagcttcttgattgtataatttgcttcctcaagtatgagccaggaataattgagctgtcttgtcacaatgtgtggcatactggatctaggctgtgctgcaatgcttttagagttatatcctgggcaactttctcttcagatagccccaagagatgaattcagcaccagctttgatgttttactagcttctgctttctggtacttgattttctcccaccccgaacacatgggattccaacctgtgaaactaatttttgtggctatgaaagaggtagtggtagtttatgagtaaacattcagtctgttgccactatcatcatgtgtggttcatcatgactgtgatgagtaggtaaaaggctctttgtgtcattctcatttccaattttaagcagctgcttcaaggagtctggaagtcattgaccagtgggatcctgcctgtgtcttttcccattaaagccatcctgtatgaagtggtatcctttaccatctagcacatctgccgcccccatttcaaaaggcatactcatctttatctcaacattctcatacagttccttatgtccatgcacctccaatgtcccctttgatgtctttgaggttttcatcttccatgtctgctatttggaatggtcttgatgggaggcaagatagtgatcactacaactaggatgggagtcttagtaccgtgaggctacagcaagtcccacagagggcctgctgcactgtacttgcctctgtcaaccaagtctaaggagaaagattaagcaggcatattaaaggacagcccagatggacatgaagtcctggaggaggccttggttcctgtcctaatactaaacctagagtacccagaatccacacttctccactctagctctcacttttcccatctacacactgggaaaaattattctgtcagaaagccagtgtcaaggtgagaacaaataacaaatgtgatgatatggagtgggagaaggggtctcttctactgtcttattggaccctagcagtggctctgagccagcagtcctgtcagttgatttcttggtcgttcctttgttttcttctataatcacatgtggactcagaatgaattttgagttactctgaaatctatttattcaacagatatttacttagtacctcctattgccagactctgctttatgttggatattattttttaaaagcccaccttgcctagatttcctcaaaggaccaggtggcttccctggttttgaaagaccctaattcttactatgatcttaagtaaattatatcctttctgtgggctcaagttctttctaagagggctctttggggctacaaaagaaattgttagtgcaaaaagagtttataaggtttataaatggttagtagaggtgatgatgatatttaaccataattgaagatgactttgcattttagatcatatacgtgtttttcgtctgagaacgatacaggtcactgagcataccataagccttcagtaaatcatttgcagaagacattgcagaagacataagtctaagtagaaatctcttgacagagagaaggctcgttttgatccttgacctcaaatttaggttccctaaatccattaaaaaagagaaagaaaaagaaaaaaagttactaaagtttaaatctgggaggattatatacccttctcaataaagcagtttagagagatctcttttgggacccatgacacaggtcttgctcatgctgacatctttatagttgctttattatttattcaacaaacttagtaacacgtattctatgtcaggccttttcctgactactgggacaaaccagggtgatgtgggggctgttttagatagggtgatcagaggaggcctctctgtttgggtggcttttgaatagaaaattagatgaagtgaaggagtaagcttctgatatttcactgtttacttgtggtagatctgtgataatctctgtcaggttaaaaacattcccttctaatctaagtttctaagatctatcaaaagctgtttgaatatatttagacaatcataattttcctttcttgtattatcctagcagattttgttgccaaagctatactggccattttaacttagaatgcagtctttctattcatttctctggaaaagtttggatattgtaagcattatttttctttaggtatgatgaacctgcagaactgtttggttcaattatgaattttttttttctggagtctgtatttttttgaactattaatcatttctttaatgattataaatctattcagatttttacaagctttatccctctcccatcatacactatttttcttacccatgcttttgcacaattttttcctctcccttagtgttttcctacctagatacctcctatgtgtgtctgtgtatgtgagaaaagctttttatttgccatctttatatttctaagaatatctagtaatacagaattttatattctgaagaattttactttgcattttcttattttgtgattgaaaaaaggtattaattttaaaatggtcaaatcaggctccatccttggaaaatacccaaatcctttattttgattgggccatctgttaattagggataccttatctcttgccaccactttttaatgctaaataaatatgtagctaaaactttgactagaagaaacagtaaaataagatattcttgcttatttttagtacagttatttgaactgacttttaaatcagtgacataaattatttgccatgtctatactttttttccttatacttttagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacaggtaagtaacgtaatttttctttacatgataaaataatgcataatatcgcaagatgttccttgcattgtcttatatagataaaaatggactctattaagaagacccatctaactgaagggcaccccattcacccatttgcttaagccagaaactttggatcatcaacgacttcattcttttcattctccacattttctatcattaaatcatgtcagctctattttcaaactatatcctaaatatgaccacttcttggtatcttgagacatcactaccagtcttgtccaagctattgttttatacctgaataactgcaataatttccaagctggtatctcagcttccactcttggattatttcaccctatttctatttctgggctgtctccacacagttgccaggtaacccttttaaaacataaagcacatcacaaagcacaaagtcctatcctcagaatcttccagtggttctccatcaccctaaaataaaacttaaaagttcttttcatatcccaaaacaacatatgaggtctggcacccagttttcttcccaatctcatcttctactacttttcccttcatttcattcacaatgttttaaccacagtaaccttctttcagtactttaaacaatccaaactcgtttaagcgtcaagtccttatacttgtttcctttgtttagaatactgttcacccaaatattctcatagcttgctcccagacttcatgtctctgctgaaatagaggctccttagagagaccttccctaaccctaaccctaaccctatactacttgccatcactctttatcctcttaccctggattattttttcttgatagctcttcctaccatctggcactatattacatcatatcatattaaacacacattctttgtgcttccccactaaacaaggaccatgcaagatggaacattgccattttgttcactgctgttagcctctgtgcctaggacaatgccagttatgcagtagttactcaatacttgttgaatgaatggtgaatagaacatagaaatttgcctatgcgtgcttttgaaaaccatattttaatattacgctttgttaaaaatgtgtatctttataaatcctcatatttccatggcaaaccttatcttctaacttttcattgtcctcaaagattaagattcttaaacatgttcctgaaggtgctgagacagagcaaggccttaaacgggagcttcaagttcttaggtacatcatgtattcatatgactactttgtttttttctttaaaaaaaaaattattagtttttatatactccgaattgctacaactagagacaagcatttttcgactttactgcctaacaggcttattaggtccttatttcttccctctaatgctaatcactctttttcataatacacactagaaaaaaaggataaacccaactctaagtttccagtttgtaatttagtttaaacttttctaagagcatagaatgagttaaaccttagcttcccagaggaaaatactaatgaaagagaacaagtaatttttttactttcaggggtctctgtagcctgctttcattaagctcctcttataacgaaaccacacttgcaaatgccatcaggtcagatattaagaaaaacgtgaaggcttttgtattccaggctttttgtttgagaatggtgacattgtagcattgagagtaaatgtttacttcgataaaggctagcttgttctgattactgtacatcactagttcataagaaatgcccatatattttatgaagcaatatctgctttatttttttaacacattatcattgtgttctagattagctaatcatcagctggataaagagaaagcagaattaatccatcagatagaagctaacaaggaccaaagtggagctgaaagcaccatacctggtaatgtattttaaaaaacatgttagctacccccaagtttttgaatttgggtttgccttttttttttttttttggctcagatttctgatcattgtctccctgtaaaatcgaattcctgataagctttgggtcttttgtctctctgtgctattaatataaaaatattcccatttttctctttgtgttgtttatactatagagtagcaagtacccaagtgttcttctctttgttctccatctgggtgttacagatttaatcacaatacagtgctaagcaatgaatactaaatctgttgcttccagtttctaagtataggctctttcaagtcctctgaacatttttaaaaactgcaaataagtaaatactgcctatatttttttccgtttacaaagtaaaaagaaaatctttctgctcccttccattcccattcaaaagtgattactaatcattcctcattcctgcatatacatacacacatattttgtatacatatatatcacacatatgcatacatgtgtttgtatgttcatatgtacaatgtacatatcctcattatttgtggattctgtattttctaaatcacctcctcactaaagtgtgtatgtaatcccaaatcaacactcgcagcacatttgcaaacatccacagagccttggaaagtttgaataatccaacctacatgtccccagcagaagtccaacaaggcagtgctcagtatcctcatttcagttttcatagagaaatgagcagaggatggagacagtagagggcagcacagcatagtgcaagaagctgtggctctggggcctggtggaagggatttgaatcccaattctgaggcttgttactgctctagccttaggagagtcatgtaacacttctgaatcttgttttcttatgtaaataaatagaatttaccaggatgagttatctttaggatttaagattatcatctgtgtgagatatgtaggtgtatgtatatatatgcgtgtatgtatatatatgcgtgtatgtatatatatgcatgtctgtacatatttcccgtagcagcagtggtttgatattcactaattgggctaactttatagaccaaaactactatggatagagaatactttgtttgcatttacgtatatatattttcttggcaagtaacataaaattgaactaatactatacacatttctagcatatttgcctttaacagtttatcatggacatcttttgaggtctgttcataaattatctcatccatttaataattccatagtgtattattgcatgtataagcacatcgaaccatttatgttttgatggatatttagtttgcttccaagtttctgcttctataaaatatgattaatctattgacctaattatgccattgtgataggatgatagagatgccattctctccaaaggattataccaatttatatctgaactatctttgactatctcttgtagctttttcagtatgctatgtagtcctattactaatttgtaataaaagccatcatgtgtgagttgtactagacactatgctaattgccttacaagcattctatatttacaaccatatatgataggtattactgtctccattttatgtgataaacaaattcaaagtggttaagtaaccattccctaagccagctaggaaatagaggcaggattaaaatctaaatgtatgaaactccacagctccttggcattcctagtccttaacccgctatgctatgctacgtcttggtaactaaaagtacatattaaatactctcaaaatatgtctcatagcagccagcttggtatgtacactagacacagtattaatgctgttgatgtgaggaaaattttataattttccttccatccatatactaaccaggcccaacagtgcttagcttctgagatcagagatcaggtgcatgtgcattaagggtcatatggccatagatagttctctaatctttccattcctcagtttcttaagggaatttctgaaccctcaaaattccttatttcctaagtagacagattacctgtcatttttcaaagattaaggcttaagatcaaaccagaactgttttggaaattctaaatcactgtctatataaatggcaagataacttttaagatatttataccaagcccagtacagtagcacaccacacctgtaatcccagcactttgggaggctgaagtgggtggatcacatgaggtcaggagttcgagaccactctggccaacatggtgaaaccctgtctctactaaaaatataaaaattagccaggcatggtggcacttgcctgttatcccagctacaagggaggctaaggcaggagaatcgctttaacctgggaggcagtggttgttgcagtgagccaagattgcaccactgcactctagcctgggcgacagagtgagactgtctcaaaaaaaaaaaaaaaaaaaaagatacttgtcccagccatgaaaatgtttgctgccccttactttcgcaaacttttagtattttattatttttcaatggctgtaaaatatgacttattaaatgtagtataatataaagaaaagagatatctagcaaagatagcattaaagcaaaaatcctatttgcctgctgataaagttagaggtgttaacttggagggtgaatccaataaattagaacttttgtgctatatttggagacttttgttttcctaccaaagtatcagggctatgtcttacttatctttgtattacacagcctgcatgacacgttttgcacatagtaattgcacagtaaatgtgtaataacctacatggaatagccagtgttgtgttggatagcgggagcatttggctagcttatggttatagtcccttacccaacagtctgcttttcttctgttgtacttttagtacctaacaagtttccctggctttaggattttttccatgtaaaatttctatcatgtgaagaaaaaataacttggcctacacttctaatacctagcacatacctctttctgcctgctatgaaattataatacttgatggagggaggcagcattaagtgtttacatcctgaagtatttcagccataacatccagtgttttccaggttctaggtttcataaaatgtatctctgttctctagaacaaatccattaccttgaactcattcgtagtgggaaaaagctgagtctaatttgtatgactttttcaacagatgctgatcaactaaaggaaaaaataaaagatctagagacacagctcaaaatgtcagatctagaaaagcagcatttgaaggtaatatttaattatattttagtatcgttttgtgaaaacagctgttgaaaactattttcattaccatctttaactacgtatcctaaaaaattcagtaataacatcttatatttgacctttatattgcaaagttaattatgttcatctgactattcctaacatattagagttaacaaaaaattcagactcaacataggattaagtagtaaatttattttttaattgtaacaaatatatgccattagtatgttcttaagttttgggtcacattggcaacagtgtctttattttttttttgaaattcttttcaggaatcctaaggttatagttcccttaaaaaaatatttgctgttttacctcttttaagactgtaaacaggacaaaaaggcatggatatgagaattagctagtgatcactggctattctaaatagtcactaaggcttgaattgtctcttcaccagatgcctgtcagaagtcccaaaggtttccctgatcatattaataactttataaaaaattgatcattattcattaaatattagatattagtaaggaaaatataaatgaagtctaaaccaaaactcttaaccagactaacttcaatgttatgaatcacaaaatctttttgattgattgctctattgacaagctcttatatgcttttagagaaagattaagtcccattataagagatgataaattttagtcaaagactagaacacaacttacagaatacataactggacttgacagttaacaacttagttatttacactgtacaatggaacaaagaaaaatcttaattcttctgcctttattgctgtatttgaccattcaggaatactttggctttcatatttacaattaaatctccttgttcaaacgtaaaatatgtatatttcctatatgcaacttttaaagataatgtttccattaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttactaaaggtcacctataaactttgtttcatttaactatttattaactttataagttaaatatacttggaaataagcagttctccgaactgtagtatttccttctcactaccttgtacctttatacttagattggaattcttaataaataaaattatatgaaattttcaacttattataaaagtatatacttgaagacttgttacagccatcaaataggcagtgagctctggtgaatttctctacattcataatgctttaagttttttaaagaatatgagggtaaattcagcaattttaagatttaaaattggagtttatatatacttatttatacatgtgttaagattctcaaatcctatgatatatacacagatgtagacatacacacatataggttatgaggttgagccgtatgttttgttaaatatctagcaaaattaaattgagcttaaactatttaattttgaaaattagtgacattaaattgttgtctttaaattaccactcagttatagtgaagagaatccagcctttgtccaagcttaggagcttactattacttatgtttttatggctagagattaaaattaatttgctggtttttaataaaattttgaataggtacttgaaactggaaaattcatttcttcacaaatggtagaaaacatattgatgtttcataatgaacagaaaccttgagatatttttaaatatggtagaatattcaagattagaatataatgataaactaattttatttgcattttatacatcaaatattatatctttgagtctaacaaatttctgattatctatttttaaaaaatgattaaacaaacacttaatatcaaaggcagagtcacatttgttcaggttcatgttgataataactggtcttgaattcatgtaagtatctggaattggccagcataagccaagatgatggcgatggacctggaaagagaaaaaaggtattagctttagcatacagatactatcttcattcacttagaaatatttgagtgcccactgtctgccaggtactgggtaagactccactttatcttactgtttttttttttatagctggtcttctcctgacatattacatatttgtttattgtctatcttccccattagaatgtgagcttcatgaaggcagacaccttgttttgttgactgtgtatgtccagcacctggaaacatgccaggaacattatcaggggctccataaaatgttatgaatcatagagcctgttgctgagtacagactagctacctgaaaaatgtgggcaaggaggcattctaattgaaaggaatgttatatgatagaatggagttttttgctctaattgcaatcatgtaaatatggttacagcaaacacagtgtgagtgtgtttgcgggggcagaggtggagagcagttctgggaaattaagtaggggccagatcatgaatgtttaatgtaactgaacagatttatgcaggaaagtaatatgctcattcatttgtgaagaatggagaggacagggctaaaactgaaagatactatttttggagggtgatatgatagttaaagcatgaatcttggcttaaattaaaattaacagttgggaagaagtacatagattagaactgttaggaattgataactggatgtatgaagtaatgaaaagaggggttaaaattaatatccaagtttttacttcagtgaattatgtggactcagagagttacaactaggcccagttttgaatattatgaatttcaggtacctgtgagaaatcaaacagacaagtccaggagacagaaagggaacaaagtataagataattcattctgtatatatcaactgctaccttggcactctttttactcaacagattctacactcaagtctgggagatgctgagttaagttagtacaagatgtctctgagagcactactgtgcaacatgaatttccacgcaggggatatatagttatcactttaattctgaagcacacctgttatgatcgatgccccacacaatacacactgggaagcactgttttacctcttaaaaaatggtggtacatgggatgagaaaattaaatttctttactgcaaataagaattgctgccatttgtagagagaattttagtgtcacttctactctattgtaggaatataag

(19) United States
(12) Patent Application Publication
(10) Pub. No.: US 2024/0318186 A1
(43) Pub. Date: Sep. 26, 2024

(54) TRANS-SPLICING MOLECULES

(71) Applicants: Ascidian Therapeutics, Inc, Boston, MA (US); The Trustees of the University of Pennsylvania, Philadelphia, PA (US)

(72) Inventors: Philip R. JOHNSON, Bryn Mawr, PA (US); Bruce C. Schnepp, Havertown, PA (US); Jean Bennett, Bryn Mawr, PA (US); Scott J. Dooley, Clementon, NJ (US); Krishna Jawaharlal Fisher, Durham, NC (US); Junwei Sun, Philadelphia, PA (US)

(21) Appl. No.: 18/673,713

(22) Filed: May 24, 2024

Related U.S. Application Data
(63) Continuation of application No. 17/047,496, filed on Oct. 14, 2020, now Pat. No. 11,993,776.
(60) Provisional application No. 62/658,658, filed on Apr. 17, 2018.
(60) Provisional application No. 62/658,667, filed on Apr. 17, 2018.

(51) Int. Cl. C12N 15/113; C07K 14/705

(52) U.S. Cl. CPC C12N 15/1138; C07K 14/705; C07K 2319/71; C07K 2319/85; C12N 2320/33; C12N 2320/34

(57) ABSTRACT

The present invention features nucleic acid trans-splicing molecules (e.g., pre-mRNA trans-splicing molecules (RTMs)) capable of correcting one or more mutations in the ABCA4 gene or the CEP290 gene. Such molecules are useful in the treatment of disorders associated with mutations in ABCA4, such as Stargardt Disease (e.g., Stargardt Disease 1) and disorders associated with a mutation in CEP290, such as Leber congenital amourosis 10 (LCA 10). Also provided by the invention described herein are methods of using the nucleic acid trans-splicing molecules for correcting mutations in ABCA4 and CEP290 and for treating disorders associated with mutations in ABCA4 and CEP290, such as Stargardt Disease and LCA 10.

### CROSS-REFERENCE TO RELATED APPLICATIONS

The instant application claims the benefit of priority to U.S. provisional application Ser. Nos. 62/658,658 and 62/658,667, both of which were filed on Apr. 17, 2018, the contents of both of which are herein incorporated by reference in their entirety.

### SEQUENCE LISTING

The instant application contains a Sequence Listing which has been submitted electronically in ASCII format and is hereby incorporated by reference in its entirety. Said ASCII copy, created on Mar. 17, 2019, is named 51219-016WO2_Sequence_Listing_04.16.19_ST25 and is 608,489 bytes in size.

### FIELD OF THE INVENTION

In general, the invention features ABCA4 and CEP290 trans-splicing molecules.

### BACKGROUND

Stargardt Disease is a progressive ocular disease characterized by loss of central and color vision, which can occur rapidly or over the course of multiple years. Peripheral vision generally remains intact. Various mutations along the length of the ABCA4 gene can cause Stargardt Disease. Treatments currently in development for Stargardt Disease include lentiviral delivery of ABCA4, chemically modified variants of vitamin A, and retinal pigment epithelial cell therapy.

Leber congenital amourosis 10 (LCA 10) is a condition characterized by severe visual impairment beginning in infancy. The loss of vision is associated with photoreceptor death due to ciliary defects. The most common known mutation associated with LCA 10 is a point mutation in which an adenine is replaced with a guanine at nucleotide 1,655 of intron 26 of the CEP290 gene, which results in a splice defect in which a cryptic stop codon is spliced between exons 26 and 27. This autosomal recessive mutation causes the production of a nonfunctional centrosomal protein, causing the blindness characteristic of LCA 10.

Adeno-associated viral (AAV) vector-mediated gene therapy has a proven safety profile in humans and represents a promising approach for treating a variety of genetic defects. However, AAV vectors can have limitations dictated by viral biology, such as packaging size constraints that can hinder delivery of large nucleic acid molecules, such as those necessary to replace the ABCA4 gene or the CEP290 gene. Thus, there is a need in the field for compositions and methods for correcting mutations in ABCA4 and CEP290.

### SUMMARY

The present invention relates to nucleic acid trans-splicing molecules, and methods of use thereof for correcting mutations in the ABCA4 gene or the CEP290 gene. The compositions and methods provided herein can be useful in the treatment or prevention of diseases associated with mutations in ABCA4, such as Stargardt Disease (e.g., Stargardt Disease 1) or mutations in CEP290, such as LCA (e.g., LCA10).

### ABCA4

In a first aspect, the invention features ABCA4 trans-splicing molecules. For example, the invention provides a nucleic acid trans-splicing molecule comprising, operatively linked in either a 3′-to-5′ direction or a 5′-to-3′ direction: (a) a binding domain configured to bind a target ABCA4 intron selected from the group consisting of introns 19, 22, 23, or 24; (b) a splicing domain configured to mediate trans-splicing; and (c) a coding domain comprising a functional ABCA4 exon; wherein the nucleic acid trans-splicing molecule is configured to trans-splice the coding domain to an endogenous ABCA4 exon adjacent to the target ABCA4 intron, thereby replacing the endogenous ABCA4 exon with the functional ABCA4 exon and correcting a mutation in ABCA4.

In some embodiments, the binding domain binds to the target ABCA4 intron 3′ to the mutation, and wherein the mutation is in any one of ABCA4 exons 1-24 or introns 1-24. In some embodiments, the target ABCA4 intron is intron 19, the mutation is in any one of ABCA4 exons 1-19 or introns 1-19, and the coding domain comprises ABCA4 exons 1-19. In some embodiments, the binding domain is configured to bind intron 19 at a binding site comprising any one or more of nucleotides 990 to 2,174 of SEQ ID NO: 25 (e.g., any one or more of nucleotides 1,670 to 2,174 of SEQ ID NO: 25, e.g., any one or more of nucleotides 1,810 to 2,000 of SEQ ID NO: 25, e.g., any one or more of nucleotides 1,870 to 2,000 of SEQ ID NO: 25, e.g., any one or more of nucleotides 1,920 to 2,000 of SEQ ID NO: 25.

In some embodiments, the target ABCA4 intron is intron 23, the mutation is in any one of ABCA4 exons 1-23 or introns 1-23, and/or the coding domain comprises ABCA4 exons 1-23. In some embodiments, the binding domain is configured to bind intron 23 at a binding site comprising any one or more of nucleotides 80 to 570 or nucleotides 720 to 1,081 of SEQ ID NO: 29.

In some embodiments, the binding domain is configured to bind ABCA4 intron 23 at a binding site comprising any one or more of nucleotides 261 to 410 of SEQ ID NO: 29 (e.g., from 1 to 200, from 6 to 150, from 12 to 100, or from 20 to 80 nucleotides of a binding site within or encompassing nucleotides 261 to 410 of SEQ ID NO: 29, e.g., from 1 to 6, from 6 to 12, from 12 to 18, from 18 to 24, from 24 to 50, from 50 to 100, from 100 to 150, or from 150 to 200 nucleotides of a binding site within or encompassing nucleotides 261 to 410 of SEQ ID NO: 29, e.g., at least one, at least two, at least three, at least four, at least five, at least six, at least seven, at least eight, at least nine, at least 10, at least 12, at least 15, at least 20, at least 25, at least 30, at least 40, at least 50, at least 60, at least 70, at least 80, at least 90, at least 100, at least 120, at least 150, or at least 200 nucleotides of a binding site within or encompassing nucleotides 261 to 410 of SEQ ID NO: 29). For example, in particular embodiments, the binding site comprises six or more of nucleotides 261 to 410 of SEQ ID NO: 29. In some embodiments, the binding domain comprises six or more consecutive nucleic acid residues that are complementary to (e.g., antisense to) the six or more nucleotides of the binding site. In some embodiments, the binding domain comprises a set of consecutive nucleic acid residues that are complementary to a corresponding set of complementary nucleotides of an ABCA4 binding site having one or more of nucleotides 261 to 410 of SEQ ID NO: 29, wherein the set of consecutive nucleic acid residues of the binding domain is from 6 to 500 residues in length (e.g., from 8 to 400, from 12 to 300, from 16 to 200, from 24 to 280, or from 50 to 150 residues in length, e.g., from 100 to 200, from 6 to 10, from 10 to 20, from 20 to 30, from 30 to 40, from 40 to 50, from 50 to 80, from 80 to 100, from 100 to 120, from 120 to 150, from 150 to 200, or from 200 to 300 residues in length, e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, or more residues in length).

In some embodiments, the binding domain is configured to bind ABCA4 intron 23 at a binding site comprising any one or more of nucleotides 801 to 950 of SEQ ID NO: 29 (e.g., from 1 to 200, from 6 to 150, from 12 to 100, or from 20 to 80 nucleotides of a binding site within or encompassing nucleotides 801 to 950 of SEQ ID NO: 29, e.g., from 1 to 6, from 6 to 12, from 12 to 18, from 18 to 24, from 24 to 50, from 50 to 100, from 100 to 150, or from 150 to 200 nucleotides of a binding site within or encompassing nucleotides 801 to 950 of SEQ ID NO: 29, e.g., at least one, at least two, at least three, at least four, at least five, at least six, at least seven, at least eight, at least nine, at least 10, at least 12, at least 15, at least 20, at least 25, at least 30, at least 40, at least 50, at least 60, at least 70, at least 80, at least 90, at least 100, at least 120, at least 150, or at least 200 nucleotides of a binding site within or encompassing nucleotides 801 to 950 of SEQ ID NO: 29). For example, in particular embodiments, the binding site comprises six or more of nucleotides 801 to 950 of SEQ ID NO: 29. In some embodiments, the binding domain comprises six or more consecutive nucleic acid residues that are complementary to (e.g., antisense to) the six or more nucleotides of the binding site. In some embodiments, the binding domain comprises a set of consecutive nucleic acid residues that are complementary to a corresponding set of complementary nucleotides of an ABCA4 binding site having one or more of nucleotides 801 to 950 of SEQ ID NO: 29, wherein the set of consecutive nucleic acid residues of the binding domain is from 6 to 500 residues in length (e.g., from 8 to 400, from 12 to 300, from 16 to 200, from 24 to 280, or from 50 to 150 residues in length, e.g., from 100 to 200, from 6 to 10, from 10 to 20, from 20 to 30, from 30 to 40, from 40 to 50, from 50 to 80, from 80 to 100, from 100 to 120, from 120 to 150, from 150 to 200, or from 200 to 300 residues in length, e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, or more residues in length).

In some embodiments, the binding domain is configured to bind ABCA4 intron 23 at a binding site comprising any one or more of nucleotides 841 to 990 of SEQ ID NO: 29 (e.g., from 1 to 200, from 6 to 150, from 12 to 100, or from 20 to 80 nucleotides of a binding site within or encompassing nucleotides 841 to 990 of SEQ ID NO: 29, e.g., from 1 to 6, from 6 to 12, from 12 to 18, from 18 to 24, from 24 to 50, from 50 to 100, from 100 to 150, or from 150 to 200 nucleotides of a binding site within or encompassing nucleotides 841 to 990 of SEQ ID NO: 29, e.g., at least one, at least two, at least three, at least four, at least five, at least six, at least seven, at least eight, at least nine, at least 10, at least 12, at least 15, at least 20, at least 25, at least 30, at least 40, at least 50, at least 60, at least 70, at least 80, at least 90, at least 100, at least 120, at least 150, or at least 200 nucleotides of a binding site within or encompassing nucleotides 841 to 990 of SEQ ID NO: 29). For example, in particular embodiments, the binding site comprises six or more of nucleotides 841 to 990 of SEQ ID NO: 29. In some embodiments, the binding domain comprises six or more consecutive nucleic acid residues that are complementary to (e.g., antisense to) the six or more nucleotides of the binding site. In some embodiments, the binding domain comprises a set of consecutive nucleic acid residues that are complementary to a corresponding set of complementary nucleotides of an ABCA4 binding site having one or more of nucleotides 841 to 990 of SEQ ID NO: 29, wherein the set of consecutive nucleic acid residues of the binding domain is from 6 to 500 residues in length (e.g., from 8 to 400, from 12 to 300, from 16 to 200, from 24 to 280, or from 50 to 150 residues in length, e.g., from 100 to 200, from 6 to 10, from 10 to 20, from 20 to 30, from 30 to 40, from 40 to 50, from 50 to 80, from 80 to 100, from 100 to 120, from 120 to 150, from 150 to 200, or from 200 to 300 residues in length, e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, or more residues in length).

In other embodiments, the target ABCA4 intron is intron 24, the mutation is in any one of ABCA4 exons 1-24 or introns 1-24, and the coding domain comprises ABCA4 exons 1-24. In some embodiments, the binding domain is configured to bind intron 24 at a binding site comprising any one or more of nucleotides 600 to 1,250 or nucleotides 1,490 to 2,660 of SEQ ID NO: 30. In other embodiments, the binding site comprises any one or more of nucleotides 1,000 to 1,200 of SEQ ID NO: 30.

In some embodiments, the binding domain binds to the target ABCA4 intron 5′ to the mutation, and wherein the mutation is in any one of ABCA4 exons 23-50 or introns 22-49. For example, in some embodiments, the target ABCA4 intron is intron 23, the mutation is in any one of ABCA4 exons 24-50 or introns 23-49, and the coding domain comprises ABCA4 exons 24-50. In some embodiments, the binding domain is configured to bind intron 23 at a binding site comprising any one or more of nucleotides 80 to 1,081 of SEQ ID NO: 29. In some embodiments, the binding site comprises any one or more of nucleotides 230 to 1,081 of SEQ ID NO: 29, e.g., any one or more of nucleotides 250 to 400 of SEQ ID NO: 29 or any one or more of nucleotides 690 to 850 of SEQ ID NO: 29.

In some embodiments, the target ABCA4 intron is intron 24, the mutation is in any one of ABCA4 exons 25-50 or introns 24-49, and the coding domain comprises ABCA4 exons 25-50. In some embodiments, the binding domain is configured to bind intron 24 at a binding site comprising any one or more of nucleotides 1 to 250, nucleotides 300 to 2,100, or nucleotides 2,200 to 2,692 of SEQ ID NO: 30. In some embodiments, the binding site comprises any one or more of nucleotides 360 to 610 of SEQ ID NO: 30. In other embodiments, the binding site comprises any one or more of nucleotides 750 to 1,110 of SEQ ID NO: 30.

In another aspect, the invention features a nucleic acid trans-splicing molecule comprising, operatively linked in a 5′-to-3′ direction: (a) a binding domain configured to bind ABCA4 intron 22 at a binding site comprising any one or more of nucleotides 60 to 570, 600 to 800, or 900 to 1,350 of SEQ ID NO: 28; (b) a splicing domain configured to mediate trans-splicing; and (c) a coding domain comprising functional ABCA4 exons 23-50; wherein the nucleic acid trans-splicing molecule is configured to trans-splice the coding domain to endogenous ABCA4 exon 22, thereby replacing endogenous ABCA4 exons 23-50 with the functional ABCA4 exons 23-50. In some embodiments, the binding site comprises any one or more of nucleotides 70-250 of SEQ ID NO: 28.

In yet another aspect, the invention features a nucleic acid trans-splicing molecule comprising, operatively linked in a 3′-to-5′ direction: (a) a binding domain configured to bind ABCA4 intron 22 at a binding site comprising any one or more of nucleotides 1 to 510 or 880 to 1,350 of SEQ ID NO: 28; (b) a splicing domain configured to mediate trans-splicing; and (c) a coding domain comprising functional ABCA4 exons 1-22; wherein the nucleic acid trans-splicing molecule is configured to trans-splice the coding domain to endogenous ABCA4 exon 23, thereby replacing endogenous ABCA4 exons 1-22 with the functional ABCA4 exons 1-22.

In some embodiments, the binding domain is configured to bind ABCA4 intron 22 at a binding site comprising any one or more of nucleotides 1041 to 1190 of SEQ ID NO: 28 (e.g., from 1 to 200, from 6 to 150, from 12 to 100, or from 20 to 80 nucleotides of a binding site within or encompassing nucleotides 1041 to 1190 of SEQ ID NO: 28, e.g., from 1 to 6, from 6 to 12, from 12 to 18, from 18 to 24, from 24 to 50, from 50 to 100, from 100 to 150, or from 150 to 200 nucleotides of a binding site within or encompassing nucleotides 1041 to 1190 of SEQ ID NO: 28, e.g., at least one, at least two, at least three, at least four, at least five, at least six, at least seven, at least eight, at least nine, at least 10, at least 12, at least 15, at least 20, at least 25, at least 30, at least 40, at least 50, at least 60, at least 70, at least 80, at least 90, at least 100, at least 120, at least 150, or at least 200 nucleotides of a binding site within or encompassing nucleotides 1041 to 1190 of SEQ ID NO: 28). In particular embodiments, the binding site comprises six or more of nucleotides 1041 to 1190 of SEQ ID NO: 28. In some embodiments, the binding domain comprises six or more consecutive nucleic acid residues that are complementary to (e.g., antisense to) the six or more nucleotides of the binding site. In some embodiments, the binding domain comprises a set of consecutive nucleic acid residues that are complementary to a corresponding set of complementary nucleotides of an ABCA4 binding site having one or more of nucleotides 1041 to 1190 of SEQ ID NO: 28, wherein the set of consecutive nucleic acid residues of the binding domain is from 6 to 500 residues in length (e.g., from 8 to 400, from 12 to 300, from 16 to 200, from 24 to 280, or from 50 to 150 residues in length, e.g., from 100 to 200, from 6 to 10, from 10 to 20, from 20 to 30, from 30 to 40, from 40 to 50, from 50 to 80, from 80 to 100, from 100 to 120, from 120 to 150, from 150 to 200, or from 200 to 300 residues in length, e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, or more residues in length).

In some embodiments, the binding domain is configured to bind any one or more of nucleotides 1171 to 1320 of SEQ ID NO: 28 (e.g., from 1 to 200, from 6 to 150, from 12 to 100, or from 20 to 80 nucleotides of a binding site within or encompassing nucleotides 1171 to 1320 of SEQ ID NO: 28, e.g., from 1 to 6, from 6 to 12, from 12 to 18, from 18 to 24, from 24 to 50, from 50 to 100, from 100 to 150, or from 150 to 200 nucleotides of a binding site within or encompassing nucleotides 1171 to 1320 of SEQ ID NO: 28, e.g., at least one, at least two, at least three, at least four, at least five, at least six, at least seven, at least eight, at least nine, at least 10, at least 12, at least 15, at least 20, at least 25, at least 30, at least 40, at least 50, at least 60, at least 70, at least 80, at least 90, at least 100, at least 120, at least 150, or at least 200 nucleotides of a binding site within or encompassing nucleotides 1171 to 1320 of SEQ ID NO: 28). In particular embodiments, the binding site comprises six or more of nucleotides 1171 to 1320 of SEQ ID NO: 28. In some embodiments, the binding domain comprises six or more consecutive nucleic acid residues that are complementary to (e.g., antisense to) the six or more nucleotides of the binding site. In some embodiments, the binding domain comprises a set of consecutive nucleic acid residues that are complementary to a corresponding set of complementary nucleotides of an ABCA4 binding site having one or more of nucleotides 1171 to 1320 of SEQ ID NO: 28, wherein the set of consecutive nucleic acid residues of the binding domain is from 6 to 500 residues in length (e.g., from 8 to 400, from 12 to 300, from 16 to 200, from 24 to 280, or from 50 to 150 residues in length, e.g., from 100 to 200, from 6 to 10, from 10 to 20, from 20 to 30, from 30 to 40, from 40 to 50, from 50 to 80, from 80 to 100, from 100 to 120, from 120 to 150, from 150 to 200, or from 200 to 300 residues in length, e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, or more residues in length).

In some embodiments, the binding domain is configured to bind any one or more of nucleotides 1201 to 1350 of SEQ ID NO: 28 (e.g., from 1 to 200, from 6 to 150, from 12 to 100, or from 20 to 80 nucleotides of a binding site within or encompassing nucleotides 1201 to 1350 of SEQ ID NO: 28, e.g., from 1 to 6, from 6 to 12, from 12 to 18, from 18 to 24, from 24 to 50, from 50 to 100, from 100 to 150, or from 150 to 200 nucleotides of a binding site within or encompassing nucleotides 1201 to 1350 of SEQ ID NO: 28, e.g., at least one, at least two, at least three, at least four, at least five, at least six, at least seven, at least eight, at least nine, at least 10, at least 12, at least 15, at least 20, at least 25, at least 30, at least 40, at least 50, at least 60, at least 70, at least 80, at least 90, at least 100, at least 120, at least 150, or at least 200 nucleotides of a binding site within or encompassing nucleotides 1201 to 1350 of SEQ ID NO: 28). In particular embodiments, the binding site comprises six or more of nucleotides 1201 to 1350 of SEQ ID NO: 28. In some embodiments, the binding domain comprises six or more consecutive nucleic acid residues that are complementary to (e.g., antisense to) the six or more nucleotides of the binding site. In some embodiments, the binding domain comprises a set of consecutive nucleic acid residues that are complementary to a corresponding set of complementary nucleotides of an ABCA4 binding site having one or more of nucleotides 1201 to 1350 of SEQ ID NO: 28, wherein the set of consecutive nucleic acid residues of the binding domain is from 6 to 500 residues in length (e.g., from 8 to 400, from 12 to 300, from 16 to 200, from 24 to 280, or from 50 to 150 residues in length, e.g., from 100 to 200, from 6 to 10, from 10 to 20, from 20 to 30, from 30 to 40, from 40 to 50, from 50 to 80, from 80 to 100, from 100 to 120, from 120 to 150, from 150 to 200, or from 200 to 300 residues in length, e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, or more residues in length).

In any of the preceding embodiments, the binding domain can be 20-1,000 nucleotides in length (e.g., 25-900 nucleotides in length, 30-800 nucleotides in length, 40-700 nucleotides in length, 50-600 nucleotides in length, 75-500 nucleotides in length, 100-400 nucleotides in length, 125-200 nucleotides in length, or about 150 nucleotides in length, e.g., 20-30 nucleotides in length, 30-40 nucleotides in length, 40-50 nucleotides in length, 50-75 nucleotides in length, 75-100 nucleotides in length, 125-150 nucleotides in length, 150-175 nucleotides in length, 175-200 nucleotides in length, 200-250 nucleotides in length, 250-500 nucleotides in length, 500-750 nucleotides in length, or 750-1,000 nucleotides in length).

In some embodiments, the coding domain is a cDNA sequence. In some embodiments, the coding domain comprises a naturally-occurring sequence. In other embodiments, the coding domain includes a codon-optimized sequence. In some embodiments, the trans-splicing molecule includes an artificial intron that comprises a spacer sequence.

In some embodiments of any of the preceding methods, the nucleic acid trans-splicing molecule is from 3,000 to 4,000 nucleotides in length (e.g., 3,100-3,900 nucleotides in length, 3,200-3,800 nucleotides in length, 3,300-3,700 nucleotides in length, 3,400-3,600 nucleotides in length, or about 3,500 nucleotides in length, e.g., 3,000-3,100 nucleotides in length, 3,100-3,200 nucleotides in length, 3,200-3,300 nucleotides in length, 3,300-3,400 nucleotides in length, 3,400-3,500 nucleotides in length, 3,500-3,600 nucleotides in length, 3,600-3,700 nucleotides in length, 3,800-3,900 nucleotides in length, or 3,900-4,000 nucleotides in length).

In some embodiments, the mutation in the ABCA4 gene is associated with Stargardt Disease. In some embodiments, the mutation in the ABCA4 gene associated with Stargardt Disease is expressed in a photoreceptor cell.

In another aspect, provided herein is a nucleic acid trans-splicing molecule comprising, operatively linked in a 3′-to-5′ direction: (a) a binding domain configured to bind ABCA4 intron 23 at a binding site comprising six or more of nucleotides 261 to 410 of SEQ ID NO: 29, wherein the binding domain comprises six or more consecutive nucleic acid residues that are complementary to the six or more nucleotides of the binding site; (b) an artificial intron comprising a splicing domain; and (c) a coding domain comprising functional ABCA4 exons 1-23; wherein the nucleic acid trans-splicing molecule is configured to trans-splice the coding domain to endogenous ABCA4 exon 24, thereby replacing endogenous ABCA4 exons 1-23 with the functional ABCA4 exons 1-23.

In another aspect, the invention provides a nucleic acid trans-splicing molecule comprising, operatively linked in a 3′-to-5′ direction: (a) a binding domain configured to bind ABCA4 intron 23 at a binding site comprising six or more of nucleotides 801 to 950 of SEQ ID NO: 29, wherein the binding domain comprises six or more consecutive nucleic acid residues that are complementary to the six or more nucleotides of the binding site; (b) an artificial intron comprising a splicing domain; and (c) a coding domain comprising functional ABCA4 exons 1-23; wherein the nucleic acid trans-splicing molecule is configured to trans-splice the coding domain to endogenous ABCA4 exon 24, thereby replacing endogenous ABCA4 exons 1-23 with the functional ABCA4 exons 1-23.

In another aspect, provided herein is a nucleic acid trans-splicing molecule comprising, operatively linked in a 3′-to-5′ direction: (a) a binding domain configured to bind ABCA4 intron 23 at a binding site comprising six or more of nucleotides 841 to 990 of SEQ ID NO: 29, wherein the binding domain comprises six or more consecutive nucleic acid residues that are complementary to the six or more nucleotides of the binding site; (b) an artificial intron comprising a splicing domain; and (c) a coding domain comprising functional ABCA4 exons 1-23; wherein the nucleic acid trans-splicing molecule is configured to trans-splice the coding domain to endogenous ABCA4 exon 24, thereby replacing endogenous ABCA4 exons 1-23 with the functional ABCA4 exons 1-23.

In another aspect, the invention provides a nucleic acid trans-splicing molecule comprising, operatively linked in a 3′-to-5′ direction: (a) a binding domain configured to bind ABCA4 intron 22 at a binding site comprising six or more of nucleotides 1041 to 1190 of SEQ ID NO: 28, wherein the binding domain comprises six or more consecutive nucleic acid residues that are complementary to the six or more nucleotides of the binding site; (b) an artificial intron comprising a splicing domain; and (c) a coding domain comprising functional ABCA4 exons 1-22; wherein the nucleic acid trans-splicing molecule is configured to trans-splice the coding domain to endogenous ABCA4 exon 23, thereby replacing endogenous ABCA4 exons 1-22 with the functional ABCA4 exons 1-22.

In another aspect, the invention features a nucleic acid trans-splicing molecule comprising, operatively linked in a 3′-to-5′ direction: (a) a binding domain configured to bind ABCA4 intron 22 at a binding site comprising six or more of nucleotides 1171 to 1320 of SEQ ID NO: 28, wherein the binding domain comprises six or more consecutive nucleic acid residues that are complementary to the six or more nucleotides of the binding site; (b) an artificial intron comprising a splicing domain; and (c) a coding domain comprising functional ABCA4 exons 1-22; wherein the nucleic acid trans-splicing molecule is configured to trans-splice the coding domain to endogenous ABCA4 exon 23, thereby replacing endogenous ABCA4 exons 1-22 with the functional ABCA4 exons 1-22.

In yet another aspect, provided herein is a nucleic acid trans-splicing molecule comprising, operatively linked in a 3′-to-5′ direction: (a) a binding domain configured to bind ABCA4 intron 22 at a binding site comprising six or more of nucleotides 1201 to 1350 of SEQ ID NO: 28, wherein the binding domain comprises six or more consecutive nucleic acid residues that are complementary to the six or more nucleotides of the binding site; (b) an artificial intron comprising a splicing domain; and (c) a coding domain comprising functional ABCA4 exons 1-22; wherein the nucleic acid trans-splicing molecule is configured to trans-splice the coding domain to endogenous ABCA4 exon 23, thereby replacing endogenous ABCA4 exons 1-22 with the functional ABCA4 exons 1-22.

In another aspect, the invention features a proviral plasmid including the nucleic acid trans-splicing molecule of any of the preceding embodiments.

In yet another aspect, the invention features an adeno-associated virus (AAV) comprising the nucleic acid molecule of any of the preceding embodiments. In some embodiments, the AAV preferentially targets a photoreceptor cell. In some embodiments, the AAV comprises an AAV5 capsid protein, an AAV8 capsid protein, an AAV8(b) capsid protein, or an AAV9 capsid protein.

In another aspect, the invention features a pharmaceutical composition comprising the nucleic acid trans-splicing molecule, the proviral plasmid, or the AAV of any of the preceding aspects.

In another aspect, provided herein is a pharmaceutical composition having any of the 5′ nucleic acid trans-splicing molecules of any the preceding embodiments and a 3′ nucleic acid trans-splicing molecule of any of the preceding embodiments.

In yet another aspect, the invention features a method of correcting a mutation in an ABCA4 gene in a target cell of a subject by administering to the subject the pharmaceutical composition of any of the preceding aspects.

In another aspect, provided herein is a method of correcting a mutation in any one or more of ABCA4 exons 1-24 in a subject in need thereof by administering to the subject a pharmaceutical composition having the nucleic acid trans-splicing molecule of any of the preceding embodiments. In particular embodiments, the mutated ABCA4 exon to be corrected by an ABCA4 trans-splicing molecule of the invention is exon 2. Additionally or alternatively, the mutated ABCA4 exon to be corrected by an ABCA4 trans-splicing molecule of the invention is exon 3. Additionally or alternatively, the mutated ABCA4 exon to be corrected by an ABCA4 trans-splicing molecule of the invention is exon 4.

In another aspect, the invention includes a method of correcting a mutation in any one or more of ABCA4 exons 23-50 in a subject in need thereof by administering to the subject a pharmaceutical composition comprising the nucleic acid trans-splicing molecule of any of the preceding embodiments.

In another aspect, the invention features a method of correcting a mutation in any one of ABCA4 exons 1-24 and a second mutation in any one of exons 23-50 in a subject in need thereof, the method comprising administering to the subject the pharmaceutical composition having a 5′ nucleic acid trans-splicing molecules of any the preceding embodiments and a 3′ nucleic acid trans-splicing molecule of any of the preceding embodiments.

In yet another embodiment, the invention features a method of treating a subject having a disorder associated with a mutation in ABCA4, the method comprising administering to the subject the any of the preceding pharmaceutical compositions. In some embodiments, a subject having a disorder associated with a mutation in any one or more of ABCA4 exons 1-24 or introns 1-24 is treated by administering a pharmaceutical composition comprising the nucleic acid trans-splicing molecule of any of the preceding embodiments. In some embodiments, a subject having a disorder associated with a mutation in any one or more of ABCA4 exons 23-50 or introns 22-49 is treated by administering a pharmaceutical composition comprising the nucleic acid trans-splicing molecule of any of the preceding embodiments.

In another aspect, the invention features a method of treating a subject having a disorder associated with a first mutation in any one of ABCA4 exons 1-24 and a second mutation in any one of exons 23-50 by administering to the subject the pharmaceutical composition having a 5′ nucleic acid trans-splicing molecules of any the preceding embodiments and a 3′ nucleic acid trans-splicing molecule of any of the preceding embodiments.

In any of the preceding methods, the subject may have Stargardt Disease. In some embodiments, the composition is administered by subretinal injection, intravitreal injection, or intravenous injection.

In some embodiments of any of the preceding methods, the subject exhibits at least 1% increase in ABCA4 protein expression after administration (e.g., a 1-5% increase, a 5-10%, a 10-15% increase, a 15-20% increase, a 20-25% increase, a 25-50% increase, or a 50-100% increase in ABCA4 protein expression after administration, e.g., relative to an ABCA4 protein expression in the same subject prior to administration, or relative to a reference sample, reference subject, or a reference group of subjects).

### CEP290

In another aspect, the invention features CEP290 trans-splicing molecules. For example, the invention provides a nucleic acid trans-splicing molecule comprising, operatively linked in a 3′-to-5′ direction: (a) a binding domain configured to bind CEP290 intron 26 at a binding site comprising any one or more of nucleotides 4,800 to 5,838 of SEQ ID NO: 85; (b) a splicing domain configured to mediate trans-splicing; and (c) a coding domain comprising functional CEP290 exons 2-26; wherein the nucleic acid trans-splicing molecule is configured to trans-splice the coding domain to endogenous CEP290 exon 27, thereby replacing endogenous CEP290 exons 2-26 with the functional CEP290 exons 2-26 and correcting the pathogenic point mutation. In some embodiments, the pathogenic point mutation is an A-to-G mutation at nucleotide 1,655 of SEQ ID NO: 85.

In some embodiments, the binding site comprises any one or more of nucleotides 4,980 to 5,838 of SEQ ID NO: 85. In some embodiments, the binding site comprises any one or more of nucleotides 5,348 to 5,838 of SEQ ID NO: 85. In some embodiments, the binding site comprises any one or more of nucleotides 5,348 to 5,700 of SEQ ID NO: 85. In some embodiments, the binding site comprises any one or more of nucleotides 5,400 to 5,600 of SEQ ID NO: 85. In some embodiments, the binding site comprises any one or more of nucleotides 5,460 to 5,560 of SEQ ID NO: 85. In some embodiments, the binding site comprises nucleotide 5,500 of SEQ ID NO: 85.

In another aspect, the invention features a nucleic acid trans-splicing molecule comprising, operatively linked in a 3′-to-5′ direction: (a) a binding domain configured to bind CEP290 at any one of target introns 27, 28, 29, or 30; (b) a splicing domain configured to mediate trans-splicing; and (c) a coding domain comprising functional CEP290 exons 5′ to the target intron; wherein the nucleic acid trans-splicing molecule is configured to trans-splice the coding domain to endogenous CEP290, thereby replacing endogenous CEP290 exons 5′ to the target intron with the functional CEP290 exons and correcting the pathogenic point mutation. In some embodiments, the pathogenic point mutation is an A-to-G mutation at nucleotide 1,655 of SEQ ID NO: 85.

In some embodiments, the target intron is intron 27, the coding domain comprising functional CEP290 exons 2-27, and the nucleic acid trans-splicing molecule is configured to replace endogenous CEP290 exons 2-27 with the functional CEP290 exons 2-27. In some embodiments, the binding domain is configured to bind intron 27 at a binding site comprising any one or more of nucleotides 120 to 680, nucleotides 710 to 2,200, or nucleotides 2,670 to 2,910 of SEQ ID NO: 86. In some embodiments, the binding site comprises any one or more of nucleotides 790 to 2,100 of SEQ ID NO: 86, e.g., any one or more of nucleotides 1,020 to 1,630 of SEQ ID NO: 86. In other embodiments, the binding site comprises any one or more of nucleotides 1,670 to 2,000 of SEQ ID NO: 86.

In some embodiments, the target intron is intron 28, the coding domain comprising functional CEP290 exons 2-28, and the nucleic acid trans-splicing molecule is configured to replace endogenous CEP290 exons 2-28 with the functional CEP290 exons 2-28. In some embodiments, the binding domain is configured to bind intron 28 at a binding site comprising any one or more of nucleotides 1 to 390, nucleotides 410 to 560, or nucleotides 730 to 937 of SEQ ID NO: 87. In some embodiments, the binding site comprises any one or more of nucleotides 1 to 200 of SEQ ID NO: 87. In other embodiments, the binding site comprises any one or more of nucleotides 720 to 900 of SEQ ID NO: 87.

In some embodiments, the target intron is intron 29, the coding domain comprising functional CEP290 exons 2-29, and the nucleic acid trans-splicing molecule is configured to replace endogenous CEP290 exons 2-29 with the functional CEP290 exons 2-29. In some embodiments, the binding domain is configured to bind intron 29 at a binding site comprising any one or more of nucleotides 1 to 600, nucleotides 720 to 940, or nucleotides 1,370 to 1,790 of SEQ ID NO: 88.

In some embodiments, the target intron is intron 30, the coding domain comprising functional CEP290 exons 2-30, and the nucleic acid trans-splicing molecule is configured to replace endogenous CEP290 exons 2-30 with the functional CEP290 exons 2-30. In some embodiments, the binding domain is configured to bind intron 29 at a binding site comprising any one or more of nucleotides 880 to 1,240 of SEQ ID NO: 89, e.g., any one or more of nucleotides 950 to 1,240 of SEQ ID NO: 89, e.g., any one or more of nucleotides 1,060 to 1,240 of SEQ ID NO: 89.

In any of the preceding embodiments, the binding domain is 20-1,000 nucleotides in length (e.g., 25-900 nucleotides in length, 30-800 nucleotides in length, 40-700 nucleotides in length, 50-600 nucleotides in length, 75-500 nucleotides in length, 100-400 nucleotides in length, 125-200 nucleotides in length, or about 150 nucleotides in length, e.g., 20-30 nucleotides in length, 30-40 nucleotides in length, 40-50 nucleotides in length, 50-75 nucleotides in length, 75-100 nucleotides in length, 125-150 nucleotides in length, 150-175 nucleotides in length, 175-200 nucleotides in length, 200-250 nucleotides in length, 250-500 nucleotides in length, 500-750 nucleotides in length, or 750-1,000 nucleotides in length).

In some embodiments, the coding domain is a cDNA sequence. In some embodiments, the coding domain is a naturally-occurring sequence. In other embodiments, the coding domain is a codon-optimized sequence.

In some embodiments, an artificial intron comprises an artificial intron and a spacer sequence.

In any of the preceding embodiments, the nucleic acid trans-splicing molecule may be 3,000 to 4,000 nucleotides in length.

In any of the preceding embodiments, the mutated CEP290 exon may be associated with LCA 10. In some embodiments, the mutated CEP290 exon associated with LCA 10 is expressed in a photoreceptor cell.

In another aspect of the invention, provided herein is a proviral plasmid comprising the nucleic acid trans-splicing molecule of any of the preceding aspects.

In yet another aspect, the invention provides an adeno-associated virus (AAV) comprising the nucleic acid molecule of any of the preceding aspects. In some embodiments, the AAV preferentially targets a photoreceptor cell. In some embodiments, the AAV comprises an AAV5 capsid protein, an AAV8 capsid protein, an AAV8(b) capsid protein, or an AAV9 capsid protein.

In another aspect, the invention features a pharmaceutical composition comprising the nucleic acid trans-splicing molecule, the proviral plasmid, or the AAV of any of the preceding aspects.

In another aspect, featured herein are methods of correcting a pathogenic point mutation in CEP290 intron 26 in a target cell of a subject, the methods comprising administering to the subject the nucleic acid trans-splicing molecule, the proviral plasmid, the AAV, or the pharmaceutical composition of any of the preceding aspects. In some embodiments, the subject has LCA 10.

In yet another aspect, the invention provides a method of treating a subject having LCA 10 caused by a pathogenic point mutation in CEP290 intron 26, the method comprising administering to the subject the nucleic acid trans-splicing molecule, the proviral plasmid, the AAV, or the pharmaceutical composition of any of the preceding aspects.

In any of the preceding methods, the pathogenic point mutation may be an A-to-G mutation at nucleotide 1,655 of CEP290 intron 26 (SEQ ID NO: 85). In some embodiments, the nucleic acid trans-splicing molecule, the proviral plasmid, the AAV, or the pharmaceutical composition is administered by subretinal injection, intravitreal injection, or intravenous injection.

In another aspect, the invention provides a kit comprising any one or more of the aforementioned nucleic acid trans-splicing molecules, proviral plasmids, AAVs, or pharmaceutical compositions, wherein the kit further includes instructions for using the one or more nucleic acid trans-splicing molecules, proviral plasmids, AAVs, or pharmaceutical compositions for correcting a mutation in a CEP290 gene of a subject (e.g., a mutation associated with a disorder, such as LCA 10).

### BRIEF DESCRIPTION OF THE DRAWINGS

FIG. 1 is a schematic drawing of several exemplary nucleic acid trans-splicing molecules for correcting a mutated ABCA4 exon with a functional ABCA4 exon. Dark shaded boxes represent native ABCA4 exons. Dashed lines joining the dark shaded boxes represent native introns. Light shaded boxes with dark borders represent functional ABCA4 exons within a nucleic acid trans-splicing molecule. A splicing domain, represented by a curved line, is attached to one end of each of the functional ABCA4 exons and leads to an intron of the ABCA4 pre-mRNA.

FIG. 2 is a graph showing trans-splicing efficiency (relative fold change) conferred by 150-mer binding domains across ABCA4 intron 19 (SEQ ID NO: 25) in ten-nucleotide intervals using 5′ trans-splicing molecules. X axis labels indicate the number of each binding site starting from the 5′ end of the intron (i.e., the first nucleotide of the intron sequence).

FIG. 3 is a graph showing trans-splicing efficiency (relative fold change) conferred by 150-mer binding domains across ABCA4 intron 22 (SEQ ID NO: 28) in ton-nucleotide intervals using 5′ trans-splicing molecules. X axis labels indicate the number of each binding site starting from the 5′ end of the intron (i.e., the first nucleotide of the intron sequence).

FIG. 4 is a graph showing trans-splicing efficiency (relative fold change) conferred by 150-mer binding domains across ABCA4 intron 22 (SEQ ID NO: 28) in ten-nucleotide intervals using 3′ trans-splicing molecules. X axis labels indicate the number of each binding site starting from the 5′ end of the intron (i.e., the first nucleotide of the intron sequence).

FIG. 5 is a graph showing trans-splicing efficiency (relative fold change) conferred by 150-mer binding domains across ABCA4 intron 23 (SEQ ID NO: 29) in ten-nucleotide intervals using 5′ trans-splicing molecules. X axis labels indicate the number of each binding site starting from the 5′ end of the intron (i.e., the first nucleotide of the intron sequence).

FIG. 6 is a graph showing trans-splicing efficiency (relative fold change) conferred by 150-mer binding domains across ABCA4 intron 23 (SEQ ID NO: 29) in ten-nucleotide intervals using 3′ trans-splicing molecules. X axis labels indicate the number of each binding site starting from the 5′ end of the intron (i.e., the first nucleotide of the intron sequence).

FIG. 7 is a graph showing trans-splicing efficiency (relative fold change) conferred by 150-mer binding domains across ABCA4 intron 24 (SEQ ID NO: 30) in ten-nucleotide intervals using 5′ trans-splicing molecules. X axis labels indicate the number of each binding site starting from the 5′ end of the intron (i.e., the first nucleotide of the intron sequence).

FIG. 8 is a graph showing trans-splicing efficiency (relative fold change) conferred by 150-mer binding domains across ABCA4 intron 24 (SEQ ID NO: 30) in ten-nucleotide intervals using 3′ trans-splicing molecules. X axis labels indicate the number of each binding site starting from the 5′ end of the intron (i.e., the first nucleotide of the intron sequence).

FIG. 9 is a schematic drawing showing a TALEN protein consisting of a DNA binding domain linked to a transcription activation domain. A VP64 transcription activation domain is shown. The right panel shows a portion of the 5′ untranslated region (5′-UTR) of ABCA4. The TATA box and the putative transcription start site are shown. The sequences targeted the by the three different DNA binding domains of TALENs are also shown. TALEN 1 binds to the first underlined sequence, TALEN 2 binds to the second underlined sequence, and TALEN 3 binds to the third underlined sequence, as indicated.

FIG. 10 is a gel showing 293T cells were transfected with TALEN constructs designed to induce endogenous ABCA4 expression. All three TALENS from FIG. 9 were stably introduced into 293 cells and single cell clones were picked and analyzed by western blot. The positive control (+) indicates cells transfected with a plasmid expressing an ABCA4 cDNA. Cell lysates were made 48 hours after transfection and the membrane fractions were examined for ABCA4 expression using antibody ab72955 (Abcam). Clones ZT-22 and ZT-48 showed ABCA4 protein expression.

FIG. 11 is a schematic drawing showing a CAG promoter cell line.

FIGS. 12A and 12B show site-specific guides (FIG. 12A) that were designed to insert the CAG promotor and a puromycin selectable marker using homology arms (FIG. 12B).

FIG. 13 is a schematic drawing showing a CAG promoter cell line.

FIGS. 14A and 14B are a graph and a gel, respectively, showing expression results from several clonal lines that were selected for further analyses. FIG. 14A shows RNA expression and FIG. 14B shows protein expression of the cell lines. Membrane preparations of the indicated cells lines were probed for ABCA4 protein using a rabbit polyclonal antibody to ABCA4 (Abcam, ab72955). Exposure time is 23 seconds. 293 cells are the parental cell that does not express ABCA4. The top band is non-specific background present in all cells.

FIG. 15 is a schematic drawing showing CRISPR guide RNA for targeting exons 3 and 4.

FIG. 16 is a graph showing RNA expression and a gel showing protein profiles of single cell clones derived after treatment with CRISPR/Cas9, as depicted in FIG. 15.

FIGS. 17A and 17B are schematic drawings showing PCR for mutation analyses on cDNA (FIG. 17A) and PCR for genotyping on cDNA (FIG. 17B), confirming that exons 3 and 4 were targeted and interrupted.

FIG. 18 is a set of tables showing that the mutation analyses from FIGS. 17A and 17B confirmed that exons 3 and 4 were targeted and interrupted in alleles in the 17+06 and 17+21 cell lines.

FIGS. 19A and 19B are schematic drawings of trans-splicing molecules targeting ABCA4 pre-mRNA. FIG. 19A shows a generic trans-splicing molecule including a codon optimized exon (or set of exons), a binding domain that hybridizes to a target RNA, and an artificial intron linker. FIG. 19B shows various trans-splicing molecules that target particular regions within introns 22 and 23 of ABCA4.

FIGS. 20A-20D are gels (FIGS. 20A and 20C) and graphs (FIGS. 20B and 20D) showing results from trans-splicing reactions. FIGS. 20A and 20B show protein and RNA levels, respectively, of intron 22 trans-splicing reactions, and FIGS. 20C and 20D show protein and RNA levels, respectively, of intron 23 trans-splicing reactions.

FIG. 21 is a schematic drawing of several exemplary nucleic acid trans-splicing molecules for correcting a mutation in CEP290 intron 26 with a functional 5′ portion of the CEP290 gene. Dark shaded boxes represent native CEP290 exons. Dashed lines joining the dark shaded boxes represent native introns. Light shaded boxes with dark borders represent functional CEP290 exons within a nucleic acid trans-splicing molecule. A splicing domain, represented by a curved line, is attached to one end of each of the functional CEP290 exon sequences and leads to an intron of the CEP290 pre-mRNA.

FIG. 22 is a graph showing trans-splicing efficiency (relative fold change) conferred by 150-mer binding domains across CEP290 intron 26 (SEQ ID NO: 85) in ten-nucleotide intervals. X axis labels indicate “motif number,” of the number of each binding site starting from the 5′ end of the intron (i.e., the first nucleotide of the intron sequence).

FIG. 23 is a graph showing trans-splicing efficiency (relative fold change) conferred by 150-mer binding domains across CEP290 intron 27 (SEQ ID NO: 86) in ten-nucleotide intervals. Each of the three lines represents an independent experiment. X axis labels indicate “motif number,” of the number of each binding site starting from the 5′ end of the intron (i.e., the first nucleotide of the intron sequence).

FIG. 24 is a graph showing trans-splicing efficiency (relative fold change) conferred by 150-mer binding domains across CEP290 intron 28 (SEQ ID NO: 87) in ten-nucleotide intervals. Each of the three lines represents an independent experiment. X axis labels indicate “motif number,” of the number of each binding site starting from the 5′ end of the intron (i.e., the first nucleotide of the intron sequence).

FIG. 25 is a graph showing trans-splicing efficiency (relative fold change) conferred by 150-mer binding domains across CEP290 intron 29 (SEQ ID NO: 88) in ten-nucleotide intervals. Each of the three lines represents an independent experiment. X axis labels indicate “motif number,” of the number of each binding site starting from the 5′ end of the intron (i.e., the first nucleotide of the intron sequence).

FIG. 26 is a graph showing trans-splicing efficiency (relative fold change) conferred by 150-mer binding domains across CEP290 intron 30 (SEQ ID NO: 89) in ten-nucleotide intervals. Each of the three lines represents an independent experiment. X axis labels indicate “motif number,” of the number of each binding site starting from the 5′ end of the intron (i.e., the first nucleotide of the intron sequence).

### DETAILED DESCRIPTION

The compositions and methods described herein involve trans-splicing molecules (e.g., pre-mRNA trans-splicing molecules delivered by adeno-associated virus (AAV)) for treating diseases or disorders caused by a mutation in the ABCA4 gene. The methods and compositions described herein employ pre-mRNA trans-splicing as a gene therapy (e.g., ex vivo and in vivo gene therapy) for the treatment of diseases caused by an ABCA4 mutation, such as Stargardt Disease (e.g., Stargardt Disease 1).

Alternatively, compositions and methods described herein involve trans-splicing molecules (e.g., pre-mRNA trans-splicing molecules delivered by adeno-associated virus (AAV)) for treating diseases or disorders caused by a mutation in the CEP290 gene, such as LCA 10. These methods employ pre-mRNA trans-splicing as a gene therapy (e.g., ex vivo and in vivo gene therapy) for the treatment of diseases caused by a CEP290 mutation, such as LCA 10.

The trans-splicing molecules and methods of use thereof exemplified herein provide several advantages over conventional therapies. First, the use of the trans-splicing molecule delivery by AAV provides efficient and specific delivery of a gene therapy to photoreceptors, while overcoming difficulties associated with the packaging limit of AAV. Second, these compositions and methods permit correction of the genetic defect at the source. Additionally, the compositions and methods provided herein are useful to treat any type of mutation in ABCA4 (or other large cDNAs/transgene cassettes). Correction of the defect in photoreceptors provides secondary rescue to retinal pigment epithelium cells. Further, the present methods and compositions are generally immunologically benign. The use of subretinal delivery and other features renders the effect specific to target cells, such as photoreceptors, so that toxicity due to off-target splicing is reduced. Further, unlike nucleases, trans-splicing does not require genomic alterations. Finally, RNA repair does not require cell division, whereas DNA repair methodologies (such as CRISPR-Cas9 or zinc fingers) have a requirement for the cell to go through mitosis for homology directed repair to occur, which is a disadvantage in post-mitotic tissues like the retina.

### I. Definitions

Unless defined otherwise, technical and scientific terms used herein have the same meaning as commonly understood by one of ordinary skill in the art to which this invention belongs and by reference to published texts, which provide one skilled in the art with a general guide to many of the terms used in the present application. In the event of any conflicting definitions between those set forth herein and those of a referenced publication, the definition provided herein shall control.

A “nucleic acid trans-splicing molecule” or “trans-splicing molecule” has three main elements: (a) a binding domain that confers specificity by tethering the trans-splicing molecule to its target gene (e.g., pre-mRNA); (b) a splicing domain (e.g., a splicing domain having a 3′ or 5′ splice site); and (c) a coding sequence configured to be trans-spliced onto the target gene, which can replace one or more exons in the target gene (e.g., one or more mutated exons). A “pre-mRNA trans-splicing molecule” or “RTM” refers to a nucleic acid trans-splicing molecule that targets pre-mRNA. In some embodiments, a trans-splicing molecule, such as an RTM, can include cDNA, e.g., as part of a functional exon (e.g., a functional ABCA4 or CEP290 exon, e.g., a codon-optimized exon) for replacement or correction of a mutated ABCA4 exon or CEP290).

By “trans-splicing” is meant joining of a nucleic acid molecule containing one or more exons (e.g., exogenous exons, e.g., exons that are part of a coding domain of a trans-splicing molecule) to a first portion of a separate RNA molecule (e.g., a pre-mRNA molecule, e.g., an endogenous pre-mRNA molecule) by replacing a second portion of the RNA molecule through a spliceosome-mediated mechanism.

“Binding” between a binding domain and a target intron, as used herein, refers to hydrogen bonding between the binding domain and the target intron in a degree sufficient to mediate trans-splicing by bringing the trans-splicing molecule into association with the target gene (e.g., pre-mRNA). In some embodiments, the hydrogen bonds between the binding domain and the target intron are between nucleotide bases that are complementary to and in antisense orientation from one another (e.g., hybridized to one another).

As used herein, an “artificial intron” refers to a nucleic acid sequence that links (directly or indirectly) a binding domain to a coding domain. An artificial intron includes a splicing domain and may further include one or more spacer sequences and/or other regulatory elements.

A “splicing domain,” as used herein, refers to a nucleic acid sequence having motifs that are recognized by the spliceosome and mediate trans-splicing. A splicing domain includes a splice site (e.g., a single splice site, i.e., one and only one splice site), which can be a 3′ splice site or a 5′ splice site. A splicing domain may include other regulatory elements. For example, in some embodiments, a splicing domain includes splicing enhancers (e.g., exonic splicing enhancers (ESE) or intronic splicing enhancers (ISE)). In some embodiments, a splicing domain includes a branch point (e.g., a strong conserved branch point) or branch site sequence and/or a polypyrimidine tract (PPT). In some embodiments, a splicing domain of a 5′ trans-splicing molecule does not contain the branch point or PPT, but comprises a 5′ splice acceptor or a 3′ splice donor.

As used herein, a “mutation” refers to any aberrant nucleic acid sequence that causes a defective protein product (e.g., a non-functional protein product, a protein product having reduced function, a protein product having aberrant function, and/or a protein product that is produced in less than normal or greater than normal quantities). Mutations include base pair mutations (e.g., single nucleotide polymorphisms), missense mutations, frameshift mutations, deletions, insertions, and splice mutations. In some embodiments, a mutation refers to a nucleic acid sequence that is different in one or more portions of its sequence than a corresponding wildtype nucleic acid sequence or functional variant thereof. In some embodiments, a mutation refers to a nucleic acid sequence that encodes a protein having an amino acid sequence that is different than a corresponding wildtype protein or functional variant thereof. A “mutated exon” (e.g., a mutated ABCA4 exon) refers to an exon containing a mutation or an exon sequence that reflects a mutation in a different region, such as a cryptic exon resulting from a mutation in an intron.

As used herein, the term “ABCA4” refers to a polynucleotide (e.g., RNA (e.g., pre-mRNA or mRNA) or DNA) that encodes retinal-specific ATP-binding cassette transporter. An exemplary pre-mRNA sequence of a functional human ABCA4 gene is given by SEQ ID NO: 6. An exemplary genomic DNA sequence of a functional (wildtype) human ABCA4 gene is given by NCBI Reference Sequence: NG_009073. The amino acid sequence of an exemplary ABCA4 protein is given by Protein Accession No. P78363.

Exons and introns of ABCA4 are identified herein as set forth in Table 1, below, which can be mapped onto the ABCA4 pre-mRNA molecule of SEQ ID NO: 6. Each exon and intron of ABCA4 are identified herein according to the reference number in the first (left-hand) column. The size of each exon and intron (base pairs; bp) are indicated in the second and third columns. The fourth column indicates the length of a cDNA molecule corresponding to exons 5′ to the corresponding intron number. The fifth column indicates the length of a cDNA molecule corresponding to mRNA 3′ to the corresponding intron number.

TABLE 1
ABCA4 exon and intron summary

| Exon/Intron | | | | |
|---|---|---|---|---|
| Number | Exon Size | Intron Size | 5′ cDNA | 3′ cDNA |
| 1 | 153 | 7,913 | 66 | 6,822 |
| 2 | 94 | 1,393 | 160 | 6,756 |
| 3 | 142 | 2,721 | 302 | 6,662 |
| 4 | 140 | 5,434 | 442 | 6,520 |
| 5 | 128 | 4,023 | 570 | 6,380 |
| 6 | 198 | 15,352 | 768 | 6,252 |
| 7 | 90 | 2,633 | 858 | 6,054 |
| 8 | 241 | 1,016 | 1,099 | 5,964 |
| 9 | 140 | 615 | 1,239 | 5,723 |
| 10 | 117 | 702 | 1,356 | 5,583 |
| 11 | 198 | 14,372 | 1,554 | 5,466 |
| 12 | 206 | 358 | 1,760 | 5,268 |
| 13 | 177 | 1,817 | 1,937 | 5,062 |
| 14 | 223 | 3,714 | 2,160 | 4,885 |
| 15 | 222 | 1,285 | 2,382 | 4,662 |
| 16 | 205 | 3,412 | 2,587 | 4,440 |
| 17 | 66 | 2,675 | 2,653 | 4,235 |
| 18 | 90 | 1,774 | 2,743 | 4,169 |
| 19 | 175 | 2,174 | 2,918 | 4,079 |
| 20 | 132 | 1,137 | 3,050 | 3,904 |
| 21 | 140 | 437 | 3,190 | 3,772 |
| 22 | 138 | 1,358 | 3,328 | 3,632 |
| 23 | 194 | 1,081 | 3,522 | 3,494 |
| 24 | 85 | 2,692 | 3,607 | 3,300 |
| 25 | 206 | 356 | 3,813 | 3,215 |
| 26 | 49 | 4,696 | 3,862 | 3,009 |
| 27 | 266 | 657 | 4,128 | 2,960 |
| 28 | 125 | 469 | 4,253 | 2,694 |
| 29 | 99 | 796 | 4,352 | 2,569 |
| 30 | 187 | 4,396 | 4,539 | 2,470 |
| 31 | 95 | 1,535 | 4,634 | 2,283 |
| 32 | 33 | 1,434 | 4,667 | 2,188 |
| 33 | 106 | 131 | 4,773 | 2,155 |
| 34 | 75 | 230 | 4,848 | 2,049 |
| 35 | 170 | 1,480 | 5,018 | 1,974 |
| 36 | 178 | 3,727 | 5,196 | 1,804 |
| 37 | 116 | 1,048 | 5,312 | 1,626 |
| 38 | 148 | 3,157 | 5,460 | 1,510 |
| 39 | 124 | 332 | 5,584 | 1,362 |
| 40 | 130 | 1,928 | 5,714 | 1,238 |
| 41 | 121 | 453 | 5,835 | 1,108 |
| 42 | 63 | 494 | 5,898 | 987 |
| 43 | 107 | 2,051 | 6,005 | 924 |
| 44 | 142 | 3,448 | 6,147 | 817 |
| 45 | 135 | 752 | 6,282 | 675 |
| 46 | 104 | 73 | 6,386 | 540 |
| 47 | 93 | 2,725 | 6,479 | 436 |
| 48 | 250 | 1,665 | 6,729 | 343 |
| 49 | 87 | 2,866 | 6,816 | 93 |
| 50 | 406 | | 6,822 | 6 |

As used herein, a “target ABCA4 intron” refers to one of the 49 ABCA4 introns identified in Table 1, above. Nucleic acid sequence identifiers for each ABCA4 intron sequence are provided in Table 2, below. It will be understood that the scope of the term “target ABCA4 intron” encompasses variants of ABCA4 introns provided herein, such as intron sequences having 90-100% homology with the sequences provided herein (e.g., 90%, 91%, 92%, 93%, 94%, 95%, 96%, 97%, 98%, 99%, or 100% homology with the sequences provided herein), where the location of the variant intron on the ABCA4 gene corresponds with that provided herein (e.g., in relation to its adjacent exons as set forth in Table 1).

TABLE 2
ABCA4 intron sequences

| Intron Number | Sequence |
|---|---|
| 1 | SEQ ID NO: 7 |
| 2 | SEQ ID NO: 8 |
| 3 | SEQ ID NO: 9 |
| 4 | SEQ ID NO: 10 |
| 5 | SEQ ID NO: 11 |
| 6 | SEQ ID NO: 12 |
| 7 | SEQ ID NO: 13 |
| 8 | SEQ ID NO: 14 |
| 9 | SEQ ID NO: 15 |
| 10 | SEQ ID NO: 16 |
| 11 | SEQ ID NO: 17 |
| 12 | SEQ ID NO: 18 |
| 13 | SEQ ID NO: 19 |
| 14 | SEQ ID NO: 20 |
| 15 | SEQ ID NO: 21 |
| 16 | SEQ ID NO: 22 |
| 17 | SEQ ID NO: 23 |
| 18 | SEQ ID NO: 24 |
| 19 | SEQ ID NO: 25 |
| 20 | SEQ ID NO: 26 |
| 21 | SEQ ID NO: 27 |
| 22 | SEQ ID NO: 28 |
| 23 | SEQ ID NO: 29 |
| 24 | SEQ ID NO: 30 |
| 25 | SEQ ID NO: 31 |
| 26 | SEQ ID NO: 32 |
| 27 | SEQ ID NO: 33 |
| 28 | SEQ ID NO: 34 |
| 29 | SEQ ID NO: 35 |
| 30 | SEQ ID NO: 36 |
| 31 | SEQ ID NO: 37 |
| 32 | SEQ ID NO: 38 |
| 33 | SEQ ID NO: 39 |
| 34 | SEQ ID NO: 40 |
| 35 | SEQ ID NO: 41 |
| 36 | SEQ ID NO: 42 |
| 37 | SEQ ID NO: 43 |
| 38 | SEQ ID NO: 44 |
| 39 | SEQ ID NO: 45 |
| 40 | SEQ ID NO: 46 |
| 41 | SEQ ID NO: 47 |
| 42 | SEQ ID NO: 48 |
| 43 | SEQ ID NO: 49 |
| 44 | SEQ ID NO: 50 |
| 45 | SEQ ID NO: 51 |
| 46 | SEQ ID NO: 52 |
| 47 | SEQ ID NO: 53 |
| 48 | SEQ ID NO: 54 |
| 49 | SEQ ID NO: 55 |

As used herein, the term “CEP290” refers to a polynucleotide (e.g., RNA (e.g., pre-mRNA or mRNA) or DNA) that encodes the centrosomal protein 290. An exemplary pre-mRNA sequence of a functional human CEP290 gene is given by SEQ ID NO: 113. An exemplary genomic DNA sequence of a functional (wildtype) human CEP290 gene is given by NCBI Reference Sequence: NG_008417. The amino acid sequence of an exemplary human centrosomal protein 290 protein is given by Protein Accession No. O15078.

Exons and introns of CEP290 are identified herein as set forth in Table 3, below, which can be mapped onto the CEP290 pre-mRNA molecule of SEQ ID NO: 112. Each exon and intron of CEP290 are identified herein according to the reference number in the first (left-hand) column. The size of each exon and intron (base pairs; bp) are indicated in the second and third columns. The fourth column indicates the length of a cDNA molecule corresponding to exons 5′ to the corresponding intron number. The fifth column indicates the length of a cDNA molecule corresponding to mRNA 3′ to the corresponding intron number.

TABLE 3
CEP290 exon and intron summary

| Exon/Intron | | | | |
|---|---|---|---|---|
| Number | Exon size | Intron size | 5′ cDNA | 3′ cDNA |
| 1 | 317 | 565 | N/A | 7440 |
| 2 | 129 | 172 | 102 | 7338 |
| 3 | 78 | 1391 | 180 | 7260 |
| 4 | 70 | 303 | 250 | 7190 |
| 5 | 47 | 2358 | 297 | 7143 |
| 6 | 144 | 5424 | 441 | 6999 |
| 7 | 54 | 599 | 495 | 6945 |
| 8 | 21 | 124 | 516 | 6924 |
| 9 | 153 | 391 | 669 | 6771 |
| 10 | 183 | 658 | 852 | 6588 |
| 11 | 90 | 2507 | 942 | 6498 |
| 12 | 123 | 946 | 1065 | 6375 |
| 13 | 124 | 4079 | 1189 | 6251 |
| 14 | 170 | 720 | 1359 | 6081 |
| 15 | 163 | 1370 | 1522 | 5918 |
| 16 | 101 | 72 | 1623 | 5817 |
| 17 | 88 | 1337 | 1711 | 5729 |
| 18 | 113 | 1850 | 1824 | 5616 |
| 19 | 85 | 535 | 1909 | 5531 |
| 20 | 143 | 2561 | 2052 | 5388 |
| 21 | 165 | 342 | 2217 | 5223 |
| 22 | 150 | 2020 | 2367 | 5073 |
| 23 | 116 | 1967 | 2483 | 4957 |
| 24 | 103 | 90 | 2586 | 4854 |
| 25 | 231 | 3663 | 2817 | 4623 |
| 26 | 174 | 5838 | 2991 | 4449 |
| 27 | 112 | 2912 | 3103 | 4337 |
| 28 | 206 | 937 | 3309 | 4131 |
| 29 | 152 | 1841 | 3461 | 3979 |
| 30 | 112 | 1240 | 3573 | 3867 |
| 31 | 456 | 1087 | 4029 | 3411 |
| 32 | 165 | 1281 | 4194 | 3246 |
| 33 | 108 | 217 | 4302 | 3138 |
| 34 | 135 | 1186 | 4437 | 3003 |
| 35 | 267 | 631 | 4704 | 2736 |
| 36 | 108 | 616 | 4812 | 2628 |
| 37 | 200 | 2635 | 5012 | 2428 |
| 38 | 214 | 952 | 5226 | 2214 |
| 39 | 138 | 1173 | 5364 | 2076 |
| 40 | 222 | 352 | 5586 | 1854 |
| 41 | 123 | 5295 | 5709 | 1731 |
| 42 | 146 | 331 | 5855 | 1585 |
| 43 | 156 | 2648 | 6011 | 1429 |
| 44 | 124 | 4406 | 6135 | 1305 |
| 45 | 135 | 1202 | 6270 | 1170 |
| 46 | 87 | 1697 | 6357 | 1083 |
| 47 | 165 | 809 | 6522 | 918 |
| 48 | 123 | 877 | 6645 | 795 |
| 49 | 173 | 3130 | 6818 | 622 |
| 50 | 142 | 1162 | 6960 | 480 |
| 51 | 74 | 593 | 7034 | 406 |
| 52 | 95 | 3218 | 7129 | 311 |
| 53 | 80 | 939 | 7209 | 231 |
| 54 | 395 | N/A | 7440 | N/A |

As used herein, a “target CEP290 intron” refers to one of the 53 CEP290 introns identified in Table 3, above. Nucleic acid sequence identifiers for each CEP290 intron sequence are provided in Table 4, below. It will be understood that the scope of the term “target CEP290 intron” encompasses variants of CEP290 introns provided herein, such as intron sequences having 90-100% homology with the sequences provided herein (e.g., 90%, 91%, 92%, 93%, 94%, 95%, 96%, 97%, 98%, 99%, or 100% homology with the sequences provided herein), where the location of the variant intron on the CEP290 gene corresponds with that provided herein (e.g., in relation to its adjacent exons as set forth in Table 3).

TABLE 4
CEP290 intron sequences

| Intron Number | Sequence |
|---|---|
| 1 | SEQ ID NO: 60 |
| 2 | SEQ ID NO: 61 |
| 3 | SEQ ID NO: 62 |
| 4 | SEQ ID NO: 63 |
| 5 | SEQ ID NO: 64 |
| 6 | SEQ ID NO: 65 |
| 7 | SEQ ID NO: 66 |
| 8 | SEQ ID NO: 67 |
| 9 | SEQ ID NO: 68 |
| 10 | SEQ ID NO: 69 |
| 11 | SEQ ID NO: 70 |
| 12 | SEQ ID NO: 71 |
| 13 | SEQ ID NO: 72 |
| 14 | SEQ ID NO: 73 |
| 15 | SEQ ID NO: 74 |
| 16 | SEQ ID NO: 75 |
| 17 | SEQ ID NO: 76 |
| 18 | SEQ ID NO: 77 |
| 19 | SEQ ID NO: 78 |
| 20 | SEQ ID NO: 79 |
| 21 | SEQ ID NO: 80 |
| 22 | SEQ ID NO: 81 |
| 23 | SEQ ID NO: 82 |
| 24 | SEQ ID NO: 83 |
| 25 | SEQ ID NO: 84 |
| 26 | SEQ ID NO: 85 |
| 27 | SEQ ID NO: 86 |
| 28 | SEQ ID NO: 87 |
| 29 | SEQ ID NO: 88 |
| 30 | SEQ ID NO: 89 |
| 31 | SEQ ID NO: 90 |
| 32 | SEQ ID NO: 91 |
| 33 | SEQ ID NO: 92 |
| 34 | SEQ ID NO: 93 |
| 35 | SEQ ID NO: 94 |
| 36 | SEQ ID NO: 95 |
| 37 | SEQ ID NO: 96 |
| 38 | SEQ ID NO: 97 |
| 39 | SEQ ID NO: 98 |
| 40 | SEQ ID NO: 99 |
| 41 | SEQ ID NO: 100 |
| 42 | SEQ ID NO: 101 |
| 43 | SEQ ID NO: 102 |
| 44 | SEQ ID NO: 103 |
| 45 | SEQ ID NO: 104 |
| 46 | SEQ ID NO: 105 |
| 47 | SEQ ID NO: 106 |
| 48 | SEQ ID NO: 107 |
| 49 | SEQ ID NO: 108 |
| 50 | SEQ ID NO: 109 |
| 51 | SEQ ID NO: 110 |
| 52 | SEQ ID NO: 111 |
| 53 | SEQ ID NO: 112 |

As used herein, the term “subject” includes any mammal in need of these methods of treatment or prophylaxis, including humans. Other mammals in need of such treatment or prophylaxis include dogs, cats, or other domesticated animals, horses, livestock, laboratory animals, including non-human primates, etc. The subject may be male or female. In one embodiment, the subject has a disease or disorder caused by a mutation in the ABCA4 gene (e.g., Stargardt Disease, e.g., Stargardt Disease 1) or the CEP290 gene (e.g., an autosomal recessive disorder, such as LCA 10). In another embodiment, the subject is at risk of developing a disease or disorder caused by a mutation in the ABCA4 gene or the CEP290 gene. In another embodiment, the subject has shown clinical signs of a disease or disorder caused by a mutation in the ABCA4 gene (such as Stargardt Disease) or the CEP290 gene (such as LCA 10). The subject may be any age during which treatment or prophylactic therapy may be beneficial. For example, in some embodiments, the subject is 0-5 years of age, 5-10 years of age, 10-20 years of age, 20-30 years of age, 30-50 years of age, 50-70 years of age, or more than 70 years of age. In another embodiment, the subject is 12 months of age or older, 18 months of age or older, 2 years of age or older, 3 years of age or older, 4 years of age or older, 5 years of age or older, 6 years of age or older, 7 years of age or older, 8 years of age or older, 9 years of age or older, or 10 years of age or older. In another embodiment, the subject has viable retinal cells.

As used herein, the terms “disorder associated with a mutation” or “mutation associated with a disorder” refer to a correlation between a disorder and a mutation. In some embodiments, a disorder associated with a mutation is known or suspected to be wholly or partially, or directly or indirectly, caused by the mutation. For example, a subject having the mutation may be at risk of developing the disorder, and the risk may additionally depend on other factors, such as other (e.g., independent) mutations (e.g., in the same or a different gone), or environmental factors.

As used herein, the term “treatment,” or a grammatical derivation thereof, is defined as reducing the progression of a disease, reducing the severity of a disease symptom, retarding progression of a disease symptom, removing a disease symptom, or delaying onset of a disease.

As used herein, the term “prevention” of a disorder, or a grammatical derivation thereof, is defined as reducing the risk of onset of a disease, e.g., as a prophylactic therapy for a subject who is at risk of developing a disorder associated with a mutation. A subject can be characterized as “at risk” of developing a disorder by identifying a mutation associated with the disorder, according to any suitable method known in the art or described herein. In some embodiment, a subject who is at risk of developing a disorder has one or more ABCA4 or CEP290 mutations associated with the disorder. Additionally or alternatively, a subject can be characterized as “at risk” of developing a disorder if the subject has a family history of the disorder.

Treating or preventing a disorder in a subject can be performed by directly administering the trans-splicing molecule (e.g., within an AAV vector or AAV particle) to the subject. Alternatively, host cells containing the trans-splicing molecule may be administered to the subject.

The term “administering,” or a grammatical derivation thereof, as used in the methods described herein, refers to delivering the composition, or an ex vivo-treated cell, to the subject in need thereof, e.g., having a mutation or defect in the targeted gene. For example, in one embodiment in which ocular cells are targeted, the method involves delivering the composition by subretinal injection to the photoreceptor cells or other ocular cells. In another embodiment, intravitreal injection to ocular cells or injection via the palpebral vein to ocular cells may be employed. In another embodiment, the composition is administered intravenously. Still other methods of administration may be selected by one of skill in the art, in view of this disclosure.

Codon optimization refers to modifying a nucleic acid sequence to change individual nucleic acids without any resulting change in the encoded amino acid. Sequences modified in this way are referred to herein as “codon-optimized.” This process may be performed on any of the sequences described in this specification to enhance expression or stability. Codon optimization may be performed in a manner such as that described in, e.g., U.S. Pat. Nos. 7,561,972, 7,561,973, and 7,888,112, each of which is incorporated herein by reference in its entirety. The sequence surrounding the translational start site can be converted to a consensus Kozak sequence according to known methods. See, e.g., Kozak et al, Nucleic Acids Res. 15 (20): 8125-8148, incorporated herein by reference in its entirety.

The term “homologous” refers to the degree of identity between sequences of two nucleic acid sequences. The homology of homologous sequences is determined by comparing two sequences aligned under optimal conditions over the sequences to be compared. The sequences to be compared herein may have an addition or deletion (for example, gap and the like) in the optimum alignment of the two sequences. Such a sequence homology can be calculated by creating an alignment using, for example, the ClustalW algorithm (Nucleic Acid Res., 1994, 22 (22): 4673 4680). Commonly available sequence analysis software, such as, Vector NTI, GENETYX, BLAST, or analysis tools provided by public databases may also be used.

The term “pharmaceutically acceptable” means safe for administration to a mammal, such as a human. In some embodiments, a pharmaceutically acceptable composition is approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which a therapeutic molecule (e.g., a trans-splicing molecule or a trans-splicing molecule including a vector or cell of the present invention) is administered. Examples of suitable pharmaceutical carriers are described in “Remington's Pharmaceutical Sciences,” Mack Publishing Co., Easton, PA., 2^{nd} edition, 2005.

The terms “a” and “an” mean “one or more of.” For example, “a gene” is understood to represent one or more such genes. As such, the terms “a” and “an,” “one or more of a (or an),” and “at least one of a (or an)” are used interchangeably herein.

As used herein, the term “about” refers to a value within ±10% variability from the reference value, unless otherwise specified.

### II. Trans-Splicing Molecules

Provided herein are ABCA4 trans-splicing molecules and CEP290 trans-splicing molecules.

### ABCA4 Trans-Splicing Molecules

The present invention features nucleic acid trans-splicing molecules useful for treating diseases and disorders associated with a mutation in an ABCA4 gene by replacing one or more exons in the ABCA4 gene (e.g., an ABCA4 gone having a mutated ABCA4 exon). In some embodiments, the nucleic acid trans-splicing molecule is a pre-RNA trans-splicing molecule (RTM). The design of the trans-splicing molecule permits replacement of the defective or mutated portion of the pre-mRNA exon(s) with a nucleic acid sequence, e.g., the exon(s) having a functional (e.g., normal) sequence without the mutation. The functional sequence can be a wild-type, naturally-occurring sequence or a corrected sequence with some other modification, e.g., codon optimization.

In one embodiment, a trans-splicing molecule is configured to correct one or more mutations located on a 3′ portion of the ABCA4 gene. In one embodiment, a trans-splicing molecule is configured to correct one or more mutations located on a 5′ portion of the ABCA4 gene. The trans-splicing molecules provided herein function to repair the defective gene in the target cell of a subject by replacing the defective pre-mRNA gene sequence and removing the defective portion of the target pre-mRNA, yielding a functional ABCA4 gene capable of transcribing a functional gene product in the cell.

The invention provides trans-splicing molecules having a binding domain configured to bind a target ABCA4 intron, a splicing domain configured to mediate trans-splicing, and a coding domain having one or more functional ABCA4 exons. In a 5′ trans-splicing molecule, the coding domain, splice site, and binding domain are operatively linked in a 5′-to-3′ direction, such that the trans-splicing molecule is configured to replace the 5′ end of the endogenous gene with the coding domain, which includes a functional ABCA4 exon to replace the mutated ABCA4 exon. Conversely, in a 3′ trans-splicing molecule, the coding domain, splice site, and binding domain are operatively linked in a 3′-to-5′ direction, such that the trans-splicing molecule is configured to replace the 3′ end of the endogenous gene with the coding domain, which includes a functional ABCA4 exon to replace the mutated ABCA4 exon. In some embodiments, the splicing domain resides within an artificial intron, which links the binding domain to the coding domain. The artificial intron may include additional components, such as a spacer.

In some embodiments, the trans-splicing molecule or coding domain thereof is up to 4,700 nucleotide bases in length (e.g., from 200 to 300 nucleotide bases in length, from 300 to 400 nucleotide bases in length, from 400 to 500 nucleotide bases in length, from 500 to 600 nucleotide bases in length, from 600 to 700 nucleotide bases in length, from 700 to 800 nucleotide bases in length, form 800 to 900 nucleotide bases in length, from 900 to 1,000 nucleotide bases in length, from 1,000 to 1,500 nucleotide bases in length, from 1,500 to 2,000 nucleotide bases in length, from 2,000 to 2,500 nucleotide bases in length, from 2,500 to 3,000 nucleotide bases in length, or from 3,000 to 4,000 nucleotide bases in length, e.g., from 3,100 to 3,800 nucleotide bases in length, from 3,200 to 3,700 nucleotide bases in length, or from 3,300 to 3,500 nucleotide bases in length, e.g., from 3,000 to 3,100 nucleotide bases in length, from 3,100 to 3,200 nucleotide bases in length, from 3,200 to 3,300 nucleotide bases in length, from 3,300 to 3,400 nucleotide bases in length, from 3,400 to 3,500 nucleotide bases in length, from 3,500 to 3,600 nucleotide bases in length, from 3,600 to 3,700 nucleotide bases in length, from 3,700 to 3,800 nucleotide bases in length, from 3,800 to 3,900 nucleotide bases in length, or from 3,900 to 4,000 nucleotide bases in length, e.g., about 2,918 nucleotide bases in length, about 3,328 nucleotide bases in length, about 3,522 nucleotide bases in length, about 3,607 nucleotide bases in length, about 3,632 nucleotide bases in length, about 3,494 nucleotide bases in length, or about 3,300 nucleotide bases in length).

Due to the large size of the ABCA4 gene and the size constraints of AAV delivery, a single trans-splicing molecule configured for packaging within an AAV vector may not span all mutations in an ABCA4 gene that may be associated with a disorder and thereby may not correct mutations along the length of the entire ABCA4 gene. Accordingly, the trans-splicing molecules of the invention can be adapted as part of methods described below to correct multiple mutations spanning the entire length of the ABCA4 gene.

An ABCA4 gene targeted by a trans-splicing molecule described herein contains one or multiple mutations that are associated with (e.g., cause, or are correlated with) a disease, such as a Stargardt Disease (e.g., Stargardt Disease 1). An exemplary DNA sequence of a functional (wildtype) human ABCA4 gene is given by the NCBI Reference Sequence: NG_009073. The amino acid sequence of an exemplary protein retinal-specific ATP-binding cassette transporter expressed by ABCA4 is given by Protein Accession No. P78363.

In addition to these published sequences, all corrections later obtained or naturally occurring conservative and non-disease-causing variants sequences that occur in the human or other mammalian population are also included. Additional conservative nucleotide replacements or those causing codon optimizations are also included. The sequences as provided by the database accession numbers may also be used to search for homologous sequences in the same or another mammalian organism.

It is anticipated that the ABCA4 nucleic acid sequences and resulting protein truncates or amino acid fragments may tolerate certain minor modifications at the nucleic acid level to include, for example, modifications to the nucleotide bases which are silent, e.g., preference codons. In other embodiments, nucleic acid base modifications which change the amino acids, e.g., to improve expression of the resulting peptide/protein (for example, codon optimization) are anticipated. Also included as likely modification of fragments are allelic variations, caused by the natural degeneracy of the genetic code.

Also included as modifications of ABCA4 genes are analogs, or modified versions, of the encoded protein fragments provided herein. Typically, such analogs differ from the specifically identified proteins by only one to four codon changes. Conservative replacements are those that take place within a family of amino acids that are related in their side chains and chemical properties.

The nucleic acid sequence of a functional ABCA4 gene may be derived from any mammal which natively expresses functional retinal-specific ATP-binding cassette transporter, or homolog thereof. In other embodiments, certain modifications are made to the ABCA4 gene sequence in order to enhance expression in the target cell. Such modifications include codon optimization.

In some embodiments, the disorder associated with a mutation in ABCA4 is an autosomal recessive disease, for example, Stargardt Disease. In certain instances involving a subject having an autosomal recessive disorder, the subject has a mutation in ABCA4 on both alleles. Compositions comprising trans-splicing molecules can correct the mutations on both alleles, regardless of the location of the mutation within the ABCA4 gene. For instance, for a subject having a mutated ABCA4 exon 1 on a first allele and a mutated ABCA4 exon 30 on a second allele, provided herein is a composition having a 5′ trans-splicing molecule to replace the mutated ABCA4 exon 1 and a 3′ trans-splicing molecule to replace the mutated ABCA4 exon 30. In such embodiments, the two trans-splicing molecules can be co-delivered as part of the same AAV vector or delivered in separate AAV vectors (e.g., in the case in which both trans-splicing molecules exceed the packaging limit of AAV).

Alternatively, in embodiments in which two or more mutations are located on a portion of the ABCA4 gene that can be replaced by the same trans-splicing molecule, a single trans-splicing molecule having a coding region containing a functional ABCA4 exon can replace the one or more exons containing the mutations. Mutations in particular ABCA4 exons are also listed in International Patent Publication No. WO 2017/087900, incorporated herein by reference.

### ABCA4 Coding Domains

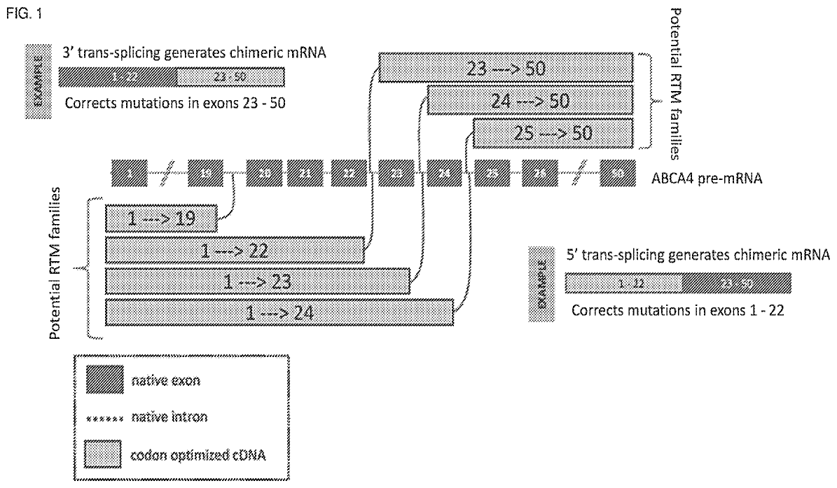

In some embodiments, the coding domain of a 5′ trans-splicing molecule includes all ABCA4 exons (e.g., functional ABCA4 exons) that are 5′ to the target ABCA4 intron. For example, in embodiments in which a 5′ trans-splicing molecule targets ABCA4 intron 19, the coding domain includes functional ABCA4 exons 1-19. In such embodiments featuring a 5′ trans-splicing molecule having a coding domain including functional ABCA4 exons 1-19, the coding domain is about 2918 bp in length. In embodiments in which a 5′ trans-splicing molecule targets ABCA4 intron 22, the coding domain includes functional ABCA4 exons 1-22. In such embodiments featuring a 5′ trans-splicing molecule having a coding domain including functional ABCA4 exons 1-22, the coding domain is about 3,328 bp in length. In embodiments in which a 5′ trans-splicing molecule targets ABCA4 intron 23, the coding domain includes functional ABCA4 exons 1-23. In such embodiments featuring a 5′ trans-splicing molecule having a coding domain including functional ABCA4 exons 1-23, the coding domain is about 3,522 bp in length. In embodiments in which a 5′ trans-splicing molecule targets ABCA4 intron 24, the coding domain includes functional ABCA4 exons 1-24. In such embodiments featuring a 5′ trans-splicing molecule having a coding domain including functional ABCA4 exons 1-24, the coding domain is about 3,607 bp in length. The aforementioned embodiments of 5′ ABCA4-targeting trans-splicing molecules are illustrated at the lower left-hand portion of FIG. 1.

In some embodiments, the coding domain of a 3′ trans-splicing molecule includes any one or more of ABCA4 exons 20-50. For example, in embodiments in which a 3′ trans-splicing molecule targets ABCA4 intron 22, the coding domain includes functional ABCA4 exons 23-50. In such embodiments featuring a 3′ trans-splicing molecule having a coding domain including functional ABCA4 exons 23-50, the coding domain is about 3,632 bp in length. In embodiments in which a 3′ trans-splicing molecule targets ABCA4 intron 23, the coding domain includes functional ABCA4 exons 24-50. In such embodiments featuring a 3′ trans-splicing molecule having a coding domain including functional ABCA4 exons 24-50, the coding domain is about 3,494 bp in length. In embodiments in which a 3′ trans-splicing molecule targets ABCA4 intron 24, the coding domain includes functional ABCA4 exons 25-50. In such embodiments featuring a 3′ trans-splicing molecule having a coding domain including functional ABCA4 exons 25-50, the coding domain is about 3,300 bp in length. The aforementioned embodiments of 3′ ABCA4-targeting trans-splicing molecules are illustrated at the upper right-hand portion of FIG. 1.

In some embodiments, the coding domain includes 1, 2, 3, 4, 5, 6, 7, 8, 9, 10, 11, 12, 13, 14, 15, 16, 17, 18, 19, 20, 21, 22, 23, 24, 25, 26, 27, or 28 functional ABCA4 exons.

In some instances, both mutations occur in the 5′ portion of the target gene, and a 5′ trans-splicing molecule is selected to correct both mutations. In one embodiment, the binding domain binds to intron 19, and the coding domain includes functional ABCA4 exons 1-19. In one embodiment, the binding domain binds to intron 22, and the coding domain includes functional ABCA4 exons 1-22. In one embodiment, the binding domain binds to intron 23, and the coding domain includes functional ABCA4 exons 1-23. In one embodiment, the binding domain binds to intron 24, and the coding domain includes functional ABCA4 exons 1-24. Alternatively, in instances in which both mutations occur on the 3′ portion of the target gene, a 3′ trans-splicing molecule is selected to correct both mutations. In one embodiment, the binding domain binds to intron 22, and the coding domain includes functional ABCA4 exons 23-50. In one embodiment, the binding domain binds to intron 23, and the coding domain includes functional ABCA4 exons 24-50. In one embodiment, the binding domain binds to intron 24, and the coding domain includes functional ABCA4 exons 25-50.

As one example, a 3′ pre-mRNA ABCA4 trans-splicing molecule operates as follows: A chimeric mRNA is created through a trans-splicing reaction mediated by the spliceosome between the 5′ splice site of the endogenous target pre-mRNA and the 3′ splice site of the trans-splicing molecule. The trans-splicing molecule binds through specific base pairing to a target ABCA4 intron of the endogenous target pre-mRNA and replaces the whole 3′ sequence of the endogenous ABCA4 gene upstream of the targeted intron with the coding domain having a functional ABCA4 exon sequence of the trans-splicing molecule.

A 3′ trans-splicing molecule includes a binding domain which binds to the target ABCA4 intron 5′ to the mutation or defect, an artificial intron comprising optional spacer and a 3′ splice site, and a coding domain that encodes all exons of the ocular target gene that are 3′ to the binding of the binding domain to the target. A 5′ trans-splicing molecule includes a binding domain binds to the target ABCA4 intron 3′ to the mutation or defect, a 5′ splice site, an optional spacer and a coding domain that encodes all exons of the ocular target gene that are 5′ to the binding of the binding domain to the target.

In some embodiments, the coding domain includes a complementary DNA (cDNA) sequence. For example, one or more functional ABCA4 exons within the coding domain can be a cDNA sequence. In some embodiments, the entire coding domain is a cDNA sequence. Additionally or alternatively, all or a portion of the coding domain, or one or more functional ABCA4 exons thereof, can be a naturally-occurring sequence (e.g., a sequence having 90%, 91%, 92%, 93%, 94%, 95%, 96%, 97%, 98%, 99%, or 100% sequence identity with an endogenous ABCA4 exon).

In some embodiments, all or a portion of the coding domain, or one or more functional ABCA4 exons thereof, is a codon-optimized sequence in which a nucleic acid sequence has been modified, e.g., to enhance expression or stability, without resulting in a change in the encoded amino acid. Codon optimization may be performed in a manner such as that described in, e.g., U.S. Pat. Nos. 7,561,972, 7,561,973, and 7,888,112, each of which is incorporated herein by reference in its entirety. For delivery via a recombinant AAV, as described herein, in one embodiment, the coding domain can be a nucleic acid sequence of up to 4,000 nucleotide bases in length (e.g., from 3,000 to 4,000 nucleotide bases in length, from 3,100 to 3,800 nucleotide bases in length, from 3,200 to 3,700 nucleotide bases in length, or from 3,300 to 3,500 nucleotide bases in length, e.g., from 3,000 to 3,100 nucleotide bases in length, from 3,100 to 3,200 nucleotide bases in length, from 3,200 to 3,300 nucleotide bases in length, from 3,300 to 3,400 nucleotide bases in length, from 3,400 to 3,500 nucleotide bases in length, from 3,500 to 3,600 nucleotide bases in length, from 3,600 to 3,700 nucleotide bases in length, from 3,700 to 3,800 nucleotide bases in length, from 3,800 to 3,900 nucleotide bases in length, or from 3,900 to 4,000 nucleotide bases in length).

### ABCA4 Binding Domains

Trans-splicing molecules of the invention feature a binding domain configured to bind a target ABCA4 intron. In one embodiment, the binding domain is a nucleic acid sequence complementary to a sequence of the target ABCA4 pre-mRNA (e.g., a target ABCA4 intron) to suppress endogenous target cis-splicing while enhancing trans-splicing between the trans-spicing molecule and the target ABCA4 pre-mRNA, e.g., to create a chimeric molecule having a portion of endogenous ABCA4 mRNA and the coding domain having one or more functional ABCA4 exons. In some embodiments, the binding domain is in an antisense orientation to a sequence of the target ABCA4 intron.

A 5′ trans-splicing molecule will generally bind the target ABCA4 intron 3′ to the mutation, while a 3′ trans-splicing molecule will generally bind the target ABCA4 intron 5′ to the mutation. In one embodiment, the binding domain comprises a part of a sequence complementary to the target ABCA4 intron. In one embodiment herein, the binding domain is a nucleic acid sequence complementary to the intron closest to (i.e., adjacent to) the exon sequence that is being corrected.

In another embodiment, the binding domain is targeted to an intron sequence in close proximity to the 3′ or 5′ splice signals of a target intron. In still another embodiment, a binding domain sequence can bind to the target intron in addition to part of an adjacent exon.

Thus, in some instances, the binding domain binds specifically to the mutated endogenous target pre-mRNA to anchor the coding domain of the trans-splicing molecule to the pre-mRNA to permit trans-splicing to occur at the correct position in the target ABCA4 gene. The spliceosome processing machinery of the nucleus may then mediate successful trans-splicing of the corrected exon for the mutated exon causing the disease.

In certain embodiments, the trans-splicing molecules feature binding domains that contain sequences on the target pre-mRNA that bind in more than one place. The binding domain may contain any number of nucleotides necessary to stably bind to the target pro-mRNA to permit trans-splicing to occur with the coding domain. In one embodiment, the binding domains are selected using mFOLD structural analysis for accessible loops (Zuker, Nucleic Acids Res. 2003, 31 (13): 3406-3415).

Suitable target binding domains can be from 10 to 500 nucleotides in length. In some embodiments, the binding domain is from 20 to 400 nucleotides in length. In some embodiments, the binding domain is from 50 to 300 nucleotides in length. In some embodiments, the binding domain is from 100 to 200 nucleotides in length. In some embodiments, the binding domain is from 10-20 nucleotides in length (e.g., 10, 11, 12, 13, 14, 15, 16, 17, 18, 19, or 20 nucleotides in length), 20-30 nucleotides in length (e.g., 20, 21, 22, 23, 24, 25, 26, 27, 28, 29, or 30 nucleotides in length), 30-40 nucleotides in length (e.g., 30, 31, 32, 33, 34, 35, 36, 37, 38, 39, or 40 nucleotides in length), 40-50 nucleotides in length (e.g., 40, 41, 42, 43, 44, 45, 46, 47, 48, 49, 50 nucleotides in length), 50-60 nucleotides in length (e.g., 50, 51, 52, 53, 54, 55, 56, 57, 58, 59, or 60 nucleotides in length), 60-70 nucleotides in length (e.g., 60, 61, 62, 63, 64, 65, 66, 67, 68, 69, or 70 nucleotides in length), 70-80 nucleotides in length (e.g., 70, 71, 72, 73, 74, 75, 76, 77, 78, 79, or 80 nucleotides in length), 80-90 nucleotides in length (e.g., 80, 81, 82, 83, 84, 85, 86, 87, 88, 89, or 90 nucleotides in length), 90-100 nucleotides in length (e.g., 90, 91, 92, 93, 94, 95, 96, 97, 98, 99, or 100 nucleotides in length), 100-110 nucleotides in length (e.g., 100, 101, 102, 103, 104, 105, 106, 107, 108, 109, or 110 nucleotides in length), 110-120 nucleotides in length (e.g., 110, 111, 112, 113, 114, 115, 116, 117, 118, 119, or 120 nucleotides in length), 120-130 nucleotides in length (e.g., 120, 121, 122, 123, 124, 125, 126, 127, 128, 129, or 130 nucleotides in length), 130-140 nucleotides in length (e.g., 130, 131, 132, 133, 134, 135, 136, 137, 138, 139, or 140 nucleotides in length), 140-150 nucleotides in length (e.g., 140, 141, 142, 143, 144, 145, 146, 147, 148, 149, or 150 nucleotides in length), 150-160 nucleotides in length (e.g., 150, 151, 152, 153, 154, 155, 156, 157, 158, 159, or 160 nucleotides in length), 160-170 nucleotides in length (e.g., 160, 161, 162, 163, 164, 165, 166, 167, 168, 169, or 170 nucleotides in length), 170-180 nucleotides in length (e.g., 170, 171, 172, 173, 174, 175, 176, 177, 178, 179, or 180 nucleotides in length), 180-190 nucleotides in length (e.g., 180, 181, 182, 183, 184, 185, 186, 187, 188, 189, or 190 nucleotides in length), 190-200 nucleotides in length (e.g., 190, 191, 192, 193, 194, 195, 196, 197, 198, 199, or 200 nucleotides in length), 200-210 nucleotides in length, 210-220 nucleotides in length, 220-230 nucleotides in length, 230-240 nucleotides in length, 240-250 nucleotides in length, 250-260 nucleotides in length, 260-270 nucleotides in length, 270-280 nucleotides in length, 280-290 nucleotides in length, 290-300 nucleotides in length, 300-350 nucleotides in length, 350-400 nucleotides in length, 400-450 nucleotides in length, or 450-500 nucleotides in length. In some embodiments, the binding domain is about 150 nucleotides in length. In another embodiment, the target binding domains may include a nucleic acid sequence up to 750 nucleotides in length. In another embodiment, the target binding domains may include a nucleic acid sequence up to 1000 nucleotides in length. In another embodiment, the target binding domains may include a nucleic acid sequence up to 2000 nucleotides or more in length.

In some embodiments, the specificity of the trans-splicing molecule may be increased by increasing the length of the target binding domain. Other lengths may be used depending upon the lengths of the other components of the trans-splicing molecule.

The binding domain may be from 80% to 100% complementary to the target intron to be able to hybridize stably with the target intron. For example, in some embodiments, the binding domain is 80%, 81%, 82%, 83%, 84%, 85%, 86%, 87%, 88%, 89%, 90%, 91%, 92%, 93%, 94%, 95%, 96%, 97%, 98%, 99%, or 100% complimentary to the target intron. The degree of complementarity is selected by one of skill in the art based on the need to keep the trans-splicing molecule and the nucleic acid construct containing the necessary sequences for expression and for inclusion in the rAAV within a 3,000 or up to 4,000 nucleotide base limit. The selection of this sequence and strength of hybridization depends on the complementarity and the length of the nucleic acid.

Any of the aforementioned binding domains may bind to a binding site within intron 19 (SEQ ID NO: 25), intron 22 (SEQ ID NO: 28) intron 23 (SEQ ID NO: 29), or intron 24 (SEQ ID NO: 30).

In certain instances of the invention, the trans-splicing molecule is a 5′ trans-splicing molecule and features a binding domain that binds to intron 19 of ABCA4 (SEQ ID NO: 25) and includes a coding domain having functional ABCA4 exons 1-19. In some embodiments, the binding site comprises any one or more of nucleotides 990 to 2,174 of SEQ ID NO: 25 (e.g., any one or more of nucleotides 1,670 to 2,174 of SEQ ID NO: 25, any one or more of nucleotides 1,810 to 2,000 of SEQ ID NO: 25, any one or more of nucleotides 1,870 to 2,000 of SEQ ID NO: 25, or any one or more of nucleotides 1,920 to 2,000 of SEQ ID NO: 25).

In some embodiments, the trans-splicing molecule is a 5′ trans-splicing molecule and features a binding domain that binds to intron 22 of ABCA4 (SEQ ID NO: 28) and includes a coding domain having functional ABCA4 exons 1-22. In some embodiments, the binding site comprises any one or more of nucleotides 60 to 570, nucleotides 600 to 800, or nucleotides 900 to 1,350 of SEQ ID NO: 28 (e.g., any one or more of nucleotides 70 to 250 of SEQ ID NO: 28).

Alternatively, the trans-splicing molecule can be a 3′ trans-splicing molecule and can feature a binding domain that binds to intron 22 of ABCA4 (SEQ ID NO: 28). This trans-splicing molecules may include a coding domain having functional ABCA4 exons 23-50. In some embodiments, the binding site comprises any one or more of nucleotides 1 to 510 or 880 to 1,350 of SEQ ID NO: 28.

In other embodiments, the trans-splicing molecule is a 5′ trans-splicing molecule and features a binding domain that binds to intron 23 of ABCA4 (SEQ ID NO: 29) and includes a coding domain having functional ABCA4 exons 1-23. In some embodiments, the binding site comprises any one or more of nucleotides 80 to 570 or 720 to 1,081 of SEQ ID NO: 29.

Alternatively, the trans-splicing molecule can be a 3′ trans-splicing molecule and can feature a binding domain that binds to intron 23 of ABCA4 (SEQ ID NO: 29) and a coding domain having functional ABCA4 exons 24-50. In some embodiments, the binding site comprises any one or more of nucleotides 80 to 1,081 of SEQ ID NO: 29 (e.g., any one or more of nucleotides 230 to 1,081 of SEQ ID NO: 29, any one or more of nucleotides 250 to 400 of SEQ ID NO: 29, or any one or more of nucleotides 690 to 850 of SEQ ID NO: 29).

In some embodiments, the trans-splicing molecule is a 5′ trans-splicing molecule and features a binding domain that binds to intron 24 of ABCA4 (SEQ ID NO: 30) and includes a coding domain having functional ABCA4 exons 1-24. In some embodiments, the binding site comprises any one or more of nucleotides 600 to 1,250 or 1,490 to 2,660 of SEQ ID NO: 30 (e.g., any one or more of nucleotides 1,000 to 1,200 of SEQ ID NO: 30).

In other embodiments, the trans-splicing molecule is a 3′ trans-splicing molecule and features a binding domain that binds to intron 24 of ABCA4 (SEQ ID NO: 30) and includes a coding domain having functional ABCA4 exons 25-50. In some embodiments, the binding site comprises any one or more of nucleotides 1 to 250, nucleotides 300 to 2,100, or nucleotides 2,200 to 2,692 of SEQ ID NO: 30 (e.g., any one or more of nucleotides 360 to 610 of SEQ ID NO: 30 or any one or more of nucleotides 750 to 1,110 of SEQ ID NO: 30).

### CEP290 Trans-Splicing Molecules

The present invention features nucleic acid trans-splicing molecules useful for treating diseases and disorders associated with a mutation in a CEP290 gene by replacing one or more exons in the CEP290 gene (e.g., a CEP290 gene having a mutation in intron 26). In some embodiments, the nucleic acid trans-splicing molecule is a pre-RNA trans-splicing molecule (RTM). The design of the trans-splicing molecule permits replacement of the defective or mutated portion of the pre-mRNA with a nucleic acid sequence, e.g., the exon(s) having a functional (e.g., normal) sequence without the mutation. The functional sequence can be a wild-type, naturally-occurring sequence or a corrected sequence with some other modification, e.g., codon optimization.

In one embodiment, a trans-splicing molecule is configured to correct one or more mutations located on a 5′ portion of the CEP290 gene. The trans-splicing molecules provided herein function to repair the defective gene in the target cell of a subject by replacing the defective pre-mRNA gene sequence, yielding a functional CEP290 gone capable of transcribing a functional gene product in the cell.

The invention provides trans-splicing molecules having a binding domain configured to bind a target CEP290 intron, a splicing domain configured to mediate trans-splicing, and a coding domain having one or more functional CEP290 exons. In a 5′ trans-splicing molecule, the coding domain, splice site, and binding domain are operatively linked in a 5′-to-3′ direction, such that the trans-splicing molecule is configured to replace the 5′ end of the endogenous gene with the coding domain, which includes a functional CEP290 exon to corrected the mutated CEP290 pre-mRNA. In some embodiments, the splicing domain resides within an artificial intron, which links the binding domain to the coding domain. The artificial intron may include additional components, such as a spacer.

In some embodiments, the trans-splicing molecule is up to 4,700 nucleotide bases in length (e.g., from 3,000 to 4,000 nucleotide bases in length, from 3,100 to 3,800 nucleotide bases in length, from 3,200 to 3,700 nucleotide bases in length, or from 3,300 to 3,500 nucleotide bases in length, e.g., from 3,000 to 3,100 nucleotide bases in length, from 3,100 to 3,200 nucleotide bases in length, from 3,200 to 3,300 nucleotide bases in length, from 3,300 to 3,400 nucleotide bases in length, from 3,400 to 3,500 nucleotide bases in length, from 3,500 to 3,600 nucleotide bases in length, from 3,600 to 3,700 nucleotide bases in length, from 3,700 to 3,800 nucleotide bases in length, from 3,800 to 3,900 nucleotide bases in length, or from 3,900 to 4,000 nucleotide bases in length, e.g., about 2,991 nucleotide bases in length, about 3,103 nucleotide bases in length, about 3,309 nucleotide bases in length, about 3,461 nucleotide bases in length, or about 3,573 nucleotide bases in length).

A CEP290 gene targeted by a trans-splicing molecule described herein contains one or multiple mutations that are associated with (e.g., cause, or are correlated with) a disease, such as Leber congenital amourosis (e.g., LCA 10). An exemplary DNA sequence of a functional (wildtype) human CEP290 gene is given by the NCBI Reference Sequence: NG_008417. The amino acid sequence of an exemplary centrosomal protein 290 is given by Protein Accession No. O15078.

In addition to these published sequences, all corrections later obtained or naturally occurring conservative and non-disease-causing variants sequences that occur in the human or other mammalian population are also included. Additional conservative nucleotide replacements or those causing codon optimizations are also included. The sequences as provided by the database accession numbers may also be used to search for homologous sequences in the same or another mammalian organism.

It is anticipated that the CEP290 nucleic acid sequences and resulting protein truncates or amino acid fragments may tolerate certain minor modifications at the nucleic acid level to include, for example, modifications to the nucleotide bases which are silent, e.g., preference codons. In other embodiments, nucleic acid base modifications which change the amino acids, e.g., to improve expression of the resulting peptide/protein (for example, codon optimization) are anticipated. Also included as likely modification of fragments are allelic variations, caused by the natural degeneracy of the genetic code.

Also included as modifications of CEP290 genes are analogs, or modified versions, of the encoded protein fragments provided herein. Typically, such analogs differ from the specifically identified proteins by only one to four codon changes. Conservative replacements are those that take place within a family of amino acids that are related in their side chains and chemical properties.

The nucleic acid sequence of a functional CEP290 gone may be derived from any mammal which natively expresses functional centrosomal protein 290, or homolog thereof. In other embodiments, certain modifications are made to the CEP290 gene sequence in order to enhance expression in the target cell. Such modifications include codon optimization.

CEP290 mutations can be found on the CCHMC Molecular Genetics Laboratory Mutation Database, LOVD v.2.0. Mutations in particular CEP290 exons are also listed in International Patent Publication No. WO 2017/087900, incorporated herein by reference. Table 3, above, provides information regarding the size and position of each exon and intron of CEP290.

In some embodiments, the disorder associated with a mutation in CEP290 is an autosomal recessive disease, for example, LCA 10.

### Coding Domains

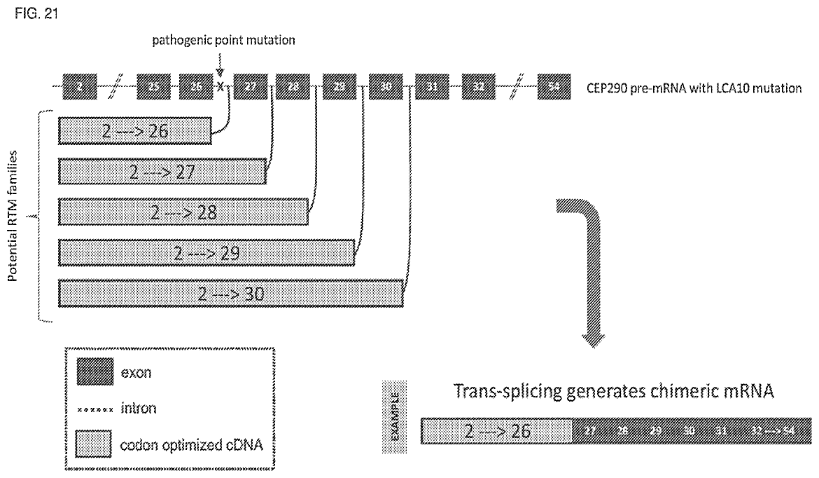

In some embodiments, the coding domain of a 5′ trans-splicing molecule includes all CEP290 exons (e.g., functional CEP290 exons) that are 5′ to the target CEP290 intron. For example, in embodiments in which a 5′ trans-splicing molecule targets CEP290 intron 26, the coding domain includes functional CEP290 exons 2-26. In such embodiments featuring a 5′ trans-splicing molecule having a coding domain including functional CEP290 exons 2-26, the coding domain is about 2,991 bp in length. In embodiments in which a 5′ trans-splicing molecule targets CEP290 intron 27, the coding domain includes functional CEP290 exons 2-27. In such embodiments featuring a 5′ trans-splicing molecule having a coding domain including functional CEP290 exons 2-27, the coding domain is about 3, 103 bp in length. In embodiments in which a 5′ trans-splicing molecule targets CEP290 intron 28, the coding domain includes functional CEP290 exons 2-28. In such embodiments featuring a 5′ trans-splicing molecule having a coding domain including functional CEP290 exons 2-28, the coding domain is about 3,309 bp in length. In embodiments in which a 5′ trans-splicing molecule targets CEP290 intron 29, the coding domain includes functional CEP290 exons 2-29. In such embodiments featuring a 5′ trans-splicing molecule having a coding domain including functional CEP290 exons 2-29, the coding domain is about 3,461 bp in length. In embodiments in which a 5′ trans-splicing molecule targets CEP290 intron 30, the coding domain includes functional CEP290 exons 2-30. In such embodiments featuring a 5′ trans-splicing molecule having a coding domain including functional CEP290 exons 2-30, the coding domain is about 3,573 bp in length. The aforementioned embodiments of 5′ CEP290-targeting trans-splicing molecules are illustrated in FIG. 21.

In some embodiments, the coding domain includes 25, 26, 27, 28, or 29 functional CEP290 exons.

In some embodiments, the coding domain includes a complementary DNA (cDNA) sequence. For example, one or more functional CEP290 exons within the coding domain can be a cDNA sequence. In some embodiments, the entire coding domain is a cDNA sequence. Additionally or alternatively, all or a portion of the coding domain, or one or more functional CEP290 exons thereof, can be a naturally-occurring sequence (e.g., a sequence having 90%, 91%, 92%, 93%, 94%, 95%, 96%, 97%, 98%, 99%, or 100% sequence identity with an endogenous CEP290 exon).

In some embodiments, all or a portion of the coding domain, or one or more functional CEP290 exons thereof, is a codon-optimized sequence in which a nucleic acid sequence has been modified, e.g., to enhance expression or stability, without resulting in a change in the encoded amino acid. Codon optimization may be performed in a manner such as that described in, e.g., U.S. Patent Nos. 7,561,972, 7,561,973, and 7,888,112, each of which is incorporated herein by reference in its entirety. For delivery via a recombinant AAV, as described herein, in one embodiment, the coding domain can be a nucleic acid sequence of up to 4,000 nucleotide bases in length (e.g., from 3,000 to 4,000 nucleotide bases in length, from 3,100 to 3,800 nucleotide bases in length, from 3,200 to 3,700 nucleotide bases in length, or from 3,300 to 3,500 nucleotide bases in length, e.g., from 3,000 to 3,100 nucleotide bases in length, from 3,100 to 3,200 nucleotide bases in length, from 3,200 to 3,300 nucleotide bases in length, from 3,300 to 3,400 nucleotide bases in length, from 3,400 to 3,500 nucleotide bases in length, from 3,500 to 3,600 nucleotide bases in length, from 3,600 to 3,700 nucleotide bases in length, from 3,700 to 3,800 nucleotide bases in length, from 3,800 to 3,900 nucleotide bases in length, or from 3,900 to 4,000 nucleotide bases in length, e.g., about 3,108 nucleotide bases in length, about 3,285 nucleotide bases in length, about 3,375 nucleotide bases in length, about 3,503 nucleotide bases in length, about 3,630 nucleotide bases in length, about 3,540 nucleotide bases in length, about 3,363 nucleotide bases in length, about 3,273 nucleotide bases in length, about 3,145 nucleotide bases in length, or about 3,018 nucleotide bases in length).

### Binding Domains

Trans-splicing molecules of the invention feature a binding domain configured to bind a target CEP290 intron. In one embodiment, the binding domain is a nucleic acid sequence complementary to a sequence of the target CEP290 pre-mRNA (e.g., a target CEP290 intron) to suppress endogenous target cis-splicing while enhancing trans-splicing between the trans-spicing molecule and the target CEP290 pre-mRNA, e.g., to create a chimeric molecule having a portion of endogenous CEP290 mRNA and the coding domain having one or more functional CEP290 exons. In some embodiments, the binding domain is in an antisense orientation to a sequence of the target CEP290 intron.

A 5′ trans-splicing molecule will generally bind the target CEP290 intron 3′ to the mutation. In one embodiment, the binding domain comprises a part of a sequence complementary to the target CEP290 intron.

In another embodiment, the binding domain is targeted to an intron sequence in close proximity to the 3′ or 5′ splice signals of a target intron. In still another embodiment, a binding domain sequence can bind to the target intron in addition to part of an adjacent exon.

Thus, in some instances, the binding domain binds specifically to the mutated endogenous target pre-mRNA to anchor the coding domain of the trans-splicing molecule to the pre-mRNA to permit trans-splicing to occur at the correct position in the target CEP290 gene. The spliceosome processing machinery of the nucleus may then mediate successful trans-splicing of the corrected exon for the mutated exon causing the disease.

In certain embodiments, the trans-splicing molecules feature binding domains that contain sequences on the target pre-mRNA that bind in more than one place. The binding domain may contain any number of nucleotides necessary to stably bind to the target pre-mRNA to permit trans-splicing to occur with the coding domain. In one embodiment, the binding domains are selected using mFOLD structural analysis for accessible loops (Zuker, Nucleic Acids Res. 2003, 31 (13): 3406-3415).

Suitable target binding domains can be from 10 to 500 nucleotides in length. In some embodiments, the binding domain is from 20 to 400 nucleotides in length. In some embodiments, the binding domain is from 50 to 300 nucleotides in length. In some embodiments, the binding domain is from 100 to 200 nucleotides in length. In some embodiments, the binding domain is from 10-20 nucleotides in length (e.g., 10, 11, 12, 13, 14, 15, 16, 17, 18, 19, or 20 nucleotides in length), 20-30 nucleotides in length (e.g., 20, 21, 22, 23, 24, 25, 26, 27, 28, 29, or 30 nucleotides in length), 30-40 nucleotides in length (e.g., 30, 31, 32, 33, 34, 35, 36, 37, 38, 39, or 40 nucleotides in length), 40-50 nucleotides in length (e.g., 40, 41, 42, 43, 44, 45, 46, 47, 48, 49, 50 nucleotides in length), 50-60 nucleotides in length (e.g., 50, 51, 52, 53, 54, 55, 56, 57, 58, 59, or 60 nucleotides in length), 60-70 nucleotides in length (e.g., 60, 61, 62, 63, 64, 65, 66, 67, 68, 69, or 70 nucleotides in length), 70-80 nucleotides in length (e.g., 70, 71, 72, 73, 74, 75, 76, 77, 78, 79, or 80 nucleotides in length), 80-90 nucleotides in length (e.g., 80, 81, 82, 83, 84, 85, 86, 87, 88, 89, or 90 nucleotides in length), 90-100 nucleotides in length (e.g., 90, 91, 92, 93, 94, 95, 96, 97, 98, 99, or 100 nucleotides in length), 100-110 nucleotides in length (e.g., 100, 101, 102, 103, 104, 105, 106, 107, 108, 109, or 110 nucleotides in length), 110-120 nucleotides in length (e.g., 110, 111, 112, 113, 114, 115, 116, 117, 118, 119, or 120 nucleotides in length), 120-130 nucleotides in length (e.g., 120, 121, 122, 123, 124, 125, 126, 127, 128, 129, or 130 nucleotides in length), 130-140 nucleotides in length (e.g., 130, 131, 132, 133, 134, 135, 136, 137, 138, 139, or 140 nucleotides in length), 140-150 nucleotides in length (e.g., 140, 141, 142, 143, 144, 145, 146, 147, 148, 149, or 150 nucleotides in length), 150-160 nucleotides in length (e.g., 150, 151, 152, 153, 154, 155, 156, 157, 158, 159, or 160 nucleotides in length), 160-170 nucleotides in length (e.g., 160, 161, 162, 163, 164, 165, 166, 167, 168, 169, or 170 nucleotides in length), 170-180 nucleotides in length (e.g., 170, 171, 172, 173, 174, 175, 176, 177, 178, 179, or 180 nucleotides in length), 180-190 nucleotides in length (e.g., 180, 181, 182, 183, 184, 185, 186, 187, 188, 189, or 190 nucleotides in length), 190-200 nucleotides in length (e.g., 190, 191, 192, 193, 194, 195, 196, 197, 198, 199, or 200 nucleotides in length), 200-210 nucleotides in length, 210-220 nucleotides in length, 220-230 nucleotides in length, 230-240 nucleotides in length, 240-250 nucleotides in length, 250-260 nucleotides in length, 260-270 nucleotides in length, 270-280 nucleotides in length, 280-290 nucleotides in length, 290-300 nucleotides in length, 300-350 nucleotides in length, 350-400 nucleotides in length, 400-450 nucleotides in length, or 450-500 nucleotides in length. In some embodiments, the binding domain is about 150 nucleotides in length. In another embodiment, the target binding domains may include a nucleic acid sequence up to 750 nucleotides in length. In another embodiment, the target binding domains may include a nucleic acid sequence up to 1000 nucleotides in length. In another embodiment, the target binding domains may include a nucleic acid sequence up to 2000 nucleotides or more in length.

In some embodiments, the specificity of the trans-splicing molecule may be increased by increasing the length of the target binding domain. Other lengths may be used depending upon the lengths of the other components of the trans-splicing molecule.

The binding domain may be from 80% to 100% complementary to the target intron to be able to hybridize stably with the target intron. For example, in some embodiments, the binding domain is 80%, 81%, 82%, 83%, 84%, 85%, 86%, 87%, 88%, 89%, 90%, 91%, 92%, 93%, 94%, 95%, 96%, 97%, 98%, 99%, or 100% complimentary to the target intron. The degree of complementarity is selected by one of skill in the art based on the need to keep the trans-splicing molecule and the nucleic acid construct containing the necessary sequences for expression and for inclusion in the rAAV within a 3,000 or up to 4,000 nucleotide base limit. The selection of this sequence and strength of hybridization depends on the complementarity and the length of the nucleic acid.

Any of the aforementioned binding domains may bind to a binding site within intron 26 (SEQ ID NO: 85; e.g., at or 3′ to a mutation, e.g., a substitution mutation at nucleotide 1,655 of intron 26), intron 27 (SEQ ID NO: 86), intron 28 (SEQ ID NO: 87), intron 29 (SEQ ID NO: 88), or intron 30 (SEQ ID NO: 89).

In certain instances of the invention, the trans-splicing molecule features a binding domain that binds to intron 26 of CEP290 (SEQ ID NO: 85) and includes a coding domain having functional CEP290 exons 2-26. In some embodiments, the binding site comprises any one or more of nucleotides 4,980 to 5,383 of SEQ ID NO: 85. In one embodiment, the binding site comprises any one or more of nucleotides 5,348 to 5,838 of SEQ ID NO: 85 (e.g., any one or more of nucleotides 5,348 to 5,700 of SEQ ID NO: 85, e.g., any one or more of nucleotides 5,400 to 5,600 of SEQ ID NO: 85, e.g., any one or more of nucleotides 5,460 to 5,560 of SEQ ID NO: 85, e.g., at least nucleotide 5,500 of SEQ ID NO: 85).

In other embodiments, the trans-splicing molecule features a binding domain that binds to intron 27 of CEP290 (SEQ ID NO: 86) and includes a coding domain having functional CEP290 exons 2-27. In some embodiments, the binding site comprises any one or more of nucleotides 120 to 680, nucleotides 710 to 2,200, or nucleotides 2,670 to 2,910 of SEQ ID NO: 86. In some embodiments, the binding site comprises any one or more of nucleotides 790 to 2,100 of SEQ ID NO: 86, e.g., any one or more of nucleotides 1,020 to 1,630 of SEQ ID NO: 86. In other embodiments, the binding site comprises any one or more of nucleotides 1,670 to 2,000 of SEQ ID NO: 86.

In some embodiments, the trans-splicing molecule features a binding domain that binds to intron 28 of CEP290 (SEQ ID NO: 87) and includes a coding domain having functional CEP290 exons 2-28. In some embodiments, the binding site comprises any one or more of nucleotides 1 to 390, nucleotides 410 to 560, or nucleotides 730 to 937 of SEQ ID NO: 87. In some embodiments, the binding site comprises any one or more of nucleotides 1 to 200 of SEQ ID NO: 87. In other embodiments, the binding site comprises any one or more of nucleotides 720 to 900 of SEQ ID NO: 87.

In some embodiments, the trans-splicing molecule features a binding domain that binds to intron 29 of CEP290 (SEQ ID NO: 88) and includes a coding domain having functional CEP290 exons 2-29. In some embodiments, the binding site comprises any one or more of nucleotides 1 to 600, nucleotides 720 to 940, or nucleotides 1,370 to 1,790 of SEQ ID NO: 88.

In other embodiments, the trans-splicing molecule features a binding domain that binds to intron 30 of CEP290 (SEQ ID NO: 89) and includes a coding domain having functional CEP290 exons 2-30. In some embodiments, the binding site comprises any one or more of nucleotides 880 to 1,240 of SEQ ID NO: 89, e.g., any one or more of nucleotides 950 to 1,240 of SEQ ID NO: 89, e.g., any one or more of nucleotides 1,060 to 1,240 of SEQ ID NO: 89.

### Splicing Domains

The following splicing domains can be used in any of the trans-splicing molecules of the invention (e.g., any of the ABCA4 trans-splicing molecules or CEP290 trans-splicing molecules described herein).

The splicing domain can include a splice site, a branch point, and/or a PPT tract to mediate trans-splicing. In some embodiments, a splicing domain has a single splice site, which denotes that the splice site enables trans-splicing, but not cis-splicing, due to the lack of a corresponding splice site. In some embodiments, the splicing domains of the 3′ trans-splicing molecule include a strong conserved branch point or branch site sequence, a polypyrimidine tract (PPT), and a 3′ splice acceptor (AG or YAG) site and/or a 5′ splice donor site. The splicing domains of the 5′ trans-splicing molecule do not contain the branch point or PPT, but comprise a 5′ splice acceptor/or 3′ splice donor splice site.

Splicing domains may be selected by one of skill in the art according to known methods and principles. The splicing domain provides essential consensus motifs that are recognized by the spliceosome. The use of branch point and PPT follows consensus sequences required for performance of the two phosphoryl transfer reaction involved in trans-splicing. In one embodiment a branch point consensus sequence in mammals is YNYURAC (Y=pyrimidine; N=any nucleotide). A polypyrimidine tract is located between the branch point and the splice site acceptor and is important for different branch point utilization and 3′ splice site recognition. Consensus sequences for the 5′ splice donor site and the 3′ splice region used in RNA splicing are well known in the art. In addition, modified consensus sequences that maintain the ability to function as 5′ donor splice sites and 3′ splice regions may be used. Briefly, in one embodiment, the 5′ splice site consensus sequence is the nucleic acid sequence AG/GURAGU (where / indicates the splice site). In another embodiment the endogenous splice sites that correspond to the exon proximal to the splice site can be employed to maintain any splicing regulatory signals.

In one embodiment, a suitable 5′ splice site with spacer is: 5′-GTA AGA GAG CTC GTT GCG ATA TTA T-3′ (SEQ ID NO: 1). In one embodiment, a suitable 5′ splice site is AGGT.

In one embodiment, a suitable 3′ trans-splicing molecule branch site is 5′-TACTAAC-3′. In one embodiment, a suitable 3′ splice site is: 5′-TAC TAA CTG GTA CCT CTT CTT TTT TTT CTG CAG-3′ (SEQ ID NO: 2) or 5′-CAGGT-3′. In one embodiment, a suitable 3′ trans-splicing molecule PPT is: 5′-TGG TAC CTC TTC TTT TTT TTC TG-3′ (SEQ ID NO: 3).

### Additional Components or Modifications

In some embodiments of any of the trans-splicing molecules of the invention (e.g., any of the ABCA4 trans-splicing molecules or CEP290 trans-splicing molecules described herein), the splicing domain is included as part of an artificial intron, which may include one or more additional components. For example, a spacer region may be included within an artificial intron to separate the splicing domain from the target binding domain in the trans-splicing molecule. The spacer region may be designed to include features such as (i) stop codons which would function to block translation of any unspliced trans-splicing molecule and/or (ii) sequences that enhance trans-splicing to the target pre-mRNA. The spacer may be between 3 to 25 nucleotides or more depending upon the lengths of the other components of the trans-splicing molecule and the rAAV limitations. In one embodiment, a suitable 5′ trans-splicing molecule spacer is AGA TCT CGT TGC GAT ATT AT (SEQ ID NO: 4). In one embodiment, a suitable 3′ spacer is: 5′-GAG AAC ATT ATT ATA GCG TTG CTC GAG-3′ (SEQ ID NO: 5).

Still other optional components of the trans-splicing molecules (e.g., as part of artificial introns) include mini introns, and intronic or exonic enhancers (e.g., intronic splice enhancers, e.g., downstream intronic splice enhancers) or silencers that would regulate the trans-splicing.

In another embodiment, the trans-splicing molecule further comprises (e.g., as part of an artificial intron) at least one safety sequence incorporated into the spacer, binding domain, or elsewhere in the trans-splicing molecule to prevent nonspecific trans-splicing. This is a region of the trans-splicing molecule that covers elements of the 3′ and/or 5′ splice site of the trans-splicing molecule by relatively weak complementarity, preventing non-specific trans-splicing. The trans-splicing molecule is designed in such a way that upon hybridization of the binding/targeting portion(s) of the trans-splicing molecule, the 3′ or 5′ splice site is uncovered and becomes fully active. Such safety sequences comprise a complementary stretch of cis-sequence (or could be a second, separate, strand of nucleic acid) which binds to one or both sides of the trans-splicing molecule branch point, pyrimidine tract, 3′ splice site and/or 5′ splice site (splicing elements), or could bind to parts of the splicing elements themselves. The binding of the safety sequence may be disrupted by the binding of the target binding region of the trans-splicing molecule to the target pre-mRNA, thus exposing and activating the splicing elements (making them available to trans-splice into the target pre-mRNA). In another embodiment, the trans-splicing molecule has 3′ UTR sequences or ribozyme sequences added to the 3′ or 5′ end.

In an embodiment, splicing enhancers such as, for example, sequences referred to as exonic splicing enhancers may also be included in the structure of an artificial intron. Additional features can be added to the artificial intron, such as polyadenylation signals to modify RNA expression/stability, or 5′ splice sequences to enhance splicing, additional binding regions, safety-self complementary regions, additional splice sites, or protective groups to modulate the stability of the molecule and prevent degradation. In addition, stop codons may be included in the trans-splicing molecule (e.g., as part of the artificial intron) structure to prevent translation of unspliced trans-splicing molecules. Additional elements, such as a 3′ hairpin structure, circularized RNA, nucleotide base modification, or synthetic analogs can be incorporated into trans-splicing molecules to promote or facilitate nuclear localization and spliceosomal incorporation, and intra-cellular stability.

In some embodiments, binding of a trans-splicing molecule to the target pro-mRNA is mediated by complementarity (i.e. based on base-pairing characteristics of nucleic acids), triple helix formation, or protein-nucleic acid interaction (as described in documents cited herein). In one embodiment, the nucleic acid trans-splicing molecule includes DNA, RNA, or DNA/RNA hybrid molecules, wherein the DNA or RNA is either single or double stranded. Also included herein are RNAs or DNAs, which can hybridize to one of the aforementioned RNAs or DNAs, preferably under stringent conditions, for example, at 60° C. in 2.5× SSC buffer and several washes at 37° C. at a lower buffer concentration, for example, 0.5× SSC buffer. These nucleic acids can encode proteins exhibiting lipid phosphate phosphatase activity and/or association with plasma membranes. When trans-splicing molecules are synthesized in vitro, such trans-splicing molecules can be modified at the base moiety, sugar moiety, or phosphate backbone, for example, to improve stability of the molecule, hybridization to the target mRNA, transport into the cell, stability in the cells to enzymatic cleavage, etc. For example, modification of a trans-splicing molecule to reduce the overall charge can enhance the cellular uptake of the molecule. In addition modifications can be made to reduce susceptibility to nuclease or chemical degradation. The nucleic acid molecules may be synthesized in such a way as to be conjugated to another molecule, e.g., a peptide, hybridization triggered cross-linking agent, transport agent, hybridization-triggered cleavage agent, etc.

Various other well-known modifications to the nucleic acid molecules can be introduced as a means of increasing intracellular stability and half-life (see also above for oligonucleotides). Possible modifications are known to the art. Modifications, which may be made to the structure of synthetic trans-splicing molecules include backbone modifications.

### III. Recombinant AAV Molecules

Any suitable nucleic acid vector may be used in conjunction with the present compositions and methods to design and assemble the components of the trans-splicing molecule and a recombinant adeno-associated virus (AAV). In one embodiment, the vector is a recombinant AAV carrying the trans-splicing molecule and driven by a promoter that expresses a trans-splicing molecule in selected cells of a subject. Methods for assembly of the recombinant vectors are known in the art. See, e.g., Ausubel et al., Current Protocols in Molecular Biology, John Wiley & Sons, New York, 1989; Kay, M. A. et al., Nat. Medic, 2001, 7 (I): 33-40; and Walther W. and Stein U., Drugs 2000, 60 (2): 249-71.

In certain embodiments described herein, the trans-splicing molecule carrying the ABCA4 gene binding and coding domains is delivered to the selected cells, e.g., photoreceptor cells, in need of treatment by means of an AAV vector. More than 30 naturally occurring serotypes of AAV are available. Many natural variants in the AAV capsid exist, allowing identification and use of an AAV with properties specifically suited for ocular cells. AAV viruses may be engineered by conventional molecular biology techniques, making it possible to optimize these particles for cell specific delivery of the trans-splicing molecule nucleic acid sequences, for minimizing immunogenicity, for tuning stability and particle lifetime, for efficient degradation, for accurate delivery to the nucleus, etc.

The expression of the trans-splicing molecules described herein can be achieved in the selected cells through delivery by recombinantly engineered AAVs or artificial AAVs that contain sequences encoding the desired trans-splicing molecule. The use of AAVs is a common mode of exogenous delivery of DNA as it is relatively non-toxic, provides efficient gene transfer, and can be easily optimized for specific purposes. Among the well-characterized serotypes of AAVs isolated from human or non-human primates, human serotype 2 has been widely used for efficient gene transfer experiments in different target tissues and animal models. Other AAV serotypes include, but are not limited to, AAV1, AAV3, AAV4, AAV5, AAV6, AAV7, AAV8, and AAV9. Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV8, AAV9 or other known and unknown AAV serotypes. In one embodiment, the ITRs are from AAV2. These ITRs or other AAV components may be readily isolated using techniques available to those of skill in the art from an AAV serotype. Such AAV may be isolated or obtained from academic, commercial, or public sources (e.g., the American Type Culture Collection, Manassas, VA). Alternatively, the AAV sequences may be obtained through synthetic or other suitable means by reference to published sequences such as are available in the literature or in databases such as, e.g., GenBank, PubMed, or the like.

Desirable AAV fragments for assembly into vectors include the cap proteins, including the vp1, vp2, vp3, and hypervariable regions, the rep proteins, including rep 78, rep 68, rep 52, and rep 40, and the sequences encoding these proteins. These fragments may be readily utilized in a variety of vector systems and host cells. Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a pseudotyped AAV, a chimeric AAV capsid, a recombinant AAV capsid, or a “humanized” AAV capsid. Pseudotyped vectors, wherein the capsid of one AAV is utilized with the ITRs from an AAV having a different capsid protein, are useful in the invention. In one embodiment, the AAV is AAV2/5 (i.e., an AAV having AAV2 ITRs and an AAV5 capsid). In another embodiment, the AAV is AAV2/8 (i.e., an AAV having AAV2 ITRs and an AAV8 capsid). In one embodiment, the AAV includes an AAV8 capsid. Such AAV8 capsid includes the amino acid sequence found under NCBI Reference Sequence: YP_077180.1 (SEQ ID NO: 56). In another embodiment, the AAV8 capsid includes a capsid encoded by nt 2121 to 4337 of GenBank accession: AF513852.1 (SEQ ID NO: 57).

In one embodiment, the AAV includes a capsid sequence derived from AAV8. In some embodiments, the AAV derived from AAV8 is AAV8(b), described in U.S. Pat. No. 9,567,376, which is incorporated herein by reference in its entirety. AAV(b) (SEQ ID NO: 58) comprises the amino acid sequence of Pro-Glu-Arg-Thr-Ala-Met-Ser-Leu-Pro at amino acid positions 587-595 as compared to wildtype AAV8. In another embodiment, the AAV8(b) capsid is encoded by SEQ ID NO: 59.

In one embodiment, the vectors useful in compositions and methods described herein contain, at a minimum, sequences encoding a selected AAV serotype capsid, e.g., an AAV2 capsid, or a fragment thereof. In another embodiment, useful vectors contain, at a minimum, sequences encoding a selected AAV serotype rep protein, e.g., AAV2 rep protein, or a fragment thereof. Optionally, such vectors may contain both AAV cap and rep proteins. In vectors in which both AAV rep and cap are provided, the AAV rep and AAV cap sequences can both be of one serotype origin, e.g., an AAV2 origin.

Alternatively, vectors may be used in which the rep sequences are from an AAV serotype which differs from that which is providing the cap sequences. In one embodiment, the rep and cap sequences are expressed from separate sources (e.g., separate vectors, or a host cell and a vector). In another embodiment, these rep sequences are fused in frame to cap sequences of a different AAV serotype to form a chimeric AAV vector described in U.S. Pat. No. 7,282,199, which is incorporated by reference herein.

A suitable recombinant AAV (rAAV) is generated by culturing a host cell which contains a nucleic acid sequence encoding an AAV serotype capsid protein, or fragment thereof, as defined herein; a functional rep gene; a minigene composed of, e.g., AAV ITRs and the trans-splicing molecule nucleic acid sequence; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

In one embodiment, the AAV includes a promoter (or a functional fragment of a promoter). The selection of the promoter to be employed in the rAAV may be made from among a wide number of constitutive or inducible promoters that can express the selected transgene in the desired target cell. See, e.g., the list of promoters identified in International Patent Publication No. WO 2014/012482, incorporated by reference herein. In one embodiment, the promoter is cell-specific. The term “cell-specific” means that the particular promoter selected for the recombinant vector can direct expression of the selected transgene in a particular cell type. In one embodiment, the promoter is specific for expression of the transgene in photoreceptor cells. In another embodiment, the promoter is specific for expression in the rods and/or cones. In another embodiment, the promoter is specific for expression of the transgene in retinal pigment epithelium (RPE) cells. In another embodiment, the promoter is specific for expression of the transgene in ganglion cells. In another embodiment, the promoter is specific for expression of the transgene in Mueller cells. In another embodiment, the promoter is specific for expression of the transgene in bipolar cells. In another embodiment, the promoter is specific for expression of the transgene in horizontal cells. In another embodiment, the promoter is specific for expression of the transgene in amacrine cells. In another embodiment, the transgene is expressed in any of the above noted cells.

In another embodiment, the promoter is the native promoter for the target gone to be expressed. Useful promoters include, without limitation, a rod opsin promoter, a red-green opsin promoter, a blue opsin promoter, a cGMP-phosphodiesterase promoter, a mouse opsin promoter, a rhodopsin promoter, an alpha-subunit of cone transducing, a beta phosphodiesterase (PDE) promoter, a retinitis pigmentosa promoter, a NXNL2/NXNL 1 promoter, the RPE65 promoter, the retinal degeneration slow/peripherin 2 (Rds/perph2) promoter, and the VMD2 promoter.

Other conventional regulatory sequences contained in the mini-gene or rAAV are also disclosed in documents such as WO 2014/124282 and others cited and incorporated by reference herein. One of skill in the art may make a selection among these, and other, expression control sequences without departing from the scope described herein

An AAV minigene may include the trans-splicing molecule described herein and its regulatory sequences, and 5′ and 3′ AAV ITRs. In one embodiment, the ITRs of AAV serotype 2 are used. In another embodiment, the ITRs of AAV serotype 5 or 8 are used. However, ITRs from other suitable serotypes may be selected. In some embodiments, the minigene is packaged into a capsid protein and delivered to a selected host cell.

The minigene, rep sequences, cap sequences, and helper functions required for producing the rAAV may be delivered to the packaging host cell in the form of any genetic element which transfers the sequences carried thereon. The selected genetic element may be delivered by any suitable method, including those described herein. The methods used to construct any embodiment described herein are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, NY. Similarly, methods of generating rAAV virions are well known and the selection of a suitable method is not a limitation on the present invention. See, e.g., K. Fisher et al., J. Virol., 1993 70:520-532 and U.S. Pat. No. 5,478,745, each of which is incorporated by reference herein.

In another embodiment, the trans-splicing molecule minigene is prepared in a proviral plasmid, such as those disclosed in International Patent Publication No. WO 2012/158757, incorporated herein by reference. Such a proviral plasmid contains a modular recombinant AAV genome comprising in operative association comprising: a wildtype 5′ AAV2 ITR sequence flanked by unique restriction sites that permit ready removal or replacement of said ITR; a promoter comprising a 49-nucleic acid cytomegalovirus sequence upstream of a cytomegalovirus (CMV)-chicken beta actin sequence, or a photoreceptor-specific promotor/enhancer, the promoter flanked by unique restriction sites that permit ready removal or replacement of the entire promoter sequence, and the upstream sequence flanked by unique restriction sites that permit ready removal or replacement of only the upstream CMV or enhancer sequence, from the promoter sequence. The trans-splicing molecule described herein can be inserted into the site of a multi-cloning poly linker, wherein the trans-splicing molecule is operatively linked to, and under the regulatory control of, the promoter. A bovine growth hormone polyadenylation sequence flanked by unique restriction sites that permit ready removal or replacement of said polyA sequence; and a wildtype 3′ AAV2 ITR sequence flanked by unique restriction sites that permit ready removal or replacement of the 3′ ITR; are also part of this plasmid. The plasmid backbone comprises the elements necessary for replication in bacterial cells, e.g., a kanamycin resistance gene, and is itself flanked by transcriptional terminator/insulator sequences.

In one embodiment, a proviral plasmid comprises: (a) a modular recombinant AAV genome comprising in operative association comprising: (i) a wildtype 5′ AAV2 ITR sequence flanked by unique restriction sites that permit ready removal or replacement of said ITR; (ii) a promoter comprising (A) a 49-nucleic acid CMV sequence upstream of a CMV-chicken beta actin sequence; (b) a photoreceptor-specific promoter/enhancer; or (c) a neuronal cell-specific promoter/enhancer. The promoter is flanked by unique restriction sites that permit ready removal or replacement of the entire promoter sequence, and the upstream sequence flanked by unique restriction sites that permit ready removal or replacement of only the upstream CMV or enhancer sequence, from the promoter sequence. Also part of this proviral plasmid is a multi-cloning polylinker sequence that permits insertion of a trans-splicing molecule sequence including any of those described herein, wherein the trans-splicing molecule is operatively linked to, and under the regulatory control of, the promoter; a bovine growth hormone polyadenylation sequence flanked by unique restriction sites that permit ready removal or replacement of said polyA sequence; and a wildtype 3′ AAV2 ITR sequence flanked by unique restriction sites that permit ready removal or replacement of the 3′ ITR. The proviral plasmid also contains a plasmid backbone comprising the elements necessary for replication in bacterial cells, and further comprising a kanamycin resistance gene, said plasmid backbone flanked by transcriptional terminator/insulator sequences. The proviral plasmid described herein may also contain in the plasmid backbone a non-coding lambda phage 5.1 kb stuffer sequence to increase backbone length and prevent reverse packaging of non-functional AAV genomes.

In some embodiments, a proviral plasmid contains multiple copies of a trans-splicing molecule. For example, the present invention features trans-splicing molecules that are less than half the packaging limit for AAV and can therefore be repeated once, twice, three times, four times, five times, six times, seven times, eight times, nine times, 10 times, 11 times, 12 times, 13 times, 14 times, 15 times, 16 times, 17 times, 18 times, 19 times, 20 times, or more on a single proviral plasmid.

In yet a further aspect, the promoter of the proviral plasmid is modified to reduce the size of the promoter to permit larger trans-splicing molecule sequences to be inserted in the rAAV. In one embodiment, the CMV/CBA hybrid promoter, which normally includes a non-coding exon and intron totaling about 1,000 base pairs, is replaced with a 130-base pair chimeric intron, as described in International Patent Publication No. WO 2017/087900, which is incorporated herein by reference in its entirety.

These proviral plasmids are then employed in currently conventional packaging methodologies to generate a recombinant virus expressing the trans-splicing molecule transgene carried by the proviral plasmids. Suitable production cell lines are readily selected by one of skill in the art. For example, a suitable host cell can be selected from any biological organism, including prokaryotic (e.g., bacterial) cells, and eukaryotic cells, including, insect cells, yeast cells and mammalian cells. Briefly, the proviral plasmid is transfected into a selected packaging cell, where it may exist transiently. Alternatively, the minigene or gene expression cassette with its flanking ITRs is stably integrated into the genome of the host cell, either chromosomally or as an episome. Suitable transfection techniques are known and may readily be utilized to deliver the recombinant AAV genome to the host cell. Typically, the proviral plasmids are cultured in the host cells which express the cap and/or rep proteins. In the host cells, the minigene consisting of the trans-splicing molecule with flanking AAV ITRs is rescued and packaged into the capsid protein or envelope protein to form an infectious viral particle. Thus, a recombinant AAV infectious particle is produced by culturing a packaging cell carrying the proviral plasmid in the presence of sufficient viral sequences to permit packaging of the gene expression cassette viral genome into an infectious AAV envelope or capsid.

### IV. Pharmaceutical Compositions and Kits

Provided herein are pharmaceutical compositions including a nucleic acid trans-splicing molecule, a proviral plasmid, or a rAAV comprising the nucleic acid trans-splicing molecule described herein. In some embodiments, the pharmaceutical composition includes any of the 5′ trans-splicing molecules described herein. In other embodiments, the pharmaceutical composition includes any of the 3′ trans-splicing molecules described herein. In some embodiments, the pharmaceutical composition includes a 5′ trans-splicing molecule and a 3′ trans-splicing molecule, e.g., wherein the 5′ trans-splicing molecule and the 3′ trans-splicing molecule together contain functional ABCA4 exons 1-50 and bind the same target ABCA4 intron.

The pharmaceutical compositions described herein may be assessed for contamination by conventional methods and then formulated into a pharmaceutical composition intended for a suitable route of administration. Still other compositions containing the trans-splicing molecule, e.g., naked DNA or as protein, may be formulated similarly with a suitable carrier. Such formulation involves the use of a pharmaceutically and/or physiologically acceptable vehicle or carrier, particularly directed for administration to the target cell. In one embodiment, carriers suitable for administration to the target cells include buffered saline, an isotonic sodium chloride solution, or other buffers, e.g., HEPES, to maintain pH at appropriate physiological levels, and, optionally, other medicinal agents, pharmaceutical agents, stabilizing agents, buffers, carriers, adjuvants, diluents, etc.

In some embodiments, the carrier is a liquid for injection. Exemplary physiologically acceptable carriers include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween20.

In other embodiments, compositions containing trans-splicing molecules described herein include a surfactant. Useful surfactants, such as Pluronic F68 (Poloxamer 188, also known as LUTROL® F68) may be included as they prevent AAV from sticking to inert surfaces and thus ensure delivery of the desired dose. As an example, one illustrative composition designed for the treatment of the ocular diseases described herein comprises a recombinant adeno-associated vector carrying a nucleic acid sequence encoding 3′ trans-splicing molecule as described herein, under the control of regulatory sequences which express the trans-splicing molecule in an ocular cell of a mammalian subject, and a pharmaceutically acceptable carrier. The carrier is isotonic sodium chloride solution and includes a surfactant Pluronic F68. In one embodiment, the trans-splicing molecule is any of those described herein.

In yet another exemplary embodiment, the composition comprises a recombinant AAV2/5 pseudotyped adeno-associated virus carrying a 3′ or 5′ trans-splicing molecule for ABCA4 gene replacement, the nucleic acid sequence under the control of promoter which directs expression of the trans-splicing molecule in said photoreceptor cells, wherein the composition is formulated with a carrier and additional components suitable for subretinal injection. In still another embodiment, the composition or components for production or assembly of this composition, including carriers, rAAV particles, surfactants, and/or the components for generating the rAAV, as well as suitable laboratory hardware to prepare the composition, may be incorporated into a kit.

In some instances, the composition comprises a recombinant AAV2/5 pseudotyped adeno-associated virus carrying a 5′ trans-splicing molecule for CEP290 gene replacement, the nucleic acid sequence under the control of promoter which directs expression of the trans-splicing molecule in said photoreceptor cells, wherein the composition is formulated with a carrier and additional components suitable for subretinal injection. In still another embodiment, the composition or components for production or assembly of this composition, including carriers, rAAV particles, surfactants, and/or the components for generating the rAAV, as well as suitable laboratory hardware to prepare the composition, may be incorporated into a kit.

Additionally provided herein are kits containing a first pharmaceutical composition comprising a 5′ trans-splicing molecule and a second pharmaceutical composition comprising a 3′ trans-splicing molecule, e.g., wherein the 5′ trans-splicing molecule and the 3′ trans-splicing molecule together contain functional ABCA4 exons 1-50 and bind the same target ABCA4 intron (e.g., wherein the trans-splicing molecules are packaged in any AAV vectors described herein). In some embodiments, the kit includes instructions for mixing the two pharmaceutical compositions prior to administration.

Additionally provided herein are kits containing a first pharmaceutical composition comprising a 5′ trans-splicing molecule to bind a target CEP290 intron.

### V. Methods

The compositions described above involving ABCA4 trans-splicing are useful in methods of treating diseases or disorders caused by a mutation in the ABCA4 gene, such as a Stargardt Disease (e.g., Stargardt Disease 1) including delaying or ameliorating symptoms associated with the disease described herein. Such methods involve contacting a target ABCA4 gene (e.g., ABCA4 pre-mRNA) with a trans-splicing molecule as described herein (e.g., one or more of a 3′trans-splicing molecule, 5′ trans-splicing molecule, or both 3′ and 5′ trans-splicing molecule as described herein), under conditions in which a coding domain of the trans-splicing molecule is spliced to the target ABCA4 gene to replace a part of the targeted gene carrying one or more defects or mutations, with a functional (i.e., healthy), or normal or wildtype or corrected mRNA of the targeted gene, in order to correct expression of ABCA4 in the target cell. Thus, the methods and compositions are used to treat the ocular diseases/pathologies associated with the specific mutations and/or gene expression.

In one embodiment, the contacting involves direct administration to the affected subject. In another embodiment, the contacting may occur ex vivo to the cultured cell and the treated ocular cell reimplanted in the subject. In one embodiment, the method involves administering a rAAV carrying a 3′ trans-splicing molecule. In another embodiment, the method involves administering a rAAV carrying a 5′ trans-splicing molecule. In still another embodiment, the method involves administering a mixture of rAAV carrying a 3′ trans-splicing molecule and rAAV carrying a 5′ trans-splicing molecule. These methods comprise administering to a subject in need thereof an effective concentration of a composition of any of those described herein.

In some embodiments, the methods include selecting one or more trans-splicing molecules for treating a subject having a disorder associated with a mutation in ABCA4, such as Stargardt Disease (e.g., Stargardt Disease 1). Such selection can be based on the genotype of the subject. In some embodiments, a disorder associated with ABCA4 may be an autosomal recessive disorder. In some instances, the subject is homozygous or compound heterozygous for the mutation in ABCA4. Methods of screening for and identifying particular mutations in ABCA4 are known in the art.

In other instances, the compositions described above involving CEP290 trans-splicing are useful in methods of treating diseases or disorders caused by a mutation in the CEP290 gene, such as Leber congenital amourosis (e.g., LCA 10) including delaying or ameliorating symptoms associated with the disease described herein. Such methods involve contacting a target CEP290 gene (e.g., CEP290 pre-mRNA) with a trans-splicing molecule as described herein (e.g., a 5′ trans-splicing molecule), under conditions in which a coding domain of the trans-splicing molecule is spliced to the target CEP290 gene to replace a part of the targeted gene carrying one or more defects or mutations, with a functional (i.e., healthy), or normal or wildtype or corrected mRNA of the targeted gene, in order to correct expression of CEP290 in the target cell. Thus, the methods and compositions are used to treat the ocular diseases/pathologies associated with the specific mutations and/or gene expression. Methods of the invention include correcting a pathogenic point mutation in intron 26 (e.g., at nucleotide 1,655 of intron 26) of CEP290 by administering a 5′ trans-splicing molecule (e.g., any of the 5′ trans-splicing molecules described herein), or a pharmaceutical composition thereof. Thus, the invention provides methods of treating a subject having a disease or disorder associated with a mutation in CEP290 (e.g., a disease or disorder associated with a mutation in intron 26 of CEP290, e.g., at nucleotide 1,655 of intron 26) by administering a trans-splicing molecule described herein. Any of the aforementioned trans-splicing molecules can be included in a pharmaceutical composition (e.g., a single pharmaceutical composition including both molecules, either pre-prepared or admixed prior to administration, e.g., as part of a kit).

In one embodiment, the contacting involves direct administration to the affected subject. In another embodiment, the contacting may occur ex vivo to the cultured cell and the treated ocular cell reimplanted in the subject. In one embodiment, the method involves administering a rAAV carrying a 5′ trans-splicing molecule. These methods comprise administering to a subject in need thereof an effective concentration of a composition of any of those described herein.

In some embodiments, the methods include selecting one or more trans-splicing molecules for treating a subject having a disorder associated with a mutation in CEP290, such as LCA 10. Such selection can be based on the genotype of the subject. In some embodiments, a disorder associated with CEP290 may be an autosomal recessive disorder. In some instances, the subject is homozygous or compound heterozygous for the mutation in CEP290. Methods of screening for and identifying particular mutations in CEP290 are known in the art.

### Single Trans-Splicing Molecules for Correcting a Single Mutation

Methods of the invention include selecting a single trans-splicing molecule based on the location of a single mutation in ABCA4 (e.g., a mutation of one allele of the subject). In some instances in the context of autosomal recessive mutations, correction of just one of two mutations can be sufficient to restore functional protein activity, for example, wherein the second allele has a mutation on the opposite portion of the ABCA4 gene, out of range of a single AAV-delivered trans-splicing molecule configured to correct the first mutation.

Thus, in some embodiments, methods of the invention include selecting a single trans-splicing molecule to correct a single mutation on the 5′ portion of the target gene, e.g., without regard to the location of the mutation in the other allele. In one embodiment, the mutated exon is exon 1, the target intron is intron 19, 22, 23, or 24, and the coding domain includes a functional ABCA4 exon 1. In one embodiment, exon 1 or exon 2 is mutated, the target intron is intron 19, 22, 23, or 24, and the coding domain includes functional ABCA4 exons 1 and 2. In one embodiment, one of exons 1, 2, and 3 is mutated, the target intron is intron 19, 22, 23, or 24, and the coding domain includes functional ABCA4 exons 1-3. In one embodiment, one of exons 1, 2, 3, and 4 is mutated, the target intron is intron 19, 22, 23, or 24, and the coding domain includes functional ABCA4 exons 1-4. In one embodiment, one of exons 1, 2, 3, 4, and 5 is mutated, the target intron is intron 19, 22, 23, or 24, and the coding domain includes functional ABCA4 exons 1-5. In one embodiment, one of exons 1, 2, 3, 4, 5, and 6 is mutated, the target intron is intron 19, 22, 23, or 24, and the coding domain includes functional ABCA4 exons 1-6. In one embodiment, one of exons 1, 2, 3, 4, 5, 6, or 7 is mutated, the target intron is intron 19, 22, 23, or 24, and the coding domain includes functional ABCA4 exons 1-7. In one embodiment, one of exons 1, 2, 3, 4, 5, 6, 7, or 8 is mutated, the target intron is intron 19, 22, 23, or 24, and the coding domain includes functional ABCA4 exons 1-8. In one embodiment, one of exons 1, 2, 3, 4, 5, 6, 7, 8, or 9 is mutated, the target intron is intron 19, 22, 23, or 24, and the coding domain includes functional ABCA4 exons 1-9. In one embodiment, one of exons 1, 2, 3, 4, 5, 6, 7, 8, 9, or 10 is mutated, the target intron is intron 19, 22, 23, or 24, and the coding domain includes functional ABCA4 exons 1-10. In one embodiment, one of exons 1, 2, 3, 4, 5, 6, 7, 8, 9, 10, or 11 is mutated, the target intron is intron 19, 22, 23, or 24, and the coding domain includes functional ABCA4 exons 1-11. In one embodiment, one of exons 1, 2, 3, 4, 5, 6, 7, 8, 9, 10, 11, or 12 is mutated, the target intron is intron 19, 22, 23, or 24, and the coding domain includes functional ABCA4 exons 1-12. In one embodiment, one of exons 1, 2, 3, 4, 5, 6, 7, 8, 9, 10, 11, 12, or 13 is mutated, the target intron is intron 19, 22, 23, or 24, and the coding domain includes functional ABCA4 exons 1-13. In one embodiment, one of exons 1, 2, 3, 4, 5, 6, 7, 8, 9, 10, 11, 12, or 13 is mutated, the target intron is intron 19, 22, 23, or 24, and the coding domain includes functional ABCA4 exons 1-13. In one embodiment, one of exons 1, 2, 3, 4, 5, 6, 7, 8, 9, 10, 11, 12, 13, or 14 is mutated, the target intron is intron 19, 22, 23, or 24, and the coding domain includes functional ABCA4 exons 1-14. In one embodiment, one of exons 1, 2, 3, 4, 5, 6, 7, 8, 9, 10, 11, 12, 13, 14, or 15 is mutated, the target intron is intron 19, 22, 23, or 24, and the coding domain includes functional ABCA4 exons 1-15. In one embodiment, one of exons 1, 2, 3, 4, 5, 6, 7, 8, 9, 10, 11, 12, 13, 14, 15, or 16 is mutated, the target intron is intron 19, 22, 23, or 24, and the coding domain includes functional ABCA4 exons 1-16. In one embodiment, one of exons 1, 2, 3, 4, 5, 6, 7, 8, 9, 10, 11, 12, 13, 14, 15, 16, or 17 is mutated, the target intron is intron 19, 22, 23, or 24, and the coding domain includes functional ABCA4 exons 1-17. In one embodiment, one of exons 1, 2, 3, 4, 5, 6, 7, 8, 9, 10, 11, 12, 13, 14, 15, 16, 17, or 18 is mutated, the target intron is intron 19, 22, 23, or 24, and the coding domain includes functional ABCA4 exons 1-18. In one embodiment, one of exons 1, 2, 3, 4, 5, 6, 7, 8, 9, 10, 11, 12, 13, 14, 15, 16, 17, 18, or 19 is mutated, the target intron is intron 19, 22, 23, or 24, and the coding domain includes functional ABCA4 exons 1-19. In one embodiment, one of exons 1, 2, 3, 4, 5, 6, 7, 8, 9, 10, 11, 12, 13, 14, 15, 16, 17, 18, 19, or 20 is mutated, the target intron is intron 22, 23, or 24, and the coding domain includes functional ABCA4 exons 1-20. In one embodiment, one of exons 1, 2, 3, 4, 5, 6, 7, 8, 9, 10, 11, 12, 13, 14, 15, 16, 17, 18, 19, 20, or 21 is mutated, the target intron is intron 22, 23, or 24, and the coding domain includes functional ABCA4 exons 1-21. In one embodiment, one of exons 1, 2, 3, 4, 5, 6, 7, 8, 9, 10, 11, 12, 13, 14, 15, 16, 17, 18, 19, 20, 21, or 22 is mutated, the target intron is intron 22, 23, or 24, and the coding domain includes functional ABCA4 exons 1-22. In one embodiment, one of exons 1, 2, 3, 4, 5, 6, 7, 8, 9, 10, 11, 12, 13, 14, 15, 16, 17, 18, 19, 20, 21, 22, or 23 is mutated, the target intron is intron 23 or 24, and the coding domain includes functional ABCA4 exons 1-23. In one embodiment, one of exons 1, 2, 3, 4, 5, 6, 7, 8, 9, 10, 11, 12, 13, 14, 15, 16, 17, 18, 19, 20, 21, 22, 23, or 24 is mutated, the target intron is intron 24, and the coding domain includes functional ABCA4 exons 1-24.

Alternatively, in instances in which a mutation is on the 3′ portion of the target gene, a 3′ trans-splicing molecule is selected to correct the mutation, e.g., without regard to the location of the mutation on the other allele. In one embodiment, one of exons 23, 24, 25, 26, 27, 28, 29, 30, 31, 32, 33, 34, 35, 36, 37, 38, 39, 40, 41, 42, 43, 44, 45, 46, 47, 48, 49, or 50 is mutated, the target intron is intron 22, and the coding domain includes functional ABCA4 exons 23-50. In one embodiment, one of exons 24, 25, 26, 27, 28, 29, 30, 31, 32, 33, 34, 35, 36, 37, 38, 39, 40, 41, 42, 43, 44, 45, 46, 47, 48, 49, or 50 is mutated, the target intron is intron 22 or 23, and the coding domain includes functional ABCA4 exons 24-50. In one embodiment, one of exons 25, 26, 27, 28, 29, 30, 31, 32, 33, 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 25-50. In one embodiment, one of exons 26, 27, 28, 29, 30, 31, 32, 33, 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 26-50. In one embodiment, one of exons 27, 28, 29, 30, 31, 32, 33, 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 27-50. In one embodiment, one of exons 28, 29, 30, 31, 32, 33, 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 28-50. In one embodiment, one of exons 29, 30, 31, 32, 33, 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 29-50. In one embodiment, one of exons 30, 31, 32, 33, 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 30-50. In one embodiment, one of exons 31, 32, 33, 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 31-50. In one embodiment, one of exons 32, 33, 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 32-50. In one embodiment, one of exons 33, 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 33-50. In one embodiment, one of exons 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 34-50. In one embodiment, one of exons 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 35-50. In one embodiment, one of exons 36, 37, 38, 39, 40, 41, 42, 43, 44, 45, 46, 47, 48, 49, or 50 is mutated, the target intron is intron 22, 23, or 24, and the coding domain includes functional ABCA4 exons 36-50. In one embodiment, one of exons 37, 38, 39, 40, 41, 42, 43, 44, 45, 46, 47, 48, 49, or 50 is mutated, the target intron is intron 22, 23, or 24, and the coding domain includes functional ABCA4 exons 37-50. In one embodiment, one of exons 38, 39, 40, 41, 42, 43, 44, 45, 46, 47, 48, 49, or 50 is mutated, the target intron is intron 22, 23, or 24, and the coding domain includes functional ABCA4 exons 38-50. In one embodiment, one of exons 39, 40, 41, 42, 43, 44, 45, 46, 47, 48, 49, or 50 is mutated, the target intron is intron 22, 23, or 24, and the coding domain includes functional ABCA4 exons 39-50. In one embodiment, one of exons 40, 41, 42, 43, 44, 45, 46, 47, 48, 49, or 50 is mutated, the target intron is intron 22, 23, or 24, and the coding domain includes functional ABCA4 exons 40-50. In one embodiment, one of exons 41, 42, 43, 44, 45, 46, 47, 48, 49, or 50 is mutated, the target intron is intron 22, 23, or 24, and the coding domain includes functional ABCA4 exons 41-50. In one embodiment, one of exons 42, 43, 44, 45, 46, 47, 48, 49, or 50 is mutated, the target intron is intron 22, 23, or 24, and the coding domain includes functional ABCA4 exons 42-50. In one embodiment, one of exons 43, 44, 45, 46, 47, 48, 49, or 50 is mutated, the target intron is intron 22, 23, or 24, and the coding domain includes functional ABCA4 exons 43-50. In one embodiment, one of exons 44, 45, 46, 47, 48, 49, or 50 is mutated, the target intron is intron 22, 23, or 24, and the coding domain includes functional ABCA4 exons 44-50. In one embodiment, one of exons 45, 46, 47, 48, 49, or 50 is mutated, the target intron is intron 22, 23, or 24, and the coding domain includes functional ABCA4 exons 45-50. In one embodiment, one of exons 46, 47, 48, 49, or 50 is mutated, the target intron is intron 22, 23, or 24, and the coding domain includes functional ABCA4 exons 46-50. In one embodiment, one of exons 47, 48, 49, or 50 is mutated, the target intron is intron 22, 23, or 24, and the coding domain includes functional ABCA4 exons 47-50. In one embodiment, one of exons 48, 49, or 50 is mutated, the target intron is intron 22, 23, or 24, and the coding domain includes functional ABCA4 exons 48-50. In one embodiment, one of exons 49 or 50 is mutated, the target intron is intron 22, 23, or 24, and the coding domain includes functional ABCA4 exons 49 or 50. In one embodiment, exon 50 is mutated, the target intron is intron 22, 23, or 24, and the coding domain includes functional ABCA4 exon 50.

### Single Trans-Splicing Molecules for Correcting Multiple Mutations

Methods of the invention include selecting a single trans-splicing molecule based on the location of a mutation in ABCA4 in each allele of the subject, when two mutations are either on a 5′ portion of the gene or the 3′ portion of the gene, such that a single trans-splicing molecule capable of being packaged in an AAV vector is capable of spanning both mutations, thereby correcting both mutations.

For example, in instances in which both mutations occur on the 5′ portion of the target gene, a 5′ trans-splicing molecule is selected to correct both mutations. In one embodiment, the mutated exon is exon 1 (i.e., both mutations are in exon 1), the target intron is intron 19, 22, 23, or 24, and the coding domain includes a functional ABCA4 exon 1. In one embodiment, exon 1 and/or exon 2 are mutated, the target intron is intron 19, 22, 23, or 24, and the coding domain includes functional ABCA4 exons 1 and 2. In one embodiment, one or two of exons 1, 2, and 3 is mutated, the target intron is intron 19, 22, 23, or 24, and the coding domain includes functional ABCA4 exons 1-3. In one embodiment, one or two of exons 1, 2, 3, and 4 is mutated, the target intron is intron 19, 22, 23, or 24, and the coding domain includes functional ABCA4 exons 1-4. In one embodiment, one or two of exons 1, 2, 3, 4, and 5 is mutated, the target intron is intron 19, 22, 23, or 24, and the coding domain includes functional ABCA4 exons 1-5. In one embodiment, one or two of exons 1, 2, 3, 4, 5, and 6 is mutated, the target intron is intron 19, 22, 23, or 24, and the coding domain includes functional ABCA4 exons 1-6. In one embodiment, one or two of exons 1, 2, 3, 4, 5, 6, or 7 is mutated, the target intron is intron 19, 22, 23, or 24, and the coding domain includes functional ABCA4 exons 1-7. In one embodiment, one or two of exons 1, 2, 3, 4, 5, 6, 7, or 8 is mutated, the target intron is intron 19, 22, 23, or 24, and the coding domain includes functional ABCA4 exons 1-8. In one embodiment, one or two of exons 1, 2, 3, 4, 5, 6, 7, 8, or 9 is mutated, the target intron is intron 19, 22, 23, or 24, and the coding domain includes functional ABCA4 exons 1-9. In one embodiment, one or two of exons 1, 2, 3, 4, 5, 6, 7, 8, 9, or 10 is mutated, the target intron is intron 19, 22, 23, or 24, and the coding domain includes functional ABCA4 exons 1-10. In one embodiment, one or two of exons 1, 2, 3, 4, 5, 6, 7, 8, 9, 10, or 11 is mutated, the target intron is intron 19, 22, 23, or 24, and the coding domain includes functional ABCA4 exons 1-11. In one embodiment, one or two of exons 1, 2, 3, 4, 5, 6, 7, 8, 9, 10, 11, or 12 is mutated, the target intron is intron 19, 22, 23, or 24, and the coding domain includes functional ABCA4 exons 1-12. In one embodiment, one or two of exons 1, 2, 3, 4, 5, 6, 7, 8, 9, 10, 11, 12, or 13 is mutated, the target intron is intron 19, 22, 23, or 24, and the coding domain includes functional ABCA4 exons 1-13. In ono embodiment, one or two of exons 1, 2, 3, 4, 5, 6, 7, 8, 9, 10, 11, 12, or 13 is mutated, the target intron is intron 19, 22, 23, or 24, and the coding domain includes functional ABCA4 exons 1-13. In one embodiment, one or two of exons 1, 2, 3, 4, 5, 6, 7, 8, 9, 10, 11, 12, 13, or 14 is mutated, the target intron is intron 19, 22, 23, or 24, and the coding domain includes functional ABCA4 exons 1-14. In one embodiment, one or two of exons 1, 2, 3, 4, 5, 6, 7, 8, 9, 10, 11, 12, 13, 14, or 15 is mutated, the target intron is intron 19, 22, 23, or 24, and the coding domain includes functional ABCA4 exons 1-15. In one embodiment, one or two of exons 1, 2, 3, 4, 5, 6, 7, 8, 9, 10, 11, 12, 13, 14, 15, or 16 is mutated, the target intron is intron 19, 22, 23, or 24, and the coding domain includes functional ABCA4 exons 1-16. In one embodiment, one or two of exons 1, 2, 3, 4, 5, 6, 7, 8, 9, 10, 11, 12, 13, 14, 15, 16, or 17 is mutated, the target intron is intron 19, 22, 23, or 24, and the coding domain includes functional ABCA4 exons 1-17. In one embodiment, one or two of exons 1, 2, 3, 4, 5, 6, 7, 8, 9, 10, 11, 12, 13, 14, 15, 16, 17, or 18 is mutated, the target intron is intron 19, 22, 23, or 24, and the coding domain includes functional ABCA4 exons 1-18. In one embodiment, one or two of exons 1, 2, 3, 4, 5, 6, 7, 8, 9, 10, 11, 12, 13, 14, 15, 16, 17, 18, or 19 is mutated, the target intron is intron 19, 22, 23, or 24, and the coding domain includes functional ABCA4 exons 1-19. In one embodiment, one or two of exons 1, 2, 3, 4, 5, 6, 7, 8, 9, 10, 11, 12, 13, 14, 15, 16, 17, 18, 19, or 20 is mutated, the target intron is intron 22, 23, or 24, and the coding domain includes functional ABCA4 exons 1-20. In one embodiment, one or two of exons 1, 2, 3, 4, 5, 6, 7, 8, 9, 10, 11, 12, 13, 14, 15, 16, 17, 18, 19, 20, or 21 is mutated, the target intron is intron 22, 23, or 24, and the coding domain includes functional ABCA4 exons 1-21. In one embodiment, one or two of exons 1, 2, 3, 4, 5, 6, 7, 8, 9, 10, 11, 12, 13, 14, 15, 16, 17, 18, 19, 20, 21, or 22 is mutated, the target intron is intron 22, 23, or 24, and the coding domain includes functional ABCA4 exons 1-22. In one embodiment, one or two of exons 1, 2, 3, 4, 5, 6, 7, 8, 9, 10, 11, 12, 13, 14, 15, 16, 17, 18, 19, 20, 21, 22, or 23 is mutated, the target intron is intron 23 or 24, and the coding domain includes functional ABCA4 exons 1-23. In one embodiment, one or two of exons 1, 2, 3, 4, 5, 6, 7, 8, 9, 10, 11, 12, 13, 14, 15, 16, 17, 18, 19, 20, 21, 22, 23, or 24 is mutated, the target intron is intron 24, and the coding domain includes functional ABCA4 exons 1-24.

Alternatively, in instances in which both mutations occur on the 3′ portion of the target gene, a 3′ trans-splicing molecule is selected to correct both mutations. In one embodiment, one or two of exons 23, 24, 25, 26, 27, 28, 29, 30, 31, 32, 33, 34, 35, 36, 37, 38, 39, 40, 41, 42, 43, 44, 45, 46, 47, 48, 49, or 50 is mutated, the target intron is intron 22, and the coding domain includes functional ABCA4 exons 23-50. In one embodiment, one or two of exons 24, 25, 26, 27, 28, 29, 30, 31, 32, 33, 34, 35, 36, 37, 38, 39, 40, 41, 42, 43, 44, 45, 46, 47, 48, 49, or 50 is mutated, the target intron is intron 22 or 23, and the coding domain includes functional ABCA4 exons 24-50. In one embodiment, one or two of exons 25, 26, 27, 28, 29, 30, 31, 32, 33, 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 25-50. In one embodiment, one or two of exons 26, 27, 28, 29, 30, 31, 32, 33, 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 26-50. In one embodiment, one or two of exons 27, 28, 29, 30, 31, 32, 33, 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 27-50. In one embodiment, one or two of exons 28, 29, 30, 31, 32, 33, 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 28-50. In one embodiment, one or two of exons 29, 30, 31, 32, 33, 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 29-50. In one embodiment, one or two of exons 30, 31, 32, 33, 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 30-50. In one embodiment, one or two of exons 31, 32, 33, 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 31-50. In one embodiment, one or two of exons 32, 33, 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 32-50. In one embodiment, one or two of exons 33, 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 33-50. In one embodiment, one or two of exons 34, 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 34-50. In one embodiment, one or two of exons 35, 36, 37, 38, 39, 40, 41, 42, 43, 44, 45, 46, 47, 48, 49, or 50 is mutated, the target intron is intron 22, 23, or 24, and the coding domain includes functional ABCA4 exons 35-50. In one embodiment, one or two of exons 36, 37, 38, 39, 40, 41, 42, 43, 44, 45, 46, 47, 48, 49, or 50 is mutated, the target intron is intron 22, 23, or 24, and the coding domain includes functional ABCA4 exons 36-50. In one embodiment, one or two of exons 37, 38, 39, 40, 41, 42, 43, 44, 45, 46, 47, 48, 49, or 50 is mutated, the target intron is intron 22, 23, or 24, and the coding domain includes functional ABCA4 exons 37-50. In one embodiment, one or two of exons 38, 39, 40, 41, 42, 43, 44, 45, 46, 47, 48, 49, or 50 is mutated, the target intron is intron 22, 23, or 24, and the coding domain includes functional ABCA4 exons 38-50. In one embodiment, one or two of exons 39, 40, 41, 42, 43, 44, 45, 46, 47, 48, 49, or 50 is mutated, the target intron is intron 22, 23, or 24, and the coding domain includes functional ABCA4 exons 39-50. In one embodiment, one or two of exons 40, 41, 42, 43, 44, 45, 46, 47, 48, 49, or 50 is mutated, the target intron is intron 22, 23, or 24, and the coding domain includes functional ABCA4 exons 40-50. In one embodiment, one or two of exons 41, 42, 43, 44, 45, 46, 47, 48, 49, or 50 is mutated, the target intron is intron 22, 23, or 24, and the coding domain includes functional ABCA4 exons 41-50. In one embodiment, one or two of exons 42, 43, 44, 45, 46, 47, 48, 49, or 50 is mutated, the target intron is intron 22, 23, or 24, and the coding domain includes functional ABCA4 exons 42-50. In one embodiment, one or two of exons 43, 44, 45, 46, 47, 48, 49, or 50 is mutated, the target intron is intron 22, 23, or 24, and the coding domain includes functional ABCA4 exons 43-50. In one embodiment, one or two of exons 44, 45, 46, 47, 48, 49, or 50 is mutated, the target intron is intron 22, 23, or 24, and the coding domain includes functional ABCA4 exons 44-50. In one embodiment, one or two of exons 45, 46, 47, 48, 49, or 50 is mutated, the target intron is intron 22, 23, or 24, and the coding domain includes functional ABCA4 exons 45-50. In one embodiment, one or two of exons 46, 47, 48, 49, or 50 is mutated, the target intron is intron 22, 23, or 24, and the coding domain includes functional ABCA4 exons 46-50. In one embodiment, one or two of exons 47, 48, 49, or 50 is mutated, the target intron is intron 22, 23, or 24, and the coding domain includes functional ABCA4 exons 47-50. In one embodiment, one or two of exons 48, 49, or 50 is mutated, the target intron is intron 22, 23, or 24, and the coding domain includes functional ABCA4 exons 48-50. In one embodiment, one or two of exons 49 or 50 is mutated, the target intron is intron 22, 23, or 24, and the coding domain includes functional ABCA4 exons 49 or 50. In one embodiment, exon 50 is mutated, the target intron is intron 22, 23, or 24, and the coding domain includes functional ABCA4 exon 50.

### Two Trans-Splicing Molecules for Correcting Multiple Mutations

Additionally, provided herein are methods of correcting multiple mutations within an ABCA4 gene using two trans-splicing molecules—a 5′ trans-splicing molecule and a 3′ trans-splicing molecule. In some embodiments, the entire ABCA4 gene is replaced upon binding of both trans-splicing molecules, for example, where the 5′ trans-splicing molecule and the 3′ trans-splicing molecule bind the same target ABCA4 intron and replace the exons upstream and downstream, respectively, of the target intron.

For example, in some embodiments of the invention, a 5′ trans-splicing molecule and a 3′ trans-splicing molecule each bind target ABCA4 intron 22; the 5′ trans-splicing molecule replaces endogenous exons 1-22 with functional exons 1-22; and the 3′ trans-splicing molecule replaces endogenous exons 23-50 with functional exons 23-50. In other embodiments, a 5′ trans-splicing molecule and a 3′ trans-splicing molecule each bind target ABCA4 intron 23; the 5′ trans-splicing molecule replaces endogenous exons 1-23 with functional exons 1-23; and the 3′ trans-splicing molecule replaces endogenous exons 24-50 with functional exons 24-50. In other embodiments, a 5′ trans-splicing molecule and a 3′ trans-splicing molecule each bind target ABCA4 intron 24; the 5′ trans-splicing molecule replaces endogenous exons 1-24 with functional exons 1-24; and the 3′ trans-splicing molecule replaces endogenous exons 25-50 with functional exons 25-50. Any of the aforementioned combinations of 5′ and 3′ trans-splicing molecules can be included in a pharmaceutical composition (e.g., a single pharmaceutical composition including both molecules, either pre-prepared or admixed prior to administration, e.g., as part of a kit).

### Dosing, Monitoring, and Combination Therapies

An effective concentration of a recombinant adeno-associated virus carrying a trans-splicing molecule as described herein ranges between about 10⁸ and 10¹³ vector genomes per milliliter (vg/mL). The rAAV infectious units are measured as described in McLaughlin et al., J. Virol. 1988, 62:1963. In one embodiment, the concentration ranges between 10⁹ and 10¹³ vg/mL. In another embodiment, the effective concentration is about 1.5×10¹¹ vg/mL. In one embodiment, the effective concentration is about 1.5×10¹⁰ vg/mL. In another embodiment, the effective concentration is about 2.8×10¹¹ vg/mL. In another embodiment, the effective concentration is about 5×10¹¹ vg/mL. In yet another embodiment, the effective concentration is about 1.5×10¹² vg/mL. In another embodiment, the effective concentration is about 1.5×10¹³ vg/mL.

It is desirable that the lowest effective dosage (total genome copies delivered) of virus be utilized in order to reduce the risk of undesirable effects, such as toxicity, and other issues related to administration to the eye, e.g., retinal dysplasia and detachment. An effective dosage of a recombinant adeno-associated virus carrying a trans-splicing molecule as described herein ranges between about 10⁸ and 10¹³ vector genomes (vg) per dose (i.e., per injection). In one embodiment, the dosage ranges between 10⁹ and 10¹³ vg. In another embodiment, the effective dosage is about 1.5×10¹¹ vg. In another embodiment, the effective dosage is about 5×10¹¹ vg. In one embodiment, the effective dosage is about 1.5×10¹⁰ vg. In another embodiment, the effective dosage is about 2.8×10¹¹ vg. In yet another embodiment, the effective dosage is about 1.5×10¹² vg. In another embodiment, the effective concentration is about 1.5×10¹³ vg. Still other dosages in these ranges or in other units may be selected by the attending physician, taking into account the physical state of the subject being treated, including the age of the subject; the composition being administered, and the particular disorder; the targeted cell and the degree to which the disorder, if progressive, has developed.

The composition may be delivered in a volume of from about 50 μL to about 1 mL, including all numbers within the range, depending on the size of the area to be treated, the viral titer used, the route of administration, and the desired effect of the method. In one embodiment, the volume is about 50 μL. In another embodiment, the volume is about 70 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 350 μL. In another embodiment, the volume is about 400 μL In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1,000 μL.

In one embodiment, the volume and concentration of the rAAV composition is selected so that only certain anatomical regions having target cells are impacted. In another embodiment, the volume and/or concentration of the rAAV composition is a greater amount, in order reach larger portions of the eye. Similarly dosages are adjusted for administration to other organs.

In another embodiment, the invention provides a method to prevent, or arrest photoreceptor function loss, or increase photoreceptor function in the subject. The composition may be administered before or after disease onset. For example, photoreceptor function may be assessed using the functional studies, e.g., ERG or perimetry, which are conventional in the art. As used herein “photoreceptor function loss” means a decrease in photoreceptor function as compared to a normal, non-diseased eye or the same eye at an earlier time point. As used herein, “increase photoreceptor function” means to improve the function of the photoreceptors or increase the number or percentage of functional photoreceptors as compared to a diseased eye (having the same ocular disease), the same eye at an earlier time point, a non-treated portion of the same eye, or the contralateral eye of the same subject.

For each of the described methods, the treatment may be used to prevent the occurrence of further damage or to rescue tissue having mild or advanced disease. As used herein, the term “rescue” means to prevent progression of the disease, prevent spread of damage to uninjured cells or to improve damage in injured cells.

Thus, in one embodiment, the composition is administered before disease onset. In another embodiment, the composition is administered prior to the development of symptoms. In another embodiment, the composition is administered after development of symptoms. In yet another embodiment, the composition is administered when less than 90% of the target cells are functioning or remaining, e.g., as compared to a reference tissue. In yet another embodiment, the composition is administered when more than 10% of the target cells are functioning or remaining, e.g., as compared to a reference tissue. In yet another embodiment, the composition is administered when more than 20% of the target cells are functioning or remaining. In yet another embodiment, the composition is administered when more than 30% of the target cells are functioning or remaining.

In yet another embodiment, any of the above described methods is performed in combination with another, or secondary, therapy. The therapy may be any now known, or as yet unknown, therapy which helps prevent, arrest or ameliorate these mutations or defects or any of the effects associated therewith. The secondary therapy can be administered before, concurrent with, or after administration of the trans-splicing molecules described above. In one embodiment, a secondary therapy involves non-specific approaches for maintaining the health of the retinal cells, such as administration of neurotrophic factors, anti-oxidants, anti-apoptotic agents. The non-specific approaches are achieved through injection of proteins, recombinant DNA, recombinant viral vectors, stem cells, fetal tissue, or genetically modified cells. The latter could include genetically modified cells that are encapsulated.

In another embodiment, the method includes performing functional and imaging studies to determine the efficacy of the treatment. These studios include electroretinography (ERG) and in vivo retinal imaging, as described in U.S. Pat. No. 8,147,823; in International Patent Publication Nos. WO 2014/011210 or WO 2014/124282, incorporated herein by reference. In addition visual field studies, perimetry and microperimetry, mobility testing, visual acuity, and/or color vision testing may be performed.

In certain embodiments, it is desirable to perform non-invasive retinal imaging and functional studies to identify areas of retained photoreceptors to be targeted for therapy. In these embodiments, clinical diagnostic tests are employed to determine the precise location(s) for one or more subretinal injection(s). These tests may include ERG, perimetry, topographical mapping of the layers of the retina and measurement of the thickness of its layers by means of confocal scanning laser ophthalmoscopy (cSLO) and optical coherence tomography (OCT), topographical mapping of cone density via adaptive optics (AO), functional eye exam, etc. In view of the imaging and functional studies, in some embodiments one or more injections are performed in the same eye in order to target different areas of retained photoreceptors.

For use in these methods, the volume and viral titer of each injection is determined individually, and may be the same or different from other injections performed in the same, or contralateral, eye. In another embodiment, a single, larger volume injection is made in order to treat the entire eye. The dosages, administrations, and regimens may be determined by the attending physician given the teachings of this disclosure.

### EXAMPLES

The invention is based, at least in part, on Applicant's discovery that particular introns of ABCA4, and specific regions within those introns, provide highly efficient binding sites for binding domains of trans-splicing molecules and efficiently mediate trans-splicing. Applicant has generated a series of mock trans-splicing molecules having 150-mer binding domains designed to hybridize to a corresponding series of 150-base pair binding site sequences of (i) ABCA4 introns of interest (introns 19 and 22-24) and (ii) CEP290 introns of interest (introns 26-30). Each binding domain in the ABCA4 series and the CEP290 series was designed to overlap by 140 nucleotides, enabling scanning of each intron by 10 nucleotides between each sequential test binding domain. Trans-splicing efficiency was quantified for each binding domain across each of ABCA4 introns 19 and 22-24 and CEP290 introns 26-30. ABCA4 screening is described in Example 1, and results are shown in FIGS. 1-8. CEP290 screening is described in Example 2, and results are shown in FIGS. 21-26.

### Example 1. ABCA4

This Example describes development of ABCA4 trans-splicing molecules, for example, by screening for effective binding sites within particular ABCA4 introns, developing an ABCA4 cell line for testing trans-splicing molecules, and testing various ABCA4 trans-splicing molecules for restoration of ABCA4 protein expression.

### Binding Site Screening

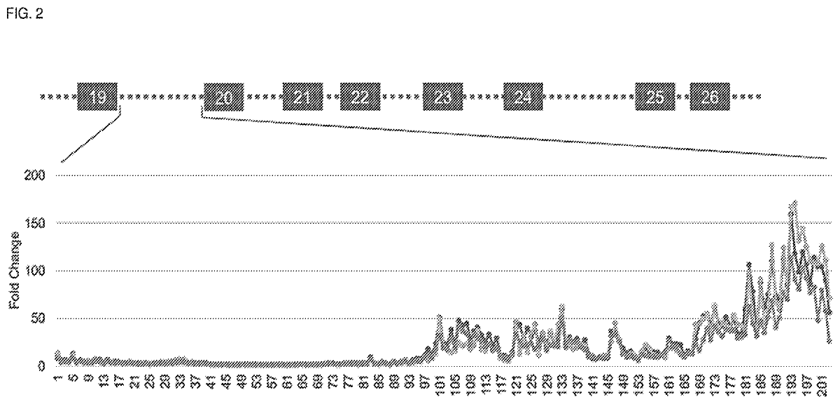

Screening of a series of binding domains configured to bind ABCA4 intron 19 (SEQ ID NO: 25) at sequential bindings sites revealed a region at the 3′ portion of ABCA4 intron 19 that was preferentially efficient at trans-splicing of a 5′ trans-splicing molecule—a region from nucleotides 990 to 2, 174 of intron 19 (FIG. 2). Binding sites within the range of nucleotides from 1,670 to 2,174, from 1,810 to 2,000, from 1,870 to 2,000, or from 1,920 to 2,000 were revealed as particularly highly efficient at mediating 5′ trans-splicing at intron 19.

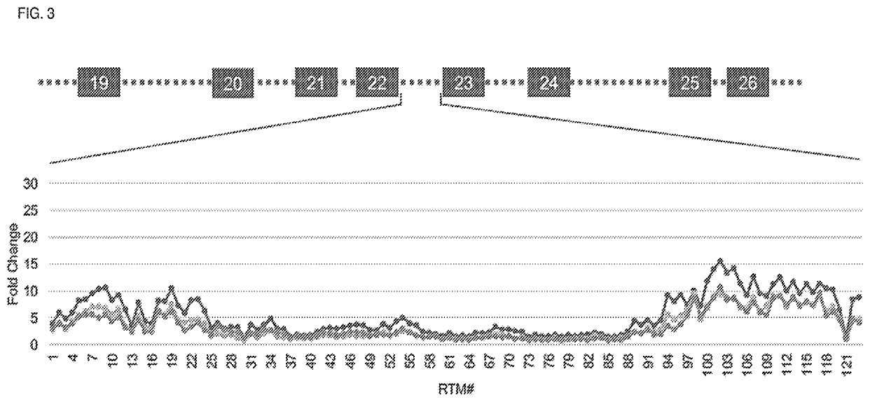

Suitable binding sites for 5′ trans-splicing molecules within intron 22 were similarly identified (FIG. 3). Binding sites within the ranges of nucleotides 1 to 150 or nucleotides 880 to 1,350 of intron 22 were especially efficient, relative to the remainder of the intron.

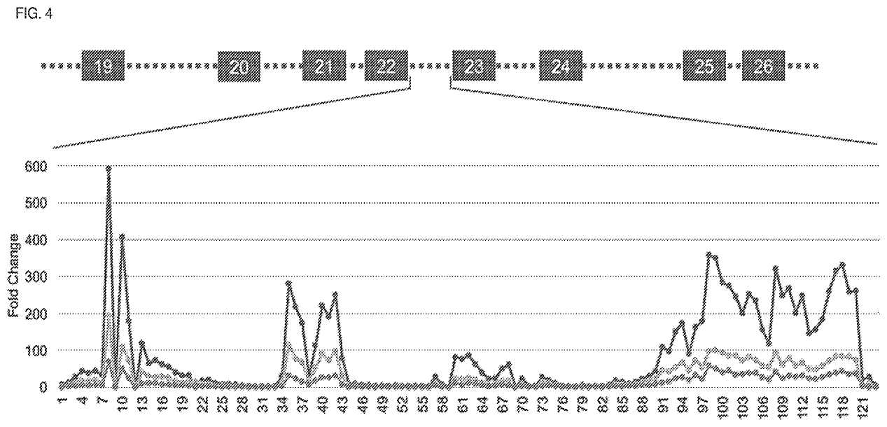

FIG. 4 shows results of an analogous screen for ABCA4 intron 22 (SEQ ID NO: 28) for a 3′ trans-splicing molecule. Binding sites having nucleotides 60 to 570, nucleotides 600 to 800, or nucleotides 900 to 1,350 were identified as preferentially suitable for trans-splicing of a 3′ trans-splicing molecule. In particular, binding domains targeted to binding sites within the range of nucleotides 70 to 250 were highly efficient at 3′ trans-splicing.

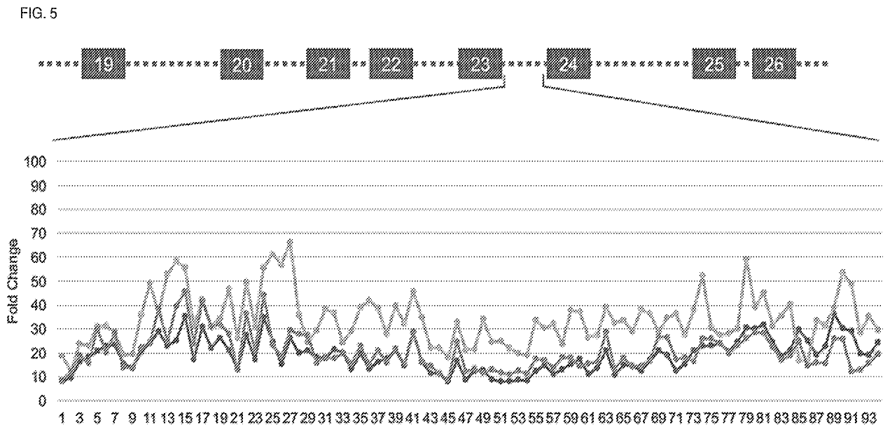

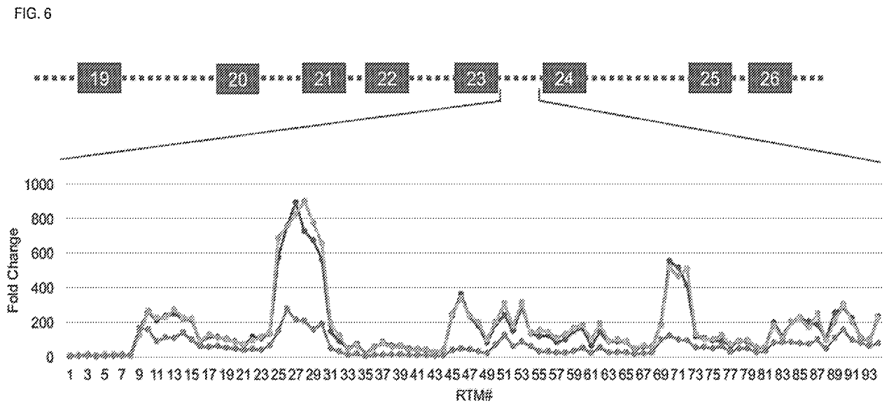

Within intron 23, relatively efficient binding sites for 5′ trans-splicing molecules were identified as those within the ranges of nucleotides 80 to 570 or nucleotides 720 to 1,081 of SEQ ID NO: 29 (FIG. 5). For 3′ trans-splicing molecules, binding sites with particularly good efficiency included those within nucleotides 80 to 1,081 of SEQ ID NO: 29 (e.g., nucleotides 230 to 1,081 of SEQ ID NO: 29, nucleotides 250 to 400 of SEQ ID NO: 29, or nucleotides 690 to 850 of SEQ ID NO: 29), as shown in FIG. 6.

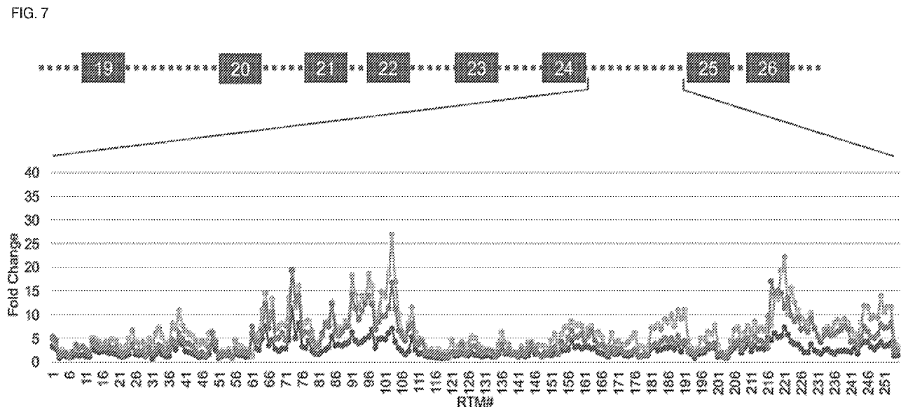

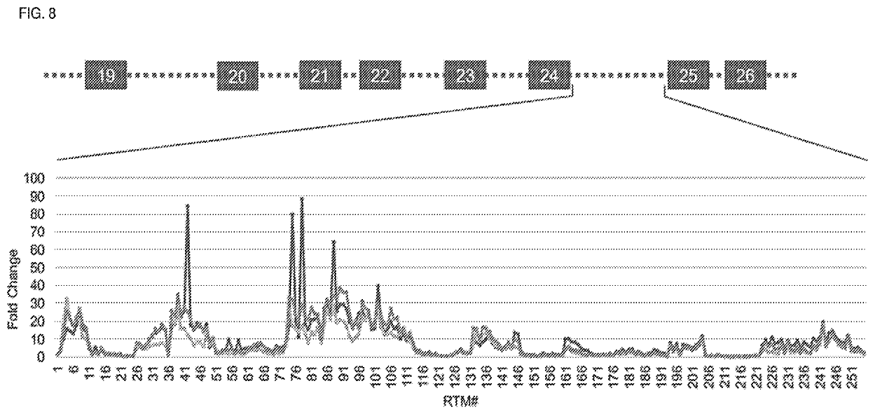

An analogous screening at intron 24 of ABCA4 (SEQ ID NO: 30) revealed that binding sites within the ranges of nucleotides 600 to 1,250 or nucleotides 1,490 to 2,660 were efficient at 5′ trans-splicing (FIG. 7). In particular, binding sites within the range of nucleotides 1,000 to 1,200 exhibited the greatest 5′ trans-splicing efficiency. FIG. 8 shows the results of a screening of 3′ trans-splicing efficiency, which revealed that binding sites within the range of nucleotides 1 to 250, nucleotides 300 to 2,000, or nucleotides 2,200 to 2,692 (in particular, binding sites within the range of nucleotides or nucleotides 750 to 1,110) were most efficient.

### ABCA4 Cell Lines

First, cell lines expressing ABCA4 were generated. The ABCA4 gene is only known to be expressed in living photoreceptors in the retina, and full-length ABCA4 pre-mRNA and protein are not generally detectable in cultured cells in vitro. Therefore, to test trans-splicing strategies for ABCA4, cells were engineered to express ABCA4 from its native genomic locus on chromosome 1 (1p22.1). Two strategies were pursued. In the first case, stable cell lines were derived to express site-specific (upstream of the ABCA4 transcriptional start site) DNA-binding TALENs that were fused to the VP64 viral trans-activator. In the second case, a constitutive eukaryotic promotor was directly inserted (using CRISPR/Cas9) into the genomic locus immediately upstream of the ABCA4 transcriptional start site. The results in both cases were stable cell lines that robustly expressed ABCA4 pre-mRNA and protein.

### TALEN Cell Lines

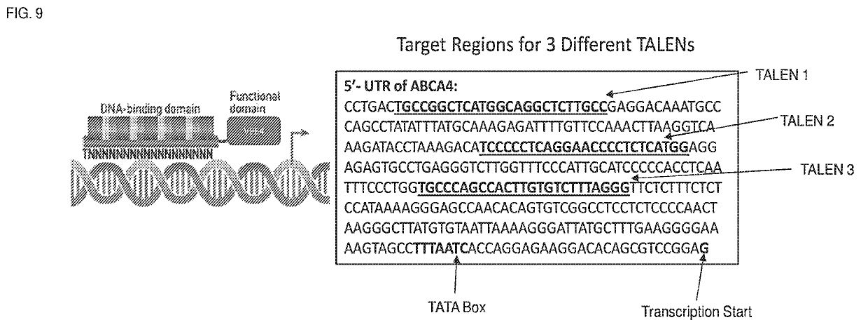

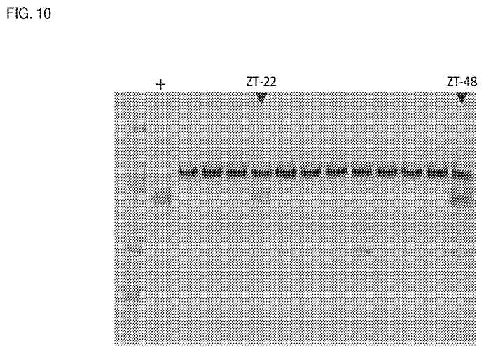

TALENs targeted to specific domains upstream of the ABCA4 transcriptional start site were designed and fused to a VP-64 trans-activator sequence (FIG. 9). This combination of three TALENs were transfected into 293 cells and stable single cell clones were derived. Two clones were shown to direct expression of ABCA4 protein (FIG. 10).

### CAG Promoter Cell Lines

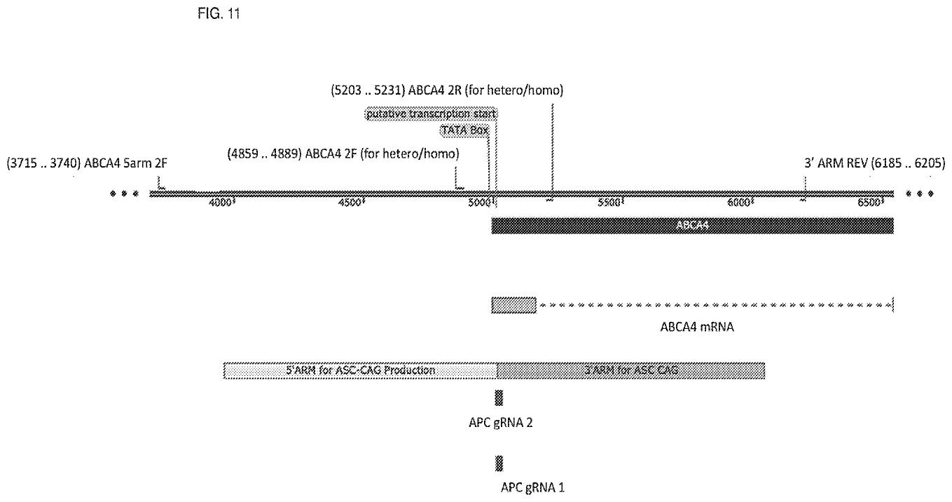

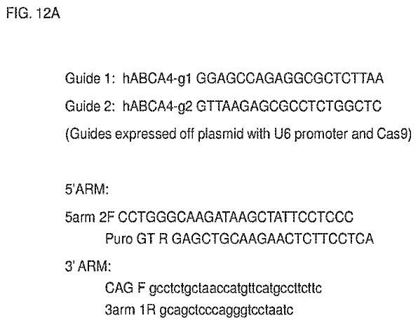

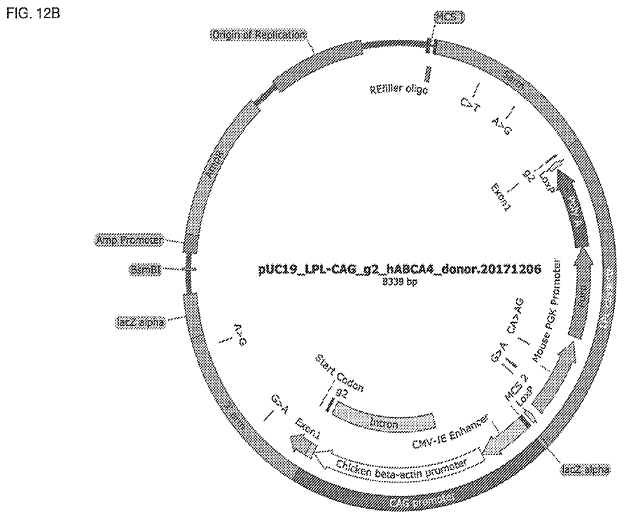

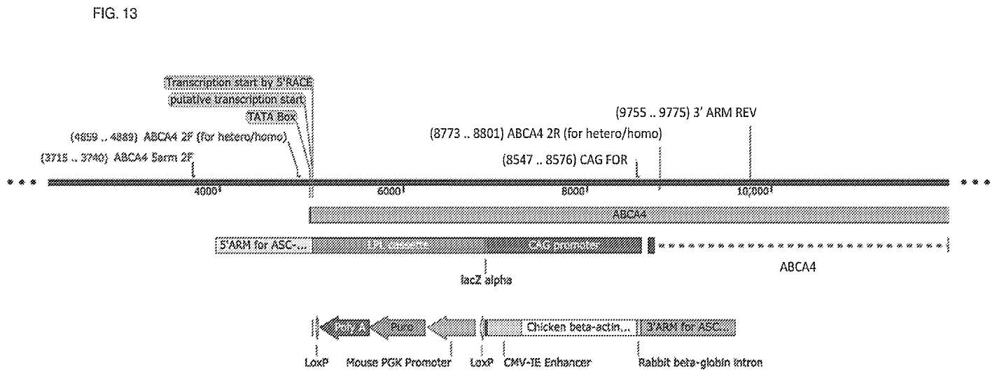

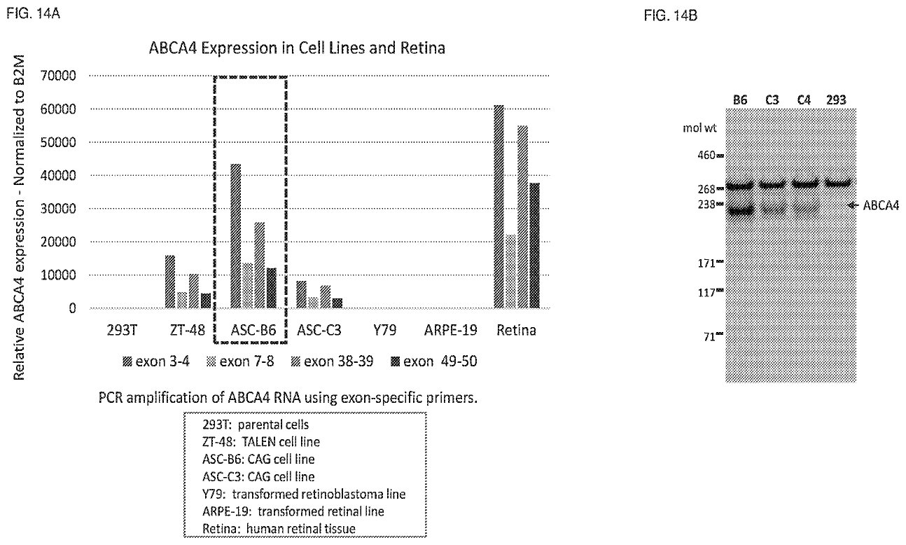

The general strategy for deriving CAG promoter cell lines is outlined in FIGS. 11-13. Site-specific guides (FIG. 12A) were designed to insert the CAG promoter and a puromycin selectable marker using homology arms (FIG. 12B). Puromycin resistant cells were cloned and analyzed by PCR for the desired insertion. Several clonal lines were selected for further analyses. RNA and protein expression for two lines (B6 and C3) are shown in FIGS. 14A and 14B. Both lines clearly contained the promoter insertion, as demonstrated by RNA and protein analyses.

### ABCA4 Knockout Cell Lines

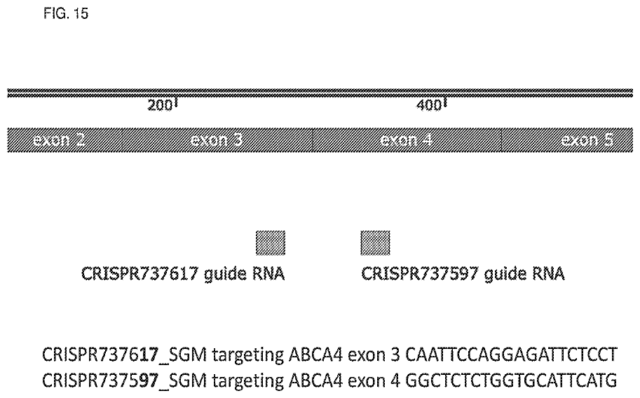

Once stable ABCA4 expression was established in cultured cells, knock-outs of ABCA4 expression were generated for testing of ABCA4 trans-splicing molecules designed to restore ABCA4 protein expression. In general, guide RNA and Cas9 protein were co-transfected in B6 cells (CAG-promotor knocked into ABCA4 locus and mediating ABCA4 expression). After nine days, a second transfection with guide RNA and Cas9 protein was performed. The basic design targeting exons 3 and 4 is shown in FIG. 15. Single cells were plated by limiting dilution and once grown, evaluated for protein expression of ABCA4 by Western blot.

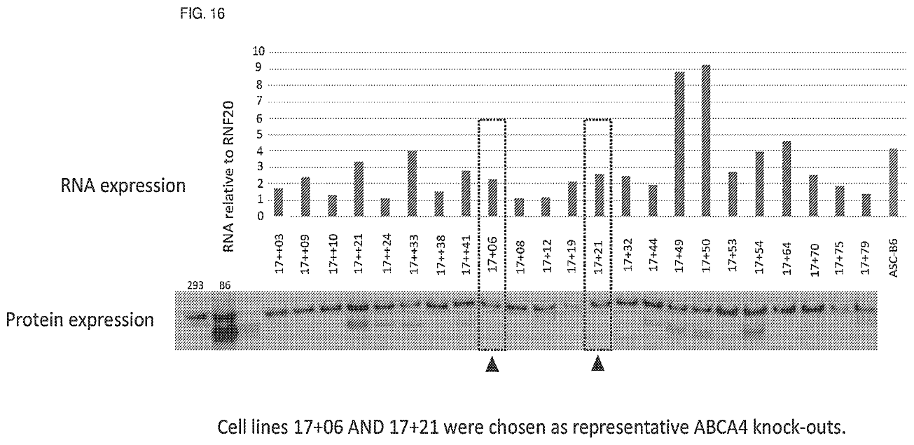

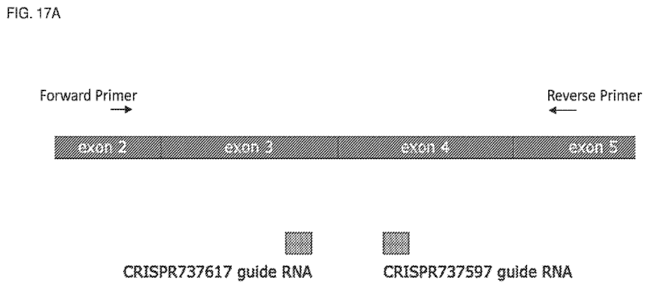

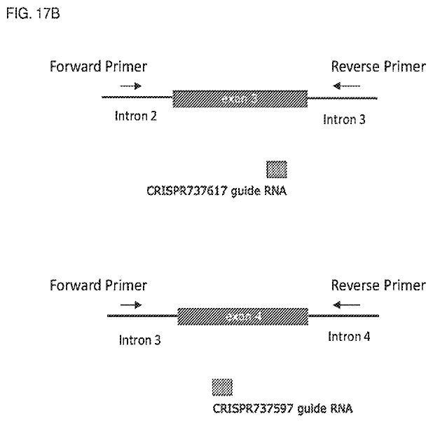

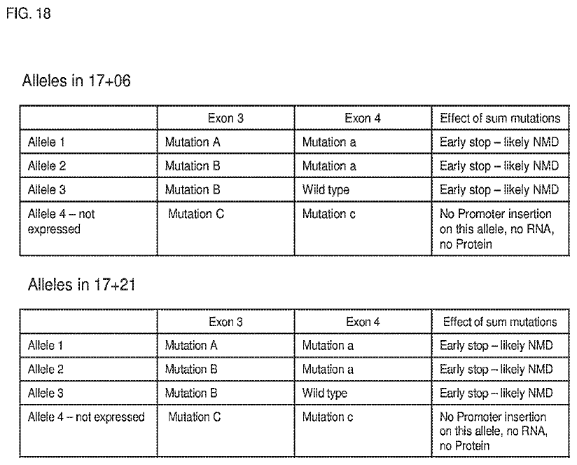

FIG. 16 shows the RNA and protein profiles of single cell clones derived after treatment with CRISPR/Cas9, as depicted in FIG. 15. There were varying degrees of RNA and protein ablation. Clones 17+06 and 17+21 were chosen because they exhibited complete ABCA4 protein knockout. Mutation analyses (FIGS. 17A-17B and 18) confirmed that exons 3 and 4 were targeted and interrupted.

### ABCA4 Trans-Splicing-Mediated Protein Restoration

Eight trans-splicing molecules were selected based on the high-throughput binding site screening described above. Methods and results of these studies are described below

### Methods

For Western blot assays, 17+06 or 17+21 cells were seeded at a density of 10⁶ cells per well in each well of twelve-well plates. Individual wells were transfected with 1 μg of plasmid (RTMₓ). At 48 hours, cells were harvested, and membrane preparations were processed for analysis by standard western blotting using Mem-PER Plus Membrane Protein Extraction Kit (Thermo Fisher 89842) according to the manufacturer's protocol with addition of 1× HALT™ Protease and Phosphatase Inhibitor Cocktail (Thermo Fisher 78440) in all buffers. RNA was also processed for analysis as described below. Membrane lysates were denatured with 4× Laemmli Sample Buffer (Biorad 161-0737) including 10% reducing agent TCEP 0.5 M (Sigma 646547) for 30 minutes at room temperature. Samples were run on NuPage Precast 3-8% Tris-Acetate gels (Thermo Fisher) and proteins were transferred using the iBlot 2 Mini PVDF Transfer Packs—run at 25V 10 minutes with iBlot 2. The primary antibody for ABCA4 was Abcam ab72955, rabbit polyclonal (@1:2500 dilution). The secondary antibody was anti-rabbit (@1:5000 dilution). Blots were exposed for various times, depending on strength of signal.

For qPCR for RNA samples, RNA was harvested as described above for qPCR analysis using RNeasy Plus Mini kit (Qiagen). cDNA was synthesized from 400 ng RNA in 20 μl reaction with SuperScript IV VILO Master Mix (Thermo 11756500; diluted 1:4 in water). Native ABCA4 (Thermo commercial assay Hs00979594_m1) spans exons 49-50. For the housekeeper gene as a control, an RNF20 assay was used (Thermo commercial assay Hs00219623_m1). Chimeric ABCA4 codon optimized exon 22—native exon 23——qPCR primers and probes were the following:

Probe (FAM) 063_ABCA4 co22n23_P1:
CGTGGACCCTTACAGCAGAAG
Forward Primer 064_ABCA4 co22n23_F1:
GATCCTGGATGAGCCTAC
Reverse Primer 065_ABCA4 co22n23_R1:
GGACATGATGATGGTTCTG

Duplex with RNF20 assay qPCR primers and probes were the following:

Probe (VIC) 088_RNF20_P2:
CAGCGACTCAACCGACACTT
Forward primer 091_RNF20_F2:
GCAGTGGGATATTGACAA
Reverse primer 099_RNF20_R5:
CGAGCATTGATAGTGATTG

The PCT reaction was run using QuantiFast 2× qPCR Mastermix.

### Results

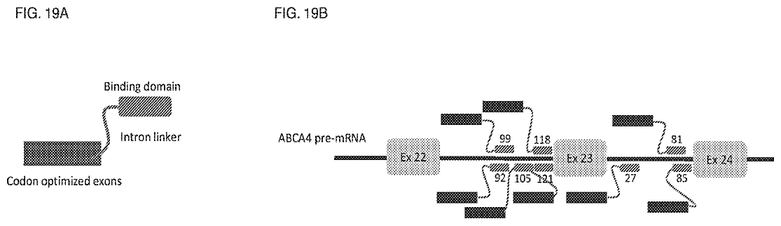

Trans-splicing molecules binding to introns 19, 22, 23, and 24 were tested. No protein restoration was observed in trans-splicing molecules binding to introns 19 and 24 (data not shown), but trans-splicing molecules binding to introns 22 and 23 yielded restoration of ABCA4 protein and RNA expression (FIGS. 19A and 19B), as discussed below.

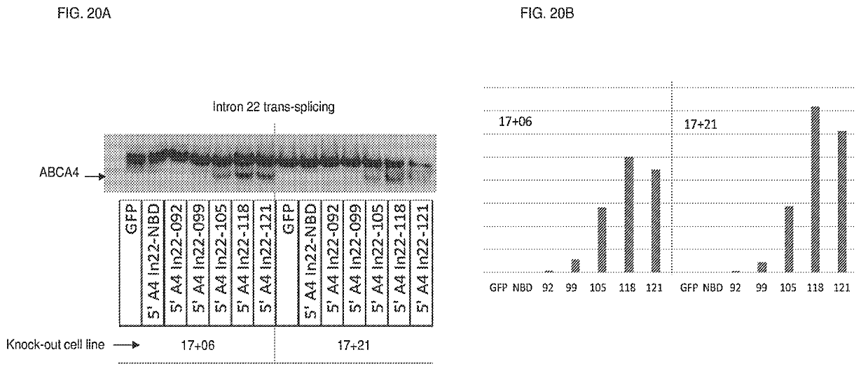

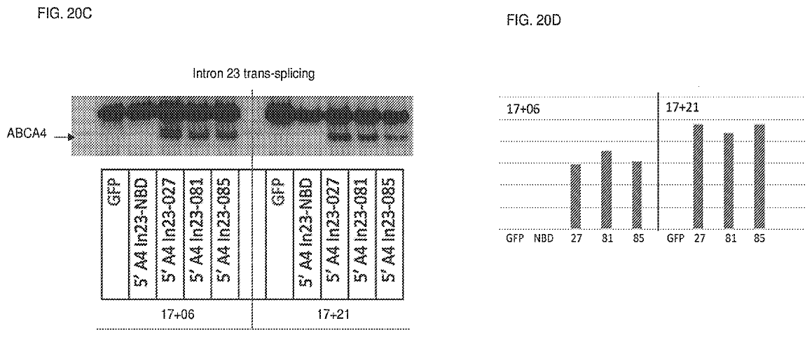

FIG. 20A is a western blot showing ABCA4 protein expression attributed to trans-splicing reactions from two different cell lines (17+06 and 17+21) with a mock GFP control or 5′A4In22 tethered to five different binding domains (No binding domain (NBD) control, 92, 99, 105, 118, and 121, numbering corresponding to FIG. 3 RTM #, where binding domain 92 binds nucleotides 911-1060 of intron 22, binding domain 99 binds nucleotides 981-1130 of intron 22, binding domain 105 binds nucleotides 1041-1190 of intron 22, binding domain 118 binds nucleotides 1171-1320 of intron 22, and binding domain 121 binds nucleotides 1201-1350 of intron 22 (following to the 10-base shift interval of 150-mers across intron 22, described above)). Four of these intron-22 binding constructs, 99, 105, 118, and 121 yielded protein restoration, with 105, 118, and 121 showing particularly enhanced restoration and 118 showing the greatest amount of protein expressed in both cell lines. mRNA expression profiles showed a similar pattern, with the 118 construct yielding the greatest levels of ABCA4 mRNA in both cell lines (FIG. 20B). Units are relative to the RNF20 housekeeping gene.

FIG. 20C is a western blot showing ABCA4 protein expression attributed to trans-splicing reactions from two different cell lines (17+06 and 17+21) with a mock GFP control or 5′A4In23 tethered to three different binding domains (NBD control, 27, 81, and 85, numbering corresponding to FIG. 5 RTM #, where binding domain 27 binds nucleotides 261-410 of intron 23, binding domain 81 binds to 801-950 of intron 23, and binding domain 85 binds 841-990 of intron 23 (following to the 10-base shift interval of 150-mers across intron 23, described above)). All three intron 23-binding constructs yielded trans-splicing as indicated by the amount of protein expressed in both cell lines. mRNA expression profiles yielded similar results, with all three constructs yielding robust ABCA4 mRNA expression in both cell lines (FIG. 20D). Units are relative to the RNF20 housekeeping gene.

Together, the ABCA4 protein and RNA expression data obtained for intron 22- and 23-binding trans-splicing molecules correlate with the binding domain screen described above. In particular, intron 22-binding constructs 105, 118, and 121 and intron 23-binding constructs 27, 81, and 85 were predicted to bind with high efficiency (FIGS. 3 and 5), and the present ABCA4 protein restoration data indicate that the ABCA4 intron regions containing the binding sites of these constructs are amenable to binding to ABCA4 trans-splicing molecules to confer protein and RNA restoration. In the present examples, 10-20% protein expression was restored, with restoration being comparable between intron 22- and 23-binding trans-splicing molecules. Importantly, because ABCA4-related diseases (such as Stargardt Disease) are recessive, asymptomatic carriers of the disease likely express less-than normal ABCA4, and, without wishing to be bound by theory, partial protein restoration, as shown here, is likely to confer a meaningful clinical benefit.

### Example 2. CEP290

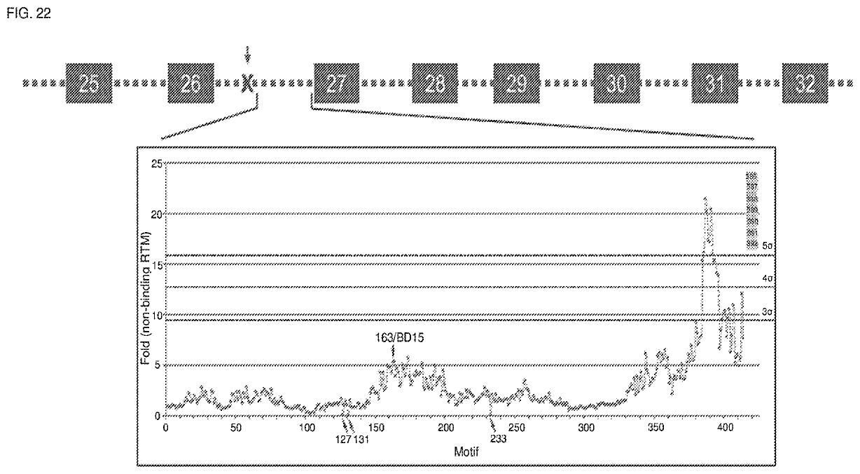

Screening a series of binding domains configured to bind CEP290 intron 26 (SEQ ID NO: 85) at sequential binding sites revealed a region at the 3′ portion of CEP290 intron 26 that was preferentially amenable to trans-splicing of a 5′ trans-splicing molecule—a region from nucleotides 4,980 to 5,838 of intron 26 (FIG. 22). Binding sites within the range of nucleotides from 5,348 to 5,838, from 5,348 to 5,700, from 5,400 to 5,600, from 5,460 to 5,560, or 5,500 were revealed as particularly highly efficient at mediating trans-splicing.

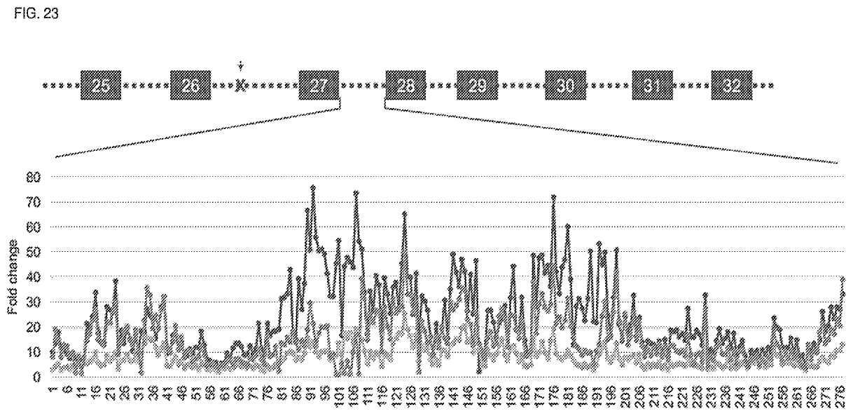

FIG. 23 shows results of a similar screen for CEP290 intron 27 (SEQ ID NO: 86). Binding sites having nucleotides 120 to 680, 710 to 2,200, 2,670 to 2,910 were identified as preferentially suitable for trans-splicing of a 5′ trans-splicing molecule. In particular, binding domains targeted to binding sites within the ranges of nucleotides 790 to 2,100, nucleotides 1,020 to 1,630, or nucleotides 1,670 to 2,000 were highly efficient at trans-splicing.

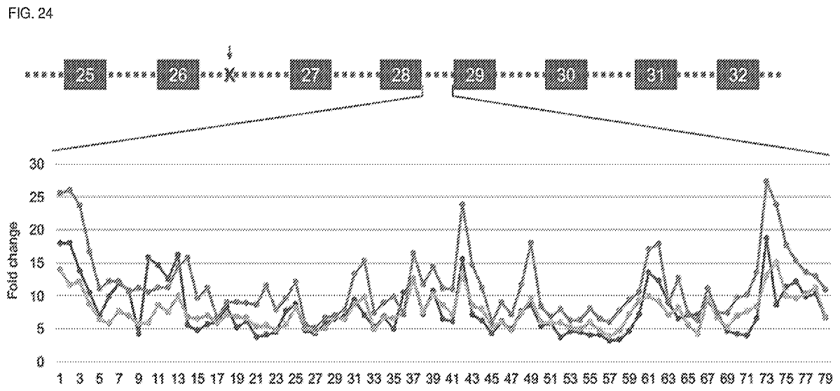

At intron 27 (SEQ ID NO: 87), binding sites within the ranges of nucleotides 1 to 390 (e.g., nucleotides 1 to 200), nucleotides 410 to 560, or nucleotides 720 to 937 were identified as having relatively high efficiency of trans-splicing (FIG. 24).

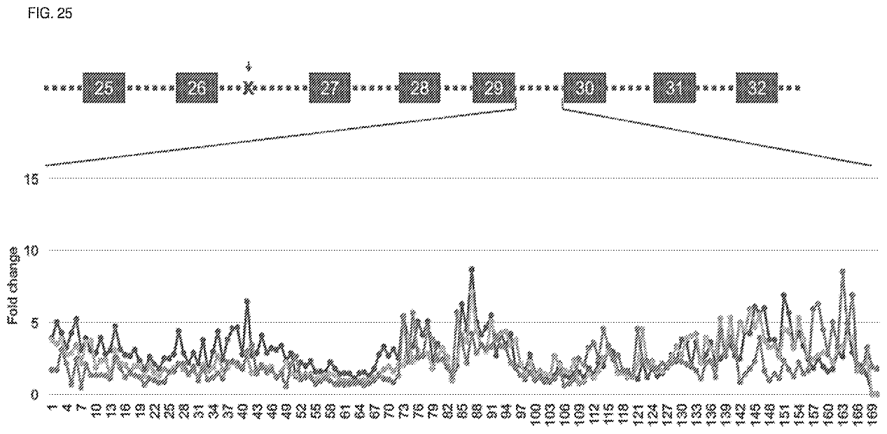

Intron 28 (SEQ ID NO: 88) was similarly characterized and shown to possess relatively efficient binding sites within nucleotides 1 to 600, nucleotides 720 to 940, or nucleotides 1,370 to 1,790 (FIG. 25).

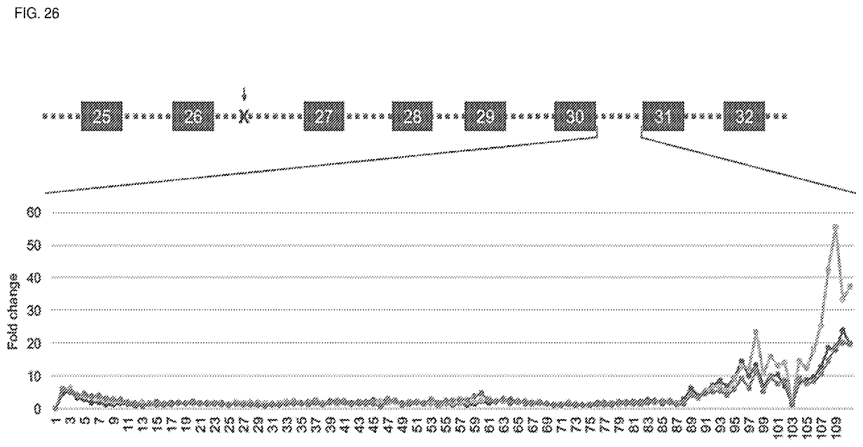

At intron 29 (SEQ ID NO: 89), the 3′ portion of the intron was significantly more efficient at mediating 5′ trans-splicing relative to the remainder of the intron (FIG. 26). In particular, binding domains targeting binding sites within the range of nucleotides 95 to 1,240, e.g., nucleotides 1,060 to 1,240, exhibited the greatest trans-splicing efficiency.

1-72. (canceled)

73. A nucleic acid trans-splicing molecule comprising, operatively linked in a 5′-to-3′ direction:
(a) a binding domain, wherein the binding domain comprises a sequence ranging from 50-300 nucleotides in length and is configured to bind ABCA4 intron 22 at a binding site within nucleotides 60 to 570, 600 to 800, or 900 to 1,350 of SEQ ID NO: 28;
(b) a splicing domain configured to mediate trans-splicing; and
(c) a coding domain comprising functional ABCA4 exons 23-50;
wherein the nucleic acid trans-splicing molecule is configured to trans-splice the coding domain to endogenous ABCA4 exon 22, thereby replacing endogenous ABCA4 exons 23-50 with the functional ABCA4 exons 23-50.

74. The nucleic acid trans-splicing molecule of claim 73, wherein the binding site is within nucleotides 60 to 570 of SEQ ID NO: 28.

75. The nucleic acid trans-splicing molecule of claim 74, wherein the binding site is within nucleotides 70-250 of SEQ ID NO: 28.

76. The nucleic acid trans-splicing molecule of claim 73, wherein the binding site is within nucleotides 900 to 1,350 of SEQ ID NO: 28.

77. The nucleic acid trans-splicing molecule of claim 73, wherein the binding site comprises:
(a) six or more of nucleotides 60 to 570 of SEQ ID NO: 28;
(b) six or more of nucleotides 600 to 800 of SEQ ID NO: 28;
(c) six or more of nucleotides 900 to 1,350 of SEQ ID NO: 28;

78. The nucleic acid trans-splicing molecule of claim 77, wherein the binding domain comprises six or more consecutive nucleic acid residues that are complementary to the six or more nucleotides of the binding site.

79. The nucleic acid trans-splicing molecule of claim 73, wherein the binding domain comprises a sequence ranging from 50-75 nucleotides in length, 75-100 nucleotides in length, 125-150 nucleotides in length, 150-175 nucleotides in length, 175-200 nucleotides in length, 200-250 nucleotides in length, 100-200 nucleotides in length, or 150 nucleotides in length.

80. The nucleic acid trans-splicing molecule of claim 73, wherein the binding domain comprises at least 10, at least 12, at least 15, at least 20, at least 25, at least 30, at least 40, at least 50, at least 60, at least 70, at least 80, at least 90, at least 100, at least 120, at least 150, or at least 200 consecutive nucleotides that are complementary to the binding site.

81. The nucleic acid trans-splicing molecule of claim 73, wherein the binding domain is 80% to 100% complementary to the binding site.

82. The nucleic acid trans-splicing molecule of claim 73, wherein:
(i) the binding domain is 100-200 nucleotides in length;
(ii) the coding domain is a cDNA sequence;
(iii) the coding domain comprises a naturally-occurring sequence;
(iv) the coding domain comprises a codon-optimized sequence;
(v) an artificial intron comprises a spacer sequence;
(vi) the trans-splicing molecule is from 3,000 to 4,000 nucleotides in length;
(vii) the mutation in the ABCA4 gene is associated with Stargardt Disease; and/or
(viii) the mutation is expressed in a photoreceptor cell.

83. A proviral plasmid comprising the nucleic acid trans-splicing molecule of claim 73.

84. An adeno-associated virus (AAV) comprising the nucleic acid trans-splicing molecule of claim 73.

85. The AAV of claim 84, wherein the AAV preferentially targets a photoreceptor cell.

86. The AAV of claim 84, wherein the AAV comprises an AAV5 capsid protein, an AAV8 capsid protein, an AAV8(b) capsid protein, or an AAV9 capsid protein.

87. The AAV of claim 84, comprising an assembled capsid having packaged therein a vector genome comprising an AAV 5′ ITR, the nucleic acid trans-splicing molecule under the operative control of regulatory sequences, and an AAV 3′ ITR.

88. A pharmaceutical composition comprising the nucleic acid trans-splicing molecule of claim 73, or a proviral plasmid thereof, or an AAV plasmid thereof.

89. A method of correcting a mutation in an ABCA4 gene in a target cell of a subject, the method comprising administering to the subject the pharmaceutical composition of claim 88.

90. A method of correcting a mutation in any one or more of ABCA4 exons 23-50 in a subject in need thereof, the method comprising administering to the subject the pharmaceutical composition of claim 88.

91. The method of claim 89, wherein the subject has Stargardt Disease.

92. The method of claim 89, wherein the composition is administered by subretinal injection, intravitreal injection, or intravenous injection.

93. A method of treating a subject having a disorder associated with a mutation in ABCA4, the method comprising administering to the subject the pharmaceutical composition of claim 88.
